(19) United States
(12) Patent Application Publication
(10) Pub. No.: US 2023/0086199 A1
(43) Pub. Date: Mar. 23, 2023

(54) SYSTEMS AND METHODS FOR EVALUATING CAS9-INDEPENDENT OFF-TARGET EDITING OF NUCLEIC ACIDS

(71) Applicants: The Broad Institute, Inc., Cambridge, MA (US); President and Fellows of Harvard College, Cambridge, MA (US)

(72) Inventors: David R. Liu, Cambridge, MA (US); Jordan Leigh Doman, Cambridge, MA (US); Aditya Raguram, Cambridge, MA (US)

(73) Assignees: The Broad Institute, Inc., Cambridge, MA (US); President and Fellows of Harvard College, Cambridge, MA (US)

(21) Appl. No.: 17/779,953

(22) PCT Filed: Nov. 25, 2020
(86) PCT No.: PCT/US2020/062428

§ 371 Date: May 25, 2022

Related U.S. Application Data
(60) Provisional application No. 62/940,859, filed on Nov. 26, 2019.

(51) Int. Cl. C12Q 1/6827; C12N 9/22; C12N 15/11

(52) U.S. Cl. CPC C12Q 1/6827; C12N 9/22; C12Y 305/04005; C12N 15/111; C12N 2310/20

(57) ABSTRACT

The instant specification provides novel assays and systems for determining off-target effects of base editors. These assays and systems may comprise bacterial and/or eukaryotic cell systems and may be used to determine off-target editing frequencies, including Cas9-independent off-target editing frequencies. Also provided herein are novel base editors, wherein the base editors have reduced Cas9-independent off-target editing frequencies while maintaining high on-target editing efficiencies. Further provided are methods of contacting a nucleic acid molecule with these base editors to obtain reduced off-target editing frequencies, and in particular reduced Cas9-independent off-target editing events. Further provided are methods of treatment comprising administering these base editors to a subject. Also provided are pharmaceutical compositions comprising the base editors described herein, and nucleic acids, vectors, cells, and kits useful for the generation of these base editors.

### RELATED APPLICATIONS

This application claims priority under 35 U.S.C. § 119(e) to U.S. Provisional Application, U.S. Ser. No. 62/940,859, filed Nov. 26, 2019, which is incorporated herein by reference.

### FEDERALLY SPONSORED RESEARCH

This invention was made with government support under grant numbers AI142756, HG009490, and GM118062 awarded by the National Institutes of Health. The government has certain rights in the invention.

### BACKGROUND OF THE INVENTION

Targeted editing of nucleic acid sequences, including the targeted cleavage or targeted introduction of a specific modification into genomic DNA, is a highly promising approach for the study of gene function and also has the potential to provide new therapies for genetic diseases, including those caused by point mutations. Point mutations represent the majority of known human genetic variants associated with disease. Developing robust methods to introduce and correct point mutations is therefore important in understanding and treating diseases with a genetic component. Base editors can perform remarkably clean and efficient nucleobase conversions in target DNA sequences with very low levels of undesirable by-products. However, unintended editing of off-target bases does occur at low frequencies.

Off-target base editing can arise from Cas9/guide RNA-dependent or Cas9-independent editing events. The former result from RNA-guided binding of the Cas9 domain to DNA sites that are similar, but not identical, to the target DNA locus. The latter arise from stochastic associations of base editors with DNA sites that do not have a high degree of sequence identity to the target locus due to an intrinsic affinity of the base editor, particularly when overexpressed, for DNA.

There is an unrecognized need in the art for assays and systems to evaluate the frequency of off-target base editing, as well as base editors that have reduced Cas9-independent off-target editing frequency while retaining high on-target editing efficiency.

### SUMMARY OF THE INVENTION

Various base editors (BEs) have been recently developed. Reference is made to Komor, A. C. et al., Improved base excision repair inhibition and bacteriophage Mu Gam protein yields C:G-to-T:A base editors with higher efficiency and product purity, Sci. Adv. 3 (2017); Rees, H. A. et al., Improving the DNA specificity and applicability of base editing through protein engineering and protein delivery, Nat. Commun. 8, 15790 (2017); U.S. Patent Publication No. 2018-0073012, published Mar. 15, 2018; U.S. Patent Publication No. 2017-0121693, published May 4, 2017; International Publication No. WO 2017/070633, published Apr. 27, 2017; U.S. Patent Publication No. 2015-0166980, published Jun. 18, 2015; U.S. Pat. No. 9,840,699, issued Dec. 12, 2017; and U.S. Pat. No. 10,077,453, issued Sep. 18, 2018; International Patent Publication No. WO 2020/102659, published May 22, 2020; International Patent Publication No. WO 2020/181180, published Sep. 10, 2020; and International Patent Publication No. WO 2020/214842, published Oct. 22, 2020; each of which is incorporated herein by reference. Base editors are fusions of a Cas (“CRISPR-associated”) domain and a nucleotide modification domain (e.g., a natural or evolved deaminase, such as a cytidine deaminase, e.g., APOBEC1 (“apolipoprotein B mRNA editing enzyme, catalytic polypeptide 1”), CDA (“cytosine deaminase”), and AID (“activation-induced cytidine deaminase”)) domains. In some cases, base editors may also include proteins or domains that alter cellular DNA repair processes to increase the efficiency and/or stability of the resulting single-nucleotide change.

Two classes of base editors have been generally described to date: cytosine base editors, which convert target C:G base pairs to T:A base pairs, and adenosine base editors, which convert A:T base pairs to G:C base pairs. Collectively, these two classes of base editors enable the targeted installation of all possible transition mutations (C-to-T, G-to-A, A-to-G, T-to-C, C-to-U, and A-to-U), which collectively account for about 61% of known human pathogenic single nucleotide polymorphisms (SNPs) in the ClinVar database. See Gaudelli, N. M. et al., Programmable base editing of A:T to G:C in genomic DNA without DNA cleavage. Nature 551, 464-471 (2017), which is incorporated herein by reference. In particular, C-to-T base editors use a cytidine deaminase domain to convert cytidine to uridine in the single-stranded DNA loop created by the Cas9 (“CRISPR-associated protein 9”) domain. The opposite strand is nicked by Cas9 to stimulate DNA repair mechanisms that use the edited strand as a template, while a fused uracil glycosylase inhibitor slows excision of the edited base. Eventually, DNA repair leads to a C:G to T:A base pair conversion. This class of base editor is described in U.S. Patent Publication No. 2017/0121693, published May 4, 2017, which issued on Jan. 1, 2019, as U.S. Pat. No. 10,167,457, each of which is incorporated herein by reference.

Cytosine base editors (CBEs), fusions of catalytically impaired Cas9 proteins, cytidine deaminases, and uracil glycosylase inhibitors, enable the targeted conversion of C:G to T:A base pairs in genomic DNA. Recent studies report that BE3, the original CBE, can induce a low frequency of off-target Cas9-independent DNA deamination in mouse embryos and in rice. See Y. Zong, Y. et al. Nat. Biotechnol. 35, 438-440 (2017).

Accordingly, the present disclosure describes multiple assays that measure the propensity of different base editors to induce Cas9-independent deamination in E. coli and in human cells, such as methods that do not require whole-genome sequencing, are described herein. These methods enable the identification of base editors that exhibit reduced levels of Cas9-independent deamination, and also display restricted on-target activity either in the form of a narrowed on-target editing window or lower average editing efficiency.

The present disclosure further describes novel CBE variants that exhibit increased on-target editing efficiency while maintaining minimized off-target DNA editing relative to existing CBEs. These novel CBE variants comprise novel combinations of mutant cytidine deaminases, such as the YE1, YE2, YEE, and R33A deaminases, and Cas9 domains, and/or novel combinations of mutant cytidine deaminases, Cas9 domains, uracil glycosylase inhibitor (UGI) domains and nuclear localizations sequence (NLS) domains. The suite of CBEs characterized and engineered herein collectively offer ˜10- to 100-fold lower average Cas9-independent off-target DNA editing, as well as ˜5- to 50-fold average lower Cas9-dependent off-target DNA editing, while maintaining robust on-target editing at most positions targetable by canonical CBEs. The novel CBEs of the present disclosure are especially promising for base editing applications in which off-target editing must be minimized, such as editing of genes that are highly expressed and genese associated with diseases or disorders, such as cancer.

The disclosed CBEs do not suffer from higher levels of other forms of undesired editing, in addition to exhibiting lower Cas9-independent off-target editing. The disclosed CBEs exhibit fewer insertions and/or deletions (indels), less Cas9-dependent DNA off-target editing, and less RNA off-target editing, following their use in methods of editing target sequences in nucleic acids.

The disclosed CBEs comprise fusion proteins comprising a cytidine deaminase fused to a catalytically impaired Cas9 protein and one or more copies of a UGI (1, 2). Deamination of cytosine within a base editing activity window (canonically, protospacer positions ˜4-8, counting the PAM as positions 21-23) in the single-stranded DNA loop displaced by the Cas9 guide RNA generates uracil, which is partially protected from base excision by the UGI. Selective nicking of the opposite DNA strand biases cellular DNA repair to replace the non-edited strand, resulting in the conversion of a target C:G base pair to a T:A base pair (1, 3, 4). CBEs have achieved high levels of nucleobase conversion with low levels of indels in numerous cell types and organisms, including animal models of human genetic diseases (4-8).

Like other Cas9-directed genome editing tools, base editors can bind to off-target genomic loci that have relatively high sequence homology (greater than about 60% sequence identity) to the target protospacer. A subset of these Cas9-dependent off-target binding events can lead to base editing (1, 9-11). Although typically a subset of off-target DNA modification by the corresponding Cas9 domain (1, 10, 12), Cas9-dependent off-target base editing can be minimized by using Cas9 variants with higher DNA specificity, and/or by delivering base editors as transient protein: RNA complexes, rather than expressing them from longer-lived DNA constructs (12).

The present disclosure is based, at least in part, on the finding that, in addition to Cas9-dependent off-target base editing, deamination from Cas9-independent binding of a base editor's deaminase domain to DNA represents a distinct type of off-target base editing.

Unlike Cas9-dependent off-target editing, Cas9-independent deamination occurs at different loci between samples, making it difficult to characterize by targeted high-throughput sequencing. Extensive whole-genome sequencing experiments such as those performed by Zuo et al. and Jin et al. are low-throughput and expensive, limiting their use for evaluating and engineering base editors with decreased Cas9-independent deamination activity. The present disclosure provides methods to efficiently evaluate the propensity of a base editor to cause Cas9-independent editing (i.e., Cas9-independent deamination) is described, as is the application of these methods to identify and engineer base editor variants that minimize Cas9-independent DNA editing. The described methods are suitable for use in prokaryotic cells such as E. coli, or eukaryotic cells such as mammalian cells.

Accordingly, the present disclosure provides assays for measuring the Cas9-independent off-target editing frequencies of base editors. Thus, provided herein are methods for determining the off-target effects of a base editor, wherein the methods comprise (a) contacting a nucleic acid molecule comprising a target sequence with a first complex, wherein the first complex comprises (i) a base editor comprising a Cas9 domain, and (ii) a first guide RNA that is engineered to bind to the Cas9 domain of the cytosine base editor, wherein the first guide RNA is complementary to the target sequence; (b) contacting the nucleic acid molecule with a second complex, wherein the second complex comprises (iii) a first nuclease inactive Cas9 (dCas9) protein, and (iv) a second guide RNA that is engineered to bind to the first dCas9 protein, wherein the second guide RNA is complementary to an off-target sequence, whereby the first complex and second complex create two or more R-loops in the nucleic acid molecule. The methods further include a step of (c) sequencing at least a portion of the target sequence and/or at least a portion of the nucleic acid molecule comprising the off-target sequence. The off-target sequence may comprise about 60% or less sequence identity to the target sequence. The methods may further comprise contacting the nucleic acid molecule with additional complexes (e.g., up to six complexes) that comprise a second dCas9 protein and a third guide RNA that is engineered to bind to the second dCas9 protein, wherein the third guide RNA is also complementary to the off-target sequence.

The step of contacting in the described method may include a step of transfecting the cell with one or more nucleic acid vectors (e.g. plasmids) encoding the base editor, the first guide RNA, the first dCas9 protein, the second guide RNA, the second dCas9 protein, and/or the third guide RNA. One or more of these molecules may be encoded on the same vector. The methods may be performed using lipofection, nucleofection, or electroporation, in a population of cells, such as mammalian cells.

In other aspects, the disclosure provides systems for determining off-target editing effects of a base editor that comprise one or more cells having i) a first nucleic acid molecule encoding a base editor comprising a Cas9 domain; (ii) a second nucleic acid molecule encoding a first guide RNA that is engineered to bind to the Cas9 domain of the base editor and is complementary to a target sequence; (iii) a third nucleic acid molecule encoding a dCas9 protein; and (iv) a fourth nucleic acid molecule encoding a second gRNA that is engineered to bind to the dCas9 protein, wherein the second guide RNA that is complementary to an off-target sequence. The off-target sequence may comprise about 60% or less sequence identity to the target sequence. The Cas9 domain may be derived from a first bacterial species (e.g., S. pyogenes Cas9, or SpCas9), and the dCas9 protein may be derived from a second bacterial species (e.g., S. aureus Cas9, or SaCas9). The Cas9 domain may comprise a Cas9 nickase. These systems and methods may be adapted for use in mammalian cells, such as human cells.

In another aspect, the present disclosure provides bacterial systems for determining off-target editing that comprise one or more prokaryotic (e.g., bacterial) cells comprising (i) a first nucleic acid molecule that contains a target sequence within a first inactive antibiotic resistance gene, wherein the target sequence within the first inactive antibiotic resistance gene contains a first mutant nucleotide base that yields an active antibiotic resistance gene conferring resistance to a first antibiotic when the first mutant nucleotide base is mutated to a different nucleotide base; (ii) a second nucleic acid molecule that contains a non-target sequence within a second inactive antibiotic resistance gene, wherein the non-target sequence within the second inactive antibiotic resistance gene contains a second mutant nucleotide base that yields an active antibiotic resistance gene conferring resistance to a second antibiotic when the second mutant nucleotide base is mutated to a different base; and (iii) a third nucleic acid molecule encoding a base editor and a guide RNA that is complementary to the target sequence within the first inactive antibiotic resistance gene. The present disclosure also provides assay methods in accordance with the described systems comprising contacting a prokaryotic cell that comprises the second nucleic acid molecule with (i) the first nucleic acid molecule, and (ii) the third nucleic acid molecule; and further contacting the prokaryotic cell with a growth medium comprising the second antibiotic and/or the first antibiotic.

In other aspects, the disclosure provides novel CBEs that exhibit reduced off-target base editing frequencies (such as Cas9-independent off-target editing) while maintaining high on-target editing efficiencies. The described base editors may comprise a cytidine deaminase selected from YE1, YE2, YEE, EE, R33A, R33A+K34A, AALN, APOBEC3A (A3A and eA3A), or APOBEC3G (A3G), and variants thereof, as well as one or more nuclear localization signals and two or more uracil glycosylase inhibitor (UGI) domains. In some embodiments, the disclosed cytosine base editors comprise evolved nucleic acid programmable DNA binding proteins (napDNAbp), such as an evolved Cas9. In some embodiments, the disclosed CBEs recognize an expanded PAM and/or make edits in a narrower target window. In certain embodiments, the cytidine deaminase domain is selected from R33A, YE1, YE2, YEE, or EE, or a variant thereof, and the napDNAbp domain is selected from an nCas9, an xCas9, an SpCas9-NG, or a CP1028. In some embodiments, the napDNAbp domain is selected from any one of the amino acid sequences set forth in SEQ ID NOs: 213-229 or 235-237. Exemplary base editors may comprise any one of the amino acid sequences set forth in SEQ ID NOs: 257-282.

In other aspects, the disclosure provides methods of editing a target nucleobase pair in a nucleic acid molecule (or substrate) that result in low off-target editing frequencies, such as low Cas9-independent off-target editing frequencies. Cytosine base editors with Cas9-dependent off-target editing frequencies of about 2.0% to about 15% were recently described in Huang, T. P., et al., Nat. Biotechnol. 37, 626-631 (2019), incorporated herein by reference. And CBEs with apparent on-target editing efficiencies in vivo of about 50% have been described in International Application No PCT/US2019/033848, published as WO/2019/226953 on Nov. 28, 2019, and Komor et al., Sci. Adv. 2017; 3:eaao4774, each of which is incorporated herein by reference. The methods described herein comprise contacting a target sequence in a nucleic acid molecule with any one of the base editors described herein associated with a guide RNA (gRNA), obtaining a successful edit (e.g., deaminating a cytosine in the case of cytosine base editors, or deaminating an adenine in the case of adenine base editors) in a target nucleobase pair within the target sequence, and obtaining a frequency (such as an average frequency) of off-target editing of less than 1.5% (such as less than 1.25%, less than 1.0%, less than 0.75%, or less than 0.5%). These methods may further comprise obtaining an on-target editing efficiency (i.e., a frequency of intended deamination of a cytosine in the target nucleobase pair) of greater than 50% (such as greater than 60%, greater than 70%, or greater than 85%) at the target nucleobase pair. These methods may further comprise obtaining an on-target editing efficiency of greater than 50% and a frequency of off-target editing of less than 1.5%. These methods may further comprise obtaining an on-target editing efficiency of greater than 60% and a frequency of off-target editing of less than 0.75%. The editing efficiencies and off-target editing frequencies described herein may be determined by performing high-throughput sequencing of the nucleic acid substrates at the appropriate target site(s) and known off-target site(s) following the step of contacting the nucleic acid molecule with the base editor of interest. This step of performing high-throughput sequencing may include a whole genome sequencing (WGS) step.

The step of contacting a nucleic acid molecule of the disclosed methods may be performed in vivo, such as at a target sequence in the genome of a subject, such as a human. In some embodiments, the subject is a non-human animal, such as a non-human mammal. The step of contacting may be performed in vitro, such as in a cell. In some embodiments, the step of contacting is performed in a mammalian cell, such as a non-human cell. In some embodiments, the step of contacting is performed in cells derived from a non-human animal.

In other aspects, the disclosure provides polynucleotides and vectors comprising a polynucleotide encoding any of the novel base editors described herein. The disclosure also provides complexes containing any one of the CBEs described herein in association with a gRNA. The disclosure further provides cells (such as mammalian cells) comprising any of the CBEs described herein, polynucleotides, vectors, or complexes.

In other aspects, the disclosure provides pharmaceutical compositions comprising the described base editors. The disclosed pharmaceutical compositions may comprise any of the described base editors and a pharmaceutically acceptable excipient, and optionally a guide RNA (gRNA).

The present disclosure further provides kits for use of the base editors described herein in targeted nucleic acid editing, as well as kits for use of evaluating the off-target effects of these base editors. The disclosed kits for use of the described base editors in nucleic acid editing may comprise a nucleic acid construct comprising nucleotide sequences encoding any one of the disclosed base editors and one or more gRNAs having complementarity to a target sequence.

Further disclosed herein are kits for performing the methods of evaluating off-target effects, as described herein. Exemplary such kits comprise nucleic acid constructs including (i) a nucleic acid sequence encoding a cytosine base editor as described herein; (ii) a nucleic acid sequence encoding a first gRNA that is engineered to bind to the Cas9 domain of the cytosine base editor and is complementary to a target sequence of interest; (iii) a nucleic acid sequence encoding a first dCas9 protein; and (iv) a nucleic acid sequence encoding a second gRNA that is engineered to bind to the dCas9 protein and is complementary to an off-target sequence.

In some aspects, the base editors described herein may be administered to a subject to treat a disease or disorder. Thus, methods are provided wherein the described CBEs are administered to a subject, and a target sequence in the genome of the subject is edited with high on-target frequency and reduced Cas9-independent off-target frequency. The target sequence may comprise a mutant C:G base pair, e.g., a mutant C:G base pair associated with a disease or disorder.

The disclosure further provides uses of any one of the base editors described herein and a guide RNA targeting this base editor to a target C:G base pair in a nucleic acid molecule in the manufacture of a kit for nucleic acid editing, wherein the nucleic acid editing comprises contacting the nucleic acid molecule with the base editor and guide RNA under conditions suitable for the deamination of the cytosine (C) of the C:G nucleobase pair. The disclosure further provides uses of any one of the base editors described herein and a guide RNA targeting this base editor to a target C:G base pair in a nucleic acid molecule in the manufacture of a kit for evaluating the off-target effects of the base editor.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

FIGS. 1A-1C show on-target and Cas9-independent off-target DNA editing in E. coli. FIG. 1A is a schematic of the experimental design for the prokaryotic cell assays of the disclosure. FIG. 1B is a graph showing assay validation for on-target and off-target (indicated with the “#” symbol) DNA editing. The fraction of resistant colonies was calculated relative to the number of E. coli plated on maintenance antibiotics. Data are shown as individual data points and mean±SEM for n=3 or n=15 bacterial colonies. E63A refers to a catalytically inactivated APOBEC1 E63A mutant. FIG. 1C is a graph showing the performance of CBEs that use alternative deaminases. All constructs are of the deaminase-dCas9-UGI (BE2) architecture. The dotted line indicates the background level of rifampin resistance of the inactive APOBEC1 E63A deaminase control. Data are shown as individual data points and mean±SEM for n=3 or n=15 bacterial colonies.

FIGS. 2A-2E show Cas9-independent deamination by cytosine base editors (CBEs) in HEK293T cells. FIG. 2A is a schematic showing the BE4max-like architecture for CBE constructs used in mammalian cell experiments. FIG. 2B is a schematic of the experimental design for the eukaryotic cell assays of the disclosure. The cytidine deaminase domain of a CBE containing S. pyogenes Cas9 (SpCas9) nickase deaminate cytosines within R-loops generated by isolated dead S. aureus Cas9 (dSaCas9) in a Cas9-independent manner. R-loops are generated by isolated dSaCas9 at known off-target sites by virtue of the hybridization to these sites of “off-target” guide RNAs that are engineered to bind SaCas9 specifically. Meanwhile, the SpCas9 nickase domain of the CBE generates an R-loop at the target site by virtue of the hybridization to the target site of an on-target guide RNA that is engineered to bind SpCas9 specifically. The two uracil glycosyase inhibitor (UGI) domains of exemplary CBEs are also shown. FIG. 2C and FIG. 2D are collections of graphs showing Cas9-independent off-target C:G-to-T:A editing frequencies detected by targeted high-throughput sequencing of six dSaCas9 loci following co-transfection with SpCas9-targeted CBEs. Each subplot shows the observed C:G-to-T:A conversion of a single underlined cytosine and its immediate sequence context. Transfections in FIG. 2C were performed with one of two SpCas9 sgRNAs (targeting the RNF2 or EMX1 genomic loci) or no SpCas9 sgRNA, with on-target editing controls shown in FIG. 7A. Transfections in FIG. 2D were performed with one SpCas9 sgRNA targeting the RNF2 genomic locus, with on-target editing controls shown in FIG. 7B. For FIG. 2C and FIG. 2D, data are shown as individual data points and means±SEM for n=3 independent biological replicates performed on different days. FIG. 2E is a schematic showing the mechanism by which “off-target” guide RNAs may bind SaCas9 specifically, while on-target guide RNAs may bind SpCas9 specifically. Guide RNAs may be engineered so that their backbone (or “core”) sequences (the stem-loop portions of the RNA molecules shown) can be modified to interact with a binding pocket present in an SpCas9 protein or SaCas9 protein. The gRNA that is specific for the SpCas9 protein (light blue) does not interact with the binding pocket of the SaCas9 protein (red), and vice versa.

FIGS. 3A-3D show that YE1 balances efficient on-target editing with greatly decreased Cas9-independent editing as confirmed by whole-genome sequencing (WGS). FIG. 3A shows on-target editing versus average off-target editing for all CBEs in this study. The y-axis reflects the mean on-target control editing at the on-target RNF2 locus used in the orthogonal R-loop assay, and the x-axis reflects the mean off-target editing for six orthogonal R loops. The box indicates CBE variants that have substantially decreased Cas9-independent off-target editing but retain appreciable on-target activity. See FIGS. 8A-8D and FIG. 2D for mean values and SEM at individual sites. FIG. 3B shows the average maximum on-target and average off-target editing for constructs with decreased Cas9-independent editing events. The y-axis reflects average editing across six on-target protospacers of the most highly edited cytosine within that protospacer. The x-axis reflects the average off-target editing in the orthogonal R-loop assay. See FIG. 13 and FIG. 2D for mean values and SEM at individual sites. FIG. 3C shows the number of C•G-to-T•A single-nucleotide variants (SNVs) relative to the initial parent sample detected by whole-genome sequencing (WGS). The fraction of cytosine base SNVs that can be targeted for editing with either of the indicated CBEs are indicated with the “#” and “*” symbols. FIG. 3D shows the total number of SNVs relative to the initial parent sample detected by WGS. Each dot (•) represents the number of SNPs called in a clonal population of cells relative to the parent sample. Each clonal population was derived from a single GFP-positive cell that was isolated after flow cytometry sorting of HEK293T cells transfected with a CBE-P2A-GFP construct for the GFP-positive cells. Horizontal lines and error bars indicate mean number of SNVs±SEM for n=8 (BE4 and YE1) or n=7 (Cas9 nickase). P values were calculated using the Mann-Whitney U test. **p<0.005; ***p<0.0005.

FIG. 4 shows the Sanger sequencing of the rpoB gene from rifampin-resistant E. coli colonies. Sanger sequencing traces from three unique rifampin-resistant colonies that showed different C:G to T:A mutations. Top to bottom, left to right the sequences correspond to SEQ ID NOs: 1-15.

FIGS. 5A-5B show an analysis of point mutations (single nucleotide variations, or SNVs) reported by Zuo et al. (13). The sequence context of C•G-to-T•A SNVs identified by Zuo et al. in BE3-treated mouse embryos, as shown in FIG. 5A, or Cas9-, Cre-, and ABE-treated mouse embryos, as shown in FIG. 5B, is shown as sequence logos. Using the genomic locations of all C:G-to-T:A SNVs reported by Zuo et al., the flanking sequences (20 base pairs on either side) were extracted from the mouse mm10 reference genome [GCA_000001635.2].

FIG. 6 shows the relationship between on-target editing and off-target editing for rifampin resistance assay in bacteria. Mean values are plotted for both on-target and off-target editing and were calculated as the number of resistant bacteria relative to the number of bacteria plated on maintenance antibiotics. Replicates used for this calculation are shown in FIG. 1C.

FIGS. 7A-7B show the on-target DNA editing controls in HEK293T cells. On-target DNA base editing efficiencies at the appropriate genomic loci corresponding to the SpCas9 sgRNAs included in the dSaCas9+ SpCas9-targeted CBE co-transfection experiments in FIG. 2C's RNF2 and EMX1 sites, as shown in FIG. 7A, and FIG. 2D's RNF2 site, as shown in FIG. 7B. The on-target editing efficiency for each SpCas9-targeted CBE when combined with each of the six R-loop-generating SaCas9 sgRNAs was determined by high-throughput sequencing to ensure that any absence of SpCas9-independent off-target editing was not due to lack of CBE expression or poor transfection efficiency. Data are shown for a single cytosine (C6) in the editing window as individual data points and mean values±SEM for n=3 biological replicates performed at different times.

FIGS. 8A-8D show the in vitro characterization of CBEs. FIG. 8A shows the denaturing PAGE gel of purified CBEs. Purified proteins from left to right are APOBEC1-dCas9-UGI (APO1), YE1-dCas9-UGI (YE1), and APOBEC3A-dCas9-UGI (A3A). FIG. 8B shows the representative denaturing PAGE gel of in vitro deamination reaction products following incubation with base editors (CBEs) at the denoted concentrations and treatment with USER enzyme. The reactions shown were performed with 25 μM ssDNA substrate for 30 minutes. ‘C’ refers to the substrate-only control (with no CBE added), and ‘U’ refers to the positive control with a uracil synthetically incorporated in place of the single cytosine in the ssDNA oligonucleotide. FIG. 8C shows a Michaelis-Menten kinetic analysis of in vitro deamination reaction rates determined by calculating the ratio of the intensities of the cleaved product and substrate bands by gel densitometry. Data are shown as mean values±SD for three technical replicates. FIG. 8D shows the calculated values for kinetic parameters from non-linear regression using the Michaelis-Menten equation.

FIGS. 9A-9C show the intracellular deamination of a co-transfected ssDNA oligonucleotide by CBE variants in HEK293T cells. FIG. 9A is a graph showing the observed deamination levels detected by high-throughput sequencing at all 5′ TC cytosines within a co-transfected ssDNA oligonucleotide in HEK293T cells. FIG. 9B is a scatter plot of mean deamination levels for the twelve cytosines shown in FIG. 9A. FIG. 9C is a graph showing on-target genomic DNA C:G-to-T:A editing controls at C6 of the HEK2 locus. Data are shown as individual data points and mean values±SEM for n=3 biological replicates performed at different times.

FIGS. 10A-10B show the Cas9-independent off-target editing and on-target editing as well as the cytosine base editors targeted with SpCas9-NG in HEK293T cells. FIG. 10A is a collection of graphs showing off-target editing of BE4-like constructs modified with SpCas9-NG at R-loops generated by dSaCas9 and one of six SaCas9 sgRNAs. FIG. 10B is a collection of graphs showing the on-target editing controls at C6 of the RNF2 locus. Data are shown as individual data points and mean values±SEM for n=3 biological replicates performed at different times by different researchers.

FIG. 11 shows on-target base editing by circularly permuted CBE variants in HEK293T cells. This data is summarized in the heat map of FIG. 3B. Data are shown as individual data points and mean values±SEM for n=3 biological replicates performed at different times.

FIGS. 12A-12D show the base editing activity windows for BE4max, YE1max, and YE1-CP1028 in HEK293T cells. FIG. 12A shows the mean editing at various cytosines across six sites tested were grouped by the position of the cytosine within the protospacer (counting the PAM as positions 21-23) and averaged. FIG. 12B shows the same data as in FIG. 12A, but is normalized to peak editing at a given site. For each edited cytosine, the editing efficiency was divided by the editing efficiency of the most highly edited cytosine within that protospacer. FIG. 12C is a table showing the number of cytosines analyzed for each window position. FIG. 12D shows the on-target editing at a single protospacer that contains a multi-C repeat. “YE1max” refers to a BE4max architecture comprising a YE1 deaminase domain.

FIG. 13 shows on-target editing by R33A+K34A-BE4 and AALN-BE4 in HEK293T cells. (“AALN” refers to the R33A+K34A+H122L+D124N variant of rAPOBEC1.) Data are shown as individual data points and mean values±SEM for n=3 biological replicates performed at different times.

FIGS. 14A-14C are graphs showing that AALN shows minimal Cas9-independent off-target editing in E. coli or mammalian cells. FIG. 14A shows the Rifampin assay for AALN-BE2 compared to a catalytically inactivated APOBEC1 E63A mutant base editor. Data are shown as individual data points and mean values±SEM for n=3 bacterial colonies. Bars representing colonies in which off-target editing occurred are indicated with the * symbol. FIG. 14B shows the on-target editing efficiency for the RNF2 locus in HEK293T cells using various SaCas9 sgRNAs to create an orthogonal R-loop. Data are shown as individual data points and mean values±SEM for n=3 biological replicates performed at different times. FIG. 14C shows off-target data editing of AALN-BE4 in HEK293T cells compared to a no-editor control and to R33A+K34-BE4. On-target editing data for R33A+K34A-BE4 and for the no-editor control are in FIGS. 7A-7B. Data are shown as individual data points and mean values±SEM for n=3 biological replicates performed at different times.

FIGS. 15A-15B show that circularly-permuted CBE variants show minimal Cas9-independent off-target editing in HEK293T cells. FIG. 15A is a collection of graphs showing SpCas9-independent off-target editing of BE4-CP1028 constructs (which use the circularly permuted SpCas9-CP1028 for targeting) at six R-loops created by dSaCas9 and one or more of the sgRNAs engineered to bind S. aureus. FIG. 15B shows the on-target editing controls at C6 of the RNF2 locus. Data are shown as individual data points and mean values±SEM for n=3 biological replicates performed at different times by different researchers.

FIG. 16 is a collection of graphs which show CBEs with minimal Cas9-independent off-target editing show low levels of indel formation in HEK293T cells. Data are shown as individual data points and mean values±SEM for n=3 biological replicates performed on different days by different researchers.

FIG. 17 is a collection of graphs which show CBEs with minimal Cas9-independent off-target DNA editing show reduced levels of Cas9-dependent off-target DNA editing in HEK293T cells. The 20 sites shown are the 20 most highly edited off-target substrates of SpCas9 and the EMX1, HEK3, or HEK4 sgRNAs by GUIDE-seq (42). Individual data points and mean values±SEM for n=3 biological replicates performed on different days by different researchers are shown.

FIGS. 18A-18D are graphs showing the protein delivery of the base editor reduces off-target editing while maintaining robust on-target editing in HEK293T cells. FIG. 18A shows off-target editing of BE4 delivered into HEK293T cells as either plasmid or protein, with an on-target SpCas9 sgRNA targeting the RNF2 locus. Individual data points and mean values±SEM for n=3 biological replicates performed at different times are shown. FIG. 18B shows off-target editing of BE4 delivered into HEK293T cells as either plasmid or protein, with an on-target SpCas9 sgRNA targeting the EMX1 locus. Data are shown as individual data points and mean values±SEM for n=3 biological replicates performed at different times. FIG. 18C shows on-target editing controls for the HEK3 off-target analysis. Data is shown as mean±SEM for n=3 biological replicates performed at different times. FIG. 18D shows on-target editing controls for the FANCF off-target analysis. Data is shown as mean±SEM for n=3 biological replicates performed at different times.

FIGS. 19A-19D show that YE1 balances efficient on-target editing with greatly decreased Cas9-independent editing as confirmed by whole genome sequencing (WGS). FIG. 19A is a graph showing on-target editing versus average off-target editing for all CBEs. The y-axis reflects the mean on-target control editing at the on-target RNF2 locus used in the orthogonal R-loop assay, and the x-axis reflects the mean off-target editing for six orthogonal R loops. The box indicates CBE variants that have substantially decreased Cas9-independent off-target editing but retain appreciable on-target activity. See FIGS. 8A-8D and FIG. 2D for mean values and SEM at individual sites. FIG. 19B shows the average maximum on-target and average off-target editing for constructs with decreased Cas9-independent editing events. The y-axis reflects average editing across six on-target protospacers of the most highly edited cytosine within that protospacer. The x-axis reflects the average off-target editing in the orthogonal R-loop assay. See FIG. 13 and FIG. 2D for mean values and SEM at individual sites. FIG. 19C shows the number of C•G-to-T•A single-nucleotide variants (SNVs) relative to the initial parent sample detected by WGS. FIG. 19D shows the total number of SNVs relative to the initial parent sample detected by WGS. For both FIGS. 19C and 19D, each dot represents the number of SNPs called in a clonal population of cells relative to the parent sample. Each clonal population was derived from a single GFP-positive cell that was isolated after flow sorting HEK293T cells transfected with a CBE-P2A-GFP construct for the GFP-positive cells. Horizontal lines and error bars indicate mean number of SNVs±SEM for n=8 (BE4 and YE1) or n=7 (Cas9 nickase). P values were calculated using the Mann-Whitney U test. **p<0.005; ***p<0.0005.

FIGS. 20A-20B show the HSV thymidine kinase resistance assay. FIG. 20A shows the fraction of dP-resistant colonies was calculated relative to number of E. coli plated on maintenance antibiotics. All constructs are in the deaminase-dCas9-UGI (BE2) architecture, except BE1 and BE1(E63A), both of which lack UGI, and the UGI only construct. Data are shown as individual data points and mean±SEM for n=3 bacterial colonies. FIG. 20B shows Sanger sequencing traces from four unique dP-resistant colonies that showed different C•G-to-T•A mutations that inactivate the integrated HSV thymidine kinase gene.

FIG. 21 shows HEK293T cell viability 48 or 72 hours post transfection as measured by luminescence using the CellTiter-Glo assay (Promega). Data are shown as individual data points and mean values±SEM for n=3 independent biological replicates.

FIGS. 22A-22F show Cas9-independent off-target editing by ABE. FIG. 22A shows the Rifampin assay for ABE (tadA-tadA*-dCas9) compared to BE2 (APOBEC1-dCas9-UGI) and BE1(E63A) (APOBEC1(E63A)-dCas9). The defective chloramphenicol acetyltransferase gene used to assess ABE on-target editing contained an inactivating G•C-to-A•T mutation rather than an inactivating T•A-to-C•G mutation. Both C•G-to-T•A and A•T-to-G•C mutations in the rpoB gene render E. coli resistant to rifampin (17). Data are shown as individual data points and mean values±SEM for n=3 bacterial colonies. Bars representing colonies in which off-target editing occurred are indicated with the * symbol. FIG. 22B shows HSV thymidine kinase assay for ABE compared to BE2 and BE1 (E63A). Data are shown as individual data points and mean values±SEM for n=3 bacterial colonies. FIG. 22C shows intracellular deamination of a co-transfected ssDNA oligonucleotides by ABEmax in HEK293T cells. Each data point represents the detected A-to-G conversion of a specific A in the oligonucleotide, and is the mean of n=3 biological replicates performed at different times. FIG. 22D shows on-target genomic DNA A•T-to-G•C editing controls at A₅ of the HEK2 locus. FIG. 22E shows off-target editing by ABEmax at R-loops created by dSaCas9 and one of six SaCas9 sgRNAs. FIG. 22F shows on-target editing controls at A₅ of the HEK2 locus. (Bars representing reads for which no editor was present are indicated with the * symbol.) In FIGS. 22D-22F, data are shown as individual data points and mean values±SEM for n=3 biological replicates performed at different times.

FIGS. 23A-23B show on-target and off-target editing profiles of CBEs. FIG. 23A shows the ratio of on-target control editing at the RNF2 locus to the average off-target editing across the six SaCas9 R loops tested. Each bar is the quotient of mean on-target control RNF2 editing/the mean off-target editing at all 18 cytosines tested in the orthogonal R-loop assay. Individual values used in these calculations are shown in FIGS. 2A-2D and FIGS. 8A-8D. FIG. 23B shows on-target editing across the six genomic loci for CBEs with low Cas9-independent off-target editing. Data are shown as individual data points and mean values±SEM for n=3 biological replicates performed at different times.

FIG. 24 shows the on-target controls for WGS samples. On-target sequencing of RNF2 locus of bulk populations after flow sorting is also shown.

FIGS. 25A-25B show the number of SNVs detected by WGS relative to the initial parent sample, separated by type of mutation. FIG. 25A shows the total number of SNVs of each type present in each sample. FIG. 25B shows a fraction of total SNPs in each sample that were a certain type of SNP. The fraction of total SNPs targeted for editing with the BE4 and YE1-BE4 editors are indicated with the “#” and “*” symbols, respectively. In both FIG. 25A and FIG. 25B, each dot represents the number of SNPs called in a clonal population of cells relative to the parent sample. Each clonal population was derived from a single GFP-positive cell that was isolated after flow sorting HEK293T cells transfected with a P2A-GFP construct of the designated editor. Lines indicate mean number of SNPs±SEM for n=8 (BE4 and YE1) or n=7 (nickase).

FIGS. 26A-26C show CBEs with minimal Cas9-independent off-target DNA editing show reduced levels of Cas9-independent RNA editing in HEK293T cells. FIG. 26A shows the average % C-to-U off-target RNA editing among cytosines within a transcript. Individual data points and mean values±SEM for n=3 biological replicates performed on different days are shown. Each replicate is the mean C-to-U editing across all of the cytosines of the specified transcript. FIG. 26B shows the number of cytosines with off-target RNA editing per transcript. Individual data points and mean values±SEM for n=3 biological replicates performed on different days are shown. Each replicate is the number of cytosines edited at a rate of 0.1% or higher within the designated transcript. FIG. 26C shows the editing of all 154 cytosines examined across the three transcripts. Each dot show the mean editing value of one specific cytosine, across three biological replicates performed on different days.

FIG. 27 shows CBE protein expression levels. Western blot analysis of HEK293T cell lysates 48 hours post transfection with plasmids encoding CBEs and an on-target sgRNA. Following membrane transfer, the top and bottom halves of the membrane were separated and processed separately (i.e. with different primary antibodies) but were imaged simultaneously. GAPDH was used as a loading control. Cell lysates were obtained from two biological replicates performed at different times.

FIG. 28 shows fluorescence-activated cell sorting (FACS) gating and data for a negative control (GFP-negative) HEK293T cell sample in order to subsequently gate for GFP-positive populations. Populations of cells gated for various events are indicated by symbols and shading.

FIG. 29 shows FACS gating and data for HEK293T cells transfected with a plasmid encoding YE1-P2A-GFP and a plasmid encoding an RNF2-targeting sgRNA. P4 shows gating for all GFP-positive cells, and P5 shows gating for the top ˜25% of GFP-positive cells. Populations of cells gated for various events are indicated by symbols and shading.

FIG. 30 shows FACS gating and data for HEK293T cells transfected with a plasmid encoding BE4-P2A-GFP and a plasmid encoding an RNF2-targeting sgRNA. Gates were maintained at the same locations as described above. Populations of cells gated for various events are indicated by symbols and shading.

FIG. 31 shows FACS gating and data for HEK293T cells transfected with a plasmid encoding Cas9(D10A)-P2A-GFP and a plasmid encoding an RNF2-targeting sgRNA. Gates were maintained at the same locations as described above. Populations of cells gated for various events are indicated by symbols and shading.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

“Base editing” is a genome editing technology that involves the conversion of a specific nucleic acid base (or nucleobase) into another at a targeted genomic locus. In certain embodiments, this can be achieved without requiring double-stranded DNA breaks (DSB). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g., typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation. See Komor, A. C., et al., Programmable editing of a target base in genomic DNA without double-stranded DNA cleavage. Nature 533, 420-424 (2016), which is incorporated by reference herein.

In principle, there are 12 possible base-to-base changes that may occur via individual or sequential use of transition (i.e., a purine-to-purine change or pyrimidine-to-pyrimidine change) or transversion (i.e., a purine-to-pyrimidine or pyrimidine-to-purine) editors. These include transition base editors such as the cytosine base editor (“CBE”), also known as a C-to-T base editor (or “CTBE”). This type of editor converts a C:G Watson-Crick nucleobase pair to a T:A Watson-Crick nucleobase pair. Because the corresponding Watson-Crick paired bases are also interchanged as a result of the conversion, this category of base editor may also be referred to as a guanine base editor (“GBE”) or G-to-A base editor (or “GABE”). Other transition base editors include the adenine base editor (or “ABE”), also known as an A-to-G base editor (“AGBE”). This type of editor converts an A:T Watson-Crick nucleobase pair to a G:C Watson-Crick nucleobase pair. Because the corresponding Watson-Crick paired bases are also interchanged as a result of the conversion, this category of base editor may also be referred to as a thymine base editor (or “TBE”) or T-to-G base editor (“TGBE”).

The term “base editors (BEs),” as used herein, refers to fusion proteins comprising protein domains from at least two proteins that are capable of editing nucleobase, and comprise the fusion proteins described herein. In some embodiments, the disclosed base editors comprise a nuclease-inactive Cas9 (dCas9) fused to a deaminase which binds a nucleic acid in a guide RNA-programmed manner via the generation of an R-loop but does not cleave the nucleic acid. For example, the dCas9 domain of a disclosed base editor may include both D10A and H840A mutations. In other embodiments, the disclosed base editors comprise a Cas9 nickase (nCas9) fused to a deaminase. The nCas9 domain of a disclosed base editor may include a D10A or an H840A mutation (which renders the Cas9 domain capable of cleaving only one strand of a nucleic acid duplex), as described in PCT/US2016/058344, filed on Oct. 22, 2016, and published as WO 2017/070632 on Apr. 27, 2017), which is incorporated herein by reference. The DNA cleavage domain of S. pyogenes Cas9 includes two subdomains, the HNH nuclease subdomain and the RuvC1 subdomain. The HNH subdomain cleaves the strand complementary to the gRNA (the “targeted strand,” or the strand at which editing or deamination occurs), whereas the RuvC1 subdomain cleaves the non-complementary strand containing the PAM sequence (the “non-targeted strand”, or the strand at which editing or deamination does not occur). The RuvC1 nCas9 mutant D10A generates a nick on the targeted strand, while the HNH nCas9 mutant H840A generates a nick on the non-targeted strand (see Jinek et al., Science 337:816-821(2012); Qi et al., Cell 28; 152(5):1173-83 (2013))

In some embodiments, the base editor comprises a Cas9 nickase fused to a deaminase, e.g., a deaminase which converts a cytosine nucleobase to a thymine. The term “base editors” encompasses the base editors described herein as well as any base editor known or described in the art at the time of this filing or developed in the future. Reference is made to Rees & Liu, Base editing: precision chemistry on the genome and transcriptome of living cells, Nat Rev Genet. 2018; 19(12):770-788 and Koblan et al., Nat Biotechnol. 2018; 36(9):843-846; as well as U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; International Publication No. WO 2018/027078, published Aug. 2, 2018; International Application No PCT/US2018/056146, filed Oct. 16, 2018, which published as Publication No. WO 2019/079347 on Apr. 25, 2019; International Application No PCT/US2019/033848, filed May 23, 2019, which published as Publication No. WO 2019/226593 on Nov. 28, 2019; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; International Publication No. WO 2019/023680, published Jan. 31, 2019; International Publication No. WO 2018/0176009, published Sep. 27, 2018; International Application No. PCT/US2019/47996, filed Aug. 23, 2019, which published as International Publication No. WO 2020/041751 on Feb. 27, 2020; International Publication No. WO 2020/051360, published Mar. 12, 2020; International Patent Publication No. WO 2020/102659, published May 22, 2020; International Publication No. WO 2020/086908, published Apr. 30, 2020; International Publication No. WO 2020/181180, published Sep. 10, 2020; International Publication No. WO 2020/214842, published Oct. 22, 2020; International Publication No. WO 2020/092453, published May 7, 2020 and International Patent Application No. PCT/US2020/033873, filed May 20, 2020, the contents of each of which are incorporated herein by reference in their entireties.

The term “Cas9” or “Cas9 nuclease” or “Cas9 domain” refers to a CRISPR associated protein 9, or variant thereof, and embraces any naturally occurring Cas9 from any organism, any naturally-occurring Cas9, any Cas9 homolog, ortholog, or paralog from any organism, and any variant of a Cas9, naturally-occurring or engineered. More broadly, a Cas9 protein, domain, or moiety is a type of nucleic acid programmable D/RNA binding protein (napR/DNAbp),” or more specifically, a “nucleic acid programmable DNA binding protein (napDNAbp)”. The term Cas9 is not meant to be limiting and may be referred to as a “Cas9 or variant thereof.” Exemplary Cas9 proteins are described herein and also described in the art. The present disclosure is unlimited with regard to the particular Cas9 that is employed in the base editors of the invention.

In some embodiments, proteins comprising Cas9 or fragments thereof are referred to as “Cas9 variants.” A Cas9 variant shares homology to Cas9, or a fragment thereof. Cas9 variants include functional fragments of Cas9. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to a wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

As used herein, the term “dCas9” refers to a nuclease-inactive Cas9 or nuclease-dead Cas9, or a variant thereof, and embraces any naturally occurring dCas9 from any organism, any naturally-occurring dCas9 equivalent, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and methods for making dCas9 proteins are further described herein and/or are described in the art and are incorporated herein by reference.

As used herein, the term “nCas9” or “Cas9 nickase” refers to a Cas9 or a variant thereof, which cleaves or nicks only one of the strands of a target cut site thereby introducing a nick in a double strand DNA molecule rather than creating a double strand break. This can be achieved by introducing appropriate mutations in a wild-type Cas9 which inactives one of the two endonuclease activities of the Cas9. Any suitable mutation which inactivates one Cas9 endonuclease activity but leaves the other intact is contemplated, such as one of D10A or H840A mutations in the wild-type Cas9 amino acid sequence may be used to form the nCas9. In various embodiments, the D10A mutation is used to form the nCas9 (e.g., SEQ ID NO: 215).

The term “circularly permuted Cas9” refers to a Cas9 protein, or variant thereof, that occurs as a circular permutant, whereby its N- and C-termini have been topically rearranged. Such circularly permuted Cas9 proteins (“CP-Cas9”), or variants thereof, retain the ability to bind DNA when complexed with a guide RNA (gRNA). See, Oakes et al., “Protein Engineering of Cas9 for enhanced function,” Methods Enzymol, 2014, 546: 491-511; Oakes et al., “CRISPR-Cas9 Circular Permutants as Programmable Scaffolds for Genome Modification,” Cell, Jan. 10, 2019, 176: 254-267; and International Publication No. WO 2020/041751, published Feb. 27, 2020, each of are incorporated herein by reference. The present disclosure contemplates any previously known CP-Cas9 or use a new CP-Cas9 so long as the resulting circularly permuted protein retains the ability to bind DNA when complexed with a gRNA.

“CRISPR” is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively constitute, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (mc), and a Cas9 protein. The tracrRNA serves as a guide for ribonuclease 3-aided processing of pre-crRNA. Subsequently, Cas9/crRNA/tracrRNA endonucleolytically cleaves linear or circular nucleic acid target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate embodiments of both the crRNA and tracrRNA into a single RNA species—the guide RNA. See, e.g., Jinek M., et al., Science 337:816-821 (2012), the entire contents of which is incorporated herein by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti J. J., et al., Proc. Natl. Acad. Sci. U.S.A. 98:4658-4663(2001); “CRISPR RNA maturation by trans-encoded small RNA and host factor RNase III.” Deltcheva E., et al., Nature 471:602-607 (2011); and “A programmable dual-RNA-guided DNA endonuclease in adaptive bacterial immunity.” Jinek M., et al., Science 337:816-821(2012), the entire contents of each of which are incorporated herein by reference). Cas9 orthologs have been described in various species, including, but not limited to, S. pyogenes, S. thermophiles, C. ulcerans, S. diphtheria, S. syrphidicola, P intermedia, S. taiwanense, S. iniae, B. baltica, P torquis, S. thermophilus, L. innocua, C. jejuni, G. thermodenitrificans and N. meningitidis. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. The tracrRNA of the system is complementary (fully or partially) to the tracr mate sequence present on the guide RNA.

The term “effective amount,” as used herein, refers to an amount of a biologically active agent that is sufficient to elicit a desired biological response. For example, in some embodiments, an effective amount of a base editor may refer to the amount of the base editor that is sufficient to edit a target site nucleotide sequence, e.g., a genome. In some embodiments, an effective amount of a base editor provided herein, e.g., of a base editor comprising a Cas9 nickase domain and a nucleobase modification domain (e.g., a deaminase domain) may refer to the amount of the base editor that is sufficient to induce editing of a target site specifically bound and edited by the base editor. In some embodiments, an effective amount of a base editor provided herein may refer to the amount of the base editor sufficient to induce editing having the following characteristics: >50% product purity, <5% indels over regions immediately surrounding the target sequence, and/or an editing window of 2-8 nucleotides. In other embodiments, an effective amount of a base editor may refer to the amount of the base editor sufficient to induce editing of >45% product purity, <10% indels, a ratio of intended point mutations to indels that is at least 5:1, and/or an editing window of 2-10 nucleotides. As will be appreciated by the skilled artisan, the effective amount of an agent, e.g., a base editor, a nuclease, a deaminase, a hybrid protein, a complex of a protein and a polynucleotide, or a polynucleotide (e.g., gRNA), may vary depending on various factors, such as, for example, on the desired biological response, e.g., on the specific allele, genome, or target site to be edited, on the target cell or tissue (i.e., the cell or tissue to be edited), and on the agent being used.

The term “off-target editing,” as used herein, refers to the introduction of unintended modifications (e.g., deaminations) to nucleotides (e.g. cytosine) in a sequence outside the canonical base editor binding window (i.e., from one protospacer position to another, typically 2 to 8 nucleotides long). Off-target editing can result from weak or non-specific binding of the gRNA sequence to the target sequence. Off-target editing can also result from intrinsic association of the nucleotide modification domain (e.g. deaminase domain) of a base editor to nucleobases in loci unrelated to the target sequence.

The term “Cas9-dependent off-target editing” refers to the introduction of unintended modifications that result from weak or non-specific binding of a Cas9-gRNA complex (e.g., a complex between a gRNA and the base editor's Cas9 domain) to nucleic acid sites that have fairly high (e.g. more than 60%, or having fewer than 6 mismatches relative to) sequence identity to a target sequence. In contrast, the term “Cas9-independent off-target editing” refers to the introduction of unintended modifications that result from weak associations of a base editor (e.g., the nucleotide modification domain) to nucleic acid sites that do not have high sequence identity (about 60% or less, or having 6-8 or more mismatches relative to) to a target sequence. Because these associations occur independent of any hybridization between the Cas9-gRNA complex and the relevant nucleic acid site, they are referred to as “Cas9-independent.”

The term “on-target editing,” as used herein, refers to the introduction of intended modifications (e.g., deaminations) to nucleotides (e.g., cytosine) in a target sequence, such as using the base editors described herein.

The terms “on-target editing frequency” and “on-target editing efficiency”, as used herein, refers to the number or proportion of intended base pairs that are edited. For example, if a base editor edits 10% of the base pairs that it is intended to target (e.g., within a cell or within a population of cells), then the base editor can be described as being 10% efficient. Some aspects of editing efficiency embrace the modification (e.g., deamination) of a specific nucleotide within DNA, without generating a large number or percentage of insertions or deletions (i.e., indels). It is generally accepted that editing while generating less than 5% indels over regions immediately surrounding the target sequence (as measured over total target nucleotide substrates) constitutes high editing efficiency. The generation of more than 20% indels is generally accepted as poor or low editing efficiency.

The term “off-target editing frequency,” as used herein, refers to the number or proportion of unintended base pairs that are edited. On-target and off-target editing frequencies may be measured by the methods and assays described herein, further in view of techniques known in the art, including high-throughput sequencing reads. As used herein, high-throughput sequencing involves the hybridization of nucleic acid primers (e.g., DNA primers) with complementarity to nucleic acid (e.g., DNA) regions just upstream or downstream of the target sequence or off-target sequence of interest. Because the DNA target sequence and the Cas9-independent off-target sequences are known a priori in the methods disclosed herein, nucleic acid primers with sufficient complementarity to regions upstream or downstream of the target sequence and Cas9-independent off-target sequences of interest may be designed using techniques known in the art, such as the PhusionU PCR kit (Life Technologies), Phusion HS II kit (Life Technologies), and Illumina MiSeq kit. Since many of the Cas9-dependent off-target sites have high sequence identity to the target site of interest, nucleic acid primers with sufficient complementarity to regions upstream or downstream of the Cas9-dependent off-target site may likewise be designed using techniques and kits known in the art. These kits make use of polymerase chain reaction (PCR) amplification, which produces amplicons as intermediate products. The target and off-target sequences may comprise genomic loci that further comprise protospacers and PAMs. Accordingly, the term “amplicons,” as used herein, may refer to nucleic acid molecules that constitute the aggregates of genomic loci, protospacers and PAMs. High-throughput sequencing techniques used herein may further include Sanger sequencing and/or whole genome sequencing (WGS).

The term “linker,” as used herein, refers to a chemical group or a molecule linking two molecules or domains, e.g., a Cas9 domain and a deaminase domain. In some embodiments, a linker joins a Cas9 nickase domain and cytidine deaminase domain of the disclosed base editors. In some embodiments, a linker joins a cytidine deaminase domain and a UGI domain, and/or joins each of one or more UGI domains, within the disclosed base editors. Typically, the linker is positioned between, or flanked by, two groups, molecules, or other domains and connected to each one via a covalent bond, thus connecting the two. In some embodiments, the linker is an amino acid or a plurality of amino acids (e.g., a peptide or protein). In some embodiments, the linker is an organic molecule, group, polymer (e.g., polyethylene and polyethylene glycol), or chemical domain. Chemical domains include, but are not limited to, amide, urea, carbamate, carbonate, ester, ketone, acetal, ketal, phosphoramidite, hydrazone, imine, oxime, disulfide, silyl, hydrazine, hydrazone, thiol, imidazole, ether, thioether, carbon-carbon bond, carbon-heteroatom bond, and azo domains. The linker may comprise a moiety derived from a click chemistry reaction (e.g., triazole, diazole, diazine, sulfide bond, maleimide ring, succinimide ring, ester, amide).

In some embodiments, the linker is 3-200 amino acids in length, for example, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In certain embodiments, the linker is 9 amino acids in length. In certain embodiments, the linker is 32 amino acids in length.

The term “mutation,” as used herein, refers to a substitution of a residue within a sequence, e.g., a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome which results from a mutation in the gene for the connective tissue protein fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Alternatively the mutation could lead to overexpression of one or more genes involved in control of the cell cycle, thus leading to uncontrolled cell division and hence to cancer. Because of their nature, gain-of-function mutations are usually dominant.

The terms “non-naturally occurring” or “engineered” are used interchangeably and indicate the involvement of the hand of man. The terms, when referring to nucleic acid molecules or polypeptides (e.g., Cas9 or deaminases) mean that the nucleic acid molecule or the polypeptide is at least substantially free from at least one other component with which they are naturally associated in nature and/or as found in nature (e.g., an amino acid sequence not found in nature).

The term “nucleic acid,” as used herein, refers to RNA as well as single- and/or double-stranded DNA. Nucleic acids may be naturally occurring, for example, in the context of a genome, a transcript, an mRNA, tRNA, rRNA, siRNA, snRNA, plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein to modify guide RNA molecules, the term “backbone” refers to the component of the guide RNA that comprises the core region, also known as the crRNA/tracrRNA. The backbone is separate from the guide sequence, or spacer, region of the guide RNA, which has complementarity to a protospacer of a nucleic acid molecule.

The term “nucleic acid programmable D/RNA binding protein (napR/DNAbp)” refers to any protein that may associate (e.g., form a complex) with one or more nucleic acid molecules (i.e., which may broadly be referred to as a “napR/DNAbp-programming nucleic acid molecule” and includes, for example, guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term “napR/DNAbp” embraces napDNAbps, such as CRISPR Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system, also known as Cas13a), C2c3 (a type V CRISPR-Cas system, also known as Cas12c), dCas9, GeoCas9, CjCas9, Cas12a (e.g., LbCas12a, AsCas12a, CeCas12a and MbCas12a), Cas12b, Cas12g, Cas12h, Cas12i, Cas13b, Cas13c, Cas13d, Cas14, Csn2, Argonaute (Ago), nCas9, xCas9, SpCas9-NG, Cas9-KKH, SmacCas9, Spy-macCas9, an SpCas9-NRRH, an SpCas9-NRCH, and an SpCas9-NRTH, and circularly permuted Cas9 domains, such as CP1012, CP1028, CP1041, CP1249, and CP1300. Additional napDNAbp Cas equivalents include Cas3 and CasΦ. Additional Cas-equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding protein (napDNAbp) that may be used in connection with this invention are not limited to CRISPR-Cas systems. The invention embraces any such programmable protein, such as the Argonaute protein from Natronobacterium gregoryi (NgAgo), which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao et al., DNA-guided genome editing using the Natronobacterium gregoryi Argonaute. Nature Biotechnology 2016; 34(7):768-73, which is incorporated herein by reference.

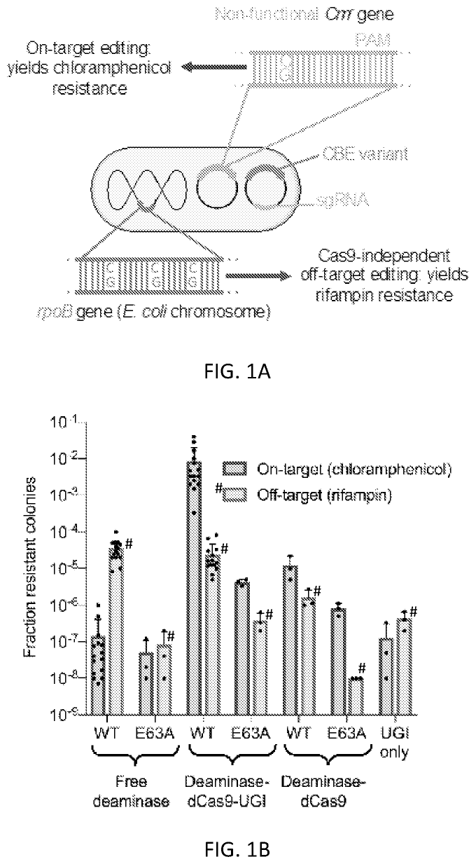

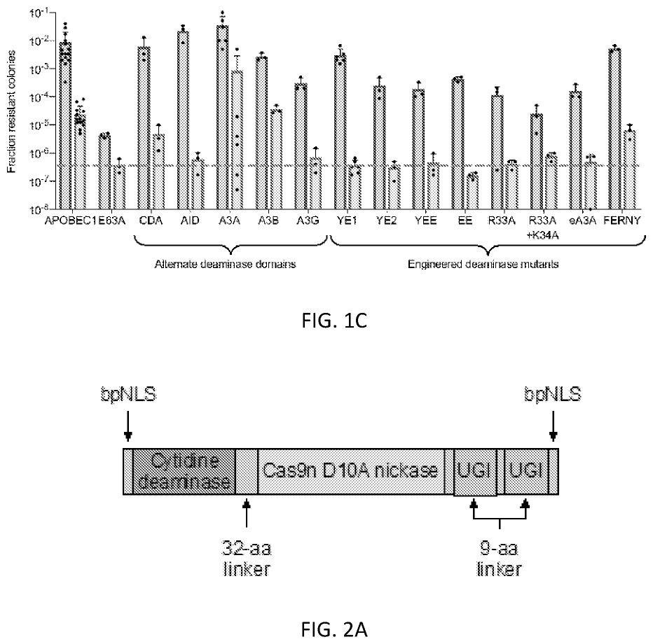

In some embodiments, the napR/DNAbp is a RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821(2012), the entire contents of which is incorporated herein by reference. Other examples of gRNAs (e.g., those including domain 2) can be found in U.S. Pat. No. 9,340,799, entitled “mRNA-Sensing Switchable gRNAs,” and International Patent Application No. PCT/US2014/054247, filed Sep. 6, 2013, published as WO 2015/035136 on Mar. 12, 2015, and entitled “Delivery System For Functional Nucleases,” the entire contents of each are incorporated herein by reference. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J. J. et al., Proc. Natl. Acad. Sci. U.S.A. 98:4658-4663(2001); “CRISPR RNA maturation by trans-encoded small RNA and host factor RNase III.” Deltcheva E. et al., Nature 471:602-607 (2011); and Jinek M. et al., “A programmable dual-RNA-guided DNA endonuclease in adaptive bacterial immunity.” Science 337:816-821 (2012), each of which is incorporated herein by reference.

The napR/DNAbp nucleases (e.g., Cas9) use RNA:DNA hybridization to target DNA cleavage sites, these proteins are able to be targeted, in principle, to any sequence specified by the guide RNA. Methods of using napR/DNAbp nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al., Genome engineering in Saccharomyces cerevisiae using CRISPR-Cas systems. Nucleic Acid Res. (2013); Jiang, W. et al. RNA-guided editing of bacterial genomes using CRISPR-Cas systems. Nature Biotechnology 31, 233-239 (2013); the entire contents of each of which are incorporated herein by reference).

The term “napR/DNAbp-programming nucleic acid molecule” or equivalently “guide sequence” refers the one or more nucleic acid molecules which associate with and direct or otherwise program a napR/DNAbp protein to localize to a specific target nucleotide sequence (e.g., a gene locus of a genome) that is complementary to the one or more nucleic acid molecules (or a portion or region thereof) associated with the protein, thereby causing the napR/DNAbp protein to bind to the nucleotide sequence at the specific target site. A non-limiting example is a guide RNA of a Cas protein of a CRISPR-Cas genome editing system.

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences can be of any size and composition, for example, more than 25, 25, 15, 12, 10, 8, 7, 6, 5, or 4 amino acids, but will preferably comprise at least a four to eight amino acid sequence known to function as a nuclear localization signal (NLS). NLS sequences are described in Plank et al., International PCT Application PCT/EP2000/011690, filed Nov. 23, 2000, published as WO/2001/038547 on May 31, 2001, the contents of which are incorporated herein by reference

The term, as used herein, “nucleotide modification domain” embraces any protein, enzyme, or polypeptide (or variant thereof) which is capable of modifying, substituting, replacing, or exchanging a DNA or RNA molecule (e.g. a DNA or RNA nucleobase). Nucleotide modification domains may be naturally occurring, or may be engineered. For example, a nucleotide modification domain can include one or more DNA repair enzymes, for example, and an enzyme or protein involved in base excision repair (BER), nucleotide excision repair (NER), homology-dependnent recombinational repair (HR), non-homologous end-joining repair (NHEJ), microhomology end-joining repair (MMEJ), mismatch repair (MMR), direct reversal repair, or other known DNA repair pathway. A nucleotide modification domain can have one or more types of enzymatic activities, including, but not limited to, endonuclease activity, polymerase activity, ligase activity, replication activity, and proofreading activity. Nucleotide modification domains include DNA or RNA-modifying enzymes and/or DNA or RNA-displacing enzymes, such as base exchange enzymes, deaminases, which covalently modify nucleobases leading in some cases to mutagenic corrections by way of normal cellular DNA repair and replication processes. Exemplary nucleotide modification domains include, but are not limited to, a deaminase, a nuclease, a nickase, a recombinase, a methyltransferase, a methylase, an acetylase, an acetyltransferase, a transcriptional activator, or a transcriptional repressor domain. In some embodiments the nucleotide modification domain is a deaminase domain (e.g., APOBEC1, AID or CDA).

As used herein, the terms “oligonucleotide” and “polynucleotide” can be used interchangeably to refer to a polymer of nucleotides (e.g., a string of at least three nucleotides).

The term “promoter” is art-recognized and refers to a nucleic acid molecule with a sequence recognized by the cellular transcription machinery and able to initiate transcription of a downstream gene. A promoter can be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect. In various embodiments, the specification provides vectors with appropriate promoters for driving expression of the nucleic acid sequences encoding the base editor base editors (or one or more individual components thereof).

As used herein, the term “protospacer” refers to the sequence (˜20 bp) in DNA adjacent to the PAM (protospacer adjacent motif) sequence which is complementary to the guide, or “spacer,” sequence of the guide RNA. The guide RNA anneals to the protospacer sequence on the target DNA (specifically, one strand thereof, i.e, the “target strand” versus the “non-target strand” of the target sequence). In order for Cas9 to function it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the target sequence in the genomic DNA, on the non-target strand. The skilled person will appreciate that the literature in the state of the art sometimes refers to the “protospacer” as the ˜20-nt target-specific guide sequence on the guide RNA itself, rather than referring to it as a “spacer.” The context of the description surrounding the appearance of either “protospacer” or “spacer” will help inform the reader as to whether the term is reference to the gRNA or the DNA target. Both usages of these terms are acceptable as the state of the art uses both terms in each of these ways.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, engineered, or synthetic, or any combination thereof. In some embodiments, the protein or polypeptide is a fusion protein.

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “product purity,” as used herein, refers to the percentage of desired products over total products of a base editing reaction. For instance, product purity of a CBE may be measured as the percentage of total edited sequencing reads (reads in which a target C has been converted to a different base) in which the target C is edited to a T, over a portion of interest of the nucleic acid. Product purity embraces the absence of indels, as well as the desired product of a base conversion.

The term “R-loop” refers to a triplex structure wherein the two strands of a double-stranded DNA are separated for a stretch of nucleotides and held apart by a single-stranded RNA molecule (e.g., gRNA). R-loop formation may be induced by the hybridization of a gRNA having complementarity to the DNA, in association with a napDNAbp protein or domain (e.g., Cas9). Two R-loops are referred to as “orthogonal” when the mechanisms (e.g., napDNAbp-gRNA complexes) that generate their formation function independently of one another.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to a naturally occurring sequence.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent (e.g., mouse, rat). In some embodiments, the subject is a sheep, a goat, a cattle, a cat, or a dog. In some embodiments, the subject is a vertebrate. In some embodiments, the subject is an amphibian, a reptile, a fish, an insect (e.g., fly), or a nematode. In some embodiments, the subject is a research animal. In some embodiments, the subject is an experimental organism. In some embodiments, the subject is a plant. In some embodiments, the subject is genetically engineered, e.g., a genetically engineered non-human subject. The subject may be of either sex and at any stage of development. In some embodiments, the subject is a microorganism, such as a bacteria.

The terms “target sequence” and “target site” refer to a sequence within a nucleic acid molecule that is edited by a base editor (e.g., a base editor as provided herein). The target site contains a protospacer sequence within a nucleic acid molecule to which a complex of the base editor and the guide RNA binds. The protospacer sequence must be complementary to the gRNA. The target sequence must also contain a “protospacer-adjacent motif” (PAM) at the 3′-end of the protospacer. Base editing typically requires a PAM to be positioned approximately 13-17 nucleotides from a target base pair and some forms of homology-directed repair, which are most efficient when DNA cleavage occurs ˜ 10-20 base pairs away from a desired alteration. To address this limitation, researchers have harnessed natural CRISPR nucleases with different PAM requirements and engineered existing systems to accept variants of naturally recognized PAMs. Other natural CRISPR nucleases shown to function efficiently in mammalian cells include Staphylococcus aureus Cas9 (SaCas9), Acidaminococcus sp. Cpf1 (AsCpf1), Lachnospiraceae bacterium Cpf1, Campylobacter jejuni Cas9, Streptococcus thermophilus Cas9, and Neisseria meningitides Cas9.

The term “vector,” as used herein, may refer to a nucleic acid that has been modified to encode a base editor and/or one or more gRNAs. Exemplary vectors may also encode one or more isolated napDNAbps, such as isolated Cas9 proteins (e.g., nuclease inactive Cas9 proteins). Exemplary suitable vectors include viral vectors, such as retroviral vectors and AAV vectors, and plasmids.

The term “viral vector,” as used herein, refers to a nucleic acid comprising a viral genome that, when introduced into a suitable host cell, can be replicated and packaged into viral particles able to transfer the viral genome into a host cell, e.g. by integration of the viral genome into the host cell genome. In some embodiments, the viral vector is an adeno-associated virus (AAV) vector.

The term “viral particle,” as used herein, refers to a viral genome, for example, a DNA or RNA genome, that is associated with a coat of a viral protein or proteins, and, in some cases, with an envelope of lipids. For example, a phage particle comprises a phage genome packaged into a protein encoded by the wild type phage genome.

The terms “treatment,” “treat,” and “treating,” refer to a clinical intervention aimed to reverse, alleviate, delay the onset of, or inhibit the progress of a disease, disorder, or condition, or one or more symptoms thereof, as described herein. As used herein, the terms “treatment,” “treat,” and “treating” refer to a clinical intervention aimed to reverse, alleviate, delay the onset of, or inhibit the progress of a disease, disorder, or condition, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their prevention or recurrence.

As used herein, the term “variant” refers to a protein having characteristics that deviate from what occurs in nature, e.g., a “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant nucleotide modification domain is a nucleotide modification domain comprising one or more changes in amino acid residues of a deaminase, as compared to the wild type amino acid sequences thereof. These changes include chemical modifications, including substitutions of different amino acid residues, as well as truncations. This term embraces functional fragments of the wild type amino acid sequence.

The level or degree of which the property is retained may be reduced relative to the wild type protein but is typically the same or similar in kind. Generally, variants are overall very similar, and in many regions, identical to the amino acid sequence of the protein described herein. A skilled artisan will appreciate how to make and use variants that maintain all, or at least some, of a functional ability or property.

The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein (e.g., Cas9 protein, base editor, and base editor protein). Further polypeptides encompassed by the invention are polypeptides encoded by polynucleotides which hybridize to the complement of a nucleic acid molecule encoding a protein such as a Cas9 protein under stringent hybridization conditions (e.g., hybridization to filter bound DNA in 6× sodium chloride/sodium citrate (SSC) at about 45 degrees Celsius, followed by one or more washes in 0.2.times.SSC, 0.1% SDS at about 50-65 degrees Celsius), under highly stringent conditions (e.g., hybridization to filter bound DNA in 6× sodium chloride/Sodium citrate (SSC) at about 45 degrees Celsius, followed by one or more washes in 0.1×SSC, 0.2% SDS at about 68 degrees Celsius), or under other stringent hybridization conditions which are known to those of skill in the art (see, for example, Ausubel, F. M. et al., eds., 1989 Current Protocol in Molecular Biology, Green publishing associates, Inc., and John Wiley & Sons Inc., New York, at pp. 6.3.1-6.3.6 and 2.10.3).

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a protein such as a Cas9 protein, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence

As used herein the term “wild type” is a term of the art understood by skilled persons and means the typical form of an organism, strain, gene, or characteristic as it occurs in nature as distinguished from mutant, engineered, or variant forms.

### Detailed Description of Certain Embodiments

Provided herein are novel assays for determining the off-target effects (e.g., off-target editing frequencies and indels) for base editors. Also provided herein are novel base editor proteins and constructs encoding these base editors, wherein the base editors have reduced Cas9-independent off-target editing frequencies while maintaining high on-target editing efficiencies. Further provided are methods of contacting these base editors with a nucleic acid molecule and obtaining low off-target editing frequencies.

The function and advantage of these and other embodiments of the present disclosure will be more fully understood from the Examples below. The following Examples are intended to illustrate the benefits of the present disclosure and to describe particular embodiments, but are not intended to exemplify the full scope of the disclosure. Accordingly, it will be understood that the Examples are not meant to limit the scope of the disclosure.

### Assays and Systems for Measuring Off-Target Frequencies

In some aspects, provided herein are methods for evaluating the off-target effects of a base editor. In various embodiments, these methods are designed for determining the off-target editing frequencies of napDNAbp domain-independent (e.g., Cas9-independent) (or gRNA-independent) off-target editing events. Editing events may comprise deamination events of a cytosine (or adenine) base by a CBE (or an adenine base editor, or ABE). Off-target deamination events that are dependent on the napDNAbp-guide RNA complex tend to be in sequences that have high sequence identity (e.g., greater than 60% sequence identity) to the target sequence. These types of events arise because of imperfect hybridization of the napDNAbp-guide RNA complex to sequences that share identity with the target sequence. In contrast, off-target events that occur independently of the napDNAbp-guide RNA complex arise as a result of stochastic binding of the base editor to DNA sequences (often sequences that do not share high sequence identity with the target sequence) due to an intrinsic affinity of the base editor of the nucleobase modification domain (e.g., the deaminase domain) of the base editor with DNA. NapDNAbp-independent (e.g., Cas9-independent) editing events arise in particular when the base editor is overexpressed in the system under evaluation, such as a cell or a subject.

Accordingly, in some embodiments, the present disclosure provides methods of determining the off-target editing frequency of a base editor comprising: (a) contacting a nucleic acid molecule comprising a target sequence, with a first complex. The first complex comprises (i) a base editor comprising a napDNAbp domain, and (ii) a first guide RNA that is engineered to bind to the napDNAbp domain of the base editor, wherein the first guide RNA comprises a first sequence of at least 10 contiguous nucleotides (i.e., a first guide sequence) that is complementary to the target sequence; (b) contacting the nucleic acid molecule with a second complex, wherein the second complex comprises (iii) a first nuclease inactive napDNAbp (e.g., a dead Cas9 (dCas9)) protein, and (iv) a second guide RNA that is engineered to bind to the first nuclease inactive napDNAbp protein, wherein the second guide RNA comprises a second sequence of at least 10 contiguous nucleotides (i.e., a second guide sequence) that is complementary to a third sequence (i.e., a known off-target sequence), whereby the first complex and second complex generate two or more R-loops in the nucleic acid molecule; and (c) sequencing at least a portion of the target sequence and/or at least a portion of the nucleic acid molecule comprising the third sequence. This sequencing step is performed to quantify the number of modified (i.e. non-wild-type) sequencing reads at both the target sequence and the third sequence, which would indicate on-target and off-target editing, respectively. The target sequence and third sequence each contain protospacers of 10-30 nucleotides that are complementary to the guide sequences of the first guide RNA and second guide RNA, respectively. In certain embodiments, these protospacers contain 20 nucleotides.

In some embodiments, the disclosed methods comprise contacting the nucleic acid molecule with additional complexes of isolated nuclease inactive napDNAbp protein in association with a gRNA. These complexes may be identical or essentially identical to each other, in that they are associated with identical or nearly identical gRNAs that have complementarity to the same off-target sequence. Any one of these complexes may be distinct or essentially identical to the second complex. The second and third guide RNA may share 100% sequence identity in the guide sequence of the guide RNA. The second and third guide RNA may share at least 95%, 98%, 98.5%, or 100% sequence identity in the backbone of the guide RNA sequence. In certain embodiments, the second and third guide RNA share 100% identity, or are the same. Likewise, in certain embodiments, the first nuclease inactive napDNAbp protein and the second nuclease inactive napDNAbp share 100% identity, or are the same.

The disclosed methods comprise the use of a third, fourth, fifth, sixth, seventh, eighth, ninth, and/or tenth complex. Accordingly, the methods may further comprise a step of contacting the nucleic acid molecule with a third, fourth, fifth, and/or sixth complex, wherein each of the third, fourth, fifth, and/or sixth complexes comprises (v) a second (isolated) nuclease inactive napDNAbp protein, and (vi) a third guide RNA that is engineered to bind to the second nuclease inactive napDNAbp protein, wherein the third guide RNA comprises a fourth sequence of at least 10 contiguous nucleotides (i.e., a guide sequence) that is complementary to the third sequence (off-target sequence). In particular embodiments, six total complexes of napDNAbp protein-gRNA are used. R-loops are generated by a nuclease inactive napDNAbp protein (e.g., a dead Cas9) and one or more guide RNAs (e.g., sgRNAs) that is engineered to bind the napDNAbp protein (e.g., a Cas9 derived from S. aureus). Guide RNAs are engineered to bind particular napDNAbp's (e.g., Cas9 proteins from different species) by modifying the backbone of the RNA to be specific for the binding pockets of the particular napDNAbp.

In some embodiments, the nucleic acid molecule is subsequently sequenced (e.g., through high-throughput sequencing) at loci comprising the third sequence to quantify the number of modified sequencing reads at this sequence.

The nuclease inactive napDNAbp protein of the described complexes is an isolated protein, i.e., it does not exist as a domain of a base editor or other fusion protein. In some embodiments, the nuclease inactive napDNAbp protein of any of the described complexes is a dead Cas9 (dCas9) protein. Accordingly, in some embodiments, the second complex comprises a first dCas9 protein, and the third and subsequent complexes each comprise a second dCas9 protein. In some embodiments, the nuclease inactive napDNAbp protein of any of the described complexes is a dead Cas9 protein from S. aureus. In some embodiments, the nuclease inactive napDNAbp protein is a dead Cas9 protein from S. pyogenes.

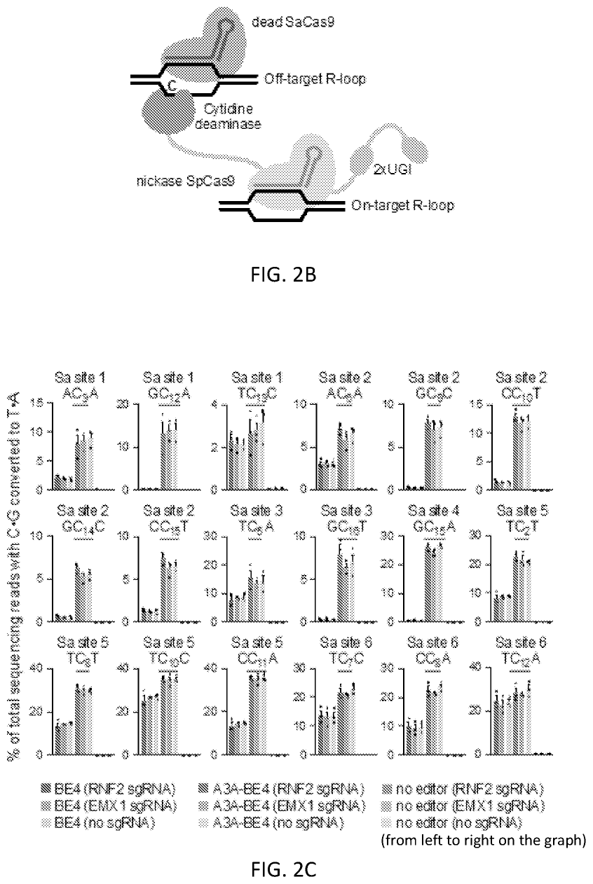

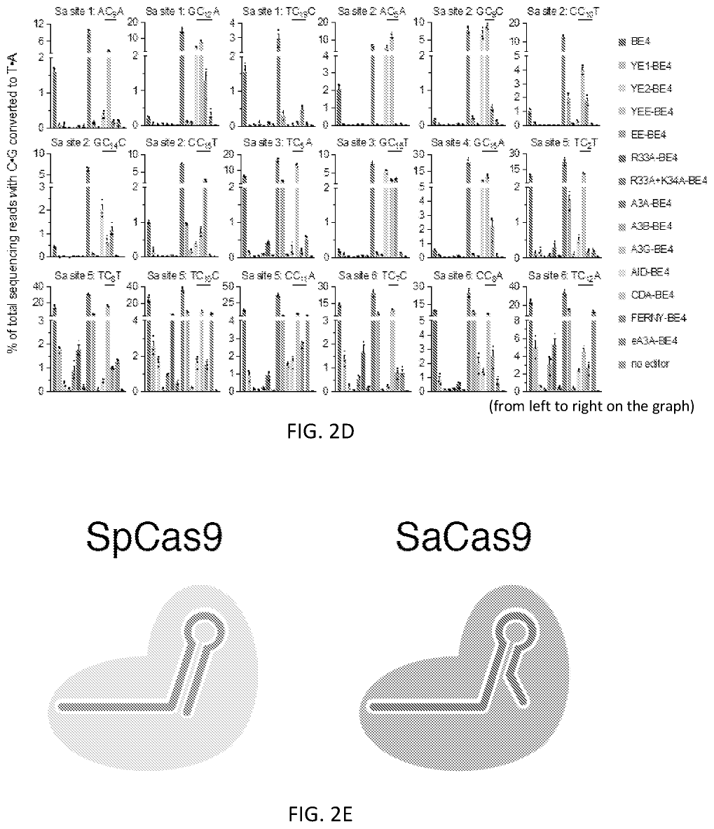

In some embodiments, the napDNAbp domain of the base editor is a Cas9 domain. The Cas9 domain of the base editor may comprise a wild-type Cas9. In some embodiments, the Cas9 domain of the base editor comprises a Cas9 nickase. In some embodiments, the Cas9 domain of the base editor comprises a dead Cas9. In some embodiments, the Cas9 domain is derived from a first bacterial species. In some embodiments, the first dCas9 protein and the second dCas9 protein are derived from a second bacterial species. In certain embodiments, the first bacterial species is S. pyogenes, and/or the second bacterial species is S. aureus. In other embodiments, the first bacterial species is S. aureus, and/or the second bacterial species is S. pyogenes. By taking advantage of the specificity of the backbones of engineered guide RNAs to particular Cas9 proteins, Cas9 proteins from different species may be used as orthogonal DNA-binding proteins, such that one may precisely direct a Cas9 protein to one of several off-target sites of interest by providing an orthogonal gRNAs that will be recognize only by a Cas9 protein of a single species (see FIG. 2E). For instance, in the disclosed methods, the base editor's Cas9 domain (e.g., Cas9 nickase domain) derived from a first species is directed to the on-target site, while the first dCas9 protein and the second dCas9 protein, which are derived from a second species, are directed to known off-target sites. Each of these isolated dCas9 proteins creates an “orthogonal R-loop” at the off-target sites, which serves as an ideal substrate for the measurement of off-target editing at a particular locus in lieu of measuring off-target editing across the whole genome. See FIG. 2B.

As described above, “R-loops” refer to triplex structures that arise when a single-stranded gRNA molecule “invades” and pulls apart the strands of a double-stranded DNA molecule and hybridizes (completely or partially) to one of the two strands. The R-loops may be induced by the napDNAbp domain of the base editor, the first and/or second the nuclease inactive napDNAbp protein, or both. In various embodiments, the R-loops are induced by the napDNAbp domain of the base editor, the first nuclease inactive napDNAbp protein and the second the nuclease inactive napDNAbp protein. The R-loop induced by the napDNAbp domain of the base editor may be referred to as an “on-target R-loop,” and the R-loop(s) induced by the nuclease inactive napDNAbp proteins may be referred to as an “off-target R-loops”.

The base editor used in the disclosed methods may be a cytosine base editor (CBE). CBEs enzymatically deaminate a cytosine nucleobase of a C:G nucleobase pair to a uracil. Accordingly, disclosed are methods designed for determining the off-target deamination frequencies of Cas9-independent (or gRNA-independent) off-target editing events of CBEs.

In other embodiments, the base editor of the disclosed methods may comprise an adenine base editor (ABE). In other embodiments, the base editor may comprise a transversion base editor, such as a C-to-G base editor (or “CGBE”), a G-to-T base editor (or “GTBE”), an A-to-T base editor (or “ATBE”), or an A-to-C base editor (or “ACBE”).

In order to evaluate Cas9-independent deamination events, the off-target sequence of the disclosed methods comprises an off-target site that is unrelated to the target site. Accordingly, the third sequence comprises a protospacer sequence that has about 70% or less sequence identity to the target sequence. The third sequence may comprise a protospacer sequence that has about 60% or less sequence identity to the target sequence. In other embodiments, the third sequence may comprise a protospacer sequence that has about 55% or less, 50% or less, 45% or less, 40% or less, 35% or less, or 30% or less sequence identity to the target sequence. The third sequence may comprise a protospacer sequence that differs from the protospacer of the target sequence in 5, 6, 7, 8, 9, 10, 11, 12, 13, 14, 15, or greater than 15 nucleotide positions (or has 5, 6, 7, 8, 9, 10, 11, 12, 13, 14, 15 or greater than 15 “mismatches” relative to the target sequence). In certain embodiments, the protospacer of the third sequence differs from the protospacer of the target sequence in 6 or more, 7 or more, or 8 or more nucleotide positions. Tsai, S. Q. et al. Nat. Biotechnol. 33, 187-197 (2015), and Komor, A. C. et al. Nature 533, 420-424 (2016), each of which is incorporated herein by reference, detected Cas9-dependent off-target DNA cleavage events at sites that shared at least 70% identity to the on-target sequence. In the experiments described in these publications, the number of nucleotide position mismatches between an on-target protospacer and a detected Cas9-dependent off-target protospacer, each having lengths of 20 nucleotides, was six mismatches. The target and off-target sequences of the disclosure may each have a length of 20-25 nucleotides, 25-35 nucleotides, 35-45 nucleotides, or more than 45 nucleotides.

In some embodiments, the first gRNA may comprise a first sequence of at least 15, at least 20, or more than 20 contiguous nucleotides that is complementary to the target sequence. In some embodiments, the second and/or third gRNAs may comprise a second sequence of at least 15, at least 20, or more than 20 contiguous nucleotides that is complementary to the third sequence.

In some embodiments, the first gRNA may comprise a plurality of unique gRNAs, wherein each comprises a sequence of at least 10 contiguous nucleotides that is complementary to one or more target sequences in the nucleic acid molecule. In some embodiments, the disclosed methods may detect substantially no difference in the degree of off-target editing when one or more unique on-target gRNAs are utilized because the off-target deamination events are gRNA sequence-independent (see, e.g., FIG. 2C).

The target sequence and third sequence may be distant from or proximal to one another. In some embodiments, the target sequence and the third sequence are within about 1000 nucleotides, 500 nucleotides, about 400 nucleotides, about 300 nucleotides, about 200 nucleotides, about 150 nucleotides, about 120 nucleotides, about 100 nucleotides, about 90 nucleotides, or about 75 nucleotides of one another. In some embodiments, the target sequence and the third sequence are about 0.01 m, 0.05 m, 0.1 m, 0.25 m, 0.5 m, 0.75 m, 1 m, or more than 1 m apart in three-dimensional space in the nucleus. The target sequence and third sequence may be on separate chromosomes.

The target sequence and the third sequence may be comprised within the genome of an organism. In some embodiments, the target sequence comprises a C:G nucleobase pair. In other embodiments, the target sequences comprises an A:T nucleobase pair, a T:A nucleobase pair, or a G:C nucleobase pair.

The step of contacting may further comprise the step of administering to (or transfecting) the cell one or more nucleic acid vectors encoding the base editor, the first gRNA, the first nuclease inactive napDNAbp protein, and the second gRNA. In some embodiments, the base editor and gRNA are administered as a protein:RNA complex, such as a ribonucleoprotein complex. In some embodiments, the step of contacting comprises further transfecting the cell with one or more plasmids encoding the second nuclease inactive napDNAbp protein and the third gRNA. The step of transfecting may be performed using lipofection, nucleofection, or electroporation. The nucleic acid vectors may comprise plasmids.

In some embodiments, the step of sequencing comprises performing high-throughput sequencing. High-throughput sequencing methods are known in the art.

In some embodiments, the disclosed methods of off-target effects evaluation yield off-target editing (e.g. off-target deamination) frequencies of less than 1.5% (such as less than 1.25%, less than 1.0%, less than 0.75%, or less than 0.5%) for one or more base editors (e.g., a CBE) under evaluation. The disclosed methods may further yield on-target editing efficiencies of greater than 50% (such as greater than 60%, greater than 70%, or greater than 85%) at the target nucleobase pair for one or more base editors under evaluation. These methods may yield an on-target editing efficiency of greater than 50% and a frequency of off-target editing of less than 1.5% for one or more base editors under consideration

### Eukaryotic Cell Systems

Any of the target sequences of interest, e.g., the target sequences of the gRNAs of the methods for evaluating off-target effects disclosed herein, may be comprised within the genome of a eukaryotic cell, such as a mammalian cell. Accordingly, the target sequence and the third sequence may be comprised within the genome of a mammalian cell. The eukaryotic cell may comprise a murine or human cell, such as an HEK293T cell.

In some embodiments, the cell is a population of cells. Accordingly, in some embodiments, the step of sequencing comprises performing high-throughput sequencing of one or more regions of the genomes of the cells of the population that comprise the target sites and off-target sites that have complementarity to the gRNAs used in the systems.

In some aspects, eukaryotic cell systems for measuring off-target effects (e.g., off-target editing frequencies) of a base editor are provided. These systems may be used in accordance with the disclosed methods. Accordingly, provided herein are systems for determining the off-target editing frequency of a base editor comprising one or more eukaryotic cells each comprising i) a first nucleic acid molecule encoding a base editor comprising a napDNAbp domain; (ii) a second nucleic acid molecule encoding a first guide RNA that is engineered to bind to the napDNAbp domain of the base editor, wherein the first guide RNA comprises a first sequence of at least 10 contiguous nucleotides that is complementary to a target sequence; (iii) a third nucleic acid molecule encoding a nuclease inactive napDNAbp protein; and (iv) a fourth nucleic acid molecule encoding a second gRNA that is engineered to bind to the nuclease inactive napDNAbp protein, wherein the second guide RNA comprises a second sequence of at least 10 contiguous nucleotides that is complementary to a third sequence, whereby the first complex and second complex generate two or more R-loops, and wherein the third sequence has about 60% or less sequence identity to the target sequence.

The disclosed systems may further comprise a third, fourth, fifth, and/or sixth complex, wherein each of the third, fourth, fifth, and/or sixth complexes comprises (v) a second nuclease inactive napDNAbp protein, and (vi) a third guide RNA that is engineered to bind to the second nuclease inactive napDNAbp protein, wherein the third guide RNA comprises a fourth sequence of at least 10 contiguous nucleotides that is complementary to the third sequence. These complexes may be identical or essentially identical to each other, in that they are associated with identical or nearly identical gRNAs that have complementarity to the same off-target sequence. Any one of these complexes may be distinct or essentially identical to the second complex. The second and third guide RNA may share at least 95%, 98%, 98.5%, or 100% sequence identity, e.g., in the backbone of the guide RNA sequence. In certain embodiments, the second and third guide RNA share 100% identity or are the same. Likewise, the first nuclease inactive napDNAbp protein and the second nuclease inactive napDNAbp may be the same.

In some embodiments, any of the nuclease inactive napDNAbp proteins of the described systems may be a dead Cas9 (dCas9) protein. Accordingly, in some embodiments, the second complex comprises a first dCas9 protein, and the third and subsequent complexes comprise a second dCas9 protein. In some embodiments, the nuclease inactive napDNAbp protein of any of the described complexes is a dead Cas9 protein from S. aureus. See FIG. 2B. In some embodiments, the nuclease inactive napDNAbp protein is a dead Cas9 protein from S. pyogenes.

In some embodiments, the eukaryotic cells of the disclosed systems comprise mammalian cells. The eukaryotic cells may comprise human cells, e.g. HEK293T cells.

In some embodiments of these methods, transformed eukaryotic cells are sequenced to validate that mutations arise from C:G to T:A conversions. This sequencing step may be achieved by Sanger sequencing, high-throughput sequencing, whole genome sequencing, and/or other sequencing methods known in the art.

The on-target and Cas9-independent off-target editing rates of various base editors, such as CBEs and ABEs, may be compared by transforming any one of the disclosed eukaryotic cell systems with plasmids encoding these base editors in parallel, and evaluating the deamination rates at on-target and off-target sites for each base editor.

### Prokaryotic Cell Systems and Methods

In other aspects, provided herein are methods (or assays) and systems for evaluating the off-target effects of a base editor in a prokaryotic cell. In various embodiments, these systems are designed for determining the napDNAbp domain-independent (e.g. Cas9-independent) (or gRNA-independent) off-target editing effects of any one of the disclosed base editors.

Accordingly, provided herein are systems comprising one or more prokaryotic cells comprising (i) a nucleic acid molecule that contains a target sequence within a first inactive antibiotic resistance gene, wherein the target sequence within the first inactive antibiotic resistance gene contains a first mutant nucleotide base that yields an active antibiotic resistance gene conferring resistance to a first antibiotic when the first mutant nucleotide base is mutated to a different nucleotide base; (ii) a second nucleic acid molecule that contains a non-target sequence within a second inactive antibiotic resistance gene, wherein the non-target sequence within the second inactive antibiotic resistance gene contains a second mutant nucleotide base that yields an active antibiotic resistance gene conferring resistance to a second antibiotic when the second mutant nucleotide base is mutated to a different base; and (iii) a third nucleic acid molecule encoding a base editor and a guide RNA comprising a sequence of at least 10 contiguous nucleotides that is complementary to the target sequence within the first inactive antibiotic resistance gene. See FIG. 1A. These systems are designed to correct the mutations in the first inactive antibiotic resistance gene and/or the mutation in the second inactive antibiotic resistance gene and compare the frequencies of the mutation correction at each gene.

The non-target sequence of the disclosed bacterial cell systems may comprise a protospacer sequence that has about 70% or less sequence identity to the protospacer of the target sequence. The non-target sequence may comprise a protospacer sequence that has about 60% or less sequence identity to the target sequence. In other embodiments, the non-target sequence may comprise a protospacer sequence that has about 55% or less, 50% or less, 45% or less, 40% or less, 35% or less, or 30% or less sequence identity to the target sequence. The non-target sequence may comprise a protospacer sequence that differs from the protospacer of the target sequence in 5, 6, 7, 8, 9, 10, 11, 12, 13, 14, 15, or greater than 15 nucleotide positions (or has 3, 4, 5, 6, 7, 8, 9, 10, 11, 12, 13, 14, 15 or greater than 15 “mismatches” relative to the target sequence). In certain embodiments, the protospacer of the non-target sequence differs from the protospacer of the target sequence in 6 or more nucleotide positions.

The base editor of the disclosed systems may comprise a cytosine base editor (CBE). The base editor may comprise a Cas9 nickase domain. In certain embodiments, the base editor comprises an SpCas9 nickase (nSpCas9 or SpCas9n) domain. In other embodiments, the base editor comprises a nuclease inactive napDNAbp domain, such as a dCas9 domain. In certain embodiments, the base editor comprises a dSpCas9 domain.

In other embodiments, the base editor under evaluation in the disclosed prokaryotic systems may comprise an adenine base editor (ABE). In other embodiments, the base editor may comprise a transversion base editor, such as a C-to-G base editor (or “CGBE”), a G-to-T base editor (or “GTBE”), an A-to-T base editor (or “ATBE”), or an A-to-C base editor (or “ACBE”).

In some embodiments, the first nucleic acid molecule of (i) is comprised within a heterologous nucleic acid vector, such as a plasmid. In some embodiments, the second nucleic acid molecule of (ii) is comprised in the genome of the one or more bacterial cells.

In some embodiments, the first mutant nucleotide is a cytosine, such that mutating this cytosine to a thymine yields an active antibiotic resistance gene (e.g., chloramphenicol acetyltransferase) conferring resistance to the first antibiotic. The second mutant nucleotide may likewise be a cytosine, such that mutating this cytosine to a thymine yields an active antibiotic resistance gene (e.g., rpoB) conferring resistance to the second antibiotic, for instance yielding an active rpoB gene in the genome of the prokaryotic cell.

In some embodiments, the first antibiotic is chloramphenicol, tetracycline, carbenicillin, chloramphenicol, spectinomycin, or rifampin. In particular embodiments, the first antibiotic is chloramphenicol. In such embodiments, the first inactive antibiotic resistance gene may be the chloramphenicol acetyltransferase gene, or CAT, also known as Cm^{r}. In some embodiments, the second antibiotic is chloramphenicol, tetracycline, carbenicillin, chloramphenicol, spectinomycin, or rifampin. In particular embodiments, the second antibiotic is rifampin. In such embodiments, the second inactive antibiotic resistance gene may be the rpoB gene. CBE-catalyzed C:G-to-T:A mutations in the rpoB gene within E. coli genomes render these bacteria resistant to rifampin.

In some aspects, methods for measuring the off-target effects of a base editor for use in accordance with the disclosed prokaryotic (e.g., bacterial) cell systems are provided. Accordingly, provided herein are methods of determining off-target editing frequency of a base editor comprising: contacting a prokaryotic cell that comprises the second nucleic acid molecule, with (i) the first nucleic acid molecule and (ii) the third nucleic acid molecule; and further contacting the prokaryotic cell with a growth medium comprising the second antibiotic and/or the first antibiotic.

In some embodiments, the first antibiotic is chloramphenicol, tetracycline, carbenicillin, chloramphenicol, spectinomycin, or rifampin. In particular embodiments, the first antibiotic is chloramphenicol. In such embodiments, the first antibiotic resistance gene is chloramphenicol acetyltransferase, or CAT. In some embodiments, the second antibiotic is chloramphenicol, tetracycline, carbenicillin, chloramphenicol, spectinomycin, or rifampin. In particular embodiments, the second antibiotic is rifampin. In such embodiments, the second antibiotic resistance gene may be the rpoB gene.

In an exemplary embodiment of the disclosed methods for measuring the off-target effects in accordance with the disclosed prokaryotic cell systems, prokaryotic cells are transformed with i) a first plasmid encoding a cytosine base editor, and ii) a second plasmid encoding a (non-functional) Cm^{r} gene with an inactivating T:A-to-C:G point mutation, together with a guide RNA with complementarity to Cm^{r}, such that it can direct the CBE to correct this mutation. (No gRNAs with complementarity to the rpoB gene is provided.) Transformed cells are then plated on rifampin and chloramphenicol media. The prokaryotic cells comprise the rpoB gene within their genomes, and the frequency of deamination of the C:G to T:A in the rpoB gene, which confers rifampin resistance, reflects the magnitude of the Cas9-independent off-target frequency. Meanwhile, the magnitude of chloramphenicol resistance inactivation correction by CBE deamination reflects the on-target editing efficiency of the CBE. Accordingly, in the disclosed prokaryotic cell systems and methods, survival rates of colonies on chloramphenicol medium reflect on-target editing efficiency, and survival rates on rifampin medium reflect Cas9-independent deamination activity.

In some embodiments of these methods, the first and/or second antibiotic genes of the surviving prokaryotic colonies are sequenced to validate that mutations arise from C:G to T:A conversions.

The on-target and Cas9-independent off-target editing rates of various base editors, such as CBEs and ABEs, may be compared by transforming any one of the disclosed prokaryotic cell systems with plasmids encoding these base editors in parallel, and evaluating the survival rates of each colony on media comprising the first inactive antibiotic and second inactive antibiotic.

In other aspects of the prokaryotic cell systems, provided herein are systems comprising one or more prokaryotic cells comprising (i) a nucleic acid molecule that contains a target sequence within a gene encoding herpes simplex virus thymidine kinase (HSV-TK), wherein the target sequence contains a nucleotide base that inactivates the HSV-TK gene, thus conferring resistance to a growth medium, when the nucleotide base is mutated to a different nucleotide base; and (ii) a second nucleic acid molecule encoding a base editor (e.g., a CBE) and a guide RNA comprising a sequence of at least 10 contiguous nucleotides that is complementary to the target sequence. The HSV-TK kinase leads to toxicity in the presence of the nucleoside analog deoxyribofuranosyl)-3,4-dihydro-8H-pyrimido-[4,5-c][1,2]oxazin-7-one (“dP”) (34). Accordingly, in some embodiments, the growth medium of the disclosed systems may comprise dP. Off-target C:G-to-T:A mutations in the HSV-TK gene that inactivate the enzyme may lead to survival on medium containing dP.

Further provided are methods for evaluating off-target effects in accordance with the disclosed methods comprising transforming one or more prokaryotic cells with a plasmid comprising a gene encoding HSV-TK and a plasmid encoding a base editor (e.g., a CBE) and guide RNA; and contacting the transformed prokaryotic cells with medium containing 6-(β-D-2-Deoxyribofuranosyl)-3,4-dihydro-8H-pyrimido-[4,5-c][1,2]oxazin-7-one (dP). This medium may further comprise one or more antibiotics, such as carbenicillin and/or spectinomycin.

### Novel Cytosine Base Editors

In various embodiments, the present disclosure provides novel cytosine base editors (CBEs) comprising a napDNAbp domain and a cytidine deaminase domain that enzymatically deaminates a cytosine nucleobase of a C:G nucleobase pair to a uracil. The uracil may be subsequently converted to a thymine (T) by the cell's DNA repair and replication machinery. The mismatched guanine (G) on the opposite strand may subsequently be converted to an adenine (A) by the cell's DNA repair and replication machinery. In this manner, a target C:G nucleobase pair is ultimately converted to a T:A nucleobase pair.

The disclosed novel cytosine base editors exhibit increased on-target editing scope while maintaining minimized off-target DNA editing relative to existing CBEs. The CBEs described herein provide ˜10- to ˜100-fold lower average Cas9-independent off-target DNA editing, while maintaining efficient on-target editing at most positions targetable by existing CBEs. The disclosed novel CBEs comprise novel combinations of mutant cytidine deaminases, such as the YE1, YE2, YEE, and R33A deaminases, and Cas9 domains, and/or novel combinations of mutant cytidine deaminases, Cas9 domains, uracil glycosylase inhibitor (UGI) domains and nuclear localizations sequence (NLS) domains, relative to existing base editors. Existing base editors include BE3, which comprises the structure NH₂-[NLS]-[rAPOBEC1 deaminase]-[Cas9 nickase (D10A)]-[UGI domain]-[NLS]-COOH; BE4, which comprises the structure NH₂-[NLS]-[rAPOBEC1 deaminase]-[Cas9 nickase (D10A)]-[UGI domain]-[UGI domain]-[NLS]-COOH; and BE4max, which is a version of BE4 for which the codons of the base editor-encoding construct has been codon-optimized for expression in human cells.

Zuo et al. recently reported that, when overexpressed in mouse embryos and rice, BE3, the original CBE, induces an average random C:G-to-T:A mutation frequency of 5×10⁻⁸ per bp and 1.7×10⁻⁷ per bp, respectively. See “Cytosine base editor generates substantial off-target single-nucleotide variants in mouse embryos.” Science 364, 289-292 (2019), herein incorporated by reference. Editing was observed in sequences that had little to no similarity to the target sequences. These off-target edits may have arisen from the intrinsic DNA affinity of BE3's deaminase domain, independent of the guide RNA-programmed DNA binding of Cas9. See also Jin et al., Cytosine, but not adenine, base editors induce genome-wide off-target mutations in rice. Science 364, (2019), herein incorporated by reference.

Zuo et al. also found that Cas9-independent off-target editing events were enriched in transcribed regions of the genome, particularly in highly-expressed genes. Some of these were tumor suppressor genes. Accordingly, there is a need in the art to develop base editors that possess low off-target editing frequencies that may avoid undesired activation or inactivation of genes associated with diseases or disorders, such as cancer, and assays that rapidly measure the off-target editing frequencies of these base editors.

Exemplary CBEs may provide an off-target editing frequency of less than 2.0% after being contacted with a nucleic acid molecule comprising a target sequence, e.g., a target nucleobase pair. Further exemplary CBEs provide an off-target editing frequency of less than 1.5% after being contacted with a nucleic acid molecule comprising a target sequence comprising a target nucleobase pair. Further exemplary CBEs may provide an off-target editing frequency of less than 1.25%, less than 1.1%, less than 1%, less than 0.75%, less than 0.5%, less than 0.4%, less than 0.25%, less than 0.2%, less than 0.15%, less than 0.1%, less than 0.05%, or less than 0.025%, after being contacted with a nucleic acid molecule comprising a target sequence.

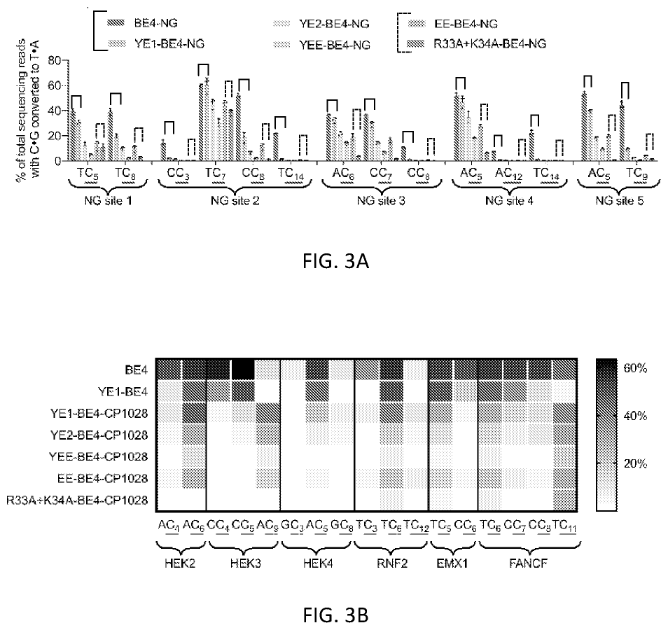

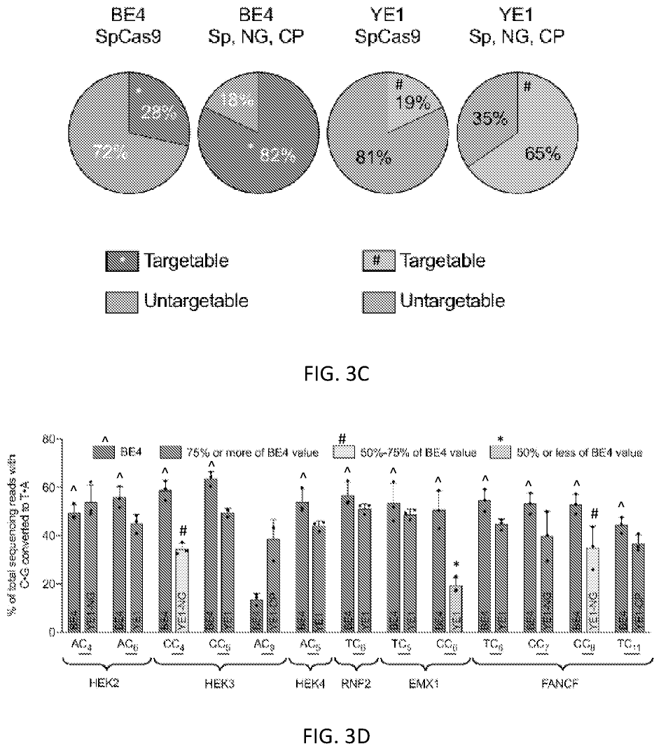

For instance, the novel cytosine base editors YE1-BE4, YE1-CP1028, YE1-SpCas9-NG (also referred to herein as YE1-NG), R33A-BE4, and R33A+K34A-BE4-CP1028, which are described below, may exhibit off-target editing frequencies of less than 0.75% (e.g., about 0.4% or less) while maintaining on-target editing efficiencies of about 60% or more, in target sequences in mammalian cells. Each of these base editors comprises modified cytidine deaminases (e.g., YE1, R33A, or R33A+K34A) and may further comprise a modified napDNAbp domain such as a circularly permuted Cas9 domain (e.g., CP1028) or a Cas9 domain with an expanded PAM window (e.g., SpCas9-NG). These five base editors may be the most preferred for applications in which off-target editing, and in particular Cas9-independent off-target editing, must be minimized. In particular, base editors comprising a YE1 deaminase domain provide efficient on-target editing with greatly decreased Cas9-independent editing, as confirmed by whole-genome sequencing (see FIGS. 3A and 3B).

Exemplary CBEs may further possess an on-target editing efficiency of more than 50% after being contacted with a nucleic acid molecule comprising a target sequence. Further exemplary CBEs possess an on-target editing efficiency of more than 60% after being contacted with a nucleic acid molecule comprising a target sequence. Further exemplary CBEs possess an on-target editing efficiency of more than 65%, more than 70%, more than 75%, more than 80%, more than 82.5%, or more than 85% after being contacted with a nucleic acid molecule comprising a target sequence.

The disclosed CBEs may exhibit indel frequencies of less than 0.75%, less than 0.6%, less than 0.5%, less than 0.4%, less than 0.3%, or less than 0.2% after being contacted with a nucleic acid molecule containing a target sequence. The disclosed CBEs may further exhibit reduced RNA off-target editing relative to existing CBEs. The disclosed CBEs may further result in increased product purity after being contacted with a nucleic acid molecule containing a target sequence relative to existing CBEs.

The disclosed CBEs may further comprise one or more nuclear localization signals (NLSs) and/or two or more uracil glycosylase inhibitor (UGI) domains. Thus, the base editors may comprise the structure: NH₂-[first nuclear localization sequence]-[cytidine deaminase domain]-[napDNAbp domain]-[first UGI domain]-[second UGI domain]-[second nuclear localization sequence]-COOH, wherein each instance of “]-[” indicates the presence of an optional linker sequence. Exemplary CBEs may have a structure that comprises the “BE4max” architecture, with an NH₂-[NLS]-[cytidine deaminase]-[Cas9 nickase]-[UGI domain]-[UGI domain]-[NLS]-COOH structure, having optimized nuclear localization signals and wherein the napDNAbp domain comprises a Cas9 nickase. See FIG. 2A. This BE4max structure was reported to have optimized codon usage for expression in human cells, as reported in Koblan et al., Nat Biotechnol. 2018; 36(9):843-846, herein incorporated by reference.

In other embodiments, exemplary CBEs may have a structure that comprises a modified BE4max architecture that contains a napDNAbp domain comprising a Cas9 variant other than Cas9 nickase, such as SpCas9-NG, xCas9, or circular permutant CP1028. Accordingly, exemplary CBEs may comprise the structure: NH₂-[NLS]-[cytidine deaminase]-[CP1028]-[UGI domain]-[UGI domain]-[NLS]-COOH; NH₂-[NLS]-[cytidine deaminase]-[xCas9]-[UGI domain]-[UGI domain]-[NLS]-COOH; or NH₂-[NLS]-[cytidine deaminase]-[SpCas9-NG]-[UGI domain]-[UGI domain]-[NLS]-COOH, wherein each instance of “]-[” indicates the presence of an optional linker sequence.

In some embodiments, the napDNAbp domain comprises an amino acid sequence that has at least 80%, 85%, 90%, 92.5%, 95%, 98%, or 99.5% sequence identity with SEQ ID NO: 226. In some embodiments, the napDNAbp domain comprises the amino acid sequence of SEQ ID NO: 226. In some embodiments, the napDNAbp domain comprises an amino acid sequence that has at least 80%, 85%, 90%, 92.5%, 95%, 98%, or 99.5% sequence identity with SEQ ID NO: 235. In some embodiments, the napDNAbp domain comprises the amino acid sequence of SEQ ID NO: 235. In some embodiments, the napDNAbp domain comprises an amino acid sequence that has at least 80%, 85%, 90%, 92.5%, 95%, 98%, or 99.5% sequence identity with any one of SEQ ID NOs: 236 or 237. In some embodiments, the napDNAbp domain comprises the amino acid sequence of SEQ ID NO: 236 or 237.

In some embodiments, the UGI domain of any one of the disclosed base editors comprises an amino acid sequence having at least 95% sequence identity to SEQ ID NO: 292. In some embodiments, the UGI domain of any one of the disclosed base editors comprises the amino acid sequence of SEQ ID NO: 292.

The disclosed CBEs may comprise modified (or evolved) cytidine deaminase domains, such as deaminase domains that recognize an expanded PAM sequence, have improved efficiency of deaminating 5′-GC targets, and/or make edits in a narrower target window, In some embodiments, the disclosed cytosine base editors comprise evolved nucleic acid programmable DNA binding proteins (napDNAbp), such as an evolved Cas9.

Exemplary cytosine base editors comprise sequences that are at least 85%, at least 90%, at least 95%, at least 98%, at least 99%, or at least 99.5% identical to the following amino acid sequences, SEQ ID NOs: 257-282.

The cytidine deaminase domains of the disclosed cytosine base editors may comprise variants of wild-type cytidine deaminases. These variants may comprise an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type deaminase. In some embodiments, any of the cytidine deaminase domains may comprise an amino acid sequence having 1, 2, 3, 4, 5, 6, 7, 8, 9, 10, 11, 12, 13, 14, 15, 16, 17, 18, 19, 20, 25, 30, or more than 30 amino acids that differ relative to the amino acid sequence of the wild type enzyme. These differences may comprise nucleotides that have been inserted, deleted, or substituted relative to the amino acid sequence of the wild type enzyme. In some embodiments, the disclosed cytidine deaminase domains contain stretches of about 50, about 75, about 100, about 125, about 150, about 175, about 200, about 300, about 400, about 500, or more than 500 consecutive amino acids in common with the wild type enzyme. In some embodiments, the cytidine deaminase domains comprise truncations at the N-terminus or C-terminus relative to the wild-type enzyme.

Where indicated, “—BE4” refers to the BE4max architecture, or NH₂-[first nuclear localization sequence]-[cytidine deaminase domain]-[32aa linker]-[SpCas9 nickase (nCas9, or nSpCas9) domain]-[9aa linker]-[first UGI domain]-[9aa-linker]-[second UGI domain]-[second nuclear localization sequence]-COOH. Where indicated, “BE4max, modified with SpCas9-NG” and “—SpCas9-NG” refer to a modified BE4max architecture in which the SpCas9 nickase domain has been replaced with an SpCas9-NG, i.e., NH₂-[first nuclear localization sequence]-[cytidine deaminase domain]-[32aa linker]-[SpCas9-NG]-[9aa linker]-[first UGI domain]-[9aa-linker]-[second UGI domain]-[second nuclear localization sequence]-COOH. And where indicated, “BE4-CP1028” refers to a modified BE4max architecture in which the Cas9 nickase domain has been replaced with a S. pyogenes CP1028, i.e., NH₂-[first nuclear localization sequence]-[cytidine deaminase domain]-[32aa linker]-[CP1028]-[9aa linker]-[first UGI domain]-[9aa-linker]-[second UGI domain]-[second nuclear localization sequence]-COOH.

As discussed above, preferred base editors comprise modified cytidine deaminases (e.g., YE1, R33A, or R33A+K34A) and may further comprise a modified napDNAbp domain such as a circularly permuted Cas9 domain (e.g., CP1028) or a Cas9 domain with an expanded PAM window (e.g., SpCas9-NG). The napDNAbp domains in the following amino acid sequences are indicated in italics.

BE4max
(SEQ ID NO: 257)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKSKKLK
SVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
YE1-BE4
(SEQ ID NO: 258)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSYSPCGECSRAITEFLSRYP
HVTLFIYIARLYHHADPENRQGLRDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKSKKLK
SVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
YE2-BE4
(SEQ ID NO: 259)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSYSPCGECSRAITEFLSRYP
HVTLFIYIARLYHHADPRNRQGLEDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKSKKLK
SVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
YEE-BE4
(SEQ ID NO: 260)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSYSPCGECSRAITEFLSRYP
HVTLFIYIARLYHHADPENRQGLEDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSEEPGESESAEPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPESASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQEQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKSKKLK
SVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
EE-BE4
(SEQ ID NO: 261)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSWSPCGECSRAITEFLSRYP
HVTLFIYIARLYHHADPENRQGLEDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSEYPGYSESAYPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQEQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKSKKLK
SVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
R33A-BE4
(SEQ ID NO: 262)
MKRTADGSEFESPKKKRKVSSETGPVAVDPTLRRRIEPHEFEVFFDPRELAKETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSEYPGYSESAYPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQEQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKSKKLK
SVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
R33A + K34A-BE4
(SEQ ID NO: 263)
MKRTADGSEFESPKKKRKVSSETGPVAVDPTLRRRIEPHEFEVFFDPRELAAETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSEYPGYSESAYPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKSKKLK
SVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
APOBEC3A (A3A)-BE4
(SEQ ID NO: 264)
MKRTADGSEFESPKKKRKVSEASPASGPRHLMDPHIFTSNFNNGIGRHKTYLCYEVERLDNG
TSVKMDQHRGFLHNQAKNLLCGFYGRHAELRFLDLVPSLQLDPAQIYRVTWFISWSPCFSW
GCAGEVRAFLQENTHVRLRIFAARIYDYDPLYKEALQMLRDAGAQVSIMTYDEFKHCWDT
FVDHQGCPFQPWDGLDEHSQALSGRLRAILQNQGNSGGSSGGSSGSETPGTSESATPESSGG
SSGGSDKKYSIGEAIGTNSVGWAVITDEYKVPSKKFKVEGNTDRHSIKKNEIGALLFDSGETAEATR
LKRTARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAEDA
KLQLSKDTYDDDLDNLLAQIGDQYADLFLAAKNLSDAILLSDILRVNTEITKAPLSASMIKRYDEHH
QDLTLLKALVRQQLPEKYKEIFFDQSKNGYAGYIDGGASQEEFYKFIKPILEKMDGTEELLVKLNR
EDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKGQKNSRERMKRIEEGIK
ELGSQILKEHPVENTQLQNEKLYLYYLQNGRDMYVDQELDINRLSDYDVDHIVPQSFLKDDSIDN
KVLTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKESILPKRNSD
KLIARKKDWDPKKYGGFDSPTVAYSVLVVAKVEKGKSKKLKSVKELLGITIMERSSFEKNPIDFLE
AKGYKEVKKDLIIKLPKYSLFELENGRKRMLASAGELQKGNELALPSKYVNFLYLASHYEKLKGSP
EDNEQKQLFVEQHKHYLDEIIEQISEFSKRVILADANLDKVLSAYNKHRDKPIREQAENIIHLFTLT
NLGAPAAFKYFDTTIDRKRYTSTKEVLDATLIHQSITGLYETRIDLSQLGGDSGGSGGSGGSYNLS
DIIEKETGKQLVIQESILMLPEEVEEVIGNKPESDILVHTAYDESTDENVMLLTSDAPEYKPWA
LVIQDSNGENKIKMLSGGSGGSGGSTNLSDIIEKETGKQLVIQESILMLPEEVEEVIGNKPESD
ILVHTAYDESTDENVMLLTSDAPEYKPWALVIQDSNGENKIKMLSGGSKRTADGSEFEPKKK
RKV
APOBEC3B (A3B)-BE4
(SEQ ID NO: 265)
MKRTADGSEFESPKKKRKVNPQIRNPMERMYRDTFYDNFENEPILYGRSYTWLCYEVKIKR
GRSNLLWDTGVFRGQVYFKPQYHAEMCFLSWFCGNQLPAYKCFQITWFVSWTPCPDCVAK
LAEFLSEHPNVTLTISAARLYYYWERDYRRALCRLSQAGARVTIMDYEEFAYCWENFVYNE
GQQFMPWYKFDENYAFLHRTLKEILRYLMDPDTFTFNFNNDPLVLRRRQTYLCYEVERLDN
GTWVLMDQHMGFLCNEAKNLLCGFYGRHAELRFLDLVPSLQLDPAQIYRVTWFISWSPCFS
WGCAGEVRAFLQENTHVRLRIFAARIYDYDPLYKEALQMLRDAGAQVSIMTYDEFEYCWD
TFVYRQGCPFQPWDGLEEHSQALSGRLRAILQNQGNSGGSSGGSSGSETPGTSESATPESSG
GSSGGSDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTYNQ
LFEENPINASGVDAKAILSARLSKSRRLENLIAQLPGEKKNGLFGNLIALSLGLTPNFKSNFLEAED
AKLQLSKDTYDDDLDNLLAQIGDQYADLFLAAKNLSDAILLSDIERVNTEITKAPESASMIKRYDEH
HQDLTLLKALVRQQLPEKYKEIFFDQSKNGYAGYIDGGASQEEFYKFIKPILEKMDGTEELLVKL
NREDLLRKQRTFDNGSIPHQIHLGELHAILRRQEDFYPFLKDNREKIEKILTFRIPYYVGPLARGNS
RFAWMTRKSEETITPWNFEEWDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQVSGQGDSLHE
HIANLAGSPAIKKGILQTVKVVDELVKVMGRHKPENIVIEMARENQTTQKGQKNSRERMKRIEEGI
KELGSQILKEHPVENTQLQNEKLYLYYLQNGRDMYVDQELDINRLSDYDVDHIVPQSFLKDDSID
NKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGSGGSGGSYNL
SDIIEKETGKQLVIQESILMLPEEVEEVIGNKPESDILVHTAYDESTDENVMLLTSDAPEYKPW
ALVIQDSNGENKIKMLSGGSGGSGGSTNLSDIIEKETGKQLVIQESILMLPEEVEEVIGNKPES
DILVHTAYDESTDENVMLLTSDAPEYKPWALVIQDSNGENKIKMLSGGSKRTADGSEFEPKK
KRKV
APOBEC3G (A3G)-BE4
(SEQ ID NO: 266)
MKRTADGSEFESPKKKRKVKPHFRNTVERMYRDTFSYNFYNRPILSRRNTVWLCYEVKTK
GPSRPPLDAKIFRGQVYSELKYHPEMRFFHWFSKWRKLHRDQEYEVTWYISWSPCTKCTR
DMATFLAEDPKVTLTIFVARLYYFWDPDYQEALRSLCQKRDGPRATMKIMNYDEFQHCWS
KFVYSQRELFEPWNNLPKYYILLHIMLGEILRHSMDPPTFTFNFNNEPWVRGRHETYLCYEV
ERMHNDTWVLLNQRRGFLCNQAPHKHGFLEGRHAELCFLDVIPFWKLDLDQDYRVTCFTS
WSPCFSCAQEMAKFISKNKHVSLCIFTARIYDDQGRCQEGLRTLAEAGAKISIMTYSEFKHC
WDTFVDHQGCPFQPWDGLDEHSQDLSGRLRAILQNQENSGGSSGGSSGSETPGTSESATPES
SGGSSGGSDKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLNPDNSDVDKLFIQLVQT
YNQLFEENPINASGVDAKAILSARLSKSRRLENLIAQLPGEKKNGLFGNLIALSLGLTPNFKSNFDL
AEDAKLQLSKDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFEKDNREKIEKILTFRIPYYVGPLAR
GNSRFAWMTRKSEETITPWNFEEWDKGASAQSFIERMTNFDKNLPNLKVLPKHSLLYEYFTVYN
ELTKVKYVTEGMRKPAFLSGEQKKAIVDLLFKTNRKVTVKQLKEDYFKKIECFDSVEISGVEDRF
NASLGTYHDLLKIIKDKDFLDNEENEDILEDIVLTLTLFEDREMIEERLKTYAHLFDDKVMKQLKR
RRYTGWGRLSRKLINGIRDKQSGKTILDFLKSDGFANRNFMQLIHDDSLTFKEDIQKAQVSGQGD
SLHEHIANLAGSPAIKKGILQTVKVVDELVKVMGRHKPENIVIEMARENQTTQKGQKNSRERMKR
IEEGIKELGSQILKEHPVENTQLQNEKLYLYYLQNGRDMYVDQELDINRLSDYDVDHIVPQSFLK
DDSIDNKVLTRSDKNRGKSDNVPSEEVVKKMKNYWRQLLNAKLITQRKFDNLTKAERGGESELD
KAGFIKRQLVETRQITKHVAQILDSRMNTKYDENDKLIREVKVITLKSKLVSDFRKDFQFYKVREIN
NYHHAHDAYLNAVVGTALIKKYPKLESEFVYGDYKVYDVRKMIAKSEQEIGKATAKYFFYSNIMNF
FKTEITLANGEIRKRPLIETNGETGEIVWDKGRDFATVRKVESMPQVNIVKKTEVQTGGFSKESILP
KRNSDKLIARKKDWDPKKYGGFDSPTVAYSVLVVAKVEKGKSKKEKSVKELLGITIMERSSFEKNP
IDFLEAKGYKEVKKDLIIKLPKYSLFELENGRKRMLASAGELQKGNELALPSKYVNFLYLASHYEK
LKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGDSGGSGGSGGS
TNLSDIIEKETGKQLVIQESILMLPEEVEEVIGNKPESDILVHTAYDESTDENVMLLTSDAPEY
KPWALVIQDSNGENKIKMLSGGSGGSGGSTNLSDIIEKETGKQLVIQESILMLPEEVEEVIGN
KPESDILVHTAYDESTDENVMLLTSDAPEYKPWALVIQDSNGENKIKMLSGGSKRTADGSEF
EPKKKRKV
AID-BE4
(SEQ ID NO: 267)
MKRTADGSEFESPKKKRKVDSLLMNRRKFLYQFKNVRWAKGRRETYLCYVVKRRDSATSF
SLDFGYLRNKNGCHVELLFLRYISDWDLDPGRCYRVTWFTSWSPCYDCARHVADFLRGNP
NLSLRIFTARLYFCEDRKAEPEGLRRLHRAGVQIAIMTFKDYFYCWNTFVENHERTFKAWE
GLHENSVRLSRQLRRILLPLYEVDDLRDAFRTLGLSGGSSGGSSGSETPGTSESATPESSGGSS
GGSDKKYSIGLAIGTNSVGWAVITDEYKVPSKKFKVLGNTDRHSIKKNLIGALLFDSGETAEATRLK
RTARRRYTRRKNRICYEQEIFSNEMAKVDDSFFHRLEESFLVEEDKKHERHPIFGNIVDEVAYHEK
YPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQEPGEKKNGLFGNLIALSLGLTPNFKSNFDLAEDAKL
QLSKDTYDDDLDNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPIELKMDGTEELLVKLNRE
DLLRKQRTFDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFDDKVMKQLKRRRYTGW
GRLSRKLINGIRDKQSGKTILDFLKSDGFANRNFMQLIHDDSLTFKEDIQKAQVSGQGDSLHEHI
ANLAGSPAIKKGILQTVKVVDELVKVMGRHKPENIVIEMARENQTTQKGQKNSRERMKRIEEGIK
ELGSQILKEHPVENTQLQNEKLYLYYLQNGRDMYVDQELDINRLSDYDVDHIVPQSFLKDDSIDN
KVLTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKESILPKRNSD
KLIARKKDWDPKKYGGFDSPTVAYSVLVVAKVEKGKSKKLKSVKELLGITIMERSSFEKNPIDFEL
AKGYKEVKKDLIIKLPKYSEFELENGRKRMLASAGELQKGNELALPSKYVNFLYLASHYEKLKGSP
EDNEQKQLFVEQHKHYLDEIIEQISEFSKRVILADANLDKVLSAYNKHRDKPIREQAENIIHLFTLT
NLGAPAAFKYFDTTIDRKRYTSTKEVLDATLIHQSITGLYETRIDLSQLGGDSGGSGGSGGSYNLS
DIIEKETGKQLVIQESILMLPEEVEEVIGNKPESDILVHTAYDESTDENVMLLTSDAPEYKPWA
LVIQDSNGENKIKMLSGGSGGSGGSTNLSDIIEKETGKQLVIQESILMLPEEVEEVIGNKPESD
ILVHTAYDESTDENVMLLTSDAPEYKPWALVIQDSNGENKIKMLSGGSKRTADGSEFEPKKK
RKV
CDA-BE4
(SEQ ID NO: 268)
MKRTADGSEFESPKKKRKVTDAEYVRIHEKLDIYTFKKQFFNNKKSVSHRCYVLFELKRRG
ERRACFWGYAVNKPQSGTERGIHAEIFSIRKVEEYLRDNPGQFTINWYSSWSPCADCAEKIL
EWYNQELRGNGHTLKIWACKLYYEKNARNQIGLWNLRDNGVGLNVMVSEHYQCCRKIFIQ
SSHNQLNENRWLEKTLKRAEKRRSELSIMIQVKILHTTKSPAVSGGSSGGSSGSETPGTSESA
TPESSGGSSGGSDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIAESLGLTPNFKSN
FDLAEDAKLQLSKDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFEKDNRLKIEKILTFRIPYYVGP
LARGNSRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVPQSFL
KDDSIDNKVLTRSDKNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSKLVSDFRKDFQFYKVRE
INNYHHAHDAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQTGGFSKESI
LPKRNSDKLIARKKDWDPKKYGGFDSPTVAYSVLVVAKVEKGKSKKLKSVKELLGITIMERSSFEK
NPIDLEEAKGYKEVKKDLIIKEPKYSEFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAEN
IIHLFTLTNLGAPAAFKYFDTTIDRKRYTSTKEVLDATLIHQSITGLYETRIDLSQLGGDSGGSGGSG
GSTNLSDIIEKETGKQLVIQESILMLPEEVEEVIGNKPESDILVHTAYDESTDENVMLLTSDAP
EYKPWALVIQDSNGENKIKMLSGGSGGSGGSTNLSDIIEKETGKQLVIQESILMLPEEVEEVIG
NKPESDILVHTAYDESTDENVMLLTSDAPEYKPWALVIQDSNGENKIKMLSGGSKRTADGSE
FEPKKKRKV
FERNY-BE4
(SEQ ID NO: 269)
MKRTADGSEFESPKKKRKVFERNYDPRELRKETYLLYEIKWGKSGKLWRHWCQNNRTQHA
EVYFLENIFNARRFNPSTHCSITWYLSWSPCAECSQKIVDFLKEHPNVNLEIYVARLYYHEDE
RNRQGLRDLVNSGVTIRIMDLPDYNYCWKTFVSDQGGDEDYWPGHFAPWIKQYSLKLSGG
SSGGSSGSEYPGYSESAYPESSGGSSGGSDKKYSIGLAIGTNSVGWAVITDEYKVPSKKFKVLGNT
DRHSIKKNLIGALLFDSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALAHMIKFRGHFLIE
GDLNPDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDAILLS
DILRVNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPFLKD
NREKIEKILTFRIPYYVGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQE
LDINRLSDYDVDHIVPQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIREVKVITL
KSKLVSDFRKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFATVRKVLSMP
QVNIVKKTEVQTGGFSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFELENGRKRMLASAGELQK
GNELALPSKYVNFLYLASHYEKLKGSPEDNEQKQLFVEQHKHYLDEIIEQISEFSKRVILADANLD
KVLSAYNKHRDKPIREQAENIIHLFTLTNLGAPAAFKYFDTTIDRKRYTSTKEVLDATLIHQSITGLY
ETRIDLSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDIIEKETG
KQLVIQESILMLPEEVEEVIGNKPESDILVHTAYDESTDENVMLLTSDAPEYKPWALVIQDSN
GENKIKMLSGGSKRTADGSEFEPKKKRKV
evolved APOBEC3A (eA3A)-BE4
(SEQ ID NO: 270)
MKRTADGSEFESPKKKRKVEASPASGPRHLMDPHIFTSNFNNGIGRHKTYLCYEVERLDNG
TSVKMDQHRGFLHGQAKNLLCGFYGRHAELRFLDLVPSLQLDPAQIYRVTWFISWSPCFSW
GCAGEVRAFLQENTHVRLRIFAARIYDYDPLYKEALQMLRDAGAQVSIMTYDEFKHCWDT
FVDHQGCPFQPWDGLDEHSQALSGRLRAILQNQGNSGGSSGGSSGSETPGTSESATPESSGG
SSGGSDKKYSIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAEDA
KLQLSKDTYDDDLDNLLAQIGDQYADLFLAAKNLSDAILLSDILRVNTEITKAPLSASMIKRYDEHH
QDLTLLKALVRQQLPEKYKEIFFDQSKNGYAGYIDGGASQEEFYKFIKPILEKMDGTEELLVKLNR
EDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKGQKNSRERMKRIEEGIK
ELGSQILKEHPVENTQLQNEKLYLYYLQNGRDMYVDQELDINRLSDYDVDHIVPQSFLKDDSIDN
KVLTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKESILPKRNSD
KLIARKKDWDPKKYGGFDSPTVAYSVLVVAKVEKGKSKKLKSVKELLGITIMERSSFEKNPIDFLE
AKGYKEVKKDLIIKEPKYSLFELENGRKRMLASAGELQKGNELALPSKYVNFLYLASHYEKLKGSP
EDNEQKQLFVEQHKHYLDEIIEQISEFSKRVILADANLDKVLSAYNKHRDKPIREQAENIIHLFTLT
NLGAPAAFKYFDTTIDRKRYTSTKEVLDATLIHOSITGLYETRIDLSQLGGDSGGSGGSGGSTNLS
DIIEKETGKQLVIQESILMLPEEVEEVIGNKPESDILVHTAYDESTDENVMLLTSDAPEYKPWA
LVIQDSNGENKIKMLSGGSGGSGGSTNLSDIIEKETGKQLVIQESILMLPEEVEEVIGNKPESD
ILVHTAYDESTDENVMLLTSDAPEYKPWALVIQDSNGENKIKMLSGGSKRTADGSEFEPKKK
RKV
AALN-BE4
(SEQ ID NO: 271)
MKRTADGSEFESPKKKRKVSSETGPVAVDPTLRRRIEPHEFEVFFDPRELAAETCLLYEINWG
GRHSIWRHTSQNTNKHVEVNFIEKFTTERYFCPNTRCSITWFLSWSPCGECSRAITEFLSRYP
HVTLFIYIARLYHLANPRNRQGLRDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSEYPGYSESAYPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKSKKLK
SVKELLGITIMERSSFEKNPIDFLEAKGYKEVKKDLIIKLPKYSLFELENGRKRMLASAGELQKGNE
LALPSKYVNFLYLASHYEKLKGSPEDNLQKQLFVEQHKHYLDEIIEQISEFSKRVILADANLDKVLS
AYNKHRDKPIREQAENIIHEFTLTNLGAPAAFKYFDTTIDRKRYTSTKEVLDATLIHQSITGLYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
BE4max, modified with SpCas9-NG
(SEQ ID NO: 272)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSEYPGYSESAYPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESIRPKRNSDKLIARKKDWDPKKYGGFVSPTVAYSVLVVAKVEKGKSKKLK
SVKELLGITIMERSSFEKNPIDFLEAKGYKEVKKDLIIKLPKYSLFELENGRKRMLASARFLQKGNE
LALPSKYVNFLYLASHYEKLKGSPEDNEQKQLFVEQHKHYLDEIIEQISEFSKRVILADANLDKVLS
AYNKHRDKPIREQAENIIHLFTLTNLGAPRAFKYFDTTIDRKVYRSTKEVLDATLIHQSITGLYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
YE1-SpCas9-NG base editor (YE1-NG)
(SEQ ID NO: 273)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSYSPCGECSRAITEFLSRYP
HVTLFIYIARLYHHADPENRQGLRDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESIRPKRNSDKLIARKKDWDPKKYGGFVSPTVAYSVLVVAKVEKGKSKKLK
SVKELLGITIMERSSFEKNPIDFLEAKGYKEVKKDLIIKLPKYSLFELENGRKRMLASARFLQKGNE
LALPSKYVNFLYLASHYEKLKGSPEDNEQKQLFVEQHKHYLDEIIEQISEFSKRVILADANLDKVLS
AYNKHRDKPIREQAENIIHLFTLTNLGAPRAFKYFDTTIDRKVYRSTKEVLDATLIHQSITGLYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
YE2-SpCas9-NG base editor
(SEQ ID NO: 274)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSYSPCGECSRAITEFLSRYP
HVTLFIYIARLYHHADPRNRQGLEDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESIRPKRNSDKLIARKKDWDPKKYGGFVSPTVAYSVLVVAKVEKGKSKKLK
SVKELLGITIMERSSFEKNPIDFLEAKGYKEVKKDLIIKLPKYSLFELENGRKRMLASARFLQKGNE
LALPSKYVNFLYLASHYEKLKGSPEDNEQKQLFVEQHKHYLDEIIEQISEFSKRVILADANLDKVLS
AYNKHRDKPIREQAENIIHLFTLTNLGAPRAFKYFDTTIDRKVYRSTKEVLDATLIHQSITGLYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
YEE-SpCas9-NG base editor
(SEQ ID NO: 275)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSYSPCGECSRAITEFLSRYP
HVTLFIYIARLYHHADPENRQGLEDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSEEPGESESAEPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPLEKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESIRPKRNSDKLIARKKDWDPKKYGGFVSPTVAYSVLVVAKVEKGKSKKLK
SVKELLGITIMERSSFEKNPIDFLEAKGYKEVKKDLIIKEPKYSLFELENGRKRMLASARFLQKGNE
LALPSKYVNFLYLASHYEKLKGSPEDNEQKQLFVEQHKHYLDEIIEQISEFSKRVILADANLDKVLS
AYNKHRDKPIREQAENIIHLFTLTNLGAPRAFKYFDTTIDRKVYRSTKEVLDATEIHQSITGEYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
EE-SpCas9-NG base editor
(SEQ ID NO: 276)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSWSPCGECSRAITEFLSRYP
HVTLFIYIARLYHHADPENRQGLEDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSEYPGYSESAYPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESIRPKRNSDKLIARKKDWDPKKYGGFVSPTVAYSVLVVAKVEKGKSKKLK
SVKELLGITIMERSSFEKNPIDFLEAKGYKEVKKDLIIKEPKYSLFELENGRKRMLASARFLQKGNE
LALPSKYVNFLYLASHYEKLKGSPEDNEQKQLFVEQHKHYLDEIIEQISEFSKRVILADANLDKVLS
AYNKHRDKPIREQAENIIHLFTLTNLGAPRAFKYFDTTIDRKVYRSTKEVLDATEIHQSITGEYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
R33A + K34A-SpCas9-NG base editor
(SEQ ID NO: 277)
MKRTADGSEFESPKKKRKVSSETGPVAVDPTLRRRIEPHEFEVFFDPRELAAETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSEYPGYSESAYPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQV
NIVKKTEVQTGGFSKESIRPKRNSDKLIARKKDWDPKKYGGFVSPTVAYSVLVVAKVEKGKSKKLK
SVKELLGITIMERSSFEKNPIDFLEAKGYKEVKKDLIIKEPKYSLFELENGRKRMLASARFLQKGNE
LALPSKYVNFLYLASHYEKLKGSPEDNEQKQLFVEQHKHYLDEIIEQISEFSKRVILADANLDKVLS
AYNKHRDKPIREQAENIIHLFTLTNLGAPRAFKYFDTTIDRKVYRSTKEVLDATEIHQSITGEYETRI
DLSQLGGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSKRTADGSEFEPKKKRKV
YE1-CP1028 base editor (YE1-BE4-CP1028, or YE1-CP)
(SEQ ID NO: 278)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSYSPCGECSRAITEFLSRYP
HVTLFIYIARLYHHADPENRQGLRDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSETPGTSESATPESSGGSSGGSEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAENIIHLF
TLTNLGAPAAFKYFDTTIDRKRYTSTKEVLDATLIHQSITGLYETRIDLSQLGGDGGSGGSGG
SGGSGGSGGSGGMDKKYSIGLAIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLF
IQLVQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKNLPNEKVLPKHSLLYE
YFTVYNELTKVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTTQKGQKNSR
ERMKRIEEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQSGGSGGSGGSY
NLSDIIEKETGKQLVIQESILMLPEEVEEVIGNKPESDILVHTAYDESTDENVMLLTSDAPEYK
PWALVIQDSNGENKIKMLSGGSGGSGGSTNLSDIIEKETGKQLVIQESILMLPEEVEEVIGNKP
ESDILVHTAYDESTDENVMLLTSDAPEYKPWALVIQDSNGENKIKMLSGGSKRTADGSEFEP
KKKRKV
YE2-CP1028 base editor (YE2-BE4-CP1028)
(SEQ ID NO: 279)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSYSPCGECSRAITEFLSRYP
HVTLFIYIARLYHHADPRNRQGLEDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSETPGTSESATPESSGGSSGGSEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAENIIHLF
TLTNLGAPAAFKYFDTTIDRKRYTSTKEVLDATLIHQSITGLYETRIDLSQLGGDGGSGGSGG
SGGSGGSGGSGGMDKKYSIGLAIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLF
IQLVQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKNLPNEKVLPKHSLLYE
YFTVYNELTKVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTTQKGQKNSR
ERMKRIEEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQSGGSGGSGGST
NLSDIIEKETGKQLVIQESILMLPEEVEEVIGNKPESDILVHTAYDESTDENVMLLTSDAPEYK
PWALVIQDSNGENKIKMLSGGSGGSGGSTNLSDIIEKETGKQLVIQESILMLPEEVEEVIGNKP
ESDILVHTAYDESTDENVMLLTSDAPEYKPWALVIQDSNGENKIKMLSGGSKRTADGSEFEP
KKKRKV
YEE-CP1028 base editor (YEE-BE4-CP1028)
(SEQ ID NO: 280)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSYSPCGECSRAITEFLSRYP
HVTLFIYIARLYHHADPENRQGLEDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSETPGTSESATPESSGGSSGGSEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAENIIHLF
TLTNLGAPAAFKYFDTTIDRKRYTSTKEVLDATLIHQSITGLYETRIDLSQLGGDGGSGGSGG
SGGSGGSGGSGGMDKKYSIGLAIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIF
GNIVDLVAYHEKYPTIYHLRKKLVDSTDKADLRLIYLALAHMIKFRGHFLIEGDLNPDNSDVDKLF
IQLVQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKNLPNEKVLPKHSLLYE
YFTVYNELTKVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTTQKGQKNSR
ERMKRIEEGIKELGSQILKEHPVENTQLQNEKLYLYYLQNGRDMYVDQELDINRESDYDVDHIVP
QSFLKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDFQFY
KVREINNYHHAHDAYLNAWGTALIKKYPKLESEFVYGDYKVYDVRKMIAKSEQSGGSGGSGGSY
NLSDIIEKETGKQLVIQESILMLPEEVEEVIGNKPESDILVHTAYDESTDENVMLLTSDAPEYK
PWALVIQDSNGENKIKMLSGGSGGSGGSTNLSDIIEKETGKQLVIQESILMLPEEVEEVIGNKP
ESDILVHTAYDESTDENVMLLTSDAPEYKPWALVIQDSNGENKIKMLSGGSKRTADGSEFEP
KKKRKV
EE-CP1028 base editor (EE-BE4-CP1028)
(SEQ ID NO: 281)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSWSPCGECSRAITEFLSRYP
HVTLFIYIARLYHHADPENRQGLEDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSETPGTSESATPESSGGSSGGSEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAENIIHLF
TLTNLGAPAAFKYFDTTIDRKRYTSTKEVLDATLIHQSITGLYETRIDLSQLGGDGGSGGSGG
SGGSGGSGGSGGMDKKYSIGLAIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLF
IQLVQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHEGELHAILRRQEDFYPFLKDNREKIEKILTFRIPYY
VGPLARGNSRFAWMTRKSEETITPWNFEEVVDKGASAQSFIERMTNFDKNLPNEKVLPKHSLLYE
YFTVYNELTKVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTTQKGQKNSR
ERMKRIEEGIKELGSQILKEHPVENTQLQNEKEYLYYLQNGRDMYVDQELDINRLSDYDVDHIVP
QSFLKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQSGGSGGSGGSY
NLSDIIEKETGKQLVIQESILMLPEEVEEVIGNKPESDILVHTAYDESTDENVMLLTSDAPEYK
PWALVIQDSNGENKIKMLSGGSGGSGGSTNLSDIIEKETGKQLVIQESILMLPEEVEEVIGNKP
ESDILVHTAYDESTDENVMLLTSDAPEYKPWALVIQDSNGENKIKMLSGGSKRTADGSEFEP
KKKRKV
R33A + K34A-CP1028 base editor (R33A + 34A-BE4-CP1028)
(SEQ ID NO: 282)
MKRTADGSEFESPKKKRKVSSETGPVAVDPTLRRRIEPHEFEVFFDPRELAAETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRY
PHLWVRLYVLELYCIILGLPPCLNILRRKQPQLTFFTIALQSCHYQRLPPHILWATGLKSGGSS
GGSSGSETPGTSESATPESSGGSSGGSEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAENIIHLF
TLTNLGAPAAFKYFDTTIDRKRYTSTKEVLDATLIHQSITGLYETRIDLSQLGGDGGSGGSGG
SGGSGGSGGSGGMDKKYSIGLAIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLF
IQLVQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQEEFYKFIKPIELKMD
GTEELLVKLNREDLLRKQRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKNLPNLEKVLPKHSLLYE
YFTVYNELTKVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTTQKGQKNSR
ERMKRIEEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQSGGSGGSGGSY
NLSDIIEKETGKQLVIQESILMLPEEVEEVIGNKPESDILVHTAYDESTDENVMLLTSDAPEYK
PWALVIQDSNGENKIKMLSGGSGGSGGSTNLSDIIEKETGKQLVIQESILMLPEEVEEVIGNKP
ESDILVHTAYDESTDENVMLLTSDAPEYKPWALVIQDSNGENKIKMLSGGSKRTADGSEFEP
KKKRKV

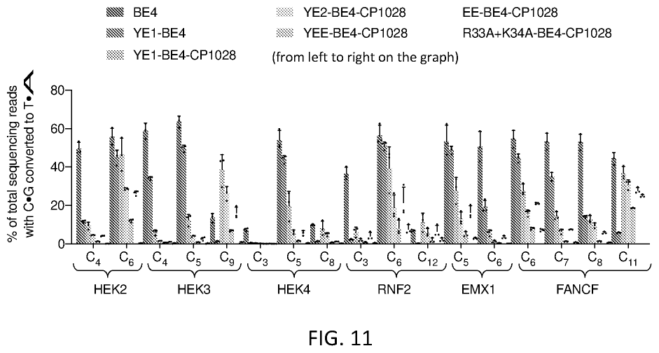

These disclosed CBEs exhibit low off-target editing frequencies, and in particular low Cas9-independent off-target editing frequencies, while exhibiting high on-target editing efficiencies. For example, the YE1-BE4, YE1-CP1028, YE1-SpCas9-NG, R33A-BE4, and R33A+K34A-BE4-CP1028 base editors may exhibit off-target editing frequencies of less than 0.75% (e.g., about 0.4% or less) while maintaining on-target editing efficiencies of about 60% or more, in target sequences in mammalian cells. (See, e.g., FIGS. 11, 15A, 15B and 17.) The Examples of the present disclosure suggest that CBEs with cytidine deaminases that have a low instrinsic catalytic efficiency (k_{cat}/Kₘ) for cytosine-containing ssDNA substrates exhibit reduced Cas9-independent off-target deamination.

### NapDNAbP Domains

cytidine deaminase The base editors described herein comprise a nucleic acid programmable DNA binding (napDNAbp) domain. The napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp domain to localize and bind to a complementary sequence of the target strand. Binding of the napDNAbp domain to a complementary sequence enables the nucleobase modification domain of the base editor to access and enzymatically deaminate a target cytosine base in the target strand.

The napDNAbp can be a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek et al., Science 337:816-821(2012), the entire contents of which is hereby incorporated by reference.

Without wishing to be bound by any particular theory, the binding mechanism of a napDNAbp-guide RNA complex, in general, includes the step of forming an R-loop whereby the napDNAbp induces the unwinding of a double-strand DNA target, thereby separating the strands in the region bound by the napDNAbp. The guideRNA protospacer then hybridizes to the “target strand.” This displaces a “non-target strand” that is complementary to the target strand, which forms the single strand region of the R-loop. In some embodiments, the napDNAbp includes one or more nuclease activities, which cuts the DNA leaving various types of lesions (e.g., a nick in one strand of the DNA). For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand.

The below description of various napDNAbps which can be used in connection with the disclosed nucleobase modification domains is not meant to be limiting in any way. The base editors may comprise the canonical SpCas9, or any ortholog Cas9 protein, or any variant Cas9 protein-including any naturally occurring variant, mutant, or otherwise engineered version of Cas9—that is known or which can be made or evolved through a directed evolution or otherwise mutagenic process. In various embodiments, the napDNAbp has a nickase activity, i.e., only cleave one strand of the target DNA sequence. In other embodiments, the napDNAbp has an inactive nuclease, e.g., are “dead” proteins. Other variant Cas9 proteins that may be used are those having a smaller molecular weight than the canonical SpCas9 (e.g., for easier delivery) or having modified or rearranged primary amino acid sequence (e.g., the circular permutant forms). The base editors described herein may also comprise Cas9 equivalents, including Cas12a/Cpf1 and Cas12b proteins. The napDNAbps used herein (e.g., an SpCas9 or SpCas9 variant) may also contain various modifications that alter/enhance their PAM specifities. The disclosure contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a reference SpCas9 canonical sequence (set forth in SEQ ID NO: 213), a reference SaCas9 canonical sequence (set forth in SEQ ID NO: 216) or a reference Cas9 equivalent (e.g., Cas12a/Cpf1).

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50, 100, 200, 500, or more base pairs from the first or last nucleotide of a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A in reference to the canonical SpCas9 sequence, or to equivalent amino acid positions in other Cas9 variants or Cas9 equivalents.

As used herein, the term “Cas protein” refers to a full-length Cas protein obtained from nature, a recombinant Cas protein having a sequences that differs from a naturally occurring Cas protein, or any fragment of a Cas protein that nevertheless retains all or a significant amount of the requisite basic functions needed for the disclosed methods, i.e., (i) possession of nucleic-acid programmable binding of the Cas protein to a target DNA, and (ii) ability to nick the target DNA sequence on one strand. The Cas proteins contemplated herein embrace CRISPR Cas9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference.

The term “Cas9” or “Cas9 domain” embraces any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular napDNAbp that is employed in the base editors of the disclosure.

Additional Cas9 sequences and structures are well known to those of skill in the art (see, e.g., “Complete genome sequence of an M1 strain of Streptococcus pyogenes.” Ferretti et al., Proc. Natl. Acad. Sci. U.S.A. 98:4658-4663(2001); “CRISPR RNA maturation by trans-encoded small RNA and host factor RNase III.” Deltcheva E., et al., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference), and also provided below.

Examples of Cas9 and Cas9 equivalents are provided as follows; however, these specific examples are not meant to be limiting. The base editors of the present disclosure may use any suitable napDNAbp, including any suitable Cas9 or Cas9 equivalent.

### Wild Type Canonical SpCas9

In one embodiment, the base editor constructs described herein may comprise the “canonical SpCas9” nuclease from S. pyogenes, which has been widely used as a tool for genome engineering. This Cas9 protein is a large, multi-domain protein containing two distinct nuclease domains. Point mutations can be introduced into Cas9 to abolish one or both nuclease activities, resulting in a nickase Cas9 (nCas9) or dead Cas9 (dCas9), respectively, that still retains its ability to bind DNA in a sgRNA-programmed manner. In principle, when fused to another protein or domain, Cas9 or variant thereof (e.g., nCas9) can target that protein to virtually any DNA sequence simply by co-expression with an appropriate sgRNA. As used herein, the canonical SpCas9 protein refers to the wild type protein from Streptococcus pyogenes having the following amino acid sequence:

| | | SEQ |
|---|---|---|
| | | ID |
| Description | Sequence | NO: |
| SpCas 9 | MDKKYSIGLDIGTNSVGWAV | SEQ |
| Streptococcus | ITDEYKVPSKKFKVLGNTDR | ID |
| pyogenes | HSIKKNLIGALLFDSGETAE | NO: |
| M1 | ATRLKRTARRRYTRRKNRIC | 213 |
| SwissProt | YLQEIFSNEMAKVDDSFFHR | |
| Accession | LEESFLVEEDKKHERHPIFG | |
| No. Q99ZW2 | NIVDEVAYHEKYPTIYHLRK | |
| Wild type | KLVDSTDKADLRLIYLALAH | |
| | MIKFRGHFLIEGDLNPDNSD | |
| | VDKLFIQLVQTYNQLFEENP | |
| | INASGVDAKAILSARLSKSR | |
| | RLENLIAQLPGEKKNGLFGN | |
| | LIALSLGLTPNFKSNFDLAE | |
| | DAKLQLSKDTYDDDLDNLLA | |
| | QIGDQYADLFLAAKNLSDAI | |
| | LLSDILRVNTEITKAPLSAS | |
| | MIKRYDEHHQDLTLLKALVR | |
| | QQLPEKYKEIFFDQSKNGYA | |
| | GYIDGGASQEEFYKFIKPIL | |
| | EKMDGTEELLVKLNREDLLR | |
| | KQRTFDNGSIPHQIHLGELH | |
| | AILRRQEDFYPFLKDNREKI | |
| | EKILTFRIPYYVGPLARGNS | |
| | RFAWMTRKSEETITPWNFEE | |
| | VVDKGASAQSFIERMTNFDK | |
| | NLPNEKVLPKHSLLYEYFTV | |
| | YNELTKVKYVTEGMRKPAFL | |
| | SGEQKKAIVDLLFKTNRKVT | |
| | VKQLKEDYFKKIECFDSVEI | |
| | SGVEDRFNASLGTYHDLLKI | |
| | IKDKDFLDNEENEDILEDIV | |
| | LTLTLFEDREMIEERLKTYA | |
| | HLFDDKVMKQLKRRRYTGWG | |
| | RLSRKLINGIRDKQSGKTIL | |
| | DFLKSDGFANRNFMQLIHDD | |
| | SLTFKEDIQKAQVSGQGDSL | |
| | HEHIANLAGSPAIKKGILQT | |
| | VKVVDELVKVMGRHKPENIV | |
| | IEMARENQTTQKGQKNSRER | |
| | MKRIEEGIKELGSQILKEHP | |
| | VENTQLQNEKLYLYYLQNGR | |
| | DMYVDQELDINRLSDYDVDH | |
| | IVPQSFLKDDSIDNKVLTRS | |
| | DKNRGKSDNVPSEEVVKKMK | |
| | NYWRQLLNAKLITQRKFDNL | |
| | TKAERGGLSELDKAGFIKRQ | |
| | LVETRQITKHVAQILDSRMN | |
| | TKYDENDKLIREVKVITLKS | |
| | KLVSDFRKDFQFYKVREINN | |
| | YHHAHDAYLNAVVGTALIKK | |
| | YPKLESEFVYGDYKVYDVRK | |
| | MIAKSEQEIGKATAKYFFYS | |
| | NIMNFFKTEITLANGEIRKR | |
| | PLIETNGETGEIVWDKGRDF | |
| | ATVRKVLSMPQVNIVKKTEV | |
| | QTGGFSKESILPKRNSDKLI | |
| | ARKKDWDPKKYGGFDSPTVA | |
| | YSVLVVAKVEKGKSKKLKSV | |
| | KELLGITIMERSSFEKNPID | |
| | FLEAKGYKEVKKDLIIKLPK | |
| | YSLFELENGRKRMLASAGEL | |
| | QKGNELALPSKYVNFLYLAS | |
| | HYEKLKGSPEDNEQKQLFVE | |
| | QHKHYLDEIIEQISEFSKRV | |
| | ILADANLDKVLSAYNKHRDK | |
| | PIREQAENIIHLFTLTNLGA | |
| | PAAFKYFDTTIDRKRYTSTK | |
| | EVLDATLIHQSITGLYETRI | |
| | DLSQLGGD | |
| SpCas 9 | ATGGATAAAAAATATAGCAT | SEQ |
| Reverse | TGGCCTGGATATTGGCACCA | ID |
| translation | ACAGCGTGGGCTGGGCGGTG | NO: |
| of | ATTACCGATGAATATAAAGT | 1 |
| SwissProt | GCCGAGCAAAAAATTTAAAG | |
| Accession | TGCTGGGCAACACCGATCGC | |
| No. Q99ZW2 | CATAGCATTAAAAAAAACCT | |
| Streptococcus | GATTGGCGCGCTGCTGTTTG | |
| pyogenes | ATAGCGGCGAAACCGCGGAA | |
| | GCGACCCGCCTGAAACGCAC | |
| | CGCGCGCCGCCGCTATACCC | |
| | GCCGCAAAAACCGCATTTGC | |
| | TATCTGCAGGAAATTTTTAG | |
| | CAACGAAATGGCGAAAGTGG | |
| | ATGATAGCTTTTTTCATCGC | |
| | CTGGAAGAAAGCTTTCTGGT | |
| | GGAAGAAGATAAAAAACATG | |
| | AACGCCATCCGATTTTTGGC | |
| | AACATTGTGGATGAAGTGGC | |
| | GTATCATGAAAAATATCCGA | |
| | CCATTTATCATCTGCGCAAA | |
| | AAACTGGTGGATAGCACCGA | |
| | TAAAGCGGATCTGCGCCTGA | |
| | TTTATCTGGCGCTGGCGCAT | |
| | ATGATTAAATTTCGCGGCCA | |
| | TTTTCTGATTGAAGGCGATC | |
| | TGAACCCGGATAACAGCGAT | |
| | GTGGATAAACTGTTTATTCA | |
| | GCTGGTGCAGACCTATAACC | |
| | AGCTGTTTGAAGAAAACCCG | |
| | ATTAACGCGAGCGGCGTGGA | |
| | TGCGAAAGCGATTCTGAGCG | |
| | CGCGCCTGAGCAAAAGCCGC | |
| | CGCCTGGAAAACCTGATTGC | |
| | GCAGCTGCCGGGCGAAAAAA | |
| | AAAACGGCCTGTTTGGCAAC | |
| | CTGATTGCGCTGAGCCTGGG | |
| | CCTGACCCCGAACTTTAAAA | |
| | GCAACTTTGATCTGGCGGAA | |
| | GATGCGAAACTGCAGCTGAG | |
| | CAAAGATACCTATGATGATG | |
| | ATCTGGATAACCTGCTGGCG | |
| | CAGATTGGCGATCAGTATGC | |
| | GGATCTGTTTCTGGCGGCGA | |
| | AAAACCTGAGCGATGCGATT | |
| | CTGCTGAGCGATATTCTGCG | |
| | CGTGAACACCGAAATTACCA | |
| | AAGCGCCGCTGAGCGCGAGC | |
| | ATGATTAAACGCTATGATGA | |
| | ACATCATCAGGATCTGACCC | |
| | TGCTGAAAGCGCTGGTGCGC | |
| | CAGCAGCTGCCGGAAAAATA | |
| | TAAAGAAATTTTTTTTGATC | |
| | AGAGCAAAAACGGCTATGCG | |
| | GGCTATATTGATGGCGGCGC | |
| | GAGCCAGGAAGAATTTTATA | |
| | AATTTATTAAACCGATTCTG | |
| | GAAAAAATGGATGGCACCGA | |
| | AGAACTGCTGGTGAAACTGA | |
| | ACCGCGAAGATCTGCTGCGC | |
| | AAACAGCGCACCTTTGATAA | |
| | CGGCAGCATTCCGCATCAGA | |
| | TTCATCTGGGCGAACTGCAT | |
| | GCGATTCTGCGCCGCCAGGA | |
| | AGATTTTTATCCGTTTCTGA | |
| | AAGATAACCGCGAAAAAATT | |
| | GAAAAAATTCTGACCTTTCG | |
| | CATTCCGTATTATGTGGGCC | |
| | CGCTGGCGCGCGGCAACAGC | |
| | CGCTTTGCGTGGATGACCCG | |
| | CAAAAGCGAAGAAACCATTA | |
| | CCCCGTGGAACTTTGAAGAA | |
| | GTGGTGGATAAAGGCGCGAG | |
| | CGCGCAGAGCTTTATTGAAC | |
| | GCATGACCAACTTTGATAAA | |
| | AACCTGCCGAACGAAAAAGT | |
| | GCTGCCGAAACATAGCCTGC | |
| | TGTATGAATATTTTACCGTG | |
| | TATAACGAACTGACCAAAGT | |
| | GAAATATGTGACCGAAGGCA | |
| | TGCGCAAACCGGCGTTTCTG | |
| | AGCGGCGAACAGAAAAAAGC | |
| | GATTGTGGATCTGCTGTTTA | |
| | AAACCAACCGCAAAGTGACC | |
| | GTGAAACAGCTGAAAGAAGA | |
| | TTATTTTAAAAAAATTGAAT | |
| | GCTTTGATAGCGTGGAAATT | |
| | AGCGGCGTGGAAGATCGCTT | |
| | TAACGCGAGCCTGGGCACCT | |
| | ATCATGATCTGCTGAAAATT | |
| | ATTAAAGATAAAGATTTTCT | |
| | GGATAACGAAGAAAACGAAG | |
| | ATATTCTGGAAGATATTGTG | |
| | CTGACCCTGACCCTGTTTGA | |
| | AGATCGCGAAATGATTGAAG | |
| | AACGCCTGAAAACCTATGCG | |
| | CATCTGTTTGATGATAAAGT | |
| | GATGAAACAGCTGAAACGCC | |
| | GCCGCTATACCGGCTGGGGC | |
| | CGCCTGAGCCGCAAACTGAT | |
| | TAACGGCATTCGCGATAAAC | |
| | AGAGCGGCAAAACCATTCTG | |
| | GATTTTCTGAAAAGCGATGG | |
| | CTTTGCGAACCGCAACTTTA | |
| | TGCAGCTGATTCATGATGAT | |
| | AGCCTGACCTTTAAAGAAGA | |
| | TATTCAGAAAGCGCAGGTGA | |
| | GCGGCCAGGGCGATAGCCTG | |
| | CATGAACATATTGCGAACCT | |
| | GGCGGGCAGCCCGGCGATTA | |
| | AAAAAGGCATTCTGCAGACC | |
| | GTGAAAGTGGTGGATGAACT | |
| | GGTGAAAGTGATGGGCCGCC | |
| | ATAAACCGGAAAACATTGTG | |
| | ATTGAAATGGCGCGCGAAAA | |
| | CCAGACCACCCAGAAAGGCC | |
| | AGAAAAACAGCCGCGAACGC | |
| | ATGAAACGCATTGAAGAAGG | |
| | CATTAAAGAACTGGGCAGCC | |
| | AGATTCTGAAAGAACATCCG | |
| | GTGGAAAACACCCAGCTGCA | |
| | GAACGAAAAACTGTATCTGT | |
| | ATTATCTGCAGAACGGCCGC | |
| | GATATGTATGTGGATCAGGA | |
| | ACTGGATATTAACCGCCTGA | |
| | GCGATTATGATGTGGATCAT | |
| | ATTGTGCCGCAGAGCTTTCT | |
| | GAAAGATGATAGCATTGATA | |
| | ACAAAGTGCTGACCCGCAGC | |
| | GATAAAAACCGCGGCAAAAG | |
| | CGATAACGTGCCGAGCGAAG | |
| | AAGTGGTGAAAAAAATGAAA | |
| | AACTATTGGCGCCAGCTGCT | |
| | GAACGCGAAACTGATTACCC | |
| | AGCGCAAATTTGATAACCTG | |
| | ACCAAAGCGGAACGCGGCGG | |
| | CCTGAGCGAACTGGATAAAG | |
| | CGGGCTTTATTAAACGCCAG | |
| | CTGGTGGAAACCCGCCAGAT | |
| | TACCAAACATGTGGCGCAGA | |
| | TTCTGGATAGCCGCATGAAC | |
| | ACCAAATATGATGAAAACGA | |
| | TAAACTGATTCGCGAAGTGA | |
| | AAGTGATTACCCTGAAAAGC | |
| | AAACTGGTGAGCGATTTTCG | |
| | CAAAGATTTTCAGTTTTATA | |
| | AAGTGCGCGAAATTAACAAC | |
| | TATCATCATGCGCATGATGC | |
| | GTATCTGAACGCGGTGGTGG | |
| | GCACCGCGCTGATTAAAAAA | |
| | TATCCGAAACTGGAAAGCGA | |
| | ATTTGTGTATGGCGATTATA | |
| | AAGTGTATGATGTGCGCAAA | |
| | ATGATTGCGAAAAGCGAACA | |
| | GGAAATTGGCAAAGCGACCG | |
| | CGAAATATTTTTTTTATAGC | |
| | AACATTATGAACTTTTTTAA | |
| | AACCGAAATTACCCTGGCGA | |
| | ACGGCGAAATTCGCAAACGC | |
| | CCGCTGATTGAAACCAACGG | |
| | CGAAACCGGCGAAATTGTGT | |
| | GGGATAAAGGCCGCGATTTT | |
| | GCGACCGTGCGCAAAGTGCT | |
| | GAGCATGCCGCAGGTGAACA | |
| | TTGTGAAAAAAACCGAAGTG | |
| | CAGACCGGCGGCTTTAGCAA | |
| | AGAAAGCATTCTGCCGAAAC | |
| | GCAACAGCGATAAACTGATT | |
| | GCGCGCAAAAAAGATTGGGA | |
| | TCCGAAAAAATATGGCGGCT | |
| | TTGATAGCCCGACCGTGGCG | |
| | TATAGCGTGCTGGTGGTGGC | |
| | GAAAGTGGAAAAAGGCAAAA | |
| | GCAAAAAACTGAAAAGCGTG | |
| | AAAGAACTGCTGGGCATTAC | |
| | CATTATGGAACGCAGCAGCT | |
| | TTGAAAAAAACCCGATTGAT | |
| | TTTCTGGAAGCGAAAGGCTA | |
| | TAAAGAAGTGAAAAAAGATC | |
| | TGATTATTAAACTGCCGAAA | |
| | TATAGCCTGTTTGAACTGGA | |
| | AAACGGCCGCAAACGCATGC | |
| | TGGCGAGCGCGGGCGAACTG | |
| | CAGAAAGGCAACGAACTGGC | |
| | GCTGCCGAGCAAATATGTGA | |
| | ACTTTCTGTATCTGGCGAGC | |
| | CATTATGAAAAACTGAAAGG | |
| | CAGCCCGGAAGATAACGAAC | |
| | AGAAACAGCTGTTTGTGGAA | |
| | CAGCATAAACATTATCTGGA | |
| | TGAAATTATTGAACAGATTA | |
| | GCGAATTTAGCAAACGCGTG | |
| | ATTCTGGCGGATGCGAACCT | |
| | GGATAAAGTGCTGAGCGCGT | |
| | ATAACAAACATCGCGATAAA | |
| | CCGATTCGCGAACAGGCGGA | |
| | AAACATTATTCATCTGTTTA | |
| | CCCTGACCAACCTGGGCGCG | |
| | CCGGCGGCGTTTAAATATTT | |
| | TGATACCACCATTGATCGCA | |
| | AACGCTATACCAGCACCAAA | |
| | GAAGTGCTGGATGCGACCCT | |
| | GATTCATCAGAGCATTACCG | |
| | GCCTGTATGA | |
| | AACCCGCATTGATCTGAGCC | |
| | AGCTGGGCGGCGAT | |

The base editors described herein may include canonical SpCas9, or any variant thereof having at least 80%, at least 85%, at least 90%, at least 95%, or at least 99% sequence identity with a wild type Cas9 sequence provided above. These variants may include SpCas9 variants containing one or more mutations, including any known mutation reported with the SwissProt Accession No. Q99ZW2 entry, which include:

| SpCas9 mutation | |
|---|---|
| (relative to the | |
| amino acid sequence | |
| of the canonical | Function/Characteristic (as reported) (see |
| SpCas9 sequence, | UniProtKB—Q99ZW2 (CAS9_STRPT1) entry— |
| SEQ ID NO: 213) | incorporated herein by reference) |
| D10A | Nickase mutant which cleaves the protospacer |
| | strand (but no cleavage of non-protospacer |
| | strand) |
| S15A | Decreased DNA cleavage activity |
| R66A | Decreased DNA cleavage activity |
| R70A | No DNA cleavage |
| R74A | Decreased DNA cleavage |
| R78A | Decreased DNA cleavage |
| 97-150 deletion | No nuclease activity |
| R165A | Decreased DNA cleavage |
| 175-307 deletion | About 50% decreased DNA cleavage |
| 312-409 deletion | No nuclease activity |
| E762A | Nickase |
| H840A | Nickase mutant which cleaves the non- |
| | protospacer strand but does not cleave the |
| | protospacer strand |
| N854A | Nickase |
| N8 63A | Nickase |
| H982A | Decreased DNA cleavage |
| D986A | Nickase |
| 1099-1368 deletion | No nuclease activity |
| R1333A | Reduced DNA binding |

Other wild type SpCas9 sequences that may be used in the present disclosure, include:

| | | SEQ |
|---|---|---|
| | | ID |
| Description | Sequence | NO: |
| SpCas9 | ATGGATAAGAAATACTCAAT | SEQ |
| Streptococcus | AGGCTTAGATATCGGCACAA | ID |
| pyogenes | ATAGCGTCGGATGGGCGGTG | NO: |
| MGAS1882 wild | ATCACTGATGATTATAAGGT | 2 |
| type | TCCGTCTAAAAAGTTCAAGG | |
| NC_017053.1 | TTCTGGGAAATACAGACCGC | |
| | CACAGTATCAAAAAAAATCT | |
| | TATAGGGGCTCTTTTATTTG | |
| | GCAGTGGAGAGACAGCGGAA | |
| | GCGACTCGTCTCAAACGGAC | |
| | AGCTCGTAGAAGGTATACAC | |
| | GTCGGAAGAATCGTATTTGT | |
| | TATCTACAGGAGATTTTTTC | |
| | AAATGAGATGGCGAAAGTAG | |
| | ATGATAGTTTCTTTCATCGA | |
| | CTTGAAGAGTCTTTTTTGGT | |
| | GGAAGAAGACAAGAAGCATG | |
| | AACGTCATCCTATTTTTGGA | |
| | AATATAGTAGATGAAGTTGC | |
| | TTATCATGAGAAATATCCAA | |
| | CTATCTATCATCTGCGAAAA | |
| | AAATTGGCAGATTCTACTGA | |
| | TAAAGCGGATTTGCGCTTAA | |
| | TCTATTTGGCCTTAGCGCAT | |
| | ATGATTAAGTTTCGTGGTCA | |
| | TTTTTTGATTGAGGGAGATT | |
| | TAAATCCTGATAATAGTGAT | |
| | GTGGACAAACTATTTATCCA | |
| | GTTGGTACAAATCTACAATC | |
| | AATTATTTGAAGAAAACCCT | |
| | ATTAACGCAAGTAGAGTAGA | |
| | TGCTAAAGCGATTCTTTCTG | |
| | CACGATTGAGTAAATCAAGA | |
| | CGATTAGAAAATCTCATTGC | |
| | TCAGCTCCCCGGTGAGAAGA | |
| | GAAATGGCTTGTTTGGGAAT | |
| | CTCATTGCTTTGTCATTGGG | |
| | ATTGACCCCTAATTTTAAAT | |
| | CAAATTTTGATTTGGCAGAA | |
| | GATGCTAAATTACAGCTTTC | |
| | AAAAGATACTTACGATGATG | |
| | ATTTAGATAATTTATTGGCG | |
| | CAAATTGGAGATCAATATGC | |
| | TGATTTGTTTTTGGCAGCTA | |
| | AGAATTTATCAGATGCTATT | |
| | TTACTTTCAGATATCCTAAG | |
| | AGTAAATAGTGAAATAACTA | |
| | AGGCTCCCCTATCAGCTTCA | |
| | ATGATTAAGCGCTACGATGA | |
| | ACATCATCAAGACTTGACTC | |
| | TTTTAAAAGCTTTAGTTCGA | |
| | CAACAACTTCCAGAAAAGTA | |
| | TAAAGAAATCTTTTTTGATC | |
| | AATCAAAAAACGGATATGCA | |
| | GGTTATATTGATGGGGGAGO | |
| | TAGOCAAGAAGAATTTTATA | |
| | AATTTATCAAACCAATTTTA | |
| | GAAAAAATGGATGGTACTGA | |
| | GGAATTATTGGTGAAACTAA | |
| | ATCGTGAAGATTTGCTGCGC | |
| | AAGCAACGGACCTTTGACAA | |
| | CGGCTCTATTCCCCATCAAA | |
| | TTCACTTGGGTGAGCTGCAT | |
| | GCTATTTTGAGAAGACAAGA | |
| | AGACTTTTATCCATTTTTAA | |
| | AAGACAATCGTGAGAAGATT | |
| | GAAAAAATCTTGACTTTTCG | |
| | AATTCCTTATTATGTTGGTC | |
| | CATTGGCGCGTGGCAATAGT | |
| | CGTTTTGCATGGATGACTCG | |
| | GAAGTCTGAAGAAACAATTA | |
| | CCCCATGGAATTTTGAAGAA | |
| | GTTGTCGATAAAGGTGCTTC | |
| | AGCTCAATCATTTATTGAAC | |
| | GCATGACAAACTTTGATAAA | |
| | AATCTTCCAAATGAAAAAGT | |
| | ACTACCAAAACATAGTTTGC | |
| | TTTATGAGTATTTTACGGTT | |
| | TATAACGAATTGACAAAGGT | |
| | CAAATATGTTACTGAGGGAA | |
| | TGCGAAAACCAGCATTTCTT | |
| | TCAGGTGAACAGAAGAAAGC | |
| | CATTGTTGATTTACTCTTCA | |
| | AAACAAATCGAAAAGTAACC | |
| | GTTAAGCAATTAAAAGAAGA | |
| | TTATTTCAAAAAAATAGAAT | |
| | GTTTTGATAGTGTTGAAATT | |
| | TCAGGAGTTGAAGATAGATT | |
| | TAATGCTTCATTAGGCGCCT | |
| | ACCATGATTTGCTAAAAATT | |
| | ATTAAAGATAAAGATTTTTT | |
| | GGATAATGAAGAAAATGAAG | |
| | ATATCTTAGAGGATATTGTT | |
| | TTAACATTGACCTTATTTGA | |
| | AGATAGGGGGATGATTGAGG | |
| | AAAGACTTAAAACATATGCT | |
| | CACCTCTTTGATGATAAGGT | |
| | GATGAAACAGCTTAAACGTC | |
| | GCCGTTATACTGGTTGGGGA | |
| | CGTTTGTCTCGAAAATTGAT | |
| | TAATGGTATTAGGGATAAGC | |
| | AATCTGGCAAAACAATATTA | |
| | GATTTTTTGAAATCAGATGG | |
| | TTTTGCCAATCGCAATTTTA | |
| | TGCAGCTGATCCATGATGAT | |
| | AGTTTGACATTTAAAGAAGA | |
| | TATTCAAAAAGCACAGGTGT | |
| | CTGGACAAGGCCATAGTTTA | |
| | CATGAACAGATTGCTAACTT | |
| | AGCTGGCAGTCCTGCTATTA | |
| | AAAAAGGTATTTTACAGACT | |
| | GTAAAAATTGTTGATGAACT | |
| | GGTCAAAGTAATGGGGCATA | |
| | AGCCAGAAAATATCGTTATT | |
| | GAAATGGCACGTGAAAATCA | |
| | GACAACTCAAAAGGGCCAGA | |
| | AAAATTCGCGAGAGCGTATG | |
| | AAACGAATCGAAGAAGGTAT | |
| | CAAAGAATTAGGAAGTCAGA | |
| | TTCTTAAAGAGCATCCTGTT | |
| | GAAAATACTCAATTGCAAAA | |
| | TGAAAAGCTCTATCTCTATT | |
| | ATCTACAAAATGGAAGAGAC | |
| | ATGTATGTGGACCAAGAATT | |
| | AGATATTAATCGTTTAAGTG | |
| | ATTATGATGTCGATCACATT | |
| | GTTCCACAAAGTTTCATTAA | |
| | AGACGATTCAATAGACAATA | |
| | AGGTACTAACGCGTTCTGAT | |
| | AAAAATCGTGGTAAATCGGA | |
| | TAACGTTCCAAGTGAAGAAG | |
| | TAGTCAAAAAGATGAAAAAC | |
| | TATTGGAGACAACTTCTAAA | |
| | CGCCAAGTTAATCACTCAAC | |
| | GTAAGTTTGATAATTTAACG | |
| | AAAGCTGAACGTGGAGGTTT | |
| | GAGTGAACTTGATAAAGCTG | |
| | GTTTTATCAAACGCCAATTG | |
| | GTTGAAACTCGCCAAATCAC | |
| | TAAGCATGTGGCACAAATTT | |
| | TGGATAGTCGCATGAATACT | |
| | AAATACGATGAAAATGATAA | |
| | ACTTATTCGAGAGGTTAAAG | |
| | TGATTACCTTAAAATCTAAA | |
| | TTAGTTTCTGACTTCCGAAA | |
| | AGATTTCCAATTCTATAAAG | |
| | TACGTGAGATTAACAATTAC | |
| | CATCATGCCCATGATGCGTA | |
| | TCTAAATGCCGTCGTTGGAA | |
| | CTGCTTTGATTAAGAAATAT | |
| | CCAAAACTTGAATCGGAGTT | |
| | TGTCTATGGTGATTATAAAG | |
| | TTTATGATGTTCGTAAAATG | |
| | ATTGCTAAGTCTGAGCAAGA | |
| | AATAGGCAAAGCAACCGCAA | |
| | AATATTTCTTTTACTCTAAT | |
| | ATCATGAACTTCTTCAAAAC | |
| | AGAAATTACACTTGCAAATG | |
| | GAGAGATTCGCAAACGCCCT | |
| | CTAATCGAAACTAATGGGGA | |
| | AACTGGAGAAATTGTCTGGG | |
| | ATAAAGGGCGAGATTTTGCC | |
| | ACAGTGCGCAAAGTATTGTC | |
| | CATGCCCCAAGTCAATATTG | |
| | TCAAGAAAACAGAAGTACAG | |
| | ACAGGCGGATTCTCCAAGGA | |
| | GTCAATTTTACCAAAAAGAA | |
| | ATTCGGACAAGCTTATTGCT | |
| | CGTAAAAAAGACTGGGATCC | |
| | AAAAAAATATGGTGGTTTTG | |
| | ATAGTCCAACGGTAGCTTAT | |
| | TCAGTCCTAGTGGTTGCTAA | |
| | GGTGGAAAAAGGGAAATCGA | |
| | AGAAGTTAAAATCCGTTAAA | |
| | GAGTTACTAGGGATCACAAT | |
| | TATGGAAAGAAGTTCCTTTG | |
| | AAAAAAATCCGATTGACTTT | |
| | TTAGAAGCTAAAGGATATAA | |
| | GGAAGTTAAAAAAGACTTAA | |
| | TCATTAAACTACCTAAATAT | |
| | AGTCTTTTTGAGTTAGAAAA | |
| | CGGTCGTAAACGGATGCTGG | |
| | CTAGTGCCGGAGAATTACAA | |
| | AAAGGAAATGAGCTGGCTCT | |
| | GCCAAGCAAATATGTGAATT | |
| | TTTTATATTTAGCTAGTCAT | |
| | TATGAAAAGTTGAAGGGTAG | |
| | TCCAGAAGATAACGAACAAA | |
| | AACAATTGTTTGTGGAGCAG | |
| | CATAAGCATTATTTAGATGA | |
| | GATTATTGAGCAAATCAGTG | |
| | AATTTTCTAAGCGTGTTATT | |
| | TTAGCAGATGCCAATTTAGA | |
| | TAAAGTTCTTAGTGCATATA | |
| | ACAAACATAGAGACAAACCA | |
| | ATACGTGAACAAGCAGAAAA | |
| | TATTATTCATTTATTTACGT | |
| | TGACGAATCTTGGAGCTCCC | |
| | GCTGCTTTTAAATATTTTGA | |
| | TACAACAATTGATCGTAAAC | |
| | GATATACGTCTACAAAAGAA | |
| | GTTTTAGATGCCACTCTTAT | |
| | CCATCAATCCATCACTGGTC | |
| | TTTATGAAACACGCATTGAT | |
| | TTGAGTCAGCTAGGAGGTGA | |
| | CTGA | |
| SpCas9 | MDKKYSIGLDIGTNSVGWAV | SEQ |
| Streptococcus | ITDDYKVPSKKFKVLGNTDR | ID |
| pyogenes | HSIKKNLIGALLFGSGETAE | NO: |
| MGAS1882 wild | ATRLKRTARRRYTRRKNRIC | 3 |
| type | YLQEIFSNEMAKVDDSFFHR | |
| NC_017053.1 | LEESFLVEEDKKHERHPIFG | |
| | NIVDEVAYHEKYPTIYHLRK | |
| | KLADSTDKADLRLIYLALAH | |
| | MIKFRGHFLIEGDLNPDNSD | |
| | VDKLFIQLVQIYNQLFEENP | |
| | INASRVDAKAILSARLSKSR | |
| | RLENLIAQLPGEKRNGLFGN | |
| | LIALSLGLTPNFKSNFDLAE | |
| | DAKLQLSKDTYDDDLDNLLA | |
| | QIGDQYADLFLAAKNLSDAI | |
| | LLSDILRVNSEITKAPLSAS | |
| | MIKRYDEHHQDLTLLKALVR | |
| | QQLPEKYKEIFFDQSKNGYA | |
| | GYIDGGASQEEFYKFIKPIL | |
| | EKMDGTEELLVKLNREDLLR | |
| | KQRTFDNGSIPHQIHLGELH | |
| | AILRRQEDFYPFLKDNREKI | |
| | EKILTFRIPYYVGPLARGNS | |
| | RFAWMTRKSEETITPWNFEE | |
| | VVDKGASAQSFIERMTNFDK | |
| | NLPNEKVLPKHSLLYEYFTV | |
| | YNELTKVKYVTEGMRKPAFL | |
| | SGEQKKAIVDLLFKTNRKVT | |
| | VKQLKEDYFKKIECFDSVEI | |
| | SGVEDRFNASLGAYHDLLKI | |
| | IKDKDFLDNEENEDILEDIV | |
| | LTLTLFEDRGMIEERLKTYA | |
| | HLFDDKVMKQLKRRRYTGWG | |
| | RLSRKLINGIRDKQSGKTIL | |
| | DFLKSDGFANRNFMQLIHDD | |
| | SLTFKEDIQKAQVSGQGHSL | |
| | HEQIANLAGSPAIKKGILQT | |
| | VKIVDELVKVMGHKPENIVI | |
| | EMARENQTTQKGQKNSRERM | |
| | KRIEEGIKELGSQILKEHPV | |
| | ENTQLQNEKLYLYYLQNGRD | |
| | MYVDQELDINRLSDYDVDHI | |
| | VPQSFIKDDSIDNKVLTRSD | |
| | KNRGKSDNVPSEEVVKKMKN | |
| | YWRQLLNAKLITQRKFDNLT | |
| | KAERGGLSELDKAGFIKRQL | |
| | VETRQITKHVAQILDSRMNT | |
| | KYDENDKLIREVKVITLKSK | |
| | LVSDFRKDFQFYKVREINNY | |
| | HHAHDAYLNAVVGTALIKKY | |
| | PKLESEFVYGDYKVYDVRKM | |
| | IAKSEQEIGKATAKYFFYSN | |
| | IMNFFKTEITLANGEIRKRP | |
| | LIETNGETGEIVWDKGRDFA | |
| | TVRKVLSMPQVNIVKKTEVQ | |
| | TGGFSKESILPKRNSDKLIA | |
| | RKKDWDPKKYGGFDSPTVAY | |
| | SVLVVAKVEKGKSKKLKSVK | |
| | ELLGITIMERSSFEKNPIDF | |
| | LEAKGYKEVKKDLIIKLPKY | |
| | SLFELENGRKRMLASAGELQ | |
| | KGNELALPSKYVNFLYLASH | |
| | YEKLKGSPEDNEQKQLFVEQ | |
| | HKHYLDEIIEQISEFSKRVI | |
| | LADANLDKVLSAYNKHRDKP | |
| | IREQAENIIHLFTLTNLGAP | |
| | AAFKYFDTTIDRKRYTSTKE | |
| | VLDATLIHQSITGLYETRID | |
| | LSQLGGD | |
| SpCas9 | ATGGATAAAAAGTATTCTAT | SEQ |
| Streptococcus | TGGTTTAGACATCGGCACTA | ID |
| pyogenes wild | ATTCCGTTGGATGGGCTGTC | NO: |
| type | ATAACCGATGAATACAAAGT | 4 |
| SWBC2D7W014 | ACCTTCAAAGAAATTTAAGG | |
| | TGTTGGGGAACACAGACCGT | |
| | CATTCGATTAAAAAGAATCT | |
| | TATCGGTGCCCTCCTATTCG | |
| | ATAGTGGCGAAACGGCAGAG | |
| | GCGACTCGCCTGAAACGAAC | |
| | CGCTCGGAGAAGGTATACAC | |
| | GTCGCAAGAACCGAATATGT | |
| | TACTTACAAGAAATTTTTAG | |
| | CAATGAGATGGCCAAAGTTG | |
| | ACGATTCTTTCTTTCACCGT | |
| | TTGGAAGAGTCCTTCCTTGT | |
| | CGAAGAGGACAAGAAACATG | |
| | AACGGCACCCCATCTTTGGA | |
| | AACATAGTAGATGAGGTGGC | |
| | ATATCATGAAAAGTACCCAA | |
| | CGATTTATCACCTCAGAAAA | |
| | AAGCTAGTTGACTCAACTGA | |
| | TAAAGCGGACCTGAGGTTAA | |
| | TCTACTTGGCTCTTGCCCAT | |
| | ATGATAAAGTTCCGTGGGCA | |
| | CTTTCTCATTGAGGGTGATC | |
| | TAAATCCGGACAACTCGGAT | |
| | GTCGACAAACTGTTCATCCA | |
| | GTTAGTACAAACCTATAATC | |
| | AGTTGTTTGAAGAGAACCCT | |
| | ATAAATGCAAGTGGCGTGGA | |
| | TGCGAAGGCTATTCTTAGCG | |
| | CCCGCCTCTCTAAATCCCGA | |
| | CGGCTAGAAAACCTGATCGC | |
| | ACAATTACCCGGAGAGAAGA | |
| | AAAATGGGTTGTTCGGTAAC | |
| | CTTATAGCGCTCTCACTAGG | |
| | CCTGACACCAAATTTTAAGT | |
| | CGAACTTCGACTTAGCTGAA | |
| | GATGCCAAATTGCAGCTTAG | |
| | TAAGGACACGTACGATGACG | |
| | ATCTCGACAATCTACTGGCA | |
| | CAAATTGGAGATCAGTATGC | |
| | GGACTTATTTTTGGCTGCCA | |
| | AAAACCTTAGCGATGCAATC | |
| | CTCCTATCTGACATACTGAG | |
| | AGTTAATACTGAGATTACCA | |
| | AGGCGCCGTTATCCGCTTCA | |
| | ATGATCAAAAGGTACGATGA | |
| | ACATCACCAAGACTTGACAC | |
| | TTCTCAAGGCCCTAGTCCGT | |
| | CAGCAACTGCCTGAGAAATA | |
| | TAAGGAAATATTCTTTGATC | |
| | AGTCGAAAAACGGGTACGCA | |
| | GGTTATATTGACGGCGGAGC | |
| | GAGTCAAGAGGAATTCTACA | |
| | AGTTTATCAAACCCATATTA | |
| | GAGAAGATGGATGGGACGGA | |
| | AGAGTTGCTTGTAAAACTCA | |
| | ATCGCGAAGATCTACTGCGA | |
| | AAGCAGCGGACTTTCGACAA | |
| | CGGTAGCATTCCACATCAAA | |
| | TCCACTTAGGCGAATTGCAT | |
| | GCTATACTTAGAAGGCAGGA | |
| | GGATTTTTATCCGTTCCTCA | |
| | AAGACAATCGTGAAAAGATT | |
| | GAGAAAATCCTAACCTTTCG | |
| | CATACCTTACTATGTGGGAC | |
| | CCCTGGCCCGAGGGAACTCT | |
| | CGGTTCGCATGGATGACAAG | |
| | AAAGTCCGAAGAAACGATTA | |
| | CTCCATGGAATTTTGAGGAA | |
| | GTTGTCGATAAAGGTGCGTC | |
| | AGCTCAATCGTTCATCGAGA | |
| | GGATGACCAACTTTGACAAG | |
| | AATTTACCGAACGAAAAAGT | |
| | ATTGCCTAAGCACAGTTTAC | |
| | TTTACGAGTATTTCACAGTG | |
| | TACAATGAACTCACGAAAGT | |
| | TAAGTATGTCACTGAGGGCA | |
| | TGCGTAAACCCGCCTTTCTA | |
| | AGCGGAGAACAGAAGAAAGC | |
| | AATAGTAGATCTGTTATTCA | |
| | AGACCAACCGCAAAGTGACA | |
| | GTTAAGCAATTGAAAGAGGA | |
| | CTACTTTAAGAAAATTGAAT | |
| | GCTTCGATTCTGTCGAGATC | |
| | TCCGGGGTAGAAGATCGATT | |
| | TAATGCGTCACTTGGTACGT | |
| | ATCATGACCTCCTAAAGATA | |
| | ATTAAAGATAAGGACTTCCT | |
| | GGATAACGAAGAGAATGAAG | |
| | ATATCTTAGAAGATATAGTG | |
| | TTGACTCTTACCCTCTTTGA | |
| | AGATCGGGAAATGATTGAGG | |
| | AAAGACTAAAAACATACGCT | |
| | CACCTGTTCGACGATAAGGT | |
| | TATGAAACAGTTAAAGAGGC | |
| | GTCGCTATACGGGCTGGGGA | |
| | CGATTGTCGCGGAAACTTAT | |
| | CAACGGGATAAGAGACAAGC | |
| | AAAGTGGTAAAACTATTCTC | |
| | GATTTTCTAAAGAGCGACGG | |
| | CTTCGCCAATAGGAACTTTA | |
| | TGCAGCTGATCCATGATGAC | |
| | TCTTTAACCTTCAAAGAGGA | |
| | TATACAAAAGGCACAGGTTT | |
| | CCGGACAAGGGGACTCATTG | |
| | CACGAACATATTGCGAATCT | |
| | TGCTGGTTCGCCAGCCATCA | |
| | AAAAGGGCATACTCCAGACA | |
| | GTCAAAGTAGTGGATGAGCT | |
| | AGTTAAGGTCATGGGACGTC | |
| | ACAAACCGGAAAACATTGTA | |
| | ATCGAGATGGCACGCGAAAA | |
| | TCAAACGACTCAGAAGGGGC | |
| | AAAAAAACAGTCGAGAGCGG | |
| | ATGAAGAGAATAGAAGAGGG | |
| | TATTAAAGAACTGGGCAGCC | |
| | AGATCTTAAAGGAGCATCCT | |
| | GTGGAAAATACCCAATTGCA | |
| | GAACGAGAAACTTTACCTCT | |
| | ATTACCTACAAAATGGAAGG | |
| | GACATGTATGTTGATCAGGA | |
| | ACTGGACATAAACCGTTTAT | |
| | CTGATTACGACGTCGATCAC | |
| | ATTGTACCCCAATCCTTTTT | |
| | GAAGGACGATTCAATCGACA | |
| | ATAAAGTGCTTACACGCTCG | |
| | GATAAGAACCGAGGGAAAAG | |
| | TGACAATGTTCCAAGCGAGG | |
| | AAGTCGTAAAGAAAATGAAG | |
| | AACTATTGGCGGCAGCTCCT | |
| | AAATGCGAAACTGATAACGC | |
| | AAAGAAAGTTCGATAACTTA | |
| | ACTAAAGCTGAGAGGGGTGG | |
| | CTTGTCTGAACTTGACAAGG | |
| | CCGGATTTATTAAACGTCAG | |
| | CTCGTGGAAACCCGCCAAAT | |
| | CACAAAGCATGTTGCACAGA | |
| | TACTAGATTCCCGAATGAAT | |
| | ACGAAATACGACGAGAACGA | |
| | TAAGCTGATTCGGGAAGTCA | |
| | AAGTAATCACTTTAAAGTCA | |
| | AAATTGGTGTCGGACTTCAG | |
| | AAAGGATTTTCAATTCTATA | |
| | AAGTTAGGGAGATAAATAAC | |
| | TACCACCATGCGCACGACGC | |
| | TTATCTTAATGCCGTCGTAG | |
| | GGACCGCACTCATTAAGAAA | |
| | TACCCGAAGCTAGAAAGTGA | |
| | GTTTGTGTATGGTGATTACA | |
| | AAGTTTATGACGTCCGTAAG | |
| | ATGATCGCGAAAAGCGAACA | |
| | GGAGATAGGCAAGGCTACAG | |
| | CCAAATACTTCTTTTATTCT | |
| | AACATTATGAATTTCTTTAA | |
| | GACGGAAATCACTCTGGCAA | |
| | ACGGAGAGATACGCAAACGA | |
| | CCTTTAATTGAAACCAATGG | |
| | GGAGACAGGTGAAATCGTAT | |
| | GGGATAAGGGCCGGGACTTC | |
| | GCGACGGTGAGAAAAGTTTT | |
| | GTCCATGCCCCAAGTCAACA | |
| | TAGTAAAGAAAACTGAGGTG | |
| | CAGACCGGAGGGTTTTCAAA | |
| | GGAATCGATTCTTCCAAAAA | |
| | GGAATAGTGATAAGCTCATC | |
| | GCTCGTAAAAAGGACTGGGA | |
| | CCCGAAAAAGTACGGTGGCT | |
| | TCGATAGCCCTACAGTTGCC | |
| | TATTCTGTCCTAGTAGTGGC | |
| | AAAAGTTGAGAAGGGAAAAT | |
| | CCAAGAAACTGAAGTCAGTC | |
| | AAAGAATTATTGGGGATAAC | |
| | GATTATGGAGCGCTCGTCTT | |
| | TTGAAAAGAACCCCATCGAC | |
| | TTCCTTGAGGCGAAAGGTTA | |
| | CAAGGAAGTAAAAAAGGATC | |
| | TCATAATTAAACTACCAAAG | |
| | TATAGTCTGTTTGAGTTAGA | |
| | AAATGGCCGAAAACGGATGT | |
| | TGGCTAGCGCCGGAGAGCTT | |
| | CAAAAGGGGAACGAACTCGC | |
| | ACTACCGTCTAAATACGTGA | |
| | ATTTCCTGTATTTAGCGTCC | |
| | CATTACGAGAAGTTGAAAGG | |
| | TTCACCTGAAGATAACGAAC | |
| | AGAAGCAACTTTTTGTTGAG | |
| | CAGCACAAACATTATCTCGA | |
| | CGAAATCATAGAGCAAATTT | |
| | CGGAATTCAGTAAGAGAGTC | |
| | ATCCTAGCTGATGCCAATCT | |
| | GGACAAAGTATTAAGCGCAT | |
| | ACAACAAGCACAGGGATAAA | |
| | CCCATACGTGAGCAGGCGGA | |
| | AAATATTATCCATTTGTTTA | |
| | CTCTTACCAACCTCGGCGCT | |
| | CCAGCCGCATTCAAGTATTT | |
| | TGACACAACGATAGATCGCA | |
| | AACGATACACTTCTACCAAG | |
| | GAGGTGCTAGACGCGACACT | |
| | GATTCACCAATCCATCACGG | |
| | GATTATATGAAACTCGGATA | |
| | GATTTGTCACAGCTTGGGGG | |
| | TGACGGATCCCCCAAGAAGA | |
| | AGAGGAAAGTCTCGAGCGAC | |
| | TACAAAGACCATGACGGTGA | |
| | TTATAAAGATCATGACATCG | |
| | ATTACAAGGATGACGATGAC | |
| | AAGGCTGCAGGA | |
| SpCas9 | MDKKYSIGLDIGTNSVGWAV | SEQ |
| Streptococcus | ITDEYKVPSKKFKVLGNTDR | ID |
| pyogenes wild | HSIKKNLIGALLFDSGETAE | NO: |
| type | ATRLKRTARRRYTRRKNRIC | 5 |
| Encoded | YLQEIFSNEMAKVDDSFFHR | |
| product of | LEESFLVEEDKKHERHPIFG | |
| SWBC2D7WO14 | NIVDEVAYHEKYPTIYHLRK | |
| | KLVDSTDKADLRLIYLALAH | |
| | MIKFRGHFLIEGDLNPDNSD | |
| | VDKLFIQLVQTYNQLFEENP | |
| | INASGVDAKAILSARLSKSR | |
| | RLENLIAQLPGEKKNGLFGN | |
| | LIALSLGLTPNFKSNFDLAE | |
| | DAKLQLSKDTYDDDLDNLLA | |
| | QIGDQYADLFLAAKNLSDAI | |
| | LLSDILRVNTEITKAPLSAS | |
| | MIKRYDEHHQDLTLLKALVR | |
| | QQLPEKYKEIFFDQSKNGYA | |
| | GYIDGGASQEEFYKFIKPIL | |
| | EKMDGTEELLVKLNREDLLR | |
| | KQRTFDNGSIPHQIHLGELH | |
| | AILRRQEDFYPFLKDNREKI | |
| | EKILTFRIPYYVGPLARGNS | |
| | RFAWMTRKSEETITPWNFEE | |
| | VVDKGASAQSFIERMTNFDK | |
| | NLPNEKVLPKHSLLYEYFTV | |
| | YNELTKVKYVTEGMRKPAFL | |
| | SGEQKKAIVDLLFKTNRKVT | |
| | VKQLKEDYFKKIECFDSVEI | |
| | SGVEDRFNASLGTYHDLLKI | |
| | IKDKDFLDNEENEDILEDIV | |
| | LTLTLFEDREMIEERLKTYA | |
| | HLFDDKVMKQLKRRRYTGWG | |
| | RLSRKLINGIRDKQSGKTIL | |
| | DFLKSDGFANRNFMQLIHDD | |
| | SLTFKEDIQKAQVSGQGDSL | |
| | HEHIANLAGSPAIKKGILQT | |
| | VKVVDELVKVMGRHKPENIV | |
| | IEMARENQTTQKGQKNSRER | |
| | MKRIEEGIKELGSQILKEHP | |
| | VENTQLQNEKLYLYYLQNGR | |
| | DMYVDQELDINRLSDYDVDH | |
| | IVPQSFLKDDSIDNKVLTRS | |
| | DKNRGKSDNVPSEEVVKKMK | |
| | NYWRQLLNAKLITQRKFDNL | |
| | TKAERGGLSELDKAGFIKRQ | |
| | LVETRQITKHVAQILDSRMN | |
| | TKYDENDKLIREVKVITLKS | |
| | KLVSDFRKDFQFYKVREINN | |
| | YHHAHDAYLNAVVGTALIKK | |
| | YPKLESEFVYGDYKVYDVRK | |
| | MIAKSEQEIGKATAKYFFYS | |
| | NIMNFFKTEITLANGEIRKR | |
| | PLIETNGETGEIVWDKGRDF | |
| | ATVRKVLSMPQVNIVKKTEV | |
| | QTGGFSKESILPKRNSDKLI | |
| | ARKKDWDPKKYGGFDSPTVA | |
| | YSVLVVAKVEKGKSKKLKSV | |
| | KELLGITIMERSSFEKNPID | |
| | FLEAKGYKEVKKDLIIKLPK | |
| | YSLFELENGRKRMLASAGEL | |
| | QKGNELALPSKYVNFLYLAS | |
| | HYEKLKGSPEDNEQKQLFVE | |
| | QHKHYLDEIIEQISEFSKRV | |
| | ILADANLDKVLSAYNKHRDK | |
| | PIREQAENIIHLFTLTNLGA | |
| | PAAFKYFDTTIDRKRYTSTK | |
| | EVLDATLIHQSITGLYETRI | |
| | DLSQLGGDGSPKKKRKVSSD | |
| | YKDHDGDYKDHDIDYKDDDD | |
| | KAAG | |
| SpCas9 | ATGGATAAGAAATACTCAAT | SEQ |
| Streptococcus | AGGCTTAGATATCGGCACAA | ID |
| pyogenes | ATAGCGTCGGATGGGCGGTG | NO: |
| M1GAS wild | ATCACTGATGAATATAAGGT | 6 |
| type | TCCGTCTAAAAAGTTCAAGG | |
| NC_002737.2 | TTCTGGGAAATACAGACCGC | |
| | CACAGTATCAAAAAAAATCT | |
| | TATAGGGGCTCTTTTATTTG | |
| | ACAGTGGAGAGACAGCGGAA | |
| | GCGACTCGTCTCAAACGGAC | |
| | AGCTCGTAGAAGGTATACAC | |
| | GTCGGAAGAATCGTATTTGT | |
| | TATCTACAGGAGATTTTTTC | |
| | AAATGAGATGGCGAAAGTAG | |
| | ATGATAGTTTCTTTCATCGA | |
| | CTTGAAGAGTCTTTTTTGGT | |
| | GGAAGAAGACAAGAAGCATG | |
| | AACGTCATCCTATTTTTGGA | |
| | AATATAGTAGATGAAGTTGC | |
| | TTATCATGAGAAATATCCAA | |
| | CTATCTATCATCTGCGAAAA | |
| | AAATTGGTAGATTCTACTGA | |
| | TAAAGCGGATTTGCGCTTAA | |
| | TCTATTTGGCCTTAGCGCAT | |
| | ATGATTAAGTTTCGTGGTCA | |
| | TTTTTTGATTGAGGGAGATT | |
| | TAAATCCTGATAATAGTGAT | |
| | GTGGACAAACTATTTATCCA | |
| | GTTGGTACAAACCTACAATC | |
| | AATTATTTGAAGAAAACCCT | |
| | ATTAACGCAAGTGGAGTAGA | |
| | TGCTAAAGCGATTCTTTCTG | |
| | CACGATTGAGTAAATCAAGA | |
| | CGATTAGAAAATCTCATTGC | |
| | TCAGCTCCCCGGTGAGAAGA | |
| | AAAATGGCTTATTTGGGAAT | |
| | CTCATTGCTTTGTCATTGGG | |
| | TTTGACCCCTAATTTTAAAT | |
| | CAAATTTTGATTTGGCAGAA | |
| | GATGCTAAATTACAGCTTTC | |
| | AAAAGATACTTACGATGATG | |
| | ATTTAGATAATTTATTGGCG | |
| | CAAATTGGAGATCAATATGC | |
| | TGATTTGTTTTTGGCAGCTA | |
| | AGAATTTATCAGATGCTATT | |
| | TTACTTTCAGATATCCTAAG | |
| | AGTAAATACTGAAATAACTA | |
| | AGGCTCCCCTATCAGCTTCA | |
| | ATGATTAAACGCTACGATGA | |
| | ACATCATCAAGACTTGACTC | |
| | TTTTAAAAGCTTTAGTTCGA | |
| | CAACAACTTCCAGAAAAGTA | |
| | TAAAGAAATCTTTTTTGATC | |
| | AATCAAAAAACGGATATGCA | |
| | GGTTATATTGATGGGGGAGO | |
| | TAGOCAAGAAGAATTTTATA | |
| | AATTTATCAAACCAATTTTA | |
| | GAAAAAATGGATGGTACTGA | |
| | GGAATTATTGGTGAAACTAA | |
| | ATCGTGAAGATTTGCTGCGC | |
| | AAGCAACGGACCTTTGACAA | |
| | CGGCTCTATTCCCCATCAAA | |
| | TTCACTTGGGTGAGCTGCAT | |
| | GCTATTTTGAGAAGACAAGA | |
| | AGACTTTTATCCATTTTTAA | |
| | AAGACAATCGTGAGAAGATT | |
| | GAAAAAATCTTGACTTTTCG | |
| | AATTCCTTATTATGTTGGTC | |
| | CATTGGCGCGTGGCAATAGT | |
| | CGTTTTGCATGGATGACTCG | |
| | GAAGTCTGAAGAAACAATTA | |
| | CCCCATGGAATTTTGAAGAA | |
| | GTTGTCGATAAAGGTGCTTC | |
| | AGCTCAATCATTTATTGAAC | |
| | GCATGACAAACTTTGATAAA | |
| | AATCTTCCAAATGAAAAAGT | |
| | ACTACCAAAACATAGTTTGC | |
| | TTTATGAGTATTTTACGGTT | |
| | TATAACGAATTGACAAAGGT | |
| | CAAATATGTTACTGAAGGAA | |
| | TGCGAAAACCAGCATTTCTT | |
| | TCAGGTGAACAGAAGAAAGC | |
| | CATTGTTGATTTACTCTTCA | |
| | AAACAAATCGAAAAGTAACC | |
| | GTTAAGCAATTAAAAGAAGA | |
| | TTATTTCAAAAAAATAGAAT | |
| | GTTTTGATAGTGTTGAAATT | |
| | TCAGGAGTTGAAGATAGATT | |
| | TAATGCTTCATTAGGTACCT | |
| | ACCATGATTTGCTAAAAATT | |
| | ATTAAAGATAAAGATTTTTT | |
| | GGATAATGAAGAAAATGAAG | |
| | ATATCTTAGAGGATATTGTT | |
| | TTAACATTGACCTTATTTGA | |
| | AGATAGGGAGATGATTGAGG | |
| | AAAGACTTAAAACATATGCT | |
| | CACCTCTTTGATGATAAGGT | |
| | GATGAAACAGCTTAAACGTC | |
| | GCCGTTATACTGGTTGGGGA | |
| | CGTTTGTCTCGAAAATTGAT | |
| | TAATGGTATTAGGGATAAGC | |
| | AATCTGGCAAAACAATATTA | |
| | GATTTTTTGAAATCAGATGG | |
| | TTTTGCCAATCGCAATTTTA | |
| | TGCAGCTGATCCATGATGAT | |
| | AGTTTGACATTTAAAGAAGA | |
| | CATTCAAAAAGCACAAGTGT | |
| | CTGGACAAGGCGATAGTTTA | |
| | CATGAACATATTGCAAATTT | |
| | AGCTGGTAGCCCTGCTATTA | |
| | AAAAAGGTATTTTACAGACT | |
| | GTAAAAGTTGTTGATGAATT | |
| | GGTCAAAGTAATGGGGCGGC | |
| | ATAAGCCAGAAAATATCGTT | |
| | ATTGAAATGGCACGTGAAAA | |
| | TCAGACAACTCAAAAGGGCC | |
| | AGAAAAATTCGCGAGAGCGT | |
| | ATGAAACGAATCGAAGAAGG | |
| | TATCAAAGAATTAGGAAGTC | |
| | AGATTCTTAAAGAGCATCCT | |
| | GTTGAAAATACTCAATTGCA | |
| | AAATGAAAAGCTCTATCTCT | |
| | ATTATCTCCAAAATGGAAGA | |
| | GACATGTATGTGGACCAAGA | |
| | ATTAGATATTAATCGTTTAA | |
| | GTGATTATGATGTCGATCAC | |
| | ATTGTTCCACAAAGTTTCCT | |
| | TAAAGACGATTCAATAGACA | |
| | ATAAGGTCTTAACGCGTTCT | |
| | GATAAAAATCGTGGTAAATC | |
| | GGATAACGTTCCAAGTGAAG | |
| | AAGTAGTCAAAAAGATGAAA | |
| | AACTATTGGAGACAACTTCT | |
| | AAACGCCAAGTTAATCACTC | |
| | AACGTAAGTTTGATAATTTA | |
| | ACGAAAGCTGAACGTGGAGG | |
| | TTTGAGTGAACTTGATAAAG | |
| | CTGGTTTTATCAAACGCCAA | |
| | TTGGTTGAAACTCGCCAAAT | |
| | CACTAAGCATGTGGCACAAA | |
| | TTTTGGATAGTCGCATGAAT | |
| | ACTAAATACGATGAAAATGA | |
| | TAAACTTATTCGAGAGGTTA | |
| | AAGTGATTACCTTAAAATCT | |
| | AAATTAGTTTCTGACTTCCG | |
| | AAAAGATTTCCAATTCTATA | |
| | AAGTACGTGAGATTAACAAT | |
| | TACCATCATGCCCATGATGC | |
| | GTATCTAAATGCCGTCGTTG | |
| | GAACTGCTTTGATTAAGAAA | |
| | TATCCAAAACTTGAATCGGA | |
| | GTTTGTCTATGGTGATTATA | |
| | AAGTTTATGATGTTCGTAAA | |
| | ATGATTGCTAAGTCTGAGCA | |
| | AGAAATAGGCAAAGCAACCG | |
| | CAAAATATTTCTTTTACTCT | |
| | AATATCATGAACTTCTTCAA | |
| | AACAGAAATTACACTTGCAA | |
| | ATGGAGAGATTCGCAAACGC | |
| | CCTCTAATCGAAACTAATGG | |
| | GGAAACTGGAGAAATTGTCT | |
| | GGGATAAAGGGCGAGATTTT | |
| | GCCACAGTGCGCAAAGTATT | |
| | GTCCATGCCCCAAGTCAATA | |
| | TTGTCAAGAAAACAGAAGTA | |
| | CAGACAGGCGGATTCTCCAA | |
| | GGAGTCAATTTTACCAAAAA | |
| | GAAATTCGGACAAGCTTATT | |
| | GCTCGTAAAAAAGACTGGGA | |
| | TCCAAAAAAATATGGTGGTT | |
| | TTGATAGTCCAACGGTAGCT | |
| | TATTCAGTCCTAGTGGTTGC | |
| | TAAGGTGGAAAAAGGGAAAT | |
| | CGAAGAAGTTAAAATCCGTT | |
| | AAAGAGTTACTAGGGATCAC | |
| | AATTATGGAAAGAAGTTCCT | |
| | TTGAAAAAAATCCGATTGAC | |
| | TTTTTAGAAGCTAAAGGATA | |
| | TAAGGAAGTTAAAAAAGACT | |
| | TAATCATTAAACTACCTAAA | |
| | TATAGTCTTTTTGAGTTAGA | |
| | AAACGGTCGTAAACGGATGC | |
| | TGGCTAGTGCCGGAGAATTA | |
| | CAAAAAGGAAATGAGCTGGC | |
| | TCTGCCAAGCAAATATGTGA | |
| | ATTTTTTATATTTAGCTAGT | |
| | CATTATGAAAAGTTGAAGGG | |
| | TAGTCCAGAAGATAACGAAC | |
| | AAAAACAATTGTTTGTGGAG | |
| | CAGCATAAGCATTATTTAGA | |
| | TGAGATTATTGAGCAAATCA | |
| | GTGAATTTTCTAAGCGTGTT | |
| | ATTTTAGCAGATGCCAATTT | |
| | AGATAAAGTTCTTAGTGCAT | |
| | ATAACAAACATAGAGACAAA | |
| | CCAATACGTGAACAAGCAGA | |
| | AAATATTATTCATTTATTTA | |
| | CGTTGACGAATCTTGGAGCT | |
| | CCCGCTGCTTTTAAATATTT | |
| | TGATACAACAATTGATCGTA | |
| | AACGATATACGTCTACAAAA | |
| | GAAGTTTTAGATGCCACTCT | |
| | TATCCATCAATCCATCACTG | |
| | GTCTTTATGAAACACGCATT | |
| | GATTTGAGTCAGCTAGGAGG | |
| | TGACTGA | |
| SpCas9 | MDKKYSIGLDIGTNSVGWAV | |
| Streptococcus | ITDEYKVPSKKFKVLGNTDR | |
| pyogenes | HSIKKNLIGALLFDSGETAE | |
| M1GAS wild | ATRLKRTARRRYTRRKNRIC | |
| type | YLQEIFSNEMAKVDDSFFHR | |
| Encoded | LEESFLVEEDKKHERHPIFG | |
| product of | NIVDEVAYHEKYPTIYHLRK | |
| NC_002737.2 | KLVDSTDKADLRLIYLALAH | |
| (100% | MIKFRGHFLIEGDLNPDNSD | |
| identical to | VDKLFIQLVQTYNQLFEENP | |
| the canonical | INASGVDAKAILSARLSKSR | |
| Q99ZW2 | RLENLIAQLPGEKKNGLFGN | |
| wild type) | LIALSLGLTPNFKSNFDLAE | |
| | DAKLQLSKDTYDDDLDNLLA | |
| | QIGDQYADLFLAAKNLSDAI | |
| | LLSDILRVNTEITKAPLSAS | |
| | MIKRYDEHHQDLTLLKALVR | |
| | QQLPEKYKEIFFDQSKNGYA | |
| | GYIDGGASQEEFYKFIKPIL | |
| | EKMDGTEELLVKLNREDLLR | |
| | KQRTFDNGSIPHQIHLGELH | |
| | AILRRQEDFYPFLKDNREKI | |
| | EKILTFRIPYYVGPLARGNS | |
| | RFAWMTRKSEETITPWNFEE | |
| | VVDKGASAQSFIERMTNFDK | |
| | NLPNEKVLPKHSLLYEYFTV | |
| | YNELTKVKYVTEGMRKPAFL | |
| | SGEQKKAIVDLLFKTNRKVT | |
| | VKQLKEDYFKKIECFDSVEI | |
| | SGVEDRFNASLGTYHDLLKI | |
| | IKDKDFLDNEENEDILEDIV | |
| | LTLTLFEDREMIEERLKTYA | |
| | HLFDDKVMKQLKRRRYTGWG | |
| | RLSRKLINGIRDKQSGKTIL | |
| | DFLKSDGFANRNFMQLIHDD | |
| | SLTFKEDIQKAQVSGQGDSL | |
| | HEHIANLAGSPAIKKGILQT | |
| | VKVVDELVKVMGRHKPENIV | |
| | IEMARENQTTQKGQKNSRER | |
| | MKRIEEGIKELGSQILKEHP | |
| | VENTQLQNEKLYLYYLQNGR | |
| | DMYVDQELDINRLSDYDVDH | |
| | IVPQSFLKDDSIDNKVLTRS | |
| | DKNRGKSDNVPSEEVVKKMK | |
| | NYWRQLLNAKLITQRKFDNL | |
| | TKAERGGLSELDKAGFIKRQ | |
| | LVETRQITKHVAQILDSRMN | |
| | TKYDENDKLIREVKVITLKS | |
| | KLVSDFRKDFQFYKVREINN | |
| | YHHAHDAYLNAVVGTALIKK | |
| | YPKLESEFVYGDYKVYDVRK | |
| | MIAKSEQEIGKATAKYFFYS | |
| | NIMNFFKTEITLANGEIRKR | |
| | PLIETNGETGEIVWDKGRDF | |
| | ATVRKVLSMPQVNIVKKTEV | |
| | QTGGFSKESILPKRNSDKLI | |
| | ARKKDWDPKKYGGFDSPTVA | |
| | YSVLVVAKVEKGKSKKLKSV | |
| | KELLGITIMERSSFEKNPID | |
| | FLEAKGYKEVKKDLIIKLPK | |
| | YSLFELENGRKRMLASAGEL | |
| | QKGNELALPSKYVNFLYLAS | |
| | HYEKLKGSPEDNEQKQLFVE | |
| | QHKHYLDEIIEQISEFSKRV | |
| | ILADANLDKVLSAYNKHRDK | |
| | PIREQAENIIHLFTLTNLGA | |
| | PAAFKYFDTTIDRKRYTSTK | |
| | EVLDATLIHQSITGLYETRI | |
| | DLSQLGGD | |

The base editors described herein may include any of the above SpCas9 sequences, or any variant there of having at least 80%, at least 85%, at least 90%, at least 95%, or at least 99% sequence identity thereto.

### Wild Type Cas9 Orthologs

In other embodiments, the Cas9 protein can be a wild type Cas9 ortholog from another bacterial species. For example, the following Cas9 orthologs can be used in connection with the base editor constructs described in this disclosure. In addition, any variant Cas9 orthologs having at least 80%, at least 85%, at least 90%, at least 95%, or at least 99% sequence identity to any of the below orthologs may also be used with the disclosed base editors.

| Description | Sequence |
|---|---|
| LfCas9 | 1 MKEYHIGLDI GTSSIGWAVT DSQFKLMRIK GKTAIGVRLF EEGKTAAERR TFRTTRRRLK |
| Lactobacillus | 61 RRKWRLHYLD EIFAPHLQEV DENFLRRLKQ SNIHPEDPTK NQAFIGKLLF PDLLKKNERG |
| fermentum | 121 YPTLIKMRDE LPVEQRAHYP VMNIYKLREA MINEDRQFDL REVYLAVHHI VKYRGHFLNN |
| wild type | 181 ASVDKFKVGR IDFDKSFNVL NEAYEELQNG EGSFTIEPSK VEKIGQLLLD TKMRKLDRQK |
| GenBank: | 241 AVAKLLEVKV ADKEETKRNK QIATAMSKLV LGYKADFATV AMANGNEWKI DLSSETSEDE |
| SNX31424.1 1 | 301 IEKFREELSD AQNDILTEIT SLFSQIMLNE IVPNGMSISE SMMDRYWTHE RQLAEVKEYL |
| | 361 ATQPASARKE FDQVYNKYIG QAPKERGFDL EKGLKKILSK KENWKEIDEL LKAGDFLPKQ |
| | 421 RTSANGVIPH QMHQQELDRI IEKQAKYYPW LATENPATGE RDRHQAKYEL DQLVSFRIPY |
| | 481 YVGPLVTPEV QKATSGAKFA WAKRKEDGEI TPWNLWDKID RAESAEAFIK RMTVKDTYLL |
| | 541 NEDVLPANSL LYQKYNVLNE LNNVRVNGRR LSVGIKQDIY TELFKKKKTV KASDVASLVM |
| | 601 AKTRGVNKPS VEGLSDPKKF NSNLATYLDL KSIVGDKVDD NRYQTDLENI IEWRSVFEDG |
| | 661 EIFADKLTEV EWLTDEQRSA LVKKRYKGWG RLSKKLLTGI VDENGQRIID LMWNTDQNFK |
| | 721 EIVDQPVFKE QIDQLNQKAI TNDGMTLRER VESVLDDAYT SPQNKKAIWQ VVRVVEDIVK |
| | 781 AVGNAPKSIS IEFARNEGNK GEITRSRRTQ LQKLFEDQAH ELVKDTSLTE ELEKAPDLSD |
| | 841 RYYFYFTQGG KDMYTGDPIN FDEISTKYDI DHILPQSFVK DNSLDNRVLT SRKENNKKSD |
| | 901 QVPAKLYAAK MKPYWNQLLK QGLITQRKFE NLTKDVDQNI KYRSLGFVKR QLVETRQVIK |
| | 961 LTANILGSMY QEAGTEIIET RAGLTKQLRE EFDLPKVREV NDYHHAVDAY LTTFAGQYLN |
| | 1021 RRYPKLRSFF VYGEYMKFKH GSDLKLRNFN FFHELMEGDK SQGKVVDQQT GELITTRDEV |
| | 1081 AKSFDRLLNM KYMLVSKEVH DRSDQLYGAT IVTAKESGKL TSPIEIKKNR LVDLYGAYTN |
| | 1141 GTSAFMTIIK FTGNKPKYKV IGIPTTSAAS LKRAGKPGSE SYNQELHRII KSNPKVKKGF |
| | 1201 EIVVPHVSYG QLIVDGDCKF TLASPTVQHP ATQLVLSKKS LETISSGYKI LKDKPAIANE |
| | 1261 RLIRVFDEVV GQMNRYFTIF DQRSNRQKVA DARDKFLSLP TESKYEGAKK VQVGKTEVIT |
| | 1321 NLLMGLHANA TQGDLKVLGL ATFGFFQSTT GLSLSEDTMI VYQSPTGLFE RRICLKDI |
| | (SEQ ID NO: 8) |
| SaCas9 | MDKKYSIGLD IGTNSVGWAV ITDEYKVPSK KFKVLGNTDR HSIKKNLIGA LLFDSGETAE |
| Staphylococcus | ATRLKRTARR RYTRRKNRIC YLQEIFSNEM AKVDDSFFHR LEESFLVEED KKHERHPIFG |
| aureus wild | NIVDEVAYHE KYPTIYHLRK KLVDSTDKAD LRLIYLALAH MIKFRGHFLI EGDLNPDNSD |
| type | VDKLFIQLVQ TYNQLFEENP INASGVDAKA ILSARLSKSR RLENLIAQLP GEKKNGLFGN |
| GenBank: | LIALSLGLTP NFKSNFDLAE DAKLQLSKDT YDDDLDNLLA QIGDQYADLF LAAKNLSDAI |
| AYD60528.1 | LLSDILRVNT EITKAPLSAS MIKRYDEHHQ DLTLLKALVR QQLPEKYKEI FFDQSKNGYA |
| | GYIDGGASQE EFYKFIKPIL EKMDGTEELL VKLNREDLLR KQRTFDNGSI PHQIHLGELH |
| | AILRRQEDFY PFLKDNREKI EKILTFRIPY YVGPLARGNS RFAWMTRKSE ETITPWNFEE |
| | VVDKGASAQS FIERMTNFDK NLPNEKVLPK HSLLYEYFTV YNELTKVKYV TEGMRKPAFL |
| | SGEQKKAIVD LLFKTNRKVT VKQLKEDYFK KIECFDSVEI SGVEDRFNAS LGTYHDLLKI |
| | IKDKDFLDNE ENEDILEDIV LTLTLFEDRE MIEERLKTYA HLFDDKVMKQ LKRRRYTGWG |
| | RLSRKLINGI RDKQSGKTIL DFLKSDGFAN RNFMQLIHDD SLTFKEDIQK AQVSGQGDSL |
| | HEHIANLAGS PAIKKGILQT VKVVDELVKV MGRHKPENIV IEMARENQTT QKGQKNSRER |
| | MKRIEEGIKE LGSQILKEHP VENTQLQNEK LYLYYLQNGR DMYVDQELDI NRLSDYDVDH |
| | IVPQSFLKDD SIDNKVLTRS DKNRGKSDNV PSEEVVKKMK NYWRQLLNAK LITQRKFDNL |
| | TKAERGGLSE LDKAGFIKRQ LVETRQITKH VAQILDSRMN TKYDENDKLI REVKVITLKS |
| | KLVSDFRKDF QFYKVREINN YHHAHDAYLN AVVGTALIKK YPKLESEFVY GDYKVYDVRK |
| | MIAKSEQEIG KATAKYFFYS NIMNFFKTEI TLANGEIRKR PLIETNGETG EIVWDKGRDF |
| | ATVRKVLSMP QVNIVKKTEV QTGGFSKESI LPKRNSDKLI ARKKDWDPKK YGGFDSPTVA |
| | YSVLVVAKVE KGKSKKLKSV KELLGITIME RSSFEKNPID FLEAKGYKEV KKDLIIKLPK |
| | YSLFELENGR KRMLASAGEL QKGNELALPS KYVNFLYLAS HYEKLKGSPE DNEQKQLFVE |
| | QHKHYLDEII EQISEFSKRV ILADANLDKV LSAYNKHRDK PIREQAENII HLFTLTNLGA |
| | PAAFKYFDTT IDRKRYTSTK EVLDATLIHQ SITGLYETRI DLSQLGGD |
| | (SEQ ID NO: 9) |
| SaCas9 | MGKRNYILGLDIGITSVGYGIIDYETRDVIDAGVRLFKEANVENNEGRRSKRGARRLKRRRRHRIQRVKKLL |
| Staphylococcus | FDYNLLTDHSELSGINPYEARVKGLSQKLSEEEFSAALLHLAKRRGVHNVNEVEEDTGNELSTKEQISRNSK |
| aureus | ALEEKYVAELQLERLKKDGEVRGSINRFKTSDYVKEAKQLLKVQKAYHQLDQSFIDTYIDLLETRRTYYEGP |
| | GEGSPFGWKDIKEWYEMLMGHCTYFPEELRSVKYAYNADLYNALNDLNNLVITRDENEKLEYYEKFQIIENV |
| | FKQKKKPTLKQIAKEILVNEEDIKGYRVTSTGKPEFTNLKVYHDIKDITARKEIIENAELLDQIAKILTIYQ |
| | SSEDIQEELTNLNSELTQEEIEQISNLKGYTGTHNLSLKAINLILDELWHTNDNQIAIFNRLKLVPKKVDLS |
| | QQKEIPTTLVDDFILSPVVKRSFIQSIKVINAIIKKYGLPNDIIIELAREKNSKDAQKMINEMQKRNRQTNE |
| | RIEEIIRTTGKENAKYLIEKIKLHDMQEGKCLYSLEAIPLEDLLNNPFNYEVDHIIPRSVSFDNSFNNKVLV |
| | KQEENSKKGNRTPFQYLSSSDSKISYETFKKHILNLAKGKGRISKTKKEYLLEERDINRFSVQKDFINRNLV |
| | DTRYATRGLMNLLRSYFRVNNLDVKVKSINGGFTSFLRRKWKFKKERNKGYKHHAEDALIIANADFIFKEWK |
| | KLDKAKKVMENQMFEEKQAESMPEIETEQEYKEIFITPHQIKHIKDFKDYKYSHRVDKKPNRKLINDTLYST |
| | RKDDKGNTLIVNNLNGLYDKDNDKLKKLINKSPEKLLMYHHDPQTYQKLKLIMEQYGDEKNPLYKYYEETGN |
| | YLTKYSKKDNGPVIKKIKYYGNKLNAHLDITDDYPNSRNKVVKLSLKPYRFDVYLDNGVYKFVTVKNLDVIK |
| | KENYYEVNSKCYEEAKKLKKISNQAEFIASFYKNDLIKINGELYRVIGVNNDLLNRIEVNMIDITYREYLEN |
| | MNDKRPPHIIKTIASKTQSIKKYSTDILGNLYEVKSKKHPQIIKK |
| | (SEQ ID NO: 11) |
| StCas9 | 1 MLFNKCIIIS INLDFSNKEK CMTKPYSIGL DIGTNSVGWA VITDNYKVPS KKMKVLGNTS |
| Streptococcus | 61 KKYIKKNLLG VLLFDSGITA EGRRLKRTAR RRYTRRRNRI LYLQEIFSTE MATLDDAFFQ |
| thermophilus | 121 RLDDSFLVPD DKRDSKYPIF GNLVEEKVYH DEFPTIYHLR KYLADSTKKA DLRLVYLALA |
| UniProtKB/ | 181 HMIKYRGHFL IEGEFNSKNN DIQKNFQDFL DTYNAIFESD LSLENSKQLE EIVKDKISKL |
| Swiss-Prot: | 241 EKKDRILKLF PGEKNSGIFS EFLKLIVGNQ ADFRKCFNLD EKASLHFSKE SYDEDLETLL |
| G3ECR1.2 | 301 GYIGDDYSDV FLKAKKLYDA ILLSGFLTVT DNETEAPLSS AMIKRYNEHK EDLALLKEYI |
| Wild type | 361 RNISLKTYNE VFKDDTKNGY AGYIDGKTNQ EDFYVYLKNL LAEFEGADYF LEKIDREDFL |
| | 421 RKQRTFDNGS IPYQIHLQEM RAILDKQAKF YPFLAKNKER IEKILTFRIP YYVGPLARGN |
| | 481 SDFAWSIRKR NEKITPWNFE DVIDKESSAE AFINRMTSFD LYLPEEKVLP KHSLLYETFN |
| | 541 VYNELTKVRF IAESMRDYQF LDSKQKKDIV RLYFKDKRKV TDKDIIEYLH AIYGYDGIEL |
| | 601 KGIEKQFNSS LSTYHDLLNI INDKEFLDDS SNEAIIEEII HTLTIFEDRE MIKQRLSKFE |
| | 661 NIFDKSVLKK LSRRHYTGWG KLSAKLINGI RDEKSGNTIL DYLIDDGISN RNFMQLIHDD |
| | 721 ALSFKKKIQK AQIIGDEDKG NIKEVVKSLP GSPAIKKGIL QSIKIVDELV KVMGGRKPES |
| | 781 IVVEMARENQ YTNQGKSNSQ QRLKRLEKSL KELGSKILKE NIPAKLSKID NNALQNDRLY |
| | 841 LYYLQNGKDM YTGDDLDIDR LSNYDIDHII PQAFLKDNSI DNKVLVSSAS NRGKSDDFPS |
| | 901 LEVVKKRKTF WYQLLKSKLI SQRKFDNLTK AERGGLLPED KAGFIQRQLV ETRQITKHVA |
| | 961 RLLDEKFNNK KDENNRAVRT VKIITLKSTL VSQFRKDFEL YKVREINDFH HAHDAYLNAV |
| | 1021 IASALLKKYP KLEPEFVYGD YPKYNSFRER KSATEKVYFY SNIMNIFKKS ISLADGRVIE |
| | 1081 RPLIEVNEET GESVWNKESD LATVRRVLSY PQVNVVKKVE EQNHGLDRGK PKGLFNANLS |
| | 1141 SKPKPNSNEN LVGAKEYLDP KKYGGYAGIS NSFAVLVKGT IEKGAKKKIT NVLEFQGISI |
| | 1201 LDRINYRKDK LNFLLEKGYK DIELIIELPK YSLFELSDGS RRMLASILST NNKRGEIHKG |
| | 1261 NQIFLSQKFV KLLYHAKRIS NTINENHRKY VENHKKEFEE LFYYILEFNE NYVGAKKNGK |
| | 1321 LLNSAFQSWQ NHSIDELCSS FIGPTGSERK GLFELTSRGS AADFEFLGVK IPRYRDYTPS |
| | 1381 SLLKDATLIH QSVTGLYETR IDLAKLGEG |
| | (SEQ ID NO: 12) |
| LcCas9 | 1 MKIKNYNLAL TPSTSAVGHV EVDDDLNILE PVHHQKAIGV AKFGEGETAE ARRLARSARR |
| Lactobacillus | 61 TTKRRANRIN HYFNEIMKPE IDKVDPLMFD RIKQAGLSPL DERKEFRTVI FDRPNIASYY |
| crispatus | 121 HNQFPTIWHL QKYLMITDEK ADIRLIYWAL HSLLKHRGHF FNTTPMSQFK PGKLNLKDDM |
| NCBI Reference | 181 LALDDYNDLE GLSFAVANSP EIEKVIKDRS MHKKEKIAEL KKLIVNDVPD KDLAKRNNKI |
| Sequence: | 241 ITQIVNAIMG NSFHLNFIFD MDLDKLTSKA WSFKLDDPEL DTKFDAISGS MTDNQIGIFE |
| WP_133478044. | 301 TLQKIYSAIS LLDILNGSSN VVDAKNALYD KHKRDLNLYF KFLNTLPDEI AKTLKAGYTL |
| 1 | 361 YIGNRKKDLL AARKLLKVNV AKNFSQDDFY KLINKELKSI DKQGLQTRFS EKVGELVAQN |
| Wild type | 421 NFLPVQRSSD NVFIPYQLNA ITFNKILENQ GKYYDFLVKP NPAKKDRKNA PYELSQLMQF |
| | 481 TIPYYVGPLV TPEEQVKSGI PKTSRFAWMV RKDNGAITPW NFYDKVDIEA TADKFIKRSI |
| | 541 AKDSYLLSEL VLPKHSLLYE KYEVFNELSN VSLDGKKLSG GVKQILFNEV FKKTNKVNTS |
| | 601 RILKALAKHN IPGSKITGLS NPEEFTSSLQ TYNAWKKYFP NQIDNFAYQQ DLEKMIEWST |
| | 661 VFEDHKILAK KLDEIEWLDD DQKKFVANTR LRGWGRLSKR LLTGLKDNYG KSIMQRLETT |
| | 721 KANFQQIVYK PEFREQIDKI SQAAAKNQSL EDILANSYTS PSNRKAIRKT MSVVDEYIKL |
| | 781 NHGKEPDKIF LMFQRSEQEK GKQTEARSKQ LNRILSQLKA DKSANKLFSK QLADEFSNAI |
| | 841 KKSKYKLNDK QYFYFQQLGR DALTGEVIDY DELYKYTVLH IIPRSKLTDD SQNNKVLTKY |
| | 901 KIVDGSVALK FGNSYSDALG MPIKAFWTEL NRLKLIPKGK LLNLTTDFST LNKYQRDGYI |
| | 961 ARQLVETQQI VKLLATIMQS RFKHTKIIEV RNSQVANIRY QFDYFRIKNL NEYYRGFDAY |
| | 1021 LAAVVGTYLY KVYPKARRLF VYGQYLKPKK TNQENQDMHL DSEKKSQGFN FLWNLLYGKQ |
| | 1081 DQIFVNGTDV IAFNRKDLIT KMNTVYNYKS QKISLAIDYH NGAMFKATLF PRNDRDTAKT |
| | 1141 RKLIPKKKDY DTDIYGGYTS NVDGYMLLAE IIKRDGNKQY GFYGVPSRLV SELDTLKKTR |
| | 1201 YTEYEEKLKE IIKPELGVDL KKIKKIKILK NKVPFNQVII DKGSKFFITS TSYRWNYRQL |
| | 1261 ILSAESQQTL MDLVVDPDFS NHKARKDARK NADERLIKVY EEILYQVKNY MPMFVELHRC |
| | 1321 YEKLVDAQKT FKSLKISDKA MVLNQILILL HSNATSPVLE KLGYHTRFTL GKKHNLISEN |
| | 1381 AVLVTQSITG LKENHVSIKQ ML |
| | (SEQ ID NO: 13) |
| PdCas9 | 1 MTNEKYSIGL DIGTSSIGFA VVNDNNRVIR VKGKNAIGVR LFDEGKAAAD RRSFRTTRRS |
| Pedicoccus | 61 FRTTRRRLSR RRWRLKLLRE IFDAYITPVD EAFFIRLKES NLSPKDSKKQ YSGDILFNDR |
| damnosus | 121 SDKDFYEKYP TIYHLRNALM TEHRKFDVRE IYLAIHHIMK FRGHFLNATP ANNFKVGRLN |
| NCBI Reference | 181 LEEKFEELND IYQRVFPDES IEFRTDNLEQ IKEVLLDNKR SRADRQRTLV SDIYQSSEDK |
| Sequence: | 241 DIEKRNKAVA TEILKASLGN KAKLNVITNV EVDKEAAKEW SITFDSESID DDLAKIEGQM |
| WP_062913273. | 301 TDDGHEIIEV LRSLYSGITL SAIVPENHTL SQSMVAKYDL HKDHLKLFKK LINGMTDTKK |
| 1 | 361 AKNLRAAYDG YIDGVKGKVL PQEDFYKQVQ VNLDDSAEAN EIQTYIDQDI FMPKQRTKAN |
| Wild type | 421 GSIPHQLQQQ ELDQIIENQK AYYPWLAELN PNPDKKRQQL AKYKLDELVT FRVPYYVGPM |
| | 481 ITAKDQKNQS GAEFAWMIRK EPGNITPWNF DQKVDRMATA NQFIKRMTTT DTYLLGEDVL |
| | 541 PAQSLLYQKF EVLNELNKIR IDHKPISIEQ KQQIFNDLFK QFKNVTIKHL QDYLVSQGQY |
| | 601 SKRPLIEGLA DEKRFNSSLS TYSDLCGIFG AKLVEENDRQ EDLEKIIEWS TIFEDKKIYR |
| | 661 AKLNDLTWLT DDQKEKLATK RYQGWGRLSR KLLVGLKNSE HRNIMDILWI TNENFMQIQA |
| | 721 EPDFAKLVTD ANKGMLEKTD SQDVINDLYT SPQNKKAIRQ ILLVVHDIQN AMHGQAPAKI |
| | 781 HVEFARGEER NPRRSVQRQR QVEAAYEKVS NELVSAKVRQ EFKEAINNKR DFKDRLFLYF |
| | 841 MQGGIDIYTG KQLNIDQLSS YQIDHILPQA FVKDDSLTNR VLTNENQVKA DSVPIDIFGK |
| | 901 KMLSVWGRMK DQGLISKGKY RNLTMNPENI SAHTENGFIN RQLVETRQVI KLAVNILADE |
| | 961 YGDSTQIISV KADLSHQMRE DFELLKNRDV NDYHHAFDAY LAAFIGNYLL KRYPKLESYF |
| | 1021 VYGDFKKFTQ KETKMRRFNF IYDLKHCDQV VNKETGEILW TKDEDIKYIR HLFAYKKILV |
| | 1081 SHEVREKRGA LYNQTIYKAK DDKGSGQESK KLIRIKDDKE TKIYGGYSGK SLAYMTIVQI |
| | 1141 TKKNKVSYRV IGIPTLALAR LNKLENDSTE NNGELYKIIK PQFTHYKVDK KNGEIIETTD |
| | 1201 DFKIVVSKVR FQQLIDDAGQ FFMLASDTYK NNAQQLVISN NALKAINNTN ITDCPRDDLE |
| | 1261 RLDNLRLDSA FDEIVKKMDK YFSAYDANNF REKIRNSNLI FYQLPVEDQW ENNKITELGK |
| | 1321 RTVLTRILQG LHANATTTDM SIFKIKTPFG QLRQRSGISL SENAQLIYQS PTGLFERRVQ |
| | 1381 LNKIK |
| | (SEQ ID NO: 14) |
| FnCas9 | 1 MKKQKFSDYY LGFDIGTNSV GWCVTDLDYN VLRFNKKDMW GSRLFEEAKT AAERRVQRNS |
| Fusobacterium | 61 RRRLKRRKWR LNLLEEIFSN EILKIDSNFF RRLKESSLWL EDKSSKEKFT LFNDDNYKDY |
| nucleatum | 121 DFYKQYPTIF HLRNELIKNP EKKDIRLVYL AIHSIFKSRG HFLFEGQNLK EIKNFETLYN |
| NCBI Reference | 181 NLIAFLEDNG INKIIDKNNI EKLEKIVCDS KKGLKDKEKE FKEIFNSDKQ LVAIFKLSVG |
| Sequence: | 241 SSVSLNDLFD TDEYKKGEVE KEKISFREQI YEDDKPIYYS ILGEKIELLD IAKTFYDFMV |
| WP_060798984. | 301 LNNILADSQY ISEAKVKLYE EHKKDLKNLK YIIRKYNKGN YDKLFKDKNE NNYSAYIGLN |
| 1 | 361 KEKSKKEVIE KSRLKIDDLI KNIKGYLPKV EEIEEKDKAI FNKILNKIEL KTILPKQRIS |
| | 421 DNGTLPYQIH EAELEKILEN QSKYYDFLNY EENGIITKDK LLMTFKFRIP YYVGPLNSYH |
| | 481 KDKGGNSWIV RKEEGKILPW NFEQKVDIEK SAEEFIKRMT NKCTYLNGED VIPKDTFLYS |
| | 541 EYVILNELNK VQVNDEFLNE ENKRKIIDEL FKENKKVSEK KFKEYLLVKQ IVDGTIELKG |
| | 601 VKDSFNSNYI SYIRFKDIFG EKLNLDIYKE ISEKSILWKC LYGDDKKIFE KKIKNEYGDI |
| | 661 LTKDEIKKIN TFKFNNWGRL SEKLLTGIEF INLETGECYS SVMDALRRTN YNLMELLSSK |
| | 721 FTLQESINNE NKEMNEASYR DLIEESYVSP SLKRAIFQTL KIYEEIRKIT GRVPKKVFIE |
| | 781 MARGGDESMK NKKIPARQEQ LKKLYDSCGN DIANFSIDIK EMKNSLISYD NNSLRQKKLY |
| | 841 LYYLQFGKCM YTGREIDLDR LLQNNDTYDI DHIYPRSKVI KDDSFDNLVL VLKNENAEKS |
| | 901 NEYPVKKEIQ EKMKSFWRFL KEKNFISDEK YKRLTGKDDF ELRGFMARQL VNVRQTTKEV |
| | 961 GKILQQIEPE IKIVYSKAEI ASSFREMFDF IKVRELNDTH HAKDAYLNIV AGNVYNTKFT |
| | 1021 EKPYRYLQEI KENYDVKKIY NYDIKNAWDK ENSLEIVKKN MEKNTVNITR FIKEKKGQLF |
| | 1081 DLNPIKKGET SNEIISIKPK VYNGKDDKLN EKYGYYKSLN PAYFLYVEHK EKNKRIKSFE |
| | 1141 RVNLVDVNNI KDEKSLVKYL IENKKLVEPR VIKKVYKRQV ILINDYPYSI VTLDSNKLMD |
| | 1201 FENLKPLFLE NKYEKILKNV IKFLEDNQGK SEENYKFIYL KKKDRYEKNE TLESVKDRYN |
| | 1261 LEFNEMYDKF LEKLDSKDYK NYMNNKKYQE LLDVKEKFIK LNLFDKAFTL KSFLDLFNRK |
| | 1321 TMADFSKVGL TKYLGKIQKI SSNVLSKNEL YLLEESVTGL FVKKIKL |
| | (SEQ ID NO: 82) |
| EcCas9 | 61 RRKQRIQILQ ELLGEEVLKT DPGFFHRMKE SRYVVEDKRT LDGKQVELPY ALFVDKDYTD |
| Enterococcus | 121 KEYYKQFPTI NHLIVYLMTT SDTPDIRLVY LALHYYMKNR GNFLHSGDIN NVKDINDILE |
| cecorum | 181 QLDNVLETFL DGWNLKLKSY VEDIKNIYNR DLGRGERKKA FVNTLGAKTK AEKAFCSLIS |
| NCBI Reference | 241 GGSTNLAELF DDSSLKEIET PKIEFASSSL EDKIDGIQEA LEDRFAVIEA AKRLYDWKTL |
| Sequence: | 301 TDILGDSSSL AEARVNSYQM HHEQLLELKS LVKEYLDRKV FQEVFVSLNV ANNYPAYIGH |
| WP_047338501. | 361 TKINGKKKEL EVKRTKRNDF YSYVKKQVIE PIKKKVSDEA VLTKLSEIES LIEVDKYLPL |
| 1 | 421 QVNSDNGVIP YQVKLNELTR IFDNLENRIP VLRENRDKII KTFKFRIPYY VGSLNGVVKN |
| Wild type | 481 GKCTNWMVRK EEGKIYPWNF EDKVDLEASA EQFIRRMTNK CTYLVNEDVL PKYSLLYSKY |
| | 541 LVLSELNNLR IDGRPLDVKI KQDIYENVFK KNRKVTLKKI KKYLLKEGII TDDDELSGLA |
| | 601 DDVKSSLTAY RDFKEKLGHL DLSEAQMENI ILNITLFGDD KKLLKKRLAA LYPFIDDKSL |
| | 661 NRIATLNYRD WGRLSERFLS GITSVDQETG ELRTIIQCMY ETQANLMQLL AEPYHFVEAI |
| | 721 EKENPKVDLE SISYRIVNDL YVSPAVKRQI WQTLLVIKDI KQVMKHDPER IFIEMAREKQ |
| | 781 ESKKTKSRKQ VLSEVYKKAK EYEHLFEKLN SLTEEQLRSK KIYLYFTQLG KCMYSGEPID |
| | 841 FENLVSANSN YDIDHIYPQS KTIDDSFNNI VLVKKSLNAY KSNHYPIDKN IRDNEKVKTL |
| | 901 WNTLVSKGLI TKEKYERLIR STPFSDEELA GFIARQLVET RQSTKAVAEI LSNWFPESEI |
| | 961 VYSKAKNVSN FRQDFEILKV RELNDCHHAH DAYLNIVVGN AYHTKFTNSP YRFIKNKANQ |
| | 1021 EYNLRKLLQK VNKIESNGVV AWVGQSENNP GTIATVKKVI RRNTVLISRM VKEVDGQLFD |
| | 1081 LTLMKKGKGQ VPIKSSDERL TDISKYGGYN KATGAYFTFV KSKKRGKVVR SFEYVPLHLS |
| | 1141 KQFENNNELL KEYIEKDRGL TDVEILIPKV LINSLFRYNG SLVRITGRGD TRLLLVHEQP |
| | 1201 LYVSNSFVQQ LKSVSSYKLK KSENDNAKLT KTATEKLSNI DELYDGLLRK LDLPIYSYWF |
| | 1261 SSIKEYLVES RTKYIKLSIE EKALVIFEIL HLFQSDAQVP NLKILGLSTK PSRIRIQKNL |
| | 1321 KDTDKMSIIH QSPSGIFEHE IELTSL (SEQ ID NO: 15) |
| AhCas9 | 1 MQNGFLGITV SSEQVGWAVT NPKYELERAS RKDLWGVRLF DKAETAEDRR MFRTNRRLNQ |
| Anaerostipes | 61 RKKNRIHYLR DIFHEEVNQK DPNFFQQLDE SNFCEDDRTV EFNFDTNLYK NQFPTVYHLR |
| hadrus | 121 KYLMETKDKP DIRLVYLAFS KFMKNRGHFL YKGNLGEVMD FENSMKGFCE SLEKFNIDFP |
| NCBI Reference | 181 TLSDEQVKEV RDILCDHKIA KTVKKKNIIT ITKVKSKTAK AWIGLFCGCS VPVKVLFQDI |
| Sequence: | 241 DEEIVTDPEK ISFEDASYDD YIANIEKGVG IYYEAIVSAK MLFDWSILNE ILGDHQLLSD |
| WP_044924278. | 301 AMIAEYNKHH DDLKRLQKII KGTGSRELYQ DIFINDVSGN YVCYVGHAKT MSSADQKQFY |
| 1 | 361 TFLKNRLKNV NGISSEDAEW IDTEIKNGTL LPKQTKRDNS VIPHQLQLRE FELILDNMQE |
| Wild type | 421 MYPFLKENRE KLLKIFNFVI PYYVGPLKGV VRKGESTNWM VPKKDGVIHP WNFDEMVDKE |
| | 481 ASAECFISRM TGNCSYLFNE KVLPKNSLLY ETFEVLNELN PLKINGEPIS VELKQRIYEQ |
| | 541 LFLTGKKVTK KSLTKYLIKN GYDKDIELSG IDNEFHSNLK SHIDFEDYDN LSDEEVEQII |
| | 601 LRITVFEDKQ LLKDYLNREF VKLSEDERKQ ICSLSYKGWG NLSEMLLNGI TVTDSNGVEV |
| | 661 SVMDMLWNTN LNLMQILSKK YGYKAEIEHY NKEHEKTIYN REDLMDYLNI PPAQRRKVNQ |
| | 721 LITIVKSLKK TYGVPNKIFF KISREHQDDP KRTSSRKEQL KYLYKSLKSE DEKHLMKELD |
| | 781 ELNDHELSND KVYLYFLQKG RCIYSGKKLN LSRLRKSNYQ NDIDYIYPLS AVNDRSMNNK |
| | 841 VLTGIQENRA DKYTYFPVDS EIQKKMKGFW MELVLQGFMT KEKYFRLSRE NDFSKSELVS |
| | 901 FIEREISDNQ QSGRMIASVL QYYFPESKIV FVKEKLISSF KRDFHLISSY GHNHLQAAKD |
| | 961 AYITIVVGNV YHTKFTMDPA IYFKNHKRKD YDLNRLFLEN ISRDGQIAWE SGPYGSIQTV |
| | 1021 RKEYAQNHIA VTKRVVEVKG GLFKQMPLKK GHGEYPLKTN DPRFGNIAQY GGYTNVTGSY |
| | 1081 FVLVESMEKG KKRISLEYVP VYLHERLEDD PGHKLLKEYL VDHRKLNHPK ILLAKVRKNS |
| | 1141 LLKIDGFYYR LNGRSGNALI LTNAVELIMD DWQTKTANKI SGYMKRRAID KKARVYQNEF |
| | 1201 HIQELEQLYD FYLDKLKNGV YKNRKNNQAE LIHNEKEQFM ELKTEDQCVL LTEIKKLFVC |
| | 1261 SPMQADLTLI GGSKHTGMIA MSSNVTKADF AVIAEDPLGL RNKVIYSHKG EK |
| | (SEQ ID NO: 16) |
| KvCas9 | 1 MSQNNNKIYN IGLDIGDASV GWAVVDEHYN LLKRHGKHMW GSRLFTQANT AVERRSSRST |
| Kandleria | 61 RRRYNKRRER IRLLREIMED MVLDVDPTFF IRLANVSFLD QEDKKDYLKE NYHSNYNLFI |
| vitulina | 121 DKDFNDKTYY DKYPTIYHLR KHLCESKEKE DPRLIYLALH HIVKYRGNFL YEGQKFSMDV |
| NCBI Reference | 181 SNIEDKMIDV LRQFNEINLF EYVEDRKKID EVLNVLKEPL SKKHKAEKAF ALFDTTKDNK |
| Sequence: | 241 AAYKELCAAL AGNKFNVTKM LKEAELHDED EKDISFKFSD ATFDDAFVEK QPLLGDCVEF |
| WP_031589969. | 301 IDLLHDIYSW VELQNILGSA HTSEPSISAA MIQRYEDHKN DLKLLKDVIR KYLPKKYFEV |
| 1 | 361 FRDEKSKKNN YCNYINHPSK TPVDEFYKYI KKLIEKIDDP DVKTILNKIE LESFMLKQNS |
| Wild type | 421 RTNGAVPYQM QLDELNKILE NQSVYYSDLK DNEDKIRSIL TFRIPYYFGP LNITKDRQFD |
| | 481 WIIKKEGKEN ERILPWNANE IVDVDKTADE FIKRMRNFCT YFPDEPVMAK NSLTVSKYEV |
| | 541 LNEINKLRIN DHLIKRDMKD KMLHTLFMDH KSISANAMKK WLVKNQYFSN TDDIKIEGFQ |
| | 601 KENACSTSLT PWIDFTKIFG KINESNYDFI EKIIYDVTVF EDKKILRRRL KKEYDLDEEK |
| | 661 IKKILKLKYS GWSRLSKKLL SGIKTKYKDS TRTPETVLEV MERTNMNLMQ VINDEKLGFK |
| | 721 KTIDDANSTS VSGKFSYAEV QELAGSPAIK RGIWQALLIV DEIKKIMKHE PAHVYIEFAR |
| | 781 NEDEKERKDS FVNQMLKLYK DYDFEDETEK EANKHLKGED AKSKIRSERL KLYYTQMGKC |
| | 841 MYTGKSLDID RLDTYQVDHI VPQSLLKDDS IDNKVLVLSS ENQRKLDDLV IPSSIRNKMY |
| | 901 GFWEKLFNNK IISPKKFYSL IKTEFNEKDQ ERFINRQIVE TRQITKHVAQ IIDNHYENTK |
| | 961 VVTVRADLSH QFRERYHIYK NRDINDFHHA HDAYIATILG TYIGHRFESL DAKYIYGEYK |
| | 1021 RIFRNQKNKG KEMKKNNDGF ILNSMRNIYA DKDTGEIVWD PNYIDRIKKC FYYKDCFVTK |
| | 1081 KLEENNGTFF NVTVLPNDTN SDKDNTLATV PVNKYRSNVN KYGGFSGVNS FIVAIKGKKK |
| | 1141 KGKKVIEVNK LTGIPLMYKN ADEEIKINYL KQAEDLEEVQ IGKEILKNQL IEKDGGLYYI |
| | 1201 VAPTEIINAK QLILNESQTK LVCEIYKAMK YKNYDNLDSE KIIDLYRLLI NKMELYYPEY |
| | 1261 RKQLVKKFED RYEQLKVISI EEKCNIIKQI LATLHCNSSI GKIMYSDFKI STTIGRLNGR |
| | 1321 TISLDDISFI AESPTGMYSK KYKL (SEQ ID NO: 17) |
| EfCas9 | 1 MRLFEEGHTA EDRRLKRTAR RRISRRRNRL RYLQAFFEEA MTDLDENFFA RLQESFLVPE |
| Enterococcus | 61 DKKWHRHPIF AKLEDEVAYH ETYPTIYHLR KKLADSSEQA DLRLIYLALA HIVKYRGHFL |
| faecalis | 121 IEGKLSTENT SVKDQFQQFM VIYNQTFVNG ESRLVSAPLP ESVLIEEELT EKASRTKKSE |
| NCBI | 181 KVLQQFPQEK ANGLFGQFLK LMVGNKADFK KVFGLEEEAK ITYASESYEE DLEGILAKVG |
| Reference | 241 DEYSDVFLAA KNVYDAVELS TILADSDKKS HAKLSSSMIV RFTEHQEDLK KFKRFIRENC |
| Sequence: | 301 PDEYDNLFKN EQKDGYAGYI AHAGKVSQLK FYQYVKKIIQ DIAGAEYFLE KIAQENFLRK |
| WP_016631044. | 361 QRTFDNGVIP HQIHLAELQA IIHRQAAYYP FLKENQEKIE QLVTFRIPYY VGPLSKGDAS |
| 1 | 421 TFAWLKRQSE EPIRPWNLQE TVDLDQSATA FIERMTNFDT YLPSEKVLPK HSLLYEKFMV |
| Wild type | 481 FNELTKISYT DDRGIKANFS GKEKEKIFDY LFKTRRKVKK KDIIQFYRNE YNTEIVTLSG |
| | 541 LEEDQFNASF STYQDLLKCG LTRAELDHPD NAEKLEDIIK ILTIFEDRQR IRTQLSTFKG |
| | 601 QFSAEVLKKL ERKHYTGWGR LSKKLINGIY DKESGKTILD YLVKDDGVSK HYNRNFMQLI |
| | 661 NDSQLSFKNA IQKAQSSEHE ETLSETVNEL AGSPAIKKGI YQSLKIVDEL VAIMGYAPKR |
| | 721 IVVEMARENQ TTSTGKRRSI QRLKIVEKAM AEIGSNLLKE QPTTNEQLRD TRLFLYYMQN |
| | 781 GKDMYTGDEL SLHRLSHYDI DHIIPQSFMK DDSLDNLVLV GSTENRGKSD DVPSKEVVKD |
| | 841 MKAYWEKLYA AGLISQRKFQ RLTKGEQGGL TLEDKAHFIQ RQLVETRQIT KNVAGILDQR |
| | 901 YNAKSKEKKV QIITLKASLT SQFRSIFGLY KVREVNDYHH GQDAYLNCVV ATTLLKVYPN |
| | 961 LAPEFVYGEY PKFQTFKENK ATAKAIIYTN LLRFFTEDEP RFTKDGEILW SNSYLKTIKK |
| | 1021 ELNYHQMNIV KKVEVQKGGF SKESIKPKGP SNKLIPVKNG LDPQKYGGFD SPVVAYTVLF |
| | 1081 THEKGKKPLI KQEILGITIM EKTRFEQNPI LFLEEKGFLR PRVLMKLPKY TLYEFPEGRR |
| | 1141 RLLASAKEAQ KGNQMVLPEH LLTLLYHAKQ CLLPNQSESL AYVEQHQPEF QEILERVVDF |
| | 1201 AEVHTLAKSK VQQIVKLFEA NQTADVKEIA ASFIQLMQFN AMGAPSTFKF FQKDIERARY |
| | 1261 TSIKEIFDAT IIYQSPTGLY ETRRKVVD (SEQ ID NO: 18) |
| Staphylococcus | KRNYILGLDIGITSVGYGIIDYETRDVIDAGVRLFKEANVENNEGRRSKRGARRLKRRRRHRIQRVKKLLFD |
| aureus Cas9 | YNLLTDHSELSGINPYEARVKGLSQKLSEEEFSAALLHLAKRRGVHNVNEVEEDTGNELSTKEQISRNSKAL |
| | EEKYVAELQLERLKKDGEVRGSINRFKTSDYVKEAKQLLKVQKAYHQLDQSFIDTYIDLLETRRTYYEGPGE |
| | GSPFGWKDIKEWYEMLMGHCTYFPEELRSVKYAYNADLYNALNDLNNLVITRDENEKLEYYEKFQIIENVFK |
| | QKKKPTLKQIAKEILVNEEDIKGYRVTSTGKPEFTNLKVYHDIKDITARKEIIENAELLDQIAKILTIYQSS |
| | EDIQEELTNLNSELTQEEIEQISNLKGYTGTHNLSLKAINLILDELWHTNDNQIAIFNRLKLVPKKVDLSQQ |
| | KEIPTTLVDDFILSPVVKRSFIQSIKVINAIIKKYGLPNDIIIELAREKNSKDAQKMINEMQKRNRQTNERI |
| | EEIIRTTGKENAKYLIEKIKLHDMQEGKCLYSLEAIPLEDLLNNPFNYEVDHIIPRSVSFDNSFNNKVLVKQ |
| | EENSKKGNRTPFQYLSSSDSKISYETFKKHILNLAKGKGRISKTKKEYLLEERDINRFSVQKDFINRNLVDT |
| | RYATRGLMNLLRSYFRVNNLDVKVKSINGGFTSFLRRKWKFKKERNKGYKHHAEDALIIANADFIFKEWKKL |
| | DKAKKVMENQMFEEKQAESMPEIETEQEYKEIFITPHQIKHIKDFKDYKYSHRVDKKPNRELINDTLYSTRK |
| | DDKGNTLIVNNLNGLYDKDNDKLKKLINKSPEKLLMYHHDPQTYQKLKLIMEQYGDEKNPLYKYYEETGNYL |
| | TKYSKKDNGPVIKKIKYYGNKLNAHLDITDDYPNSRNKVVKLSLKPYRFDVYLDNGVYKFVTVKNLDVIKKE |
| | NYYEVNSKCYEEAKKLKKISNQAEFIASFYNNDLIKINGELYRVIGVNNDLLNRIEVNMIDITYREYLENMN |
| | DKRPPRIIKTIASKTQSIKKYSTDILGNLYEVKSKKHPQIIKKG |
| | (SEQ ID NO: 216) |
| ScCas9 | MEKKYSIGLDIGTNSVGWAVITDDYKVPSKKFKVLGNTNRKSIKKNLMGALLFDSGETAEATRLKRTARRRY |
| S. canis | TRRKNRIRYLQEIFANEMAKLDDSFFQRLEESFLVEEDKKNERHPIFGNLADEVAYHRNYPTIYHLRKKLAD |
| 1375 AA | SPEKADLRLIYLALAHIIKFRGHFLIEGKLNAENSDVAKLFYQLIQTYNQLFEESPLDEIEVDAKGILSARL |
| 159.2 kDa | SKSKRLEKLIAVFPNEKKNGLFGNIIALALGLTPNFKSNFDLTEDAKLQLSKDTYDDDLDELLGQIGDQYAD |
| | LFSAAKNLSDAILLSDILRSNSEVTKAPLSASMVKRYDEHHQDLALLKTLVRQQFPEKYAEIFKDDTKNGYA |
| | GYVGIGIKHRKRTTKLATQEEFYKFIKPILEKMDGAEELLAKLNRDDLLRKQRTFDNGSIPHQIHLKELHAI |
| | LRRQEEFYPFLKENREKIEKILTFRIPYYVGPLARGNSRFAWLTRKSEEAITPWNFEEVVDKGASAQSFIER |
| | MTNFDEQLPNKKVLPKHSLLYEYFTVYNELTKVKYVTERMRKPEFLSGEQKKAIVDLLFKTNRKVTVKQLKE |
| | DYFKKIECFDSVEIIGVEDRFNASLGTYHDLLKIIKDKDFLDNEENEDILEDIVLTLTLFEDREMIEERLKT |
| | YAHLFDDKVMKQLKRRHYTGWGRLSRKMINGIRDKQSGKTILDFLKSDGFSNRNFMQLIHDDSLTFKEEIEK |
| | AQVSGQGDSLHEQIADLAGSPAIKKGILQTVKIVDELVKVMGHKPENIVIEMARENQTTTKGLQQSRERKKR |
| | IEEGIKELESQILKENPVENTQLQNEKLYLYYLQNGRDMYVDQELDINRLSDYDVDHIVPQSFIKDDSIDNK |
| | VLTRSVENRGKSDNVPSEEVVKKMKNYWRQLLNAKLITQRKFDNLTKAERGGLSEADKAGFIKRQLVETRQI |
| | TKHVARILDSRMNTKRDKNDKPIREVKVITLKSKLVSDFRKDFQLYKVRDINNYHHAHDAYLNAVVGTALIK |
| | KYPKLESEFVYGDYKVYDVRKMIAKSEQEIGKATAKRFFYSNIMNFFKTEVKLANGEIRKRPLIETNGETGE |
| | VVWNKEKDFATVRKVLAMPQVNIVKKTEVQTGGFSKESILSKRESAKLIPRKKGWDTRKYGGFGSPTVAYSI |
| | LVVAKVEKGKAKKLKSVKVLVGITIMEKGSYEKDPIGFLEAKGYKDIKKELIFKLPKYSLFELENGRRRMLA |
| | SATELQKANELVLPQHLVRLLYYTQNISATTGSNNLGYIEQHREEFKEIFEKIIDFSEKYILKNKVNSNLKS |
| | SFDEQFAVSDSILLSNSFVSLLKYTSFGASGGFTFLDLDVKQGRLRYQTVTEVLDATLIYQSITGLYETRTD |
| | LSQLGGD (SEQ ID NO: 19) |

The base editors described herein may include any of the above Cas9 ortholog sequences, or any variants thereof having at least 80%, at least 85%, at least 90%, at least 95%, or at least 99% sequence identity thereto.

The napDNAbp may include any suitable homologs and/or orthologs or naturally occurring enzymes, such as Cas9. Cas9 homologs and/or orthologs have been described in various species, including, but not limited to, S. pyogenes and S. thermophilus. Preferably, the Cas moiety is configured (e.g, mutagenized, recombinantly engineered, or otherwise obtained from nature) as a nickase, i.e., capable of cleaving only a single strand of the target doubpdditional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase. In some embodiments, the Cas9 protein comprises an amino acid sequence that is at least 80% identical to the amino acid sequence of a Cas9 protein as provided by any one of the variants of Table 3. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the Cas9 orthologs in the above tables.

Dead napDNAbp Variants

In some embodiments, the disclosed base editors may comprise a catalytically inactive, or “dead,” napDNAbp domain. Exemplary catalytically inactive domains in the disclosed base editors are dead S. pyogenes Cas9 (dSpCas9) and S. pyogenes Cas9 nickase (SpCas9n).

In certain embodiments, the base editors described herein may include a dead Cas9, e.g., dead SpCas9, which has no nuclease activity due to one or more mutations that inactivate both nuclease domains of SpCas9, namely the RuvC domain (which cleaves the non-protospacer DNA strand) and HNH domain (which cleaves the protospacer DNA strand). The nuclease inactivation may be due to one or mutations that result in one or more substitutions and/or deletions in the amino acid sequence of the encoded protein, or any variants thereof having at least 80%, at least 85%, at least 90%, at least 95%, or at least 99% sequence identity thereto.

In certain embodiments, the base editors described herein may include a dead Cas9, e.g., dead SpCas9, which has no nuclease activity due to one or more mutations that inactivate both nuclease domains of SpCas9, namely the RuvC domain (which cleaves the non-protospacer DNA strand) and HNH domain (which cleaves the protospacer DNA strand). The D10A and N580A mutations in the wild-type S. aureus Cas9 amino acid sequence may be used to form a dSaCas9. Accordingly, in some embodiments, the napDNAbp domain of the base editors provided herein comprises a dSaCas9 that has D10A and N580A mutations relative to the wild-type SaCas9 sequence (SEQ ID NO: 216).

As used herein, the term “dCas9” refers to a nuclease-inactive Cas9 or nuclease-dead Cas9, or a functional fragment thereof, and embraces any naturally occurring dCas9 from any organism, any naturally-occurring dCas9 equivalent or functional fragment thereof, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and method for making dCas9 proteins are further described herein and/or are described in the art and are incorporated herein by reference.

In other embodiments, dCas9 corresponds to, or comprises in part or in whole, a Cas9 amino acid sequence having one or more mutations that inactivate the Cas9 nuclease activity. In other embodiments, Cas9 variants having mutations other than D10A and H840A are provided which may result in the full or partial inactivate of the endogenous Cas9 nuclease activity (e.g., nCas9 or dCas9, respectively). Such mutations, by way of example, include other amino acid substitutions at D10 and H820, or other substitutions within the nuclease domains of Cas9 (e.g., substitutions in the HNH nuclease subdomain and/or the RuvC1 subdomain) with reference to a wild type sequence such as Cas9 from Streptococcus pyogenes (NCBI Reference Sequence: NC_017053.1). In some embodiments, variants or homologues of Cas9 (e.g., variants of Cas9 from Streptococcus pyogenes (NCBI Reference Sequence: NC_017053.1)) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_017053.1. In some embodiments, variants of dCas9 (e.g., variants of NCBI Reference Sequence: NC_017053.1) are provided having amino acid sequences which are shorter, or longer than NC_017053.1 by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In some embodiments, the napDNAbp domain of any of the disclosed base editors comprises a dead S. pyogenes Cas9 (dSpCas9). In some embodiments, the napDNAbp domain of any of the disclosed based editors is comprises at least 80%, at least 85%, at least 90%, at least 95%, or at least 99% sequence identity to SEQ ID NO: 214. In some embodiments, the napDNAbp domain of any of the disclosed base editors comprises the amino acid sequence of SEQ ID NO: 214.

In one embodiment, the dead Cas9 may be based on the canonical SpCas9 sequence of Q99ZW2 and may have the following sequence, which comprises a D10A and an H810A substitutions (underlined and bolded), or a variant of SEQ ID NO: 214 having at least 80%, at least 85%, at least 90%, at least 95%, or at least 99% sequence identity thereto:

| | | SEQ ID |
|---|---|---|
| Description | Sequence | NO: |
| dead Cas9 or | MDKKYSIGL [symbol] IGTNSVGWAVITDEYKVPSKKFKVLGNTDRHSIKKNLIGALLFDSGETAEA | SEQ ID |
| dCas9 | TRLKRTARRRYTRRKNRICYLQEIFSNEMAKVDDSFFHRLEESFLVEEDKKHERHPIFGNI | NO: 20 |
| Streptococcus | VDEVAYHEKYPTIYHLRKKLVDSTDKADLRLIYLALAHMIKFRGHFLIEGDLNPDNSDVDK | |
| pyogenes | LFIQLVQTYNQLFEENPINASGVDAKAILSARLSKSRRLENLIAQLPGEKKNGLFGNLIAL | |
| Q99ZW2 Cas9 | SLGLTPNFKSNFDLAEDAKLQLSKDTYDDDLDNLLAQIGDQYADLFLAAKNLSDAILLSDI | |
| with D10 [symbol] and | LRVNTEITKAPLSASMIKRYDEHHQDLTLLKALVRQQLPEKYKEIFFDQSKNGYAGYIDGG | |
| H810 [symbol] | ASQEEFYKFIKPILEKMDGTEELLVKLNREDLLRKQRTFDNGSIPHQIHLGELHAILRRQE | |
| Where “X” is | DFYPFLKDNREKIEKILTFRIPYYVGPLARGNSRFAWMTRKSEETITPWNFEEVVDKGASA | |
| any amino | QSFIERMTNFDKNLPNEKVLPKHSLLYEYFTVYNELTKVKYVTEGMRKPAFLSGEQKKAIV | |
| acid | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVD [symbol] IVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| dead Cas9 or | MDKKYSIGL [symbol] IGTNSVGWAVITDEYKVPSKKFKVLGNTDRHSIKKNLIGALLFDSGETAEA | SEQ ID |
| dCas9 | TRLKRTARRRYTRRKNRICYLQEIFSNEMAKVDDSFFHRLEESFLVEEDKKHERHPIFGNI | NO: 214 |
| Streptococcus | VDEVAYHEKYPTIYHLRKKLVDSTDKADLRLIYLALAHMIKFRGHFLIEGDLNPDNSDVDK | |
| pyogenes | LFIQLVQTYNQLFEENPINASGVDAKAILSARLSKSRRLENLIAQLPGEKKNGLFGNLIAL | |
| Q99ZW2 Cas9 | SLGLTPNFKSNFDLAEDAKLQLSKDTYDDDLDNLLAQIGDQYADLFLAAKNLSDAILLSDI | |
| with D10 [symbol] and | LRVNTEITKAPLSASMIKRYDEHHQDLTLLKALVRQQLPEKYKEIFFDQSKNGYAGYIDGG | |
| H810 [symbol] | ASQEEFYKFIKPILEKMDGTEELLVKLNREDLLRKQRTFDNGSIPHQIHLGELHAILRRQE | |
| | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVD [symbol] IVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| dead | MSKLEKFTNCYSLSKTLRFKAIPVGKTQENIDNKRLLVEDEKRAEDYKGVKKLLDRYYLSF | SEQ ID |
| Lachnospiraceae | INDVLHSIKLKNLNNYISLFRKKTRTEKENKELENLEINLRKEIAKAFKGNEGYKSLFKKD | NO: 21 |
| bacterium | IIETILPEFLDDKDEIALVNSFNGFTTAFTGFFDNRENMFSEEAKSTSIAFRCINENLTRY | |
| Cas12a | ISNMDIFEKVDAIFDKHEVQEIKEKILNSDYDVEDFFEGEFFNFVLTQEGIDVYNAIIGGF | |
| | VTESGEKIKGLNEYINLYNQKTKQKLPKFKPLYKQVLSDRESLSFYGEGYTSDEEVLEVFR | |
| | NTLNKNSEIFSSIKKLEKLFKNFDEYSSAGIFVKNGPAISTISKDIFGEWNVIRDKWNAEY | |
| | DDIHLKKKAVVTEKYEDDRRKSFKKIGSFSLEQLQEYADADLSVVEKLKEIIIQKVDEIYK | |
| | VYGSSEKLFDADFVLEKSLKKNDAVVAIMKDLLDSVKSFENYIKAFFGEGKETNRDESFYG | |
| | DFVLAYDILLKVDHIYDAIRNYVTQKPYSKDKFKLYFQNPQFMGGWDKDKETDYRATILRY | |
| | GSKYYLAIMDKKYAKCLQKIDKDDVNGNYEKINYKLLPGPNKMLPKVFFSKKWMAYYNPSE | |
| | DIQKIYKNGTFKKGDMFNLNDCHKLIDFFKDSISRYPKWSNAYDFNFSETEKYKDIAGFYR | |
| | EVEEQGYKVSFESASKKEVDKLVEEGKLYMFQIYNKDFSDKSHGTPNLHTMYFKLLFDENN | |
| | HGQIRLSGGAELFMRRASLKKEELVVHPANSPIANKNPDNPKKTTTLSYDVYKDKRFSEDQ | |
| | YELHIPIAINKCPKNIFKINTEVRVLLKHDDNPYVIGIARGERNLLYIVVVDGKGNIVEQY | |
| | SLNEIINNFNGIRIKTDYHSLLDKKEKERFEARQNWTSIENIKELKAGYISQVVHKICELV | |
| | EKYDAVIALEDLNSGFKNSRVKVEKQVYQKFEKMLIDKLNYMVDKKSNPCATGGALKGYQI | |
| | TNKFESFKSMSTQNGFIFYIPAWLTSKIDPSTGFVNLLKTKYTSIADSKKFISSFDRIMYV | |
| | PEEDLFEFALDYKNFSRTDADYIKKWKLYSYGNRIRIFRNPKKNNVFDWEEVCLTSAYKEL | |
| | FNKYGINYQQGDIRALLCEQSDKAFYSSFMALMSLMLQMRNSITGRTDVDFLISPVKNSDG | |
| | IFYDSRNYEAQENAILPKNADANGAYNIARKVLWAIGQFKKAEDEKLDKVKIAISNKEWLE | |
| | YAQTSVK | |

napDNAbp Nickase Variants

In some embodiments, the disclosed base editors may comprise a napDNAbp domain that comprises a nickase. In some embodiments, the base editors described herein comprise a Cas9 nickase. The term “Cas9 nickase” of “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the RuvC domain could include D10X, H983X, D986X, or E762X, wherein X is any amino acid other than the wild type amino acid. In certain embodiments, the nickase could be D10A, of H983A, or D986A, or E762A, or a combination thereof.

In some embodiments, the napDNAbp domain of any of the disclosed base editors comprises an S. pyogenes Cas9 nickase (SpCas9n). In some embodiments, the napDNAbp domain of any of the disclosed based editors is comprises at least 80%, at least 85%, at least 90%, at least 95%, or at least 99% sequence identity to SEQ ID NO: 215 or 222. In some embodiments, the napDNAbp domain of any of the disclosed base editors comprises the amino acid sequence of SEQ ID NO: 215. In some embodiments, the napDNAbp domain of any of the disclosed base editors comprises the amino acid sequence of SEQ ID NO: 222.

In some embodiments, the napDNAbp domain of any of the disclosed base editors comprises an S. aureus Cas9 nickase (SaCas9n). In some embodiments, the napDNAbp domain of any of the disclosed based editors is comprises at least 80%, at least 85%, at least 90%, at least 95%, or at least 99% sequence identity to SEQ ID NO: 29. In some embodiments, the napDNAbp domain of any of the disclosed base editors comprises the amino acid sequence of SEQ ID NO:29.

In various embodiments, the Cas9 nickase can having a mutation in the RuvC nuclease domain and have one of the following amino acid sequences, or a variant thereof having an amino acid sequence that has at least 80%, at least 85%, at least 90%, at least 95%, or at least 99% sequence identity thereto.

| | | SEQ ID |
|---|---|---|
| Description | Sequence | NO: |
| Cas9 nickase | MDKKYSIGLXIGTNSVGWAVITDEYKVPSKKFKVLGNTDRHSIKKNLIGALLFDSGETAEA | SEQ ID |
| Streptococcus | TRLKRTARRRYTRRKNRICYLQEIFSNEMAKVDDSFFHRLEESFLVEEDKKHERHPIFGNI | NO: 22 |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with D10X, | SLGLTPNFKSNFDLAEDAKLQLSKDTYDDDLDNLLAQIGDQYADLFLAAKNLSDAILLSDI | |
| wherein X is | LRVNTEITKAPLSASMIKRYDEHHQDLTLLKALVRQQLPEKYKEIFFDQSKNGYAGYIDGG | |
| any alternate | ASQEEFYKFIKPILEKMDGTEELLVKLNREDLLRKQRTFDNGSIPHQIHLGELHAILRRQE | |
| amino acid | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | SEQ ID |
| Streptococcus | TRLKRTARRRYTRRKNRICYLQEIFSNEMAKVDDSFFHRLEESFLVEEDKKHERHPIFGNI | NO: 23 |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with E762X, | SLGLTPNFKSNFDLAEDAKLQLSKDTYDDDLDNLLAQIGDQYADLFLAAKNLSDAILLSDI | |
| wherein X is | LRVNTEITKAPLSASMIKRYDEHHQDLTLLKALVRQQLPEKYKEIFFDQSKNGYAGYIDGG | |
| any alternate | ASQEEFYKFIKPILEKMDGTEELLVKLNREDLLRKQRTFDNGSIPHQIHLGELHAILRRQE | |
| amino acid | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIXMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | SEQ ID |
| Streptococcus | TRLKRTARRRYTRRKNRICYLQEIFSNEMAKVDDSFFHRLEESFLVEEDKKHERHPIFGNI | NO: 24 |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with H983X, | SLGLTPNFKSNFDLAEDAKLQLSKDTYDDDLDNLLAQIGDQYADLFLAAKNLSDAILLSDI | |
| wherein X is | LRVNTEITKAPLSASMIKRYDEHHQDLTLLKALVRQQLPEKYKEIFFDQSKNGYAGYIDGG | |
| any alternate | ASQEEFYKFIKPILEKMDGTEELLVKLNREDLLRKQRTFDNGSIPHQIHLGELHAILRRQE | |
| amino acid | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHXAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | SEQ ID |
| Streptococcus | TRLKRTARRRYTRRKNRICYLQEIFSNEMAKVDDSFFHRLEESFLVEEDKKHERHPIFGNI | NO: 25 |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with D986X, | SLGLTPNFKSNFDLAEDAKLQLSKDTYDDDLDNLLAQIGDQYADLFLAAKNLSDAILLSDI | |
| wherein X is | LRVNTEITKAPLSASMIKRYDEHHQDLTLLKALVRQQLPEKYKEIFFDQSKNGYAGYIDGG | |
| any alternate | ASQEEFYKFIKPILEKMDGTEELLVKLNREDLLRKQRTFDNGSIPHQIHLGELHAILRRQE | |
| amino acid | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHXAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| Cas9 nickase | MDKKYSIGLAIGTNSVGWAVITDEYKVPSKKFKVLGNTDRHSIKKNLIGALLFDSGETAEA | SEQ ID |
| Streptococcus | TRLKRTARRRYTRRKNRICYLQEIFSNEMAKVDDSFFHRLEESFLVEEDKKHERHPIFGNI | NO: 215 |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with D10A | SLGLTPNFKSNFDLAEDAKLQLSKDTYDDDLDNLLAQIGDQYADLFLAAKNLSDAILLSDI | |
| | LRVNTEITKAPLSASMIKRYDEHHQDLTLLKALVRQQLPEKYKEIFFDQSKNGYAGYIDGG | |
| | ASQEEFYKFIKPILEKMDGTEELLVKLNREDLLRKQRTFDNGSIPHQIHLGELHAILRRQE | |
| | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | SEQ ID |
| Streptococcus | TRLKRTARRRYTRRKNRICYLQEIFSNEMAKVDDSFFHRLEESFLVEEDKKHERHPIFGNI | NO: 26 |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with E762A | SLGLTPNFKSNFDLAEDAKLQLSKDTYDDDLDNLLAQIGDQYADLFLAAKNLSDAILLSDI | |
| | LRVNTEITKAPLSASMIKRYDEHHQDLTLLKALVRQQLPEKYKEIFFDQSKNGYAGYIDGG | |
| | ASQEEFYKFIKPILEKMDGTEELLVKLNREDLLRKQRTFDNGSIPHQIHLGELHAILRRQE | |
| | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIAMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | SEQ ID |
| Streptococcus | TRLKRTARRRYTRRKNRICYLQEIFSNEMAKVDDSFFHRLEESFLVEEDKKHERHPIFGNI | NO: 27 |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with H983A | SLGLTPNFKSNFDLAEDAKLQLSKDTYDDDLDNLLAQIGDQYADLFLAAKNLSDAILLSDI | |
| | LRVNTEITKAPLSASMIKRYDEHHQDLTLLKALVRQQLPEKYKEIFFDQSKNGYAGYIDGG | |
| | ASQEEFYKFIKPILEKMDGTEELLVKLNREDLLRKQRTFDNGSIPHQIHLGELHAILRRQE | |
| | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHAAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | SEQ ID |
| Streptococcus | TRLKRTARRRYTRRKNRICYLQEIFSNEMAKVDDSFFHRLEESFLVEEDKKHERHPIFGNI | NO: 28 |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with D986A | SLGLTPNFKSNFDLAEDAKLQLSKDTYDDDLDNLLAQIGDQYADLFLAAKNLSDAILLSDI | |
| | LRVNTEITKAPLSASMIKRYDEHHQDLTLLKALVRQQLPEKYKEIFFDQSKNGYAGYIDGG | |
| | ASQEEFYKFIKPILEKMDGTEELLVKLNREDLLRKQRTFDNGSIPHQIHLGELHAILRRQE | |
| | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHAAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| Cas9 nickase | MGKRNYILGLAIGITSVGYGIIDYETRDVIDAGVRLFKEANVENNEGRRSKRGARRLKRRR | SEQ ID |
| Staphylococcus | RHRIQRVKKLLFDYNLLTDHSELSGINPYEARVKGLSQKLSEEEFSAALLHLAKRRGVHNV | NO: 29 |
| aureus | NEVEEDTGNELSTKEQISRNSKALEEKYVAELQLERLKKDGEVRGSINRFKTSDYVKEAKQ | |
| (SaCas9) | LLKVQKAYHQLDQSFIDTYIDLLETRRTYYEGPGEGSPFGWKDIKEWYEMLMGHCTYFPEE | |
| with D10A | LRSVKYAYNADLYNALNDLNNLVITRDENEKLEYYEKFQIIENVFKQKKKPTLKQIAKEIL | |
| | VNEEDIKGYRVTSTGKPEFTNLKVYHDIKDITARKEIIENAELLDQIAKILTIYQSSEDIQ | |
| | EELTNLNSELTQEEIEQISNLKGYTGTHNLSLKAINLILDELWHTNDNQIAIFNRLKLVPK | |
| | KVDLSQQKEIPTTLVDDFILSPVVKRSFIQSIKVINAIIKKYGLPNDIIIELAREKNSKDA | |
| | QKMINEMQKRNRQTNERIEEIIRTTGKENAKYLIEKIKLHDMQEGKCLYSLEAIPLEDLLN | |
| | NPFNYEVDHIIPRSVSFDNSFNNKVLVKQEENSKKGNRTPFQYLSSSDSKISYETFKKHIL | |
| | NLAKGKGRISKTKKEYLLEERDINRFSVQKDFINRNLVDTRYATRGLMNLLRSYFRVNNLD | |
| | VKVKSINGGFTSFLRRKWKFKKERNKGYKHHAEDALIIANADFIFKEWKKLDKAKKVMENQ | |
| | MFEEKQAESMPEIETEQEYKEIFITPHQIKHIKDFKDYKYSHRVDKKPNRKLINDTLYSTR | |
| | KDDKGNTLIVNNLNGLYDKDNDKLKKLINKSPEKLLMYHHDPQTYQKLKLIMEQYGDEKNP | |
| | LYKYYEETGNYLTKYSKKDNGPVIKKIKYYGNKLNAHLDITDDYPNSRNKVVKLSLKPYRF | |
| | DVYLDNGVYKFVTVKNLDVIKKENYYEVNSKCYEEAKKLKKISNQAEFIASFYKNDLIKIN | |
| | GELYRVIGVNNDLLNRIEVNMIDITYREYLENMNDKRPPHIIKTIASKTQSIKKYSTDILG | |
| | NLYEVKSKKHPQIIKK | |

In another embodiment, the Cas9 nickase comprises a mutation in the HNH domain which inactivates the HNH nuclease activity. For example, mutations in histidine (H) 840 or asparagine (R) 863 have been reported as loss-of-function mutations of the HNH nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the HNH domain could include H840X and R863X, wherein X is any amino acid other than the wild type amino acid. In certain embodiments, the nickase could be H840A or R863A or a combination thereof.

In various embodiments, the Cas9 nickase can have a mutation in the HNH nuclease domain and have one of the following amino acid sequences, or a variant thereof having an amino acid sequence that has at least 80%, at least 85%, at least 90%, at least 95%, or at least 99% sequence identity thereto.

| | | SEQ ID |
|---|---|---|
| Description | Sequence | NO: |
| Cas9 nickase | MDKKYSIGLDIGTNSVGWAVITDEYKVPSKKFKVLGNTDRHSIKKNLIGALLFDSGETAEA | SEQ ID |
| Streptococcus | TRLKRTARRRYTRRKNRICYLQEIFSNEMAKVDDSFFHRLEESFLVEEDKKHERHPIFGNI | NO: 30 |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with H840X, | SLGLTPNFKSNFDLAEDAKLQLSKDTYDDDLDNLLAQIGDQYADLFLAAKNLSDAILLSDI | |
| wherein X is | LRVNTEITKAPLSASMIKRYDEHHQDLTLLKALVRQQLPEKYKEIFFDQSKNGYAGYIDGG | |
| any alternate | ASQEEFYKFIKPILEKMDGTEELLVKLNREDLLRKQRTFDNGSIPHQIHLGELHAILRRQE | |
| amino acid | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDXIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | SEQ ID |
| Streptococcus | TRLKRTARRRYTRRKNRICYLQEIFSNEMAKVDDSFFHRLEESFLVEEDKKHERHPIFGNI | NO: 222 |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with H840A | SLGLTPNFKSNFDLAEDAKLQLSKDTYDDDLDNLLAQIGDQYADLFLAAKNLSDAILLSDI | |
| | LRVNTEITKAPLSASMIKRYDEHHQDLTLLKALVRQQLPEKYKEIFFDQSKNGYAGYIDGG | |
| | ASQEEFYKFIKPILEKMDGTEELLVKLNREDLLRKQRTFDNGSIPHQIHLGELHAILRRQE | |
| | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDAIVPQSFLKDDSIDN | |
| | KVLTRSDKNRGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | SEQ ID |
| Streptococcus | TRLKRTARRRYTRRKNRICYLQEIFSNEMAKVDDSFFHRLEESFLVEEDKKHERHPIFGNI | NO: 31 |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with R863X, | SLGLTPNFKSNFDLAEDAKLQLSKDTYDDDLDNLLAQIGDQYADLFLAAKNLSDAILLSDI | |
| wherein X is | LRVNTEITKAPLSASMIKRYDEHHQDLTLLKALVRQQLPEKYKEIFFDQSKNGYAGYIDGG | |
| any alternate | ASQEEFYKFIKPILEKMDGTEELLVKLNREDLLRKQRTFDNGSIPHQIHLGELHAILRRQE | |
| amino acid | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNXGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |
| Cas9 nickase | MDKKYSIGLDIGTNSVGWAVITDEYKVPSKKFKVLGNTDRHSIKKNLIGALLFDSGETAEA | SEQ ID |
| Streptococcus | TRLKRTARRRYTRRKNRICYLQEIFSNEMAKVDDSFFHRLEESFLVEEDKKHERHPIFGNI | NO: 32 |
| pyogenes | VDEVAYHEKYPTIYHLRKKLVDSTDKADLRLIYLALAHMIKFRGHFLIEGDLNPDNSDVDK | |
| Q99ZW2 Cas9 | LFIQLVQTYNQLFEENPINASGVDAKAILSARLSKSRRLENLIAQLPGEKKNGLFGNLIAL | |
| with R863A, | SLGLTPNFKSNFDLAEDAKLQLSKDTYDDDLDNLLAQIGDQYADLFLAAKNLSDAILLSDI | |
| wherein X is | LRVNTEITKAPLSASMIKRYDEHHQDLTLLKALVRQQLPEKYKEIFFDQSKNGYAGYIDGG | |
| any alternate | ASQEEFYKFIKPILEKMDGTEELLVKLNREDLLRKQRTFDNGSIPHQIHLGELHAILRRQE | |
| amino acid | DFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLDNE | |
| | ENEDILEDIVLTLTLFEDREMIEERLKTYAHLFDDKVMKQLKRRRYTGWGRLSRKLINGIR | |
| | DKQSGKTILDFLKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDN | |
| | KVLTRSDKNAGKSDNVPSEEVVKKMKNYWRQLLNAKLITQRKFDNLTKAERGGLSELDKAG | |
| | FIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVEKGKSKKLKS | |
| | VKELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTKEV | |
| | LDATLIHQSITGLYETRIDLSQLGGD | |

In some embodiments, the N-terminal methionine is removed from a Cas9 nickase, or from any Cas9 variant, ortholog, or equivalent disclosed or contemplated herein. For example, methionine-minus Cas9 nickases include the following sequences, or a variant thereof having an amino acid sequence that has at least 80%, at least 85%, at least 90%, at least 95%, or at least 99% sequence identity thereto.

| Description | Sequence |
|---|---|
| Cas9 nickase | DKKYSIGLDIGTNSVGWAVITDEYKVPSKKFKVLGNTDRHSIKKNLIGALLFDSGETAEATRLKRTARRRYTRRK |
| (Met minus) | NRICYLQEIFSNEMAKVDDSFFHRLEESFLVEEDKKHERHPIFGNIVDEVAYHEKYPTIYHLRKKLVDSTDKADL |
| Streptococcus | RLIYLALAHMIKFRGHFLIEGDLNPDNSDVDKLFIQLVQTYNQLFEENPINASGVDAKAILSARLSKSRRLENLI |
| pyogenes | AQLPGEKKNGLFGNLIALSLGLTPNFKSNFDLAEDAKLQLSKDTYDDDLDNLLAQIGDQYADLFLAAKNLSDAIL |
| Q99ZW2 Cas9 | LSDILRVNTEITKAPLSASMIKRYDEHHQDLTLLKALVRQQLPEKYKEIFFDQSKNGYAGYIDGGASQEEFYKFI |
| with H840X, | KPILEKMDGTEELLVKLNREDLLRKQRTFDNGSIPHQIHLGELHAILRRQEDFYPFLKDNREKIEKILTFRIPYY |
| wherein X is | VGPLARGNSRFAWMTRKSEETITPWNFEEVVDKGASAQSFIERMTNFDKNLPNEKVLPKHSLLYEYFTVYNELTK |
| any alternate | VKYVTEGMRKPAFLSGEQKKAIVDLLFKTNRKVTVKQLKEDYFKKIECFDSVEISGVEDRFNASLGTYHDLLKII |
| amino acid | KDKDFLDNEENEDILEDIVLTLTLFEDREMIEERLKTYAHLFDDKVMKQLKRRRYTGWGRLSRKLINGIRDKQSG |
| | KTILDFLKSDGFANRNFMQLIHDDSLTFKEDIQKAQVSGQGDSLHEHIANLAGSPAIKKGILQTVKVVDELVKVM |
| | GRHKPENIVIEMARENQTTQKGQKNSRERMKRIEEGIKELGSQILKEHPVENTQLQNEKLYLYYLQNGRDMYVDQ |
| | ELDINRLSDYDVDXIVPQSFLKDDSIDNKVLTRSDKNRGKSDNVPSEEVVKKMKNYWRQLLNAKLITQRKFDNLT |
| | KAERGGLSELDKAGFIKRQLVETRQITKHVAQILDSRMNTKYDENDKLIREVKVITLKSKLVSDFRKDFQFYKVR |
| | EINNYHHAHDAYLNAVVGTALIKKYPKLESEFVYGDYKVYDVRKMIAKSEQEIGKATAKYFFYSNIMNFFKTEIT |
| | LANGEIRKRPLIETNGETGEIVWDKGRDFATVRKVLSMPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVKKDLIIKLPKY |
| | SLFELENGRKRMLASAGELQKGNELALPSKYVNFLYLASHYEKLKGSPEDNEQKQLFVEQHKHYLDEIIEQISEF |
| | SKRVILADANLDKVLSAYNKHRDKPIREQAENIIHLFTLTNLGAPAAFKYFDTTIDRKRYTSTKEVLDATLIHQS |
| | ITGLYETRIDLSQLGGD (SEQ ID NO: 33) |
| Cas9 nickase | DKKYSIGLDIGTNSVGWAVITDEYKVPSKKFKVLGNTDRHSIKKNLIGALLFDSGETAEATRLKRTARRRYTRRK |
| (Met minus) | NRICYLQEIFSNEMAKVDDSFFHRLEESFLVEEDKKHERHPIFGNIVDEVAYHEKYPTIYHLRKKLVDSTDKADL |
| Streptococcus | RLIYLALAHMIKFRGHFLIEGDLNPDNSDVDKLFIQLVQTYNQLFEENPINASGVDAKAILSARLSKSRRLENLI |
| pyogenes | AQLPGEKKNGLFGNLIALSLGLTPNFKSNFDLAEDAKLQLSKDTYDDDLDNLLAQIGDQYADLFLAAKNLSDAIL |
| Q99ZW2 Cas9 | LSDILRVNTEITKAPLSASMIKRYDEHHQDLTLLKALVRQQLPEKYKEIFFDQSKNGYAGYIDGGASQEEFYKFI |
| with H840A, | KPILEKMDGTEELLVKLNREDLLRKQRTFDNGSIPHQIHLGELHAILRRQEDFYPFLKDNREKIEKILTFRIPYY |
| wherein X is | VGPLARGNSRFAWMTRKSEETITPWNFEEVVDKGASAQSFIERMTNFDKNLPNEKVLPKHSLLYEYFTVYNELTK |
| any alternate | VKYVTEGMRKPAFLSGEQKKAIVDLLFKTNRKVTVKQLKEDYFKKIECFDSVEISGVEDRFNASLGTYHDLLKII |
| amino acid | KDKDFLDNEENEDILEDIVLTLTLFEDREMIEERLKTYAHLFDDKVMKQLKRRRYTGWGRLSRKLINGIRDKQSG |
| | KTILDFLKSDGFANRNFMQLIHDDSLTFKEDIQKAQVSGQGDSLHEHIANLAGSPAIKKGILQTVKVVDELVKVM |
| | GRHKPENIVIEMARENQTTQKGQKNSRERMKRIEEGIKELGSQILKEHPVENTQLQNEKLYLYYLQNGRDMYVDQ |
| | ELDINRLSDYDVDAIVPQSFLKDDSIDNKVLTRSDKNRGKSDNVPSEEVVKKMKNYWRQLLNAKLITQRKFDNLT |
| | KAERGGLSELDKAGFIKRQLVETRQITKHVAQILDSRMNTKYDENDKLIREVKVITLKSKLVSDFRKDFQFYKVR |
| | EINNYHHAHDAYLNAVVGTALIKKYPKLESEFVYGDYKVYDVRKMIAKSEQEIGKATAKYFFYSNIMNFFKTEIT |
| | LANGEIRKRPLIETNGETGEIVWDKGRDFATVRKVLSMPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVKKDLIIKLPKY |
| | SLFELENGRKRMLASAGELQKGNELALPSKYVNFLYLASHYEKLKGSPEDNEQKQLFVEQHKHYLDEIIEQISEF |
| | SKRVILADANLDKVLSAYNKHRDKPIREQAENIIHLFTLTNLGAPAAFKYFDTTIDRKRYTSTKEVLDATLIHQS |
| | ITGLYETRIDLSQLGGD (SEQ ID NO: 34) |
| Cas9 nickase | DKKYSIGLDIGTNSVGWAVITDEYKVPSKKFKVLGNTDRHSIKKNLIGALLFDSGETAEATRLKRTARRRYTRRK |
| (Met minus) | NRICYLQEIFSNEMAKVDDSFFHRLEESFLVEEDKKHERHPIFGNIVDEVAYHEKYPTIYHLRKKLVDSTDKADL |
| Streptococcus | RLIYLALAHMIKFRGHFLIEGDLNPDNSDVDKLFIQLVQTYNQLFEENPINASGVDAKAILSARLSKSRRLENLI |
| pyogenes | AQLPGEKKNGLFGNLIALSLGLTPNFKSNFDLAEDAKLQLSKDTYDDDLDNLLAQIGDQYADLFLAAKNLSDAIL |
| Q99ZW2 Cas9 | LSDILRVNTEITKAPLSASMIKRYDEHHQDLTLLKALVRQQLPEKYKEIFFDQSKNGYAGYIDGGASQEEFYKFI |
| with R863X, | KPILEKMDGTEELLVKLNREDLLRKQRTFDNGSIPHQIHLGELHAILRRQEDFYPFLKDNREKIEKILTFRIPYY |
| wherein X is | VGPLARGNSRFAWMTRKSEETITPWNFEEVVDKGASAQSFIERMTNFDKNLPNEKVLPKHSLLYEYFTVYNELTK |
| any alternate | VKYVTEGMRKPAFLSGEQKKAIVDLLFKTNRKVTVKQLKEDYFKKIECFDSVEISGVEDRFNASLGTYHDLLKII |
| amino acid | KDKDFLDNEENEDILEDIVLTLTLFEDREMIEERLKTYAHLFDDKVMKQLKRRRYTGWGRLSRKLINGIRDKQSG |
| | KTILDFLKSDGFANRNFMQLIHDDSLTFKEDIQKAQVSGQGDSLHEHIANLAGSPAIKKGILQTVKVVDELVKVM |
| | GRHKPENIVIEMARENQTTQKGQKNSRERMKRIEEGIKELGSQILKEHPVENTQLQNEKLYLYYLQNGRDMYVDQ |
| | ELDINRLSDYDVDHIVPQSFLKDDSIDNKVLTRSDKNXGKSDNVPSEEVVKKMKNYWRQLLNAKLITQRKFDNLT |
| | KAERGGLSELDKAGFIKRQLVETRQITKHVAQILDSRMNTKYDENDKLIREVKVITLKSKLVSDFRKDFQFYKVR |
| | EINNYHHAHDAYLNAVVGTALIKKYPKLESEFVYGDYKVYDVRKMIAKSEQEIGKATAKYFFYSNIMNFFKTEIT |
| | LANGEIRKRPLIETNGETGEIVWDKGRDFATVRKVLSMPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVKKDLIIKLPKY |
| | SLFELENGRKRMLASAGELQKGNELALPSKYVNFLYLASHYEKLKGSPEDNEQKQLFVEQHKHYLDEIIEQISEF |
| | SKRVILADANLDKVLSAYNKHRDKPIREQAENIIHLFTLTNLGAPAAFKYFDTTIDRKRYTSTKEVLDATLIHQS |
| | ITGLYETRIDLSQLGGD(SEQ ID NO: 35) |
| Cas9 nickase | DKKYSIGLDIGTNSVGWAVITDEYKVPSKKFKVLGNTDRHSIKKNLIGALLFDSGETAEATRLKRTARRRYTRRK |
| (Met minus) | NRICYLQEIFSNEMAKVDDSFFHRLEESFLVEEDKKHERHPIFGNIVDEVAYHEKYPTIYHLRKKLVDSTDKADL |
| Streptococcus | RLIYLALAHMIKFRGHFLIEGDLNPDNSDVDKLFIQLVQTYNQLFEENPINASGVDAKAILSARLSKSRRLENLI |
| pyogenes | AQLPGEKKNGLFGNLIALSLGLTPNFKSNFDLAEDAKLQLSKDTYDDDLDNLLAQIGDQYADLFLAAKNLSDAIL |
| Q99ZW2 Cas9 | LSDILRVNTEITKAPLSASMIKRYDEHHQDLTLLKALVRQQLPEKYKEIFFDQSKNGYAGYIDGGASQEEFYKFI |
| with R863A, | KPILEKMDGTEELLVKLNREDLLRKQRTFDNGSIPHQIHLGELHAILRRQEDFYPFLKDNREKIEKILTFRIPYY |
| wherein X is | VGPLARGNSRFAWMTRKSEETITPWNFEEVVDKGASAQSFIERMTNFDKNLPNEKVLPKHSLLYEYFTVYNELTK |
| any alternate | VKYVTEGMRKPAFLSGEQKKAIVDLLFKTNRKVTVKQLKEDYFKKIECFDSVEISGVEDRFNASLGTYHDLLKII |
| amino acid | KDKDFLDNEENEDILEDIVLTLTLFEDREMIEERLKTYAHLFDDKVMKQLKRRRYTGWGRLSRKLINGIRDKQSG |
| | KTILDFLKSDGFANRNFMQLIHDDSLTFKEDIQKAQVSGQGDSLHEHIANLAGSPAIKKGILQTVKVVDELVKVM |
| | GRHKPENIVIEMARENQTTQKGQKNSRERMKRIEEGIKELGSQILKEHPVENTQLQNEKLYLYYLQNGRDMYVDQ |
| | ELDINRLSDYDVDHIVPQSFLKDDSIDNKVLTRSDKNAGKSDNVPSEEVVKKMKNYWRQLLNAKLITQRKFDNLT |
| | KAERGGLSELDKAGFIKRQLVETRQITKHVAQILDSRMNTKYDENDKLIREVKVITLKSKLVSDFRKDFQFYKVR |
| | EINNYHHAHDAYLNAVVGTALIKKYPKLESEFVYGDYKVYDVRKMIAKSEQEIGKATAKYFFYSNIMNFFKTEIT |
| | LANGEIRKRPLIETNGETGEIVWDKGRDFATVRKVLSMPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVKKDLIIKLPKY |
| | SLFELENGRKRMLASAGELQKGNELALPSKYVNFLYLASHYEKLKGSPEDNEQKQLFVEQHKHYLDEIIEQISEF |
| | SKRVILADANLDKVLSAYNKHRDKPIREQAENIIHLFTLTNLGAPAAFKYFDTTIDRKRYTSTKEVLDATLIHQS |
| | ITGLYETRIDLSQLGGD (SEQ ID NO: 36) |

### Other Cas9 Variants

The napDNAbp domains used in the base editors described herein may also include other Cas9 variants that area at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to a reference Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 99.5% of the amino acid length of a corresponding wild type Cas9 (e.g., SEQ ID NO: 213).

In some embodiments, the disclosure also may utilize Cas9 fragments which retain their functionality and which are fragments of any herein disclosed Cas9 protein. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or 1300 amino acids in length.

In various embodiments, the base editors disclosed herein may comprise one of the Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

### Other Cas9 Equivalents

In some embodiments, the base editors described herein can include any Cas9 equivalent. As used herein, the term “Cas9 equivalent” is a broad term that encompasses any napDNAbp protein that serves the same function as Cas9 in the present base editors despite that its amino acid primary sequence and/or its three-dimensional structure may be different and/or unrelated from an evolutionary standpoint. Thus, while Cas9 equivalents include any Cas9 ortholog, homolog, mutant, or variant described or embraced herein that are evolutionarily related, the Cas9 equivalents also embrace proteins that may have evolved through convergent evolution processes to have the same or similar function as Cas9, but which do not necessarily have any similarity with regard to amino acid sequence and/or three dimensional structure. The base editors described here embrace any Cas9 equivalent that would provide the same or similar function as Cas9 despite that the Cas9 equivalent may be based on a protein that arose through convergent evolution.

For example, CasX is a Cas9 equivalent that reportedly has the same function as Cas9 but which evolved through convergent evolution. Thus, the CasX protein described in Liu et al., “CasX enzymes comprises a distinct family of RNA-guided genome editors,” Nature, 2019, Vol. 566: 218-223, is contemplated to be used with the base editors described herein. In addition, any variant or modification of CasX is conceivable and within the scope of the present disclosure.

Cas9 is a bacterial enzyme that evolved in a wide variety of species. However, the Cas9 equivalents contemplated herein may also be obtained from archaea, which constitute a domain and kingdom of single-celled prokaryotic microbes different from bacteria.

In some embodiments, Cas9 equivalents may refer to CasX or CasY, which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a nucleic acid programmable DNA binding protein (napDNAbp), and are within the scope of this disclosure. Also see Liu et al., “CasX enzymes comprises a distinct family of RNA-guided genome editors,” Nature, 2019, Vol. 566: 218-223. Any of these Cas9 equivalents are contemplated.

In some embodiments, the Cas9 equivalent comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring CasX or CasY protein. In some embodiments, the napDNAbp is a naturally-occurring CasX or CasY protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein.

In various embodiments, the nucleic acid programmable DNA binding proteins include, without limitation, Cas9 (e.g., dCas9 and nCas9), CasX, CasY, Cpf1, C2c1, C2c2, C2C3, Argonaute, Cas12a, and Cas12b. One example of a nucleic acid programmable DNA-binding protein that has different PAM specificity than Cas9 is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference. The state of the art may also now refer to Cpf1 enzymes as Cas12a.

In still other embodiments, the Cas protein may include any CRISPR associated protein, including but not limited to Cas12a, Cas12b, Cas1, Cas1B, Cas2, Cas3, Cas4, Cas5, Cas6, Cas7, Cas8, Cas9 (sometimes referred to as Csn1 and Csx12), Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type SpCas9 polypeptide of SEQ ID NO: 213).

In various other embodiments, the napDNAbp can be any of the following proteins: a Cas9, a Cpf1, a CasX, a CasY, a C2c1, a C2c2, a C2c3, a GeoCas9, a CjCas9, a Cas12a, a Cas12b, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago), a Cas9-KKH, a SmacCas9, a Spy-macCas9, an SpCas9-VRQR, an SpCas9-NRRH, an SpaCas9-NRTH, an SpCas9-NRCH, an LbCas12a, an AsCas12a, a CeCas12a, an MbCas12a, Cas3, CasΦ, and circularly permuted Cas9, domains such as CP1012, CP1028, CP1041, CP1249, and CP1300.

In certain embodiments, the base editors contemplated herein can include a Cas9 protein that is of smaller molecular weight than the canonical SpCas9 sequence. In some embodiments, the smaller-sized Cas9 variants may facilitate delivery to cells, e.g., by an expression vector, nanoparticle, or other means of delivery. The canonical SpCas9 protein is 1368 amino acids in length and has a predicted molecular weight of 158 kilodaltons. The term “small-sized Cas9 variant”, as used herein, refers to any Cas9 variant-naturally occurring, engineered, or otherwise that is less than at least 1300 amino acids, or at least less than 1290 amino acids, or than less than 1280 amino acids, or less than 1270 amino acid, or less than 1260 amino acid, or less than 1250 amino acids, or less than 1240 amino acids, or less than 1230 amino acids, or less than 1220 amino acids, or less than 1210 amino acids, or less than 1200 amino acids, or less than 1190 amino acids, or less than 1180 amino acids, or less than 1170 amino acids, or less than 1160 amino acids, or less than 1150 amino acids, or less than 1140 amino acids, or less than 1130 amino acids, or less than 1120 amino acids, or less than 1110 amino acids, or less than 1100 amino acids, or less than 1050 amino acids, or less than 1000 amino acids, or less than 950 amino acids, or less than 900 amino acids, or less than 850 amino acids, or less than 800 amino acids, or less than 750 amino acids, or less than 700 amino acids, or less than 650 amino acids, or less than 600 amino acids, or less than 550 amino acids, or less than 500 amino acids, but at least larger than about 400 amino acids and retaining the required functions of the Cas9 protein.

In some embodiments, Cas9 equivalents may refer to a Cas3, which has been described in, Morisaka et al., “CRISPR-Cas3 induces broad and unidirectional genome editing in human cells,” Nature Comm. (2019) 10:5302, which is hereby incorporated by reference. Cas3, which exhibits helicase as well as endonuclease activity, was shown to successfully cleave target sequences in genomic DNA in human cells in vitro.

In some embodiments, Cas9 equivalents may refer to a CasΦ, which has been described in Pausch et al., Science (2020) 369:6501, 333-337, which is hereby incorporated by reference. CasΦ uses a single active site for both CRISPR RNA (crRNA) processing and crRNA-guided DNA cutting to target foreign nucleic acids, and may have expanded target recognition capabilities relative to other CRISPR-Cas proteins.

In various embodiments, the base editors disclosed herein may comprise one of the small-sized Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference small-sized Cas9 protein. Exemplary small-sized Cas9 variants include, but are not limited to, SaCas9, LbCas12a, AsCas12a, CeCas12a, and MbCas12a. For instance, Chen et al. recently showed that a novel Cas12a nuclease CeCas12a from Coprococcus eutactus, a napDNAbp with editing efficiencies comparable to AsCas12a and LbCas12a in human cells, Moreover, had higher stringenices for PAM recognition in vitro and in vivo followed by very low off-target editing rates in cells. Importantly, CeCas12a rendered less off-target edits located at C-containing PAM at multiple sites compared to LbCas12a and AsCas12a, as assessed by targeted sequencing methods. See Chen et al., Genome Biol. 2020; 21:78, herein incorporated by reference.

In some embodiments, the base editors described herein may also comprise Cas12a/Cpf1 (dCpf1) variants that may be used as a guide nucleotide sequence-programmable DNA-binding protein domain. The Cas12a/Cpf1 protein has a RuvC-like endonuclease domain that is similar to the RuvC domain of Cas9 but does not have an HNH endonuclease domain, and the N-terminal of Cpf1 does not have the alpha-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity.

In some embodiments, the nucleic acid programmable DNA binding protein (napDNAbp) of any of the CBEs provided herein may be a C2c1, a C2c2, or a C2c3 protein. In some embodiments, the napDNAbp is a C2c1 protein. In some embodiments, the napDNAbp is a C2c2 protein. In some embodiments, the napDNAbp is a C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring C2c1, C2c2, or C2c3 protein. In some embodiments, the napDNAbp is a naturally-occurring C2c1, C2c2, or C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to one of SEQ ID NOs: 220 or 221. In some embodiments, the napDNAbp comprises an amino acid sequence of SEQ ID NOs: 220 or 221. It should be appreciated that C2c1, C2c2, or C2c3 from other bacterial species may also be used in accordance with the present disclosure.

In some embodiments, the nucleic acid programmable DNA binding protein (napDNAbp) of any of the CBEs provided herein may be a CjCas9, Cas12a, Cas12b, Cas12g, Cas12h, Cas12i, Cas13b, Cas13c, Cas13d, Cas14, Csn2, and GeoCas9. CjCas9 is described and characterized in Kim et al., Nat Commun. 2017; 8:14500 and Dugar et al., Molecular Cell 2018; 69:893-905, incorporated herein by reference. GeoCas9 is described and characterized in Harrington et al. Nat Commun. 2017; 8(1):1424 and International Publication No. PCT/US2019/58678, filed Oct. 29, 2019, which published as International Publication No. WO 2020/092453 on May 7, 2020, each of incorporated herein by reference. The Cas12a, Cas12b, Cas12g, Cas12h and Cas12i proteins are described and characterized in, e.g., Yan et al., Science, 2019; 363(6422): 88-91, Murugan et al. The Revolution Continues: Newly Discovered Systems Expand the CRISPR-Cas Toolkit, Molecular Cell 2017; 68(1):15-25, each of which are incorporated herein by reference. Cas14 is characterized and described in Harrington et al. Science 2018; 362(6416):839-842, incorporated herein by reference. Cas13b, Cas13c and Cas13d are described and characterized in Smargon et al., Molecular Cell 2017, Cox et al., Science 2017, and Yan et al. Molecular Cell 70, 327-339.e5 (2018), each of which are incorporated herein by reference. Csn2 is described and characterized in Koo Y., Jung D. K., and Bae E. PloS One. 2012; 7:e33401, incorporated herein by reference.

```
C2cl (uniprot.org/uniprot/T0D7A2#)
sp|T0D7A2|C2Cl_ALIAG CRISPR-associated endonuclease
C2c1 OS = Alicyclobacillus acidoterrestris
(strain ATCC 49025/DSM 3922/CIP 106132/NCIMB 13137/GD3B)
GN = c2c1 PE = 1 SV = 1
MAVKSIKVKLRLDDMPEIRAGLWKLHKEVNAGVRYYTEWLSLLRQENLYR
RSPNGDGEQECDKTAEECKAELLERLRARQVENGHRGPAGSDDELLQLAR
QLYELLVPQAIGAKGDAQQIARKFLSPLADKDAVGGLGIAKAGNKPRWVR
MREAGEPGWEEEKEKAETRKSADRTADVLRALADFGLKPLMRVYTDSEMS
SVEWKPLRKGQAVRTWDRDMFQQAIERMMSWESWNQRVGQEYAKLVEQKN
RFEQKNFVGQEHLVHLVNQLQQDMKEASPGLESKEQTAHYVTGRALRGSD
KVFEKWGKLAPDAPFDLYDAEIKNVQRRNTRRFGSHDLFAKLAEPEYQAL
WREDASFLTRYAVYNSILRKLNHAKMFATFTLPDATAHPIWTRFDKLGGN
LHQYTFLFNEFGERRHAIRFHKLLKVENGVAREVDDVTVPISMSEQLDNL
LPRDPNEPIALYFRDYGAEQHFTGEFGGAKIQCRRDQLAHMHRRRGARDV
YLNVSVRVQSQSEARGERRPPYAAVFRLVGDNHRAFVHFDKLSDYLAEHP
DDGKLGSEGLLSGLRVMSVDLGLRTSASISVFRVARKDELKPNSKGRVPF
FFPIKGNDNLVAVHERSQLLKLPGETESKDLRAIREERQRTLRQLRTQLA
YLRLLVRCGSEDVGRRERSWAKLIEQPVDAANHMTPDWREAFENELQKLK
SLHGICSDKEWMDAVYESVRRVWRHMGKQVRDWRKDVRSGERPKIRGYAK
DVVGGNSIEQIEYLERQYKFLKSWSFFGKVSGQVIRAEKGSRFAITLREH
IDHAKEDRLKKLADRIIMEALGYVYALDERGKGKWVAKYPPCQLILLEEL
SEYQFNNDRPPSENNQLMQWSHRGVFQELINQAQVHDLLVGTMYAAFSSR
FDARTGAPGIRCRRVPARCTQEHNPEPFPWWLNKFVVEHTLDACPLRADD
LIPTGEGEIFVSPFSAEEGDFHQIHADLNAAQNLQQRLWSDFDISQIRLR
CDWGEVDGELVLIPRLTGKRTADSYSNKVFYTNTGVTYYERERGKKRRKV
FAQEKLSEEEAELLVEADEAREKSVVLMRDPSGIINRGNWTRQKEFWSMV
NQRIEGYLVKQIRSRVPLQDSACENTGDI
(SEQ ID NO: 220)
  C2c2 (uniprot.org/uniprot/PODOC6)
>SP|P0DOC6|C2C2_LEPSD CRISPR-ASSOCIATED
ENDORIBONUCLEASE C2C2 OS = LEPTOTRICHIA SHAHII
(STRAIN DSM 19757/CCUG 47503/CIP 107916/
JCM 16776/LB37) GN = C2C2 PE = 1 SV = 1
MGNLFGHKRWYEVRDKKDFKIKRKVKVKRNYDGNKYILNINENNNKEKID
NNKFIRKYINYKKNDNILKEFTRKFHAGNILFKLKGKEGIIRIENNDDFL
ETEEVVLYIEAYGKSEKLKALGITKKKIIDEAIRQGITKDDKKIEIKRQE
NEEEIEIDIRDEYTNKTLNDCSIILRIIENDELETKKSIYEIFKNINMSL
YKIIEKIIENETEKVFENRYYEEHLREKLLKDDKIDVILTNFMEIREKIK
SNLEILGFVKFYLNVGGDKKKSKNKKMLVEKILNINVDLTVEDIADFVIK
ELEFWNITKRIEKVKKVNNEFLEKRRNRTYIKSYVLLDKHEKFKIERENK
KDKIVKFFVENIKNNSIKEKIEKILAEFKIDELIKKLEKELKKGNCDTEI
FGIFKKHYKVNFDSKKFSKKSDEEKELYKIIYRYLKGRIEKILVNEQKVR
LKKMEKIEIEKILNESILSEKILKRVKQYTLEHIMYLGKLRHNDIDMTTV
NTDDFSRLHAKEELDLELITFFASTNMELNKIFSRENINNDENIDFFGGD
REKNYVLDKKILNSKIKIIRDLDFIDNKNNITNNFIRKFTKIGTNERNRI
LHAISKERDLQGTQDDYNKVINIIQNLKISDEEVSKALNLDVVFKDKKNI
ITKINDIKISEENNNDIKYLPSFSKVLPEILNLYRNNPKNEPFDTIETEK
IVLNALIYVNKELYKKLILEDDLEENESKNIFLQELKKTLGNIDEIDENI
IENYYKNAQISASKGNNKAIKKYQKKVIECYIGYLRKNYEELFDFSDFKM
NIQEIKKQIKDINDNKTYERITVKTSDKTIVINDDFEYIISIFALLNSNA
VINKIRNRFFATSVWLNTSEYQNIIDILDEIMQLNTLRNECITENWNLNL
EEFIQKMKEIEKDFDDFKIQTKKEIFNNYYEDIKNNILTEFKDDINGCDV
LEKKLEKIVIFDDETKFEIDKKSNILQDEQRKLSNINKKDLKKKVDQYIK
DKDQEIKSKILCRIIFNSDFLKKYKKEIDNLIEDMESENENKFQEIYYPK
ERKNELYIYKKNLFLNIGNPNFDKIYGLISNDIKMADAKFLFNIDGKNIR
KNKISEIDAILKNLNDKLNGYSKEYKEKYIKKLKENDDFFAKNIQNKNYK
SFEKDYNRVSEYKKIRDLVEFNYLNKIESYLIDINWKLAIQMARFERDMH
YIVNGLRELGIIKLSGYNTGISRAYPKRNGSDGFYTTTAYYKFFDEESYK
KFEKICYGFGIDLSENSEINKPENESIRNYISHFYIVRNPFADYSIAEQI
DRVSNLLSYSTRYNNSTYASVFEVFKKDVNLDYDELKKKFKLIGNNDILE
RLMKPKKVSVLELESYNSDYIKNLIIELLTKIENTNDTL
  (SEQ ID NO: 221)
```

| Description | Sequence | SEQ ID NO: |
|---|---|---|
| SaCas9 | MGKRNYILGLDIGITSVGYGIIDYETRDVIDAGVRLFKEANVENNEGRRSKRGARRLKRRR | SEQ ID NO: 37 |
| Staphylococcus | RHRIQRVKKLLFDYNLLTDHSELSGINPYEARVKGLSQKLSEEEFSAALLHLAKRRGVHNV | |
| aureus | NEVEEDTGNELSTKEQISRNSKALEEKYVAELQLERLKKDGEVRGSINRFKTSDYVKEAKQ | |
| 1053 AA | LLKVQKAYHQLDQSFIDTYIDLLETRRTYYEGPGEGSPFGWKDIKEWYEMLMGHCTYFPEE | |
| 123 kDa | LRSVKYAYNADLYNALNDLNNLVITRDENEKLEYYEKFQIIENVFKQKKKPTLKQIAKEIL | |
| | VNEEDIKGYRVTSTGKPEFTNLKVYHDIKDITARKEIIENAELLDQIAKILTIYQSSEDIQ | |
| | EELTNLNSELTQEEIEQISNLKGYTGTHNLSLKAINLILDELWHTNDNQIAIFNRLKLVPK | |
| | KVDLSQQKEIPTTLVDDFILSPVVKRSFIQSIKVINAIIKKYGLPNDIIIELAREKNSKDA | |
| | QKMINEMQKRNRQTNERIEEIIRTTGKENAKYLIEKIKLHDMQEGKCLYSLEAIPLEDLLN | |
| | NPFNYEVDHIIPRSVSFDNSFNNKVLVKQEENSKKGNRTPFQYLSSSDSKISYETFKKHIL | |
| | NLAKGKGRISKTKKEYLLEERDINRFSVQKDFINRNLVDTRYATRGLMNLLRSYFRVNNLD | |
| | VKVKSINGGFTSFLRRKWKFKKERNKGYKHHAEDALIIANADFIFKEWKKLDKAKKVMENQ | |
| | MFEEKQAESMPEIETEQEYKEIFITPHQIKHIKDFKDYKYSHRVDKKPNRKLINDTLYSTR | |
| | KDDKGNTLIVNNLNGLYDKDNDKLKKLINKSPEKLLMYHHDPQTYQKLKLIMEQYGDEKNP | |
| | LYKYYEETGNYLTKYSKKDNGPVIKKIKYYGNKLNAHLDITDDYPNSRNKVVKLSLKPYRF | |
| | DVYLDNGVYKFVTVKNLDVIKKENYYEVNSKCYEEAKKLKKISNQAEFIASFYKNDLIKIN | |
| | GELYRVIGVNNDLLNRIEVNMIDITYREYLENMNDKRPPHIIKTIASKTQSIKKYSTDILG | |
| | NLYEVKSKKHPQIIKK | |
| NmeCas 9 | MAAFKPNSINYILGLDIGIASVGWAMVEIDEEENPIRLIDLGVRVFERAEVPKTGDSLAMA | SEQ ID NO: 38 |
| N. | RRLARSVRRLTRRRAHRLLRTRRLLKREGVLQAANFDENGLIKSLPNTPWQLRAAALDRKL | |
| meningitidis | TPLEWSAVLLHLIKHRGYLSQRKNEGETADKELGALLKGVAGNAHALQTGDFRTPAELALN | |
| 1083 AA | KFEKESGHIRNQRSDYSHTFSRKDLQAELILLFEKQKEFGNPHVSGGLKEGIETLLMTQRP | |
| 124.5 kDa | ALSGDAVQKMLGHCTFEPAEPKAAKNTYTAERFIWLTKLNNLRILEQGSERPLTDTERATL | |
| | MDEPYRKSKLTYAQARKLLGLEDTAFFKGLRYGKDNAEASTLMEMKAYHAISRALEKEGLK | |
| | DKKSPLNLSPELQDEIGTAFSLFKTDEDITGRLKDRIQPEILEALLKHISFDKFVQISLKA | |
| | LRRIVPLMEQGKRYDEACAEIYGDHYGKKNTEEKIYLPPIPADEIRNPVVLRALSQARKVI | |
| | NGVVRRYGSPARIHIETAREVGKSFKDRKEIEKRQEENRKDREKAAAKFREYFPNFVGEPK | |
| | SKDILKLRLYEQQHGKCLYSGKEINLGRLNEKGYVEIDAALPFSRTWDDSFNNKVLVLGSE | |
| | NQNKGNQTPYEYFNGKDNSREWQEFKARVETSRFPRSKKQRILLQKFDEDGFKERNLNDTR | |
| | YVNRFLCQFVADRMRLTGKGKKRVFASNGQITNLLRGFWGLRKVRAENDRHHALDAVVVAC | |
| | STVAMQQKITRFVRYKEMNAFDGKTIDKETGEVLHQKTHFPQPWEFFAQEVMIRVFGKPDG | |
| | KPEFEEADTLEKLRTLLAEKLSSRPEAVHEYVTPLFVSRAPNRKMSGQGHMETVKSAKRLD | |
| | EGVSVLRVPLTQLKLKDLEKMVNREREPKLYEALKARLEAHKDDPAKAFAEPFYKYDKAGN | |
| | RTQQVKAVRVEQVQKTGVWVRNHNGIADNATMVRVDVFEKGDKYYLVPIYSWQVAKGILPD | |
| | RAVVQGKDEEDWQLIDDSFNFKFSLHPNDLVEVITKKARMFGYFASCHRGTGNINIRIHDL | |
| | DHKIGKNGILEGIGVKTALSFQKYQIDELGKEIRPCRLKKRPPVR | |
| CjCas9 | MARILAFDIGISSIGWAFSENDELKDCGVRIFTKVENPKTGESLALPRRLARSARKRLARR | SEQ ID NO: 39 |
| C. jejuni | KARLNHLKHLIANEFKLNYEDYQSFDESLAKAYKGSLISPYELRFRALNELLSKQDFARVI | |
| 984 AA | LHIAKRRGYDDIKNSDDKEKGAILKAIKQNEEKLANYQSVGEYLYKEYFQKFKENSKEFTN | |
| 114.9 kDa | VRNKKESYERCIAQSFLKDELKLIFKKQREFGFSFSKKFEEEVLSVAFYKRALKDFSHLVG | |
| | NCSFFTDEKRAPKNSPLAFMFVALTRIINLLNNLKNTEGILYTKDDLNALLNEVLKNGTLT | |
| | YKQTKKLLGLSDDYEFKGEKGTYFIEFKKYKEFIKALGEHNLSQDDLNEIAKDITLIKDEI | |
| | KLKKALAKYDLNQNQIDSLSKLEFKDHLNISFKALKLVTPLMLEGKKYDEACNELNLKVAI | |
| | NEDKKDFLPAFNETYYKDEVTNPVVLRAIKEYRKVLNALLKKYGKVHKINIELAREVGKNH | |
| | SQRAKIEKEQNENYKAKKDAELECEKLGLKINSKNILKLRLFKEQKEFCAYSGEKIKISDL | |
| | QDEKMLEIDHIYPYSRSFDDSYMNKVLVFTKQNQEKLNQTPFEAFGNDSAKWQKIEVLAKN | |
| | LPTKKQKRILDKNYKDKEQKNFKDRNLNDTRYIARLVLNYTKDYLDFLPLSDDENTKLNDT | |
| | QKGSKVHVEAKSGMLTSALRHTWGFSAKDRNNHLHHAIDAVIIAYANNSIVKAFSDFKKEQ | |
| | ESNSAELYAKKISELDYKNKRKFFEPFSGFRQKVLDKIDEIFVSKPERKKPSGALHEETFR | |
| | KEEEFYQSYGGKEGVLKALELGKIRKVNGKIVKNGDMFRVDIFKHKKTNKFYAVPIYTMDF | |
| | ALKVLPNKAVARSKKGEIKDWILMDENYEFCFSLYKDSLILIQTKDMQEPEFVYYNAFTSS | |
| | TVSLIVSKHDNKFETLSKNQKILFKNANEKEVIAKSIGIQNLKVFEKYIVSALGEVTKAEF | |
| | RQREDFKK | |
| GeoCasS | MRYKIGLDIGITSVGWAVMNLDIPRIEDLGVRIFDRAENPQTGESLALPRRLARSARRRLR | SEQ ID NO: 40 |
| G. | RRKHRLERIRRLVIREGILTKEELDKLFEEKHEIDVWQLRVEALDRKLNNDELARVLLHLA | |
| stearothermo | KRRGFKSNRKSERSNKENSTMLKHIEENRAILSSYRTVGEMIVKDPKFALHKRNKGENYTN | |
| philus | TIARDDLEREIRLIFSKQREFGNMSCTEEFENEYITIWASQRPVASKDDIEKKVGFCTFEP | |
| | KEKRAPKATYTFQSFIAWEHINKLRLISPSGARGLTDEERRLLYEQAFQKNKITYHDIRTL | |
| 1087 AA | LHLPDDTYFKGIVYDRGESRKQNENIRFLELDAYHQIRKAVDKVYGKGKSSSFLPIDFDTF | |
| 127 kDa | GYALTLFKDDADIHSYLRNEYEQNGKRMPNLANKVYDNELIEELLNLSFTKFGHLSLKALR | |
| | SILPYMEQGEVYSSACERAGYTFTGPKKKQKTMLLPNIPPIANPVVMRALTQARKVVNAI1 | |
| | KKYGSPVSIHIELARDLSQTFDERRKTKKEQDENRKKNETAIRQLMEYGLTLNPTGHDIVK | |
| | FKLWSEQNGRCAYSLQPIEIERLLEPGYVEVDHVIPYSRSLDDSYTNKVLVLTRENREKGN | |
| | RIPAEYLGVGTERWQQFETFVLTNKQFSKKKRDRLLRLHYDENEETEFKNRNLNDTRYISR | |
| | FFANFIREHLKFAESDDKQKVYTVNGRVTAHLRSRWEFNKNREESDLHHAVDAVIVACTTP | |
| | SDIAKVTAFYQRREQNKELAKKTEPHFPQPWPHFADELRARLSKHPKESIKALNLGNYDDQ | |
| | KLESLQPVFVSRMPKRSVTGAAHQETLRRYVGIDERSGKIQTVVKTKLSEIKLDASGHFPM | |
| | YGKESDPRTYEAIRQRLLEHNNDPKKAFQEPLYKPKKNGEPGPVIRTVKIIDTKNQVIPLN | |
| | DGKTVAYNSNIVRVDVFEKDGKYYCVPVYTMDIMKGILPNKAIEPNKPYSEWKEMTEDYTF | |
| | RFSLYPNDLIRIELPREKTVKTAAGEEINVKDVFVYYKTIDSANGGLELISHDHRFSLRGV | |
| | GSRTLKRFEKYQVDVLGNIYKVRGEKRVGLASSAHSKPGKTIRPLQSTRD | |
| LbCas12a | MSKLEKFTNCYSLSKTLRFKAIPVGKTQENIDNKRLLVEDEKRAEDYKGVKKLLDRYYLSF | SEQ ID NO: 41 |
| L. bacterium | INDVLHSIKLKNLNNYISLFRKKTRTEKENKELENLEINLRKEIAKAFKGNEGYKSLFKKD | |
| 1228 AA | IIETILPEFLDDKDEIALVNSFNGFTTAFTGFFDNRENMFSEEAKSTSIAFRCINENLTRY | |
| 143.9 kDa | ISNMDIFEKVDAIFDKHEVQEIKEKILNSDYDVEDFFEGEFFNFVLTQEGIDVYNAIIGGF | |
| | VTESGEKIKGLNEYINLYNQKTKQKLPKFKPLYKQVLSDRESLSFYGEGYTSDEEVLEVFR | |
| | NTLNKNSEIFSSIKKLEKLFKNFDEYSSAGIFVKNGPAISTISKDIFGEWNVIRDKWNAEY | |
| | DDIHLKKKAVVTEKYEDDRRKSFKKIGSFSLEQLQEYADADLSVVEKLKEIIIQKVDEIYK | |
| | VYGSSEKLFDADFVLEKSLKKNDAVVAIMKDLLDSVKSFENYIKAFFGEGKETNRDESFYG | |
| | DFVLAYDILLKVDHIYDAIRNYVTQKPYSKDKFKLYFQNPQFMGGWDKDKETDYRATILRY | |
| | GSKYYLAIMDKKYAKCLQKIDKDDVNGNYEKINYKLLPGPNKMLPKVFFSKKWMAYYNPSE | |
| | DIQKIYKNGTFKKGDMFNLNDCHKLIDFFKDSISRYPKWSNAYDFNFSETEKYKDIAGFYR | |
| | EVEEQGYKVSFESASKKEVDKLVEEGKLYMFQIYNKDFSDKSHGTPNLHTMYFKLLFDENN | |
| | HGQIRLSGGAELFMRRASLKKEELVVHPANSPIANKNPDNPKKTTTLSYDVYKDKRFSEDQ | |
| | YELHIPIAINKCPKNIFKINTEVRVLLKHDDNPYVIGIDRGERNLLYIVVVDGKGNIVEQY | |
| | SLNEIINNFNGIRIKTDYHSLLDKKEKERFEARQNWTSIENIKELKAGYISQVVHKICELV | |
| | EKYDAVIALEDLNSGFKNSRVKVEKQVYQKFEKMLIDKLNYMVDKKSNPCATGGALKGYQI | |
| | TNKFESFKSMSTQNGFIFYIPAWLTSKIDPSTGFVNLLKTKYTSIADSKKFISSFDRIMYV | |
| | PEEDLFEFALDYKNFSRTDADYIKKWKLYSYGNRIRIFRNPKKNNVFDWEEVCLTSAYKEL | |
| | FNKYGINYQQGDIRALLCEQSDKAFYSSFMALMSLMLQMRNSITGRTDVDFLISPVKNSDG | |
| | IFYDSRNYEAQENAILPKNADANGAYNIARKVLWAIGQFKKAEDEKLDKVKIAISNKEWLE | |
| | YAQTSVKH | |
| BhCas12b | MATRSFILKIEPNEEVKKGLWKTHEVLNHGIAYYMNILKLIRQEAIYEHHEQDPKNPKKVS | SEQ ID NO: 42 |
| B. hisashii | KAEIQAELWDFVLKMQKCNSFTHEVDKDEVFNILRELYEELVPSSVEKKGEANQLSNKFLY | |
| 1108 AA | PLVDPNSQSGKGTASSGRKPRWYNLKIAGDPSWEEEKKKWEEDKKKDPLAKILGKLAEYGL | |
| 130.4kDa | IPLFIPYTDSNEPIVKEIKWMEKSRNQSVRRLDKDMFIQALERFLSWESWNLKVKEEYEKV | |
| | EKEYKTLEERIKEDIQALKALEQYEKERQEQLLRDTLNTNEYRLSKRGLRGWREIIQKWLK | |
| | MDENEPSEKYLEVFKDYQRKHPREAGDYSVYEFLSKKENHFIWRNHPEYPYLYATFCEIDK | |
| | KKKDAKQQATFTLADPINHPLWVRFEERSGSNLNKYRILTEQLHTEKLKKKLTVQLDRLIY | |
| | PTESGGWEEKGKVDIVLLPSRQFYNQIFLDIEEKGKHAFTYKDESIKFPLKGTLGGARVQF | |
| | DRDHLRRYPHKVESGNVGRIYFNMTVNIEPTESPVSKSLKIHRDDFPKVVNFKPKELTEWI | |
| | KDSKGKKLKSGIESLEIGLRVMSIDLGQRQAAAASIFEVVDQKPDIEGKLFFPIKGTELYA | |
| | VHRASFNIKLPGETLVKSREVLRKAREDNLKLMNQKLNFLRNVLHFQQFEDITEREKRVTK | |
| | WISRQENSDVPLVYQDELIQIRELMYKPYKDWVAFLKQLHKRLEVEIGKEVKHWRKSLSDG | |
| | RKGLYGISLKNIDEIDRTRKFLLRWSLRPTEPGEVRRLEPGQRFAIDQLNHLNALKEDRLK | |
| | KMANTIIMHALGYCYDVRKKKWQAKNPACQIILFEDLSNYNPYEERSRFENSKLMKWSRRE | |
| | IPRQVALQGEIYGLQVGEVGAQFSSRFHAKTGSPGIRCSVVTKEKLQDNRFFKNLQREGRL | |
| | TLDKIAVLKEGDLYPDKGGEKFISLSKDRKCVTTHADINAAQNLQKRFWTRTHGFYKVYCK | |
| | AYQVDGQTVYIPESKDQKQKIIEEFGEGYFILKDGVYEWVNAGKLKIKKGSSKQSSSELVD | |
| | SDILKDSFDLASELKGEKLMLYRDPSGNVFPSDKWMAAGVFFGKLERILISKLTNQYSIST | |
| | IEDDSSKQSM | |

Additional exemplary Cas9 equivalent protein sequences can include the following:

| Description | Sequence |
|---|---|
| AsCas12a | MTQFEGFTNLYQVSKTLRFELIPQGKTLKHIQEQGFIEEDKARNDHYKELKPIIDRIYKT |
| (previously | YADQCLQLVQLDWENLSAAIDSYRKEKTEETRNALIEEQATYRNAIHDYFIGRTDNLTDA |
| known as | INKRHAEIYKGLFKAELFNGKVLKQLGTVTTTEHENALLRSFDKFTTYFSGFYENRKNVF |
| Cpf1) | SAEDISTAIPHRIVQDNFPKFKENCHIFTRLITAVPSLREHFENVKKAIGIFVSTSIEEV |
| Acidaminococcus | FSFPFYNQLLTQTQIDLYNQLLGGISREAGTEKIKGLNEVLNLAIQKNDETAHIIASLPH |
| sp. | RFIPLFKQILSDRNTLSFILEEFKSDEEVIQSFCKYKTLLRNENVLETAEALFNELNSID |
| (strain | LTHIFISHKKLETISSALCDHWDTLRNALYERRISELTGKITKSAKEKVQRSLKHEDINL |
| BV3L6) | QEIISAAGKELSEAFKQKTSEILSHAHAALDQPLPTTLKKQEEKEILKSQLDSLLGLYHL |
| UniProtKB | LDWFAVDESNEVDPEFSARLTGIKLEMEPSLSFYNKARNYATKKPYSVEKFKLNFQMPTL |
| U2UMQ6 | ASGWDVNKEKNNGAILFVKNGLYYLGIMPKQKGRYKALSFEPTEKTSEGFDKMYYDYFPD |
| | AAKMIPKCSTQLKAVTAHFQTHTTPILLSNNFIEPLEITKEIYDLNNPEKEPKKFQTAYA |
| | KKTGDQKGYREALCKWIDFTRDFLSKYTKTTSIDLSSLRPSSQYKDLGEYYAELNPLLYH |
| | ISFQRIAEKEIMDAVETGKLYLFQIYNKDFAKGHHGKPNLHTLYWTGLFSPENLAKTSIK |
| | LNGQAELFYRPKSRMKRMAHRLGEKMLNKKLKDQKTPIPDTLYQELYDYVNHRLSHDLSD |
| | EARALLPNVITKEVSHEIIKDRRFTSDKFFFHVPITLNYQAANSPSKFNQRVNAYLKEHP |
| | ETPIIGIDRGERNLIYITVIDSTGKILEQRSLNTIQQFDYQKKLDNREKERVAARQAWSV |
| | VGTIKDLKQGYLSQVIHEIVDLMIHYQAVWLENLNFGFKSKRTGIAEKAVYQQFEKMLI |
| | DKLNCLVLKDYPAEKVGGVLNPYQLTDQFTSFAKMGTQSGFLFYVPAPYTSKIDPLTGFV |
| | DPFVWKTIKNHESRKHFLEGFDFLHYDVKTGDFILHFKMNRNLSFQRGLPGFMPAWDIVF |
| | EKNETQFDAKGTPFIAGKRIVPVIENHRFTGRYRDLYPANELIALLEEKGIVFRDGSNIL |
| | PKLLENDDSHAIDTMVALIRSVLQMRNSNAATGEDYINSPVRDLNGVCFDSRFQNPEWPM |
| | DADANGAYHIALKGQLLLNHLKESKDLKLQNGISNQDWLAYIQELRN (SEQ ID NO: 43) |
| AsCas12a | MTQFEGFTNLYQVSKTLRFELIPQGKTLKHIQEQGFIEEDKARNDHYKELKPIIDRIYKT |
| nickase | YADQCLQLVQLDWENLSAAIDSYRKEKTEETRNALIEEQATYRNAIHDYFIGRTDNLTDA |
| (e.g., | INKRHAEIYKGLFKAELFNGKVLKQLGTVTTTEHENALLRSFDKFTTYFSGFYENRKNVF |
| R1226A) | SAEDISTAIPHRIVQDNFPKFKENCHIFTRLITAVPSLREHFENVKKAIGIFVSTSIEEV |
| | FSFPFYNQLLTQTQIDLYNQLLGGISREAGTEKIKGLNEVLNLAIQKNDETAHIIASLPH |
| | RFIPLFKQILSDRNTLSFILEEFKSDEEVIQSFCKYKTLLRNENVLETAEALFNELNSID |
| | LTHIFISHKKLETISSALCDHWDTLRNALYERRISELTGKITKSAKEKVQRSLKHEDINL |
| | QEIISAAGKELSEAFKQKTSEILSHAHAALDQPLPTTLKKQEEKEILKSQLDSLLGLYHL |
| | LDWFAVDESNEVDPEFSARLTGIKLEMEPSLSFYNKARNYATKKPYSVEKFKLNFQMPTL |
| | ASGWDVNKEKNNGAILFVKNGLYYLGIMPKQKGRYKALSFEPTEKTSEGFDKMYYDYFPD |
| | AAKMIPKCSTQLKAVTAHFQTHTTPILLSNNFIEPLEITKEIYDLNNPEKEPKKFQTAYA |
| | KKTGDQKGYREALCKWIDFTRDFLSKYTKTTSIDLSSLRPSSQYKDLGEYYAELNPLLYH |
| | ISFQRIAEKEIMDAVETGKLYLFQIYNKDFAKGHHGKPNLHTLYWTGLFSPENLAKTSIK |
| | LNGQAELFYRPKSRMKRMAHRLGEKMLNKKLKDQKTPIPDTLYQELYDYVNHRLSHDLSD |
| | EARALLPNVITKEVSHEIIKDRRFTSDKFFFHVPITLNYQAANSPSKFNQRVNAYLKEHP |
| | ETPIIGIDRGERNLIYITVIDSTGKILEQRSLNTIQQFDYQKKLDNREKERVAARQAWSV |
| | VGTIKDLKQGYLSQVIHEIVDLMIHYQAVWLENLNFGFKSKRTGIAEKAVYQQFEKMLI |
| | DKLNCLVLKDYPAEKVGGVLNPYQLTDQFTSFAKMGTQSGFLFYVPAPYTSKIDPLTGFV |
| | DPFVWKTIKNHESRKHFLEGFDFLHYDVKTGDFILHFKMNRNLSFQRGLPGFMPAWDIVF |
| | EKNETQFDAKGTPFIAGKRIVPVIENHRFTGRYRDLYPANELIALLEEKGIVFRDGSNIL |
| | PKLLENDDSHAIDTMVALIRSVLQMANSNAATGEDYINSPVRDLNGVCFDSRFQNPEWPM |
| | DADANGAYHIALKGQLLLNHLKESKDLKLQNGISNQDWLAYIQELRN (SEQ ID NO: 44) |
| LbCas12a | 1 MNYKTGLEDF IGKESLSKTL RNALIPTEST KIHMEEMGVI RDDELRAEKQ QELKEIMDDY |
| (previously | 61 YRTFIEEKLG QIQGIQWNSL FQKMEETMED ISVRKDLDKI QNEKRKEICC YFTSDKRFKD |
| known as | 121 LFNAKLITDI LPNFIKDNKE YTEEEKAEKE QTRVLFQRFA TAFTNYFNQR RNNFSEDNIS |
| Cpf1) | 181 TAISFRIVNE NSEIHLQNMR AFQRIEQQYP EEVCGMEEEY KDMLQEWQMK HIYSVDFYDR |
| Lachnospiraceae | 241 ELTQPGIEYY NGICGKINEH MNQFCQKNRI NKNDFRMKKL HKQILCKKSS YYEIPFRFES |
| bacterium | 301 DQEVYDALNE FIKTMKKKEI IRRCVHLGQE CDDYDLGKIY ISSNKYEQIS NALYGSWDTI |
| GAM79 | 361 RKCIKEEYMD ALPGKGEKKE EKAEAAAKKE EYRSIADIDK IISLYGSEMD RTISAKKCIT |
| Ref Seq. | 421 EICDMAGQIS IDPLVCNSDI KLLQNKEKTT EIKTILDSFL HVYQWGQTFI VSDIIEKDSY |
| WP_119623382.1 | 481 FYSELEDVLE DFEGITTLYN HVRSYVTQKP YSTVKFKLHF GSPTLANGWS QSKEYDNNAI |
| | 541 LLMRDQKFYL GIFNVRNKPD KQIIKGHEKE EKGDYKKMIY NLLPGPSKML PKVFITSRSG |
| | 601 QETYKPSKHI LDGYNEKRHI KSSPKFDLGY CWDLIDYYKE CIHKHPDWKN YDFHFSDTKD |
| | 661 YEDISGFYRE VEMQGYQIKW TYISADEIQK LDEKGQIFLF QIYNKDFSVH STGKDNLHTM |
| | 721 YLKNLFSEEN LKDIVLKLNG EAELFFRKAS IKTPIVHKKG SVLVNRSYTQ TVGNKEIRVS |
| | 781 IPEEYYTEIY NYLNHIGKGK LSSEAQRYLD EGKIKSFTAT KDIVKNYRYC CDHYFLHLPI |
| | 841 TINFKAKSDV AVNERTLAYI AKKEDIHIIG IDRGERNLLY ISVVDVHGNI REQRSFNIVN |
| | 901 GYDYQQKLKD REKSRDAARK NWEEIEKIKE LKEGYLSMVI HYIAQLVVKY NAVVAMEDLN |
| | 961 YGFKTGRFKV ERQVYQKFET MLIEKLHYLV FKDREVCEEG GVLRGYQLTY IPESLKKVGK |
| | 1021 QCGFIFYVPA GYTSKIDPTT GFVNLFSFKN LTNRESRQDF VGKFDEIRYD RDKKMFEFSF |
| | 1081 DYNNYIKKGT ILASTKWKVY TNGTRLKRIV VNGKYTSQSM EVELTDAMEK MLQRAGIEYH |
| | 1141 DGKDLKGQIV EKGIEAEIID IFRLTVQMRN SRSESEDREY DRLISPVLND KGEFFDTATA |
| | 1201 DKTLPQDADA NGAYCIALKG LYEVKQIKEN WKENEQFPRN KLVQDNKTWF DFMQKKRYL |
| | (SEQ ID NO: 45) |
| PcCas12a - | 1 MAKNFEDFKR LYSLSKTLRF EAKPIGATLD NIVKSGLLDE DEHRAASYVK VKKLIDEYHK |
| previously | 61 VFIDRVLDDG CLPLENKGNN NSLAEYYESY VSRAQDEDAK KKFKEIQQNL RSVIAKKLTE |
| known at Cpf1 | 121 DKAYANLFGN KLIESYKDKE DKKKIIDSDL IQFINTAEST QLDSMSQDEA KELVKEFWGF |
| Prevotella. | 181 VTYFYGFFDN RKNMYTAEEK STGIAYRLVN ENLPKFIDNI EAFNRAITRP EIQENMGVLY |
| copri | 241 SDFSEYLNVE SIQEMFQLDY YNMLLTQKQI DVYNAIIGGK TDDEHDVKIK GINEYINLYN |
| Ref Seq. | 301 QQHKDDKLPK LKALFKQILS DRNAISWLPE EFNSDQEVLN AIKDCYERLA ENVLGDKVLK |
| WP_119227726. | 361 SLLGSLADYS LDGIFIRNDL QLTDISQKMF GNWGVIQNAI MQNIKRVAPA RKHKESEEDY |
| 1 | 421 EKRIAGIFKK ADSFSISYIN DCLNEADPNN AYFVENYFAT FGAVNTPTMQ RENLFALVQN |
| | 481 AYTEVAALLH SDYPTVKHLA QDKANVSKIK ALLDAIKSLQ HFVKPLLGKG DESDKDERFY |
| | 541 GELASLWAEL DTVTPLYNMI RNYMTRKPYS QKKIKLNFEN PQLLGGWDAN KEKDYATIIL |
| | 601 RRNGLYYLAI MDKDSRKLLG KAMPSDGECY EKMVYKFFKD VTTMIPKCST QLKDVQAYFK |
| | 661 VNTDDYVLNS KAFNKPLTIT KEVFDLNNVL YGKYKKFQKG YLTATGDNVG YTHAVNVWIK |
| | 721 FCMDFLNSYD STCIYDFSSL KPESYLSLDA FYQDANLLLY KLSFARASVS YINQLVEEGK |
| | 781 MYLFQIYNKD FSEYSKGTPN MHTLYWKALF DERNLADVVY KLNGQAEMFY RKKSIENTHP |
| | 841 THPANHPILN KNKDNKKKES LFDYDLIKDR RYTVDKFMFH VPITMNFKSV GSENINQDVK |
| | 901 AYLRHADDMH IIGIDRGERH LLYLVVIDLQ GNIKEQYSLN EIVNEYNGNT YHTNYHDLLD |
| | 961 VREEERLKAR QSWQTIENIK ELKEGYLSQV IHKITQLMVR YHAIVVLEDL SKGFMRSRQK |
| | 1021 VEKQVYQKFE KMLIDKLNYL VDKKTDVSTP GGLLNAYQLT CKSDSSQKLG KQSGFLFYIP |
| | 1081 AWNTSKIDPV TGFVNLLDTH SLNSKEKIKA FFSKFDAIRY NKDKKWFEFN LDYDKFGKKA |
| | 1141 EDTRTKWTLC TRGMRIDTFR NKEKNSQWDN QEVDLTTEMK SLLEHYYIDI HGNLKDAISA |
| | 1201 QTDKAFFTGL LHILKLTLQM RNSITGTETD YLVSPVADEN GIFYDSRSCG NQLPENADAN |
| | 1261 GAYNIARKGL MLIEQIKNAE DLNNVKFDIS NKAWLNFAQQ KPYKNG |
| | (SEQ ID NO: 46) |
| ErCas12a - | 1 MFSAKLISDI LPEFVIHNNN YSASEKEEKT QVIKLFSRFA TSFKDYFKNR ANCFSANDIS |
| previously | 61 SSSCHRIVND NAEIFFSNAL VYRRIVKNLS NDDINKISGD MKDSLKEMSL EEIYSYEKYG |
| known at Cpf1 | 121 EFITQEGISF YNDICGKVNL FMNLYCQKNK ENKNLYKLRK LHKQILCIAD TSYEVPYKFE |
| Eubacterium | 181 SDEEVYQSVN GFLDNISSKH IVERLRKIGE NYNGYNLDKI YIVSKFYESV SQKTYRDWET |
| rectale | 241 INTALEIHYN NILPGNGKSK ADKVKKAVKN DLQKSITEIN ELVSNYKLCP DDNIKAETYI |
| Ref Seq. | 301 HEISHILNNF EAQELKYNPE IHLVESELKA SELKNVLDVI MNAFHWCSVF MTEELVDKDN |
| WP_119223642. | 361 NFYAELEEIY DEIYPVISLY NLVRNYVTQK PYSTKKIKLN FGIPTLADGW SKSKEYSNNA |
| 1 | 421 IILMRDNLYY LGIFNAKNKP DKKIIEGNTS ENKGDYKKMI YNLLPGPNKM IPKVFLSSKT |
| | 481 GVETYKPSAY ILEGYKQNKH LKSSKDFDIT FCHDLIDYFK NCIAIHPEWK NFGFDFSDTS |
| | 541 TYEDISGFYR EVELQGYKID WTYISEKDID LLQEKGQLYL FQIYNKDFSK KSSGNDNLHT |
| | 601 MYLKNLFSEE NLKDIVLKLN GEAEIFFRKS SIKNPIIHKK GSILVNRTYE AEEKDQFGNI |
| | 661 QIVRKTIPEN IYQELYKYFN DKSDKELSDE AAKLKNVVGH HEAATNIVKD YRYTYDKYFL |
| | 721 HMPITINFKA NKTSFINDRI LQYIAKEKDL HVIGIDRGER NLIYVSVIDT CGNIVEQKSF |
| | 781 NIVNGYDYQI KLKQQEGARQ IARKEWKEIG KIKEIKEGYL SLVIHEISKM VIKYNAIIAM |
| | 841 EDLSYGFKKG RFKVERQVYQ KFETMLINKL NYLVFKDISI TENGGLLKGY QLTYIPDKLK |
| | 901 NVGHQCGCIF YVPAAYTSKI DPTTGFVNIF KFKDLTVDAK REFIKKFDSI RYDSDKNLFC |
| | 961 FTFDYNNFIT QNTVMSKSSW SVYTYGVRIK RRFVNGRFSN ESDTIDITKD MEKTLEMTDI |
| | 1021 NWRDGHDLRQ DIIDYEIVQH IFEIFKLTVQ MRNSLSELED RDYDRLISPV LNENNIFYDS |
| | 1081 AKAGDALPKD ADANGAYCIA LKGLYEIKQI TENWKEDGKF SRDKLKISNK DWFDFIQNKR |
| | 1141 YL (SEQ ID NO: 47) |
| CsCas12a - | 1 MNYKTGLEDF IGKESLSKTL RNALIPTEST KIHMEEMGVI RDDELRAEKQ QELKEIMDDY |
| previously | 61 YRAFIEEKLG QIQGIQWNSL FQKMEETMED ISVRKDLDKI QNEKRKEICC YFTSDKRFKD |
| known at Cpf1 | 121 LFNAKLITDI LPNFIKDNKE YTEEEKAEKE QTRVLFQRFA TAFTNYFNQR RNNFSEDNIS |
| Clostridium | 181 TAISFRIVNE NSEIHLQNMR AFQRIEQQYP EEVCGMEEEY KDMLQEWQMK HIYLVDFYDR |
| sp. AF34-10BH | 241 VLTQPGIEYY NGICGKINEH MNQFCQKNRI NKNDFRMKKL HKQILCKKSS YYEIPFRFES |
| Ref Seq. | 301 DQEVYDALNE FIKTMKEKEI ICRCVHLGQK CDDYDLGKIY ISSNKYEQIS NALYGSWDTI |
| WP_118538418. | 361 RKCIKEEYMD ALPGKGEKKE EKAEAAAKKE EYRSIADIDK IISLYGSEMD RTISAKKCIT |
| 1 | 421 EICDMAGQIS TDPLVCNSDI KLLQNKEKTT EIKTILDSFL HVYQWGQTFI VSDIIEKDSY |
| | 481 FYSELEDVLE DFEGITTLYN HVRSYVTQKP YSTVKFKLHF GSPTLANGWS QSKEYDNNAI |
| | 541 LLMRDQKFYL GIFNVRNKPD KQIIKGHEKE EKGDYKKMIY NLLPGPSKML PKVFITSRSG |
| | 601 QETYKPSKHI LDGYNEKRHI KSSPKFDLGY CWDLIDYYKE CIHKHPDWKN YDFHFSDTKD |
| | 661 YEDISGFYRE VEMQGYQIKW TYISADEIQK LDEKGQIFLF QIYNKDFSVH STGKDNLHTM |
| | 721 YLKNLFSEEN LKDIVLKLNG EAELFFRKAS IKTPIVHKKG SVLVNRSYTQ TVGNKEIRVS |
| | 781 IPEEYYTEIY NYLNHIGRGK LSTEAQRYLE ERKIKSFTAT KDIVKNYRYC CDHYFLHLPI |
| | 841 TINFKAKSDI AVNERTLAYI AKKEDIHIIG IDRGERNLLY ISVVDVHGNI REQRSFNIVN |
| | 901 GYDYQQKLKD REKSRDAARK NWEEIEKIKE LKEGYLSMVI HYIAQLVVKY NAVVAMEDLN |
| | 961 YGFKTGRFKV ERQVYQKFET MLIEKLHYLV FKDREVCEEG GVLRGYQLTY IPESLKKVGK |
| | 1021 QCGFIFYVPA GYTSKIDPTT GFVNLFSFKN LTNRESRQDF VGKFDEIRYD RDKKMFEFSF |
| | 1081 DYNNYIKKGT MLASTKWKVY TNGTRLKRIV VNGKYTSQSM EVELTDAMEK MLQRAGIEYH |
| | 1141 DGKDLKGQIV EKGIEAEIID IFRLTVQMRN SRSESEDREY DRLISPVLND KGEFFDTATA |
| | 1201 DKTLPQDADA NGAYCIALKG LYEVKQIKEN WKENEQFPRN KLVQDNKTWF DFMQKKRYL |
| | (SEQ ID NO: 48) |
| BhCas12b | 1 MATRSFILKI EPNEEVKKGL WKTHEVLNHG IAYYMNILKL IRQEAIYEHH EQDPKNPKKV |
| Bacillus | 61 SKAEIQAELW DFVLKMQKCN SFTHEVDKDE VFNILRELYE ELVPSSVEKK GEANQLSNKF |
| hisashii | 121 LYPLVDPNSQ SGKGTASSGR KPRWYNLKIA GDPSWEEEKK KWEEDKKKDP LAKILGKLAE |
| Ref Seq. | 181 YGLIPLFIPY TDSNEPIVKE IKWMEKSRNQ SVRRLDKDMF IQALERFLSW ESWNLKVKEE |
| WP_095142515. | 241 YEKVEKEYKT LEERIKEDIQ ALKALEQYEK ERQEQLLRDT LNTNEYRLSK RGLRGWREII |
| 1 | 301 QKWLKMDENE PSEKYLEVFK DYQRKHPREA GDYSVYEFLS KKENHFIWRN HPEYPYLYAT |
| | 361 FCEIDKKKKD AKQQATFTLA DPINHPLWVR FEERSGSNLN KYRILTEQLH TEKLKKKLTV |
| | 421 QLDRLIYPTE SGGWEEKGKV DIVLLPSRQF YNQIFLDIEE KGKHAFTYKD ESIKFPLKGT |
| | 481 LGGARVQFDR DHLRRYPHKV ESGNVGRIYF NMTVNIEPTE SPVSKSLKIH RDDFPKVVNF |
| | 541 KPKELTEWIK DSKGKKLKSG IESLEIGLRV MSIDLGQRQA AAASIFEVVD QKPDIEGKLF |
| | 601 FPIKGTELYA VHRASFNIKL PGETLVKSRE VLRKAREDNL KLMNQKLNFL RNVLHFQQFE |
| | 661 DITEREKRVT KWISRQENSD VPLVYQDELI QIRELMYKPY KDWVAFLKQL HKRLEVEIGK |
| | 721 EVKHWRKSLS DGRKGLYGIS LKNIDEIDRT RKFLLRWSLR PTEPGEVRRL EPGQRFAIDQ |
| | 781 LNHLNALKED RLKKMANTII MHALGYCYDV RKKKWQAKNP ACQIILFEDL SNYNPYEERS |
| | 841 RFENSKLMKW SRREIPRQVA LQGEIYGLQV GEVGAQFSSR FHAKTGSPGI RCSVVTKEKL |
| | 901 QDNRFFKNLQ REGRLTLDKI AVLKEGDLYP DKGGEKFISL SKDRKCVTTH ADINAAQNLQ |
| | 961 KRFWTRTHGF YKVYCKAYQV DGQTVYIPES KDQKQKIIEE FGEGYFILKD GVYEWVNAGK |
| | 1021 LKIKKGSSKQ SSSELVDSDI LKDSFDLASE LKGEKLMLYR DPSGNVFPSD KWMAAGVFFG |
| | 1081 KLERILISKL TNQYSISTIE DDSSKQSM (SEQ ID NO: 49) |
| ThCas12b | 1 MSEKTTQRAY TLRLNRASGE CAVCQNNSCD CWHDALWATH KAVNRGAKAF GDWLLTLRGG |
| Thermomonas | 61 LCHTLVEMEV PAKGNNPPQR PTDQERRDRR VLLALSWLSV EDEHGAPKEF IVATGRDSAD |
| hydrothermalis | 121 DRAKKVEEKL REILEKRDFQ EHEIDAWLQD CGPSLKAHIR EDAVWVNRRA LFDAAVERIK |
| Ref Seq. | 181 TLTWEEAWDF LEPFFGTQYF AGIGDGKDKD DAEGPARQGE KAKDLVQKAG QWLSARFGIG |
| WP_072754838 | 241 TGADFMSMAE AYEKIAKWAS QAQNGDNGKA TIEKLACALR PSEPPTLDTV LKCISGPGHK |
| | 301 SATREYLKTL DKKSTVTQED LNQLRKLADE DARNCRKKVG KKGKKPWADE VLKDVENSCE |
| | 361 LTYLQDNSPA RHREFSVMLD HAARRVSMAH SWIKKAEQRR RQFESDAQKL KNLQERAPSA |
| | 421 VEWLDRFCES RSMTTGANTG SGYRIRKRAI EGWSYVVQAW AEASCDTEDK RIAAARKVQA |
| | 481 DPEIEKFGDI QLFEALAADE AICVWRDQEG TQNPSILIDY VTGKTAEHNQ KRFKVPAYRH |
| | 541 PDELRHPVFC DFGNSRWSIQ FAIHKEIRDR DKGAKQDTRQ LQNRHGLKMR LWNGRSMTDV |
| | 601 NLHWSSKRLT ADLALDQNPN PNPTEVTRAD RLGRAASSAF DHVKIKNVFN EKEWNGRLQA |
| | 661 PRAELDRIAK LEEQGKTEQA EKLRKRLRWY VSFSPCLSPS GPFIVYAGQH NIQPKRSGQY |
| | 721 APHAQANKGR ARLAQLILSR LPDLRILSVD LGHRFAAACA VWETLSSDAF RREIQGLNVL |
| | 781 AGGSGEGDLF LHVEMTGDDG KRRTVVYRRI GPDQLLDNTP HPAPWARLDR QFLIKLQGED |
| | 841 EGVREASNEE LWTVHKLEVE VGRTVPLIDR MVRSGFGKTE KQKERLKKLR ELGWISAMPN |
| | 901 EPSAETDEKE GEIRSISRSV DELMSSALGT LRLALKRHGN RARIAFAMTA DYKPMPGGQK |
| | 961 YYFHEAKEAS KNDDETKRRD NQIEFLQDAL SLWHDLFSSP DWEDNEAKKL WQNHIATLPN |
| | 1021 YQTPEEISAE LKRVERNKKR KENRDKLRTA AKALAENDQL RQHLHDTWKE RWESDDQQWK |
| | 1081 ERLRSLKDWI FPRGKAEDNP SIRHVGGLSI TRINTISGLY QILKAFKMRP EPDDLRKNIP |
| | 1141 QKGDDELENF NRRLLEARDR LREQRVKQLA SRIIEAALGV GRIKIPKNGK LPKRPRTTVD |
| | 1201 TPCHAVVIES LKTYRPDDLR TRRENRQLMQ WSSAKVRKYL KEGCELYGLH FLEVPANYTS |
| | 1261 RQCSRTGLPG IRCDDVPTGD FLKAPWWRRA INTAREKNGG DAKDRFLVDL YDHLNNLQSK |
| | 1321 GEALPATVRV PRQGGNLFIA GAQLDDTNKE RRAIQADLNA AANIGLRALL DPDWRGRWWY |
| | 1381 VPCKDGTSEP ALDRIEGSTA FNDVRSLPTG DNSSRRAPRE IENLWRDPSC |
| | 1441 |
| LsCas12b | 1 MSIRSFKLKL KTKSGVNAEQ LRRGLWRTHQ LINDGIAYYM NWLVLLRQED LFIRNKETNE |
| Laceyella | 61 IEKRSKEEIQ AVLLERVHKQ QQRNQWSGEV DEQTLLQALR QLYEEIVPSV IGKSGNASLK |
| sacchari | 121 ARFFLGPLVD PNNKTTKDVS KSGPTPKWKK MKDAGDPNWV QEYEKYMAER QTLVRLEEMG |
| WP_132221894. | 181 LIPLFPMYTD EVGDIHWLPQ ASGYTRTWDR DMFQQAIERL LSWESWNRRV RERRAQFEKK |
| 1 | 241 THDFASRFSE SDVQWMNKLR EYEAQQEKSL EENAFAPNEP YALTKKALRG WERVYHSWMR |
| | 301 LDSAASEEAY WQEVATCQTA MRGEFGDPAI YQFLAQKENH DIWRGYPERV IDFAELNHLQ |
| | 361 RELRRAKEDA TFTLPDSVDH PLWVRYEAPG GTNIHGYDLV QDTKRNLTLI LDKFILPDEN |
| | 421 GSWHEVKKVP FSLAKSKQFH RQVWLQEEQK QKKREVVFYD YSTNLPHLGT LAGAKLQWDR |
| | 481 NFLNKRTQQQ IEETGEIGKV FFNISVDVRP AVEVKNGRLQ NGLGKALTVL THPDGTKIVT |
| | 541 GWKAEQLEKW VGESGRVSSL GLDSLSEGLR VMSIDLGQRT SATVSVFEIT KEAPDNPYKF |
| | 601 FYQLEGTEMF AVHQRSFLLA LPGENPPQKI KQMREIRWKE RNRIKQQVDQ LSAILRLHKK |
| | 661 VNEDERIQAI DKLLQKVASW QLNEEIATAW NQALSQLYSK AKENDLQWNQ AIKNAHHQLE |
| | 721 PWGKQISLVV RKDLSTGRQG IAGLSLWSIE ELEATKKLLT RWSKRSREPG VVKRIERFET |
| | 781 FAKQIQHHIN QVKENRLKQL ANLIVMTALG YKYDQEQKKW IEVYPACQVV LFENLRSYRF |
| | 841 SFERSRRENK KLMEWSHRSI PKLVQMQGEL FGLQVADVYA AYSSRYHGRT GAPGIRCHAL |
| | 901 TEADLRNETN IIHELIEAGF IKEEHRPYLQ QGDLVPWSGG ELFATLQKPY DNPRILTLHA |
| | 961 DINAAQNIQK RFWHPSMWFR VNCESVMEGE IVTYVPKNKT VHKKQGKTFR FVKVEGSDVY |
| | 1021 EWAKWSKNRN KNTFSSITER KPPSSMILFR DPSGTFFKEQ EWVEQKTFWG KVQSMIQAYM |
| | 1081 KKTIVQRMEE (SEQ ID NO: 51) |
| DtCas12b | 1 MVLGRKDDTA ELRRALWTTH EHVNLAVAEV ERVLLRCRGR SYWTLDRRGD PVHVPESQVA |
| Dsulfonatronum | 61 EDALAMAREA QRRNGVVPWG EDEEILLALR YLYEQIVPSC LLDDLGKPLK GDAQKIGTNY |
| thiodismutans | 121 AGPLFDSDTC RRDEGKDVAC CGPFHEVAGK YLGALPEWAT PISKQEFDGK DASHLRFKAT |
| WP_031386437 | 181 GGDDAFFRVS IEKANAWYED PANQDALKNK AYNKDDWKKE KDKGISSWAV KYIQKQLQLG |
| | 241 QDPRTEVRRK LWLELGLLPL FIPVFDKTMV GNLWNRLAVR LALAHLLSWE SWNHRAVQDQ |
| | 301 ALARAKRDEL AALFLGMEDG FAGLREYELR RNESIKQHAF EPVDRPYWSG RALRSVVTRV |
| | 361 REEWLRHGDT QESRKNICNR LQDRLRGKFG DPDVFHWLAE DGQEALWKER DCVTSFSLLN |
| | 421 DADGLLEKRK GYALMTFADA RLHPRWAMYE APGGSNLRTY QIRKTENGLW ADVVLLSPRN |
| | 481 ESAAVEEKTF NVRLAPSGQL SNVSFDQIQK GSKMVGRCRY QSANQQFEGL LGGAEILFDR |
| | 541 KRIANEQHGA TDLASKPGHV WFKLTLDVRP QAPQGWLDGK GRPALPPEAK HFKTALSNKS |
| | 601 KFADQVRPGL RVLSVDLGVR SFAACSVFEL VRGGPDQGTY FPAADGRTVD DPEKLWAKHE |
| | 661 RSFKITLPGE NPSRKEEIAR RAAMEELRSL NGDIRRLKAI LRLSVLQEDD PRTEHLRLFM |
| | 721 EAIVDDPAKS ALNAELFKGF GDDRFRSTPD LWKQHCHFFH DKAEKVVAER FSRWRTETRP |
| | 781 KSSSWQDWRE RRGYAGGKSY WAVTYLEAVR GLILRWNMRG RTYGEVNRQD KKQFGTVASA |
| | 841 LLHHINQLKE DRIKTGADMI IQAARGFVPR KNGAGWVQVH EPCRLILFED LARYRFRTDR |
| | 901 SRRENSRLMR WSHREIVNEV GMQGELYGLH VDTTEAGFSS RYLASSGAPG VRCRHLVEED |
| | 961 FHDGLPGMHL VGELDWLLPK DKDRTANEAR RLLGGMVRPG MLVPWDGGEL FATLNAASQL |
| | 1021 HVIHADINAA QNLQRRFWGR CGEAIRIVCN QLSVDGSTRY EMAKAPKARL LGALQQLKNG |
| | 1081 DAPFHLTSIP NSQKPENSYV MTPTNAGKKY RAGPGEKSSG EEDELALDIV EQAEELAQGR |
| | 1141 KTFFRDPSGV FFAPDRWLPS EIYWSRIRRR IWQVTLERNS SGRQERAEMD EMPY |
| | (SEQ ID NO: 52) |

napDNAbps that Recognize Non-Canonical PAM Sequences

In some embodiments, the napDNAbp is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 2016 July; 34(7):768-73. PubMed PMID: 27136078; Swarts et al., Nature. 507(7491) (2014):258-61; and Swarts et al., Nucleic Acids Res. 43(10) (2015):5120-9, each of which is incorporated herein by reference.

In some embodiments, the disclosure provides napDNAbp domains that comprise SpCas9 variants that recognize and work best with NRRH, NRCH, and NRTH PAMs. See International Application No. PCT/US2019/47996, which published as International Publication No. WO 2020/041751 on Feb. 27, 2020, incorporated by reference herein. In some embodiments, the disclosed base editors comprise a napDNAbp domain selected from SpCas9-NRRH, SpCas9-NRTH, and SpCas9-NRCH.

In some embodiments, the disclosed base editors comprise a napDNAbp domain that has a sequence that is at least 90%, at least 95%, at least 98%, or at least 99% identical to SpCas9-NRRH. In some embodiments, the disclosed base editors comprise a napDNAbp domain that comprises SpCas9-NRRH. The SpCas9-NRRH has an amino acid sequence as presented in SEQ ID NO: 53 (underlined residues are mutated relative to SpCas9, as set forth in SEQ ID NO: 213).

(SEQ ID NO: 53)
MDKKYSIGLDIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMVKRYDEHHQDLTLLKALVRQQLPEKYKEI
FFDQSKNGYAGYIDGGASQEEFYKFIKPILEKMDG
TEELLVKLNREDLLRKQRTFDNGIIPHQIHLGELH
AILRRQGDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRLRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGGHKPENIVIEMARENQTT
QKGQKNSRERMKRIEEGIKELGSQILKEHPVENTQ
LQNEKLYLYYLQNGRDMYVDQELDINRLSDYDVDH
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKGNSDKLI
ARKKDWDPKKYGGFNSPTAAYSVLVVAKVEKGKSK
KLKSVKELLGITIMERSSFEKNPIGFLEAKGYKEV
KKDLIIKLPKYSLFELENGRKRMLASAGVLHKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGVPAAFKYFDTT
IDKKRYTSTKEVLDATLIHQSITGLYETRIDLSQL
GGD.

In some embodiments, the disclosed base editors comprise a napDNAbp domain that has a sequence that is at least 90%, at least 95%, at least 98%, or at least 99% identical to SpCas9-NRCH. In some embodiments, the disclosed base editors comprise a napDNAbp domain that comprises SpCas9-NRCH. The SpCas9-NRCH has an amino acid sequence as presented in SEQ ID NO: 54 (underlined residues are mutated relative to SpCas9)

(SEQ ID NO: 54)
MDKKYSIGLDIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMVKRYDEHHQDLTLLKALVRQQLPEKYKEI
FFDQSKNGYAGYIDGGASQEEFYKFIKPILEKMDG
TEELLVKLNREDLLRKQRTFDNGIIPHQIHLGELH
AILRRQGDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRLRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGGHKPENIVIEMARENQTT
QKGQKNSRERMKRIEEGIKELGSQILKEHPVENTQ
LQNEKLYLYYLQNGRDMYVDQELDINRLSDYDVDH
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKGNSDKLI
ARKKDWDPKKYGGFNSPTVAYSVLVVAKVEKGKSK
KLKSVKELLGITIMERSSFEKNPIDFLEAKGYKEV
KKDLIIKLPKYSLFELENGRKRMLASAGVLQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPAAFKYFDTT
INRKQYNTTKEVLDATLIRQSITGLYETRIDLSQL
GGD

In some embodiments, the disclosed base editors comprise a napDNAbp domain that has a sequence that is at least 90%, at least 95%, at least 98%, or at least 99% identical to SpCas9-NRTH. In some embodiments, the disclosed base editors comprise a napDNAbp domain that comprises SpCas9-NRTH. The SpCas9-NRTH has an amino acid sequence as presented in SEQ ID NO: 55 (underlined residues are mutated relative to SpCas9)

(SEQ ID NO: 55)
MDKKYSIGLDIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMVKRYDEHHQDLTLLKALVRQQLPEKYKEI
FFDQSKNGYAGYIDGGASQEEFYKFIKPILEKMDG
TEELLVKLNREDLLRKQRTFDNGIIPHQIHLGELH
AILRRQGDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRLRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGGHKPENIVIEMARENQTT
QKGQKNSRERMKRIEEGIKELGSQILKEHPVENTQ
LQNEKLYLYYLQNGRDMYVDQELDINRLSDYDVDH
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKGNSDKLI
ARKKDWDPKKYGGFNSPTVAYSVLVVAKVEKGKSK
KLKSVKELLGITIMERSSFEKNPIGFLEAKGYKEV
KKDLIIKLPKYSLFELENGRKRMLASASVLHKGNE
LALPSKYVNFLYLASHYEKLKGSSEDNKQKQLFVE
QHKHYLDEIIEQISEFSKRVILADANLDKVLSAYN
KHRDKPIREQAENIIHLFTLTNLGASAAFKYFDTT
IGRKLYTSTKEVLDATLIHQSITGLYETRIDLSQL
GGD

In other embodiments, the napDNAbp of any of the disclosed base editors comprises a Cas9 derived from a Streptococcus macacae, e.g. Streptococcus macacae NCTC 11558, or SmacCas9, or a variant thereof. In some embodiments, the napDNAbp comprises a hybrid variant of SmacCas9 that incorporates an SpCas9 domain with the SmacCas9 domain and is known as Spy-macCas9, or a variant thereof. In some embodiments, the napDNAbp comprises a hybrid variant of SmacCas9 that incorporates an increased nucleolytic variant of an SpCas9 (iSpy Cas9) domain and is known as iSpy-macCas9. Relative to Spymac-Cas9, iSpyMac-Cas9 contains two mutations, R221K and N394K, that were identified by deep mutational scans of Spy Cas9 that raise modification rates of the protein on most targets. See Jakimo et al., bioRxiv, A Cas9 with Complete PAM Recognition for Adenine Dinucleotides (September 2018), herein incorporated by reference. Jakimo et al. showed that the hybrids Spy-macCas9 and iSpy-macCas9 recognize a short 5′-NAA-3′ PAM and recognized all evaluated adenine dinucleotide PAM sequences and posseseds robust editing efficiency in human cells. Liu et al. engineered base editors containing Spy-mac Cas9, and demonstrated that cytidine and base editors containing Spymac domains can induce efficient C-to-T and A-to-G conversions in vivo. In addition, Liu et al. suggested that the PAM scope of Spy-mac Cas9 may be 5′-TAAA-3′, rather than 5′-NAA-3′ as reported by Jakimo et al. See Liu et al. Cell Discovery (2019) 5:58, herein incorporated by reference.

In some embodiments, the disclosed base editors comprise a napDNAbp domain that has a sequence that is at least 90%, at least 95%, at least 98%, or at least 99% identical to iSpyMac-Cas9. In some embodiments, the disclosed base editors comprise a napDNAbp domain that comprises iSpyMac-Cas9. The iSpyMac-Cas9 has an amino acid sequence as presented in SEQ ID NO: 56 (R221K and N394K mutations are underlined):

(SEQ ID NO: 56)
DKKYSIGLDIGTNSVGWAVITDEYKVPSKKFKVLG
NTDRHSIKKNLIGALLFDSGETAEATRLKRTARRR
YTRRKNRICYLQEIFSNEMAKVDDSFFHRLEESFL
VEEDKKHERHPIFGNIVDEVAYHEKYPTIYHLRKK
LVDSTDKADLRLIYLALAHMIKFRGHFLIEGDLNP
DNSDVDKLFIQLVQTYNQLFEENPINASGVDAKAI
LSARLSKSRKLENLIAQLPGEKKNGLFGNLIALSL
GLTPNFKSNFDLAEDAKLQLSKDTYDDDLDNLLAQ
IGDQYADLFLAAKNLSDAILLSDILRVNTEITKAP
LSASMIKRYDEHHQDLTLLKALVRQQLPEKYKEIF
FDQSKNGYAGYIDGGASQEEFYKFIKPILEKMDGT
EELLVKLKREDLLRKQRTFDNGSIPHQIHLGELHA
ILRRQEDFYPFLKDNREKIEKILTFRIPYYVGPLA
RGNSRFAWMTRKSEETITPWNFEEVVDKGASAQSF
IERMTNFDKNLPNEKVLPKHSLLYEYFTVYNELTK
VKYVTEGMRKPAFLSGEQKKAIVDLLFKTNRKVTV
KQLKEDYFKKIECFDSVEISGVEDRFNASLGTYHD
LLKIIKDKDFLDNEENEDILEDIVLTLTLFEDREM
IEERLKTYAHLFDDKVMKQLKRRRYTGWGRLSRKL
INGIRDKQSGKTILDFLKSDGFANRNFMQLIHDDS
LTFKEDIQKAQVSGQGDSLHEHIANLAGSPAIKKG
ILQTVKVVDELVKVMGRHKPENIVIEMARENQTTQ
KGQKNSRERMKRIEEGIKELGSQILKEHPVENTQL
QNEKLYLYYLQNGRDMYVDQELDINRLSDYDVDHI
VPQSFLKDDSIDNKVLTRSDKNRGKSDNVPSEEVV
KKMKNYWRQLLNAKLITQRKFDNLTKAERGGLSEL
DKAGFIKRQLVETRQITKHVAQILDSRMNTKYDEN
DKLIREVKVITLKSKLVSDFRKDFQFYKVREINNY
HHAHDAYLNAVVGTALIKKYPKLESEFVYGDYKVY
DVRKMIAKSEQEIGKATAKYFFYSNIMNFFKTEIT
LANGEIRKRPLIETNGETGEIVWDKGRDFATVRKV
LSMPQVNIVKKTEIQTVGQNGGLFDDNPKSPLEVT
PSKLVPLKKELNPKKYGGYQKPTTAYPVLLITDTK
QLIPISVMNKKQFEQNPVKFLRDRGYQQVGKNDFI
KLPKYTLVDIGDGIKRLWASSKEIHKGNQLVVSKK
SQILLYHAHHLDSDLSNDYLQNHNQQFDVLFNEII
SFSKKCKLGKEHIQKIENVYSNKKNSASIEELAES
FIKLLGFTQLGATSPFNFLGVKLNQKQYKGKKDYI
LPCTEGTLIRQSITGLYETRVDLSKIGED

In other embodiments, the napDNAbp of any of the disclosed base editors is a prokaryotic homolog of an Argonaute protein. Prokaryotic homologs of Argonaute proteins are known and have been described, for example, in Makarova K., et al., “Prokaryotic homologs of Argonaute proteins are predicted to function as key components of a novel system of defense against mobile genetic elements”, Biol Direct. 2009 Aug. 25; 4:29. doi: 10.1186/1745-6150-4-29, the entire contents of which is hereby incorporated by reference. In some embodiments, the napDNAbp is a Marinitoga piezophila Argunaute (MpAgo) protein. The CRISPR-associated Marinitoga piezophila Argunaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. See, e.g., Kaya et al., “A bacterial Argonaute with noncanonical guide RNA specificity”, Proc Natl Acad Sci USA. 2016 Apr. 12; 113(15):4057-62, the entire contents of which are hereby incorporated by reference). It should be appreciated that other argonaute proteins may be used, and are within the scope of this disclosure.

In some embodiments, the napDNAbp is a single effector of a microbial CRISPR-Cas system. Single effectors of microbial CRISPR-Cas systems include, without limitation, Cas9, Cpf1, C2c1, C2c2, and C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, C2c1 and C2c3, contain RuvC-like endonuclease domains related to Cpf1. A third system, C2c2 contains an effector with two predicated HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See, e.g., East-Seletsky, et al., “Two distinct RNase activities of CRISPR-C2c2 enable guide-RNA processing and RNA detection”, Nature, 2016 Oct. 13; 538(7624):270-273, the entire contents of which are hereby incorporated by reference. In vitro biochemical analysis of C2c2 in Leptotrichia shahii has shown that C2c2 is guided by a single CRISPR RNA and can be programed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector”, Science, 2016 Aug. 5; 353(6299), the entire contents of which are hereby incorporated by reference.

The crystal structure of Alicyclobaccillus acidoterrastris C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the napDNAbp may be a C2c1, a C2c2, or a C2c3 protein. In some embodiments, the napDNAbp is a C2c1 protein. In some embodiments, the napDNAbp is a C2c2 protein. In some embodiments, the napDNAbp is a C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring C2c1, C2c2, or C2c3 protein. In some embodiments, the napDNAbp is a naturally-occurring C2c1, C2c2, or C2c3 protein.

Some aspects of the disclosure provide Cas9 domains that have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base editing base editors provided herein may need to be placed at a precise location, for example where a target base is placed within a 4 base region (e.g., a “editing window” or a “target window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the base editors provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

For example, a napDNAbp domain with altered PAM specificity, such as a domain with at least 80%, at least 85%, at least 90%, at least 95%, or at least 99% sequence identity with wild type Francisella novicida Cpf1 (SEQ ID NO: 218) (D91′7, E1006, and D1255), which has the following amino acid sequence:

(SEQ ID NO: 218)
MSIYQEFVNKYSLSKTLRFELIPQGKTLENIKARG
LILDDEKRAKDYKKAKQIIDKYHQFFIEEILSSVC
ISEDLLQNYSDVYFKLKKSDDDNLQKDFKSAKDTI
KKQISEYIKDSEKFKNLFNQNLIDAKKGQESDLIL
WLKQSKDNGIELFKANSDITDIDEALEIIKSFKGW
TTYFKGFHENRKNVYSSNDIPTSIIYRIVDDNLPK
FLENKAKYESLKDKAPEAINYEQIKKDLAEELTFD
IDYKTSEVNQRVFSLDEVFEIANFNNYLNQSGITK
FNTIIGGKFVNGENTKRKGINEYINLYSQQINDKT
LKKYKMSVLFKQILSDTESKSFVIDKLEDDSDVVT
TMQSFYEQIAAFKTVEEKSIKETLSLLFDDLKAQK
LDLSKIYFKNDKSLTDLSQQVFDDYSVIGTAVLEY
ITQQIAPKNLDNPSKKEQELIAKKTEKAKYLSLET
IKLALEEFNKHRDIDKQCRFEEILANFAAIPMIFD
EIAQNKDNLAQISIKYQNQGKKDLLQASAEDDVKA
IKDLLDQTNNLLHKLKIFHISQSEDKANILDKDEH
FYLVFEECYFELANIVPLYNKIRNYITQKPYSDEK
FKLNFENSTLANGWDKNKEPDNTAILFIKDDKYYL
GVMNKKNNKIFDDKAIKENKGEGYKKIVYKLLPGA
NKMLPKVFFSAKSIKFYNPSEDILRIRNHSTHTKN
GSPQKGYEKFEFNIEDCRKFIDFYKQSISKHPEWK
DFGFRFSDTQRYNSIDEFYREVENQGYKLTFENIS
ESYIDSVVNQGKLYLFQIYNKDFSAYSKGRPNLHT
LYWKALFDERNLQDVVYKLNGEAELFYRKQSIPKK
ITHPAKEAIANKNKDNPKKESVFEYDLIKDKRFTE
DKFFFHCPITINFKSSGANKFNDEINLLLKEKAND
VHILSIDRGERHLAYYTLVDGKGNIIKQDTFNIIG
NDRMKTNYHDKLAAIEKDRDSARKDWKKINNIKEM
KEGYLSQVVHEIAKLVIEYNAIVVFEDLNFGFKRG
RFKVEKQVYQKLEKMLIEKLNYLVFKDNEFDKTGG
VLRAYQLTAPFETFKKMGKQTGIIYYVPAGFTSKI
CPVTGFVNQLYPKYESVSKSQEFFSKFDKICYNLD
KGYFEFSFDYKNFGDKAAKGKWTIASFGSRLINFR
NSDKNHNWDTREVYPTKELEKLLKDYSIEYGHGEC
IKAAICGESDKKFFAKLTSVLNTILQMRNSKTGTE
LDYLISPVADVNGNFFDSRQAPKNMPQDADANGAY
HIGLKGLMLLGRIKNNQEGKKLNLVIKNEEYFEFV
QNRNN

An additional napDNAbp domain with altered PAM specificity, such as a domain having at least 80%, at least 85%, at least 90%, at least 95%, or at least 99% sequence identity with wild type Geobacillus thermodenitrificans Cas9 (SEQ ID NO: 217), which has the following amino acid sequence:

(SEQ ID NO: 217)
MKYKIGLDIGITSIGWAVINLDIPRIEDLGVRIFD
RAENPKTGESLALPRRLARSARRRLRRRKHRLERI
RRLFVREGILTKEELNKLFEKKHEIDVWQLRVEAL
DRKLNNDELARILLHLAKRRGFRSNRKSERTNKEN
STMLKHIEENQSILSSYRTVAEMVVKDPKFSLHKR
NKEDNYTNTVARDDLEREIKLIFAKQREYGNIVCT
EAFEHEYISIWASQRPFASKDDIEKKVGFCTFEPK
EKRAPKATYTFQSFTVWEHINKLRLVSPGGIRALT
DDERRLIYKQAFHKNKITFHDVRTLLNLPDDTRFK
GLLYDRNTTLKENEKVRFLELGAYHKIRKAIDSVY
GKGAAKSFRPIDFDTFGYALTMFKDDTDIRSYLRN
EYEQNGKRMENLADKVYDEELIEELLNLSFSKFGH
LSLKALRNILPYMEQGEVYSTACERAGYTFTGPKK
KQKTVLLPNIPPIANPVVMRALTQARKVVNAIIKK
YGSPVSIHIELARELSQSFDERRKMQKEQEGNRKK
NETAIRQLVEYGLTLNPTGLDIVKFKLWSEQNGKC
AYSLQPIEIERLLEPGYTEVDHVIPYSRSLDDSYT
NKVLVLTKENREKGNRTPAEYLGLGSERWQQFETF
VLTNKQFSKKKRDRLLRLHYDENEENEFKNRNLND
TRYISRFLANFIREHLKFADSDDKQKVYTVNGRIT
AHLRSRWNFNKNREESNLHHAVDAAIVACTTPSDI
ARVTAFYQRREQNKELSKKTDPQFPQPWPHFADEL
QARLSKNPKESIKALNLGNYDNEKLESLQPVFVSR
MPKRSITGAAHQETLRRYIGIDERSGKIQTVVKKK
LSEIQLDKTGHFPMYGKESDPRTYEAIRQRLLEHN
NDPKKAFQEPLYKPKKNGELGPIIRTIKIIDTTNQ
VIPLNDGKTVAYNSNIVRVDVFEKDGKYYCVPIYT
IDMMKGILPNKAIEPNKPYSEWKEMTEDYTFRFSL
YPNDLIRIEFPREKTIKTAVGEEIKIKDLFAYYQT
IDSSNGGLSLVSHDNNFSLRSIGSRTLKRFEKYQV
DVLGNIYKVRGEKRVGVASSSHSKAGETIRPL

In some embodiments, the nucleic acid programmable DNA binding protein (napDNAbp) is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 34(7): 768-73 (2016), PubMed PMID: 27136078; Swarts et al., Nature, 507(7491): 258-61 (2014); and Swarts et al., Nucleic Acids Res. 43(10) (2015): 5120-9, each of which is incorporated herein by reference. The sequence of Natronobacterium gregoryi Argonaute is provided in SEQ ID NO: 219.

The disclosed base editors may comprise a napDNAbp domain having at least 80%, at least 85%, at least 90%, at least 95%, or at least 99% sequence identity with wild type Natronobacterium gregoryi Argonaute (SEQ ID NO: 219), which has the following amino acid sequence:

(SEQ ID NO: 219)
MTVIDLDSTTTADELTSGHTYDISVTLTGVYDNTD
EQHPRMSLAFEQDNGERRYITLWKNTTPKDVFTYD
YATGSTYIFTNIDYEVKDGYENLTATYQTTVENAT
AQEVGTTDEDETFAGGEPLDHHLDDALNETPDDAE
TESDSGHVMTSFASRDQLPEWTLHTYTLTATDGAK
TDTEYARRTLAYTVRQELYTDHDAAPVATDGLMLL
TPEPLGETPLDLDCGVRVEADETRTLDYTTAKDRL
LARELVEEGLKRSLWDDYLVRGIDEVLSKEPVLTC
DEFDLHERYDLSVEVGHSGRAYLHINFRHRFVPKL
TLADIDDDNIYPGLRVKTTYRPRRGHIVWGLRDEC
ATDSLNTLGNQSVVAYHRNNQTPINTDLLDAIEAA
DRRVVETRRQGHGDDAVSFPQELLAVEPNTHQIKQ
FASDGFHQQARSKTRLSASRCSEKAQAFAERLDPV
RLNGSTVEFSSEFFTGNNEQQLRLLYENGESVLTF
RDGARGAHPDETFSKGIVNPPESFEVAVVLPEQQA
DTCKAQWDTMADLLNQAGAPPTRSETVQYDAFSSP
ESISLNVAGAIDPSEVDAAFVVLPPDQEGFADLAS
PTETYDELKKALANMGIYSQMAYFDRFRDAKIFYT
RNVALGLLAAAGGVAFTTEHAMPGDADMFIGIDVS
RSYPEDGASGQINIAATATAVYKDGTILGHSSTRP
QLGEKLQSTDVRDIMKNAILGYQQVTGESPTHIVI
HRDGFMNEDLDPATEFLNEQGVEYDIVEIRKQPQT
RLLAVSDVQYDTPVKSIAAINQNEPRATVATFGAP
EYLATRDGGGLPRPIQIERVAGETDIETLTRQVYL
LSQSHIQVHNSTARLPITTAYADQASTHATKGYLV
QTGAFESNVGFL

### Cas9 Circular Permutants

In various embodiments, the base editors disclosed herein may comprise a circular permutant of Cas9.

The term “circularly permuted Cas9” or “circular permutant” of Cas9 or “CP-Cas9”) refers to any Cas9 protein, or variant thereof, that occurs or has been modify to engineered as a circular permutant variant, which means the N-terminus and the C-terminus of a Cas9 protein (e.g., a wild type Cas9 protein) have been topically rearranged. Such circularly permuted Cas9 proteins, or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, and Huang, T. P. et al. Circularly permuted and PAM-modified Cas9 variants broaden the targeting scope of base editors. Nat. Biotechnol. 37, 626-631 (2019). each of are incorporated herein by reference. Reference is also made to International Publication No. WO 2020/041751, published Feb. 27, 2020, herein incorporated by reference. The instant disclosure contemplates any previously known CP-Cas9 or use a new CP-Cas9 so long as the resulting circularly permuted protein retains the ability to bind DNA when complexed with a guide RNA (gRNA).

Any of the Cas9 proteins described herein, including any variant, ortholog, or naturally occurring Cas9 or equivalent thereof, may be reconfigured as a circular permutant variant.

In various embodiments, the circular permutants of Cas9 may have the following structure: N-terminus-[original C-terminus]-[optional linker]-[original N-terminus]-C-terminus.

As an example, the present disclosure contemplates the following circular permutants of canonical S. pyogenes Cas9 (1368 amino acids of UniProtKB-Q99ZW2 (CAS9_STRP1) (numbering is based on the amino acid position in SEQ ID NO: 213)): N-terminus-[1268-1368]-[optional linker]-[1-1267]-C-terminus; N-terminus-[1168-1368]-[optional linker]-[1-1167]-C-terminus; N-terminus-[1068-1368]-[optional linker]-[1-1067]-C-terminus; N-terminus-[968-1368]-[optional linker]-[1-967]-C-terminus; N-terminus-[868-1368]-[optional linker]-[1-867]-C-terminus; N-terminus-[768-1368]-[optional linker]-[1-767]-C-terminus; N-terminus-[668-1368]-[optional linker]-[1-667]-C-terminus; N-terminus-[568-1368]-[optional linker]-[1-567]-C-terminus; N-terminus-[468-1368]-[optional linker]-[1-467]-C-terminus; N-terminus-[368-1368]-[optional linker]-[1-367]-C-terminus; N-terminus-[268-1368]-[optional linker]-[1-267]-C-terminus; N-terminus-[168-1368]-[optional linker]-[1-167]-C-terminus; N-terminus-[68-1368]-[optional linker]-[1-67]-C-terminus; or N-terminus-[10-1368]-[optional linker]-[1-9]-C-terminus; or the corresponding circular permutants of other Cas9 proteins (including other Cas9 orthologs, variants, etc).

In particular embodiments, the circular permuant Cas9 has the following structure (based on S. pyogenes Cas9 (1368 amino acids of UniProtKB-Q99ZW2 (CAS9_STRP1) (numbering is based on the amino acid position in SEQ ID NO: 213): N-terminus-[102-1368]-[optional linker]-[1-101]-C-terminus; N-terminus-[1028-1368]-[optional linker]-[1-1027]-C-terminus; N-terminus-[1041-1368]-[optional linker]-[1-1043]-C-terminus; N-terminus-[1249-1368]-[optional linker]-[1-1248]-C-terminus; or N-terminus-[1300-1368]-[optional linker]-[1-1299]-C-terminus; or the corresponding circular permutants of other Cas9 proteins (including other Cas9 orthologs, variants, etc).

In still other embodiments, the circular permuant Cas9 has the following structure (based on S. pyogenes Cas9 (1368 amino acids of UniProtKB-Q99ZW2 (CAS9_STRP1) (numbering is based on the amino acid position in SEQ ID NO: 213): N-terminus-[103-1368]-[optional linker]-[1-102]-C-terminus; N-terminus-[1029-1368]-[optional linker]-[1-1028]-C-terminus; N-terminus-[1042-1368]-[optional linker]-[1-1041]-C-terminus; N-terminus-[1250-1368]-[optional linker]-[1-1249]-C-terminus; or N-terminus-[1301-1368]-[optional linker]-[1-1300]-C-terminus; or the corresponding circular permutants of other Cas9 proteins (including other Cas9 orthologs, variants, etc.).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment may correspond to the C-terminal 95% or more of the amino acids of a Cas9 (e.g., amino acids about 1300-1368), or the C-terminal 90%, 85%, 80%, 75%, 70%, 65%, 60%, 55%, 50%, 45%, 40%, 35%, 30%, 25%, 20%, 15%, 10%, 5%, or less of a Cas9. The N-terminal portion may correspond to the N-terminal 95% or more of the amino acids of a Cas9 (e.g., amino acids about 1-1300), or the N-terminal 90%, 85%, 80%, 75%, 70%, 65%, 60%, 55%, 50%, 45%, 40%, 35%, 30%, 25%, 20%, 15%, 10%, 5%, or less of a Cas9 (e.g., of SEQ ID NO: 213).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus includes or corresponds to the C-terminal 30% or less of the amino acids of a Cas9 (e.g., amino acids 1012-1368 of SEQ ID NO: 213). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., the Cas9 of SEQ ID NO: 213). In some embodiments, the C-terminal fragment that is rearranged to the N-terminus includes or corresponds to the C-terminal 410 residues or less of a Cas9 (e.g., the Cas9 of SEQ ID NO: 213). In some embodiments, the C-terminal portion that is rearranged to the N-terminus includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., the Cas9 of SEQ ID NO: 213). In some embodiments, the C-terminal portion that is rearranged to the N-terminus includes or corresponds to the C-terminal 357, 341, 328, 120, or 69 residues of a Cas9 (e.g., the Cas9 of SEQ ID NO: 213).

In other embodiments, circular permutant Cas9 variants may be defined as a topological rearrangement of a Cas9 primary structure based on the following method, which is based on S. pyogenes Cas9 of SEQ ID NO: 213: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to preceed the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (relative the S. pyogenes Cas9 of SEQ ID NO: 213) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9-CP³¹⁰, Cas9-CP¹⁰¹⁰, Cas9-CP¹⁰¹⁶, Cas9-CP¹⁰²³, Cas9-CP¹⁰²⁹, Cas9-CP¹⁰⁴¹, Cas9-CP¹²⁴⁷, Cas9-CP¹²⁴⁹, and Cas9-CP¹²⁸², respectively. This description is not meant to be limited to making CP variants from SEQ ID NO: 213, but may be implemented to make CP variants of any Cas9 sequence, either at CP sites that correspond to these positions, or at other CP sites entirely. This description is not meant to limit the specific CP sites in any way. Virtually any CP site may be used to form a CP-Cas9 variant.

Exemplary CP-Cas9 amino acid sequences, based on the Cas9 of SEQ ID NO: 213, are provided below in which linker sequences are indicated by underlining and optional methionine (M) residues are indicated in bold. It should be appreciated that the disclosure provides CP-Cas9 sequences that do not include a linker sequence, or that include different linker sequences. It should be appreciated that CP-Cas9 sequences may be based on Cas9 sequences other than that of SEQ ID NO: 213 and any examples provided herein are not meant to be limiting. Exemplary CP-Cas9 sequences are as follows:

| CP name | Sequence | SEQ ID NO: |
|---|---|---|
| CP1012 | DYKVYDVRKMIAKSEQEIGKATAKYFFYSNIMNFFKTEITLANGEIRKRPLIETN | SEQ ID NO: |
| | GETGEIVWDKGRDFATVRKVLSMPQVNIVKKTEVQTGGFSKESILPKRNSDKLIA | 225 |
| | RKKDWDPKKYGGFDSPTVAYSVLVVAKVEKGKSKKLKSVKELLGITIMERSSEEK | |
| | NPIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKE | |
| | VLDATLIHQSITGLYETRIDLSQLGGDGGSGGSGGSGGSGGSGGSGGDKKYSIGL | |
| | AIGTNSVGWAVITDEYKVPSKKFKVLGNTDRHSIKKNLIGALLFDSGETAEATRL | |
| | KRTARRRYTRRKNRICYLQEIFSNEMAKVDDSFFHRLEESFLVEEDKKHERHPIF | |
| | GNIVDEVAYHEKYPTIYHLRKKLVDSTDKADLRLIYLALAHMIKFRGHFLIEGDL | |
| | NPDNSDVDKLFIQLVQTYNQLFEENPINASGVDAKAILSARLSKSRRLENLIAQL | |
| | PGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALV | |
| | RQQLPEKYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNFDKNLPN | |
| | EKVLPKHSLLYEYFTVYNELTKVKYVTEGMRKPAFLSGEQKKAIVDLLFKTNRKV | |
| | TVKQLKEDYFKKIECFDSVEISGVEDRFNASLGTYHDLLKIIKDKDFLDNEENED | |
| | ILEDIVLTLTLFEDREMIEERLKTYAHLFDDKVMKQLKRRRYTGWGRLSRKLING | |
| | IRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSRE | |
| | RMKRIEEGIKELGSQILKEHPVENTQLQNEKLYLYYLQNGRDMYVDQELDINRLS | |
| | DYDVDHIVPQSFLKDDSIDNKVLTRSDKNRGKSDNVPSEEVVKKMKNYWRQLLNA | |
| | KLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIK | |
| | KYPKLESEFVYG | |
| CP1028 | EIGKATAKYFFYSNIMNFFKTEITLANGEIRKRPLIETNGETGEIVWDKGRDFAT | SEQ ID NO: |
| | VRKVLSMPQVNIVKKTEVQTGGFSKESILPKRNSDKLIARKKDWDPKKYGGFDSP | 226 |
| | TVAYSVLVVAKVEKGKSKKLKSVKELLGITIMERSSFEKNPIDFLEAKGYKEVKK | |
| | DLIIKLPKYSLFELENGRKRMLASAGELQKGNELALPSKYVNFLYLASHYEKLKG | |
| | SPEDNEQKQLFVEQHKHYLDEIIEQISEFSKRVILADANLDKVLSAYNKHRDKPI | |
| | REQAENIIHLFTLTNLGAPAAFKYFDTTIDRKRYTSTKEVLDATLIHQSITGLYE | |
| | TRIDLSQLGGDGGSGGSGGSGGSGGSGGSGGMDKKYSIGLAIGTNSVGWAVITDE | |
| | YKVPSKKFKVLGNTDRHSIKKNLIGALLFDSGETAEATRLKRTARRRYTRRKNRI | |
| | CYLQEIFSNEMAKVDDSFFHRLEESFLVEEDKKHERHPIFGNIVDEVAYHEKYPT | |
| | IYHLRKKLVDSTDKADLRLIYLALAHMIKFRGHFLIEGDLNPDNSDVDKLFIQLV | |
| | QTYNQLFEENPINASGVDAKAILSARLSKSRRLENLIAQLPGEKKNGLFGNLIAL | |
| | SLGLTPNFKSNFDLAEDAKLQLSKDTYDDDLDNLLAQIGDQYADLFLAAKNLSDA | |
| | ILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGS | |
| | IPHQIHLGELHAILRRQEDFYPFLKDNREKIEKILTFRIPYYVGPLARGNSRFAW | |
| | MTRKSEETITPWNFEEVVDKGASAQSFIERMTNFDKNLPNEKVLPKHSLLYEYFT | |
| | VYNELTKVKYVTEGMRKPAFLSGEQKKAIVDLLFKTNRKVTVKQLKEDYFKKIEC | |
| | FDSVEISGVEDRFNASLGTYHDLLKIIKDKDFLDNEENEDILEDIVLTLTLFEDR | |
| | EMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKELGSQ | |
| | ILKEHPVENTQLQNEKLYLYYLQNGRDMYVDQELDINRLSDYDVDHIVPQSFLKD | |
| | DSIDNKVLTRSDKNRGKSDNVPSEEVVKKMKNYWRQLLNAKLITQRKFDNLTKAE | |
| | RGGLSELDKAGFIKRQLVETRQITKHVAQILDSRMNTKYDENDKLIREVKVITLK | |
| | SKLVSDFRKDFQFYKVREINNYHHAHDAYLNAVVGTALIKKYPKLESEFVYGDYK | |
| | VYDVRKMIAKSEQ | |
| CP1041 | NIMNFFKTEITLANGEIRKRPLIETNGETGEIVWDKGRDFATVRKVLSMPQVNIV | SEQ ID NO: |
| | KKTEVQTGGFSKESILPKRNSDKLIARKKDWDPKKYGGFDSPTVAYSVLVVAKVE | 227 |
| | KGKSKKLKSVKELLGITIMERSSFEKNPIDFLEAKGYKEVKKDLIIKLPKYSLFE | |
| | LENGRKRMLASAGELQKGNELALPSKYVNFLYLASHYEKLKGSPEDNEQKQLFVE | |
| | QHKHYLDEIIEQISEFSKRVILADANLDKVLSAYNKHRDKPIREQAENIIHLFTL | |
| | TNLGAPAAFKYFDTTIDRKRYTSTKEVLDATLIHQSITGLYETRIDLSQLGGDGG | |
| | SGGSGGSGGSGGSGGSGGDKKYSIGLAIGTNSVGWAVITDEYKVPSKKFKVLGNT | |
| | DRHSIKKNLIGALLFDSGETAEATRLKRTARRRYTRRKNRICYLQEIFSNEMAKV | |
| | DDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINA | |
| | SGVDAKAILSARLSKSRRLENLIAQLPGEKKNGLFGNLIALSLGLTPNFKSNFDL | |
| | AEDAKLQLSKDTYDDDLDNLLAQIGDQYADLFLAAKNLSDAILLSDILRVNTEIT | |
| | KAPLSASMIKRYDEHHQDLTLLKALVRQQLPEKYKEIFFDQSKNGYAGYIDGGAS | |
| | QEEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPWNF | |
| | EEVVDKGASAQSFIERMTNFDKNLPNEKVLPKHSLLYEYFTVYNELTKVKYVTEG | |
| | MRKPAFLSGEQKKAIVDLLFKTNRKVTVKQLKEDYFKKIECFDSVEISGVEDRFN | |
| | ASLGTYHDLLKIIKDKDFLDNEENEDILEDIVLTLTLFEDREMIEERLKTYAHLF | |
| | DDKVMKQLKRRRYTGWGRLSRKLINGIRDKQSGKTILDFLKSDGFANRNFMQLIH | |
| | DDSLTFKEDIQKAQVSGQGDSLHEHIANLAGSPAIKKGILQTVKVVDELVKVMGR | |
| | HKPENIVIEMARENQTTQKGQKNSRERMKRIEEGIKELGSQILKEHPVENTQLQN | |
| | EKLYLYYLQNGRDMYVDQELDINRLSDYDVDHIVPQSFLKDDSIDNKVLTRSDKN | |
| | RGKSDNVPSEEVVKKMKNYWRQLLNAKLITQRKFDNLTKAERGGLSELDKAGFIK | |
| | RQLVETRQITKHVAQILDSRMNTKYDENDKLIREVKVITLKSKLVSDFRKDFQFY | |
| | KVREINNYHHAHDAYLNAVVGTALIKKYPKLESEFVYGDYKVYDVRKMIAKSEQE | |
| | IGKATAKYFFYS | |
| CP1249 | PEDNEQKQLFVEQHKHYLDEIIEQISEFSKRVILADANLDKVLSAYNKHRDKPIR | SEQ ID NO: |
| | EQAENIIHLFTLTNLGAPAAFKYFDTTIDRKRYTSTKEVLDATLIHQSITGLYET | 228 |
| | RIDLSQLGGDGGSGGSGGSGGSGGSGGSGGMDKKYSIGLAIGTNSVGWAVITDEY | |
| | KVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTI | |
| | YHLRKKLVDSTDKADLRLIYLALAHMIKFRGHFLIEGDLNPDNSDVDKLFIQLVQ | |
| | TYNQLFEENPINASGVDAKAILSARLSKSRRLENLIAQLPGEKKNGLFGNLIALS | |
| | LGLTPNFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEIFFDQS | |
| | KNGYAGYIDGGASQEEFYKFIKPILEKMDGTEELLVKLNREDLLRKQRTFDNGSI | |
| | PHQIHLGELHAILRRQEDFYPFLKDNREKIEKILTFRIPYYVGPLARGNSRFAWM | |
| | TRKSEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKIECF | |
| | DSVEISGVEDRFNASLGTYHDLLKIIKDKDFLDNEENEDILEDIVLTLTLFEDRE | |
| | MIEERLKTYAHLFDDKVMKQLKRRRYTGWGRLSRKLINGIRDKQSGKTILDFLKS | |
| | DGFANRNFMQLIHDDSLTFKEDIQKAQVSGQGDSLHEHIANLAGSPAIKKGILQT | |
| | VKVVDELVKVMGRHKPENIVIEMARENQTTQKGQKNSRERMKRIEEGIKELGSQI | |
| | LKEHPVENTQLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRKFDNLTKAER | |
| | GGLSELDKAGFIKRQLVETRQITKHVAQILDSRMNTKYDENDKLIREVKVITLKS | |
| | KLVSDFRKDFQFYKVREINNYHHAHDAYLNAVVGTALIKKYPKLESEFVYGDYKV | |
| | YDVRKMIAKSEQEIGKATAKYFFYSNIMNFFKTEITLANGEIRKRPLIETNGETG | |
| | EIVWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNF | |
| | LYLASHYEKLKGS | |
| CP1300 | KPIREQAENIIHLFTLTNLGAPAAFKYFDTTIDRKRYTSTKEVLDATLIHQSITG | SEQ ID NO: |
| | LYETRIDLSQLGGDGGSGGSGGSGGSGGSGGSGGDKKYSIGLAIGTNSVGWAVIT | 229 |
| | DEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKY | |
| | PTIYHLRKKLVDSTDKADLRLIYLALAHMIKFRGHFLIEGDLNPDNSDVDKLFIQ | |
| | LVQTYNQLFEENPINASGVDAKAILSARLSKSRRLENLIAQLPGEKKNGLFGNLI | |
| | ALSLGLTPNFKSNFDLAEDAKLQLSKDTYDDDLDNLLAQIGDQYADLFLAAKNLS | |
| | DAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDN | |
| | GSIPHQIHLGELHAILRRQEDFYPFLKDNREKIEKILTFRIPYYVGPLARGNSRF | |
| | AWMTRKSEETITPWNFEEVVDKGASAQSFIERMTNFDKNLPNEKVLPKHSLLYEY | |
| | FTVYNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGI | |
| | LQTVKVVDELVKVMGRHKPENIVIEMARENQTTQKGQKNSRERMKRIEEGIKELG | |
| | SQILKEHPVENTQLQNEKLYLYYLQNGRDMYVDQELDINRLSDYDVDHIVPQSFL | |
| | KDDSIDNKVLTRSDKNRGKSDNVPSEEVVKKMKNYWRQLLNAKLITQRKFDNLTK | |
| | AERGGLSELDKAGFIKRQLVETRQITKHVAQILDSRMNTKYDENDKLIREVKVIT | |
| | LKSKLVSDFRKDFQFYKVREINNYHHAHDAYLNAVVGTALIKKYPKLESEFVYGD | |
| | YKVYDVRKMIAKSEQEIGKATAKYFFYSNIMNFFKTEITLANGEIRKRPLIETNG | |
| | ETGEIVWDKGRDFATVRKVLSMPQVNIVKKTEVQTGGFSKESILPKRNSDKLIAR | |
| | KKDWDPKKYGGFDSPTVAYSVLVVAKVEKGKSKKLKSVKELLGITIMERSSFEKN | |
| | PIDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADANL | |
| | DKVLSAYNKHRD | |

The Cas9 circular permutants that may be useful in the base editor constructs described herein. Exemplary C-terminal fragments of Cas9, based on the Cas9 of SEQ ID NO: 213, which may be rearranged to an N-terminus of Cas9, are provided below. It should be appreciated that such C-terminal fragments of Cas9 are exemplary and are not meant to be limiting. These exemplary CP-Cas9 fragments have the following sequences:

| CP name | Sequence | SEQ ID NO: |
|---|---|---|
| CP1012 c- | DYKVYDVRKMIAKSEQEIGKATAKYFFYSNIMNFFKTEITLANGEIRKRPLIETN | SEQ ID NO: 57 |
| terminal | GETGEIVWDKGRDFATVRKVLSMPQVNIVKKTEVQTGGFSKESILPKRNSDKLIA | |
| fragment | RKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKE | |
| | VLDATLIHQSITGLYETRIDLSQLGGD | |
| CP1028 c- | EIGKATAKYFFYSNIMNFFKTEITLANGEIRKRPLIETNGETGEIVWDKGRDFAT | SEQ ID NO: 70 |
| terminal | VRKVLSMPQVNIVKKTEVQTGGFSKESILPKRNSDKLIARKKDWDPKKYGGFDSP | |
| fragment | TVAYSVLVVAKVEKGKSKKLKSVKELLGITIMERSSFEKNPIDFLEAKGYKEVKK | |
| | DLIIKLPKYSLFELENGRKRMLASAGELQKGNELALPSKYVNFLYLASHYEKLKG | |
| | SPEDNEQKQLFVEQHKHYLDEIIEQISEFSKRVILADANLDKVLSAYNKHRDKPI | |
| | REQAENIIHLFTLTNLGAPAAFKYFDTTIDRKRYTSTKEVLDATLIHQSITGLYE | |
| | TRIDLSQLGGD | |
| CP1041 c- | NIMNFFKTEITLANGEIRKRPLIETNGETGEIVWDKGRDFATVRKVLSMPQVNIV | SEQ ID NO: 77 |
| terminal | KKTEVQTGGFSKESILPKRNSDKLIARKKDWDPKKYGGFDSPTVAYSVLVVAKVE | |
| fragment | KGKSKKLKSVKELLGITIMERSSFEKNPIDFLEAKGYKEVKKDLIIKLPKYSLFE | |
| | LENGRKRMLASAGELQKGNELALPSKYVNFLYLASHYEKLKGSPEDNEQKQLFVE | |
| | QHKHYLDEIIEQISEFSKRVILADANLDKVLSAYNKHRDKPIREQAENIIHLFTL | |
| | TNLGAPAAFKYFDTTIDRKRYTSTKEVLDATLIHQSITGLYETRIDLSQLGGD | |
| CP1249 c- | PEDNEQKQLFVEQHKHYLDEIIEQISEFSKRVILADANLDKVLSAYNKHRDKPIR | SEQ ID NO: 78 |
| terminal | EQAENIIHLFTLTNLGAPAAFKYFDTTIDRKRYTSTKEVLDATLIHQSITGLYET | |
| fragment | RIDLSQLGGD | |
| CP1300 c- | KPIREQAENIIHLFTLTNLGAPAAFKYFDTTIDRKRYTSTKEVLDATLIHQSITG | SEQ ID NO: 79 |
| terminal | LYETRIDLSQLGGD | |
| fragment | | |

Cas9 Variants with Modified PAM Specificities

The base editors of the present disclosure may also comprise Cas9 variants with modified PAM specificities. In some embodiments, the Cas9 variants have expanded, or broadened, PAM specificities.

In particular embodiments, the disclosed base editors comprise a S. pyogenes Cas9-NG variant that recognizes an expanded PAM, i.e., most NG PAM sites. This variant was first reported in Nishimasu et al., Science 361, 1259-1262 (2018), incorporated herein by reference. Accordingly, in some embodiments, the base editors comprise a napDNAbp domain that comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to the SpCas9-NG set forth in SEQ ID NO: 235 below.

(SEQ ID NO: 235)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESIRPKRNSDKLIARKKDWDPKKYGGFVSPTVAYSVLVVAKVE
KGKSKKLKSVKELLGITIMERSSFEKNPIDFLEAKGYKEVKKDLIIKLPK
YSLFELENGRKRMLASARFLQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPRAFKYFDTTIDRKVYRSTKEVLDATLIHQ
SITGLYETRIDLSQLGGD

Some aspects of this disclosure provide Cas9 proteins that exhibit activity on a target sequence that does not comprise the canonical PAM (5′-NGG-3′, where N is A, C, G, or T) at its 3′-end. In some embodiments, the Cas9 protein exhibits activity on a target sequence comprising a 5′-NGG-3′ PAM sequence at its 3′-end. In some embodiments, the Cas9 protein exhibits activity on a target sequence comprising a 5′-NNG-3′ PAM sequence at its 3′-end. In some embodiments, the Cas9 protein exhibits activity on a target sequence comprising a 5′-NNA-3′ PAM sequence at its 3′-end. In some embodiments, the Cas9 protein exhibits activity on a target sequence comprising a 5′-NNC-3′ PAM sequence at its 3′-end. In some embodiments, the Cas9 protein exhibits activity on a target sequence comprising a 5′-NNT-3′ PAM sequence at its 3′-end. In some embodiments, the Cas9 protein exhibits activity on a target sequence comprising a 5′-NGT-3′ PAM sequence at its 3′-end. In some embodiments, the Cas9 protein exhibits activity on a target sequence comprising a 5′-NGA-3′ PAM sequence at its 3′-end. In some embodiments, the Cas9 protein exhibits activity on a target sequence comprising a 5′-NGC-3′ PAM sequence at its 3′-end. In some embodiments, the Cas9 protein exhibits activity on a target sequence comprising a 5′-NAA-3′ PAM sequence at its 3′-end. In some embodiments, the Cas9 protein exhibits activity on a target sequence comprising a 5′-NAC-3′ PAM sequence at its 3′-end. In some embodiments, the Cas9 protein exhibits activity on a target sequence comprising a 5′ NAT-3′ PAM sequence at its 3′-end. In still other embodiments, the Cas9 protein exhibits activity on a target sequence comprising a 5′-NAG-3′ PAM sequence at its 3′-end.

In some embodiments, the disclosed base editors comprise a napDNAbp domain comprising a SpCas9-NG, which has a PAM that corresponds to NGN. In some embodiments, the disclosed base editors comprise a napDNAbp domain comprising a SpCas9-KKH, which has a PAM that corresponds to NNNRRT.

It should be appreciated that any of the amino acid mutations described herein, (e.g., A262T) from a first amino acid residue (e.g., A) to a second amino acid residue (e.g., T) may also include mutations from the first amino acid residue to an amino acid residue that is similar to (e.g., conserved) the second amino acid residue. For example, mutation of an amino acid with a hydrophobic side chain (e.g., alanine, valine, isoleucine, leucine, methionine, phenylalanine, tyrosine, or tryptophan) may be a mutation to a second amino acid with a different hydrophobic side chain (e.g., alanine, valine, isoleucine, leucine, methionine, phenylalanine, tyrosine, or tryptophan). For example, a mutation of an alanine to a threonine (e.g., a A262T mutation) may also be a mutation from an alanine to an amino acid that is similar in size and chemical properties to a threonine, for example, serine. As another example, mutation of an amino acid with a positively charged side chain (e.g., arginine, histidine, or lysine) may be a mutation to a second amino acid with a different positively charged side chain (e.g., arginine, histidine, or lysine). As another example, mutation of an amino acid with a polar side chain (e.g., serine, threonine, asparagine, or glutamine) may be a mutation to a second amino acid with a different polar side chain (e.g., serine, threonine, asparagine, or glutamine). Additional similar amino acid pairs include, but are not limited to, the following: phenylalanine and tyrosine; asparagine and glutamine; methionine and cysteine; aspartic acid and glutamic acid; and arginine and lysine. The skilled artisan would recognize that such conservative amino acid substitutions will likely have minor effects on protein structure and are likely to be well tolerated without compromising function. In some embodiments, any amino of the amino acid mutations provided herein from one amino acid to a threonine may be an amino acid mutation to a serine. In some embodiments, any amino of the amino acid mutations provided herein from one amino acid to an arginine may be an amino acid mutation to a lysine. In some embodiments, any amino of the amino acid mutations provided herein from one amino acid to an isoleucine, may be an amino acid mutation to an alanine, valine, methionine, or leucine. In some embodiments, any amino of the amino acid mutations provided herein from one amino acid to a lysine may be an amino acid mutation to an arginine. In some embodiments, any amino of the amino acid mutations provided herein from one amino acid to an aspartic acid may be an amino acid mutation to a glutamic acid or asparagine. In some embodiments, any amino of the amino acid mutations provided herein from one amino acid to a valine may be an amino acid mutation to an alanine, isoleucine, methionine, or leucine. In some embodiments, any amino of the amino acid mutations provided herein from one amino acid to a glycine may be an amino acid mutation to an alanine. It should be appreciated, however, that additional conserved amino acid residues would be recognized by the skilled artisan and any of the amino acid mutations to other conserved amino acid residues are also within the scope of this disclosure.

In some embodiments, the present disclosure may utilize any of the Cas9 variants disclosed in the SEQUENCES section below.

In some embodiments, the Cas9 protein comprises a combination of mutations that exhibit activity on a target sequence comprising a 5′-NAA-3′ PAM sequence at its 3′-end. In some embodiments, the combination of mutations is present in any one of the clones listed in Table 1. In some embodiments, the combination of mutations is conservative mutations of the clones listed in Table 1. In some embodiments, the Cas9 protein comprises the combination of mutations of any one of the Cas9 clones listed in Table 1.

TABLE 1
NAA PAM Clones
Mutations from wild-type SpCas9 (e.g., SEQ ID NO: 213)

D177N, K218R, D614N, D1135N, P1137S, E1219V, A1320V, A1323D, R1333K
D177N, K218R, D614N, D1135N, E1219V, Q1221H, H1264Y, A1320V, R1333K
A10T, 1322V, S409I, E427G, G715C, D1135N, E1219V, Q1221H, H1264Y, A1320V, R1333K
A367T, K710E, R1114G, D1135N, P1137S, E1219V, Q1221H, H1264Y, A1320V, R1333K
A10T, 1322V, S409I, E427G, R753G, D861N, D1135N, K1188R, E1219V, Q1221H, H1264H,
A1320V, R1333K
A10T, 1322V, S409I, E427G, R654L, V743I, R753G, M1021T, D1135N, D1180G, K1211R,
E1219V, Q1221H, H1264Y, A1320V, R1333K
A10T, 1322V, S409I, E427G, V743I, R753G, E762G, D1135N, D1180G, K1211R, E1219V,
Q1221H, H1264Y, A1320V, R1333K
A10T, 1322V, S409I, E427G, R753G, D1135N, D1180G, K1211R, E1219V, Q1221H, H1264Y,
S1274R, A1320V, R1333K
A10T, 1322V, S409I, E427G, A589S, R753G, D1135N, E1219V, Q1221H
R1333K TL, 60 TL, 65
A10T, 1322V, S409I, E427G, R753G, E757K, G865G, D1135N, E1219V, Q1221H, H1264Y,
A1320V, R1333K
A10T, 1322V, S409I, E427G, R654L, R753G, E757K, D1135N, E1219V, Q1221H, H1264Y,
A1320V, R1333K
A10T, 1322V, S409I, E427G, K599R, M631A, R654L, K673E, V743I, R753G, N758H,
D1135N, D1180G, E1219V, Q1221H, Q1256R, H1264Y, A1320V, A1323D, R1333K TL, 60
A10T, 1322V, S409I, E427G, R654L, K673E, V743I, R753G, E762G, N869S, N1054D R1114G,
D1135N, D1180G, E1219V, Q1221H, H1264Y, A1320V, A1323D, R1333K
A10T, 1322V, S409I, E427G, R654L, L727I, V743I, R753G, E762G, R859S, N946D, F1134L,
D1135N, D1180G, E1219V, Q1221H, H1264Y, N1317T, A1320V, A1323D, R1333K
A10T, 1322V, S409I, E427G, R654L, K673E, V743I, R753G, E762G, N803S, N869S, Y1016D,
G1077D, R1114G, F1134L, D1 135N, D1180G, E1219V, Q1221H, H1264Y, V1290G, L1318S,
A1320V, A1323D, R1333K
A10T, 1322V, S409I, E427G, R654L, K673E, V743I, R753G, E762G, N803S, N869S, Y1016D,
G1077D, R1114G, F1134L, D1 135N, K1151E, D1180G, E1219V, Q1221H, H1264Y, V1290G,
L1318S, A1320V, R1333K
A10T, 1322V, S409I, E427G, R654L, K673E, V743I, R753G, E762G, N803S, N869S, Y1016D,
G1077D, R1114G, F1134L, D1 135N, D1180G, E1219V, Q1221H, H1264Y, V1290G, L1318S,
A1320V, A1323D, R1333K
A10T, 1322V, S409I, E427G, R654L, K673E, F693L, V743I, R753G, E762G, N803S, N869S,
L921P, Y1016D, G1077D, F1080S, R1114G, D1135N, D1180G, E1219V, Q1221H, H1264Y,
L1318S, A1320V, A1323D, R1333K
A10T, 1322V, S409I, E427G, E630K, R654L, K673E, V743I, R753G, E762G, Q768H, N803S,
N869S, Y1016D, G1077D, R1114G, F1134L, D1135N, D1180G, E1219V, Q1221H, H1264Y,
L1318S, A1320V, R1333K
A10T, 1322V, S409I, E427G, R654L, K673E, F693L, V743I, R753G, E762G, Q768H, N803S,
N869S, Y1016D, G1077D, R1114G, F1134L, D1135N, D1180G, E1219V, Q1221H, G1223S,
H1264Y, L1318S, A1320V, R1333K
A10T, 1322V, S409I, E427G, R654L, K673E, F693L, V743I, R753G, E762G, N803S, N869S,
L921P, Y1016D, G1077D, F1801S, R1114G, D1135N, D1180G, E1219V, Q1221H, H1264Y,
L1318S, A1320V, A1323D, R1333K
A10T, 1322V, S409I, E427G, R654L, V743I, R753G, M1021T D1135N, D1180G, K1211R,
E1219V, Q1221H, H1264Y, A1320V, R1333K
A10T, 1322V, S409I, E427G, R654L, K673E, V743I, R753G, E762G, M673I, N803S, N869S,
G1077D, R1114G, D1135N, V1139A, D1180G, E1219V, Q1221H A1320V, R1333K
A10T, 1322V, S409I, E427G, R654L, K673E, V743I, R753G, E762G, N803S, N869S, R1114G,
D1135N, E1219V, Q1221H, A1320V, R1333KTZ, 1/70

In some embodiments, the Cas9 protein comprises an amino acid sequence that is at least 80% identical to the amino acid sequence of a Cas9 protein as provided by any one of the variants of Table 1. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 1.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 213. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 213 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes as provided by SEQ ID NO: 213 on the same target sequence. In some embodiments, the 3′ end of the target sequence is directly adjacent to an AAA, GAA, CAA, or TAA sequence. In some embodiments, the Cas9 protein comprises a combination of mutations that exhibit activity on a target sequence comprising a 5′-NAC-3′ PAM sequence at its 3′-end. In some embodiments, the combination of mutations is present in any one of the clones listed in Table 2. In some embodiments, the combination of mutations is conservative mutations of the clones listed in Table 2. In some embodiments, the Cas9 protein comprises the combination of mutations of any one of the Cas9 clones listed in Table 2.

TABLE 2
NAC PAM Clones
MUTATIONS FROM WILD-TYPE SPCAS9 (E.G., SEQ ID NO: 213)

T472I, R753G, K890E, D1332N, R1335Q, T1337N
I1057S, D1135N, P1301S, R1335Q, T1337N
T472I, R753G, D1332N, R1335Q, T1337N
D1135N, E1219V, D1332N, R1335Q, T1337N
T472I, R753G, K890E, D1332N, R1335Q, T1337N
I1057S, D1135N, P1301S, R1335Q, T1337N
T472I, R753G, D1332N, R1335Q, T1337N
T472I, R753G, Q771H, D1332N, R1335Q, T1337N
E627K, T638P, K652T, R753G, N803S, K959N, R1114G, D1135N, E1219V, D1332N, R1335Q,
T1337N
E627K, T638P, K652T, R753G, N803S, K959N, R1114G, D1135N, K1156E, E1219V, D1332N,
R1335Q, T1337N
E627K, T638P, V647I, R753G, N803S, K959N, G1030R, I1055E, R1114G, D1135N, E1219V,
D1332N, R1335Q, T1337N
E627K, E630G, T638P, V647A, G687R, N767D, N803S, K959N,
D1332G, R1335Q, T1337N
E627K, T638P, R753G, N803S, K959N, R1114G D1135N, E1219V, N1266H, D1332N, R1335Q,
T1337N
E627K, T638P, R753G, N803S, K959N, I1057T R1114G, D1135N, E1219V, D1332N, R1335Q,
T1337N
E627K, T638P, R753G, N803S, K959N, R1114G D1135N, E1219V, D1332N, R1335Q, T1337N
E627K, M631I, T638P, R753G, N803S, K959N, Y1036H, R1114G, D1135N, E1219V, D1251G,
D1332G, R1335Q, T1337N
E627K, T638P, R753G, N803S, V875I, K959N, Y1016C, R1114G, D1135N, E1219V, D1251G,
D1332G, R1335Q, T1337N, 11348V
K608R, E627K, T638P, V647I, R654L, R753G, N803S, T804A,
D1135N, E1219V, D1332N, R1335Q, TI 337N
K608R, E627K, T638P, V647I, R753G, N803S, V922A, K959N,
D1135N, K1156N, E1219V, N1252D, D1332N, R1335Q, T1337N
K608R, E627K, R629G, T638P, V647I, A711T, R753G, K775R, K789E, N803S, K959N, V1015A,
Y1036H, R1114G, D1135N, E1219V, N1286H, D1332N, R1335Q, T1337N
K608R, E627K, T638P, V647I, T740A, R753G, N803S, K948E, K959N, Y1016S, R1114G,
D1135N, E1219V, N1286H, D1332N, R1335Q, T1337N
K608R, E627K, T638P, V647I, T740A, N803S, K948E, K959N,
E1219V, N1286H, D1332N, R1335Q, T1337N
I670S, K608R, E627K, E630G, T638P, V647I, R653K, R753G, I795L, K797N, N803S, K866R,
K890N, K959N, Y1016C, R1114G, D1135N, E1219V, D1 332N, R1335Q, T1337N
K608R, E627K, T638P, V647I, T740A, G752R, R753G, K797N,
Y1016S, R1114G, D1135N, E1219V, N1266H, D1332N, R1335Q, T1337N
I570T, A589V, K608R, E627K, T638P, V647I, R654L, Q716R, R753G, N803S, K948E, K959N,
Y1016S, R1114G, D1135N, E1207G, E1219V, N1234D, D1332N, R1335Q T1337N
K608R, E627K, R629G, T638P, V647I, R654L, Q740R, R753G, N803S, K959N, N990S, T995S,
V1015A, Y1036D, R1114G, D1135N, E1207G, E1219V, N1234D, N1266H D1332N, R1335Q,
T1337N
I562F, V565D, I570T, K608R, L625S, E627K, T638P, V647I, R654I, G752R, R753G,
N808D, K959N, M1021L, R1114G, D1135N, N1177S, N1234D, 1332N, R1335Q, T1337N
I562F, I570T, K608R, E627K, T638P, V647I, R753G, E790A, N803S, K959N, V1015A, Y1036H,
R1114G, D1135N, D1180E, A1184T, E1219V, D1332N, R1335Q, T1337N
I570T, K608R, E627K, T638P, V647I, R654H, R753G, E790A, N803S, K959N, V1015A, R1114G,
D1127A, D1135N, E1219V, D1332N, R1335Q, T1337N
I570T, K608R, L625S, E627K, T638P, V647I, R654I, T703P, R753G, N803S, N808D, K959N,
M1021L, R1114G, D1135N, E1219V, D1332N, R1335Q, T1337N
I570S, K608R, E627K, E630G, T638P, V647I, R653K, R753G, I795L, N803S, K866R, K890N,
K959N, Y1016C, R1114G, D1135N, E1219V, D1332N, R1335Q, T1337N
I570T, K608R, E627K, T638P, V647I, R654H, R753G, E790A, N803S, K959N, V1016A, R1114G,
D1135N, E1219V, K1246E, D1332N, R1335Q, T1337N
K608R, E627K, T638P, V647I, R654L, K673E, R753G, E790A, N803S, K948E, K959N, R1114G,
D1127G, D1135N, D1180E, E1219V, N1286H, D1332N, R1335Q, T1337N
K608R, L625S, E627K, T638P, V647I, R654I, I670T, R753G, N803S, N808D, K959N, M1021L,
R1114G, D1135N, E1219V, N1286H, D1332N, R1335Q, T1337N
E627K, M631V, T638P, V647I, K710E, R753G, N803S, N808D, K948E, M1021L R1114G,
D1135N, E1219V, D1332N, R1335Q, T1337N, S1338T, H1349R

In some embodiments, the Cas9 protein comprises an amino acid sequence that is at least 80% identical to the amino acid sequence of a Cas9 protein as provided by any one of the variants of Table 2. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 2.

In some embodiments, the Cas9 protein comprises a combination of mutations that exhibit activity on a target sequence comprising a 5′-NAT-3′ PAM sequence at its 3′-end. In some embodiments, the combination of mutations is present in any one of the clones listed in Table 3. In some embodiments, the combination of mutations is conservative mutations of the clones listed in Table 3. In some embodiments, the Cas9 protein comprises the combination of mutations of any one of the Cas9 clones listed in Table 3.

TABLE 3
NAT PAM Clones
MUTATIONS FROM WILD-TYPE SPCAS9 (E.G., SEQ ID NO: 213)

K961E, H985Y, D1135N, K1191N, E1219V, Q1221H, A1320A, P1321S, R1335L
D1135N, G1218S, E1219V, Q1221H, P1249S, P1321S, D1322G, R1335L
V743I, R753G, E790A, D1135N, G1218S, E1219V, Q1221H, A1227V, P1249S, N1286K, A1293T,
P1321S, D1322G, R1335L, T1339I
F575S, M631L, R654L, V748I, V743I, R753G, D853E, V922A, R1114G D1135N, G1218S,
E1219V, Q1221H, A1227V, P1249S, N1286K, A1293T, P1321S, D1322G, R1335L, T1339I
F575S, M631L, R654L, R664K, R753G, D853E, V922A, R1114G D1135N, D1180G, G1218S,
E1219V, Q1221H, P1249S, N1286K, P1321S, D1322G, R1335L
M631L, R654L, R753G, K797E, D853E, V922A, D1012A, R1114G D1135N, G1218S, E1219V,
Q1221H, P1249S, N1317K, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, D596Y, M631L, R654L, R664K, R753G, D853E, V922A, R1114G, Y1131C, D1135N,
D1180G, G1218S, E1219V, Q1221H, P1249S, Q1256R, P1321S, D1322G, R1335L
F575S, M631L, R654L, R664K, K710E, V750A, R753G, D853E, V922A, R1114G, Y1131C,
D1135N, D1180G, G1218S, E1219V, Q1221H, P1249S, P1321S, D1322G, R1335L
F575S, M631L, K649R, R654L, R664K, R753G, D853E, V922A, R1114G, Y1131C, D1135N,
K1156E, D1180G, G1218S, E1219V, Q1221H, P1249S, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, M631L, R654L, R664K, R753G, D853E, V922A, I1057G, R1114G, Y1131C, D1135N,
D1180G, G1218S, E1219V, Q1221H, P1249S, N1308D, P1321S, D1322G, R1335L
M631L, R654L, R753G, D853E, V922A, R1114G, Y1131C, D1135N, E1150V, D118 OG, G1218S,
E1219V, Q1221H, P1249S, P1321S, D1332G, R1335L
M631L, R654L, R664K, R753G, D853E, 11057V, Y1131C, D1135N, D1180G, G121 8S, E1219V,
Q1221H, P1249S, P1321S, D1332G, R1335L
M631L, R654L, R664K, R753G, 11057V, R1114G, Y1131C, D1135N, D1180G, G12 18S, E1219V,
Q1221H, P1249S, P1321S, D1332G, R1335L

The above description of various napDNAbps which can be used in connection with the presently disclose base editors is not meant to be limiting in any way. The base editors may comprise the canonical SpCas9, or any ortholog Cas9 protein, or any variant Cas9 protein-including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats). The base editors described herein may also comprise Cas9 equivalents, including Cas12a/Cpf1 and Cas12b proteins which are the result of convergent evolution. The napDNAbps used herein (e.g., SpCas9, Cas9 variant, or Cas9 equivalents) may also contain various modifications that alter/enhance their PAM specifities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a references SpCas9 canonical sequences or a reference Cas9 equivalent (e.g., Cas12a/Cpf1).

In a particular embodiment, the Cas9 variant having expanded PAM capabilities is SpCas9 (H840A) VRQR, or SpCas9-VRQR. In some embodiments, the disclosed base editors comprise a napDNAbp domain that has a sequence that is at least 90%, at least 95%, at least 98%, or at least 99% identical to SpCas9-VRQR. In some embodiments, the disclosed base editors comprise a napDNAbp domain that comprises SpCas9-VRQR. The SpCas9-VRQR comprises the following amino acid sequence (with the V, R, Q, R substitutions relative to the SpCas9 (H840A) of SEQ ID NO: 222 show, in bold underline. In addition, the methionine residue in SpCas9 (H840) was removed for SpCas9 (H840A) VRQR):

(SEQ ID NO: 223)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASARELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKQYRSTKEVLDATLIHQS
ITGLYETRIDLSQLGGD

In another particular embodiment, the Cas9 variant having expanded PAM capabilities is SpCas9 (H840A) VRER, having the following amino acid sequence (with the V, R, E, R substitutions relative to the SpCas9 (H840A) of SEQ ID NO: 222 are shown in bold underline. In addition, the methionine residue in SpCas9 (H840) was removed for SpCas9 (H840A) VRER):

(SEQ ID NO: 224)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASARELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKEYRSTKEVLDATLIHQS
ITGLYETRIDLSQLGGD

In addition, any available methods may be utilized to obtain or construct a variant or mutant Cas9 protein. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Because of their nature, gain-of-function mutations are usually dominant.

Evolved napDNAbp Domains

This disclosure provides napDNAbp domains that may comprise Cas9 variants that have been evolved by continuous or non-continuous evolution and recognize an expanded PAM, as recently reported in Hu et al., Nature, 556(7699):57-63 (2018) and International Publication No. WO 2020/041751, published Feb. 27, 2020, each of which is incorporated by reference herein. Exemplary evolved Cas9 variants having expanded PAM specificities include xCas9(3.6) and xCas9(3.7).

Phage-assisted continuous evolution (PACE) was used to evolve the wild type SpCas9 to recognize a broad range of PAM sequences, including HAA (e.g., GAA), NAA, NAG, HAT (e.g., GAT), and HAC PAM sequences. The PAM compatibility of xCas9 is the broadest reported to date among Cas9s active in mammalian cells, and supports applications in human cells including targeted transcriptional activation, nuclease-mediated gene disruption, and both cytosine and adenine base editing.

Accordingly, in some embodiments, the base editors comprise a napDNAbp domain that comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to the xCas9 set forth in one of SEQ ID NOs: 236 or 237, provided below. Residues that have been mutated to wild-type SpCas9 (SEQ ID NO: 213) are underlined and bolded.

(xCas9(3.7), SEQ ID NO: 236)
MDKKYSIGLDIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLNPDNSDVDKLFIQ
LVQTYNQLFEENPINASGVDAKAILSARLSKSRRLENLIAQLPGEKKNGLFGNLIALSLGLTP
NFKSNFDLAEDTKLQLSKDTYDDDLDNLLAQIGDQYADLFLAAKNLSDAILLSDILRVNTEI
TKAPLSASMIKLYDEHHQDLTLLKALVRQQLPEKYKEIFFDQSKNGYAGYIDGGASQEEFY
KFIKPILEKMDGTEELLVKLNREDLLRKQRTFDNGIIPHQIHLGELHAILRRQEDFYPFLKDN
REKIEKILTFRIPYYVGPLARGNSRFAWMTRKSEETITPWNFEKVVDKGASAQSFIERMTNFD
KNLPNEKVLPKHSLLYEYFTVYNELTKVKYVTEGMRKPAFLSGDQKKAIVDLLFKTNRKVT
VKQLKEDYFKKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLKS
DGFANRNFIQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKN
PIDFLEAKGYKEVKKDLIIKLPKYSLFELENGRKRMLASAGVLQKGNELALPSKYVNFLYLA
SHYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQLG
GD
(xCas9(3.6), SEQ ID NO: 237)
MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVGEDKKHERHPIFGNIVDE
VAYHEKYPTIYHLRKKLVDSTDKADLRLIYLALAHMIKFRGHFLIEGDLNPDNSDVDKLFIQ
LVQTYNQLFEENPINASGVDAKAILSARLAKSRRLENLIAQLPGEKKNGLFGNLIALSLGLTP
NFKSNFDLAEDTKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEEFYK
FIKPILEKMDGTEELLVKLNREDLLRKQRTFDNGIIPHQIHLGELHAILRRQEDFYPFLKDNRE
KIEKILTFRIPYYVGPLARGNSRFAWMTRKSEETITPWNFEKVVDKGASAQSFIERMTNFDK
NLPNEKVLPKHSLLYEYFTVYNELTKVKYVTEGMRKPAFLSGDQKKAIVDLLFKTNRKVTV
KQLKEDYFKKIECFDSVEISGVEDRFNASLGTYHDLLKIIKDKDFLDNEENEDILEDIVLTLTL
FEDREMIEERLKTYAHLFDDKVMKQLKRRRYTGWGRLSRKLINGIRDKQSGKTILDFLKSD
GFANRNFIQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQLQNEK
LYLYYLQNGRDMYVDQELDINRLSDYDVDHIVPQSFLKDDSIDNKVLTRSDKNRGKSDNVP
SEEVVKKMKNYWRQLLNAKLITQRKFDNLTKAERGGLSELDKAGFIKRQLVETRQITKHVA
QILDSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLANG
EIRKRPLIETNGETGEIVWDKGRDFATVRKVLSMPQVNIVKKTEVQTGGFSKESILPKRNSD
KLIARKKDWDPKKYGGFDSPTVAYSVLVVAKVEKGKSKKLKSVKELLGITIMERSSFEKNPI
DFLEAKGYKEVKKDLIIKLPKYSLFELENGRKRMLASAGVLQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDISQLGG
D

Exemplary evolved Cas9 variants, such as xCas9(3.6) and xCas9(3.7), may be mutated into a nuclease-inactive Cas9 variant (or dxCas9) by introducing both of the D10A and H840A substitutions as described above. Exemplary evolved Cas9 variants may be mutated into a Cas9 nickase variant (or xCas9n) by introducing either of the D10A and H840A substitutions as described above. Thus, in some embodiments, the disclosed base editors comprise a napDNAbp domain that comprises a dxCas9 or an xCas9n.

Any available methods may be utilized to obtain an evolved variant or mutant Cas9 protein. Mutations can be introduced into a reference Cas9 protein using site-directed mutagenesis. Older methods of site-directed mutagenesis known in the art rely on sub-cloning of the sequence to be mutated into a vector, such as an M13 bacteriophage vector, that allows the isolation of a single-stranded DNA template. In these methods, one anneals a mutagenic primer (i.e., a primer capable of annealing to the site to be mutated but bearing one or more mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation. More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

Mutations may also be introduced by directed evolution processes, such as phage-assisted continuous evolution (PACE) or phage-assisted noncontinuous evolution (PANCE). The term “phage-assisted continuous evolution (PACE),” as used herein, refers to continuous evolution that employs phage as viral vectors. The general concept of PACE technology has been described, for example, in International Application No. PCT/US2009/056194, filed Sep. 8, 2009, published as WO 2010/028347 on Mar. 11, 2010; International Application No. PCT/US2011/066747, filed Dec. 22, 2011, published as WO 2012/088381 on Jun. 28, 2012; U.S. Application, U.S. Pat. No. 9,023,594, issued May 5, 2015, International Application No. PCT/US2015/012022, filed Jan. 20, 2015, published as WO 2015/134121 on Sep. 11, 2015, U.S. Pat. No. 10,179,911, issued Jan. 15, 2019; International PCT Application, PCT/US2016/027795, filed Apr. 15, 2016, published as WO 2016/168631 on Oct. 20, 2016, and International Patent Publication WO 2019/023680, published Jan. 31, 2019, the entire contents of each of which are incorporated herein by reference. Variant Cas9s may also be generated by phage-assisted non-continuous evolution (PANCE), which as used herein, refers to non-continuous evolution that employs phage as viral vectors. PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

Any of the napDNAbp domains disclosed herein may be provided as an isolated napDNAbp protein, e.g., for use in the assays and systems for determining off-target effects disclosed herein. In some embodiments, isolated Cas9 proteins are provided. In some embodiments, isolated dCas9 proteins are provided. In other embodiments, isolated nCas9 proteins are provided. In other embodiments, isolated CP1028, SpCas9-NG, and/or xCas9 proteins are provided. These isolated proteins may be associated with a gRNA engineered to bind the protein.

The isolated napDNAbp proteins provided herein may be from any bacterial species. In some embodiments, the isolated napDNAbp proteins are derived from S. pyogenes and/or S. aureus.

Any of the references noted above which relate to napDNAbp domains are hereby incorporated by reference in their entireties, if not already stated so.

### Cytidine Deaminases

In various embodiments, the novel base editors provided herein comprise a nucleobase modification domain that comprises a cytidine deaminase domain. In some embodiments, the cytidine deaminase domain is capable of catalyzing a C to U base conversion through a deamination reaction. The U is ultimately converted to a T by the cell's replication and mismatch repair systems.

In some embodiments, the cytidine deaminase is an apolipoprotein B mRNA-editing complex (APOBEC) deaminase. In some embodiments, the cytidine deaminase is an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, or an APOBEC4 deaminase, or a variant thereof. In some embodiments, the deaminase is an activation-induced deaminase (AID), e.g., a human AID. In some embodiments, the deaminase is a Lamprey CDA1, e.g., a Petromyzon marinus cytidine deaminase 1 (pmCDA1).

In some embodiments, the deaminase is from a human, chimpanzee, gorilla, monkey, cow, dog, rat, or mouse. In some embodiments, the deaminase is from a human. In some embodiments the deaminase is from a rat.

In some embodiments, the deaminase is a rat APOBEC1 deaminase comprising the amino acid sequence set forth in (SEQ ID NO: 238), or a variant thereof. In some embodiments, the deaminase is a human APOBEC1 deaminase comprising the amino acid sequence set forth in (SEQ ID NO: 239), or a variant thereof. In some embodiments, the deaminase is pmCDA1 (CDA) (SEQ ID NO: 244, or a variant thereof. Variants of rat APOBEC1 and CD1 that were evolved by PACE (evoAPOBEC1 and evoCDA1, respectively) were disclosed in Thuronyi et al., Nat. Biotechnol. (September 2019), 37(9):1070-1079 and International Publication No. WO 2019/023680, each of which is herein incorporated by reference. In some embodiments, the deaminase is evoCDA (SEQ ID NO: 246) or evoAPOBEC1 (SEQ ID NO: 247).

In some embodiments, the deaminase is human APOBEC3G (A3G) (SEQ ID NO: 242), or an evolved variant thereof. In some embodiments, the deaminase is a human APOBEC3B (SEQ ID NO: 241), or an evolved variant thereof. In some embodiments, the deaminase is a human APOBEC3A, or A3A (SEQ ID NO: 240), or an evolved variant thereof. In some embodiments, the deaminase is an AID (SEQ ID NO: 243), or an evolved variant thereof. In some embodiments, the deaminase is an evolved APOBEC3A (eA3A) (SEQ ID NO: 245), such as an APOBEC3A engineered to have a strict 5′ T sequence context requirement, as provided in M. Gehrke et al., APOBEC3A (eA3A). In some embodiments, the deaminase is at least 80%, at least 85%, at least 90%, at least 92%, at least 95%, at least 96%, at least 97%, at least 98%, at least 99%, or at least 99.5% identical to any one of the amino acid sequences set forth in SEQ ID NOs: 238-247.

In still other embodiments, the deaminase is a variant of rat APOBEC1. In particular embodiments, the deaminase is selected from YE1, YEE, YE2, EE, R33A, R33A+K34A, or AALN, or a variant thereof. The deaminase may comprise any of the variants of rat APOBEC1 reported in J. Grunewald et al., Nature 569, 433-437 (2019) and J. M. Gehrke et al., Nat. Biotechnol. 36, 977-982 (2018), each of which is incorporated herein by reference. In particular embodiments, the deaminase is a YE1 (SEQ ID NO: 248). In some embodiments, the deaminase is a YEE (SEQ ID NO: 249). In some embodiments, the deaminase is a YE2 (SEQ ID NO: 250). In some embodiments, the deaminase is a EE (SEQ ID NO: 251). The YE1, YEE, YE2, and EE variants are disclosed in International Publication No. WO 2018/0176009, herein incorporated by reference.

Grunewald et al. recently reported APOBEC1 mutants with R33A and R33A+K34A mutations, which conferred lower off-target RNA editing. Thus, in some embodiments, the cytidine deaminase comprises R33A (SEQ ID NO: 252) or R33A+K34A (SEQ ID NO: 253) variants. To further increase the target sequence compatibility of R33A+K34A-BE4, which exhibits a relatively stringent 5′-TC requirement for base editing, H122L and D124N, two mutations that were recently found during the continuous evolution of APOBEC1 to enable efficient deamination of 5′-GC substrates, was engineered and disclosed in Thuronyi et al. The resulting R33A+K34A+H122L+D124N variant is hereafter referred to as AALN. Thus, in some embodiments, the deaminase is an AALN (SEQ ID NO: 254). The AALN variant is also disclosed in International Publication No. WO 2019/023680. In some embodiments, the deaminase is at least 80%, at least 85%, at least 90%, at least 92%, at least 95%, at least 96%, at least 97%, at least 98%, at least 99%, or at least 99.5% identical to any one of the amino acid sequences set forth in SEQ ID NOs: 248-254.

Additionally, FERNY, a truncated, ancestrally reconstructed deaminase, which lacks an RNA-binding motif that could mediate nonspecific interactions with nucleic acids, was reported in Thuronyi et al. An evolved FERNY (evoFERNY), comprising an H102P and D104N substitutions, that was engineered by PACE to possess improved deamination of 5′-GC substrates, was also disclosed in Thuronyi et al. Accordingly, in other embodiments, the deaminase is a FERNY (SEQ ID NO: 255) or an evoFERNY (SEQ ID NO: 256).

Some aspects of the disclosure are based on the recognition that modulating the deaminase domain catalytic activity of any of the CBEs provided herein, for example by making point mutations in the deaminase domain, affect the processivity of the CBEs. For example, mutations that reduce, but do not eliminate, the catalytic activity of a deaminase domain of the base editor can make it less likely that the deaminase domain will catalyze the deamination of a residue adjacent to a target residue, thereby narrowing the deamination window. The ability to narrow the deaminataion window may prevent unwanted deamination of residues adjacent of specific target residues, which may decrease or prevent off-target effects.

Exemplary suitable deaminase domains that can be fused to Cas9 domains according to aspects of this disclosure are provided below. It should be understood that, in some embodiments, the active domain of the respective sequence can be used, e.g., the domain without a localizing signal (nuclear localization sequence, without nuclear export signal, cytoplasmic localizing signal).

In some embodiments, the cytidine deaminase domain of the disclosed base editors is at least 95%, at least 96%, at least 97%, at least 98%, at least 99%, or at least 99.5% identical to the deaminase domain of any one of SEQ ID NOs: 238-256, provided below. In some embodiments, the cytidine deaminase domain comprises the amino acid sequence of any one of SEQ ID NOs: 238-256.

Rat APOBEC1
(SEQ ID NO: 238)
MSSETGPVAVDPTLRRRIEPHEFEVFFDPRELRKETCLLYEINWGGRHSIWRHTSQNTNKHV
EVNFIEKFTTERYFCPNTRCSITWFLSWSPCGECSRAITEFLSRYPHVTLFIYIARLYHHADPR
NRQGLRDLISSGVTIQIMTEQESGYCWRNFVNYSPSNEAHWPRYPHLWVRLYVLELYCIILG
LPPCLNILRRKQPQLTFFTIALQSCHYQRLPPHILWATGLK
Human APOBEC1
(SEQ ID NO: 239)
MTSEKGPSTGDPTLRRRIEPWEFDVFYDPRELRKEACLLYEIKWGMSRKIWRSSGKNTTNH
VEVNFIKKFTSERDFHPSMSCSITWFLSWSPCWECSQAIREFLSRHPGVTLVIYVARLFWHM
DQQNRQGLRDLVNSGVTIQIMRASEYYHCWRNFVNYPPGDEAHWPQYPPLWMMLYALEL
HCIILSLPPCLKISRRWQNHLTFFRLHLQNCHYQTIPPHILLATGLIHPSVAWR
Human APOBEC3A
(SEQ ID NO: 240)
MSEASPASGPRHLMDPHIFTSNFNNGIGRHKTYLCYEVERLDNGTSVKMDQHRGFLHNQA
KNLLCGFYGRHAELRFLDLVPSLQLDPAQIYRVTWFISWSPCFSWGCAGEVRAFLQENTHV
RLRIFAARIYDYDPLYKEALQMLRDAGAQVSIMTYDEFKHCWDTFVDHQGCPFQPWDGLD
EHSQALSGRLRAILQNQGN
Human APOBEC3B
(SEQ ID NO: 241)
MNPQIRNPMERMYRDTFYDNFENEPILYGRSYTWLCYEVKIKRGRSNLLWDTGVFRGQVY
FKPQYHAEMCFLSWFCGNQLPAYKCFQITWFVSWTPCPDCVAKLAEFLSEHPNVTLTISAAR
LYYYWERDYRRALCRLSQAGARVTIMDYEEFAYCWENFVYNEGQQFMPWYKFDENYAFL
HRTLKEILRYLMDPDTFTFNFNNDPLVLRRRQTYLCYEVERLDNGTWVLMDQHMGFLCNE
AKNLLCGFYGRHAELRFLDLVPSLQLDPAQIYRVTWFISWSPCFSWGCAGEVRAFLQENTH
VRLRIFAARIYDYDPLYKEALQMLRDAGAQVSIMTYDEFEYCWDTFVYRQGCPFQPWDGL
EEHSQALSGRLRAILQNQGN
Human APOBEC3G
(SEQ ID NO: 242)
MKPHFRNTVERMYRDTFSYNFYNRPILSRRNTVWLCYEVKTKGPSRPPLDAKIFRGQVYSE
LKYHPEMRFFHWFSKWRKLHRDQEYEVTWYISWSPCTKCTRDMATFLAEDPKVTLTIFVA
RLYYFWDPDYQEALRSLCQKRDGPRATMKIMNYDEFQHCWSKFVYSQRELFEPWNNLPK
YYILLHIMLGEILRHSMDPPTFTFNFNNEPWVRGRHETYLCYEVERMHNDTWVLLNQRRGF
LCNQAPHKHGFLEGRHAELCFLDVIPFWKLDLDQDYRVTCFTSWSPCFSCAQEMAKFISKN
KHVSLCIFTARIYDDQGRCQEGLRTLAEAGAKISIMTYSEFKHCWDTFVDHQGCPFQPWDG
LDEHSQDLSGRLRAILQNQEN
Human AID
(SEQ ID NO: 243)
MDSLLMNRRKFLYQFKNVRWAKGRRETYLCYVVKRRDSATSFSLDFGYLRNKNGCHVELL
FLRYISDWDLDPGRCYRVTWFTSWSPCYDCARHVADFLRGNPNLSLRIFTARLYFCEDRKAE
PEGLRRLHRAGVQIAIMTFKDYFYCWNTFVENHERTFKAWEGLHENSVRLSRQLRRILLPLY
EVDDLRDAFRTLGL
Petromyzon marinus CDA1 (pmCDA1, or CDA)
(SEQ ID NO: 244)
MTDAEYVRIHEKLDIYTFKKQFFNNKKSVSHRCYVLFELKRRGERRACFWGYAVNKPQSGT
ERGIHAEIFSIRKVEEYLRDNPGQFTINWYSSWSPCADCAEKILEWYNQELRGNGHTLKIWA
CKLYYEKNARNQIGLWNLRDNGVGLNVMVSEHYQCCRKIFIQSSHNQLNENRWLEKTLKR
AEKRRSELSIMIQVKILHTTKSPAV
evoAPOBEC3A (eA3A)
(SEQ ID NO: 245)
MEASPASGPRHLMDPHIFTSNFNNGIGRHKTYLCYEVERLDNGTSVKMDQHRGFLHGQAK
NLLCGFYGRHAELRFLDLVPSLQLDPAQIYRVTWFISWSPCFSWGCAGEVRAFLQENTHVR
LRIFAARIYDYDPLYKEALQMLRDAGAQVSIMTYDEFKHCWDTFVDHQGCPFQPWDGLDE
HSQALSGRLRAILQNQGN
evoCDA
(SEQ ID NO: 246)
MTDAEYVRIHEKLDIYTFKKQFSNNKKSVSHRCYVLFELKRRGERRACFWGYAVNKPQSGT
ERGIHAEIFSIRKVEEYLRDNPGQFTINWYSSWSPCADCAEKILEWYNQELRGNGHTLKIWV
CKLYYEKNARNQIGLWNLRDNGVGLNVMVSEHYQCCRKIFIQSSHNQLNENRWLEKTLKR
AEKRRSELSIMFQVKILHTTKSPAV
evoAPOBEC1
(SEQ ID NO: 247)
MSSKTGPVAVDPTLRRRIEPHEFEVFFDPRELRKETCLLYEINWGGRHSIWRHTSQNTNKHV
EVNFIEKFTTERYFCPNTRCSITWFLSWSPCGECSRAITEFLSRYPNVTLFIYIARLYHLANPR
NRQGLRDLISSGVTIQIMTEQESGYCWHNFVNYSPSNESHWPRYPHLWVRLYVLELYCIILG
LPPCLNILRRKQSQLTSFTIALQSCHYQRLPPHILWATGLK
YE1
(SEQ ID NO: 248)
MSSETGPVAVDPTLRRRIEPHEFEVFFDPRELRKETCLLYEINWGGRHSIWRHTSQNTNKHV
EVNFIEKFTTERYFCPNTRCSITWFLSYSPCGECSRAITEFLSRYPHVTLFIYIARLYHHADPEN
RQGLRDLISSGVTIQIMTEQESGYCWRNFVNYSPSNEAHWPRYPHLWVRLYVLELYCIILGL
PPCLNILRRKQPQLTFFTIALQSCHYQRLPPHILWATGLK
YE2
(SEQ ID NO: 249)
MSSETGPVAVDPTLRRRIEPHEFEVFFDPRELRKETCLLYEINWGGRHSIWRHTSQNTNKHV
EVNFIEKFTTERYFCPNTRCSITWFLSYSPCGECSRAITEFLSRYPHVTLFIYIARLYHHADPRN
RQGLEDLISSGVTIQIMTEQESGYCWRNFVNYSPSNEAHWPRYPHLWVRLYVLELYCIILGL
PPCLNILRRKQPQLTFFTIALQSCHYQRLPPHILWATGLK
YEE
(SEQ ID NO: 250)
MSSETGPVAVDPTLRRRIEPHEFEVFFDPRELRKETCLLYEINWGGRHSIWRHTSQNTNKHV
EVNFIEKFTTERYFCPNTRCSITWFLSYSPCGECSRAITEFLSRYPHVTLFIYIARLYHHADPEN
RQGLEDLISSGVTIQIMTEQESGYCWRNFVNYSPSNEAHWPRYPHLWVRLYVLELYCIILGL
PPCLNILRRKQPQLTFFTIALQSCHYQRLPPHILWATGLK
EE
(SEQ ID NO: 251)
MSSETGPVAVDPTLRRRIEPHEFEVFFDPRELRKETCLLYEINWGGRHSIWRHTSQNTNKHV
EVNFIEKFTTERYFCPNTRCSITWFLSWSPCGECSRAITEFLSRYPHVTLFIYIARLYHHADPE
NRQGLEDLISSGVTIQIMTEQESGYCWRNFVNYSPSNEAHWPRYPHLWVRLYVLELYCIILG
LPPCLNILRRKQPQLTFFTIALQSCHYQRLPPHILWATGLK
R33A
(SEQ ID NO: 252)
MSSETGPVAVDPTLRRRIEPHEFEVFFDPRELAKETCLLYEINWGGRHSIWRHTSQNTNKHV
EVNFIEKFTTERYFCPNTRCSITWFLSWSPCGECSRAITEFLSRYPHVTLFIYIARLYHHADPR
NRQGLRDLISSGVTIQIMTEQESGYCWRNFVNYSPSNEAHWPRYPHLWVRLYVLELYCIILG
LPPCLNILRRKQPQLTFFTIALQSCHYQRLPPHILWATGLK
R33A + K34A
(SEQ ID NO: 253)
MSSETGPVAVDPTLRRRIEPHEFEVFFDPRELAAETCLLYEINWGGRHSIWRHTSQNTNKHV
EVNFIEKFTTERYFCPNTRCSITWFLSWSPCGECSRAITEFLSRYPHVTLFIYIARLYHHADPR
NRQGLRDLISSGVTIQIMTEQESGYCWRNFVNYSPSNEAHWPRYPHLWVRLYVLELYCIILG
LPPCLNILRRKQPQLTFFTIALQSCHYQRLPPHILWATGLK
AALN
(SEQ ID NO: 254)
MSSETGPVAVDPTLRRRIEPHEFEVFFDPRELAAETCLLYEINWGGRHSIWRHTSQNTNKHV
EVNFIEKFTTERYFCPNTRCSITWFLSWSPCGECSRAITEFLSRYPHVTLFIYIARLYHLANPR
NRQGLRDLISSGVTIQIMTEQESGYCWRNFVNYSPSNEAHWPRYPHLWVRLYVLELYCIILG
LPPCLNILRRKQPQLTFFTIALQSCHYQRLPPHILWATGLK
FERNY
(SEQ ID NO: 255)
MFERNYDPRELRKETYLLYEIKWGKSGKLWRHWCQNNRTQHAEVYFLENIFNARRFNPST
HCSITWYLSWSPCAECSQKIVDFLKEHPNVNLEIYVARLYYHEDERNRQGLRDLVNSGVTIR
IMDLPDYNYCWKTFVSDQGGDEDYWPGHFAPWIKQYSLKL
evoFERNY
(SEQ ID NO: 256)
MFERNYDPRELRKETYLLYEIKWGKSGKLWRHWCQNNRTQHAEVYFLENIFNARRFNPST
HCSITWYLSWSPCAECSQKIVDFLKEHPNVNLEIYVARLYYPENERNRQGLRDLVNSGVTIRI
MDLPDYNYCWKTFVSDQGGDEDYWPGHFAPWIKQYSLKL

### Additional Base Editor Elements

In various embodiments, the base editors disclosed herein further comprise one or more additional elements, e.g., linkers and localization sequences such as nuclear localization sequences (NLSs), nuclear export sequences, UGI domains, and other protein domains.

In some embodiments, the base editors disclosed herein further comprise one or more nuclear localization sequences. In some embodiments, the base editors comprise at least two NLSs. In certain embodiments, the base editors comprise two bipartite NLSs. In some embodiments, the disclosed base editors comprise more than two bipartite NLSs. In embodiments with at least two NLSs, the NLSs can be the same NLSs, or they can be different NLSs. In addition, the NLSs may be expressed as part of a cytosine base editor. The location of the NLS fusion can be at the N-terminus, the C-terminus, or within a sequence of a base editor (e.g., inserted between the napDNAbp domain (e.g., Cas9 domain) and cytidine deaminase domain).

A representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization signals often comprise proline residues. A variety of nuclear localization signals have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated herein by reference. Translocation is currently thought to involve nuclear pore proteins.

The NLSs may be any known NLS in the art. The NLSs may also be any NLSs for nuclear localization discovered in the future. The NLSs also may be any naturally occurring NLS, or any non-naturally occurring NLS (e.g., an NLS with one or more desired mutations).

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines on the protein surface. Different nuclear localized proteins may share the same NLS. An NLS has the opposite function of a nuclear export signal (NES), which targets proteins out of the nucleus. A nuclear localization signal can also target the exterior surface of a cell. Thus, a single nuclear localization signal can direct the entity with which it is associated to the exterior of a cell and to the nucleus of a cell. Such sequences can be of any size and composition, for example, more than 25, 25, 15, 12, 10, 8, 7, 6, 5, or 4 amino acids, but will preferably comprise at least a four to eight amino acid sequence known to function as a nuclear localization signal (NLS).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT Application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 283), MDSLLMNRRKFLYQFKNVRWAKGRRETYLC (SEQ ID NO: 284), KRTADGSEFESPKKKRKV (SEQ ID NO: 285), or KRTADGSEFEPKKKRKV (SEQ ID NO: 286). In other embodiments, the NLS comprises the amino acid sequence: NLSKRPAAIKKAGQAKKKK (SEQ ID NO: 287), PAAKRVKLD (SEQ ID NO: 288), RQRRNELKRSF (SEQ ID NO: 289), or NQSSNFGPMKGGNFGGRSSGPYGGGGQYFAKPRNQGGY (SEQ ID NO: 290). In certain embodiments of the disclosed base editors, an N-terminal NLS comprising KRTADGSEFESPKKKRKV (SEQ ID NO: 285) and a C-terminal NLS comprising or KRTADGSEFEPKKKRKV (SEQ ID NO: 286) is used.

Most NLSs can be classified in three general groups: (i) a monopartite NLS exemplified by the SV40 large T antigen NLS (PKKKRKV (SEQ ID NO: 283)); (ii) a bipartite motif consisting of two basic domains separated by a variable number of spacer amino acids and exemplified by the Xenopus nucleoplasmin NLS (KRXXXXXXXXXXKKKL (SEQ ID NO: 291)); and (iii) noncanonical sequences such as M9 of the hnRNP Al protein, the influenza virus nucleoprotein NLS, and the yeast Gal4 protein NLS (Dingwall and Laskey, Trends Biochem Sci. 1991 December; 16(12):478-81).

Nuclear localization signals appear at various points in the amino acid sequences of proteins. NLSs have been identified at the N-terminus, the C-terminus, and in the central region of proteins. Thus, the specification provides base editors that may be modified with one or more NLSs at the C-terminus, the N-terminus, as well as at in internal region of the base editor. The residues of a longer sequence that do not function as component NLS residues should be selected so as not to interfere, for example tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a base editor to include one or more NLSs. In one aspect, the base editors can be engineered to express a base editor protein that is translationally fused at its N-terminus or its C-terminus (or both) to one or more NLSs, i.e., to form a base editor-NLS fusion construct. In other embodiments, the base editor-encoding nucleotide sequence can be genetically modified to incorporate a reading frame that encodes one or more NLSs in an internal region of the encoded base editor. In addition, the NLSs may include various amino acid linkers or spacer regions encoded between the base editor and the N-terminally, C-terminally, or internally-attached NLS amino acid sequence. Thus, the present disclosure also provides for nucleotide constructs, vectors, and host cells for expressing a base editor and one or more NLSs.

The base editors described herein may also comprise nuclear localization signals which are linked to a base editor through one or more linkers, e.g., polymeric, amino acid, polysaccharide, chemical, or nucleic acid linker element. In certain embodiments, the NLS is linked to a base editor using an XTEN linker, as set forth in SEQ ID NO: 301. The linkers within the contemplated scope of the disclosure are not intended to have any limitations and can be any suitable type of molecule (e.g., polymer, amino acid, polysaccharide, nucleic acid, lipid, or any synthetic chemical linker domain) and be joined to the base editor by any suitable strategy that effectuates forming a bond (e.g., covalent linkage, hydrogen bonding) between the base editor and the one or more NLSs.

In other embodiments, the base editors described herein may comprise one or more uracil glycosylase inhibitor (UGI) domains. In some embodiments, the base editors comprise two UGI domains. The UGI domain refers to a protein that is capable of inhibiting a uracil-DNA glycosylase base-excision repair enzyme.

In some embodiments, a UGI domain comprises a wild-type UGI or a UGI as set forth in SEQ ID NO: 292, or a variant thereof. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 292. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 292. In some embodiments, a UGI comprises an amino acid sequence homologous to the amino acid sequence set forth in SEQ ID NO: 292, or an amino acid sequence homologous to a fragment of the amino acid sequence set forth in SEQ ID NO: 292. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 292. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 292. In some embodiments, the UGI comprises the following amino acid sequence:

>spIP147391UNGI_BPPB2 Uracil-DNA glycosylase inhibitor MTNLSDIIEKETGKQLVIQESILMLPEEVEEVIGNKPESDILVHTAYDESTDENVMLLTSDAPE YKPWALVIQDSNGENKIKML (SEQ ID NO: 292).

The base editors described herein also may include one or more additional elements. In certain embodiments, an additional element may comprise an effector of base repair, such as an inhibitor of base repair.

In some embodiments, the base editors described herein may comprise one or more heterologous protein domains (e.g., about or more than about 1, 2, 3, 4, 5, 6, 7, 8, 9, 10, or more domains in addition to the base editor components). A base editor may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags.

Examples of protein domains that may be fused to a base editor or component thereof (e.g., the napDNAbp domain, the cytidine deaminase domain, or the NLS domain) include, without limitation, epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A base editor may be fused to a gene sequence encoding a protein or a fragment of a protein that binds DNA molecules or binds other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a base editor are described in US Patent Publication No. 2011/0059502, published Mar. 10, 2011, and incorporated herein by reference in its entirety.

The reporter gene sequences that may be used with the base editors, methods and systems disclosed herein include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), HSV thymidine kinase, rpoB, may be introduced into a cell to encode a gene into which a mutation may be introduced that will confer resistance to a particular medium in a growth selection assay for the described system.

Other exemplary features that may be present are tags that are useful for solubilization, purification, or detection of the CBEs. Suitable protein tags provided herein include, but are not limited to, biotin carboxylase carrier protein (BCCP) tags, myc-tags, calmodulin-tags, FLAG-tags, hemagglutinin (HA)-tags, bgh-PolyA tags, polyhistidine tags, and also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the CBE may comprise one or more His tags.

### Linkers

In certain embodiments, linkers may be used to link any of the peptides or peptide domains or domains of the base editor (e.g., a napDNAbp domain covalently linked to a cytidine deaminase domain which is covalently linked to an NLS domain). The base editors described herein may comprise linkers of 32 amino acids and/or 9 amino acids in length. In certain embodiments, the disclosed base editors comprise a first linker of 32 amino acids in length and a second linker of 9 amino acids in length.

As defined above, the term “linker,” as used herein, refers to a chemical group or a molecule linking two molecules or domains, e.g., a napDNAbp binding domain and a cleavage domain of a nuclease. In some embodiments, a linker joins an nCas9 and deaminase domains. Typically, the linker is positioned between, or flanked by, two groups, molecules, or other domains and connected to each one via a covalent bond, thus connecting the two. In some embodiments, the linker is an amino acid or a plurality of amino acids (e.g., a peptide or protein). In some embodiments, the linker is an organic molecule, group, polymer, or chemical domain. Chemical domains include, but are not limited to, disulfide, thermophi, thiol, amide, ester, carbon-carbon bond, carbon-heteroatom bond, urea, carbamate, and azo moieties.

The linker may comprise a peptide or a non-peptide moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the linker is a single atom in length. In certain embodiments, the linker is 32 amino acids, 16 amino acids, and/or 9 amino acids in length. Longer or shorter linkers are also contemplated.

The linker may be as simple as a covalent bond, or it may be a multi-atom linker or polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, polyether, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic domain (e.g., cyclopentane, cyclohexane). In other embodiments, the linker comprises a polyethylene glycol domain (PEG). In other embodiments, the linker comprises amino acids. In certain embodiments, the linker comprises a peptide. In certain embodiments, the linker comprises an aryl or heteroaryl domain. In certain embodiments, the linker is based on a phenyl ring. The linker may included funtionalized domains to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)ₙ (SEQ ID NO: 293), (G)ₙ(SEQ ID NO: 294), (EAAAK)ₙ (SEQ ID NO: 295), (GGS)ₙ (SEQ ID NO: 296), (SGGS)ₙ(SEQ ID NO: 297), (XP). (SEQ ID NO: 298), or any combination thereof, wherein n is independently an integer between 1 and 30, and wherein X is any amino acid. In some embodiments, the linker comprises the amino acid sequence (GGS)ₙ(SEQ ID NO: 299), wherein n is 1, 3, or 7. In some embodiments, the linker comprises the amino acid sequence SGSETPGTSESATPES (SEQ ID NO: 300). In exemplary embodiments, the linker comprises the 32-amino acid sequence SGGSSGGSSGSETPGTSESATPESSGGSSGGS (SEQ ID NO: 301), also known as an XTEN linker. In some embodiments, the linker comprises the 9-amino acid sequence SGGSGGSGGS (SEQ ID NO: 302). In some embodiments, the linker comprises the 4-amino acid sequence SGGS (SEQ ID NO: 303).

In some embodiments, any of the disclosed cytosine base editors comprises the structure [cytidine deaminase domain]-[optional linker sequence]-[dCas9 or Cas9 nickase]-[optional linker sequence], or [dCas9 or Cas9 nickase]-[optional linker sequence]-[cytidine deaminase domain].

### Guide Sequences (e.g., Guide RNAs)

The present disclosure further provides guide RNAs for use in accordance with the disclosed methods of editing and systems and methods for determining off-target effects of base editors. The disclosure provides guide RNAs that are designed to recognize target sequences. Such gRNAs may be designed to have guide sequences (or “spacers”) having complementarity to a protospacer within the target sequence. The disclosure further provides guide RNAs that are designed to recognize sequences other than the target sequences, or off-target sequences. Such gRNAs may be designed to have guide sequences having complementarity to a protospacer within one or more off-target sequences.

Guide RNAs are also provided for use with one or more of the disclosed base editors, e.g., in the disclosed methods of editing a nucleic acid molecule. Such gRNAs may be designed to have guide sequences having complementarity to a protospacer within a target sequence to be edited, and to have backbone sequences that interact specifically with the napDNAbp domains of any of the disclosed base editors, such as Cas9 nickase domains of the disclosed base editors.

In various embodiments, the base editors may be complexed, bound, or otherwise associated with (e.g., via any type of covalent or non-covalent bond) one or more guide sequences. The guide sequence becomes associated or bound to the base editor and directs its localization to a specific target sequence having complementarity to the guide sequence or a portion thereof. The particular design embodiments of a guide sequence will depend upon the nucleotide sequence of a genomic target sequence (i.e., the desired site to be edited) and the type of napDNAbp (e.g., type of Cas9 protein) present in the base editor, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of the napDNAbp (e.g., a Cas9 or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In various embodiments of the assays and systems disclosed herein for measuring off-target frequencies, further provided herein are guide sequences that directs localization of a base editor and/or isolated Cas9 protein to a specific off-target site that is unrelated to a target sequence and has complementarity to the guide sequence or a portion thereof. The particular design embodiments of a guide sequence will depend upon the nucleotide sequence of a genomic off-target site of interest and the type of napDNAbp (e.g., type of Cas9 protein) present in the base editor, among other factors, such as PAM sequence locations, percent G/C content in the target sequence, the degree of microhomology regions, secondary structures, etc. For this purpose, a guide sequence is any polynucleotide sequence having sufficient complementarity with an off-target polynucleotide sequence to hybridize with this sequence and direct sequence-specific binding of the napDNAbp (e.g., a Cas9 or Cas9 variant) to the sequence. In some embodiments, the degree of complementarity between a guide sequence and its corresponding off-target sequence, when optimally aligned using a suitable alignment algorithm, is about or more than about 50%, 60%, 75%, 80%, 85%, 90%, 95%, 97.5%, 99%, or more.

In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, each gRNA comprises a guide sequence of at least 10 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that is complementary to a target sequence (or off-target site).

In some embodiments, a guide sequence is less than about 200, 175, 150, 125, 100, 75, 50, 45, 40, 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence, such as by transfection with vectors encoding the components of a base editor disclosed herein, followed by an assessment of preferential cleavage within the target sequence. Similarly, cleavage of a target polynucleotide sequence may be evaluated in situ by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG (SEQ ID NO: 58) where NNNNNNNNNNNNXGG (N is A, G, T, or C; and X can be anything) (SEQ ID NO: 59) has a single occurrence in the genome. A unique target sequence in a genome may include an S. pyogenes Cas9 target site of the form MMMMMMMMMNNNNNNNNNNNXGG (SEQ ID NO: 60), where NNNNNNNNNNNXGG (N is A, G, T, or C; and X can be anything) (SEQ ID NO: 61) has a single occurrence in the genome. For the S. 187hermophiles CRISPR1 Cas9, a unique target sequence in a genome may include a Cas9 target site of the form MMMMMMMMNNNNNNNNNNNNXXAGAAW (SEQ ID NO: 62) where NNNNNNNNNNNNXXAGAAW (N is A, G, T, or C; X can be anything; and W is A or T) (SEQ ID NO: 63) has a single occurrence in the genome. A unique target sequence in a genome may include an S. 187hermophiles CRISPR 1 Cas9 target site of the form MMMMMMMMMNNNNNNNNNNNXXAGAAW (SEQ ID NO: 64) where NNNNNNNNNNNXXAGAAW (N is A, G, T, or C; X can be anything; and W is A or T) (SEQ ID NO: 65) has a single occurrence in the genome. For the S. pyogenes Cas9, a unique target sequence in a genome may include a Cas9 target site of the form MMMMMMMMNNNNNNNNNNNNXGGXG (SEQ ID NO: 66) where NNNNNNNNNNNNXGGXG (N is A, G, T, or C; and X can be anything) (SEQ ID NO: 67) has a single occurrence in the genome. A unique target sequence in a genome may include an S. pyogenes Cas9 target site of the form MMMMMMMMMNNNNNNNNNNNXGGXG (SEQ ID NO: 68) where NNNNNNNNNNNXGGXG (N is A, G, T, or C; and X can be anything) (SEQ ID NO: 69) has a single occurrence in the genome. In each of these sequences “M” may be A, G, T, or C, and need not be considered in identifying a sequence as unique.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker & Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see, e.g., A. R. Gruber et al., 2008, Cell 106(1): 23-24; and PA Carr & GM Church, 2009, Nature Biotechnology 27(12): 1151-62). Additional algorithms may be found in Chuai, G. et al., DeepCRISPR: optimized CRISPR guide RNA design by deep learning, Genome Biol. 19:80 (2018), and U.S. Application Ser. No. 61/836,080 and U.S. Pat. No. 8,871,445, issued Oct. 28, 2014, the entireties of each of which are incorporated herein by reference.

The disclosed systems and methods for determining off-target effects rely on the use of multiple unique guide RNA sequences, including guide RNA sequences that are engineered to interact specifically with certain napDNAbp proteins, such as Cas9 proteins (see FIG. 2E). Accordingly, guide RNAs are provided for use in accordance with the disclosed eukaryotic cell systems, and for use in accordance with the disclosed prokaryotic cell systems.

The guide sequence of the gRNA is linked to a tracr mate (also known as a “backbone”) sequence which in turn hybridizes to a tracr sequence. A tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In certain embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator: (1) NNNNNNNNgtttttgtactctcaagatttaGAAAtaaatcttgcagaagctacaaagataaggctt catgccgaaatcaacaccctgtcattttatggcagggtgttttcgttatttaaTTTTTT (SEQ ID NO: 71); (2) NNNNNNNNNNNNNNNNNNgtttttgtactctcaGAAAtgcagaagctacaaagataaggcttcatgccgaaatca acaccctgtcattttatggcagggtgttttcgttatttaaTTTTTT (SEQ ID NO: 72); (3) NNNNNNNNNNNNNNNNNNNNgtttttgtactctcaGAAAtgcagaagctacaaagataaggcttcatgccgaaatca acaccctgtcattttatggcagggtgtTTTTT (SEQ ID NO: 73); (4) NNNNNNNNNNNNNNNNNNNNgttttagagctaGAAAtagcaagttaaaataaggctagtccgttatcaacttgaaaa agtggcaccgagtcggtgcTTTTTT (SEQ ID NO: 74); (5) NNNNNNNNNNNNNNNNNNNNgttttagagctaGAAATAGcaagttaaaataaggctagtccgttatcaacttgaa aaagtgTTTTTTT (SEQ ID NO: 75); and (6) NNNNNNNNNNNNNNNNNNNNgttttagagctagAAATAGcaagttaaaataaggctagtccgttatcaTTTTT TTT (SEQ ID NO: 76). In some embodiments, sequences (1) to (3) are used in combination with Cas9 from S. Thermophiles CRISPR1. In some embodiments, sequences (4) to (6) are used in combination with Cas9 from S. pyogenes. In some embodiments, the tracr sequence is a separate transcript from a transcript comprising the tracr mate sequence.

In some embodiments, the guide RNAs for use in accordance with the disclosed methods of editing comprise a backbone structure that is recognized by an S. pyogenes Cas9 protein or domain, such as an SpCas9 domain of the disclosed base editors. The backbone structure recognized by an SpCas9 protein may comprise the sequence 5′-[guide sequence]-guuuuagagcuagaaauagcaaguuaaaauaaggcuaguccguuaucaacuugaaaaaguggcaccgagucggugcuuuuu-3′ (SEQ ID NO: 80), wherein the guide sequence comprises a sequence that is complementary to the protospacer of the target sequence. See U.S. Publication No. 2015-0166981, published Jun. 18, 2015, the disclosure of which is incorporated by reference herein. The guide sequence is typically 20 nucleotides long.

In other embodiments, the guide RNAs for use in accordance with the disclosed methods of editing comprise a backbone structure that is recognized by an S. aureus Cas9 protein. The backbone structure recognized by an SaCas9 protein may comprise the sequence 5′-[guide sequence]-guuuuaguacucuguaaugaaaauuacagaaucuacuaaaacaaggcaaaaugccguguuuaucucgucaacuuguuggcgagau uuuuuu-3′ (SEQ ID NO: 81). In other embodiments, the guide RNAs for use in accordance with the disclosed methods of editing and methods of determining off-target effects in the comprise a backbone structure that is recognized by an S. aureus Cas9 protein, such as an isolated SaCas9 protein.

The sequences of suitable guide RNAs for targeting the disclosed CBEs to specific genomic target sites will be apparent to those of skill in the art based on the present disclosure. Such suitable guide RNA sequences typically comprise guide sequences that are complementary to a nucleic sequence within 50 nucleotides upstream or downstream of the target nucleobase pair to be edited. Some exemplary guide RNA sequences suitable for targeting any of the provided CBEs to specific target sequences are provided herein. Additional guide sequences are well known in the art and may be used with the base editors described herein. Additional exemplary guide sequences are disclosed in, for example, Jinek M., et al., Science 337:816-821(2012); Mali P, Esvelt K M & Church G M (2013) Cas9 as a versatile tool for engineering biology, Nature Methods, 10, 957-963; Li J F et al., (2013) Multiplex and homologous recombination-mediated genome editing in Arabidopsis and Nicotiana benthamiana using guide RNA and Cas9, Nature Biotechnology, 31, 688-691; Hwang, W. Y et al., Efficient genome editing in zebrafish using a CRISPR-Cas system, Nature Biotechnology 31, 227-229 (2013); Cong L et al., (2013) Multiplex genome engineering using CRIPSR/Cas systems, Science, 339, 819-823; Cho S W et al., (2013) Targeted genome engineering in human cells with the Cas9 RNA-guided endonuclease, Nature Biotechnology, 31, 230-232; Jinek, M. et al., RNA-programmed genome editing in human cells, eLife 2, e00471 (2013); Dicarlo, J. E. et al., Genome engineering in Saccharomyces cerevisiae using CRISPR-Cas systems. Nucleic Acid Res. (2013); Briner A E et al., (2014) Guide RNA functional modules direct Cas9 activity and orthogonality, Mol Cell, 56, 333-339, the entire contents of each of which are incorporated herein by reference.

### Methods for Making Base Editors

The disclosure further relates in various aspects to methods of making the disclosed base editors by various modes of manipulation that include, but are not limited to, codon optimization of one or more domains of the disclosed base editors to achieve greater expression levels in a cell, and the use of nuclear localization sequences (NLSs), preferably at least two NLSs, e.g., two bipartite NLSs, to increase the localization of the expressed base editors into a cell nucleus. The base editors contemplated herein can include modifications that result in increased expression, for example, through codon optimization.

In some embodiments, the base editors (or any component thereof) is codon optimized for expression in particular cells, such as eukaryotic cells (e.g. mammalian cells or human cells). The eukaryotic cells may be those of or derived from a particular organism, such as a mammal, including, but not limited to, human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database,” and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a CRISPR enzyme correspond to the most frequently used codon for a particular amino acid. In some embodiments, nucleic acid constructs are codon-optimized for expression in HEK293T cells. In some embodiments, nucleic acid constructs are codon-optimized for expression in mammalian cells. In some embodiments, nucleic acid constructs are codon-optimized for expression in human cells.

In other embodiments, the base editors of the invention have improved expression (as compared to non-modified or state of the art counterpart editors) as a result of ancestral sequence reconstruction analysis. Ancestral sequence reconstruction (ASR) is the process of analyzing modern sequences within an evolutionary/phylogenetic context to infer the ancestral sequences at particular nodes of a tree. Reference is made to Koblan et al., Nat Biotechnol. 2018; 36(9):843-846. These ancient sequences are most often then synthesized, recombinantly expressed in laboratory microorganisms or cell lines, and then characterized to reveal the ancient properties of the extinct biomolecules. This process has produced tremendous insights into the mechanisms of molecular adaptation and functional divergence. Despite such insights, a major criticism of ASR is the general inability to benchmark accuracy of the implemented algorithms. It is difficult to benchmark ASR for many reasons. Notably, genetic material is not preserved in fossils on a long enough time scale to satisfy most ASR studies (many millions to billions of years ago), and it is not yet physically possible to travel back in time to collect samples. Reference can be made to Cai et al., “Reconstruction of ancestral protein sequences and its applications,” BMC Evolutionary Biology 2004, 4:33; and Zakas et al., “Enhancing the pharmaceutical properties of protein drugs by ancestral sequence reconstruction,” Nature Biotechnology, 35-37 (2017), each of which are incorporated herein by reference. Exemplary base editors of the present disclosure that are engineered through ancestral reconstruction include ancestrally reconstructed BE4max base editors (AncBE4max).

There are many software packages available which can perform ancestral state reconstruction. Generally, these software packages have been developed and maintained through the efforts of scientists in related fields and released under free software licenses. The following list is not meant to be a comprehensive itemization of all available packages, but provides a representative sample of the extensive variety of packages that implement methods of ancestral reconstruction with different strengths and features: PAML (Phylogenetic Analysis by Maximum Likelihood, available at abacus.gene.ucl.ac.uk/software/paml.html), BEAST (Bayesian evolutionary analysis by sampling trees, available at www.beast2.org/wiki/index.php/Main_Page), and Diversitree (FitzJohn R G, 2012. Diversitree: comparative phylogenetic analyses of diversification in R. Methods in Ecology and Evolution), and HyPHy (Hypothesis testing using phylogenies, available at hyphy.org/w/index.php/Main_Page).

The above description is meant to be non-limiting with regard to making base editors for increased expression, and thereby increase editing efficiencies.

### High Efficiencies in Genomic Editing

In some embodiments, the target nucleotide sequence is a DNA sequence in a genome, e.g. a eukaryotic genome. In certain embodiments, the target nucleotide sequence is in a mammalian (e.g. a human) genome. In certain embodiments, the target nucleotide sequence is in a human genome. In other embodiments, the target nucleotide sequence is in the genome of a rodent, such as a mouse or rat. In other embodiments, the target nucleotide sequence is in the genome of a domesticated animal, such as a horse, cat, dog, or rabbit. In some embodiments, the target nucleotide sequence is in the genome of an experimental or research animal. In some embodiments, the target nucleotide sequence is in the genome of a plant. In some embodiments, the target nucleotide sequence is in the genome of a microorganism, such as a bacteria.

Some embodiments of the disclosure are based on the recognition that any of the base editors provided herein possess the ability to modify a specific nucleobase while generating a reduced frequency of indels. An “indel”, as used herein, refers to the insertion or deletion of a nucleobase within a nucleic acid. Such insertions or deletions can lead to frame shift mutations within a coding region of a gene. In some embodiments, it is desirable to generate base editors that efficiently modify (e.g., deaminate) a specific nucleotide within a nucleic acid, without generating a large number of insertions or deletions (i.e., indels) in the nucleic acid. In certain embodiments, any of the base editors provided herein are capable of generating a greater proportion of intended modifications (e.g., point mutations) versus indels.

In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method. In some embodiments, to calculate indel frequencies, sequencing reads are scanned for exact matches to two 10-bp sequences that flank both sides of a window in which indels might occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. Indel formation may be measured by techniques known in the art, including high-throughput screening of sequencing reads.

In some embodiments, the base editors provided herein are capable of limiting formation of indels in a region of a nucleic acid. In some embodiments, the region is at a nucleotide targeted by a base editor or a region within 2, 3, 4, 5, 6, 7, 8, 9, 10, 12, 14, 16, 18, 20 or 25 nucleotides of a nucleotide targeted by a base editor. In some embodiments, any of the base editors provided herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. In some embodiments, any of the disclosed base editors are capable of limiting the formation of indels to less than 0.75%, less than 0.6%, less than 0.5%, less than 0.4%, less than 0.3%, or less than 0.2% in the target nucleic acid molecule. In certain embodiments, any of the disclosed base editors provide an indel formation frequency of about 0.5% or less in the target nucleic acid molecule.

The number of indels formed at a nucleic acid region may depend on the amount of time a nucleic acid (e.g., a nucleic acid within the genome of a cell) is exposed to a base editor. In some embodiments, an number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some embodiments of the disclosure are based on the recognition that the formation of indels in a region of a nucleic acid may be limited by nicking the non-edited strand opposite to the strand in which edits are introduced. This nick serves to direct mismatch repair machinery to the non-edited strand, ensuring that the chemically modified nucleobase is not interpreted as a lesion by the machinery. This nick may be created by the use of an nCas9. The methods provided in this disclosure comprise cutting (or nicking) the non-edited strand of the double-stranded DNA, for example, wherein the one strand comprises the T of the target G:C nucleobase pair.

Some embodiments of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations. In some embodiments, an intended mutation is a mutation that is generated by a specific base editor bound to a gRNA, specifically designed to generate the intended mutation. In some embodiments, the intended mutation is intended to correct a mutation associated with a disease, disorder, or condition. In some embodiments, the mutation associated with a disease, disorder, or condition is a thymine (T) to cytosine (C) point mutation. In some embodiments, the mutation associated with a disease, disorder, or condition is an adenine (A) to guanine (G) point mutation.

In some embodiments, the disclosed editing methods result in an actual or average off-target DNA editing frequency of about 2.0% or less, 1.75% or less, 1.5% or less, 1.2% or less, 1% or less, 0.9% or less, 0.8% or less, 0.75% or less, 0.7% or less, 0.65% or less, or 0.6% or less. In some embodiments, the disclosed editing methods result in an actual or average off-target DNA editing frequency of 0.5%, less than 0.5%, less than 0.4%, less than 0.25%, less than 0.2%, less than 0.15%, less than 0.1%, less than 0.05%, or less than 0.025%. In a particular embodiment, the methods result in an actual or average off-target DNA editing frequency of about 0.4% (for instance, methods for evaluating the off-target frequencies of CBEs comprising YE1 deaminase). These off-target editing frequencies may be obtained in sequences having any level of sequence identity to the target sequence. As used herein to refer to off-target DNA editing frequencies, the modifier “average” refers to a mean value over all editing events detected at sites other than a given target nucleobase pair (e.g., as detected by high-throughput sequencing). Exemplary methods of high-throughput sequencing are described in, e.g., Example 3 of this disclosure.

In particular embodiments, the described editing methods generate (or exhibit) an average frequency of off-target editing of less than 1.5%. In some embodiments, the described editing methods generate (or exhibit) an average frequency of off-target editing of less than 1.25%, less than 1.0%, less than 0.75%, or less than 0.5%).

In some embodiments, the disclosed editing methods further result in an actual or average Cas9-independent off-target DNA editing frequency of about 2.0% or less, 1.75% or less, 1.5% or less, 1.2% or less, 1% or less, 0.9% or less, 0.8% or less, 0.75% or less, 0.7% or less, 0.65% or less, or 0.6% or less. In other words, the disclosed editing methods further result in an actual or average off-target DNA editing frequency of about 2.0% or less, 1.75% or less, 1.5% or less, 1.2% or less, 1% or less, 0.9% or less, 0.8% or less, 0.75% or less, 0.7% or less, 0.65% or less, or 0.6% or less in sequences having 60% or less sequence identity to the target sequence. In some embodiments, the disclosed editing methods result in an actual or average off-target DNA editing frequency of 0.5%, less than 0.5%, less than 0.4%, less than 0.25%, less than 0.2%, less than 0.15%, less than 0.1%, less than 0.05%, or less than 0.025%, in sequences having 60% or less sequence identity to the target sequence. In some embodiments, these editing frequencies are obtained in sequences comprising protospacer sequences having 5, 6, 7, 8, 9, 10, or more than 10 mismatches relative to protospacer sequence of the target sequence. In a particular embodiment, the methods result in an actual or average Cas9-independent off-target DNA editing frequency of 0.4% or less.

In various embodiments, the disclosed editing methods result in an on-target DNA base editing efficiency of at least about 35%, 40%, 50%, 60%, 70%, 80%, 85%, 90%, 95%, 98%, or 99% at the target nucleobase pair. The step of contacting may result in in a DNA base editing efficiency of at least about 60%, 61%, 62%, 63%, 64%, 65%, 66%, 67%, 68%, 69%, 70%, 71%, 72%, 73%, 74%, or 75%. In particular, the step of contacting results in on-target base editing efficiencies of greater than 75%. In certain embodiments, base editing efficiencies of 99% may be realized.

In some embodiments, the method results in less than 5%, or less than 10%, indel formation in the nucleic acid. In some embodiments, the method results in less than 2%, 1%, 0.5%, 0.2%, or 0.1% indel formation. In some embodiments, at least 5% of the intended base pairs in a population of cells or in tissues in vivo are edited. In some embodiments, at least 10%, 15%, 20%, 25%, 30%, 35%, 40%, 45%, or 50% of the intended base pairs in a population of cells or in tissues in vivo are edited.

In some embodiments, the ratio of intended products to unintended products in the target nucleotide is at least 2:1, 5:1, 10:1, 20:1, 30:1, 40:1, 50:1, 60:1, 70:1, 80:1, 90:1, 100:1, 200:1, or more. In some embodiments, the ratio of intended point mutation to indel formation is greater than 1:1, 10:1, 50:1, 100:1, 500:1, or 1000:1, or more.

In some embodiments, the intended mutation is a cytosine (C) to thymine (T) point mutation within the coding region of a gene. In some embodiments, the intended mutation is a guanine (G) to adenine (A) point mutation within the coding region of a gene. In some embodiments, the intended mutation is a point mutation that generates a stop codon, for example, a premature stop codon within the coding region of a gene.

### Vectors

Some aspects of this disclosure relate to polynucleotides and vector constructs for producing the disclosed base editors. Some aspects of this disclosure relate to cells (e.g., host cells) comprising the base editors, cells comprising the disclosed polynucleotides, and cells comprising the disclosed vectors.

Some aspects of this disclosure relate to methods of engineering and producing one or more components of the base editors disclosed herein. Further aspects relate to methods of engineering the base editors and complexes comprising one or more guide nucleic acid molecules (e.g., Cas9 guide RNAs) and a base editor, as provided herein. In addition, some embodiments of the disclosure provide methods of using the base editors for editing a target nucleic acid molecule (e.g., a genomic sequence, a cDNA sequence, or a viral DNA sequence).

In certain embodiments, methods of manufacturing the base editors for use in the methods of DNA editing, methods of treatment, on-target and off-target editing assays, pharmaceutical compositions, and kits disclosed herein comprise the use of recombinant protein expression methodologies and techniques known to those of skill in the art.

Several embodiments of the making and using of the base editors of the invention relate to vector systems comprising one or more vectors, or vectors as such. Likewise, several embodiments of the methods for determining off-target effects of base editors relate to vector systems comprising one or more vectors. Vectors may be designed to clone and/or express the base editors as disclosed herein. Vectors may also be designed to clone and/or express one ore more gRNAs having complementarity to the target sequence, as disclosed herein. Vectors may also be designed to transfect the base editors and gRNAs of the disclosure into one or more cells, e.g., a target diseased eukaryotic cell for treatment with the base editor systems and methods disclosed herein. Exemplary vectors utilized in the methods and systems provided in the Examples of the present disclosure comprise the BE4-P2A-GFP, YE1-P2A-GFP, YE1-NG-P2A-GFP, YE1-BE4-CP1028-P2A-GFP, and Cas9(D10A)-P2A-GFP plasmid vectors.

Vectors can be designed for expression of base editor transcripts (e.g., nucleic acid transcripts, proteins, or enzymes) in prokaryotic or eukaryotic cells. For example, base editor transcripts can be expressed in bacterial cells such as Escherichia coli, insect cells (using baculovirus expression vectors), yeast cells, plant cells, or mammalian cells. Suitable host cells are discussed further in Goeddel, Gene Expression Technology: Methods In Enzymology 185, Academic Press. San Diego, Calif. (1990). Alternatively, expression vectors encoding one or more base editors described herein can be transcribed and translated in vitro, for example, using T7 promoter regulatory sequences and T7 polymerase.

Vectors may be introduced and propagated in a prokaryotic cells. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion proteins or non-fusion proteins.

Fusion expression vectors also may be used to express the base editors of the disclosure. Such vectors generally add a number of amino acids to a protein encoded therein, such as to the amino terminus of the recombinant protein. Such fusion vectors may serve one or more purposes, such as: (i) to increase expression of a recombinant protein; (ii) to increase the solubility of a recombinant protein; and (iii) to aid in the purification of a recombinant protein by acting as a ligand in affinity purification. Often, in fusion expression vectors, a proteolytic cleavage site is introduced at the junction of the fusion domain and the recombinant protein to enable separation of the recombinant protein from the fusion domain subsequent to purification of the base editor. Such enzymes, and their cognate recognition sequences, include Factor Xa, thrombin and enterokinase. Exemplary fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods In Enzymology 185, Academic Press, San Diego, Calif. (1990) 60-89).

In some embodiments, a vector is a yeast expression vector for expressing the base editors described herein. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

### Methods of Editing a Target Nucleobase Pair, Methods of Treatment, and Uses for the Base Editors

Some embodiments of the disclosure provide methods for editing a target nucleobase pair in a nucleic acid (e.g., in a double-stranded DNA sequence). In some embodiments, the methods comprise the steps of contacting a target region of a nucleic acid (e.g., a double-stranded DNA sequence) with a complex comprising a base editor and a guide nucleic acid (e.g., gRNA), wherein the target region comprises a targeted nucleobase pair. As a result of certain embodiments of these methods, strand separation of said target region is induced, a first nucleobase of said target nucleobase pair in a single strand of the target region is converted to a second nucleobase, and no more than one strand of said target region is cut (or nicked), wherein a third nucleobase complementary to the first nucleobase base is replaced by a fourth nucleobase complementary to the second nucleobase.

The disclosed methods may further comprise cutting (or nicking) no more than one strand of the target region, whereby a third nucleobase complementary to the first nucleobase is replaced by a fourth nucleobase complementary to the second nucleobase. In certain embodiments, the first nucleobase is a cytosine (of the target C:G nucleobase pair). In some embodiments, the second nucleobase is a uracil (i.e., the C is converted to U). In some embodiments, the third nucleobase is a guanine (of the target C:G base pair), and the fourth nucleobase is a adenine. In some embodiments, the second nucleobase is replaced with a fifth nucleobase (thymine) that is complementary to the fourth nucleobase, thereby generating an intended edited base pair (e.g., a T:A pair).

In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited base pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair is within the target window. In some embodiments, the target window comprises the intended edited base pair. In some embodiments, the method is performed using any of the base editors provided herein. In some embodiments, a target window is a editing window. In some embodiments, the target window is an editing window of 2-20 nucleotides, preferably 2-10 or 2-8 nucleotides.

In another embodiment, the disclosure provides editing methods comprising contacting a DNA, or RNA molecule with any of the base editors provided herein, and with at least one guide nucleic acid (e.g., guide RNA), wherein the guide nucleic acid, (e.g., guide RNA) is about 15-100 nucleotides long and comprises a sequence of at least 10 contiguous nucleotides that is complementary to a target sequence. In some embodiments, the 3′ end of the target sequence is immediately adjacent to a canonical PAM sequence (NGG). In some embodiments, the 3′ end of the target sequence is not immediately adjacent to a canonical PAM sequence (NGG). In some embodiments, the 3′ end of the target sequence is immediately adjacent to an AGC, GAG, TTT, GTG, or CAA sequence.

In some embodiments, the target nucleic acid sequence comprises a sequence associated with a disease, disorder, or condition. In some embodiments, the target nucleic acid sequence comprises a point mutation associated with a disease, disorder, or condition. In some embodiments, the activity of the base editor or the complex with a gRNA, results in a correction of the point mutation. In some embodiments, the target nucleic acid sequence comprises a T→C point mutation associated with a disease, disorder, or condition, and wherein the conversion of the mutant C to a T results in a sequence that is not associated with a disease, disorder, or condition. The target sequence may comprise an A→G point mutation associated with a disease, disorder, or condition, and wherein the conversion of the mutant G to a A results in a sequence that is not associated with a disease, disorder, or condition. In some embodiments, the target nucleic acid sequence encodes a protein, and the point mutation is in a codon and results in a change in the amino acid encoded by the mutant codon as compared to the wild-type codon. In some embodiments, the conversion of the mutant C (or mutant G) results in a change of the amino acid encoded by the mutant codon. In some embodiments, the conversion of the mutant C (or mutant G) results in the codon encoding the wild-type amino acid. In some embodiments, the contacting is in vivo in a subject. In some embodiments, the subject has or has been diagnosed with a disease, disorder, or condition.

In some embodiments, the disclosed cytosine base editors are used to introduce a point mutation into a nucleic acid by deaminating a target C nucleobase to a uracil nucleobase. In some embodiments, the deamination of the target C and substitution of the uracil intermediate to a thymine (T) nucleobase results in the correction of a genetic defect, e.g., in the correction of a point mutation that leads to a loss of function in a gene product. In some embodiments, the genetic defect is associated with a disease, disorder, or condition, e.g., a lysosomal storage disorder or a metabolic disease, such as, for example, type I diabetes. In some embodiments, the methods provided herein are used to introduce a deactivating point mutation into a gene or allele that encodes a gene product that is associated with a disease, disorder, or condition. For example, in some embodiments, methods are provided herein that employ a base editor to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein.

In some embodiments, the methods provided herein are intended to restore the function of a dysfunctional gene via genome editing. The base editors provided herein can be validated for gene editing-based human therapeutics in vitro, e.g., by correcting a disease-associated mutation in human cell culture. It will be understood by the skilled artisan that the base editors provided herein, e.g., the base editors comprising a nucleic acid programmable DNA binding protein (e.g., Cas9) and a nucleotide modification domain can be used to correct any single point T to G or A to C mutation. Deamination of a C that is base-paired with the mutant G, followed by a round of replication, may correct the mutation.

The successful correction of point mutations in disease-associated genes and alleles opens up new strategies for gene correction with applications in therapeutics and basic research. Site-specific single-base modification systems like the disclosed fusions of a nucleic acid programmable DNA binding protein and a cytidine deaminase domain also have applications in “reverse” gene therapy, where certain gene functions are purposely suppressed or abolished. In these cases, site-specifically mutating residues that lead to inactivating mutations in a protein, or mutations that inhibit function of the protein can be used to abolish or inhibit protein function.

### Methods of Treatment

The present disclosure provides methods for the treatment of a subject diagnosed with a disease associated with or caused by a point mutation that can be corrected by a base editor (e.g., a cytosine base editor) provided herein. For example, in some embodiments, a method is provided that comprises administering to a subject having such a disease, e.g., a cancer associated with a point mutation as described above, an effective amount of a base editor and a gRNA that forms a complex with the CBE, that corrects the point mutation or introduces a deactivating mutation into a disease-associated gene. In some embodiments, a method is provided that comprises administering to a subject having such a disease, e.g., a cancer associated with a point mutation as described above, an effective amount of a base editor-gRNA complex that corrects the point mutation or introduces a deactivating mutation into a disease-associated gene. Further provided herein are methods comprising administering to a subject one or more vectors that contains a nucleotide sequence that expresses the base editor and gRNA that forms a complex with the base editor.

In some embodiments, the disease is a proliferative disease. In some embodiments, the disease is a genetic disease. In some embodiments, the disease is a neoplastic disease. In some embodiments, the disease is a metabolic disease. Other diseases that can be treated by correcting a point mutation or introducing a deactivating mutation into a disease-associated gene will be known to those of skill in the art, and the disclosure is not limited in this respect.

The present disclosure provides methods for the treatment of additional diseases or disorders, e.g., diseases or disorders that are associated or caused by a point mutation that can be corrected by base editing. Some such diseases are described herein, and additional suitable diseases that can be treated with the strategies and CBEs provided herein will be apparent to those of skill in the art based on the present disclosure. Exemplary suitable diseases and disorders are listed below. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues. Exemplary suitable diseases and disorders include, without limitation: Non-Bruton type Agammaglobulinemia, Hypomyelinating Leukodystrophy, 21-hydroxylase deficiency, familial Breast-ovarian cancer, Immunodeficiency with basal ganglia calcification, Congenital myasthenic syndrome, Shprintzen-Goldberg syndrome, Peroxisome biogenesis disorder, Nephronophthisis, autosomal recessive early-onset, digenic, PINK1/DJ1 Parkinson disease, Cerebral visual impairment and intellectual disability, Neurodevelopmental disorder with or without anomalies of the brain, eye, or heart, Immunodeficiency, Leber congenital amaurosis, Amyotrophic lateral sclerosis type 10, Motor neuron disease, Malignant melanoma of skin, Focal cortical dysplasia type II, papillary Renal cell carcinoma, Glioblastoma, Colorectal Neoplasms, Uterine cervical neoplasms, sporadic Papillary renal cell carcinoma, Malignant neoplasm of body of uterus, Kidney Carcinoma, Neoplasm of the breast, Glioblastoma, Smith-Kingsmore syndrome, Homocysteinemia due to MTHFR deficiency, type 2A2A Charcot-Marie-Tooth disease, Bartter syndrome type 3, Cataract, multiple types, Gastrointestinal stroma tumor, Paragangliomas, Pheochromocytoma, Hereditary cancer-predisposing syndrome, Paragangliomas, Hereditary cancer-predisposing syndrome, Gastrointestinal stroma tumor, Paragangliomas, Pheochromocytoma, Hereditary Paraganglioma-Pheochromocytoma Syndromes, Hereditary cancer-predisposing syndrome, Gastrointestinal stroma tumor, Paraganglioma and gastric stromal sarcoma, Uncombable hair syndrome, Parkinson disease, autosomal recessive early-onset, Childhood hypophosphatasia, Odontohypophosphatasia, Takenouchi-Kosaki syndrome, C1q deficiency, Prostate cancer/brain cancer susceptibility, UDPglucose-4-epimerase deficiency, Deficiency of hydroxymethylglutaryl-CoA lyase, Fucosidosis, nonsyndromic cleft palate, Van der Woude syndrome, autosomal recessive Hypercholesterolemia, Eichsfeld type congenital muscular dystrophy, autosomal dominant Mental retardation, Hyperphosphatasia with mental retardation syndrome, Hyperphosphatasia-intellectual disability syndrome, Obesity, mild, early-onset, Ectodermal dysplasia, hypohidrotic/hair/tooth/nail type, Dystonia, torsion, autosomal recessive, Reticular dysgenesis, Erythrokeratodermia variabilis et progressiva, Corneal dystrophy, Fuchs endothelial, Corneal dystrophy, posterior polymorphous, Hereditary neutrophilia, Ceroid lipofuscinosis neuronal, Neuronal ceroid lipofuscinosis, Lethal tight skin contracture syndrome, DFNA 2 Nonsyndromic Hearing Loss, Osteogenesis imperfecta type 8, GLUT1 deficiency syndrome, autosomal recessive, Glucose transporter type 1 deficiency syndrome, Congenital amegakaryocytic thrombocytopenia, Myelofibrosis with myeloid metaplasia, somatic, Myelofibrosis with myeloid metaplasia, Thrombocythemia, somatic, Hematologic neoplasm, Early infantile epileptic encephalopathy, Mental retardation, autosomal recessive, Familial porphyria cutanea tarda, MYH-associated polyposis, Hereditary cancer-predisposing syndrome, MUTYH-associated polyposis, Hereditary cancer-predisposing syndrome, Methylmalonic acidemia with homocystinuria, Methylmalonic aciduria and homocystinuria, cblC type, digenic, Muscle eye brain disease, Congenital Muscular Dystrophy, alpha-dystroglycan related, Limb-Girdle Muscular Dystrophy, Recessive, Muscle eye brain disease, Congenital muscular dystrophy-dystroglycanopathy with brain and eye anomalies, type A3, Adenocarcinoma of the colon, Congenital primary aphakia, Hepatic failure, early-onset, and neurologic disorder due to cytochrome C oxidase deficiency, Carnitine palmitoyltransferase II deficiency, infantile, Carnitine palmitoyltransferase II deficiency, myopathic, stress-induced, Carnitine palmitoyltransferase II deficiency, Carnitine palmitoyltransferase II deficiency, myopathic, stress-induced, Sensorineural deafness with mild renal dysfunction, Bartter syndrome type 4, Hypercholesterolemia, autosomal dominant, Low density lipoprotein cholesterol level quantitative trait locus, Familial hypercholesterolemia, Hypocholesterolemia, Hypercholesterolemia, autosomal dominant, Familial hypercholesterolemia, Low density lipoprotein cholesterol level quantitative trait locus, Hypocholesterolemia, Lattice corneal dystrophy Type III, Epileptic encephalopathy, early infantile, Hypobetalipoproteinemia, familial, Congenital disorder of glycosylation type 1t, Leber congenital amaurosis, Retinitis pigmentosa, Medium-chain acyl-coenzyme A dehydrogenase deficiency, Dilated cardiomyopathy 1CC, Venous malformation, Aase syndrome, Stargardt disease, Cone-rod dystrophy, Retinitis pigmentosa, Stargardt disease, Congenital stationary night blindness, Retinal dystrophy, Nonsyndromic cleft lip with or without cleft palate, Glycogen storage disease type III, Glycogen storage disease IIIa, Intermediate maple syrup urine disease type 2, Maple syrup urine disease, Chorea, childhood-onset, with psychomotor retardation, Marshall syndrome, Stickler syndrome, type 2, Marshall/Stickler syndrome, Chudley-McCullough syndrome, Auriculocondylar syndrome, Pontocerebellar hypoplasia, type 9, Epileptic encephalopathy, early infantile, Spinocerebellar ataxia, Muscle AMP deaminase deficiency, Congenital giant melanocytic nevus, Liver cancer, Chronic lymphocytic leukemia, Neurocutaneous melanosis, Malignant melanoma of skin, Multiple myeloma, Neuroblastoma, Lung adenocarcinoma, Non-small cell lung cancer, Acute myeloid leukemia, Renal cell carcinoma, papillary, Neoplasm of brain, Cutaneous melanoma, Glioblastoma, Hepatocellular carcinoma, Transitional cell carcinoma of the bladder, Colorectal Neoplasms, Nasopharyngeal Neoplasms, Adrenocortical carcinoma, Adenocarcinoma of stomach, Ovarian Serous Cystadenocarcinoma, Malignant neoplasm of body of uterus, RAS Inhibitor response, Malignant lymphoma, non-Hodgkin, Medulloblastoma, Malignant melanoma of skin, Multiple myeloma, Acute myeloid leukemia, Myelodysplastic syndrome, Cutaneous melanoma, Transitional cell carcinoma of the bladder, Neoplasm, Colorectal Neoplasms, Adenocarcinoma of stomach, Cutaneous melanoma, Malignant melanoma of skin, Multiple myeloma, Acute myeloid leukemia, Noonan syndrome, Myelodysplastic syndrome, Cutaneous melanoma, Colorectal Neoplasms, Adenocarcinoma of stomach, Malignant neoplasm of body of uterus, Malignant melanoma of skin, Multiple myeloma, Non-small cell lung cancer, Acute myeloid leukemia, Myelodysplastic syndrome, Cutaneous melanoma, Colorectal Neoplasms, Adenocarcinoma of stomach, Malignant neoplasm of body of uterus, Secondary hypothyroidism, Cardiovascular phenotype, Neurodevelopmental disorder, mitochondrial, with abnormal movements and lactic acidosis, with or without seizures, beta-Hydroxysteroid dehydrogenase deficiency, Hajdu-Cheney syndrome, Hemochromatosis type 2A, Hemochromatosis type 1, Atrial fibrillation, familial, Nager syndrome, Severe congenital neutropenia, autosomal recessive, Retinitis pigmentosa, Neurodevelopmental disorder with microcephaly, hypotonia, and variable brain anomalies, Abnormality of brain morphology, Neurodevelopmental disorder with microcephaly, hypotonia, and variable brain anomalies, Paget disease of bone, White-sutton syndrome, Ichthyosis vulgaris, Dermatitis, atopic, Ichthyosis vulgaris, Mental retardation, autosomal dominant, Nemaline myopathy, Congenital myopathy with fiber type disproportion, Epilepsy, nocturnal frontal lobe, type 3, Aicardi-Goutieres syndrome, Gaucher disease, perinatal lethal, Gaucher's disease, type 1, Subacute neuronopathic Gaucher's disease, Pyruvate kinase deficiency of red cells, Mental retardation, autosomal dominant, myopathy, mitochondrial, and ataxia, Grange syndrome, Charcot-Marie-Tooth disease, type 2, Familial partial lipodystrophy, Hutchinson-Gilford progeria syndrome, childhood-onset, Charcot-Marie-Tooth disease, Charcot-Marie-Tooth disease, type 2, Familial partial lipodystrophy, Benign scapuloperoneal muscular dystrophy with cardiomyopathy, Mandibuloacral dysostosis, Dilated cardiomyopathy, Encephalopathy, progressive, early-onset, with brain edema and/or leukoencephalopathy, Infantile encephalopathy, Hereditary insensitivity to pain with anhidrosis, Familial medullary thyroid carcinoma, Hereditary insensitivity to pain with anhidrosis Spherocytosis, type 3, autosomal recessive, Spherocytosis, Recessive, Elliptocytosis, Hereditary pyropoikilocytosis, Elliptocytosis, Spherocytosis, Recessive, Enlarged vestibular aqueduct, Alternating hemiplegia of childhood, Autoimmune interstitial lung, joint, and kidney disease, Mitochondrial complex I deficiency, Charcot-Marie-Tooth disease, demyelinating, type 1b, Charcot-Marie-Tooth disease, type I, Roussy-Lévy syndrome, Neuropathy, congenital hypomyelinating, autosomal dominant, Charcot-Marie-Tooth disease, demyelinating, type 1b, Charcot-Marie-Tooth disease type 2J, Charcot-Marie-Tooth disease dominant intermediate, Charcot-Marie-Tooth disease, type I, Gastrointestinal stroma tumor, Paragangliomas, Hereditary cancer-predisposing syndrome, Achromatopsia, Thrombophilia due to activated protein C resistance, Geroderma osteodysplastica, Trimethylaminuria, FMO3 activity, decreased, Trimethylaminuria, Primary open angle glaucoma juvenile onset, Glaucoma, open angle, digenic, Glaucoma, primary congenital, digenic, MYOC-Related Disorders, Leukoencephalopathy with Brainstem and Spinal Cord Involvement and Lactate Elevation, Antithrombin III deficiency, Antithrombin deficiency, Antithrombin III deficiency, Hereditary nephrotic syndrome, Nephrotic syndrome, idiopathic, steroid-resistant, Pituitary hormone deficiency, combined, Glutamine deficiency, congenital, Prostate cancer, hereditary, Junctional epidermolysis bullosa gravis of Herlitz, Hyperparathyroidism, Factor H deficiency, Basal laminar drusen, CFHR5 deficiency, Factor XIII subunit B deficiency, Primary autosomal recessive microcephaly 5, Macular dystrophy, Leber congenital amaurosis, Retinitis pigmentosa, Leber congenital amaurosis, Macular dystrophy, Acute myeloid leukemia with maturation, Microcephaly, primary, autosomal recessive, Hypokalemic periodic paralysis, Left ventricular noncompaction, Familial hypertrophic cardiomyopathy, Left ventricular noncompaction, Familial restrictive cardiomyopathy, Cardiovascular phenotype, Renal dysplasia, Amelogenesis imperfecta, type IA, Popliteal pterygium syndrome, Van der Woude syndrome, Zimmermann-Laband syndrome, Leber congenital amaurosis, Stromme syndrome, Ciliary dyskinesia, primary, Usher syndrome, type 2A, Retinitis pigmentosa, Usher syndrome, Usher syndrome, type 2A, Retinal dystrophy, USH2A-Related Disorders, Usher syndrome, Blindness, Rod-cone dystrophy, Pigmentary retinopathy, Abnormal macular morphology, Retinal pigment epithelial atrophy, Loeys-Dietz syndrome, Holt-Oram syndrome, Cardiovascular phenotype, Martsolf syndrome, Warburg micro syndrome, Skraban-Deardorff syndrome, Coenzyme Q10 deficiency, primary, Multiple mitochondrial dysfunctions syndrome, Nemaline myopathy, Myopathy, scapulohumeroperoneal, Nemaline myopathy, autosomal dominant or recessive, Myopathy, actin, congenital, with cores, Cardioencephalomyopathy, fatal infantile, due to cytochrome c oxidase deficiency, Chédiak-Higashi syndrome, Familial hypertrophic cardiomyopathy, Methylcobalamin deficiency, cblG type, Catecholaminergic polymorphic ventricular tachycardia, Catecholaminergic polymorphic ventricular tachycardia type 1, Catecholaminergic polymorphic ventricular tachycardia, Tooth agenesis, selective, Multiple cutaneous leiomyomas, Fumarase deficiency, Hereditary cancer-predisposing syndrome, Mental retardation, autosomal dominant, Diamond-Blackfan anemia, Maturity-onset diabetes of the young, type 7, Myoglobinuria, acute recurrent, autosomal recessive, Feingold syndrome, Cranioectodermal dysplasia, Short rib polydactyly syndrome, Jeune thoracic dystrophy, Short-rib thoracic dysplasia without polydactyly, Short-rib thoracic dysplasia with polydactyly, digenic, Multiple epiphyseal dysplasia, Familial hypobetalipoproteinemia, Familial hypercholesterolemia, Hypercholesterolemia, autosomal dominant, type B, Hypobetalipoproteinemia, familial, Hypobetalipoproteinemia, familial, Proopiomelanocortin deficiency, Acute myeloid leukemia, Shashi-Pena syndrome, Primary pulmonary hypertension 4, Navajo neurohepatopathy, Retinitis pigmentosa, Retinitis pigmentosa, Neuroblastoma, Neuroblastoma, Lung adenocarcinoma, Neuroblastoma, Non-small cell lung cancer, Benign Soft Tissue Neoplasm of Uncertain Differentiation, 3-Oxo-5 alpha-steroid delta 4-dehydrogenase deficiency, Spastic paraplegia, autosomal dominant, Glaucoma, primary congenital, Gingival fibromatosis, Noonan syndrome, Noonan syndrome, Rasopathy, Noonan syndrome, Short-rib thoracic dysplasia with polydactyly, Sitosterolemia, Cystinuria, Holoprosencephaly, Single median maxillary incisor, Schizencephaly, Erythrocytosis, familial, Factor v and factor viii, combined deficiency of, Multiple gastrointestinal atresias, Lynch syndrome, Lynch syndrome, Hereditary cancer-predisposing syndrome, Lynch syndrome, Hereditary nonpolyposis colon cancer, Lynch syndrome, Hereditary cancer-predisposing syndrome, Hereditary nonpolyposis colon cancer, Lynch syndrome, Hereditary cancer-predisposing syndrome, Hereditary nonpolyposis colorectal cancer type 5, Lynch syndrome, Hereditary cancer-predisposing syndrome, Colorectal cancer, nonpolyposis, Hereditary nonpolyposis colon cancer, Leydig hypoplasia, type I, Leydig cell agenesis, Ovarian dysgenesis 1, Ovarian hyperstimulation syndrome, Combined oxidative phosphorylation deficiency, Intellectual developmental disorder with persistence of fetal hemoglobin, Bardet-Biedl syndrome, Multiple mitochondrial dysfunctions syndrome, Miyoshi muscular dystrophy, Limb-girdle muscular dystrophy, type 2B, Limb-girdle muscular dystrophy, type 2B, Miyoshi muscular dystrophy, Limb-girdle muscular dystrophy, type 2B, Dysferlinopathy, Limb-girdle muscular dystrophy, type 2B, Miyoshi muscular dystrophy, Limb-girdle muscular dystrophy, type 2B, Radiohumeral fusions with other skeletal and craniofacial anomalies, Sepiapterin reductase deficiency, Alstrom syndrome, Microcephaly-capillary malformation syndrome, Visceral myopathy, Chronic intestinal pseudoobstruction, Progressive external ophthalmoplegia with mitochondrial DNA deletions, autosomal recessive, Mitochondrial DNA-depletion syndrome, hepatocerebral, Congenital disorder of glycosylation type 2B, Vitamin k-dependent clotting factors, combined deficiency of, Surfactant metabolism dysfunction, pulmonary, Wolcott-Rallison dysplasia, Pheochromocytoma, Hereditary cancer-predisposing syndrome, Retinitis pigmentosa, Cone-rod dystrophy amelogenesis imperfecta, Cd8 deficiency, familial, Severe combined immunodeficiency, atypical, Achromatopsia, Monochromacy, Ectodermal dysplasia, hypohidrotic/hair/tooth type, autosomal dominant, Autosomal recessive hypohidrotic ectodermal dysplasia syndrome, Autosomal dominant hypohidrotic ectodermal dysplasia, Colorectal cancer with chromosomal instability, Retinitis pigmentosa, Osteomyelitis, sterile multifocal, with periostitis and pustulosis, Hypochromic microcytic anemia with iron overload, Culler-Jones syndrome, Autosomal recessive centronuclear myopathy, Thrombophilia, hereditary, due to protein C deficiency, autosomal dominant, Congenital disorders of glycosylation type II, Congenital disorder of glycosylation, type IIo, Warburg micro syndrome, Hypomyelination with brainstem and spinal cord involvement and leg spasticity, Warts, hypogammaglobulinemia, infections, and myelokathexis, Congenital NAD deficiency disorder, Vertebral, cardiac, renal, and limb defects syndrome, Mowat-Wilson syndrome, Homocystinuria, cblD type, variant 1, Nemaline myopathy, Nemaline myopathy, Idiopathic generalized epilepsy, Epilepsy, idiopathic generalized, Juvenile myoclonic epilepsy, Episodic ataxia, type 5, Progressive myositis ossificans, Amelogenesis imperfecta, type IH, Benign familial neonatal-infantile seizures, Early infantile epileptic encephalopathy, Episodic ataxia, Early infantile epileptic encephalopathy, Seizures, Vertigo, Benign familial neonatal-infantile seizures, Mental retardation, autosomal dominant, Tumoral calcinosis, familial, hyperphosphatemic, Short rib-polydactyly syndrome, Majewski type, Severe myoclonic epilepsy in infancy, Generalized epilepsy with febrile seizures plus, type 2, Severe myoclonic epilepsy in infancy, Seizures, Delayed speech and language development, Early infantile epileptic encephalopathy, Severe myoclonic epilepsy in infancy, Familial hemiplegic migraine type 3, Paroxysmal extreme pain disorder, Hereditary sensory and autonomic neuropathy type IIA, Generalized epilepsy with febrile seizures plus, type 7, Rolandic epilepsy, Small fiber neuropathy, Primary erythromelalgia, Hereditary sensory and autonomic neuropathy type IIA, Generalized epilepsy with febrile seizures plus, type 7, Inherited Erythromelalgia, Primary erythromelalgia, Indifference to pain, congenital, autosomal recessive, Febrile seizures, familial, 3b, Benign recurrent intrahepatic cholestasis, Progressive familial intrahepatic cholestasis, Myasthenic syndrome, slow-channel congenital, Lethal multiple pterygium syndrome, Duane syndrome type 2, Synpolydactyly, Brachydactyly-syndactyly-oligodactyly syndrome, Brachydactyl-syndactyly-oligodactyly syndrome (1 patient), immunodeficiency, developmental delay, and hypohomocysteinemia, Hereditary myopathy with early respiratory failure, Familial dilated cardiomyopathy, Dilated cardiomyopathy, Primary dilated cardiomyopathy, Limb-girdle muscular dystrophy, type 2J, Primary dilated cardiomyopathy, Familial dilated cardiomyopathy, Familial hypertrophic cardiomyopathy, Diabetes mellitus type 2, Ehlers-Danlos syndrome, type 4, Cardiovascular phenotype, Ehlers-Danlos syndrome, type 2, Ehlers-Danlos syndrome, classic type, Hemochromatosis type 4, Immunodeficiency, Mycobacterial and viral infections, susceptibility to, autosomal recessive, Immunodeficiency, Mental retardation, autosomal recessive, Acute myeloid leukemia, Myelodysplastic syndrome, Myelodysplastic syndrome progressed to acute myeloid leukemia, Mitochondrial complex I deficiency, Joubert syndrome, Infantile-onset ascending hereditary spastic paralysis, ALS2-Related Disorders, Amyotrophic lateral sclerosis type 2, Pulmonary venoocclusive disease, Primary pulmonary hypertension, Autoimmune lymphoproliferatiVe syndrome, type V, Aculeiform cataract, Congenital cataract, Cataract, coppock-like, Liver cancer, Medulloblastoma, Malignant melanoma of skin, Multiple myeloma, Lung adenocarcinoma, Acute myeloid leukemia, Myelodysplastic syndrome, Neoplasm of brain, Neoplasm of the breast, Glioblastoma, Hepatocellular carcinoma, Transitional cell carcinoma of the bladder, Brainstem glioma, Colorectal Neoplasms, Adenoid cystic carcinoma, Adenocarcinoma of prostate, Hypotonia, infantile, with psychomotor retardation and characteristic facies, Congenital hyperammonemia, type I, Hereditary cancer-predisposing syndrome, Familial cancer of breast, Hereditary cancer-predisposing syndrome, Hereditary cancer-predisposing syndrome, Spondylometaphyseal dysplasia—Sutcliffe type, Spondylometaphyseal dysplasia, Short stature, Focal segmental glomerulosclerosis, Microcephaly, Small for gestational age, Disproportionate short-trunk short stature, Decreased body weight, Atrioventricular canal defect, Congenital microcephaly, Steroid-resistant nephrotic syndrome, Schimke immunoosseous dysplasia, Short stature, Focal segmental glomerulosclerosis, Microcephaly, Small for gestational age, Disproportionate short-trunk short stature, Decreased body weight, Atrioventricular canal defect, Congenital microcephaly, Steroid-resistant nephrotic syndrome, Gracile syndrome, Cholestanol storage disease, Odontoonychodermal dysplasia, Schopf-Schulz-Passarge syndrome, Tooth agenesis, selective, Type A1 brachydactyly, Dyschromatosis universalis hereditaria, Charcot-Marie-Tooth disease, axonal, type 2T, Myopathy, centronuclear, Three M syndrome, Waardenburg syndrome type 1, Alport syndrome, autosomal recessive, Benign familial hematuria, Basal ganglia disease, biotin-responsive, ARMC9-related Joubert syndrome, ARMC9-related Joubert syndrome, Jourbert syndrome, Arthrogryposis, distal, type 5d, Microphthalmia, isolated, Myasthenic syndrome, congenital, fast-channel, Congenital myasthenic syndrome, fast-channel, Oguchi's disease, Crigler Najjar syndrome, type 1, Crigler-Najjar syndrome, type II, Crigler-Najjar syndrome, Crigler-Najjar syndrome, type II, Gilbert's syndrome, Crigler Najjar syndrome, type 1, Hyperbilirubinemia, Ullrich congenital muscular dystrophy, Bethlem myopathy, Ullrich congenital muscular dystrophy, Bethlem myopathy, Primary hyperoxaluria, type I, D-2-hydroxyglutaric aciduria, Sideroblastic anemia with B-cell immunodeficiency, periodic fevers, and developmental delay, Multiple sulfatase deficiency, Gillespie syndrome, Limb-girdle muscular dystrophy, type 1C, Rippling muscle disease, Familial partial lipodystrophy, Severe congenital neutropenia, autosomal recessive, Severe congenital neutropenia, Von Hippel-Lindau syndrome, Hereditary cancer-predisposing syndrome, Erythrocytosis, familial, Von Hippel-Lindau syndrome, Erythrocytosis, familial, Von Hippel-Lindau syndrome, Renal cell carcinoma, papillary, Metabolic syndrome, susceptibility to, Obesity, age at onset of, Morbid obesity, Noonan syndrome, Rasopathy, Xeroderma pigmentosum, group C, Endplate acetylcholinesterase deficiency, Biotinidase deficiency, Thyroid hormone resistance, generalized, autosomal dominant, Thyroid hormone resistance, selective pituitary, Microphthalmia, syndromic, Congenital disorder of deglycosylation, Cardiovascular phenotype, Loeys-Dietz syndrome, Thoracic aortic aneurysm and aortic dissection, Congenital disorder of glycosylation type 1×, Mucopolysaccharidosis, MPS-IV-B, Osteogenesis imperfecta type 7, Lynch syndrome I, Hereditary cancer-predisposing syndrome, Turcot syndrome, Hereditary nonpolyposis colon cancer, Atrial fibrillation, Atrial fibrillation, familial, Atrial fibrillation, Brugada syndrome, Congenital long QT syndrome, Cardiac arrhythmia, Sudden infant death syndrome, Long qt syndrome, acquired, susceptibility to, Long QT syndrome, Romano-Ward syndrome, Brugada syndrome, Sick sinus syndrome, Progressive familial heart block, Cardiovascular phenotype, Paroxysmal familial ventricular fibrillation, Dilated Cardiomyopathy, Dominant, Long QT syndrome, Congenital long QT syndrome, Cardiac conduction defect, nonprogressive, Cardiac conduction defect, nonspecific, Brugada syndrome, Asplenia, isolated congenital, Liver cancer, Medulloblastoma, Malignant melanoma of skin, Pilomatrixoma, Hepatoblastoma, Hepatocellular carcinoma, Transitional cell carcinoma of the bladder, Uterine cervical neoplasms, Craniopharyngioma, Adenocarcinoma of stomach, Malignant neoplasm of body of uterus, Adenocarcinoma of prostate, Liver cancer, Medulloblastoma, Malignant melanoma of skin, Pilomatrixoma, Lung adenocarcinoma, Carcinoma of colon, Endometrial neoplasm, Hepatocellular carcinoma, Pancreatic adenocarcinoma, Transitional cell carcinoma of the bladder, Carcinoma of esophagus, Colorectal Neoplasms, Adenocarcinoma of stomach, Malignant neoplasm of body of uterus, Adenocarcinoma of prostate, Liver cancer, Medulloblastoma, Malignant melanoma of skin, Squamous cell carcinoma of the head and neck, Malignant tumor of prostate, Lung adenocarcinoma, Hepatoblastoma, Cutaneous melanoma, Hepatocellular carcinoma, Craniopharyngioma, Adrenocortical carcinoma, Adenocarcinoma of stomach, Malignant neoplasm of body of uterus, Liver cancer, Medulloblastoma, Lung adenocarcinoma, Neoplasm of stomach, Cutaneous melanoma, Hepatocellular carcinoma, Transitional cell carcinoma of the bladder, Carcinoma of esophagus, Uterine cervical neoplasms, Adenocarcinoma of stomach, Malignant neoplasm of body of uterus, Adenocarcinoma of prostate, Liver cancer, Malignant melanoma of skin, Lung adenocarcinoma, Cutaneous melanoma, Hepatocellular carcinoma, Transitional cell carcinoma of the bladder, Colorectal Neoplasms, Adrenocortical carcinoma, Malignant neoplasm of body of uterus, Adenocarcinoma of prostate, Nemaline myopathy, Spinocerebellar ataxia, autosomal recessive, Perrault syndrome, Hydrops, lactic acidosis, and sideroblastic anemia, Perrault syndrome, Bardet-Biedl syndrome, Bardet-Biedl syndrome, Failure of tooth eruption, primary, Gray platelet syndrome, Pretibial epidermolysis bullosa, Epidermolysis bullosa pruriginosa, autosomal dominant, Recessive dystrophic epidermolysis bullosa, Microcephaly, progressive, with seizures and cerebral and cerebellar atrophy, Epileptic encephalopathy, Nephrotic syndrome, type 5, with or without ocular abnormalities, Muscular dystrophy-dystroglycanopathy (congenital with brain and eye anomalies), type a, Tumor susceptibility linked to germline BAP1 mutations, Dilated cardiomyopathy 1Z, Dilated cardiomyopathy 1S, Familial hypertrophic cardiomyopathy, Cardiovascular phenotype, Familial hypertrophic cardiomyopathy, Hypogonadotropic hypogonadism with anosmia, Atelosteogenesis type 1, Atelosteogenesis type 3, Spondylocarpotarsal synostosis syndrome, Nemaline myopathy, Mental retardation with language impairment and with or without autistic features, Glycogen storage disease, type IV, Glycogen storage disease IV, congenital neuromuscular, Glycogen storage disease, type IV, Frontotemporal Dementia, Chromosome 3-Linked, Amyotrophic lateral sclerosis, Pituitary hormone deficiency, combined 1, Joubert syndrome, Neuropathy, hereditary motor and sensory, Okinawa type, Macular dystrophy, vitelliform, Retinitis pigmentosa, Combined oxidative phosphorylation deficiency, Spermatogenic failure, epileptic encephalopathy, infantile or early childhood, Alkaptonuria, Senior-Loken syndrome, Leber congenital amaurosis, Nephronophthisis, congenital deafness, Hypocalciuric hypercalcemia, familial, type 1, Neonatal severe hyperparathyroidism, Hypocalcemia, autosomal dominant, Hypocalciuric hypercalcemia, familial, type 1, Neonatal severe hyperparathyroidism, Hypocalciuric hypercalcemia, familial, type 1, Hypocalcemia, autosomal dominant, Hypocalcemia, autosomal dominant, with bartter syndrome, Dyskinesia, familial, with facial myokymia, Visceral myopathy, Lymphedema, primary, with myelodysplasia, Dendritic cell, monocyte, B lymphocyte, and natural killer lymphocyte deficiency, Acyl-CoA dehydrogenase family, member, deficiency of, Retinitis pigmentosa, Retinitis pigmentosa, autosomal recessive, Congenital stationary night blindness, autosomal dominant, Familial benign pemphigus, Epileptic encephalopathy, early infantile, Adolescent nephronophthisis, Primary hypertrophic osteoarthropathy, autosomal recessive, Myopathy, myofibrillar, Propionyl-CoA carboxylase deficiency, Blepharophimosis, ptosis, and epicanthus inversus, Seckel syndrome, Bruck syndrome, Craniosynostosis, Deficiency of ferroxidase, Usher syndrome, type 3A, Usher syndrome, type 3A, Retinitis pigmentosam, Deficiency of butyrylcholine esterase, BCHE, fluoride, Retinitis pigmentosa, Fanconi-Bickel syndrome, Short stature, idiopathic, autosomal, Liver cancer, Malignant melanoma of skin, Squamous cell carcinoma of the head and neck, Small cell lung cancer, Lung adenocarcinoma, Squamous cell lung carcinoma, Renal cell carcinoma, papillary, Neoplasm of brain, Neoplasm of the breast, Glioblastoma, Hepatocellular carcinoma, Pancreatic adenocarcinoma, Transitional cell carcinoma of the bladder, Brainstem glioma, Carcinoma of esophagus, PIK3CA related overgrowth spectrum, Colorectal Neoplasms, Uterine cervical neoplasms, Papillary renal cell carcinoma, sporadic, Nasopharyngeal Neoplasms, Adenocarcinoma of stomach, Ovarian Serous Cystadenocarcinoma, Malignant neoplasm of body of uterus, Adenocarcinoma of prostate, Uterine Carcinosarcoma, Carcinoma of gallbladder, Lung cancer, Medulloblastoma, Malignant melanoma of skin, Squamous cell carcinoma of the head and neck, Malignant tumor of prostate, Ovarian epithelial cancer, Carcinoma of colon, Neoplasm of brain, Neoplasm of the breast, Glioblastoma, Transitional cell carcinoma of the bladder, PIK3CA related overgrowth spectrum, Ovarian Neoplasms, Colorectal Neoplasms, Uterine cervical neoplasms, Adenocarcinoma of stomach, Malignant neoplasm of body of uterus, Adenocarcinoma of prostate, Uterine Carcinosarcoma, Cowden syndrome, PIK3CA related overgrowth spectrum, Colorectal Neoplasms, Ciliary dyskinesia, Ciliary dyskinesia, primary, Microphthalmia syndromic, Methylcrotonyl-CoA carboxylase deficiency, Epidermolysis bullosa simplex, Koebner type, Epidermolysis bullosa simplex, generalized, with scarring and hair loss, Leukoencephalopathy with vanishing white matter, Congenital disorder of glycosylation type 1D, Woolly hair, autosomal recessive, with or without hypotrichosis, Membranous cataract, Myopia, high, with cataract and vitreoretinal degeneration, Primary hypomagnesemia, Dominant hereditary optic atrophy, Autosomal dominant optic atrophy plus syndrome, Dominant hereditary optic atrophy, Abortive cerebellar ataxia, Dominant hereditary optic atrophy, Retinitis pigmentosa, Retinal dystrophy, Congenital stationary night blindness, autosomal dominant, Retinitis pigmentosa, Retinitis pigmentosa, Epileptic encephalopathy, early infantile, Abnormality of brain morphology, Dysostosis multiplex, Mucopolysaccharidosis type I, Hypochondroplasia, Thanatophoric dysplasia type 1, Epidermal nevus, Bladder carcinoma, Achondroplasia, Crouzon syndrome with acanthosis nigricans, Craniosynostosis, Carcinoma, Thanatophoric dysplasia type 1, Achondroplasia, Hypochondroplasia, Craniosynostosis, Camptodactyly, tall stature, and hearing loss syndrome, Hypochondroplasia, Thanatophoric dysplasia type 1, Craniosynostosis, Fibrous dysplasia of jaw, Myasthenia, limb-girdle, familial, Selective tooth agenesis, Orofacial cleft, Hypoplastic enamel-onycholysis-hypohidrosis syndrome, Jeune thoracic dystrophy, Ellis-van Creveld Syndrome, Short rib-polydactyly syndrome, Majewski type, Chondroectodermal dysplasia, Short rib-polydactyly syndrome, Majewski type, Diabetes mellitus type 2, Diabetes mellitus and insipidus with optic atrophy and deafness, Wolfram syndrome, Joubert syndrome, Coach syndrome, Retinitis pigmentosa, Cone-rod dystrophy, Retinal dystrophy, Spastic paraplegia, autosomal recessive, Epileptic encephalopathy, early infantile, Retinitis pigmentosa, Limb-girdle muscular dystrophy, type 2E, Gastrointestinal stroma tumor, Gastrointestinal stromal tumor, familial, Cutaneous mastocytosis, Gastrointestinal stroma tumor, Cutaneous melanoma, Gastrointestinal stroma tumor, Acute myeloid leukemia, Hematologic neoplasm, Cutaneous melanoma, Congenital disorder of glycosylation type 1Q, Hypogonadotropic hypogonadism with or without anosmia, Epilepsy, progressive myoclonic, with or without renal failure, Cryptophthalmos syndrome, Hyaline fibromatosis syndrome, Deafness, autosomal dominant nonsyndromic sensorineural, with dentinogenesis imperfecta, Dentinogenesis imperfecta—Shield's type II, Dentinogenesis imperfecta—Shield's type III, Deafness, autosomal dominant nonsyndromic sensorineural, with dentinogenesis imperfecta, Basan syndrome, Adermatoglyphia, Type A2 brachydactyly, Acromesomelic dysplasia, Demirhan type, Fibular hypoplasia and complex brachydactyly, Brachydactyly, type al, Abetalipoproteinaemia, SLC39A8 deficiency, congenital disorder of glycosylation, type IIn, Beta-D-mannosidosis, Sudden cardiac failure, infantile, Deficiency of 3-hydroxyacyl-CoA dehydrogenase, Hyperinsulinemic hypoglycemia, familial, Fibrosis of extraocular muscles, congenital, Cardiac arrhythmia, Cardiac arrhythmia, ankyrin B-related, Long QT syndrome, Cardiovascular phenotype, Cardiac arrhythmia, Cardiac arrhythmia, ankyrin B-related, Long QT syndrome, Arrhythmia, Cardiovascular phenotype, Bardet-Biedl syndrome, Van Maldergem syndrome, short-rib thoracic dysplasia with polydactyly, Ceroid lipofuscinosis neuronal, Macular dystrophy with central cone involvement, Ceroid lipofuscinosis neuronal, Methylmalonic aciduria cb1A type, Pseudohypoaldosteronism type 1 autosomal dominant, Pseudohypoaldosteronism, Common variable immunodeficiency, with autoimmunity, Afibrinogenemia, congenital, Familial visceral amyloidosis, Ostertag type, Hypodysfibrinogenemia, congenital, Afibrinogenemia, congenital, Glutaric acidemia IIC, Glutaric aciduria, type 2, Short rib-polydactyly syndrome, Majewski type, Dilated cardiomyopathy 1A, Limb-girdle muscular dystrophy, type 2S, Mitochondrial myopathy, Myopia, Mitochondrial DNA depletion syndrome (cardiomyopathic type), autosomal recessive, Progressive sensorineural hearing impairment, Hypertrophic cardiomyopathy, Left ventricular hypertrophy, Vertigo, Abnormality of mitochondrial metabolism, Mitochondrial respiratory chain defects, Bietti crystalline corneoretinal dystrophy, Corneal Dystrophy, Recessive, Bietti crystalline corneoretinal dystrophy, Hereditary factor XI deficiency disease, Mitochondrial complex II deficiency, Paragangliomas, Hereditary cancer-predisposing syndrome, Mitochondrial complex II deficiency, Dyskeratosis congenita autosomal dominant, Ciliary dyskinesia, Mental retardation, autosomal dominant, Chondrocalcinosis, Oculocutaneous albinism type 4, Inherited bone marrow failure syndrome, Bone marrow failure syndrome, Cornelia de Lange syndrome, Joubert syndrome, Orofaciodigital syndrome, Complement component deficiency, C7 and C6 deficiency, combined subtotal, Succinyl-CoA acetoacetate transferase deficiency, Laron syndrome with undetectable serum GH-binding protein, Laron-type isolated somatotropin defect, Levy-Hollister syndrome, Molybdenum cofactor deficiency, complementation group B, Distal hereditary motor neuronopathy type 2C, Kartagener syndrome, Acrodysostosis, with or without hormone resistance, UV-sensitive syndrome, Cockayne syndrome type A, Retinitis pigmentosa with or without skeletal anomalies, Immunodeficiency, Kugelberg-Welander disease, Werdnig-Hoffmann disease, 3-methylcrotonyl CoA carboxylase deficiency, Striatal degeneration, autosomal dominant, Hermansky Pudlak syndrome, Mucopolysaccharidosis, type vi, intermediate, Short stature, microcephaly, and endocrine dysfunction, Wagner syndrome, Basal cell carcinoma, somatic, Capillary malformation-arteriovenous malformation, Usher syndrome, type 2C, Febrile seizures, familial, Bosch-Boonstra-Schaaf optic atrophy syndrome, Proprotein convertase deficiency, Familial adenomatous polyposis, Familial colorectal cancer, Familial adenomatous polyposis, Familial adenomatous polyposis, Hereditary cancer-predisposing syndrome, Familial adenomatous polyposis, Colorectal cancer, susceptibility to, Familial adenomatous polyposis, Hereditary cancer-predisposing syndrome, Familial adenomatous polyposis, Hereditary cancer-predisposing syndrome, Familial adenomatous polyposis, Hereditary cancer-predisposing syndrome, Familial adenomatous polyposis, Anencephalus, Aortic aneurysm, familial thoracic, Pyridoxine-dependent epilepsy, Seizures, Ventriculomegaly, Pyridoxine-dependent epilepsy, Myopathy, areflexia, respiratory distress, and dysphagia, early-onset, Congenital contractural arachnodactyly, Neuromyotonia and axonal neuropathy, autosomal recessive, Renal carnitine transport defect, Hereditary cancer-predisposing syndrome, Chylomicron retention disease, Groenouw corneal dystrophy type I, Reis-Bucklers' corneal dystrophy, Lattice corneal dystrophy type 3A, Lattice corneal dystrophy Type I, Pseudohypoaldosteronism, type 2, Pseudohypoaldosteronism type 2D, Myotilinopathy, Charcot-Marie-Tooth disease, axonal, type 2w, Leber congenital amaurosis, Retinitis pigmentosa, Diastrophic dysplasia, de la Chapelle dysplasia, Achondrogenesis, type IB, Multiple epiphyseal dysplasia, Diastrophic dysplasia, Hereditary diffuse leukoencephalopathy with spheroids, Infantile myofibromatosis, Mental retardation, autosomal recessive, Tay-Sachs disease, variant AB, Hyperglycinuria, Iminoglycinuria, digenic, Hyperekplexia hereditary, epileptic encephalopathy, early infantile, Autosomal recessive congenital ichthyosis, Congenital ichthyosiform erythroderma, Epilepsy, childhood absence, Familial febrile seizures, Leukodystrophy, hypomyelinating, Atrial septal defect with or without atrioventricular conduction defects, Ventricular septal defect, Primary dilated cardiomyopathy, Atrial septal defect, Ventricular fibrillation, Noncompaction cardiomyopathy, Abnormality of cardiovascular system morphology, Malformation of the heart and great vessels, Cardiovascular phenotype, Congenital heart disease, Atrial septal defect with or without atrioventricular conduction defects, Hypothyroidism, congenital, nongoitrous, Cardiovascular phenotype, Congenital heart disease, Craniosynostosis, Lewy body dementia, Sotos syndrome, Hypercalcemia, infantile, Hereditary angioneurotic edema with normal C1 esterase inhibitor activity, Hereditary angioneurotic edema, Acute myeloid leukemia, Myelodysplasia, Ehlers-Danlos syndrome progeroid type, Axenfeld-Rieger syndrome type 3, Polymicrogyria, asymmetric, Combined oxidative phosphorylation deficiency, Combined oxidative phosphorylation deficiency, Factor XIII subunit A deficiency, Cardiovascular phenotype, Bicuspid aortic valve, Arrhythmia, Sudden cardiac death, Ventricular fibrillation, Aortic dilatation, Bicuspid aortic valve, Branchiooculofacial syndrome, Hypoparathyroidism familial isolated, Auriculocondylar syndrome, Lafora disease, Hemochromatosis type 1, Transient neonatal diabetes mellitus, Michelin-tire baby, Combined oxidative phosphorylation deficiency, Peeling skin syndrome, Thrombocytopenia, anemia, and myelofibrosis, Premature ovarian failure, Sialidosis type I, 21-hydroxylase deficiency, Adenoma, cortisol-producing, Carcinoma, adrenocortical, androgen-secreting, Nakajo syndrome, Otospondylomegaepiphyseal dysplasia, Nonsyndromic Deafness, Mental retardation, autosomal dominant, Leber congenital amaurosis, Polycystic lipomembranous osteodysplasia with sclerosing leukoencephalopathy, Macular dystrophy, vitelliform, adult-onset, Retinitis pigmentosa, Choroidal dystrophy, central areolar, Glycine N-methyltransferase deficiency, Heimler syndrome, Three M syndrome, Xeroderma pigmentosum, variant type, Jaberi-Elahi syndrome, Ciliary dyskinesia, Platelet-activating factor acetylhydrolase deficiency, Methylmalonic aciduria due to methylmalonyl-CoA mutase deficiency, methylmalonic aciduria, mut(−) type, Methylmalonic aciduria due to methylmalonyl-CoA mutase deficiency, methylmalonic aciduria, mut(0) type, Methylmalonic acidemia, Methylmalonic aciduria due to methylmalonyl-CoA mutase deficiency, methylmalonic aciduria, mut(−) type Rh-null, regulator type, Rh-mod syndrome, Char syndrome, Autosomal recessive polycystic kidney disease, Polycystic kidney dysplasia, Autosomal recessive polycystic kidney disease, Spinocerebellar ataxia, Retinitis pigmentosa, Retinitis pigmentosa, mental retardation, autosomal dominant, Hydatidiform mole, recurrent, Deafness, autosomal dominant, Macular dystrophy, vitelliform, developmental delay, intellectual disability, obesity, and dysmorphic features, Leber congenital amaurosis, Maple syrup urine disease, Immunodeficiency, Hyper-IgE syndrome, Calcification of joints and arteries, Spinocerebellar ataxia, autosomal recessive, Forney Robinson Pascoe syndrome, Mitochondrial DNA depletion syndrome (encephalomyopathic type), North Carolina macular dystrophy, Spastic paraplegia and psychomotor retardation with or without seizures, Osteopetrosis, autosomal recessive, Amyotrophic lateral sclerosis type, Progressive pseudorheumatoid dysplasia, Metaphyseal chondrodysplasia, Schmid type, Ovarian dysgenesis, Alopecia congenita keratosis palmoplantaris, Oculodentodigital dysplasia, Merosin deficient congenital muscular dystrophy, Laminin alpha 2-related dystrophy, Merosin deficient congenital muscular dystrophy, Arginase deficiency, Arterial calcification of infancy, Hypophosphatemic rickets, autosomal recessive, Arterial calcification of infancy, Hypophosphatemic Rickets, Recessive, Arterial calcification of infancy, Joubert syndrome, Leber congenital amaurosis, Disseminated atypical mycobacterial infection, neurodegeneration with brain iron accumulation, Mental retardation, autosomal dominant, Congenital heart defects, multiple types, Mitochondrial diseases, Combined oxidative phosphorylation deficiency, Mitochondrial diseases, Estrogen resistance, Neoplasm of the breast, Spinocerebellar ataxia, autosomal recessive, Liver cancer, Hepatocellular carcinoma, Plasminogen deficiency, type I, Dysplasminogenemia, Plasminogen deficiency, type I, Parkinson disease, Dentin dysplasia, type I, with extreme microdontia and misshapen teeth, Ciliary dyskinesia, Spondylocostal dysostosis, Baraitser-Winter syndrome, Hereditary cancer-predisposing syndrome, Hereditary nonpolyposis colon cancer, Lynch syndrome, Neurodevelopmental abnormality, leukodystrophy, hypomyelinating, Congenital muscular dystrophy-dystroglycanopathy with brain and eye anomalies, type A7, Congenital muscular dystrophy-dystroglycanopathy with brain and eye anomalies, type A7, Muscular dystrophy-dystroglycanopathy (limb-girdle), type c, Congenital muscular dystrophy-dystroglycanopathy with brain and eye anomalies, type A7, Saethre-Chotzen syndrome, Ciliary dyskinesia, primary, Hypomyelination and Congenital Cataract, microtia without hearing impairment, Microtia, hearing impairment, and cleft palate, Isolated growth hormone deficiency type 1B, Uridine 5-prime monophosphate hydrolase deficiency, hemolytic anemia due to, Bardet-Biedl syndrome, Focal segmental glomerulosclerosis, Wilms tumor and radial bilateral aplasia, Pallister-Hall syndrome, Greig cephalopolysyndactyly syndrome, Pallister-Hall syndrome, Pallister-Hall syndrome, Hyperbiliverdinemia, Ehlers-Danlos syndrome, classic-like, Permanent neonatal diabetes mellitus, Maturity-onset diabetes of the young, type 2, Immunodeficiency, common variable, Cowden syndrome, Lung adenocarcinoma, Non-small cell lung cancer, Nonsmall cell lung cancer, response to tyrosine kinase inhibitor in, somatic, Glioblastoma, Non-small cell lung cancer, Squamous cell lung carcinoma, Carcinoma of esophagus, Non-small cell lung cancer, Mucopolysaccharidosis type VII, Argininosuccinate lyase deficiency, Epilepsy, progressive myoclonic, Shwachman syndrome, Disordered steroidogenesis due to cytochrome p450 oxidoreductase deficiency, Charcot-Marie-Tooth disease, Charcot-Marie-Tooth disease type 2F, Cholestasis, intrahepatic, of pregnancy, Progressive familial intrahepatic cholestasis, Progressive familial intrahepatic cholestasis, Intrahepatic cholestasis, Colchicine resistance, Cerebral cavernous malformation, Cerebral cavernous malformation, Cerebral cavernous malformations, Zellweger syndrome, Deafness enamel hypoplasia nail defects, Myelocerebellar disorder, COL1A2-Related Disorder, Ehlers-Danlos syndrome, classic type, Osteogenesis imperfecta type I, Osteogenesis imperfecta type III, Osteogenesis imperfecta type I, Osteogenesis imperfecta, recessive perinatal lethal, Ehlers-Danlos syndrome, classic type, Osteogenesis imperfecta type I, Osteogenesis imperfecta with normal sclerae, dominant form, Osteogenesis imperfecta type III, Osteogenesis imperfecta with normal sclerae, dominant form, Osteogenesis imperfecta type III, Ehlers-Danlos syndrome, autosomal recessive, cardiac valvular form, Neonatal intrahepatic cholestasis caused by citrin deficiency, Citrullinemia type II, Split-hand/foot malformation, Asparagine synthetase deficiency, Epilepsy, familial temporal lobe, Lissencephaly, Epilepsy, familial temporal lobe, Rolandic epilepsy, Epilepsy, familial temporal lobe, Enlarged vestibular aqueduct, Pendred's syndrome, Pendred's syndrome, Enlarged vestibular aqueduct, Pendred's syndrome, SLC26A4-Related Disorders, Enlarged vestibular aqueduct, Pendred's syndrome, Enlarged vestibular aqueduct, Congenital secretory diarrhea, chloride type, Maple syrup urine disease, type 3, DLD-Related Disorders, Lissencephaly, Lipodystrophy, congenital generalized, type 3, Renal cell carcinoma, papillary, Cystic fibrosis, Hereditary pancreatitis, Cystic fibrosis, Hereditary pancreatitis, ataluren response—Efficacy, Persistent hyperplastic primary vitreous, autosomal recessive, Atrophia bulborum hereditaria, Exudative vitreoretinopathy, Leptin dysfunction, Myofibrillar myopathy, filamin C-related, Myopathy, distal, Cardiomyopathy, familial hypertrophic, Dilated Cardiomyopathy, Dominant, Dilated Cardiomyopathy, Dominant, Basal cell carcinoma, somatic, Ghosal hematodiaphyseal syndrome, Multiple myeloma, Lung adenocarcinoma, Rasopathy, Glioblastoma, Transitional cell carcinoma of the bladder, Cardio-facio-cutaneous syndrome, Malignant melanoma of skin, Multiple myeloma, Lung adenocarcinoma, Non-small cell lung cancer, Squamous cell lung carcinoma, Squamous cell carcinoma of the skin, Transitional cell carcinoma of the bladder, Neoplasm, Colorectal Neoplasms, Adenocarcinoma of prostate, Lung cancer, Malignant melanoma of skin, Multiple myeloma, Squamous cell carcinoma of the head and neck, Lung adenocarcinoma, Non-small cell lung cancer, Squamous cell lung carcinoma, Colorectal Neoplasms, Non-small cell lung cancer, Rasopathy, Neoplasm of the breast, Neoplasm, Carcinoma of colon, Noonan syndrome, Cataract and cardiomyopathy, Myotonia congenital, Congenital myotonia, autosomal recessive form, Premature ovarian failure, Cortical dysplasia-focal epilepsy syndrome, Rolandic epilepsy, Pitt-Hopkins-like syndrome, Rolandic epilepsy, Long QT syndrome, Congenital long QT syndrome, Short QT syndrome, Cardiovascular phenotype, Long QT syndrome, Glaucoma, open angle, F, Glycogen storage disease of heart, lethal congenital, Familial hypertrophic cardiomyopathy, Primary familial hypertrophic cardiomyopathy, Holoprosencephaly, Currarino triad, Limb-girdle muscular dystrophy, type 1E, Neuronal ceroid lipofuscinosis, Maturity-onset diabetes of the young, type 11, Congenital heart disease, Atrial septal defect, Congenital heart disease, Atrial septal defect, Tetralogy of Fallot, Ventricular septal defect, Atrioventricular septal defect, Idiopathic transverse myelitis, Jankovic Rivera syndrome, Farber disease, Hyperlipoproteinemia, type I, Hyperlipoproteinemia, type I, lipoprotein lipase (Olbia), Surfactant metabolism dysfunction, pulmonary, Osteogenesis imperfecta, type xiii, Hypermanganesemia with dystonia, Charcot-Marie-Tooth disease, demyelinating, type 1f, Charcot-Marie-Tooth disease type 2E, Charcot-Marie-Tooth disease, demyelinating, type 1f, Trichothiodystrophy 6, nonphotosensitive, Cholesterol monooxygenase (side-chain cleaving) deficiency, Kallmann syndrome, Hartsfield syndrome, Medulloblastoma, Neuroblastoma, Encephalocraniocutaneous lipomatosis, Astrocytoma, Brainstem glioma, Adenocarcinoma of stomach, Rosette-forming glioneuronal tumor, Hypogonadotropic hypogonadism with anosmia, Spherocytosis type 1, Mental retardation, autosomal dominant, Idiopathic basal ganglia calcification, Basal ganglia calcification, idiopathic, Dystonia, torsion, Mucopolysaccharidosis, MPS-III-C, Retinitis pigmentosa, Mucopolysaccharidosis, MPS-III-C, Vesicoureteral reflux, CHARGE association, Ataxia with vitamin E deficiency, nocturnal frontal lobe epilepsy, Joubert syndrome, Melnick-Fraser syndrome, Osteopetrosis with renal tubular acidosis, carbonic anhydrase II variant, Achromatopsia, Hereditary cancer-predisposing syndrome, Microcephaly, normal intelligence and immunodeficiency, Microcephaly, normal intelligence and immunodeficiency, Joubert syndrome, Meckel syndrome type 3, Nephronophthisis, Meckel-Gruber syndrome, coach syndrome, Pyruvate dehydrogenase phosphatase deficiency, Carcinoma of colon, Leigh syndrome, multiple synostoses syndrome, Microphthalmia, isolated, Klippel-Feil syndrome, autosomal dominant, Leber congenital amaurosis, Klippel-Feil syndrome, autosomal dominant, Anauxetic dysplasia, Cohen syndrome, Cohen syndrome, Abnormality of the eye, Ciliary dyskinesia, primary, 28, Epilepsy, nocturnal frontal lobe, Corneal dystrophy, corneal dystrophy, posterior polymorphous, RRM2B-related mitochondrial disease, Mitochondrial DNA depletion syndrome, encephalomyopathic form, with renal tubulopathy, RRM2B-related mitochondrial disease, Nail disorder, nonsyndromic congenital, Nail disease, Dihydropyrimidinase deficiency, Tetraamelia syndrome, Trichorhinophalangeal dysplasia type I, Multiple congenital exostosis, Dandy-Walker like malformation with atrioventricular septal defect, Benign familial neonatal seizures, Ciliary dyskinesia, primary, Iodotyrosyl coupling defect, Mental retardation, autosomal recessive, Deficiency of steroid 11-beta-monooxygenase, Corticosterone methyloxidase type 1 deficiency, Hyperlipoproteinemia, type ID, Amelogenesis imperfecta, hypocalcification type, 5-Oxoprolinase deficiency, Mitochondrial complex III deficiency, nuclear type 6, Brown-Vialetto-Van Laere syndrome, Hereditary acrodermatitis enteropathica, Rothmund-Thomson syndrome, Baller-Gerold syndrome, Hyperimmunoglobulin E recurrent infection syndrome, autosomal recessive, Nicolaides-Baraitser syndrome, Cerebellar ataxia, mental retardation, and dysequilibrium syndrome, Retinal cone dystrophy, Familial erythrocytosis, Chronic myelogenous leukemia, Polycythemia vera, Budd-Chiari syndrome, Myelofibrosis, Budd-Chiari syndrome, susceptibility to, somatic, Acute myeloid leukemia, Thrombocythemia, Myeloproliferative disorder, Subacute lymphoid leukemia, Non-ketotic hyperglycinemia, Hydrocephalus, Melanoma-pancreatic cancer syndrome, Hereditary cutaneous melanoma, Hereditary cancer-predisposing syndrome, Cutaneous malignant melanoma, Hereditary cutaneous melanoma, Hereditary cancer-predisposing syndrome, Hereditary cutaneous melanoma, Melanoma-pancreatic cancer syndrome, Hereditary cutaneous melanoma, Hereditary cancer-predisposing syndrome, neurodevelopmental disorder with progressive microcephaly, spasticity, and brain anomalies, Bardet-Biedl syndrome, Glaucoma, primary congenital, Singleton-Merten syndrome, Ciliary dyskinesia, Distal spinal muscular atrophy, autosomal recessive, Deficiency of UDPglucose-hexose-1-phosphate uridylyltransferase, Deficiency of UDPglucose-hexose-1-phosphate uridylyltransferase, Galactosemia, Inclusion body myopathy with early-onset paget disease and frontotemporal dementia, Fanconi anemia, complementation group G, Metaphyseal chondrodysplasia, McKusick type, Acromesomelic dysplasia Maroteaux type, Inclusion body myopathy, Nonaka myopathy, Sialuria, GNE myopathy, Sialuria, Inclusion body myopathy, Nonaka myopathy, Sialuria, Primary hyperoxaluria, type II, Pontocerebellar hypoplasia, type 1b, Friedreich's ataxia, Progressive familial intrahepatic cholestasis, Hypomagnesemia, intestinal, Cone-rod dystrophy and hearing loss, Obesity, hyperphagia, and developmental delay, AGTPBP1-related condition, Type B brachydactyly, Fructose-biphosphatase deficiency, Fanconi anemia, Fanconi anemia, complementation group C, Hereditary cancer-predisposing syndrome, Gorlin syndrome, Gorlin syndrome, Hereditary cancer-predisposing syndrome, Xeroderma pigmentosum, type 1, Spondyloepimetaphyseal dysplasia Genevieve type, Early infantile epileptic encephalopathy 59, Loeys-Dietz syndrome, Thoracic aortic aneurysm and aortic dissection, Loeys-Dietz syndrome, Congenital disorder of glycosylation type 1, Hereditary fructosuria, Familial hypoalphalipoproteinemia, Tangier disease, Limb-girdle muscular dystrophy-dystroglycanopathy, type C4, Congenital muscular dystrophy-dystroglycanopathy with brain and eye anomalies, type A4, Primary autosomal recessive microcephaly, Meretoja syndrome, adrenal insufficiency, NR5A1-related, 46,XY sex reversal, type 3, Nail-patella syndrome, Early infantile epileptic encephalopathy 4, Epileptic encephalopathy, Primary pulmonary hypertension, Osler hemorrhagic telangiectasia syndrome, Coenzyme Q10 deficiency, primary, Ichthyosis prematurity syndrome, Congenital disorder of glycosylation type 1M, Citrullinemia type I, Citrullinemia type I, Citrullinemia, mild, Neuropathy, hereditary sensory and autonomic, type VIII, short stature, hearing loss, retinis pigmentosa, and distinctive facies, Cortical malformations, occipital, Limb-girdle muscular dystrophy-dystroglycanopathy, type C1, Congenital muscular dystrophy-dystroglycanopathy with mental retardation, type B1, Walker-Warburg congenital muscular dystrophy, Spinocerebellar ataxia autosomal recessive, Tuberous sclerosis syndrome, Tuberous sclerosis, Lymphangiomyomatosis, Congenital nonprogressive myopathy with Moebius and Robin sequences, Dopamine beta hydroxylase deficiency, Ehlers-Danlos syndrome, type 2, Ehlers-Danlos syndrome, classic type, Early infantile epileptic encephalopathy, Epilepsy, nocturnal frontal lobe, Joubert syndrome, Adams-Oliver syndrome, Aortic valve disorder, Adams-Oliver syndrome, Congenital generalized lipodystrophy type 1, Neurodevelopmental disorder with or without hyperkinetic movements and seizures, autosomal dominant, Autosomal recessive hypophosphatemic bone disease, Chromosome 9q deletion syndrome, Neoplasm of stomach, Prostate cancer, somatic, Refsum disease, adult, Severe combined immunodeficiency, athabascan-type, Renal adysplasia, Megaloblastic anemia due to inborn errors of metabolism, Primary ciliary dyskinesia, Kartagener syndrome, Desanto-shinawi syndrome, Neural tube defect, Familial medullary thyroid carcinoma, Multiple endocrine neoplasia, type 2, MEN2A and FMTC, Multiple endocrine neoplasia, type 2, MEN2A and Unclassified, MEN2A and FMTC, Multiple endocrine neoplasia, type 2, MEN2A and FMTC, Hereditary cancer-predisposing syndrome, Multiple endocrine neoplasia, type 2b, Familial medullary thyroid carcinoma, Multiple endocrine neoplasia, type 2a, Multiple endocrine neoplasia, type 2, MEN2A and FMTC, FMTC and Unclassified, Multiple endocrine neoplasia, type 2a, Hereditary cancer-predisposing syndrome, Pheochromocytoma, Familial medullary thyroid carcinoma, Multiple endocrine neoplasia, type 2a, Multiple endocrine neoplasia, type 2, MEN2A and FMTC, Medullary thyroid carcinoma, Multiple endocrine neoplasia, type 2a, Multiple endocrine neoplasia, type 2, MEN2 phenotype: Unknown, Hereditary cancer-predisposing syndrome, Multiple endocrine neoplasia, type 2b, Multiple endocrine neoplasia, type 2a, MEN2 phenotype: Unclassified, Multiple endocrine neoplasia, type 2, MEN2A and FMTC, Hereditary cancer-predisposing syndrome, Familial medullary thyroid carcinoma, Multiple endocrine neoplasia, type 2a, MEN2A and FMTC, Hereditary cancer-predisposing syndrome, Multiple endocrine neoplasia, Telangiectasia, hereditary hemorrhagic, type 5, Cockayne syndrome B, Premature ovarian failure, Familial infantile myasthenia, Charcot-Marie-Tooth disease, demyelinating, type 1d, Congenital hypomyelinating neuropathy, Neuropathy, congenital hypomyelinating, autosomal dominant, Shprintzen-Goldberg syndrome, Goldberg-Shprintzen megacolon syndrome, Shprintzen-Goldberg syndrome, Diarrhea, malabsorptive, congenital, Aplastic anemia, Hemophagocytic lymphohistiocytosis, familial, nephrotic syndrome, Hyperphenylalaninemia, BH4-deficient, D, Histiocytosis-lymphadenopathy plus syndrome, Usher syndrome, type 1D, pituitary adenoma, multiple types, Usher syndrome, type 1D, Usher syndrome, type 1D, Gaucher disease, atypical, due to saposin C deficiency, Krabbe disease atypical due to Saposin A deficiency, Combined saposin deficiency, Sphingolipid activator protein deficiency, Gaucher disease, atypical, due to saposin C deficiency, Spondyloepiphyseal dysplasia with congenital joint dislocations, Dilated cardiomyopathy 1W, Familial hypertrophic cardiomyopathy, Cardiovascular phenotype, Hypermethioninemia due to adenosine kinase deficiency, Genitopatellar syndrome, Young Simpson syndrome, Hypomyelinating leukodystrophy, Idiopathic fibrosing alveolitis, chronic form, Hepatic methionine adenosyltransferase deficiency, Hereditary cancer-predisposing syndrome, Juvenile polyposis syndrome, Juvenile polyposis syndrome, Hereditary cancer-predisposing syndrome, Hyperinsulinism-hyperammonemia syndrome, Spondyloepimetaphyseal dysplasia, pakistani type, hyperekplexia, Cowden syndrome, PTEN hamartoma tumor syndrome, Hereditary cancer-predisposing syndrome, Hereditary cancer-predisposing syndrome, Neoplasm of the breast, PTEN hamartoma tumor syndrome, Malignant melanoma of skin, Squamous cell carcinoma of the head and neck, Small cell lung cancer, Squamous cell lung carcinoma, Renal cell carcinoma, papillary, Neoplasm of the breast, Glioblastoma, Hereditary cancer-predisposing syndrome, Colorectal Neoplasms, Uterine cervical neoplasms, Adenocarcinoma of stomach, Malignant neoplasm of body of uterus, Adenocarcinoma of prostate, Uterine Carcinosarcoma, PTEN hamartoma tumor syndrome, Cowden syndrome, Hereditary cancer-predisposing syndrome, Hereditary cancer-predisposing syndrome, Lhermitte-Duclos disease, Neoplasm of the breast, Colorectal Neoplasms, Hereditary cancer-predisposing syndrome, Macrocephaly/autism syndrome, Hereditary cancer-predisposing syndrome, PTEN hamartoma tumor syndrome, Cutaneous melanoma, Hereditary cancer-predisposing syndrome, PTEN hamartoma tumor syndrome, Hereditary cancer-predisposing syndrome, Autoimmune lymphoproliferative syndrome, type 1a, Lysosomal acid lipase deficiency, Microcephaly with or without chorioretinopathy, lymphedema, or mental retardation, Hydranencephaly with renal aplasia-dysplasia, Spastic paraplegia, Cutis laxa, autosomal dominant, Primary hyperoxaluria, type III, Spastic tetraparesis, Hermansky-Pudlak syndrome, Dubin-Johnson syndrome, Renal coloboma syndrome, Autosomal dominant progressive external ophthalmoplegia with mitochondrial DNA deletions, Mitochondrial diseases, Autosomal dominant progressive external ophthalmoplegia with mitochondrial DNA deletions, Mitochondrial diseases, Kallmann syndrome, Combined partial 17-alpha-hydroxylase/17,20-lyase deficiency, Complete combined 17-alpha-hydroxylase/17,20-lyase deficiency, Cerebroretinal microangiopathy with calcifications and cysts, Adult junctional epidermolysis bullosa, Epidermolysis bullosa, junctional, spermatogenic failure, Primary dilated cardiomyopathy, Dilated cardiomyopathy, Microphthalmia, syndromic, Myofibrillar myopathy, BAG3-related, Myofibrillar myopathy, BAG3-related, Dilated cardiomyopathy, Jackson-Weiss syndrome, Craniosynostosis, nonsyndromic unicoronal, Pfeiffer syndrome, Craniofacial-skeletal-dermatologic dysplasia, FGFR2 related craniosynostosis, Pfeiffer syndrome, FGFR2 related craniosynostosis, Cerebral arteriopathy, autosomal dominant, with subcortical infarcts and leukoencephalopathy, type 2, Ornithine aminotransferase deficiency, Congenital erythropoietic porphyria, Muscular hypotonia, Muscular hypotonia, Intellectual disability (severe), Hypotonia, ataxia, and delayed development syndrome, Global developmental delay, Expressive language delay, Intellectual disability, Ataxia, Muscular hypotonia, Hypotonia, ataxia, and delayed development syndrome, Mitochondrial short-chain enoyl-coa hydratase deficiency, Noonan syndrome, Follicular thyroid carcinoma, Spermatocytic seminoma, somatic, Spermatocytic seminoma, Neoplasm of the breast, Costello syndrome, Myopathy, congenital, with excess of muscle spindles, Liver cancer, Chronic lymphocytic leukemia, Malignant melanoma of skin, Multiple myeloma, Squamous cell carcinoma of the head and neck, Costello syndrome, Lung adenocarcinoma, Squamous cell lung carcinoma, Acute myeloid leukemia, Neoplasm of the breast, Hepatocellular carcinoma, Pancreatic adenocarcinoma, Squamous cell carcinoma of the skin, Transitional cell carcinoma of the bladder, Colorectal Neoplasms, Uterine cervical neoplasms, Thymoma, Adenocarcinoma of stomach, Malignant neoplasm of body of uterus, Liver cancer, Chronic lymphocytic leukemia, Malignant melanoma of skin, Multiple myeloma, Squamous cell carcinoma of the head and neck, Costello syndrome, Lung adenocarcinoma, Squamous cell lung carcinoma, Acute myeloid leukemia, Rasopathy, Neoplasm of the breast, Hepatocellular carcinoma, Pancreatic adenocarcinoma, Squamous cell carcinoma of the skin, Transitional cell carcinoma of the bladder, Neoplasm, Colorectal Neoplasms, Uterine cervical neoplasms, Neoplasm of the thyroid gland, Adenocarcinoma of stomach, Malignant neoplasm of body of uterus, Malignant tumor of urinary bladder, Costello syndrome, Epidermal nevus, Myopathy, congenital, with excess of muscle spindles, Cutaneous melanoma, Neoplasm of the thyroid gland, Liver cancer, Malignant melanoma of skin, Multiple myeloma, Squamous cell carcinoma of the head and neck, Costello syndrome, Epidermal nevus, Lung adenocarcinoma, Acute myeloid leukemia, Myelodysplastic syndrome, Nevus sebaceous, Nevus sebaceous, somatic, Rasopathy, Neoplasm of the breast, Glioblastoma, Bladder carcinoma, Hepatocellular carcinoma, Pancreatic adenocarcinoma, Squamous cell carcinoma of the skin, Transitional cell carcinoma of the bladder, Carcinoma of esophagus, Colorectal Neoplasms, Uterine cervical neoplasms, Neoplasm of the thyroid gland, Papillary renal cell carcinoma, sporadic, Adenoid cystic carcinoma, Nasopharyngeal Neoplasms, Adenocarcinoma of stomach, Ovarian Serous Cystadenocarcinoma, Malignant neoplasm of body of uterus, Adenocarcinoma of prostate, Uterine Carcinosarcoma, Early myoclonic encephalopathy, Neutral lipid storage disease with myopathy, Ceroid lipofuscinosis neuronal, Growth restriction, severe, with distinctive facies, Hyperproinsulinemia, Permanent neonatal diabetes mellitus, Hyperproinsulinemia, Segawa syndrome, autosomal recessive, Dystonia, Segawa syndrome, autosomal recessive, Jervell and Lange-Nielsen syndrome, Long QT syndrome, Cardiovascular phenotype, Congenital long QT syndrome, Long QT syndrome, Congenital long QT syndrome, Long QT syndrome, Long QT syndrome 1/2, digenic, Long QT syndrome, Congenital long QT syndrome, Cardiovascular phenotype, Long QT syndrome, Congenital long QT syndrome, Long QT syndrome, Cardiovascular phenotype, Beckwith-Wiedemann syndrome, Intrauterine growth retardation, metaphyseal dysplasia, adrenal hypoplasia congenita, and genital anomalies, Intrauterine growth retardation, metaphyseal dysplasia, adrenal hypoplasia congenita, and genital anomalies, Russell-Silver syndrome, Beckwith-Wiedemann syndrome, Myopathy with tubular aggregates, hemoglobin Ohio, erythrocytosis, hemoglobin TY gard, erythrocytosis, Beta-thalassemia, dominant inclusion body type, Hemoglobinopathy, Beta-plus-thalassemia, Beta thalassemia intermedia, hemoglobin Ypsilanti, erythrocytosis, beta\hat{ }0\hat{ } Thalassemia, Heinz body anemia, Beta-plus-thalassemia, Beta thalassemia major, beta\hat{ }0\hat{ } Thalassemia, beta Thalassemia, Beta-plus-thalassemia, Hemoglobin Knossos, Beta-knossos-thalassemia, beta Thalassemia, Hemoglobin Palmerston north, erythrocytosis, Hb niigata, Beta-plus-thalassemia, beta Thalassemia, Beta thalassemia intermedia, beta Thalassemia, delta Thalassemia, hemoglobin A(2) Yialousa, Fetal hemoglobin quantitative trait locus 1, Sphingomyelin/cholesterol lipidosis, Niemann-Pick disease, type B, Niemann-Pick disease, type A, Niemann-pick disease, intermediate, protracted neurovisceral, Sphingomyelin/cholesterol lipidosis, Niemann-Pick disease, type B, Niemann-Pick disease, type A, Niemann-Pick disease, type B, Niemann-Pick disease, type A, Sphingomyelin/cholesterol lipidosis, Ceroid lipofuscinosis neuronal, Neuronal ceroid lipofuscinosis, Van Maldergem syndrome, Permanent neonatal diabetes mellitus, Permanent neonatal diabetes mellitus, Diabetes mellitus, permanent neonatal, with neurologic features, Islet cell hyperplasia, Permanent neonatal diabetes mellitus, Persistent hyperinsulinemic hypoglycemia of infancy, Permanent neonatal diabetes mellitus, Hyperekplexia, Gnathodiaphyseal dysplasia, Limb-girdle muscular dystrophy, type 2L, Gnathodiaphyseal dysplasia, Limb-girdle muscular dystrophy, type 2L, Miyoshi muscular dystrophy, ANO5-Related Disorders, Limb-girdle muscular dystrophy, type 2L, Elevated serum creatine phosphokinase, Myopathy, Distal muscle weakness, Fatty replacement of skeletal muscle, Limb-girdle muscular dystrophy, type 2L, Follicle-stimulating hormone deficiency, isolated, Aniridia, Irido-corneo-trabecular dysgenesis, Foveal hypoplasia with cataract, Irido-corneo-trabecular dysgenesis, Anophthalmia-microphthalmia, Aniridia, Irido-corneo-trabecular dysgenesis, Wilms tumor, Combined cellular and humoral immune defects with granulomas, Severe combined immunodeficiency, B cell-negative, Histiocytic medullary reticulosis, Severe immunodeficiency, autosomal recessive, T-cell negative, B-cell negative, NK cell-positive, Combined cellular and humoral immune defects with granulomas, Multiple exostoses type 2, Parietal foramina, Congenital disorder of glycosylation type 2C, Thrombophilia, Hereditary factor II deficiency disease, Xeroderma pigmentosum, group E, Left ventricular noncompaction, Hypertrophic cardiomyopathy, Primary familial hypertrophic cardiomyopathy, Hypertrophic cardiomyopathy, Cardiovascular phenotype, Primary familial hypertrophic cardiomyopathy, Cardiovascular phenotype, Familial hypertrophic cardiomyopathy, Primary familial hypertrophic cardiomyopathy, Hypertrophic, Primary familial hypertrophic cardiomyopathy, Cardiovascular phenotype, Familial hypertrophic cardiomyopathy, Familial hypertrophic cardiomyopathy, Primary familial hypertrophic cardiomyopathy, Hypertrophic cardiomyopathy, Cardiovascular phenotype, Primary familial hypertrophic cardiomyopathy, Hypertrophic cardiomyopathy, Primary familial hypertrophic cardiomyopathy, Primary familial hypertrophic cardiomyopathy, Hypertrophic cardiomyopathy, Hypertrophic cardiomyopathy, Primary familial hypertrophic cardiomyopathy, Cardiovascular phenotype, Familial hypertrophic cardiomyopathy, Cardiovascular phenotype, Primary familial hypertrophic cardiomyopathy, Familial hypertrophic cardiomyopathy, Hypertrophic cardiomyopathy, Myasthenic syndrome, congenital, associated with acetylcholine receptor deficiency, Pena-Shokeir syndrome type I, Myasthenic syndrome, congenital, associated with acetylcholine receptor deficiency, Congenital myasthenic syndrome, Myopathy, Myasthenic syndrome, congenital, associated with acetylcholine receptor deficiency, Congenital Myasthenic Syndrome, Recessive, Myasthenic syndrome, congenital, associated with acetylcholine receptor deficiency, Hereditary angioedema type 1, Hereditary C1 esterase inhibitor deficiency—dysfunctional factor, Poikiloderma, hereditary fibrosing, with tendon contractures, myopathy, and pulmonary fibrosis, Gracile bone dysplasia, Joubert syndrome, Joubert syndrome, Meckel syndrome type, Retinal dystrophy, polycystic kidney disease with polycystic liver disease, Congenital generalized lipodystrophy type 2, Charcot-Marie-Tooth disease, type 2, Encephalopathy, progressive, with or without lipodystrophy, Familial renal hypouricemia, Platelet-type bleeding disorder, Glycogen storage disease, type V, Hereditary cancer-predisposing syndrome, Multiple endocrine neoplasia, type 1, Hereditary cancer-predisposing syndrome, Hereditary cancer-predisposing syndrome, Multiple endocrine neoplasia, type 1, Multiple endocrine neoplasia, type 1, Hereditary cancer-predisposing syndrome, Coffin-Siris syndrome, Calfan syndrome, Verloes Bourguignon syndrome, Bardet-Biedl syndrome, Bardet-Biedl syndrome, Spinocerebellar ataxia, autosomal recessive, Pyruvate carboxylase deficiency, Cold-induced sweating syndrome, Crisponi/Cold-induced sweating syndrome, Somatotroph adenoma, Pituitary adenoma predisposition, Mitochondrial complex I deficiency, Osteopetrosis autosomal recessive, Severe congenital neutropenia autosomal dominant, congenital neutropenia, High bone mass, Osteoporosis with pseudoglioma, Epilepsy, familial temporal lobe, Carnitine palmitoyltransferase I deficiency, Charcot-Marie-Tooth disease, Charcot-Marie-Tooth disease, axonal, type 2S, IGHMBP2-related condition, Spinal muscular atrophy, distal, autosomal recessive, Charcot-Marie-Tooth disease, axonal, type 2S, Werdnig-Hoffmann disease, Charcot-Marie-Tooth disease, axonal, type 2S, Deafness with labyrinthine aplasia microtia and microdontia (LAMM), Smith-Lemli-Opitz syndrome, Cerebral folate deficiency, Opsismodysplasia, 3-methylglutaconic aciduria with cataracts, neurologic involvement, and neutropenia, Joubert syndrome, Vitreoretinopathy, neovascular inflammatory, Usher syndrome, type 1, Usher syndrome, type 1, Usher syndrome, type 1B, Usher syndrome, type 1, MYO7A-Related Disorders, polycystic liver disease with or without kidney cysts, Tremor, hereditary essential, Mitochondrial complex I deficiency, Mitochondrial diseases, Tyrosinase-negative oculocutaneous albinism, Tyrosinase-negative oculocutaneous albinism, Oculocutaneous albinism type 1B, Albinism, ocular, with sensorineural deafness, Skin/hair/eye pigmentation, variation in, Oculocutaneous albinism, Hereditary cancer-predisposing syndrome, Ataxia-telangiectasia-like disorder, Charcot-Marie-Tooth disease, type 4B1, Focal segmental glomerulosclerosis, Coloboma, ocular, with or without hearing impairment, cleft lip/palate, and/or mental retardation, Metaphyseal chondrodysplasia, Spahr type, Short-rib polydactyly syndrome type III, Jeune thoracic dystrophy, Short-rib thoracic dysplasia with or without polydactyly, Short-rib polydactyly syndrome type I, Short-rib polydactyly syndrome type III, Deficiency of acetyl-CoA acetyltransferase, Hereditary cancer-predisposing syndrome, Ataxia-telangiectasia syndrome, Ataxia-telangiectasia syndrome, Ataxia-telangiectasia variant, Pyruvate dehydrogenase E2 deficiency, Pheochromocytoma, Paragangliomas, Hereditary cancer-predisposing syndrome, Paragangliomas, Hereditary Paraganglioma-Pheochromocytoma Syndromes, Paraganglioma and gastric stromal sarcoma, Pheochromocytoma, Paragangliomas, Hereditary Paraganglioma-Pheochromocytoma Syndromes, Cowden syndrome, Paraganglioma and gastric stromal sarcoma, Pheochromocytoma, Mitochondrial complex II deficiency, Paragangliomas, Hereditary Paraganglioma-Pheochromocytoma Syndromes, Cowden syndrome 3, Apolipoprotein A-IV polymorphism, APOA4*1/APOA4*2, Hyperalphalipoproteinemia, Coronary heart disease, Apolipoprotein A-I (Baltimore), Immunodeficiency, Kabuki syndrome, Wiedemann-Steiner syndrome, Short stature, rhizomelic, with microcephaly, micrognathia, and developmental delay, Glucose-6-phosphate transport defect, Acute intermittent porphyria, Congenital myasthenic syndrome, Noonan syndrome-like disorder with or without juvenile myelomonocytic leukemia, Microphthalmia, isolated, Gaze palsy, familial horizontal, with progressive scoliosis, Megalencephalic leukoencephalopathy with subcortical cysts 2a, Deficiency of isobutyryl-CoA dehydrogenase, Cone dystrophy, Retinal cone dystrophy, Megalencephaly-polymicrogyria-polydactyly-hydrocephalus syndrome, Tumoral calcinosis, familial, hyperphosphatemic, Episodic ataxia type 1, Myokymia, Atrial fibrillation, familial, von Willebrand disease type 3, von Willebrand disease type 2N, von Willebrand disease type 2N, TNF receptor-associated periodic fever syndrome (TRAPS), Sifrim-Hitz-Weiss syndrome, Triosephosphate isomerase deficiency, Ehlers-Danlos syndrome, type 8, Immunodeficiency with hyper IgM type 2, Aortic aneurysm, familial thoracic, Acute myeloid leukemia, Diarrhea, Brachydactyly with hypertension, Hypoglycemia with deficiency of glycogen synthetase in the liver, Lamb-shaffer syndrome, Non-small cell lung cancer, Colorectal Neoplasms, Neoplasm of the thyroid gland, Non-small cell lung cancer, Rasopathy, on-small cell lung cancer, Colorectal Neoplasms, Neoplasm of the thyroid gland, cetuximab response—Dosage, panitumumab response—Dosage, Non-small cell lung cancer, RAS-associated autoimmune leukoproliferative disorder, Colorectal Neoplasms, Cerebral arteriovenous malformation, Juvenile myelomonocytic leukemia, Carcinoma of pancreas, Non-small cell lung cancer, Acute myeloid leukemia, Nevus sebaceous, Nevus sebaceous, somatic, Ovarian Neoplasms, Colorectal Neoplasms, Neoplasm of the thyroid gland, Endometrial carcinoma, Lung cancer, Lung adenocarcinoma, Non-small cell lung cancer, Ovarian Neoplasms, Colorectal Neoplasms, Neoplasm of the thyroid gland, Charcot-Marie-Tooth disease, type 4H, Optic atrophy, Encephalopathy due to defective mitochondrial and peroxisomal fission, Arrhythmogenic right ventricular cardiomyopathy, Arrhythmogenic right ventricular cardiomyopathy, type 9, Arrhythmogenic right ventricular dysplasia/cardiomyopathy, Cardiovascular phenotype, Parkinson disease, late-onset, Parkinson disease, autosomal dominant, IRAK4 deficiency, Vitamin D-dependent rickets, type 2, Spondyloperipheral dysplasia, Short ribs, Absent vertebral body mineralization, Spondylometaphyseal dysplasia, Stickler syndrome type 1, Stickler syndrome, type I, nonsyndromic ocular, Achondrogenesis, type II, Stickler syndrome type 1, Spondylometaphyseal dysplasia, Spondylometaphyseal dysplasia, Stickler syndrome, type I, nonsyndromic ocular, Glycogen storage disease, type VII, Glycogen storage disease, type VII, Osteogenesis imperfecta, type xv, Osteogenesis imperfecta, type xv, Osteogenesis imperfecta, type xv, Kabuki syndrome, Smith-Magenis Syndrome-like, Lissencephaly, Diabetes insipidus, nephrogenic, autosomal recessive, Diffuse palmoplantar keratoderma, Bothnian type, Hypochromic microcytic anemia with iron overload, Early infantile epileptic encephalopathy, Hereditary hemorrhagic telangiectasia type 2, Pulmonary arterial hypertension related to hereditary hemorrhagic telangiectasia, Primary pulmonary hypertension, Beaded hair, Pachyonychia congenita, Epidermolysis bullosa simplex, Dowling-Meara type, with severe palmoplantar keratoderma, Epidermolysis bullosa simplex, Cockayne-Touraine type, Epidermolysis bullosa simplex, Koebner type, Dowling-Degos disease, Ichthyosis bullosa of Siemens, Bullous ichthyosiform erythroderma, Cirrhosis, cryptogenic, Cirrhosis, noncryptogenic, susceptibility to, Glucocorticoid deficiency with achalasia, Ectodermal dysplasia, hair/nail type, Pigmentary retinal dystrophy, Fundus albipunctatus, autosomal recessive, Sulfite oxidase deficiency, isolated, Immunodeficiency, Congenital cataract, axonal, type 2u, Nephrotic syndrome, type 11, Bardet-Biedl syndrome, Myopathy, centronuclear, Joubert syndrome, Leber congenital amaurosis, Meckel syndrome type 4, Senior-Loken syndrome, Bardet-Biedl syndrome, Joubert syndrome, Leber congenital amaurosis, Meckel-Gruber syndrome, Meckel syndrome type 4, Senior-Loken syndrome, Joubert syndrome, Bardet-Biedl syndrome, Nephronophthisis, Meckel-Gruber syndrome, Nephronophthisis, Leber congenital amaurosis, Meckel syndrome type 4, Senior-Loken syndrome, Meckel-Gruber syndrome, Nephronophthisis, CEP290-Related Disorders, Leber congenital amaurosis, Meckel syndrome type 4, Senior-Loken syndrome, Leber congenital amaurosis, Meckel syndrome type 4, Senior-Loken syndrome, Cone-rod dystrophy, Cornea plana, Nephronophthisis, I cell disease, Pseudo-Hurler polydystrophy, Phenylketonuria, Hyperphenylalaninemia, non-pku, Congenital central hypoventilation, Hypomyelinating leukodystrophy, with or without oligodontia and/or hypogonadotropic hypogonadism, Methylmalonic aciduria cblB type, Methylmalonic academia, Spondylometaphyseal dysplasia, Kozlowski type, Skeletal dysplasia, Charcot-Marie-Tooth disease type 2C, Skeletal dysplasia, Neuromuscular Diseases, Digital arthropathy-brachydactyly, familial, Metatrophic dysplasia, Spondylometaphyseal dysplasia, Distal spinal muscular atrophy, congenital nonprogressive, Scapuloperoneal spinal muscular atrophy, Charcot-Marie-Tooth disease type 2C, Skeletal dysplasia, Neuromuscular Diseases, Charcot-Marie-Tooth, Type 2, Brachyolmia, Metatrophic dysplasia, Skeletal dysplasia, Neuromuscular Diseases, Darier disease, acral hemorrhagic type, Darier disease, segmental, Familial hypertrophic cardiomyopathy, Primary familial hypertrophic cardiomyopathy, Familial hypertrophic cardiomyopathy, Death in infancy, Ventricular extrasystoles, Cardiovascular phenotype, Noonan syndrome, Noonan syndrome, Rasopathy, Juvenile myelomonocytic leukemia, Noonan syndrome, Leopard syndrome, Rasopathy, Metachondromatosis, Noonan syndrome with multiple lentigines, Noonan syndrome 1, LEOPARD syndrome, Scoliosis, Rasopathy, Abnormal facial shape, Cafe-au-lait spot, Specific learning disability, Intellectual disability, mild, Aortic valve disease, Holt-Oram syndrome, Mental retardation and distinctive facial features with or without cardiac defects, Charcot-Marie-Tooth disease, type 2L, Microcephaly, primary, autosomal recessive, Deficiency of butyryl-CoA dehydrogenase, Maturity-onset diabetes of the young, type 3, Immune dysfunction with T-cell inactivation due to calcium entry defect, Leukoencephalopathy with vanishing white matter, Joubert syndrome, Cutis laxa with osteodystrophy, Myopathy, lactic acidosis, and sideroblastic anemia, Knuckle pads, deafness and leukonychia syndrome, Keratitis-ichthyosis-deafness syndrome, autosomal dominant, Mutilating keratoderma, Hystrix-like ichthyosis with deafness, Keratitis-ichthyosis-deafness syndrome, autosomal dominant, Keratoderma palmoplantar deafness, Knuckle pads, deafness and leukonychia syndrome, Deafness, X-linked, Hearing impairment, Keratoderma palmoplantar deafness, Cardiomyopathy, Left ventricular noncompaction, Cardiomyopathy, Infantile muscular hypotonia, Combined oxidative phosphorylation deficiency, Pancreatic agenesis, congenital, Diabetes mellitus type 2, Acute lymphoid leukemia, Acute myeloid leukemia, Breast-ovarian cancer, familial, Hereditary breast and ovarian cancer syndrome, Breast-ovarian cancer, familial, Hereditary breast and ovarian cancer syndrome, Hereditary cancer-predisposing syndrome, Hereditary breast and ovarian cancer syndrome, Familial cancer of breast, Breast-ovarian cancer, familial, Hereditary breast and ovarian cancer syndrome, Hereditary cancer-predisposing syndrome, Familial cancer of breast, Breast-ovarian cancer, familial, Fanconi anemia, complementation group D1, Medulloblastoma, Wilms tumor, Malignant tumor of prostate, Tracheoesophageal fistula, Pancreatic cancer, Glioma susceptibility, Hereditary breast and ovarian cancer syndrome, Hereditary cancer-predisposing syndrome, Familial cancer of breast, Breast-ovarian cancer, familial, Hereditary breast and ovarian cancer syndrome, Neoplasm of the breast, Hereditary cancer-predisposing syndrome, Familial cancer of breast, Breast-ovarian cancer, familial, Fanconi anemia, complementation group D1, Medulloblastoma, Wilms tumor, Malignant tumor of prostate, Tracheoesophageal fistula, Pancreatic cancer, Glioma susceptibility, Hereditary breast and ovarian cancer syndrome, Hereditary cancer-predisposing syndrome, BRCA2-Related Disorders, Breast-ovarian cancer, familial, Fanconi anemia, complementation group D1, Fanconi anemia, Hereditary breast and ovarian cancer syndrome, Hereditary cancer-predisposing syndrome, Primary pulmonary hypertension, Congenital disorder of glycosylation type 2L, Hyperornithinemia-hyperammonemia-homocitrullinuria syndrome, Retinoblastoma, Retinoblastoma, Neoplasm, Small cell lung cancer, Neoplasm, Retinitis pigmentosa, Retinal dystrophy with or without extraocular anomalies, Retinitis pigmentosa, Retinal dystrophy with extraocular anomalies, Aicardi Goutieres syndrome, Wilson disease, Ceroid lipofuscinosis neuronal, Hirschsprung disease, Waardenburg syndrome type 4A, Deafness and myopia, Catel Manzke syndrome, Propionyl-CoA carboxylase deficiency, Hypotonia, infantile, with psychomotor retardation and characteristic facies, Congenital contractures of the limbs and face, hypotonia, and developmental delay, Xeroderma pigmentosum, group G, Xeroderma pigmentosum group g/Cockayne syndrome, Xeroderma pigmentosum, group G, Xeroderma pigmentosum, Schizencephaly, Angiopathy, hereditary, with nephropathy, aneurysms, and muscle cramps, Squamous cell carcinoma of the head and neck, Oguchi disease, Cone-rod dystrophy, Leber congenital amaurosis, Cone-Rod Dystrophy, Recessive, Autism, susceptibility to, Ocular coloboma, autosomal recessive, Lysinuric protein intolerance, Primary dilated cardiomyopathy, Wolff-Parkinson-White pattern, Dilated cardiomyopathy 1EE, Familial hypertrophic cardiomyopathy, Primary familial hypertrophic cardiomyopathy, Sudden cardiac death, Cardiovascular phenotype, Hypertrophic cardiomyopathy, Primary familial hypertrophic cardiomyopathy, Cardiovascular phenotype, Familial hypertrophic cardiomyopathy, Primary familial hypertrophic cardiomyopathy, Cardiovascular phenotype, Familial hypertrophic cardiomyopathy, Familial cardiomyopathy, Hypertrophic cardiomyopathy, Cardiomyopathy, Hypertrophic cardiomyopathy, Dyskeratosis congenita, Dyskeratosis congenita autosomal dominant, Dyskeratosis congenita autosomal dominant, Dyskeratosis congenita, autosomal dominant, Revesz syndrome, Dyskeratosis congenita autosomal dominant, Dyskeratosis congenita, Dyskeratosis Congenita, Dominant, Autosomal recessive congenital ichthyosis, Rett syndrome, congenital variant, Mitochondrial complex I deficiency, Ectodermal dysplasia, anhidrotic, with T-cell immunodeficiency, autosomal dominant, Benign hereditary chorea, Choreoathetosis, hypothyroidism, and neonatal respiratory distress, Partial congenital absence of teeth, Ciliary dyskinesia, primary, Kartagener syndrome, L-2-hydroxyglutaric aciduria, Penetrating foot ulcers, Distal sensory impairment, Osteomyelitis leading to amputation due to slow healing fractures, Distal lower limb muscle weakness, Glycogen storage disease, type VI, Dystonia, Dopa-responsive type, Microphthalmia syndromic, Anophthalmia, combined immunodeficiency and megaloblastic anemia, Hereditary cancer-predisposing syndrome, congential disorder of glycosylation with defective fucosylation, Leber congenital amaurosis, Platelet-type bleeding disorder, Alzheimer disease, type 3, Alzheimer disease, type 3, Pick's disease, Alzheimer disease, type 3, Frontotemporal dementia, Pick's disease, Acne inversa, familial, Coenzyme Q10 deficiency, primary, Methylmalonate semialdehyde dehydrogenase deficiency, Niemann-Pick disease type C2, Niemann-Pick disease, type C, Leukoencephalopathy with vanishing white matter, Carcinoma of colon, Endometrial carcinoma, Hereditary nonpolyposis colorectal cancer type 7, Lynch syndrome, MLH3-Related Lynch Syndrome, Nevus comedonicus, Proliferative vasculopathy and hydranencephaly-hydrocephaly syndrome, Cone-rod dystrophy, Congenital muscular dystrophy-dystroglycanopathy with brain and eye anomalies, type A2, Congenital muscular dystrophy-dystroglycanopathy with mental retardation, type B2, Limb-girdle muscular dystrophy-dystroglycanopathy, type C2, Neuropathy, hereditary sensory, type IC, Hereditary sensory and autonomic neuropathy type IC, Thyroid adenoma, hyperfunctioning, somatic, Thyroid adenoma, hyperfunctioning, Hypothyroidism, congenital, nongoitrous, Hyperthyroidism, nonautoimmune, Thyroid adenoma, hyperfunctioning, somatic, Thyroid adenoma, hyperfunctioning, Galactosylceramide beta-galactosidase deficiency, Leber congenital amaurosis, Autosomal recessive cutis laxa type IA, TRIP11-related condition, Alpha-1-antitrypsin deficiency, Pineoblastoma, DICER1-related pleuropulmonary blastoma cancer predisposition syndrome, Hereditary cancer-predisposing syndrome, Gabriele-De Vries Syndrome, Spinal muscular atrophy, SMA, Spinal muscular atrophy, lower extremity predominant, autosomal dominant, Mental retardation, autosomal dominant, Mental retardation, autosomal dominant, Charcot-Marie-Tooth disease, dominant intermediate E, cerebellar-facial-dental syndrome, Cerebellofaciodental syndrome, Precocious puberty, central, Schaaf-yang syndrome, Angelman syndrome, Epileptic encephalopathy, early infantile, Tyrosinase-positive oculocutaneous albinism, Congenital stationary night blindness, type 1C, Andermann syndrome, Familial hypertrophic cardiomyopathy, Familial pulmonary capillary hemangiomatosis, Isovaleric acidemia, type I, Adams-Oliver syndrome, Limb-girdle muscular dystrophy, type 2A, Spherocytosis type 5, Peeling skin syndrome, Peeling skin syndrome, acral type, Microcephaly and chorioretinopathy, autosomal recessive, Hypoproteinemia, hypercatabolic, Arginine:glycine amidinotransferase deficiency, Bartter syndrome, type 1, antenatal, Marfan syndrome, Marfan lipodystrophy syndrome, Cardiovascular phenotype, Marfan syndrome, Thoracic aortic aneurysm and aortic dissection, Thoracic aortic Aneurysm and dissection (TAAD), Cardiovascular phenotype, Stiff skin syndrome, Marfan syndrome, Thoracic aortic aneurysm and aortic dissection, Thoracic aortic Aneurysm and dissection (TAAD), Marfan Syndrome/Loeys-Dietz Syndrome/Familial Thoracic Aortic Aneurysms and Dissections, Cardiovascular phenotype, Seckel syndrome, Aromatase deficiency, Lethal congenital contracture syndrome, Intellectual developmental disorder with cardiac arrhythmia, Primary ciliary dyskinesia, Craniosynostosis, Parkinson disease, age at onset, susceptibility to, Parkinson disease, Parkinson disease, autosomal recessive early-onset, Hyperchlorhidrosis, isolated, Nemaline myopathy, Congenital stationary night blindness, type 1D, Lung adenocarcinoma, Non-small cell lung cancer, Cutaneous melanoma, Cardio-facio-cutaneous syndrome, Cardiofaciocutaneous syndrome, Cardio-facio-cutaneous syndrome, Aortic valve disease, Thoracic aortic aneurysm and aortic dissection, Cardiovascular phenotype, Loeys-Dietz syndrome, Ceroid lipofuscinosis neuronal, Tay-Sachs disease, Bardet-Biedl syndrome, Sick sinus syndrome, autosomal dominant, Tyrosinemia type I, Tyrosinemia type I, Hypertyrosinemia, Osteochondritis dissecans, Autosomal dominant progressive external ophthalmoplegia with mitochondrial DNA deletions 1, Progressive sclerosing poliodystrophy, Progressive sclerosing poliodystrophy, Mitochondrial diseases, Camptocormia, Acrocallosal syndrome, Schinzel type, Spondylocostal dysostosis, Liver cancer, Acute myeloid leukemia, Neoplasm of brain, Hepatocellular carcinoma, Brainstem glioma, Colorectal Neoplasms, Multiple myeloma, Squamous cell carcinoma of the head and neck, Acute myeloid leukemia, Myelodysplastic syndrome, Colorectal Neoplasms, Bloom syndrome, Bloom syndrome, Hereditary cancer-predisposing syndrome, Arthrogryposis renal dysfunction cholestasis syndrome, Epileptic encephalopathy, childhood-onset, Congenital heart defects, multiple types, Weill-Marchesani-like syndrome, Autosomal recessive congenital ichthyosis, Microphthalmia, isolated, Osteosclerotic metaphyseal dysplasia, alpha Thalassemia, Hemoglobin Loire, Erythrocytosis, Hemoglobin Chesapeake, Erythrocytosis, Hemoglobin Legnano, Erythrocytosis, Spinocerebellar ataxia, autosomal recessive, Mucolipidosis III Gamma, You-Hoover-Fong syndrome, Renal dysplasia, retinal pigmentary dystrophy, cerebellar ataxia and skeletal dysplasia, Joubert syndrome with Jeune asphyxiating thoracic dystrophy, Renal dysplasia, retinal pigmentary dystrophy, cerebellar ataxia and skeletal dysplasia, Retinis pigmentosa, Leigh syndrome, Combined oxidative phosphorylation deficiency, Tuberous sclerosis, Tuberous sclerosis syndrome, Lymphangiomyomatosis, Tuberous sclerosis syndrome, Polycystic kidney disease, adult type, Digitorenocerebral syndrome, Early infantile epileptic encephalopathy, Myoclonic epilepsy, familial infantile, Digitorenocerebral syndrome, Progressive myoclonus epilepsy with ataxia, Familial Mediterranean fever, Rubinstein-Taybi syndrome, Nephronophthisis, Congenital disorder of glycosylation type 1K, Carbohydrate-deficient glycoprotein syndrome type I, Carbohydrate-deficient glycoprotein syndrome type I, Congenital disorder of glycosylation, Epilepsy, focal, with speech disorder and with or without mental retardation, Rolandic epilepsy, Bare lymphocyte syndrome type 2, complementation group A, Charcot-Marie-Tooth disease, type 1C, Fanconi anemia, complementation group Q, Dyskeratosis congenita, Dyskeratosis congenita, autosomal recessive, Lissencephaly, Aortic aneurysm, familial thoracic, Pseudoxanthoma elasticum, Pseudoxanthoma elasticum, Generalized arterial calcification of infancy, Familial juvenile gout, Uromodulin-associated kidney disease, Medullary cystic kidney disease, Bronchiectasis with or without elevated sweat chloride, Familial cancer of breast, Fanconi anemia, complementation group N, Tracheoesophageal fistula, Pancreatic cancer, Hereditary breast and ovarian cancer syndrome, Hereditary cancer-predisposing syndrome, Familial cancer of breast, Pancreatic cancer, Progressive sensorineural hearing impairment, IL21R immunodeficiency, Juvenile neuronal ceroid lipofuscinosis, Ceroid lipofuscinosis, neuronal, protracted, Brody myopathy, Spondyloepimetaphyseal dysplasia with multiple dislocations, Spondylocostal dysostosis, Bile acid synthesis defect, congenital, Generalized epilepsy with febrile seizures plus, type 9, Warfarin response, warfarin response—Dosage, Warfarin response, Familial renal glucosuria, Glycogen storage disease IXb, Behcet's syndrome, Cylindromatosis, familial, Townes-Brocks syndrome, Joubert syndrome, Hamamy syndrome, Multicentric osteolysis, nodulosis and arthropathy, Bardet-Biedl syndrome, Retinitis pigmentosa, Nephrotic syndrome, type 12, Familial hypokalemia-hypomagnesemia, Spondyloepimetaphyseal dysplasia, Faden-Alkuraya type, Polymicrogyria, bilateral frontoparietal, Lissencephaly, with microcephaly, Retinitis pigmentosa, Poikiloderma with neutropenia, Brachioskeletogenital syndrome, Mitochondrial DNA depletion syndrome, Lamellar cataract, Combined T and B cell immunodeficiency, Dyskeratosis congenita, autosomal dominant, Dyskeratosis congenita, autosomal recessive, Norum disease, Acanthosis nigricans, Skeletal dysplasia, Insulin resistance, Short stature, Self-injurious behavior, Abnormal facial shape, Brachydactyly, Renal hypoplasia, Abnormality of the dentition, Hepatic steatosis, Obesity, Lumbar hyperlordosis, Hyperlipidemia, Short metacarpal, Intellectual disability, severe, Short stature, brachydactyly, intellectual developmental disability, and seizures, Acanthosis nigricans, Skeletal dysplasia, Insulin resistance, Short stature, Self-injurious behavior, Abnormal facial shape, Brachydactyly, Renal hypoplasia, Abnormality of the dentition, Hepatic steatosis, Obesity, Lumbar hyperlordosis, Hyperlipidemia, Short metacarpal, Intellectual disability, severe, Hereditary diffuse gastric cancer, Hereditary cancer-predisposing syndrome, Ectropion inferior cleft lip and or palate, Breast cancer, lobular, Hereditary diffuse gastric cancer, Hereditary cancer-predisposing syndrome, Ectropion inferior cleft lip and or palate, Congenital disorder of glycosylation type 2J, Striatonigral degeneration, childhood-onset, Ciliary dyskinesia, primary, Kartagener syndrome, Tyrosinemia type 2, Macular corneal dystrophy Type I, Macular corneal dystrophy, type II, Microcornea, myopic chorioretinal atrophy, and telecanthus, Spinocerebellar ataxia, autosomal recessive, Cataract, multiple types, Ayme-gripp syndrome, Giant axonal neuropathy, Autoinflammation, antibody deficiency, and immune dysregulation, plcg2-associated, Ciliary dyskinesia, primary, Persistent fetal circulation, Keratoconus, Corneal fragility keratoglobus, blue sclerae AND joint hypermobility, Keratoconus, Granulomatous disease, chronic, autosomal recessive, cytochrome b-negative, Chronic granulomatous disease, Granulomatous disease, chronic, autosomal recessive, cytochrome b-negative, Lymphedema, hereditary, III, Adenine phosphoribosyltransferase deficiency, Mucopolysaccharidosis, MPS-IV-A, KBG syndrome, Astigmatism, Cryptorchidism, Hypertelorism, Esotropia, Retrognathia, Hypermetropia, Wide nasal bridge, Cryptorchidism, Epicanthus, Hypertelorism, Astigmatism, Intellectual disability, Global developmental delay, Fanconi anemia, complementation group A, Fanconi anemia, Cutaneous malignant melanoma, Malignant Melanoma Susceptibility, Ciliary dyskinesia, primary, Syndactyly type 9, Retinitis pigmentosa, Lissencephaly, Spongy degeneration of central nervous system, Spongy degeneration of central nervous system, Canavan Disease, Familial Form, Palmoplantar keratoderma, mutilating, with periorificial keratotic plaques, Nephropathic cystinosis, Cystinosis, atypical nephropathic, Myasthenic syndrome, congenital, 4a, slow-channel, Myasthenic syndrome, congenital, associated with acetylcholine receptor deficiency, Congenital myasthenic syndrome 1B, fast-channel, Pseudo von Willebrand disease, Amyotrophic lateral sclerosis, Combined oxidative phosphorylation deficiency, Leber congenital amaurosis, Orofaciodigital syndrome XV, Very long chain acyl-CoA dehydrogenase deficiency, Myasthenic syndrome, congenital, slow-channel, Li-Fraumeni syndrome, Hereditary cancer-predisposing syndrome, Familial colorectal cancer, Malignant lymphoma, non-Hodgkin, Liver cancer, Chronic lymphocytic leukemia, Medulloblastoma, Malignant melanoma of skin, Multiple myeloma, Squamous cell carcinoma of the head and neck, Small cell lung cancer, Lung adenocarcinoma, Squamous cell lung carcinoma, Acute myeloid leukemia, Neoplasm of brain, Neoplasm of the breast, Glioblastoma, Hepatocellular carcinoma, Hereditary cancer-predisposing syndrome, Pancreatic adenocarcinoma, Transitional cell carcinoma of the bladder, Brainstem glioma, Carcinoma of esophagus, Colorectal Neoplasms, Adrenocortical carcinoma, Adenocarcinoma of stomach, Ovarian Serous Cystadenocarcinoma, Malignant neoplasm of body of uterus, Adenocarcinoma of prostate, Uterine Carcinosarcoma, Metastatic pancreatic neuroendocrine tumours, Liver cancer, Chronic lymphocytic leukemia, Medulloblastoma, Malignant melanoma of skin, Multiple myeloma, Squamous cell carcinoma of the head and neck, Small cell lung cancer, Lung adenocarcinoma, Squamous cell lung carcinoma, Acute myeloid leukemia, Neoplasm of brain, Neoplasm of the breast, Glioblastoma, Hepatocellular carcinoma, Hereditary cancer-predisposing syndrome, Pancreatic adenocarcinoma, Transitional cell carcinoma of the bladder, Brainstem glioma, Carcinoma of esophagus, Colorectal Neoplasms, Adrenocortical carcinoma, Adenocarcinoma of stomach, Ovarian Serous Cystadenocarcinoma, Malignant neoplasm of body of uterus, Adenocarcinoma of prostate, Uterine Carcinosarcoma, Medulloblastoma, Multiple myeloma, Squamous cell carcinoma of the head and neck, Li-Fraumeni syndrome, Lung adenocarcinoma, Renal cell carcinoma, papillary, Neoplasm of the breast, Hereditary cancer-predisposing syndrome, Pancreatic adenocarcinoma, Squamous cell carcinoma of the skin, Transitional cell carcinoma of the bladder, Colorectal Neoplasms, Adenocarcinoma of stomach, Ovarian Serous Cystadenocarcinoma, Malignant neoplasm of body of uterus, Hereditary cancer-predisposing syndrome, Carcinoma of cervix, Liver cancer, Li-Fraumeni syndrome, Hepatocellular carcinoma, Hereditary cancer-predisposing syndrome, Liver cancer, Squamous cell carcinoma of the head and neck, Li-Fraumeni syndrome, Lung adenocarcinoma, Li-Fraumeni syndrome, Squamous cell lung carcinoma, Neoplasm of brain, Neoplasm of the breast, Glioblastoma, Hepatocellular carcinoma, Hereditary cancer-predisposing syndrome, Pancreatic adenocarcinoma, Transitional cell carcinoma of the bladder, Brainstem glioma, Carcinoma of esophagus, Colorectal Neoplasms, Adenocarcinoma of stomach, Ovarian Serous Cystadenocarcinoma, Adenocarcinoma of prostate, Uterine Carcinosarcoma, Liver cancer, Chronic lymphocytic leukemia, Multiple myeloma, Squamous cell carcinoma of the head and neck, Lung adenocarcinoma, Li-Fraumeni syndrome, Neoplasm of brain, Neoplasm of the breast, Glioblastoma, Hepatocellular carcinoma, Pancreatic adenocarcinoma, Transitional cell carcinoma of the bladder, Carcinoma of esophagus, Colorectal Neoplasms, Uterine cervical neoplasms, Adenocarcinoma of stomach, Ovarian Serous Cystadenocarcinoma, Malignant neoplasm of body of uterus, Uterine Carcinosarcoma, Li-Fraumeni syndrome, Liver cancer, Hepatocellular carcinoma, Hereditary cancer-predisposing syndrome, Liver cancer, Malignant melanoma of skin, Multiple myeloma, Squamous cell carcinoma of the head and neck, Lung adenocarcinoma, Breast cancer, somatic, Squamous cell lung carcinoma, Neoplasm of brain, Neoplasm of the breast, Hepatocellular carcinoma, Breast adenocarcinoma, Hereditary cancer-predisposing syndrome, Pancreatic adenocarcinoma, Transitional cell carcinoma of the bladder, Carcinoma of esophagus, Colorectal Neoplasms, Adenoid cystic carcinoma, Adenocarcinoma of stomach, Ovarian Serous Cystadenocarcinoma, Malignant neoplasm of body of uterus, Uterine Carcinosarcoma, Carcinoma of pancreas, Dyskeratosis congenita, autosomal recessive, Leber congenital amaurosis, Cone-rod dystrophy, Autosomal recessive congenital ichthyosis, Ichthyosis, Autosomal recessive congenital ichthyosis, Spondylocostal dysostosis, Inclusion Body Myopathy, Dominant, Hepatic failure, early-onset, and neurologic disorder due to cytochrome C oxidase deficiency, Charcot-Marie-Tooth disease and deafness, Dejerine-Sottas disease, Dejerine-Sottas disease, Dejerine-Sottas syndrome, autosomal dominant, Charcot-Marie-Tooth disease, type IA, Dejerine-Sottas syndrome, autosomal dominant, Charcot-Marie-Tooth disease, type I, Mitochondrial complex III deficiency, nuclear type 2, Common variable immunodeficiency, Immunoglobulin A deficiency, Common Variable Immune Deficiency, Dominant, Common variable immunodeficiency, Hereditary cancer-predisposing syndrome, Multiple fibrofolliculomas, Hereditary cancer-predisposing syndrome, Hereditary cancer-predisposing syndrome, Multiple fibrofolliculomas, Hereditary cancer-predisposing syndrome, Smith-Magenis syndrome, Joubert syndrome, Meckel-Gruber syndrome, Sjögren-Larsson syndrome, Congenital disorders of glycosylation type II, Congenital disorder of glycosylation IIp, Congenital defect of folate absorption, Immunodeficiency, Cone-Rod Dystrophy, Dominant, Neurofibromatosis, type 1, Hereditary cancer-predisposing syndrome, Breast-ovarian cancer, familial 4, Hereditary cancer-predisposing syndrome, Infantile Refsum's disease, Peroxisome biogenesis disorders, Zellweger syndrome spectrum, Peroxisome biogenesis disorder, Familial hypoplastic, glomerulocystic kidney, Limb-girdle muscular dystrophy, type 2G, Hyperphosphatasia with mental retardation syndrome, Neoplasm of the breast, Transitional cell carcinoma of the bladder, Carcinoma of esophagus, Uterine cervical neoplasms, Adenocarcinoma of stomach, Neoplasm of the breast, Colorectal Neoplasms, Adenocarcinoma of stomach, Hypothyroidism, congenital, nongoitrous, Autosomal recessive woolly hair, Autosomal Recessive Hypotrichosis with Woolly Hair, Bullous ichthyosiform erythroderma, Meesman's corneal dystrophy, Dermatopathia pigmentosa reticularis, Naxos disease, Ciliary dyskinesia, primary, Autoimmune disease, multisystem, infantile-onset, Mucopolysaccharidosis, MPS-III-B, Glycogen storage disease type 1A, Breast-ovarian cancer, familial, Breast-ovarian cancer, familial, Hereditary breast and ovarian cancer syndrome, Breast-ovarian cancer, familial 1, Hereditary breast and ovarian cancer syndrome, Hereditary cancer-predisposing syndrome, Familial cancer of breast, Hereditary cancer-predisposing syndrome, Breast-ovarian cancer, familial, Hereditary breast and ovarian cancer syndrome, Breast-ovarian cancer, familial, Familial cancer of breast, Breast-ovarian cancer, familial 1, Hereditary breast and ovarian cancer syndrome, Neoplasm of the breast, Hereditary cancer-predisposing syndrome, Breast-ovarian cancer, familial, Hereditary breast and ovarian cancer syndrome, Hereditary cancer-predisposing syndrome, Breast-ovarian cancer, familial, Hereditary breast and ovarian cancer syndrome, Hereditary breast and ovarian cancer syndrome, Hereditary cancer-predisposing syndrome, Familial cancer of breast, Breast-ovarian cancer, familial, Hereditary breast and ovarian cancer syndrome, Hereditary cancer-predisposing syndrome, Neoplasm of the breast, Renal tubular acidosis, autosomal dominant, Frontotemporal dementia, ubiquitin-positive, Growth and mental retardation, mandibulofacial dysostosis, microcephaly, and cleft palate, Alexander's disease, Progressive supranuclear ophthalmoplegia, Frontotemporal dementia, Progressive supranuclear ophthalmoplegia, Muscular dystrophy, Epilepsy, progressive myoclonic 6, Glanzmann thrombasthenia, Amelogenesis imperfecta, type IV, Tricho-dento-osseous syndrome, Osteogenesis imperfecta type I, Osteogenesis imperfecta type 2, thin-bone, Osteogenesis imperfecta with normal sclerae, dominant form, Osteogenesis imperfecta type I, Osteogenesis imperfecta type IIC, Osteogenesis imperfecta, recessive perinatal lethal, Osteogenesis imperfecta type I, Osteogenesis imperfecta with normal sclerae, dominant form, Osteogenesis imperfecta type III, Osteogenesis imperfecta, type III/iv, Osteogenesis imperfecta, recessive perinatal lethal, Osteogenesis imperfecta with normal sclerae, dominant form, Osteogenesis imperfecta type 1, mild, Proximal symphalangism, Tarsal carpal coalition syndrome, Joubert syndrome, Joubert syndrome, Fanconi anemia, complementation group O, Hereditary cancer-predisposing syndrome, Fanconi anemia, complementation group O, Retinitis pigmentosa, Ischiopatellar dysplasia, Familial cancer of breast, Fanconi anemia, complementation group J, Neoplasm of ovary, Hereditary breast and ovarian cancer syndrome, Hereditary cancer-predisposing syndrome, Hereditary cancer-predisposing syndrome, Familial cancer of breast, Fanconi anemia, complementation group J, Hereditary cancer-predisposing syndrome, Hereditary breast and ovarian cancer syndrome, Hereditary cancer-predisposing syndrome, Rolandic epilepsy, Isolated growth hormone deficiency type 1B, Hyperkalemic Periodic Paralysis Type 1, Potassium aggravated myotonia, Paramyotonia congenita of von Eulenburg, Paramyotonia congenita/hyperkalemic periodic paralysis, Hyperkalemic Periodic Paralysis Type 1, Hypokalemic periodic paralysis, Hypokalemic periodic paralysis, type 2, Hyperkalemic Periodic Paralysis Type 1, Carcinoma of colon, Oligodontia-colorectal cancer syndrome, Carney complex, type 1, Andersen Tawil syndrome, Familial periodic paralysis, Andersen Tawil syndrome, Andersen Tawil syndrome, Congenital long QT syndrome, Acampomelic campomelic dysplasia, Camptomelic dysplasia, Striatal necrosis, bilateral, and progressive polyneuropathy, Pontocerebellar hypoplasia type 4, Pontocerebellar hypoplasia type 2A, Pontocerebellar hypoplasia type 4, Pontocerebellar hypoplasia type 2A, Pontocerebellar hypoplasia type 5, Congenital cerebellar hypoplasia, Hypertonia, Microcephaly, Amblyopia, Global developmental delay, Olivopontocerebellar hypoplasia, Non-syndromic pontocerebellar hypoplasia, Olivopontocerebellar hypoplasia, Deficiency of galactokinase, Hemophagocytic lymphohistiocytosis, familial, Pseudoneonatal adrenoleukodystrophy, Epidermodysplasia verruciformis, Desbuquois dysplasia, Rolandic epilepsy, Ciliary dyskinesia, Ciliary dyskinesia, primary, Glycogen storage disease, type II, Glycogen storage disease type II, infantile, Glycogen storage disease, type II, Baraitser-Winter Syndrome, Nephrotic syndrome, type 8, Autosomal recessive cutis laxa type 2B, Encephalopathy, progressive, early-onset, with brain atrophy and thin corpus callosum, Arhinia choanal atresia microphthalmia, Oculomelic amyoplasia, Dystonia, Spinocerebellar ataxia, ACTH resistance, Glucocorticoid Deficiency, Renal hypodysplasia/aplasia, Left ventricular noncompaction, Pancreatic agenesis and congenital heart disease, Abnormality of cardiovascular system morphology, Congenital diaphragmatic hernia, Seckel syndrome, Niemann-Pick disease type C1, Niemann-Pick disease type C1, Niemann-Pick disease, type D, Scalp ear nipple syndrome, Arrhythmogenic right ventricular cardiomyopathy, type 10, Arrhythmogenic right ventricular cardiomyopathy, Cardiovascular phenotype, Arrhythmogenic right ventricular cardiomyopathy, type 10, Amyloidogenic transthyretin amyloidosis, Cardiovascular phenotype, Bainbridge-Ropers syndrome, Mental retardation, autosomal recessive, Vici syndrome, Carcinoma of pancreas, Juvenile polyposis syndrome, Colorectal Neoplasms, Juvenile polyposis/hereditary hemorrhagic telangiectasia syndrome, Juvenile polyposis syndrome, Colorectal Neoplasms, Mirror movements, Carcinoma of colon, Pitt-Hopkins syndrome, Erythropoietic protoporphyria, Progressive intrahepatic cholestasis, Periventricular nodular heterotopia with syndactyly, cleft palate and developmental delay, Periventricular nodular heterotopia, Immunodeficiency, Obesity, Schizophrenia, Obesity, Osteopetrosis autosomal recessive, Burn-McKeown syndrome, Severe congenital neutropenia autosomal dominant, Cyclical neutropenia, Complement factor d deficiency, Spondylometaphyseal dysplasia Sedaghatian type, Carcinoma of pancreas, Peutz-Jeghers syndrome, Hereditary cancer-predisposing syndrome, Hereditary cancer-predisposing syndrome, Cutaneous malignant melanoma, Cutaneous melanoma, Persistent mullerian duct syndrome, type I, Preimplantation embryonic lethality, Hypocalcemia, autosomal dominant, Cone-rod dystrophy, Age-related macular degeneration, Spinocerebellar ataxia, Cardiofaciocutaneous syndrome, Cardio-facio-cutaneous syndrome, CODAS syndrome, Leukodystrophy, hypomyelinating, Insulin-resistant diabetes mellitus and acanthosis nigricans, Pineal hyperplasia and diabetes mellitus syndrome, Insulin-resistant diabetes mellitus and acanthosis nigricans, Leprechaunism syndrome, Pineal hyperplasia and diabetes mellitus syndrome, Retinitis pigmentosa, Mucolipidosis type IV, Mucolipidosis type IV, Mucolipidosis type IV, Boucher Neuhauser syndrome, Weill-Marchesani syndrome, Cerebellar ataxia, deafness and narcolepsy, autosomal dominant, Tyrosine kinase 2 deficiency, Charcot-Marie-Tooth disease, type 2M, Familial hypercholesterolemia, Familial hypercholesterolemias, Kartagener syndrome, Ciliary dyskinesia, primary, Spondyloenchondrodysplasia with immune dysregulation, Deficiency of alpha-mannosidase, Aicardi Goutieres syndrome, Blood group—Lutheran inhibitor, Glutaric aciduria, type 1, Marshall-Smith syndrome, Epileptic encephalopathy, early infantile, Familial hemiplegic migraine type 1, Episodic ataxia type 2, Epileptic encephalopathy, early infantile, Familial hemiplegic migraine type 1, Autosomal recessive non-syndromic intellectual disability, Lehman syndrome, Cerebral autosomal dominant arteriopathy with subcortical infarcts and leukoencephalopathy, Combined oxidative phosphorylation deficiency, Severe combined immunodeficiency, autosomal recessive, T cell-negative, B cell-positive, NK cell-negative, Thyroid dyshormonogenesis, Cold-induced sweating syndrome, Pseudoachondroplastic spondyloepiphyseal dysplasia syndrome, Multiple epiphyseal dysplasia, Pseudoachondroplastic spondyloepiphyseal dysplasia syndrome, Epiphyseal dysplasia, multiple, severe, Bilateral right-sidedness sequence, Transposition of the great arteries, dextro-looped, Heterotaxia, Acute myeloid leukemia, Arthrogryposis multiplex congenita, neurogenic, with myelin defect, Hemochromatosis type 1, Dystonia 28, childhood-onset, Finnish congenital nephrotic syndrome, Central core disease, Central core disease, Malignant hyperthermia, susceptibility to, RYR1-Related Disorders, Congenital myopathy with fiber type disproportion, RYR1-Related Disorders, Myopathy, Congenital myopathy with fiber type disproportion, Central core disease, Malignant hyperthermia, susceptibility to, Central core disease, Congenital myopathy with fiber type disproportion, Central core disease, Cutis laxa with severe pulmonary, gastrointestinal, and urinary abnormalities, Nephrotic syndrome, type 9, Maple syrup urine disease, Diamond-Blackfan anemia, Alternating hemiplegia of childhood, Dystonia, Familial partial lipodystrophy 6, Ethylmalonic encephalopathy, Blood group—Lutheran Null, Familial type 3 hyperlipoproteinemia, Apolipoprotein C2 deficiency, Apolipoprotein C-II (Padova), Apolipoprotein C2 deficiency, Apolipoprotein C-II (Auckland), Immunodeficiency, Hermansky-Pudlak syndrome, Xeroderma pigmentosum, group D, Trichothiodystrophy, photosensitive, Congenital muscular dystrophy-dystroglycanopathy with mental retardation, type B5, Muscular dystrophy-dystroglycanopathy (congenital with brain and eye anomalies), type a, Congenital muscular dystrophy-dystroglycanopathy (with or without mental retardation) type B5, Limb-girdle muscular dystrophy-dystroglycanopathy, type C5, Limb-girdle muscular dystrophy, Congenital muscular dystrophy-dystroglycanopathy with brain and eye anomalies type A5, Muscle weakness, Headache, Gait imbalance, Difficulty walking, Paresthesia, Difficulty climbing stairs, Scapular winging, Difficulty standing, Muscular dystrophy-dystroglycanopathy, Walker-Warburg congenital muscular dystrophy, Congenital muscular dystrophy-dystroglycanopathy with mental retardation, type B5, Limb-girdle muscular dystrophy-dystroglycanopathy, type C5, Walker-Warburg congenital muscular dystrophy, Walker-Warburg congenital muscular dystrophy, Congenital muscular dystrophy-dystroglycanopathy without mental retardation, type B5, Walker-Warburg congenital muscular dystrophy, Congenital muscular dystrophy-dystroglycanopathy with mental retardation, type B5, Limb-girdle muscular dystrophy-dystroglycanopathy, type C5, Congenital muscular dystrophy-dystroglycanopathy with mental retardation, type B5, Congenital muscular dystrophy-dystroglycanopathy with brain and eye anomalies type A5, Walker-Warburg congenital muscular dystrophy, Hypocalciuric hypercalcemia, familial, type III, Mental retardation, autosomal recessive, Hyperferritinemia cataract syndrome, L-ferritin deficiency, autosomal recessive, Isolated lutropin deficiency, Autistic disorder of childhood onset, Motor delay, Iris coloboma, Autism, Delayed speech and language development, Abnormality of vision, Early infantile epileptic encephalopathy, Ataxia-oculomotor apraxia, Early infantile epileptic encephalopathy, Peripheral neuropathy, myopathy, hoarseness, and hearing loss, Spinocerebellar ataxia, Spinocerebellar ataxia, Retinitis pigmentosa, Nemaline myopathy, Polyglucosan body myopathy with or without immunodeficiency, Glycogen storage disease, type IV, Brown-Vialetto-Van Laere syndrome, Spinocerebellar ataxia, Cerebro-costo-mandibular syndrome, Neurohypophyseal diabetes insipidus, Pigmentary pallidal degeneration, Hypoprebetalipoproteinemia, acanthocytosis, retinitis pigmentosa, and pallidal degeneration, Pigmentary pallidal degeneration, Spongiform encephalopathy with neuropsychiatric features, Genetic prion diseases, Gerstmann-Straussler-Scheinker syndrome, Cerebral Amyloid Angiopathy, PRNP-related, Ataxia-telangiectasia-like disorder, Kindler's syndrome, Short stature, facial dysmorphism, and skeletal anomalies with or without cardiac anomalies, Auriculocondylar syndrome, McKusick Kaufman syndrome, Alagille syndrome, Mitochondrial complex I deficiency, Leigh syndrome, Congenital dyserythropoietic anemia, type II, Cowden syndrome, Congenital dyserythropoietic anemia, Retinitis pigmentosa, Otofaciocervical syndrome, Thrombophilia due to thrombomodulin defect, Thrombophilia due to thrombomodulin defect, Joint laxity, short stature, and myopia, Craniofacial anomalies and anterior segment dysgenesis syndrome, Familial hypertrophic cardiomyopathy, Cardiomyopathy, hypertrophic, midventricular, digenic, Dowling-Degos disease, C-like syndrome, Multiple synostoses syndrome, Symphalangism, proximal, Fibular hypoplasia and complex brachydactyly, schizophrenia, Aicardi Goutieres syndrome, Severe combined immunodeficiency due to ADA deficiency, Partial adenosine deaminase deficiency, Multiple congenital anomalies-hypotonia-seizures syndrome, Primary autosomal recessive microcephaly, Galloway-Mowat Syndrome, Arterial tortuosity syndrome, Epileptic encephalopathy, early infantile, Helsmoortel-van der aa syndrome, Congenital disorder of glycosylation type 1E, Idiopathic hypercalcemia of infancy, Cushing's syndrome, McCune-Albright syndrome, Polyostotic fibrous dysplasia, somatic, mosaic, Pituitary Tumor, Growth Hormone-Secreting, Somatic, Liver cancer, McCune-Albright syndrome, Malignant melanoma of skin, Squamous cell carcinoma of the head and neck, Lung adenocarcinoma, Neoplasm of the breast, Hepatocellular carcinoma, Pancreatic adenocarcinoma, Neoplasm, Colorectal Neoplasms, Uterine cervical neoplasms, Adrenocortical carcinoma, Adenocarcinoma of stomach, McCune-Albright syndrome, Pseudohypoparathyroidism, type IA, with testotoxicosis, Pseudohypoparathyroidism type 1C, Waardenburg syndrome type 4B, Early infantile epileptic encephalopathy, Benign familial neonatal seizures, Early infantile epileptic encephalopathy, Seizures, Generalized hypotonia, Early infantile epileptic encephalopathy, Benign familial neonatal seizures, Dyskeratosis congenita, autosomal recessive, Pulmonary fibrosis and/or bone marrow failure, telomere-related, Dyskeratosis congenita, Dyskeratosis congenita, autosomal recessive, Dyskeratosis congenita, autosomal recessive, Glomerulonephritis with sparse hair and telangiectases, Alzheimer disease, type 1, Amyotrophic lateral sclerosis type 1, Inflammatory bowel disease, autosomal recessive, Immunodeficiency, Familial platelet disorder with associated myeloid malignancy, Familial platelet disorder with associated myeloid malignancy, Transient myeloproliferative disorder of Down syndrome, Leukemia, acute myeloid, m0 subtype, Popliteal pterygium syndrome lethal type, Kartagener syndrome, Primary ciliary dyskinesia, Kartagener syndrome, Ciliary dyskinesia, Primary ciliary dyskinesia, Homocystinuria due to CBS deficiency, Epileptic encephalopathy, early infantile, Unverricht-Lundborg syndrome, Autoimmune polyglandular syndrome type 1, autosomal dominant, Leukocyte adhesion deficiency type 1, Bethlem myopathy, Ullrich congenital muscular dystrophy, Ullrich congenital muscular dystrophy, Microcephalic osteodysplastic primordial dwarfism type 2, Polyarteritis nodosa, childhoood-onset, Peroxisome biogenesis disorder, Proline dehydrogenase deficiency, Schizophrenia, Autosomal recessive Noonan-like syndrome due to compound heterozygous variants in LZTR1, Spinal muscular atrophy, jokela type, Frontotemporal dementia and/or amyotrophic lateral sclerosis, Myopathy, isolated mitochondrial, autosomal dominant, Rhabdoid tumor predisposition syndrome, Schwannomatosis, Deficiency of beta-ureidopropionase, Congenital cataract, Klippel-feil syndrome, autosomal recessive, with nemaline myopathy and facial dysmorphism, Hermansky-Pudlak syndrome, Cataract, congenital nuclear, autosomal recessive, Cataract, multiple types, Familial cancer of breast, Hereditary cancer-predisposing syndrome, Hereditary cancer-predisposing syndrome, Familial cancer of breast, Prostate cancer, somatic, Hereditary cancer-predisposing syndrome, Osteosarcoma, Neurofibromatosis, type 2, Epilepsy, familial focal, with variable foci, Rolandic epilepsy, Parkinson disease, Sorsby fundus dystrophy, Macrothrombocytopenia and granulocyte inclusions with or without nephritis or sensorineural hearing loss, Microcytic anemia, Peripheral demyelinating neuropathy, central dysmyelination, Waardenburg syndrome, and Hirschsprung disease, Waardenburg syndrome type 4C, Parkinson disease, Infantile neuroaxonal dystrophy, Adenylosuccinate lyase deficiency, Nephronophthisis-like nephropathy, Carcinoma of colon, Rubinstein-Taybi syndrome, Carcinoma of colon, Kanzaki disease, Methemoglobinemia type 2, Autosomal recessive syndrome of syndactyly, undescended testes and central nervous system defects, Megalencephalic leukoencephalopathy with subcortical cysts, Microcephaly with chorioretinopathy, autosomal recessive, Mitochondrial DNA depletion syndrome (MNGIE type), Muscular dystrophy, congenital, megaconial type, Metachromatic leukodystrophy, juvenile type, Metachromatic leukodystrophy, late infantile, Metachromatic leukodystrophy, Metachromatic leukodystrophy, severe, Metachromatic leukodystrophy, Short stature, idiopathic, X-linked, Leri Weill dyschondrosteosis, Chondrodysplasia punctata, X-linked recessive, Kallmann syndrome, Ocular albinism, type I, Opitz-Frias syndrome, Amelogenesis imperfecta, type 1E, Spondyloepiphyseal dysplasia tarda, Oral-facial-digital syndrome, Joubert syndrome, Joubert syndrome, Oral-facial-digital syndrome, Paroxysmal nocturnal hemoglobinuria 1, Multiple congenital anomalies-hypotonia-seizures syndrome, Pettigrew syndrome, Nance-Horan syndrome, Congenital cataract, Early infantile epileptic encephalopathy, Early infantile epileptic encephalopathy, Atypical Rett syndrome, Early infantile epileptic encephalopathy, Angelman syndrome-like, Early infantile epileptic encephalopathy, Atypical Rett syndrome, Early infantile epileptic encephalopathy, Angelman syndrome-like, Early infantile epileptic encephalopathy, Atypical Rett syndrome, Early infantile epileptic encephalopathy, Angelman syndrome-like, Juvenile retinoschisis, Glycogen storage disease type IXa1, Coffin-Lowry syndrome, Deafness, X-linked, IFAP syndrome with or without BRESHECK syndrome, Familial X-linked hypophosphatemic vitamin D refractory rickets, Hydranencephaly with abnormal genitalia, Proud Levine Carpenter syndrome, Lissencephaly, X-linked, epileptic encephalopathy, early infanitle, Mental retardation, X-linked, Congenital adrenal hypoplasia, X-linked, Becker muscular dystrophy, Duchenne muscular dystrophy, Becker muscular dystrophy, Duchenne muscular dystrophy, Dilated cardiomyopathy, Granulomatous disease, chronic, X-linked, variant, Cone-rod dystrophy, X-linked, Retinitis pigmentosa, Ornithine carbamoyltransferase deficiency, Mental retardation, X-linked, Mental retardation, X-linked, Congenital stationary night blindness, type 1A, Mental retardation and microcephaly with pontine and cerebellar hypoplasia, FG syndrome, Monoamine oxidase A deficiency, Atrophia bulborum hereditaria, Familial exudative vitreoretinopathy, X-linked, Atrophia bulborum hereditaria, Kabuki syndrome, Retinitis pigmentosa, Arthrogryposis multiplex congenita, distal, X-linked, Properdin deficiency, X-linked, Chondrodysplasia punctata, X-linked dominant, atypical, Chondrodysplasia punctata X-linked dominant, MEND syndrome, Wiskott-Aldrich syndrome, GATA-1-related thrombocytopenia with dyserythropoiesis, Dyserythropoietic anemia with thrombocytopenia, GATA-1-related thrombocytopenia with dyserythropoiesis, Neurodegeneration with brain iron accululation, Nephrolithiasis, X-linked recessive, Dent disease, Mental retardation, syndromic, Claes-Jensen type, X-linked, 2-methyl-3-hydroxybutyric aciduria, Aarskog syndrome, Hereditary sideroblastic anemia, Amyotrophic lateral sclerosis, with or without frontotemporal dementia, Androgen resistance syndrome, Partial androgen insensitivity syndrome, Prostate cancer susceptibility, Androgen resistance syndrome, Partial androgen insensitivity syndrome, Craniofrontonasal dysplasia, Hypohidrotic X-linked ectodermal dysplasia, Hypohidrotic ectodermal dysplasia, Tooth agenesis, selective, X-linked, Myopia, X-Linked, Female-Limited, X-linked severe combined immunodeficiency, Ohdo syndrome, X-linked, FG syndrome, Intellectual functioning disability, Cardiovascular phenotype, X-linked hereditary motor and sensory neuropathy, Mental retardation, X-linked, syndromic, Mental Retardation, X-Linked, Cornelia de Lange syndrome 5, Glycogen storage disease, Allan-Herndon-Dudley syndrome, Mental retardation, X-linked, Metacarpal 4-5 fusion, ATR-X syndrome, Menkes kinky-hair syndrome, Menkes kinky-hair syndrome, Cutis laxa, X-linked, Distal spinal muscular atrophy, X-linked, Phosphoglycerate kinase 1 deficiency, Cleft palate with ankyloglossia, Mental retardation, X-linked, Choroideremia, Early infantile epileptic encephalopathy, Mohr-Tranebjaerg syndrome, X-linked agammaglobulinemia, Agammaglobulinemia, non-Bruton type, Fabry disease, Fabry disease, Deoxygalactonojirimycin response, Pelizaeus-Merzbacher disease, Pelizaeus-Merzbacher disease, connatal, Thyroxine-binding globulin, variant P, Phosphoribosylpyrophosphate synthetase superactivity, Charcot-Marie-Tooth disease, X-linked recessive, type 5, Alport syndrome, X-linked recessive, Microscopic hematuria, Elevated mean arterial pressure, Chronic kidney disease, Mental retardation, X-linked, Megalocornea, Mental retardation, X-linked, Heterotopia, Lissencephaly, X-linked, Fucosidosis, Lissencephaly, X-linked, Subcortical laminar heterotopia, X-linked, Danon disease, Syndromic X-linked mental retardation, Cabezas type, Mental retardation, X-linked, syndromic, wu type, Lymphoproliferative syndrome, X-linked, Lymphoproliferative syndrome, X-linked, Simpson-Golabi-Behmel syndrome, Borjeson-Forssman-Lehmann syndrome, Lesch-Nyhan syndrome, Lesch-Nyhan syndrome, HPRT Flint, Partial hypoxanthine-guanine phosphoribosyltransferase deficiency, HPRT Munich, HPRT Milwaukee, Lesch-Nyhan syndrome, Christianson syndrome, Hypertrophic cardiomyopathy, Myopathy, reducing body, X-linked, early-onset, severe, Immunodeficiency with hyper IgM type 1, Pituitary adenoma, growth hormone-secreting, Heterotaxy, visceral, X-linked, VACTERL association with hydrocephaly, X-linked, Congenital heart defects, nonsyndromic, Heterotaxy, visceral, X-linked, Hereditary factor IX deficiency disease, Hereditary factor IX deficiency disease, Thrombophilia, X-linked, due to factor IX defect, Mucopolysaccharidosis, MPS-II, Mucopolysaccharidosis, type II, severe form, Mucopolysaccharidosis, MPS-II, Hypospadias, X-linked, Severe X-linked myotubular myopathy, Child syndrome, Spondyloepimetaphyseal dysplasia X-linked, Microcephaly, Carious teeth, Intellectual disability, Global developmental delay, Abnormality of the cerebral cortex, Skeletal muscle atrophy, Oral-pharyngeal dysphagia, Muscular hypotonia, Muscular hypotonia, Creatine deficiency, X-linked, Chromosome Xq28 deletion syndrome, Adrenoleukodystrophy, Nephrogenic diabetes insipidus, X-linked, Nephrogenic syndrome of inappropriate antidiuresis, N-terminal acetyltransferase deficiency, Rett syndrome, Mental retardation, X-linked, syndromic, Rett syndrome, Rett syndrome, Stereotypy, Delayed speech and language development, Delayed gross motor development, Bruxism, Deuteranopia, Otopalatodigital spectrum disorder, Melnick-Needles syndrome, Periventricular nodular heterotopia, Melnick-Needles syndrome, Oto-palato-digital syndrome, type II, Frontometaphyseal dysplasia, Cardiac valvular dysplasia, X-linked, Periventricular nodular heterotopia, Oto-palato-digital syndrome, type II, Oto-palato-digital syndrome, type I, Emery-Dreifuss muscular dystrophy, X-linked, 3-Methylglutaconic aciduria type 2, Galloway-Mowat Syndrome, X-Linked, Glucose 6 phosphate dehydrogenase deficiency, G6pd a-, G6PD Canton, G6PD GIFU, G6PD Agrigento, G6PD Taiwan-Hakka, Anemia, nonspherocytic hemolytic, due to G6PD deficiency, G6PD LOMA Linda, Anemia, nonspherocytic hemolytic, due to G6PD deficiency, Glucose phosphate dehydrogenase deficiency, G6pd a-G6PD Gastonia, G6PD Marion, G6PD Minnesota, Anemia, nonspherocytic hemolytic, due to G6PD deficiency, Hypohidrotic ectodermal dysplasia with immune deficiency, Dyskeratosis congenita X-linked, Hereditary factor VIII deficiency disease, Parkinsonism, early onset with mental retardation, Mental retardation, X-linked, Leri Weill dyschondrosteosis, XY sex reversal, type 1, Leigh syndrome, Chloramphenicol resistance, nonsyndromic sensorineural, mitochondrial, Leber's optic atrophy, Cytochrome c oxidase i deficiency, Leigh syndrome, Mitochondrial complex I deficiency, Leigh syndrome, Retinitis pigmentosa-deafness syndrome, Cerebellar ataxia, cataract, and diabetes mellitus.

Pathogenic T to G or A to C mutations may be corrected using the methods and compositions provided herein, for example by mutating the C to a T, and/or the G to an A, and thereby restoring gene function. Guide RNAs (gRNA) sequences, which encode RNA that can direct a napDNAbp, or any of the base editors provided herein, to a target site gRNA sequences may be cloned into an expression vector, such as Addgene pFYF1320 (which targets EGFP), to encode a gRNA that targets a napDNAbp, or any of the base editors provided herein, to a target site in order to correct a disease-related mutation.

In some aspects, the present disclosure provides uses of any one of the base editors described or evaluated by the systems herein, and a guide RNA targeting this base editor to a target C:G base pair in a nucleic acid molecule, in the manufacture of a kit for base editing, wherein the base editing comprises contacting the nucleic acid molecule with the base editor and guide RNA under conditions suitable for the substitution of the cytosine (C) of the C:G nucleobase pair with a thymine (T). In some embodiments of these uses, the nucleic acid molecule is a double-stranded DNA molecule. In some embodiments, the step of contacting of induces separation of the double-stranded DNA at a target region. In some embodiments, the step of contacting further comprises nicking one strand of the double-stranded DNA, wherein the one strand comprises an unmutated strand that comprises the G of the target C:G nucleobase pair.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in a cell, such as a human or non-human animal cell.

The present disclosure also provides uses of any one of the base editors described herein as a medicament. The present disclosure also provides uses of any one of the complexes of base editors and guide RNAs described herein as a medicament.

### Multiplexed Base Editing Applications

In some aspects, the present disclosure provides methods of editing multiple nucleic acid target sites using the disclosed cytosine base editors. In multiplexed base editing of unique genomic loci, a plurality of gRNAs having complementarity to different target sequences enables the formation of fusion protein-gRNA complexes at each of several (e.g. 5, 10, 15, 20, 25, or more) target sequences simulataneously, or within a single iteration or cycle.

The discovery and widespread implementation of the CRISPR/Cas system has dramatically expanded the toolbox for genome engineering and has revolutionized the future prospects of basic biological research, data storage in living systems, agricultural science, and medicine. An advantage of CRISPR/Cas-based genome editors over prior approaches is the capacity to multiplex by using several guide RNAs (gRNAs). This not only enables the screening of libraries of guides in a single cell population but also the targeting of up to six unique loci at once. However, the editing efficiency at each site tends to decrease when compared to that of a single guide transfection.

The present disclosure provides for methods of base editing comprising: contacting a nucleic acid molecule (e.g. DNA) with a plurality of complexes, wherein each complex comprises a base editor and a guide RNA (gRNA) bound to the napDNAbp domain of the base editor, wherein at least five of the fusion proteins of the plurality are each bound to a unique gRNA comprising a different guide sequence of at least 5, 7, or 10 contiguous nucleotides that is complementary to a target sequence in the genomic DNA of a eukaryotic cell. In certain embodiments, the plurality of the disclosed fusion protein-gRNA complexes make simultaneous edits (i.e., within a single iteration) at various target loci within a eukaryotic cell, e.g. a mammalian cell.

In some embodiments, the deamination efficiency at each unique genomic loci is substantially equivalent to that of a single guide transfection at each of these loci.

Any of the base editor-gRNA complexes provided herein may be introduced into the cell for multiplexed base editing in any suitable way, either stably or transiently. In some embodiments, a fusion protein may be transfected into the cell. In some embodiments, the cell may be transduced or transfected with a nucleic acid construct that encodes the fusion protein. For example, a cell may be transduced (e.g. with a virus encoding a fusion protein) or transfected (e.g. with a plasmid encoding a fusion protein) with a nucleic acid that encodes the fusion protein. Alternatively, a cell may be introduced with the fusion protein itself. Such transduction may be a stable or transient transduction. In some embodiments, cells expressing a base editing fusion protein, or comprising a fusion protein, may be transduced or transfected with one or more gRNA molecules, for example, when the fusion protein comprises a Cas9 (e.g. dCas9) domain. In some embodiments, a plasmid expressing a fusion protein may be introduced into cells through electroporation, transient (e.g. lipofection) or stable genome integration (e.g. piggybac), viral transduction, or other methods known to those of skill in the art.

In certain embodiments of the disclosed methods, the constructs that encode the fusion proteins are transfected into the cell separately from the constructs that encode the gRNAs. In certain embodiments, these components are encoded on a single construct and transfected together. In particular embodiments, these single constructs encoding the fusion proteins and gRNAs may be transfected into the cell iteratively, with each iteration associated with a subset of target sequences. In particular embodiments, these single constructs may be transfected into the cell over a period of days. In other embodiments, they may be transfected into the cell over a period of hours. In other embodiments, they may be transected into the cell over a period of weeks.

In the disclosed methods, target cells may be incubated with the fusion protein-gRNA complexes for two days, or 48 hours, after transfection to achieve multiplexed base editing. Target cells may be incubated for 30 hours, 40 hours, 54 hours, 60 hours, or 72 hours after transfection. Target cells may be incubated with the fusion protein-gRNA complexes for four days, five days, seven days, nine days, eleven days, or thirteen days or more after transfection.

In various embodiments, the step of contacting results in a base editing efficiency of at least about 35%, 40%, 50%, 60%, 70%, 80%, 85%, 90%, 95%, 98%, or 99%. The step of contacting may result in in a base editing efficiency of at least about 51%, 52%, 53%, 54%, 55%, 56% or 57%. In particular, the step of contacting results in base editing efficiencies of greater than 54%. In certain embodiments, base editing efficiencies of 99% may be realized.

In some aspects, the disclosure provides pharmaceutical compositions comprising a plurality of any of the base editors described herein and a gRNA, wherein at least five of the base editors of the plurality are each bound to a unique gRNA, and a pharmaceutically acceptable excipient.

### Pharmaceutical Compositions

Other aspects of the present disclosure relate to pharmaceutical compositions comprising any of the base editors or base editor-gRNA complexes described herein (e.g., including, but not limited to, the napDNAbps, base editors, guide RNAs, and complexes comprising base editors and guide RNAs).

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, for targeted delivery, increasing half-life, or other therapeutic compounds).

In some embodiments, any of the base editors, gRNAs, and/or complexes described herein are provided as part of a pharmaceutical composition. In some embodiments, the pharmaceutical composition comprises any of the base editors provided herein. In some embodiments, the pharmaceutical composition comprises any of the complexes provided herein. In some embodiments pharmaceutical composition comprises a gRNA, a base editor, and a pharmaceutically acceptable excipient. Pharmaceutical compositions may optionally comprise one or more additional therapeutically active substances.

In some embodiments, compositions provided herein are formulated for delivery to a subject, for example, to a human subject, in order to effect a targeted genomic modification within the subject. In some embodiments, cells are obtained from the subject and contacted with a any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; 7,163,824, 9,526,784; 9,737,604; and U.S. Patent Publication Nos. 2018-0127780, published May 10, 2018, and 2018-0236081, published Aug. 23, 2018, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated in its entirety herein by reference) discloses various excipients used in formulating pharmaceutical compositions and known techniques for the preparation thereof. See also PCT application PCT/US2010/055131, filed Nov. 2, 2010 (Publication No. WO 2011/053982, published May 5, 2011), incorporated in its entirety herein by reference, for additional suitable methods, reagents, excipients and solvents for producing pharmaceutical compositions comprising a nuclease. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

As used herein, the term “pharmaceutically acceptable carrier” means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants may also be present in the formulation. The terms such as “excipient”, “carrier”, “pharmaceutically acceptable carrier” or the like are used interchangeably herein.

Suitable routes of administering the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., a tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; 4,921,757; 9,526,784, 9,737,604; and U.S. Patent Publication No. 2018-0127780, published May 10, 2018, each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Delivery Methods

In some aspects, the disclosure provides methods comprising delivering any of the base editors, gRNAs, and/or complexes described herein. In other embodiments, the disclosure provides methods comprising delivery of one or more vectors as described herein, one or more transcripts thereof, and/or one or proteins transcribed therefrom, to a host cell. In some embodiments, the invention further provides cells produced by such methods, and organisms (such as animals, plants, or fungi) comprising or produced from such cells. In some embodiments, a base editor as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include ribonucleoprotein (RNP) complexes, DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

In some embodiments, the base editor and gRNA are delivered or administered as a protein:RNA complex. In certain embodiments, the method of delivery and vector provided herein is an RNP complex. RNP delivery of base editors markedly increases the DNA specificity of base editing. RNP delivery of base editors leads to decoupling of on- and off-target editing. RNP delivery ablated off-target editing at non-repetitive sites while maintaining on-target editing comparable to plasmid delivery, and greatly reduced off-target editing even at the highly repetitive VEGFA site 2. See Rees, H. A. et al., Improving the DNA specificity and applicability of base editing through protein engineering and protein delivery, Nat. Commun. 8, 15790 (2017), which is incorporated by reference herein in its entirety.

Methods of non-viral delivery of nucleic acids include RNP complexes, lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Lipofectamine, Lipofectamine 2000, Lipofectamine 3000, Transfectam™ and Lipofectin™). In certain embodiments of the disclosed methods of editing and methods of evaluating off-target effects of base editors, a cationic lipid comprising Lipofectamine 2000 is used for delivery of nucleic acids to cells. Cationic and neutral lipids that are suitable for efficient receptor-recognition lipofection of polynucleotides include those of Feigner, WO 1991/17424 and WO 1991/16024. Delivery can be to cells (e.g., in vitro or ex vivo administration) or target tissues (e.g., in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, 4,946,787, 9,526,784, and 9,737,604).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. Reference is made to US 2003-0087817, published May 8, 2003, International Patent Application No. WO 2016/205764, published Dec. 22, 2016, International Patent Application No. WO 2018/071868, published Apr. 19, 2018, U.S. Patent Publication No. 2018/0127780, published May 10, 2018, and International Patent Application No. PCT/US2020/033873, the disclosures of each of which are incorporated herein by reference.

In various embodiments, the disclosed expression constructs may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a whole or split base editors that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAV5 and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US-2007-0015238 and US-2012-0322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

In some embodiments, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning CBE. Reference is made to International Patent Application No. PCT/US2020/033873, incorporated by reference herein.

These split intein-based methods overcome several barriers to in vivo delivery. For example, the DNA encoding base editors is larger than the recombinant AAV (rAAV) packaging limit, and so requires different solutions. One such solution is formulating the editor fused to split intein pairs that are packaged into two separate rAAV particles that, when co-delivered to a cell, reconstitute the functional editor protein. Several other special considerations to account for the unique features of base editing are described, including the optimization of second-site nicking targets and properly packaging base editors into virus vectors, including lentiviruses and rAAV.

Accordingly, the disclosure provides dual rAAV vectors and dual rAAV vector particles that comprise expression constructs that encode two halves of any of the disclosed base editors, wherein the encoded base editor is divided between the two halves at a split site. In some embodiments, the two halves may be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning CBE.

In various embodiments, the base editors may be engineered as two half proteins (i.e., an CBE N-terminal half and a CBE C-terminal half) by “splitting” the whole base editor as a “split site.” The “split site” refers to the location of insertion of split intein sequences (i.e., the N intein and the C intein) between two adjacent amino acid residues in the base editor. More specifically, the “split site” refers to the location of dividing the whole base editor into two separate halves, wherein in each halve is fused at the split site to either the N intein or the C intein motifs. The split site can be at any suitable location in the base editor base editor, but preferably the split site is located at a position that allows for the formation of two half proteins which are appropriately sized for delivery (e.g., by expression vector) and wherein the inteins, which are fused to each half protein at the split site termini, are available to sufficiently interact with one another when one half protein contacts the other half protein inside the cell.

Additional methods for the delivery of nucleic acids to cells are known to those skilled in the art. See, for example, US Publication No. 2003/0087817, incorporated herein by reference.

It should be appreciated that any base editor, e.g., any of the base editors provided herein, may be introduced into the cell in any suitable way, either stably or transiently. In some embodiments, a base editor may be transfected into the cell. In some embodiments, the cell may be transduced or transfected with a nucleic acid construct that encodes a base editor. For example, a cell may be transduced (e.g., with a virus encoding a base editor), or transfected (e.g., with a plasmid encoding a base editor) with a nucleic acid that encodes a base editor, or the translated base editor. Such transduction may be a stable or transient transduction. In some embodiments, cells expressing a base editor or containing a base editor may be transduced or transfected with one or more gRNA molecules, for example when the base editor comprises a Cas9 (e.g., nCas9) domain. In some embodiments, a plasmid expressing a base editor may be introduced into cells through electroporation, transient (e.g., lipofection) and stable genome integration (e.g., piggybac) and viral transduction or other methods known to those of skill in the art.

### Kits and Cells

In some aspects, this disclosure provides kits comprising a nucleic acid construct comprising nucleotide sequences encoding the CBEs, gRNAs, and/or complexes described herein. Some embodiments of this disclosure provide kits comprising a nucleic acid construct comprising a nucleotide sequence encoding a CBE. In some embodiments, the nucleotide sequence comprises a heterologous promoter that drives expression of the base editor. The nucleotide sequence may further comprise a heterologous promoter that drives expression of the gRNA, or a heterologous promoter that drives expression of the base editor and the gRNA.

In some embodiments, the kit further comprises an expression construct encoding a guide nucleic acid backbone, e.g., a guide RNA backbone, wherein the construct comprises a cloning site positioned to allow the cloning of a nucleic acid sequence identical or complementary to a target sequence into the guide nucleic acid, e.g., guide RNA backbone.

The disclosure further provides kits comprising any of the base editors provided herein, a gRNA having complementarity to a target sequence, and one or more of the following: cofactor proteins, buffers, media, and target cells (e.g., human cells).

Some embodiments of this disclosure provide cells comprising any of the base editors or complexes provided herein. In some embodiments, the cells comprise nucleotide constructs that encodes any of the base editors provided herein. In some embodiments, the cells comprise any of the nucleotides or vectors provided herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art.

In other aspects, in the context of the systems and assays provided herein, this disclosure provides kits comprising a nucleic acid construct comprising: (i) a nucleic acid sequence encoding a CBE comprising a Cas9 domain; (ii) a nucleic acid sequence encoding a first gRNA that is engineered to bind to the Cas9 domain of the cytosine base editor, wherein the second guide RNA comprises a sequence that is complementary to a target sequence; (iii) a nucleic acid sequence encoding a first nuclease inactive Cas9 (dCas9) protein; and (iv) a nucleic acid sequence encoding a second gRNA that is engineered to bind to the dCas9 protein, wherein the second guide RNA comprises a sequence that is complementary to an off-target sequence, wherein the off-target sequence has about 60% or less sequence identity to the target sequence. The off-target sequences may otherwise have 6-8 or more mismatches relative to the target sequence. Exemplary kits may further comprise a nucleic acid construct comprising: (v) a nucleic acid sequence encoding a second dCas9 protein; and (vi) a nucleic acid sequence encoding a third gRNA that is engineered to bind to a second dCas9 protein, wherein the third guide RNA is complementary to the third sequence.

The disclosure further provides kits comprising any of the base editors provided herein, a gRNA having complementarity to a target sequence, an isolated dCas9 protein, a second gRNA having complementarity to an off-target sequence, a second isolated dCas9 protein, and a third gRNA having complementarity to the off-target sequence, and one or more of the following: cofactor proteins, buffers, media, and target cells (e.g., human cells). These kits may further comprise complexes containing additional isolated dCas9 proteins and additional gRNAs engineered to bind thereto and having complementarity to an off-target sequence. Kits may comprise combinations of several or all of the aforementioned components.

Some embodiments of this disclosure provide cells comprising any of the base editors or complexes provided herein. In some embodiments, the cells comprise nucleotide constructs that encodes any of the base editors provided herein. In some embodiments, the cells comprise any of the nucleotides or vectors provided herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art.

In some aspects, the present disclosure provides uses of any one of the base editors described herein and a guide RNA targeting this base editor to a target C:G base pair in a nucleic acid molecule in the manufacture of a kit for nucleic acid editing, wherein the nucleic acid editing comprises contacting the nucleic acid molecule with the base editor and guide RNA under conditions suitable for the substitution of the cytosine (C) of the C:G nucleobase pair with an thymine (T). In some embodiments of these uses, the nucleic acid molecule is a double-stranded DNA molecule. In some embodiments, the step of contacting of induces separation of the double-stranded DNA at a target region. In some embodiments, the step of contacting further comprises nicking one strand of the double-stranded DNA, wherein the one strand comprises an unmutated strand that comprises the G of the target C:G nucleobase pair.

In some aspects, the present disclosure provides uses of any one of the base editors described herein and a guide RNA targeting this base editor to a target C:G base pair in a nucleic acid molecule in the manufacture of a kit for evaluating the off-target effects of a base editor, wherein the step of evaluating the off-target effects comprises contacting the base editor with the nucleic acid molecule and determining off-target effects in accordance with any one of the disclosed methods. In some embodiments of these uses, the nucleic acid molecule is a double-stranded DNA molecule. In some embodiments, the step of contacting of induces separation of the double-stranded DNA at a target region. In some embodiments, the step of contacting further comprises nicking one strand of the double-stranded DNA, wherein the one strand comprises an unmutated strand that comprises the G of the target C:G nucleobase pair.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in a cell, such as a human or non-human animal cell.

The present disclosure also provides uses of any one of the base editors described herein as a medicament. The present disclosure also provides uses of any one of the complexes of base editors and guide RNAs described herein as a medicament.

### EXAMPLES

### Example 1

Cas9-independent deamination was assayed by CBEs in bacteria using a rifampin resistance assay. Measuring resistance to the antibiotic rifampin has previously been used to characterize the activity and mutagenicity of proteins expressed in E. coli (15-19). Deaminase-catalyzed C:G-to-T:A mutations in the rpoB gene render E. coli resistant to rifampin. It was hypothesized that cells transformed with a plasmid encoding a base editor with Cas9-independent deamination activity would become resistant to rifampin at a frequency that reflects the magnitude of this activity. To simultaneously assess the on-target activity of the base editor, a second plasmid encoding a defective chloramphenicol acetyltransferase with an inactivating T:A-to-C:G point mutation was transformed, together with a guide RNA that directs the CBE to revert this point mutation. Base editors with higher on-target activity more effectively rescue chloramphenicol resistance (9). This design enables survival rates on chloramphenicol to reflect on-target editing efficiency, and survival rates on rifampin to reflect Cas9-independent deamination activity (FIG. 1A).

To validate this assay, the chloramphenicol and rifampin resistance of bacteria transformed with wild-type APOBEC1, the cytidine deaminase used in BE3, and the catalytically inactive E63A mutant of APOBEC1, were measured. The catalytically inactive E63A mutant of APOBEC1 was measured in three different architectures: as free deaminases, as deaminase-dCas9-UGI fusions, or as deaminase-dCas9 fusions lacking the UGI domain (FIG. 1B). dCas9 was used instead of Cas9 nickase for the prokaryotic cell assays because E. coli lack the nick-directed mismatch repair pathway that enables improved editing by Cas9 nickase CBEs in mammalian cells (20). Compared to the background resistance levels of untethered inactive APOBEC1 E63A construct, untethered active APOBEC1 induced a 1,000-fold increase in rifampin resistance and a 10-fold increase in chloramphenicol resistance. The APOBEC1-dCas9-UGI base editor yielded the same level of rifampin resistance as that of untethered APOBEC1, but a 250-fold higher level of chloramphenicol resistance. These data are consistent with high on-target activity of CBEs, and with Cas9-independent off-target mutagenesis.

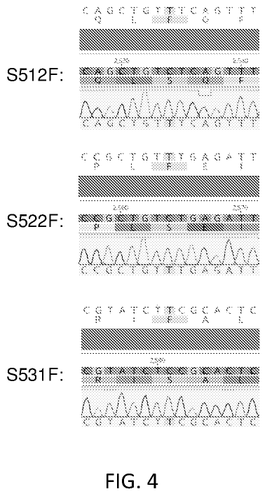

To confirm that rifampin resistance was accompanied by mutations consistent with CBE activity, the rpoB gene of rifampin-resistant colonies was sequenced, and primarily C•G-to-T•A mutations were observed (FIG. 4). The inactive APOBEC1 E63A-dCas9 fusion resulted in rifampin resistance levels equivalent to the background of the assay, suggesting that dCas9 alone does not contribute to these off-target mutations. Compared to the APOBEC1-dCas9-UGI fusion, both the APOBEC1-dCas9 fusion and the APOBEC1 E63A-dCas9-UGI exhibited a substantial decrease in rifampin resistance rates. These results suggest that both the deaminase domain of the base editor as well as the UGI domain can contribute to Cas9-independent off-target mutagenesis.

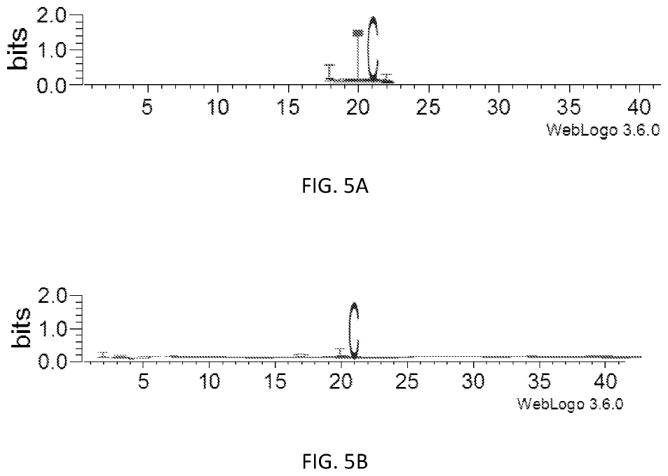

To minimize Cas9-independent deamination, the focus was on the deaminase domain for two reasons. First, the rifampin resistance frequency from expression of APOBEC1 alone was 100-fold higher than the average rifampin resistance from expression of UGI alone (FIG. 1B). Second, when the off-target DNA sequencing data from Zuo et al. (13) was analyzed, a strong 5′ T preference among edited cytosines (FIGS. 5A-5B) was found. This preference suggests that APOBEC1, which has a preference for deaminating 5′ TC substrates (21), is primarily responsible for these off-target edits, as opposed to UGI, which is not known to cause a sequence context bias among C:G-to-T:A mutations.

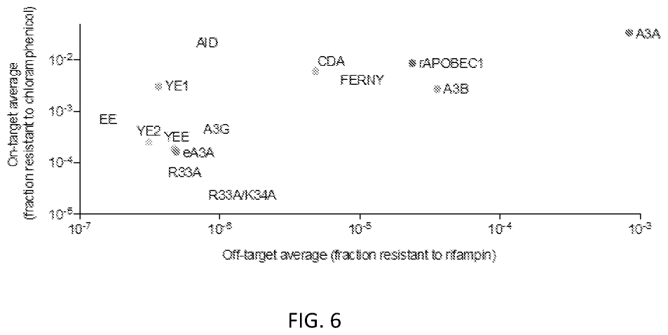

Many alternative deaminase domains among CBE variants following BE3 have now been reported (1, 22-33). The chloramphenicol and rifampin resistance of E. coli transformed with base editors containing virtually all deaminase domains used for cytosine base editing was measured, starting with naturally occurring APOBEC1, AID, CDA, APOBEC3A, APOBEC3B, and APOBEC3G deaminases (FIG. 1C and FIG. 6). E. coli transformed with CBEs that use CDA, APOBEC3A, and APOBEC3B exhibited rifampin resistance levels that were comparable to, or higher than, the rifampin resistance arising from the original APOBEC1 base editor, consistent with the recent characterization of high editing activity from CDA- and APOBEC3A-derived CBEs (30). In contrast, APOBEC3G and AID base editors produced significantly lower levels of rifampin resistance, suggesting they generate less Cas9-independent deamination in bacteria.

Next, the panel of deaminases was expanded to include engineered deaminase variants that had been previously developed for base editing applications. The APOBEC1 variants W90Y+R126E (YE1), W90Y+R132E (YE2), R126E+R132E (EE), and W90Y+R126E+R132E (YEE) were created to narrow the on-target base editing window (31) (Y. B. Kim, et al. Nat. Biotechnol. 35, 371-376 (2017), herein incorporated by reference). In addition, as discussed in Grunewald et al., APOBEC1 variants R33A and R33A+K34A could be engineered to have lower off-target RNA editing (32). Additionally, as discussed in Gehrke et al., APOBEC3A (eA3A) has been engineered to have a strict 5′ T sequence context requirement (33). Promisingly, most of these engineered CBEs yielded substantially lower rifampin resistance levels in bacteria. In particular, eA3A, YE1, YE2, EE, YEE, R33A, and R33A+K34AAPOBEC1 variants all resulted in rifampin resistance frequencies equivalent to that of the inactive APOBEC1 E63A-dCas9-UGI control construct (FIG. 1C and FIG. 6). These results indicate that several recently reported base editor variants have much lower Cas9-independent deamination in E. coli, consistent with their original design goals of lower deamination processivity (31), weaker deamination activity (32), or increased requirements for deamination (33).

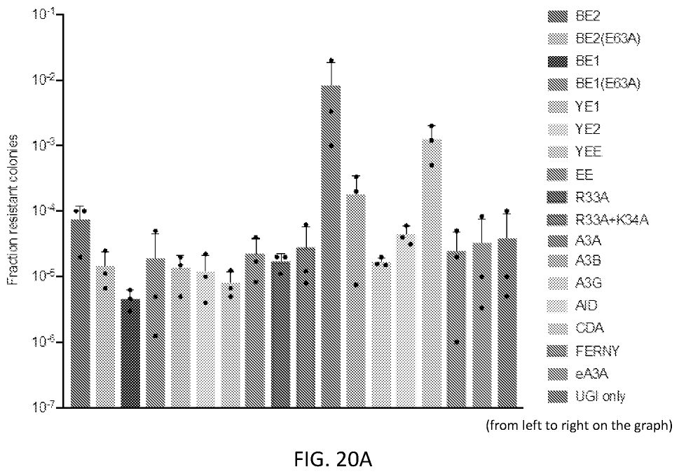

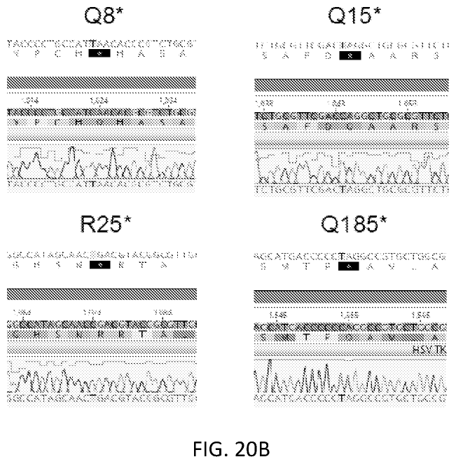

Numerous studies have shown that cytidine deaminases exhibit strong sequence context preferences. For instance, while APOBEC1 prefers 5′-TC substrates, A3G prefers 5′-CC substrates, and AID prefers 5′-GC substrates (23, 24, 29, 30). To ensure that the results of the prokaryotic cell rifampin assay were not skewed by the sequence contexts of particular cytosines whose mutagenesis led to rifampin resistance, it was sought to recapitulate the rifampin assay using a different target gene and selection marker. This new target gene would have a different set of cytosines that yield resistance, and therefore a different set of 5′ bases that could introduce bias among the deaminases. To do this, a single copy of the herpes simplex virus thymidine kinase (HSV-TK) gene was inserted into the E. coli chromosome. HSV-TK leads to toxicity in the presence of the nucleoside analog deoxyribofuranosyl)-3,4-dihydro-8H-pyrimido-[4,5-c][1,2]oxazin-7-one (“dP”) (34). It was reasoned that off-target C:G-to-T:A mutations in the HSV-TK gene that inactivate the enzyme would lead to survival on dP. Indeed, while the dynamic range of this assay was narrower than that of the rifampin assay, the same trends held: rAPOBEC1, A3A, A3B, and CDA induced more mutagenesis, whereas most other CBEs induced levels of dP resistance comparable to background. Sequencing of the HSV-TK gene confirmed various resistant alleles caused by C:G-to-T:A mutations among surviving colonies (FIGS. 20A-20B). The consistency between the rifampin and HSV-TK resistance assays suggests that sequence context bias plays a minimal role in the results.

### Example 2

Next, assays for Cas9-independent deamination by CBEs in human cells that are not dependent on time- and resource-intensive whole-genome sequencing were developed. Since the above results, as well as the findings of Zuo et al. (13, 14), all suggest that the frequency of stochastic Cas9-independent deamination by BE3 is well below the ˜0.1% detection limit of practical high-throughput DNA sequencing experiments, an assay in human cells that magnifies Cas9-independent off-target deamination at specific loci that can be monitored by targeted high-throughput sequencing was sought. All of the deaminases used in CBEs to date are known to deaminate single-stranded DNA or RNA efficiently, but not double-stranded nucleic acids, and recent reports detailing Cas9-independent deamination by BE3 noted that the observed mutations were higher in transcribed regions of the genome (13, 14). Since the low frequency of BE3-induced Cas9-independent deamination is likely the result of the inability of cytidine deaminases to operate on double-stranded DNA, it was reasoned that generating long-lived single-stranded DNA regions at specified positions would enable artificially high Cas9-independent deamination levels sufficient to detect this off-target activity with high sensitivity.

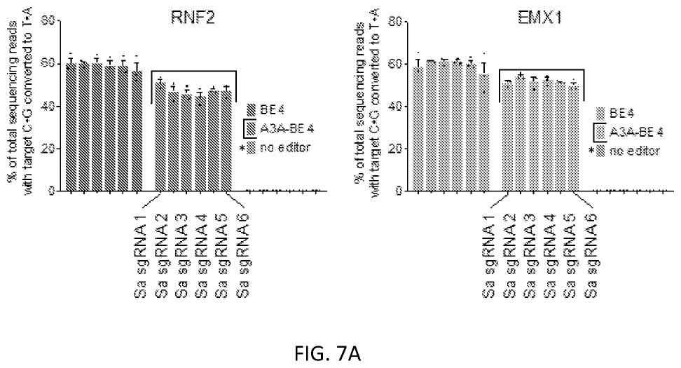

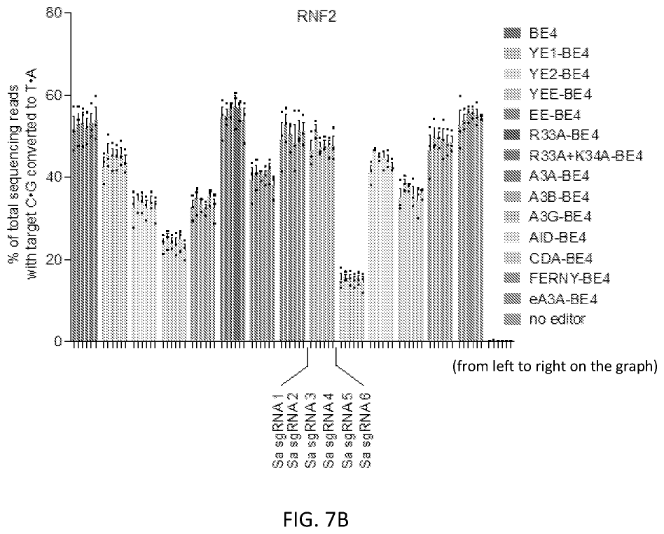

To evaluate the ability of different base editors to deaminate cytosines in single-stranded DNA regions unrelated to their on-target loci, HEK293T cells were co-transfected with plasmids encoding an SpCas9-based CBE, an SpCas9 on-target guide RNA, a catalytically inactive S. aureus Cas9 (dSaCas9), and an SaCas9 guide RNA targeting a genomic locus unrelated to the on-target site (FIGS. 2A-2B). All editors for mammalian cell experiments were generated using the current “BE4max” architecture, with optimized nuclear localization signals, optimized codon usage, and the optimized structure of NLS-deaminase-Cas9 nickase-UGI-UGI-NLS (5, 35). Deamination of cytosines in the R-loop formed by dSaCas9 should occur in a CBE-dependent, but SpCas9 guide RNA-independent, manner. Indeed, high-throughput sequencing of six dSaCas9 loci three days after plasmid co-transfection resulted in off-target deamination levels by APOBEC1-based BE4 (5, 35) that were easily detected by targeted DNA sequencing (0.4-25%), and were independent of the on-target SpCas9 guide RNA (FIG. 2C). Encouragingly, A3A-BE4 (30), a CBE that uses APOBEC3A, demonstrated substantially higher off-target deamination of dSaCas9-generated R-loops relative to BE4 (FIG. 2B and FIG. 7A), consistent with its higher frequency of generating resistant colonies in the prokaryotic rifampin assay (FIG. 1C), and with the previously reported high degree of mutagenicity of APOBEC3A in human cells (36). These results collectively suggest that the level of in trans deamination within R-loops generated by an orthogonal Cas9 homolog can be used to assess the propensities of SpCas9-derived CBEs to mediate Cas9-independent deamination.

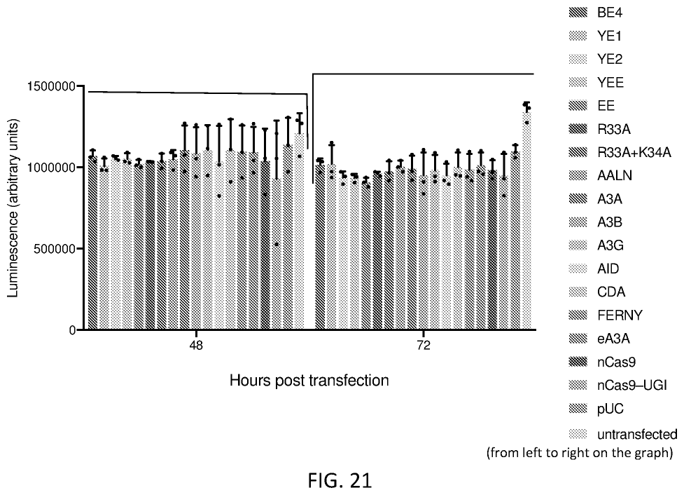

To identify base editor variants that exhibit reduced Cas9-independent deamination relative to BE4 in human cells, the same panel of 14 deaminase domains (APOBEC1, CDA, AID, APOBEC3A, eA3A, APOBEC3B, APOBEC3G, and FERNY, and APOBEC1 mutants YE1, YE2, YEE, EE, R33A, and R33A+K34A) (1, 22-33) were evaluated in the BE4max architecture for their ability to deaminate dSaCas9-induced R-loops in trans. Base editors with narrowed on-target DNA editing windows such as YE1-BE4, YE2-BE4, and EE-BE4, or with reduced RNA editing propensities such as R33A-BE4, again exhibited substantially reduced Cas9-independent DNA deamination compared to BE4 (FIG. 2D and FIG. 7B). Indeed, YEE-BE4 and R33A+K34A-BE4 displayed nearly undetectable levels of Cas9-independent deamination in this assay. Nearly all of the other CBE variants assayed displayed comparable or higher levels of Cas9-independent deamination relative to BE4 for at least a subset of off-target cytosines within SaCas9-induced R-loops. Compared to BE4, CBEs derived from CDA, AID, and FERNY exhibited higher levels of Cas9-independent deamination at 5′-GC substrates, as expected given their higher activity on 5′-GC sequences than APOBEC1 (25, 26, 32), but not generally at 5′-TC substrates. Likewise, eA3A-BE4 and A3G-BE4 displayed moderate to high levels of Cas9-independent deamination at 5′-TCR and 5′-CC substrates respectively, also consistent with their known sequence context preferences (29,33). All transfected constructs had similar effects on cell viability (FIG. 21), which indicated that cell viability was not a confounding factor in this assay. Importantly, the trends observed in this assay agree with the results of the rifampin resistance and thymidine kinase assays, with the exception of AID-BE4: the results show higher amounts of off-target editing by AID-BE4 in mammalian cells compared to in E. coli. This observation is consistent with previous studies that show higher AID activity in human cells compared to bacteria, potentially due to protein/protein interaction partners or post-translational modifications to the enzyme (17, 37). These data suggest that YE1-BE4, YE2-BE4, EE-BE4, YEE-BE4, and R33A+K34A-BE4 are especially promising CBE variants for applications in which Cas9-independent off-target editing must be minimized.

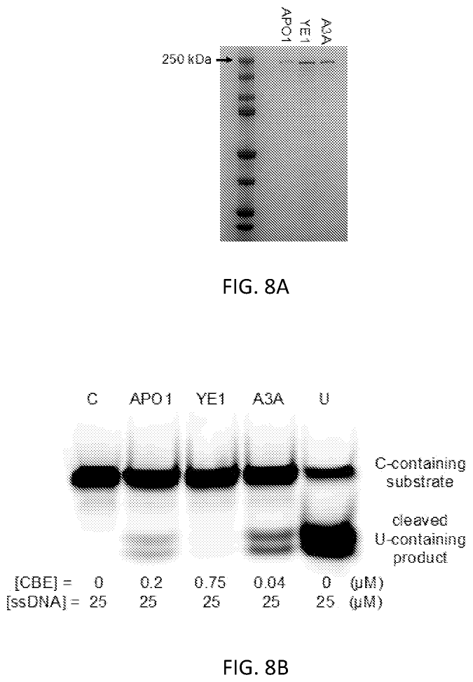

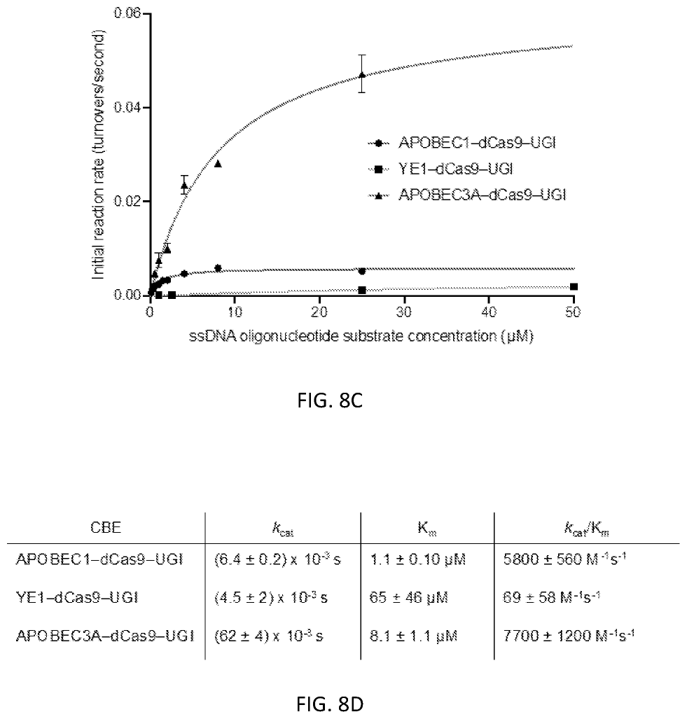

It was hypothesized that a primary determinant of Cas9-independent deamination propensity is the catalytic efficiency of the enzyme. Deaminases for base editing should inefficiently catalyze deamination of substrates that are present at low concentrations (such as Cas9-independent off-target sites) but efficiently deaminate on-target substrates when presented at high effective local concentration due to DNA binding of the tethered Cas9 domain. To test this, three different CBE proteins were purified, and their k_{cat}/Kₘ values in vitro were measured for a 5′-Cy3-labeled ssDNA oligonucleotide that contained a single cytosine and was unrelated to the sgRNA present in the reaction. To measure reaction velocities, uracil-containing product formation by gel densitometry following USER enzyme treatment (38) was quantified. YE1-dCas9-UGI and APOBEC3A-dCas9-UGI were observed to have k_{cat}/Kₘ values for ssDNA that are 69-fold lower and 1.3-fold higher, respectively, than that of APOBEC1-dCas9-UGI (FIGS. 8A-8D). These results are consistent with the orthogonal R-loop assays in FIGS. 2A-2D, as well as the rifampin resistance assays in FIGS. 1A-1C, and support a model in which CBEs with higher k_{cat}/Kₘ values for ssDNA have a greater propensity for Cas9-independent deamination in cells.

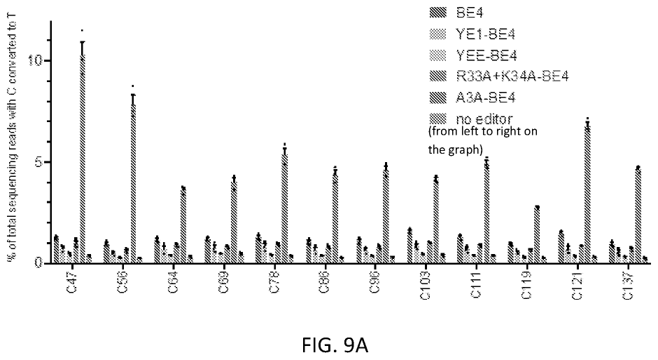

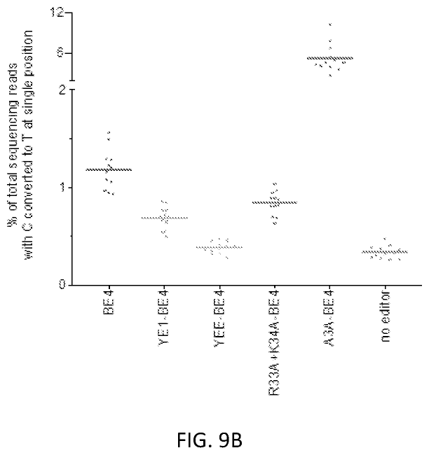

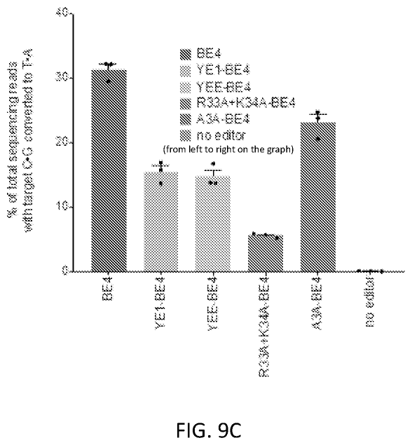

As an additional independent assay of Cas9-independent deamination by CBEs in mammalian cells, intracellular deamination frequencies were also measured from BE4, A3A-BE4, YE1-BE4, YEE-BE4, and R33A+K34A-BE4 of a co-transfected 164-mer ssDNA oligonucleotide containing 35 cytosines in HEK293T cells, in light of previous reports that endogenous deaminases can induce mutagenesis in transfected ssDNA oligonucleotides (FIGS. 9A-9C) (39). A3A-BE4 showed 4.4-fold higher Cas9-independent off-target editing compared to BE4, while YE1-BE4, YEE-BE4, and R33A+K34A-BE4 showed 1.7-, 3.2-, and 1.4-fold lower average Cas9-independent off-target editing relative to BE4 at the twelve 5′-TC cytosines present in the oligonucleotide that were deaminated above background (FIGS. 9A-9C), again concordant with findings from the other assays. Taken together, the results from the rifampin and HSV-TK resistance assays in bacteria, orthogonal R-loop assay in human cells, kinetic assay in vitro, and ssDNA deamination assay in human cells are consistent with a model in which CBEs with deaminases that have a low intrinsic catalytic efficiency (k_{cat}/Kₘ) for cytosine-containing ssDNA substrates exhibit lower Cas9-independent off-target deamination.

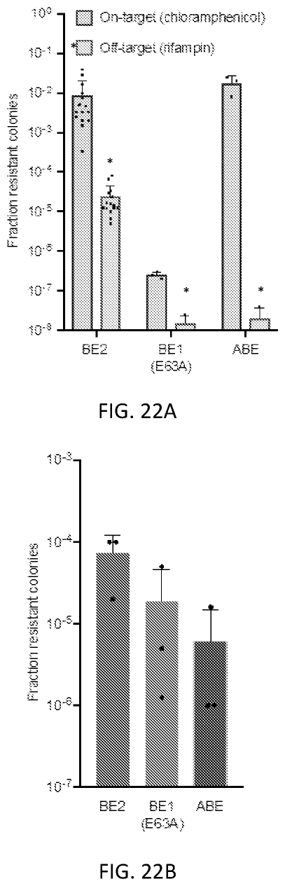

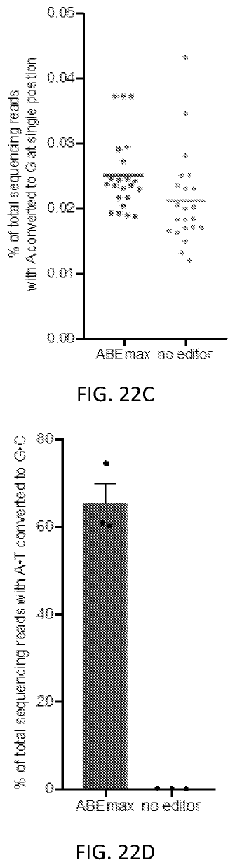

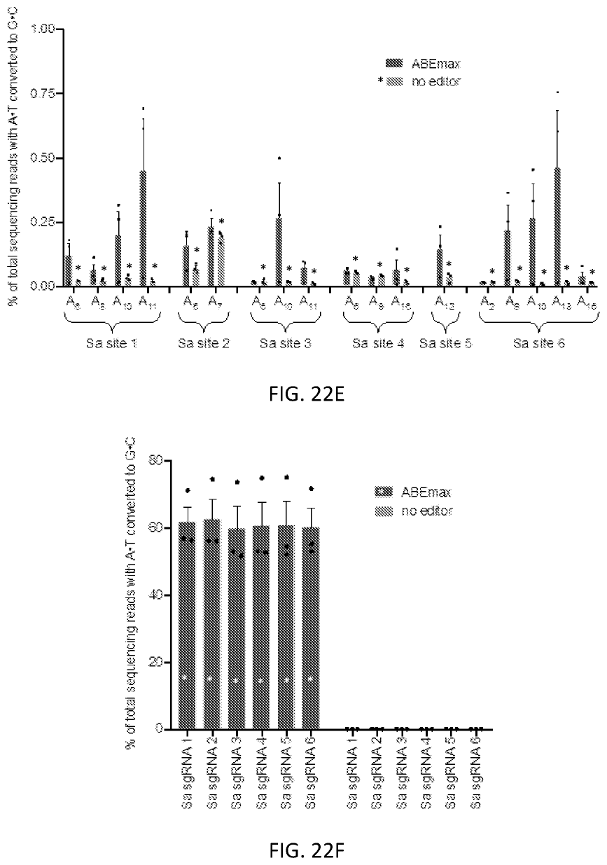

The previous studies that detected Cas9-independent off-target DNA editing by CBEs notably did not detect any such off-target editing induced by the canonical adenine base editor, ABE (13, 14). Therefore, it was suspected that ABE would exhibit minimal Cas9-independent off-target editing in the assays described above. Indeed, in the rifampin and HSV-TK resistance assays, ABE induced background levels of resistance, and in the orthogonal R-loop and intracellular ssDNA deamination assays, ABE induced only very low levels of off-target A•T-to-G•C editing (FIGS. 22A-22F). These results highlight the consistency between low off-target activity as assessed by the methods developed herein, and low off-target activity as assessed by previous whole-genome sequencing studies (13, 14).

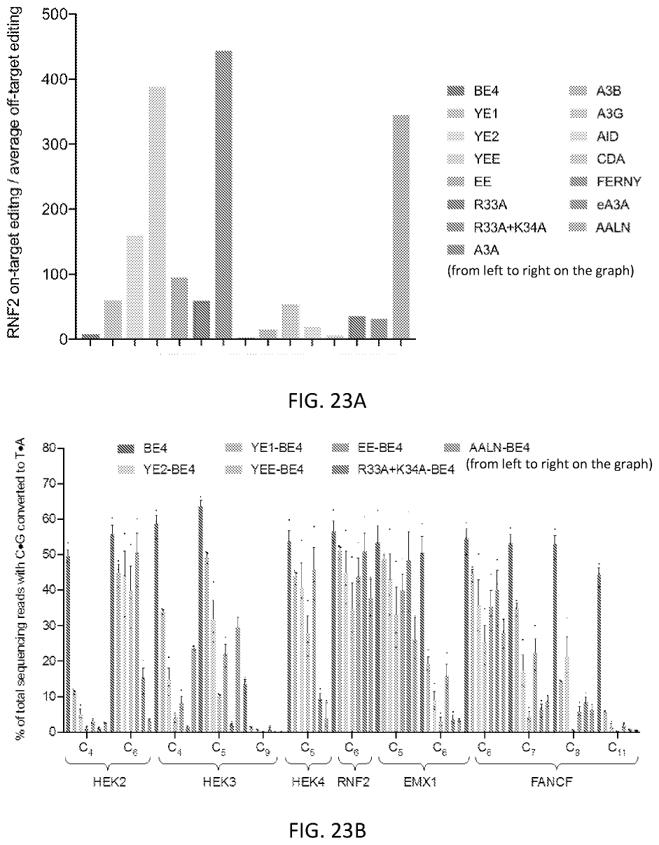

Each deaminase domain tested had a distinct on-target editing and off-target editing profile, as shown in FIG. 3A. Of the CBEs that were identified as being especially promising for minimizing Cas9-independent editing, YE1-BE4 and R33A-BE4 offered the best balance between decreased off-target editing and robust on-target activity (FIG. 3B and FIGS. 23A-23B). Meanwhile, YE2-BE4, EE-BE4, R33A+K34A-BE4, and YEE-BE4 produced even lower off-target editing but with a significant decrease in average on-target activity tested across six sites (FIG. 3B and FIGS. 23A-23B).

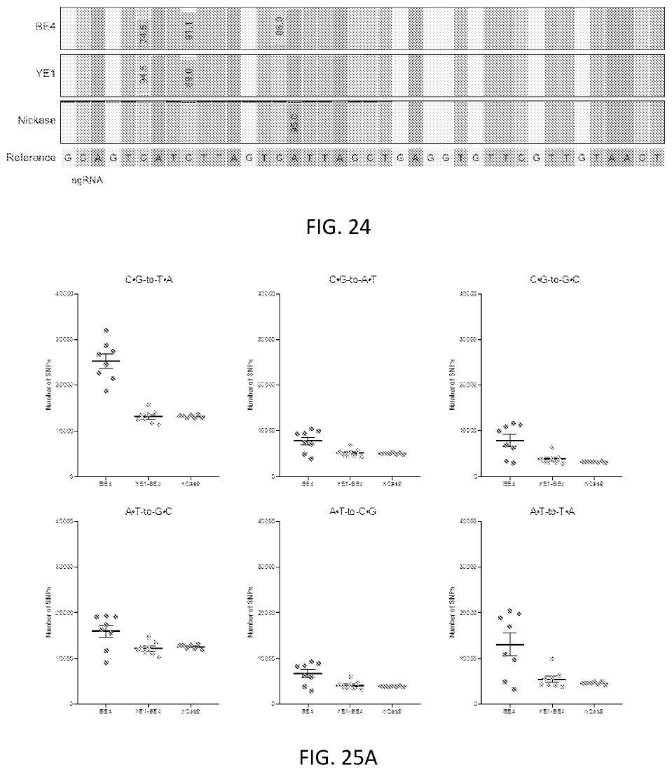

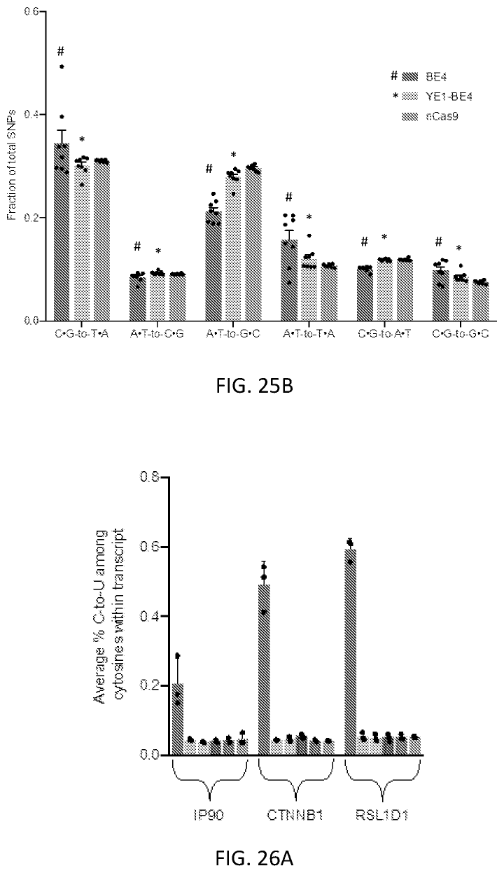

To further validate that the above methods for detecting Cas9-independent off-target DNA editing are representative of genome-wide Cas9-independent off-targets, whole-genome sequencing (WGS) of HEK293T cells treated with BE4, YE1-BE4, or a Cas9 D10A nickase control, was performed. Four days following transfection with an sgRNA plasmid and a plasmid encoding either BE4, YE1-BE4, or nCas9(D10A) cotranslationally fused to GFP, the top ˜25% of GFP-positive cells were isolated by flow cytometry, single cells were diluted into individual wells, and grown into clonal populations for 16 days before genomic DNA extraction. This process ensured that any off-target mutations caused by CBE treatment would be present at high allele frequencies within the clonal population of cells derived from a single CBE-treated cell (FIG. 24). WGS was performed at an average depth of 77× on all samples and it was determined that all single-nucleotide variants (SNVs) were present in each sample using the intersection of variants called by three algorithms (FIG. 24, Table 6, Table 7). In order to restrict the analysis to SNVs that were generated following CBE treatment, the SNVs that were present in the original clonal population of cells prior to CBE treatment were filtered out. The WGS results revealed that BE4, but not YE1, produced significantly more C•G-to-T•A SNVs than the nickase-only negative control (FIG. 3C). This confirmed the findings of Yang et al. and Gao et al. that CBEs containing wild type rAPOBEC1 produce off-target C•G-to-T•A SNVs in a Cas9-independent manner. It was also found that BE4-treated samples contained more non-C:G-to-T:A SNVs than YE1 or nickase samples (FIG. 3D and FIGS. 25A-25B), consistent with previous reports that deaminase overexpression in HEK293 cells leads to overall increased SNVs of all types (40). The frequency of BE4-mediated off-target edits that were observed (1.4×10⁻⁶/bp) was also much higher than either of the previously reported values (5×10⁻⁸/bp and 1.7×10⁻⁷/bp reported by Yang and Gao, respectively). This difference likely arises from the different delivery methods used, the sorting of cells to isolate those that express CBEs most highly, and the different cell types used. Importantly, the above WGS results also confirmed the findings of other assays: YE1 exhibits significantly reduced Cas9-independent off-target editing compared to BE4; indeed, YE1 treatment did not lead to statistically significant differences relative to the Cas9 nickase-only control (FIG. 3D and FIGS. 25A-25B).

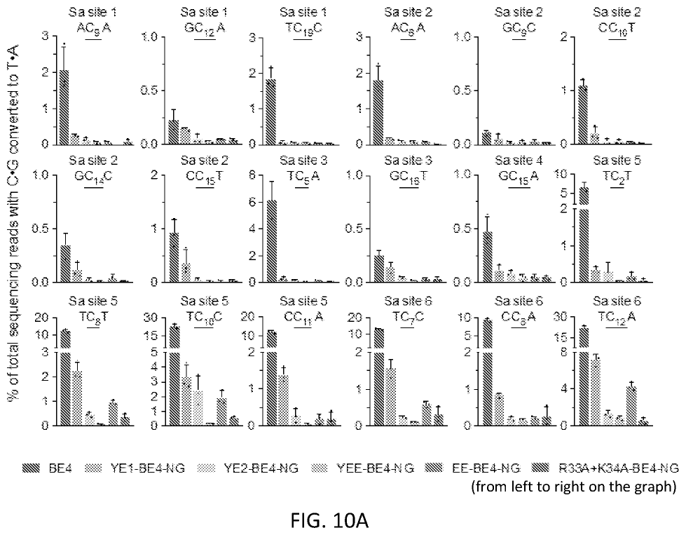

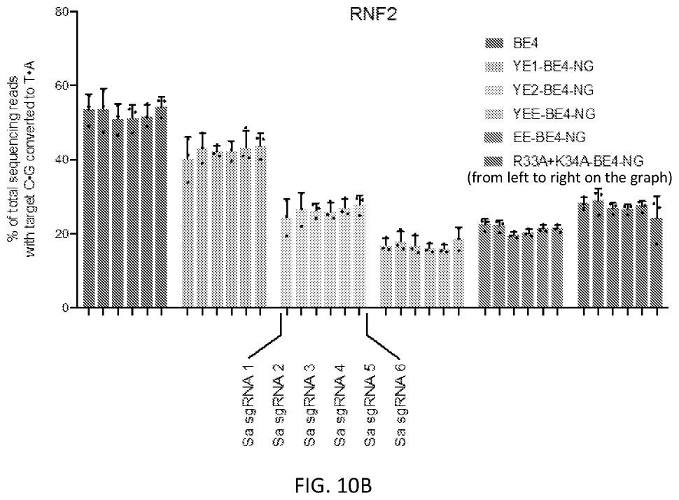

As the assessed CBE variants and YE1 have minimal Cas9-independent off-target activity, all exhibit narrowed on-target DNA editing windows (31) (YE1-BE4, YE2-BE4, YEE-BE4, and EE-BE4), or a specific DNA sequence context requirement (32) (R33A+K34A-BE4), an expansion of the targeting scope of these CBEs in order to increase their overall utility was sought. These deaminase variants were tested for their compatibility with SpCas9-NG, one of two recently reported Cas9 variants that recognize a broadened NG PAM (41,42) and found that YE1, and to a lesser extent YE2, YEE, EE, and R33A+K34A, maintained compatibility with SpCas9-NG nickase (FIG. 4A). YE1-NG expands the targeting scope of CBEs while maintaining minimal substantially decreased Cas9-independent off-target activity (FIGS. 10A-10B).

TABLE 4
Target protospacers and amplicons along with corresponding primers used for genomic
DNA amplification.
SpCas9 genomic loci: Top to bottom, left to right the sequences correspond to SEQ ID NOs: 16-45

| Site name | Protospacer | PAM | Amplicon | HTS_fwd | HTS_rev |
|---|---|---|---|---|---|
| HEK2 | GAACACAAAGCATAGACTGC | GGG | TGAATGGATTCCTTGGAAACAATGATAACAAGACCTGGCTGAGCTAAC | ACACTCT | TGGAGTT |
| | | | TGTGACAGCATGTGGTAATTTTCCAGCCCGCTGGCCCTGTAAAGGAAA | TTCCCTA | CAGACGT |
| | | | CTGGAACACAAAGCATAGACTGCGGGGCGGGCCAGCCTGAATAGCTG | CACGACG | GTGCTCT |
| | | | CAAACAAGTGCAGAATATCTGATGATGTCATACGCACAGTTTGACAGAT | CTCTTCC | TCCGATC |
| | | | GGGGCTGG | GATCTNN | TTGAATG |
| | | | | NNCCAGC | GATTCCT |
| | | | | CCCATCT | TGGAAAC |
| | | | | GTCAAAC | AATGA |
| | | | | T | |
| HEK3 | GGCCCAGACTGAGCACGTGA | TGG | ATGTGGGCTGCCTAGAAAGGCATGGATGAGAGAAGCCTGGAGACAGG | ACACTCT | TGGAGTT |
| | | | GATCCCAGGGAAACGCCCATGCAATTAGTCTATTTCTGCTGCAAGTAA | TTCCCTA | CAGACGT |
| | | | GCATGCATTTGTAGGCTTGATGCTTTTTTTCTGCTTCTCCAGCCCTGGC | CACGACG | GTGCTCT |
| | | | CTGGGTCAATCCTTGGGGCCCAGACTGAGCACGTGATGGCAGAGGA | CTCTTCC | TCCGATC |
| | | | AAGGAAGCCCTGCTTCCTCCAGAGGGCGTCGCAGGACAGCTTTTCCT | GATCTNN | TCCCAGC |
| | | | AGACAGGGGCTAGTATGTGCAGCTCCTGCACCGGGATACTGGTTGAC | NNATGTG | CAAACTT |
| | | | AAGTTTGGCTGGG | GGCTGCC | GTCAACC |
| | | | | TAGAAAG | |
| | | | | G | |
| HEK4 | GGCACTGCGGCTGGAGGTGG | GGG | GAACCCAGGTAGCCAGAGACCCGCTGGTCTTCTTTCCCCTCCCCTGC | ACACTCT | TGGAGTT |
| | | | CCTCCCCTCCCTTCAAGATGGCTGACAAAGGCCGGGCTGGGTGGAA | TTCCCTA | CAGACGT |
| | | | GGAAGGGAGGAAGGGCGAGGCAGAGGGTCCAAAGCAGGATGACAG | CACGACG | GTGCTCT |
| | | | GCAGGGGCACCGCGGCGCCCCGGTGGCACTGCGGCTGGAGGTGGG | CTCTTCC | TCCGATC |
| | | | GGTTAAAGCGGAGACTCTGGTGCTGTGTGACTACAGTGGGGGCCCTG | GATCTNN | TTCCTTT |
| | | | CCCTCTCTGAGCCCCCGCCTCCAGGCCTGTGTGTGTGTCTCCGTTCG | NNGAACC | CAACCCG |
| | | | GGTTGAAAGGA | CAGGTAG | AACGGAG |
| | | | | CCAGAGA | |
| | | | | C | |
| RNF2 | GTCATCTTAGTCATTACCTG | AGG | ACGTCTCATATGCCCCTTGGCAGTCATCTTAGTCATTACCTGAGGTGTT | ACACTCT | TGGAGTT |
| | | | CGTTGTAACTCATATAAACTGAGTTCCCATGTTTTGCTTAATGGTTGAGT | TTCCCTA | CAGACGT |
| | | | TCCGTTTGTCTGCACAGCCTGAGACATTGCTGGAAATAAAGAAGAGAG | CACGACG | GTGCTCT |
| | | | AAAAACAATTTTAGTATTTGGAAGGGAAGTGCTATGGTCTGAATGTATG | CTCTTCC | TCCGATC |
| | | | TGTCCCACCAAAATTCCTACGT | GATCTNN | TACGTAG |
| | | | | NNACGTC | GAATTTT |
| | | | | TCATATG | GGTGGG |
| | | | | CCCCTTG | ACA |
| | | | | G | |
| EMX1 | GAGTCCGAGCAGAAGAAGAA | GGG | CAGCTCAGCCTGAGTGTTGAGGCCCCAGTGGCTGCTCTGGGGGCCT | ACACTCT | TGGAGTT |
| | | | CCTGAGTTTCTCATCTGTGCCCCTCCCTCCCTGGCCCAGGTGAAGGT | TTCCCTA | CAGACGT |
| | | | GTGGTTCCAGAACCGGAGGACAAAGTACAAACGGCAGAAGCTGGAG | CACGACG | GTGCTCT |
| | | | GAGGAAGGGCCTGAGTCCGAGCAGAAGAAGAAGGGCTCCCATCACA | CTCTTCC | TCCGATC |
| | | | TCAACCGGTGGCGCATTGCCACGAAGCAGGCCAATGGGGAGGACAT | GATCTNN | TCTCGTG |
| | | | CGATGTCACCTCCAATGACTAGGGTGGGCAACCACAAACCCACGAG | NNCAGCT | GGTTTGT |
| | | | | CAGCCTG | GGTTGC |
| | | | | AGTGTTG | |
| | | | | A | |
| FANCF | GGAATCCCTTCTGCAGCACC | TGG | CATTGCAGAGAGGCGTATCATTTCGCGGATGTTCCAATCAGTACGCAG | ACACTCT | TGGAGTT |
| | | | AGAGTCGCCGTCTCCAAGGTGAAAGCGGAAGTAGGGCCTTCGCGCA | TTCCCTA | CAGACGT |
| | | | CCTCATGGAATCCCTTCTGCAGCACCTGGATCGCTTTTCCGAGCTTCT | CACGACG | GTGCTCT |
| | | | GGCGGTCTCAAGCACTACCTACGTCAGCACCTGGGACCCC | CTCTTCC | TCCGATC |
| | | | | GATCTNN | TGGGGTC |
| | | | | NNCATTG | CCAGGTG |
| | | | | CAGAGAG | CTGAC |
| | | | | GCGTATC | |
| | | | | A | |

SaCas9 genomic loci: Top to bottom, left to right the sequenes correspond to SEQ ID NOs: 46-76

| Site name | Protospacer | PAM | Amplicon | HTS_fwd | HTS_rev |
|---|---|---|---|---|---|
| Sa site 1 | GTGGTAGACAGCATGTGTCCTA | AAGGGT | TGGTGGAGTGCTCTGTGTTTGTCTTTATAA | ACACTCTT | TGGAGTTC |
| | | | ACCCAGATGAGAGGATGAAGGCAACAAGC | TCCCTACA | AGACGTG |
| | | | TTCTGTACCAACATACATGCCCCTTTGCCT | CGACGCT | TGCTCTTC |
| | | | CAAGTCTGGTTATTTTAGGGGGATGCTAGG | CTTCCGAT | CGATTGGT |
| | | | TTGCTTTGGGTCTACCTTACTGAGAAAATG | CTNNNNT | GGAGTGC |
| | | | GCCCCAGGTCATTGTCATGTCCAGTTGTG | GCAGTCT | TCTGTGTT |
| | | | GTAGACAGCATGTGTCCTAAAGGGTATATT | CCTGCTTC | TG |
| | | | CACATGCATGTGCAAAAATACAGGGGTCCT | TCTG | |
| | | | TCTAACCCTATCACAGAGAAGCAGGAGACT | | |
| | | | GC | | |
| Sa site 2 | ATTTACAGCCTGGCCTTTGGGG | TCGGGT | GCTACAGAAAGGTCAGCAGCTATATTTAAC | ACACTCTT | TGGAGTTC |
| | | | CTCAGACCAGGGTGCGGTGGGAGATCTG | TCCCTACA | AGACGTG |
| | | | GTTTCCGGAAGACGGAATGGGGAGAAGG | CGACGCT | TGCTCTTC |
| | | | GCAGGTTCCCCGAGGCGCCCAGACACCC | CTTCCGAT | CGATGCTA |
| | | | AATCCTCCCGGTGACATTTACAGCCTGGC | CTNNNNG | CAGAAAG |
| | | | CTTTGGGGTCGGGTCAACGCTAGGCTGGC | GACATTTC | GTCAGCA |
| | | | AGGGGAAGGGCGGGGCCGTGAGGTGAG | CACCGCA | GC |
| | | | CCGGCGCTGCAGGAAGGGGCCACCACCA | AAATG | |
| | | | GAGGGGCCATTTTGCGGTGGAAATGTCC | | |
| Sa site 3 | GTGTCAGGTAATGTGCTAAACA | GAGAGT | GCTGTGGCATCCAGAGACATGGTTTCTTATCT | ACACTCTT | TGGAGTT |
| | | | CCTTAAGTGTTCAGCTGCTTTTCTTTCATTTATT | TCCCTACA | CAGACGT |
| | | | CCACATATAATTACTATAATTGCTAAACATTTATT | CGACGCT | GTGCTCT |
| | | | TAGTGTCAGGTAATGTGCTAAACAGAGAGTTAC | CTTCCGAT | TCCGATC |
| | | | TGCTCAGACATGTAATAATAATAAATAACACATC | CTNNNNCT | TGCTGTG |
| | | | AAATAACCATACCATTTTAAGCTGTAGTATTATG | GCACCTA | GCATCCA |
| | | | AAGGGAAATCTGGAGCAAAGAGAATAGACTGT | GCCTCCAT | GAGACAT |
| | | | AGGGAAACCAGTTAAGAAATAGGACATGGAGG | GTC | |
| | | | CTAGGTGCAG | | |
| Sa site 4 | GGTGGAGGAGGGTGCATGGGGT | CAGAAT | GGAGGTGGAGAGAGGATGTTTTGCTTATC | ACACTCT | TGGAGTT |
| | | | CAGAAAAGGGAGTGATTGCTTCCAGGGGC | TTCCCTA | CAGACGT |
| | | | CTCAGGGGAATAAATCATAGAATCCTGGAC | CACGACG | GTGCTCT |
| | | | AAGGTTTGAAGGACAGGTAGGATTTGGGT | CTCTTCC | TCCGATC |
| | | | GGGTGGAGGAGGGTGCATGGGGTCAGAA | GATCTNN | TTCCTGA |
| | | | TTGTAACCGAAAACTCATTCCAGGTGGATA | NNGGAG | GGTCTAG |
| | | | GAGAAAATTTCTAGTGTTGTTGTTTTTAAAC | GTGGAGA | GAACCCG |
| | | | TATTTGGGGGACTGGCACAGACCCTTTTTG | GAGGATG | |
| | | | AATACCTGATGGGCTCACATTTCTGTCGAA | T | |
| | | | TCCCAGCGGGTTCCTAGACCTCAGGA | | |
| Sa site 5 | TCTGCTTCTCCAGCCCTGGC | CTGGGT | ATGTGGGCTGCCTAGAAAGGCATGGATGA | ACACTCT | TGGAGTT |
| | | | GAGAAGCCTGGAGACAGGGATCCCAGGG | TTCCCTA | CAGACGT |
| | | | AAACGCCCATGCAATTAGTCTATTTCTGCT | CACGACG | GTGCTCT |
| | | | GCAAGTAAGCATGCATTTGTAGGCTTGATG | CTCTTCC | TCCGATC |
| | | | CTTTTTTTCTGCTTCTCCAGCCCTGGCCTG | GATCTNN | TCCCAGC |
| | | | GGTCAATCCTTGGGGCCCAGACTGAGCAC | NNATGTG | CAAACTT |
| | | | GTGATGGCAGAGGAAAGGAAGCCCTGCTT | GGCTGCC | GTCAACC |
| | | | CCTCCAGAGGGCGTCGCAGGACAGCTTTT | TAGAAAG | |
| | | | CCTAGACAGGGGCTAGTATGTGCAGGTCC | G | |
| | | | TGCACCGGGATACTGGTTGACAAGTTTGG | | |
| | | | CTGGG | | |
| Sa site 6 | GATGTTCCAATCAGTACGCA | GAGAGT | CATTGCAGAGAGGCGTATCATTTCGCGGAT | ACACTCT | TGGAGTT |
| | | | GTTCCAATCAGTACGCAGAGAGTCGCCGT | TTCCCTA | CAGACGT |
| | | | CTCCAAGGTGAAAGCGGAAGTAGGGCCTT | CACGACG | GTGCTCT |
| | | | CGCGCACCTCATGGAATCCCTTCTGCAGC | CTCTTCC | TCCGATC |
| | | | ACCTGGATCGCTTTTCCGAGCTTCTGGCG | GATCTNN | TGGGGTC |
| | | | GTCTCAAGCACTACCTACGTCAGCACCTG | NNCATTG | CCAGGTG |
| | | | GGACCCC | CAGAGAG | CTGAC |
| | | | | GCGTATC | |

SpCas9-NG genomic loci: Top to bottom, left to right the sequenes correspond to SEQ ID NOs: 77-92.

| Name | Protospacer | PAM | Amplicon | HTS_fwd | HTS_rev |
|---|---|---|---|---|---|
| NG site 1 | CAGTCATCTTAGTCATTACC | TGA | ACGTCTCATATGCCCCTTGGCAGTCATCTT | ACACTCT | TGGAGTT |
| | | | AGTCATTACCTGAGGTGTTCGTTGTAACT | TTCCCTA | CAGACGT |
| | | | CATATAAACTGAGTTCCCATGTTTTGCTTA | CACGACG | GTGCTCT |
| | | | ATGGTTGAGTTCCGTTTGTCTGCACAGCC | CTCTTCC | TCCGATC |
| | | | TGAGACATTGCTGGAAATAAAGAAGAGAG | GATCTNN | TACGTAG |
| | | | AAAAACAATTTTAGTATTTGGAAGGGAAGT | NNACGTC | GAATTTT |
| | | | GCTATGGTCTGAATGTATGTGTCCCACCA | TCATATG | GGTGGG |
| | | | AAATTCCTACGT | CCCCTTG | ACA |
| NG site 2 | GCCAGTCCGCGCTCTACTCA | CGC | CCAGAGAGAAAGGAGAGGGAGCGGCGA | ACACTCT | TGGAGTT |
| | | | GCCCTCGCCTCTGCAGCTAGGAGAGGCC | TTCCCTA | CAGACGT |
| | | | AGAGGCAGGCGAGGGAGAGGGTGCGGC | CACGACG | GTGCTCT |
| | | | GATCGCAGGCGAACTCCGCCGCGGGCC | CTCTTCC | TCCGATC |
| | | | GACGCTGGGAGACGACCAGCCCGTAGCC | GATCTNN | TAGTCCA |
| | | | TCCGCCCAACACGGCGGGCGCGCCCGG | NNGACCC | GAGAGAA |
| | | | GAGTCTCCGGCACCCACCCGGTCCCCAT | GATGCGG | AGGAGAG |
| | | | TCCCACCGCACCCGCTACCGGCCAGTCC | TTAGAG | GGAG |
| | | | GCGCTCTACTCACGCTCGGGGCTCTTCC | | |
| | | | AGGCTCCGCGGCTCTAACCGCATCGGGT | | |
| | | | C | | |
| NG site 3 | GATGACCCGTATTATCTGGC | AGT | TTCTAGATGCCGACAAAAGGATCAAGGTG | ACACTCT | TGGAGTT |
| | | | GCGAAGCCCGTGGTGGAGATGGATGGTG | TTCCCTA | CAGACGT |
| | | | ATGAGATGACCCGTATTATCTGGCAGTTCA | CACGACG | GTGCTCT |
| | | | TCAAGGAGAAGGTAGTGCCCCCTCCTGA | CTCTTCC | TCCGATC |
| | | | AGTGGGTGGCTCTCCAGGTGGGCTGGCC | GATCTNN | TAGTGAC |
| | | | AGGGATTGTTCTGTCCCACAGGGTCTTCT | NNTTCTA | AGAGGGA |
| | | | GGACTGCAGGTCCCTAGGACCCCCCCGT | GATGCCG | GAGAAAC |
| | | | TGTCCTGGTAGGGAGGAGCAGCTCTGTTT | ACAAAAG | AGAGC |
| | | | CTCTCCCTCTGTC | GAT | |
| NG site 4 | GGAACAAGGTACTCTTTGAG | TGT | GTAGAAATGGGGTCTTGCTTTGTTGCCCA | ACACTCT | TGGAGTT |
| | | | GGCTGGTCTAAAAAAATATACTACTTTTAT | TTCCCTA | CAGACGT |
| | | | GGATCATACTGCTAAACACTAATATAACCT | CACGACG | GTGCTCT |
| | | | TTGGAAATATAAATCTATATACTTCCTTACC | CTCTTCC | TCCGATC |
| | | | TGGGATTGGAACAAGGTACTCTTTGAGTG | GATCTNN | TAGTGTA |
| | | | TTCACATTGTCACATAAGGGTTCTCCTCCA | NNTTGAG | GAAATGG |
| | | | TGGTAGATACCTGTTCGAACATAGATCTAA | TCTATCG | GGTCTTG |
| | | | AAGAAAAAAGTAGGTATATACTAATGTATAC | AGTGTGT | CTTTG |
| | | | ACTCAACATACACATATGCACACACTCGAT | GCAT | |
| | | | AGACTCAA | | |
| NG site 5 | GCCACAGTCTGGATGGCGGT | TGT | ATCTCTTCAGCCCCTGAGTTGTCACTGGG | ACACTCT | TGGAGTT |
| | | | TGAGCCCGACCGGAAGTCCATCTCCTCC | TTCCCTA | CAGACGT |
| | | | TCCTCCTGCTTCTTGAGGCCGTCAGCCA | CACGACG | GTGCTCT |
| | | | CAGTCTGGATGGCGGTTGTCCACTCCTC | CTCTTCC | TCCGATC |
| | | | CCTGCAGGAGGTCAGGTGAGGCTGCAG | GATCTNN | TAGTATCT |
| | | | GCCTGTACCAGATCAGGAGCTCCACCCC | NNTTCGT | CTTCAGC |
| | | | ACGTCTTTCACCCACCCGCCAGCCTCAC | GACCCTG | CCCTGAG |
| | | | AAGCGTGGTTOCACAGCTGTCGGGGTTC | AGTGTAT | TTGT |
| | | | CCAGAGACAGCCCTGAGGAGGCCACCAC | GTG | |
| | | | TTGACCTGGTCTGGCCACATACACTCAGG | | |
| | | | GTCACGAA | | |

SpCas9 GUIDE-seq off-target sites: Top to bottom, left to right the sequenes correspond to SEQ ID
NOs: 93-203.

| Name | Protospacer | PAM | Amplicon | HTS_fwd | HTS_rev |
|---|---|---|---|---|---|
| HEK3_off1 | CACCCAGACTGAGCACGTGC | TGG | TCCCCTGTTGACCTGGAGAAGCATGAAC | ACACTCTTTCCCTACAC | TGGAGTTCAGACGTG |
| | | | CAGTCAAAAAGTTTAAAGACAAGAGCATT | GACGCTCTTCCGATCTN | TGCTCTTCCGATCTCA |
| | | | AACTGCACCAGTGGGCAGCTCAGCTCA | NNNTCCCCTGTTGACC | CTGTACTTGCCCTGA |
| | | | GACACCAGTAGCGTGGGCACCCAGACT | TGGAGAA | CCA |
| | | | GAGCACGTGCTGGAGCCCAAGAAATGC | | |
| | | | AGAGACCTGTGCACCTCTGGTCAGGGC | | |
| | | | AAGTACAGTG | | |
| HEK3_off2 | GACACAGACTGGGCACGTGA | GGG | TTGGTGTTGACAGGGAGCAACTTCACAG | ACACTCTTTCCCTACAC | TGGAGTTCAGACGTG |
| | | | TCCCAGGCATCAGGACACAGACTGGGC | GACGCTCTTCCGATCTN | TGCTCTTCCGATCTCT |
| | | | ACGTGAGGGAAGCCCAAGGGAGAGGAC | NNNTTGGTGTTGACAG | GAGATGTGGGCAGAA |
| | | | TGGTGTAATCGAGGCTGACTCCACTTTT | GGAGCAA | GGG |
| | | | AATGTTTGACTGATGATAGGTTTCAAGTC | | |
| | | | TCACTAAGTCTCCTTCCCCTTCTGCCCA | | |
| | | | CATCTCAG | | |
| HEK3_off3 | AGCTCAGACTGAGCAAGTGA | GGG | TGAGAGGGAACAGAAGGGCTAAGACTA | ACACTCTTTCCCTACAC | TGGAGTTCAGACGTG |
| | | | AAAGGAACAGAGGAGTTCATAGTGAGCG | GACGCTCTTCCGATCTN | TGCTCTTCCGATCTGT |
| | | | GTAAAGAGCTCAGACTGAGCAAGTGAG | NNNTGAGAGGGAACAG | CCAAAGGCCCAAGAA |
| | | | GGGCTCAGCCTCCCATGGAGGACAGGG | AAGGGCT | CCT |
| | | | GGCTGGGGCCCCTGGCTGATGTCTGGA | | |
| | | | CTGAAGCCCCCACGCCCAGAGGTTCTT | | |
| | | | GGGCCTTTGGAC | | |
| HEK3_off4 | AGACCAGACTGAGCAAGAGA | GGG | CCTAGCACTTTGGAAGGTCGAAGCGGC | ACACTCTTTCCCTACAC | TGGAGTTCAGACGTG |
| | | | AGGATGGCTTCAACCCAGGAGTTCGAG | GACGCTCTTCCGATCTN | TGCTCTTCCGATCTG |
| | | | ACCAGACTGAGCAAGAGAGGGAGAGTG | NNNTCCTAGCACTTTG | CTCATCTTAATCTGCT |
| | | | TCTGTATTAACAACAAACAAACAAACAAA | GAAGGTCG | CAGCC |
| | | | AAACTAAACTAAAAGAAACTGTGGTGTAT | | |
| | | | AATATAAAATTCTGGCTGAGCAGATTAAG | | |
| | | | ATGAGC | | |
| HEK3_off5 | GAGCCAGAATGAGCACGTGA | GGG | AAAGGAGCAGCTCTTCCTGGTGGAAATT | ACACTCTTTCCCTACAC | TGGAGTTCAGACGTG |
| | | | GCGAGCAGAGGCTGCGTGAGTTCCGTA | GACGCTCTTCCGATCTN | TGCTCTTCCGATCTGT |
| | | | ACTCGCACACAGCCTCCATTTGGAGCCA | NNNAAAGGAGCAGCTC | CTGCACCATCTCCCA |
| | | | GAATGAGCACGTGAGGGACCCCGGGCA | TTCCTGG | CAA |
| | | | GAGGGGCCAGTGCTGACATTATGCTCCA | | |
| | | | TGCAACCTCCCATCCTGTTGTGGGAGAT | | |
| | | | GGTGCAGAC | | |
| HEK4_off1 | TGCACTGCGGCCGGAGGAGG | TGG | GGCATGGCTTCTGAGACTCATAGCTGGG | ACACTCTTTCCCTACAC | TGGAGTTCAGACGTG |
| | | | GCTGAAGATCCCTAGGGGGGCTCTGCT | GACGCTCTTCCGATCTN | TGCTCTTCCGATCTGT |
| | | | GGGCTCACTGCTCTCCAGAGTGGTCCA | NNNGGCATGGCTTCTG | CTCCCTTGCACTCCC |
| | | | GCCCGGCTGCAGGGTGCTGCTTCCAGC | AGACTCA | TGTCTTT |
| | | | TTGGTGCACTGCGGCCGGAGGAGGTGG | | |
| | | | AGGATGGAAAGTAAGATTCAAAGACAGG | | |
| | | | GAGTGCAAGGG | | |
| HEK4_off2 | GGCTCTGCGGCTGGAGGGGG | TGG | TTTGGCAATGGAGGCATTGGGCAGGGG | ACACTCTTTCCCTACAC | TGGAGTTCAGACGTG |
| | | | AAGCCTGTCTTCAGGGCACATGCACGTG | GACGCTCTTCCGATCTN | TGCTCTTCCGATCTGA |
| | | | CGCAGGGCTCTGCGGCTGGAGGGGGT | NNNTTTGGCAATGGAG | AGAGGCTGCCCATGA |
| | | | GGGGTTGCTGTTAGTGACAGGGGCCCC | GCATTGG | GAG |
| | | | AGCCAGGCAGGTTTCAGGATTGGGGAG | | |
| | | | CACTTGCTTCGGCTCCCTTGCTCTCATG | | |
| | | | GGCAGCCTCTTC | | |
| HEK4_off3 | GGCACGACGGCTGGAGGTGG | GGG | GGTCTGAGGCTCGAATCCTGGCAGCAG | ACACTCTTTCCCTACAC | TGGAGTTCAGACGTG |
| | | | GTCCTTCATGGCAAGGCGGGAAAAGAG | GACGCTCTTCCGATCTN | TGCTCTTCCGATCTCT |
| | | | AAAAGCCAACGGGTTCTCATGCTGGGAA | NNNGGTCTGAGGCTCG | GTGGCCTCCATATCC |
| | | | AAGATGCCGGGCACGACGGCTGGAGGT | AATCCTG | CTG |
| | | | GGGGGGTTGGGAGTGGGTGGGATGCTT | | |
| | | | GCGTGCCCTGCATGAGGTGCAGGGATAT | | |
| | | | GGAGGCCACAG | | |
| HEK4_off4 | GGCATCACGGCTGGAGGTGG | AGG | TTCCACCAGAACTCAGCCCAGGCTGCT | ACACTCTTTCCCTACAC | TGGAGTTCAGACGTG |
| | | | GTGGGATGGAATCACCTGCACCCGGAT | GACGCTCTTCCGATCTN | TGCTCTTCCGATCTCC |
| | | | GTTCTTTCTGGGCTGGTACATACAGGCA | NNNTTTCCACCAGAACT | TCGGTTCCTCCACAA |
| | | | AGGCATCACGGCTGGAGGTGGAGGGG | CAGCCC | CAC |
| | | | GCCTAACCCGGGGTTGCCCAGGAAGGG | | |
| | | | GTTTGCACATGGATTCGGTGTGTTGTGG | | |
| | | | AGGAACCGAGG | | |
| HEK4_off5 | GGCGCTGCGGCGGGAGGTGG | AGG | CACGGGAAGGACAGGAGAAGGTGCTGG | ACACTCTTTCCCTACAC | TGGAGTTCAGACGTG |
| | | | ACCGCCTGGACTTTGTGCTGACCAGCCT | GACGCTCTTCCGATCTN | TGCTCTTCCGATCTG |
| | | | TGTGGCGCTGCGGCGGGAGGTGGAGG | NNNCACGGGAAGGACA | CAGGGGAGGGATAAA |
| | | | AGCTGAGAAGCAGCCTGCGAGGGCTTG | GGAGAAG | GCAG |
| | | | CGGGGGAGATTGTTGGGGAGGTCCGGT | | |
| | | | GAGTAATGCGGCTTCTTCTCCTGCTTTAT | | |
| | | | CCCTCCCCTGC | | |
| EMX1_off1 | GAGTTAGAGCAGAAGAAGAA | AGG | TGCCCAATCATTGATGCTTTTATACCATCT | ACACTCTTTCCCTACAC | TGGAGTTCAGACGTG |
| | | | TGGGGTTACAGAAAGAATAGGGGCTTAT | GACGCTCTTCCGATCTN | TGCTCTTCCGATCTAG |
| | | | GGCATGGCAAGACAGATTGTCAGAGTTA | NNNTGCCCAATCATTGA | AAACATTTACCATAGA |
| | | | GAGCAGAAGAAGAAAGGCATGGAGTAAA | TGCTTTT | CTATCACCT |
| | | | GGCAATCTTGTGCAGATGTACAGGTAGC | | |
| | | | AGCCCTCAGAAAAAATAGGTGATAGTCTA | | |
| | | | TGGTAAATGTTTCT | | |
| EMX1_off2 | GAGTCTAAGCAGAAGAAGAA | GAG | GTAGCCTCTTTCTCAATGTGCTTCAACCC | ACACTCTTTCCCTACAC | TGGAGTTCAGACGTG |
| | | | ATCACGGCCTTTGCAAATAGAGCCCTTTA | GACGCTCTTCCGATCTN | TGCTCTTCCGATCTG |
| | | | TTCATAGTAGACAAGAGTCTAAGCAGAA | NNNAGTAGCCTCTTTCT | CTTTCACAAGGATGC |
| | | | GAAGAAGAGAGCCACTACCCAACCATCT | CAATGTGC | AGTCT |
| | | | ACTCTTCTAATGGTGTTTTCCTACAAAGG | | |
| | | | CCAAGTCATGAGACTGCATCCTTGTGAA | | |
| | | | AGC | | |
| EMX1_off3 | GAGGCCGAGCAGAAGAAAGA | OGG | GAGCTAGACTCCGAGGGGAGGCTGCGA | ACACTCTTTCCCTACAC | TGGAGTTCAGACGTG |
| | | | GCCGCAAGCGCAGGAGCCGGGTGGGA | GACGCTCTTCCGATCTN | TGCTCTTCCGATCTTC |
| | | | GAGAGACCCCTTCTTCTGCAAATGAGGA | NNNGAGCTAGACTCCG | CTCGTCCTGCTCTCA |
| | | | GGCCGAGCAGAAGAAAGACGGCGACAG | AGGGGA | CTT |
| | | | ATGTTGGGGGGAGGGGACGGTTTGTGA | | |
| | | | GGGATAGGGAGAGAAAGTCTAAGTGAGA | | |
| | | | GCAGGACGAGGA | | |
| EMX1_off4 | GAGTCCTAGCAGGAGAAGAA | GAG | AGAGGCTGAAGAGGAAGACCAGACTCA | ACACTCTTTCCCTACAC | TGGAGTTCAGACGTG |
| | | | GTAAAGCCTGGAGGCTGCCAGGTAGGG | GACGCTCTTCCGATCTN | TGCTCTTCCGATCTG |
| | | | CTGGGGCCAGCATGACCTGAGTCCTAG | NNNAGAGGCTGAAGAG | GCCCAGCTGTGCATT |
| | | | CAGGAGAAGAAGAGGCAGCCTAGAGTC | GAAGACCA | CTAT |
| | | | TTCTGTGAAGTGCACATAGAAGAGAGAC | | |
| | | | TGGGGCCAAGCCACAAAAGATAGAATGC | | |
| | | | ACAGCTGGGCC | | |
| EMX1_off5 | AAGTCTGAGCACAAGAAGAA | TGG | GTAGTTCTGACATTCCTCCTGAGGGAAA | ACACTCTTTCCCTACAC | TGGAGTTCAGACGTG |
| | | | ATAAATAAATTAATTAAAAATATATATATATA | GACGCTCTTCCGATCTN | TGCTCTTCCGATCTTG |
| | | | TGTATAATGATAAACATGCTAACAAAGTCT | NNNGTAGTTCTGACATT | GTCAATATCTGAAAGG |
| | | | GAGCACAAGAAGAATGGTGAGAAGGAAT | CCTCCTGAG | TTTATTTGT |
| | | | ACATTTTATCTAATAAATATGTAAGCCATTA | | |
| | | | ATAAAATGTAAACCATTAAAACAACAAATA | | |
| | | | AACCTTTCAGATATTGACCA | | |
| EMX1_off6 | GAGTCCGGGAAGGAGAAGAA | AGG | CCAAGAGGGCCAAGTCCTGGCTGTCTG | ACACTCTTTCCCTACAC | TGGAGTTCAGACGTG |
| | | | CCTCTGACGACGAGCAAGGTGGAGGCC | GACGCTCTTCCGATCTN | TGCTCTTCCGATCTCA |
| | | | CTTGGTTAGCAGGATGGGTGGTGAGGA | NNNCCAAGAGGGCCAA | GCGAGGAGTGACAG |
| | | | GTCCGGGAAGGAGAAGAAAGGCTCAGC | GTCCTG | CC |
| | | | GCGGCTTGCCTGAGCCTCCCTCCTCCC | | |
| | | | AGCTCCCGGCCCCTGCTGCCGGCGGCT | | |
| | | | GTCACTCCTCGCTG | | |
| EMX1_off7 | GAGCCGGAGCAGAAGAAGGA | GGG | CACTCCACCTGATCTCGGGGCGCTGTG | ACACTCTTTCCCTACAC | TGGAGTTCAGACGTG |
| | | | CGCTGAGGAAGGCGCGGGCGAGCCGG | GACGCTCTTCCGATCTN | TGCTCTTCCGATCTG |
| | | | AGCAGAAGAAGGAGGGAGGGAGCCAG | NNNCACTCCACCTGAT | GAGGAGGGAGGGAG |
| | | | CCGCTGCAGCCACCACCGCCACCATGT | CTCGGGG | CAG |
| | | | CCTACCAAGGCAAGAAGAACATCCCGC | | |
| | | | GGATCACGGTGAGTCCGGGCGCCGCTG | | |
| | | | CTCCCTCCCTCCTCG | | |
| EMX1_off8 | AAGTCCGAGGAGAGGAAGAA | AGG | ACCACAAATGCCCAAGAGACATCACCAC | ACACTCTTTCCCTACAC | TGGAGTTCAGACGTG |
| | | | TTGGAGAGTCAGAGGTCACAAAAGAGG | GACGCTCTTCCGATCTN | TGCTCTTCCGATCTGA |
| | | | GGCCCAACTCCTGTAGAAGTCCGAGGA | NNNACCACAAATGCCC | CACAGTCAAGGGCCG |
| | | | GAGGAAGAAAGGGTTCTGGAGCTCTCA | AAGAGAC | G |
| | | | GGCGTCAGGGCCAGGCCTGCACCCTTC | | |
| | | | TGTGCCCCTCCATGAATGGCTGGCCGG | | |
| | | | CCCTTGACTGTGTC | | |
| EMX1_off9 | GAATCCAAGCAGGAGAAGAA | GGA | CCCACCTTTGAGGAGGCAAAAGGGAATA | ACACTCTTTCCCTACAC | TGGAGTTCAGACGTG |
| | | | AACTTGTGCTTATTTGTTGGAAGAGCAAA | GACGCTCTTCCGATCTN | TGCTCTTCCGATCTTT |
| | | | TATGTTTTTTTGAAACCGAATTATGGATG | NNNCCCACCTTTGAGG | CCATCTGAGAAGAGA |
| | | | GGGATGTGGGGGTGGGAACTAGGCAAG | AGGCAAA | GTGGT |
| | | | GGTCTCAGGGGAATCCAAGCAGGAGAA | | |
| | | | GAAGGAGGGAAAAACCACTCTCTTCTCA | | |
| | | | GATGGAA | | |
| EMX1_off10 | ACGTCTGAGCAGAAGAAGAA | TGG | GTCATACCTTGGCCCTTCCTCTGTACTCT | ACACTCTTTCCCTACAC | TGGAGTTCAGACGTG |
| | | | ATACAGAGTCCAGCTCTGGCCTGGGAAA | GACGCTCTTCCGATCTN | TGCTCTTCCGATCTTC |
| | | | ATACTTTCAGACAAAACGTCTGAGCAGA | NNNGTCATACCTTGGC | CCTAGGCCCACACCA |
| | | | AGAAGAATGGACAGAACTCTGAGGACAT | CCTTCCT | G |
| | | | TCTTGAGGCACTGGCAGAACCTCTGCA | | |
| | | | GGAAGACGAGAGCATTGCTGGTGTGGG | | |
| | | | CCTAGGGA | | |

TABLE 5
ssDNA oligonucleotide sequences for in vitro and intracellular deamination.

| Name | Sequence | HTS_fwd | HTS_rev |
|---|---|---|---|
| multiC_oligo | AGGTAGTAGAGATGAGTATAGAG | ACACTCT | TGGAGTTCA |
| | GAGTGAAAGCGGAAGTAGGGCC | TTCCCTA | GACGTGTGC |
| | TTCGCGCACCTCATGGAATCCCT | CACGACG | TCTTCCGATC |
| | TCTGCAGCTTCGAACGATCGACC | CTCTTCC | TATCCCTCCT |
| | TGGATCGCTTTTCCGAGCTTCTG | GATCTNN | TCATCTCTAT |
| | GCGGTCTCAAGCACTACCTACGT | NNAGGTA | CTATCTC |
| | CGAGATAGATAGAGATGAAGGAG | GTAGAGA | |
| multiA_oligo | CTGTTGTGTCGTTGTCTGTCGTG | ACACTCTT | TGGAGTTCAG |
| | GTGTGAAAGCGGAAGTAGGGCC | TCCCTACA | ACGTGTGCTC |
| | TTCGCGCACCTCATGGAATCCCT | CGACGCT | TTCCGATCTAG |
| | TCTGCAGCTTCGAACGATCGACC | CTTCCGAT | CACACCAACA |
| | TGGATCGCTTTTCCGAGCTTCTG | CTNNNNCT | GCACGAACG |
| | GCGGTCTCAAGCACTACCTACGT | GTTGTGTC | |
| | CGCGTTCGTTCGTGCTGTTGGT | GTTGTCTG | |
| | | TCGTG | |
| 5′Cy3-singleC_oligo | /5Cy3/ATTATTATTATTTCTATTTATT | N/A | N/A |
| | TATTTATTT | | |

Top to bottom, left to right the sequences correspond to SEQ ID NOs: 204-207.

TABLE 6
On-target editing of whole-genome sequencing samples. The four alleles reflect the
tetrapioid nature of chromosome 1 (which contains the on-target RNF2 locus) in the clonal
population of HEK293T cells that were used for this experiment.

| | Allele 1 | Allele 2 | Allele 3 | Allele 4 |
|---|---|---|---|---|
| BE4-1 | GTTATTTTAGTCATTACCTGAGG | GTTATTTTAGTCATTACCTGAGG | GTTATTTTAGTCATTACCTGAGG | GTCATTTTAGTCATTACCTGAGG |
| BE4-2 | GTTATTTTAGTCATTACCTGAGG | GTTATTTTAGTCATTACCTGAGG | GTTATTTTAGTCATTACCTGAGG | GTCATTTTAGTCATTACCTGAGG |
| BE4-3 | GTTATTTTAGTCATTACCTGAGG | GTTATTTTAGTCATTACCTGAGG | GTTATTTTAGTCATTACCTGAGG | GTTATTTTAGTTATTACCTGAGG |
| BE4-4 | GTTATTTTAGTCATTACCTGAGG | GTTCATTTAGTCATTACCTGAGG | GTCATTTTAGTCATTACCTGAGG | GTCATTTTAGTCATTACCTGAGG |
| BE4-5 | GTTATTTTAGTCATTACCTGAGG | GTTATTTTAGTCATTACCTGAGG | GTTATTTTAGTCATTACCTGAGG | GTCATTTTAGTCATTACCTGAGG |
| BE4-6 | GTTATTTTAGTCATTACCTGAGG | GTTATTTTAGTCATTACCTGAGG | GTCATCTTAGTCATTACCTGAGG | GTCATTTTAGTTATTACCTGAGG |
| BE4-7 | GTTATTTTAGTTATTACCTGAGG | GTTATTTTAGTIATTACCTGAGG | GTCATCTTAGTCATTACCTGAGG | GTCATCTTAATTTTAGTCATTAC |
| | | | | CTGAGG |
| BE4-8 | GTTATTTTAGTCATTACCTGAGG | GTTATTTTAGTCATTACCTGAGG | GTCATTTTAGTCATTACCTGAGG | GTCATTTTAGTTATTACCTGAGG |
| YE1-BE4-1 | GTCATTTTAGTCATTACCTGAGG | GTCATTTTAGTCATTACCTGAGG | GTCATCTTAGTCATTACCTGAGG | GTCATCTTAGTCATTACCTGAGG |
| YE1-BE4-2 | GTCATTTTAGTCATTACCTGAGG | GTCATTTTAGTCATTACCTGAGG | GTCATTTTAGTCATTACCTGAGG | GTCATGTTAGTCATTACCTGAGG |
| YE1-BE4-3 | GTCATTTTAGTCATTACCTGAGG | GTCATTTTAGTCATTACCTGAGG | GTCATTTTAGTCATTACCTGAGG | GTCATTTTAGTCATTACCTGAGG |
| YE1-BE4-4 | GTCATTTTAGTCATTACCTGAGG | GTCATTTTAGTCATTACCTGAGG | GTCATTTTAGTCATTACCTGAGG | GTTATTTTAGTCATTACCTGAGG |
| YE1-BE4-5 | GTCATTTTAGTCATTACCTGAGG | GTCATTTTAGTCATTACCTGAGG | GTCATCTTAGTCATTACCTGAGG | GTCATTTTAGTCATTACCTGAGG |
| YE1-BE4-6 | GTCATTTTAGTCATTACCTGAGG | GTCATTTTAGTCATTACCTGAGG | GTCATCTTAGTCATTACCTGAGG | GTCATTTTAGTCATTACCTGAGG |
| YE1-BE4-7 | GTCATTTTAGTCATTACCTGAGG | GTCATTTTAGTCATTACCTGAGG | GTCATITTAGTCATTACCTGAGG | GTCATTTTAGTCATTACCTGAGG |
| YE1-BE4-8 | GTCATTTTAGTCATTACCTGAGG | GTCATTTTAGTCATTACCTGAGG | GTCATCTTAGTCATTACCTGAGG | GTCATTTTAGTCATTACCTGAGG |
| nCas9-l | GTCATCTTAGTCATTACCTGAGG | GTCATCTTAGTCATTACCTGAGG | GTCATCTTAGTCATTACCTGAGG | GTCATCTTAGTCATTACCTGAGG |
| nCas9-2 | GTCATCTTAGTCATTACCTGAGG | GTCATCTTAGTCATTACCTGAGG | GTCATCTTAGTCATTACCTGAGG | GTCATCTTAGTCATTACCTGAGG |
| nCas9-3 | GTCATCTTAGTCATTACCTGAGG | GTCATCTTAGTCATTACCTGAGG | GTCATCTTAGTCATTACCTGAGG | GTCATCTTAGTCATTACCTGAGG |
| nCas9-4 | GTCATCTTAGTCATTACCTGAGG | GTCATCTTAGTCATTACCTGAGG | GTCATCTTAGTCATTACCTGAGG | GTCATCTTAGTCATTACCTGAGG |
| nCas9-5 | GTCATCTTAGTCATTACCTGAGG | GTCATCTTAGTCATTACCTGAGG | GTCATCTTAGTCATTACCTGAGG | GTCATCTTAGTC------TGAGG |
| nCas9-6 | GTCATCTTAGTCATTACCTGAGG | GTCATCTTAGTCATTACCTGAGG | GTCA------------CCTGAGG | GTCATCT------------GAGG |
| nCas9-7 | GTCATCTTAGTCATTACCTGAGG | GTCATCTTAGTCATTACCTGAGG | GTCATCTTAGTCATTACCTGAGG | GTCATCTTAGTCATTACCTGAGG |
| Parent | GTCATCTTAGTCATTACCTGAGG | GTCATCTTAGTCATTACCTGAGG | GTCATCTTAGTCATTACCTGAGG | GTCATCTTAGTCATTACCTGAGG |

TABLE 7
Average coverage for WGS of each sample.

| Sample Name | Avg. Coverage |
|---|---|
| BE4-1 | 58 |
| BE4-2 | 83 |
| BE4-3 | 93 |
| BE4-4 | 68 |
| BE4-5 | 72 |
| BE4-6 | 85 |
| BE4-7 | 68 |
| BE4-8 | 110 |
| YE1-BE4-1 | 84 |
| YE1-BE4-2 | 78 |
| YE1-BE4-3 | 67 |
| YE1-BE4-4 | 76 |
| YE1-BE4-5 | 78 |
| YE1-BE4-6 | 69 |
| YE1-BE4-7 | 69 |
| YE1-BE4-8 | 72 |
| nCas9-1 | 85 |
| nCas9-2 | 81 |
| nCas9-3 | 62 |
| nCas9-4 | 79 |
| nCas9-5 | 86 |
| nCas9-6 | 72 |
| nCas9-7 | 97 |
| Parent | 55 |

TABLE 8
cDNA amplicon sequences and primers used for amplification for HTS.

| Name | Amplicon | HTS_fwd | HTS_rev |
|---|---|---|---|
| RSL1D1 | TTGGCTTTCCAAATCAGTGGGTCTGACTTGAGGTCTGT | ACACTCTTTCCCT | TGGAGTTCAGACG |
| | GATGTGACCCTTTTCCTCACCTGCTCAACCATTATTCAC | ACACGACGCTCT | TGTGCTCTTCCGA |
| | ATGGACTCCATCATATTCATTTGTAGTCATTCCCAGAGT | TCCGATCTNNNN | TCTCTCATAAGCTT |
| | GGCCCAGTGAGGGTCTCGCTGTATGAGAGTCGGCTAC | TGGCTTTCCAAAT | AGACCAACAAGC |
| | GGAATTTAGGAGAAACAGAAGTTTCTTGGCTTTCATGCT | CAGTGGGTC | |
| | GAGCTTGTTGGTCTAAGCTTATGAG | | |
| CTNNB1 | TTTGATGGAGTTGGACATGGCCATGGAACCAGACAGAA | ACACTCTTTCCCT | TGGAGTTCAGACG |
| | AAGCGGCTGTTAGTCACTGGCAGCAACAGTCTTACCTG | ACACGACGCTCT | TGTGCTCTCCAGC |
| | GACTCTGGAATCCATTCTGGTGCCACTACCACAGCTCC | TCCGATCTNNNN | TACTTGTTCTTGAG |
| | TTCTCTGAGTGGTAAAGGCAATCCTGAGGAAGAGGATG | ATTTGATGGAGTT | TGAAGG |
| | TGGATACCTCCCAAGTCCTGTATGAGTGGGAACAGGGA | GGACATGGCC | |
| | TTTTCTCAGTCCTTCACTCAAGAACAAGTAGCTGG | | |
| IP90 | CTGGTTGACCAATCTGTGGTGAATAGTGGAAATCTGCT | ACACTCTTTCCCT | TGGAGTTCAGACG |
| | CAATGACATGACTCCTCCTGTAAATCCTTCACGTGAAAT | ACACGACGCTCT | TGTGCTCTCTGCG |
| | TGAGGACCCAGAAGACCGGAAGCCCGAGGATTGGGAT | TCCGATCTNNNN | TCTGGATCAGGTA |
| | GAAAGACCAAAAATCCCAGATCCAGAAGCTGTCAAGCC | CTGGTTGACCAA | CG |
| | AGATGACTGGGATGAAGATGCCCCTGCTAAGATTCCAG | TCTGTGGTG | |
| | ATGAAGAGGCCACAAAACCCGAAGGCTGGTTAGATGAT | | |
| | GAGCCTGAGTACGTAC | | |

### Example 3

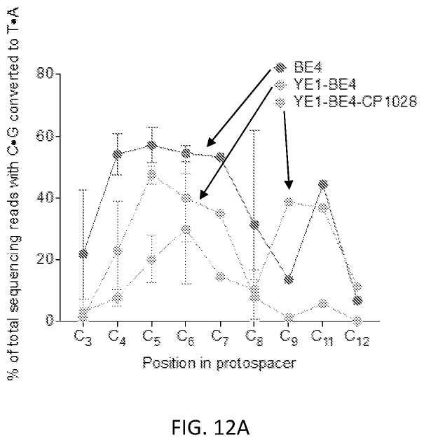

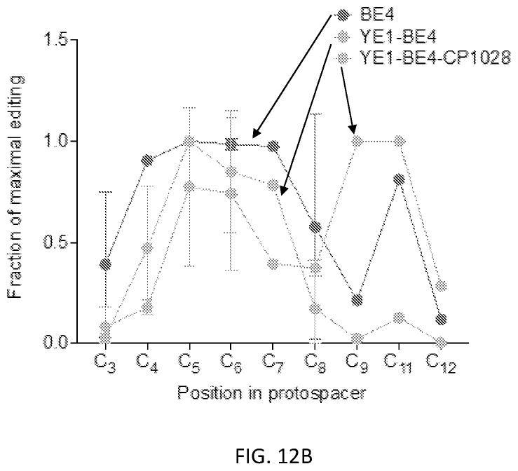

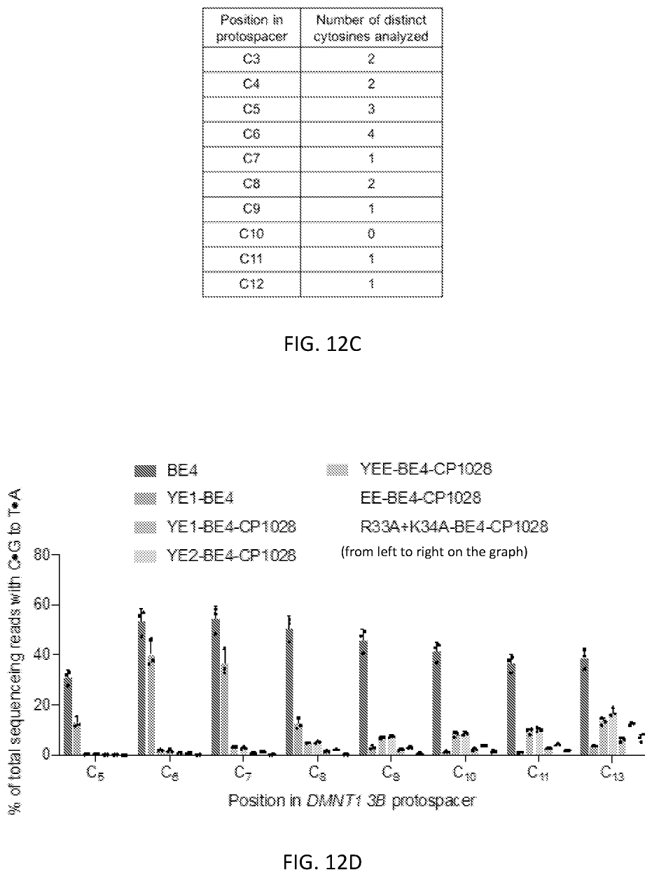

Next, the SpCas9 nickase domain of YE1-BE4, YE2-BE4, YEE-BE4, EE-BE4, and R33A+K34A-BE4 was replaced with CP1028, a circularly permuted SpCas9 variant (43). It has been recently reported that some circularly permutated Cas9 variants can widen or shift the on-target editing window of CBEs and ABEs (5, 43). Indeed, in HEK293T cells at a variety of endogenous loci, it was observed that YE1-BE4-CP1028, YE2-BE4-CP1028, and EE-BE4-CP1028 exhibited base editing activity windows shifted towards the PAM compared to that of non-permuted YE1-BE4 (FIG. 4B and FIG. 11). Collectively, YE1-BE4 and YE1-BE4-CP1028 enable targeting of nearly all cytosines present in the original base editing activity window of BE4, with the exception of sites that contain long multi-C repeats, which for most applications are not considered attractive targets for cytosine base editing regardless of off-target activity (FIGS. 12A-12D). In addition, YEE-BE4-CP1028 and R33A+K34A-BE4-CP1028 were also active at a subset of sites tested and showed shifted editing windows at those sites (FIG. 4B).

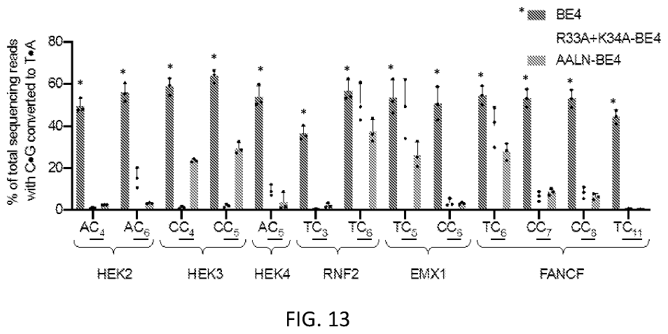

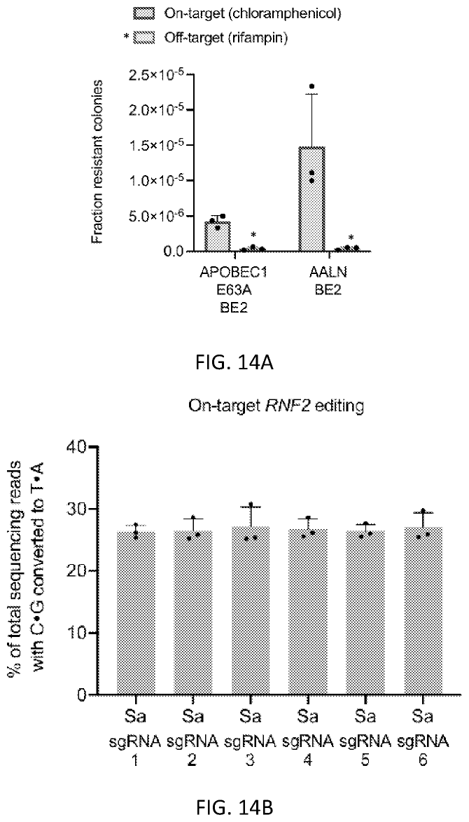

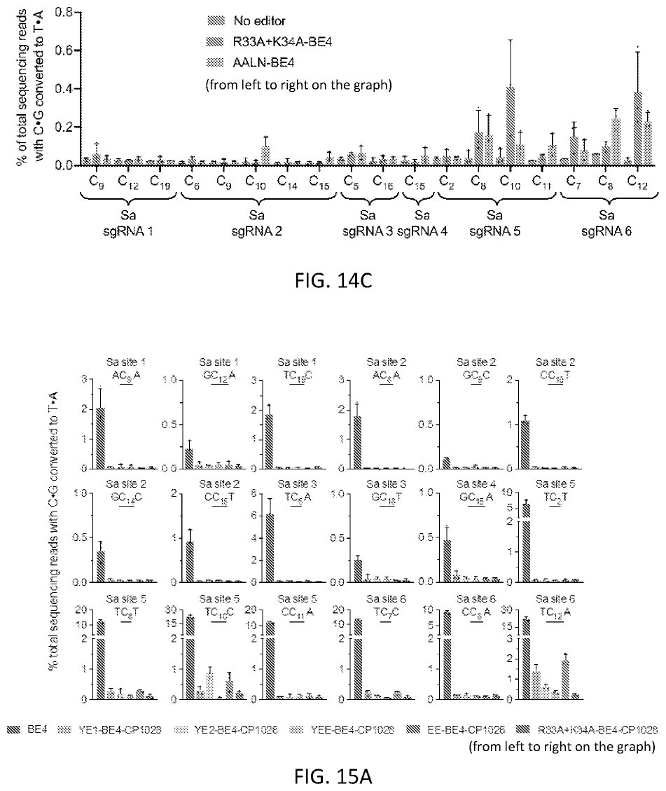

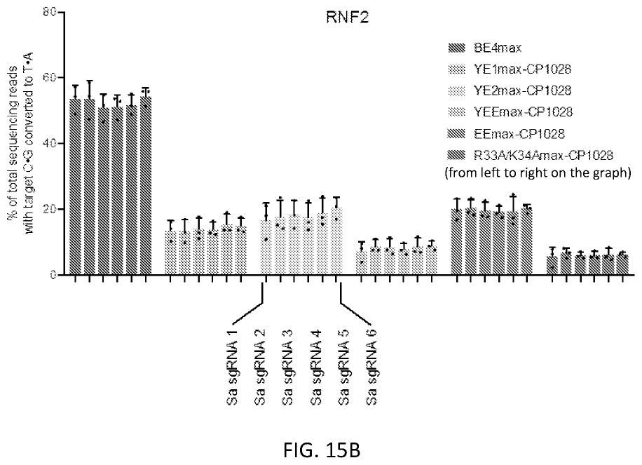

Variants such as YEE-BE4 and R33A+K34A-BE4 are intriguing in that they offer extremely low, if any, off-target deamination in the orthogonal R-loop assay, but they are only active at a subset of on-target sites. To further increase the target sequence compatibility of R33A+K34A-BE4, which exhibits a relatively stringent 5′-TC requirement for base editing, H122L and D124N, two mutations that were found during the continuous evolution of APOBEC1 to enable efficient deamination of 5′-GC substrates (30), were incorporated. The resulting R33A+K34A+H122L+D124N-BE4 variant (referred to as AALN-BE4) indeed changed the profile of targetable C's relative to the original R33A+K34A variant, enabling editing of some positions that were not accessed by R33A+K34A-BE4 (FIG. 13). Importantly, the AALN variant maintains the minimized levels of Cas9-independent deamination shown by R33A+K34A-BE4, and circularly permuted variants likewise displayed Cas9-independent deamination levels equivalent to or lower than their unpermuted counterparts (FIGS. 14A-14C, and FIGS. 15A-15B). This result indicated that deaminases with the lowest number of off-target edits could be engineered to enhance their targeting scope without disrupting their minimal off-target editing profile.

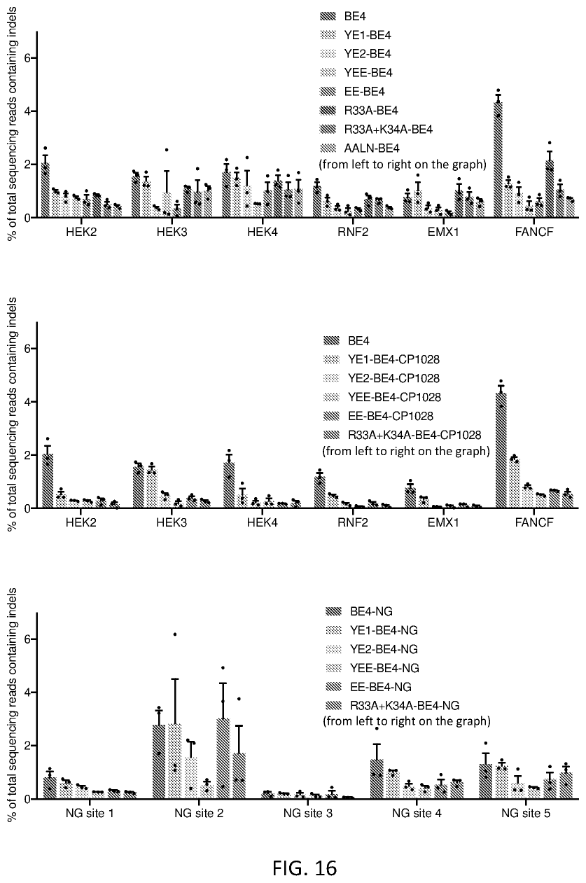

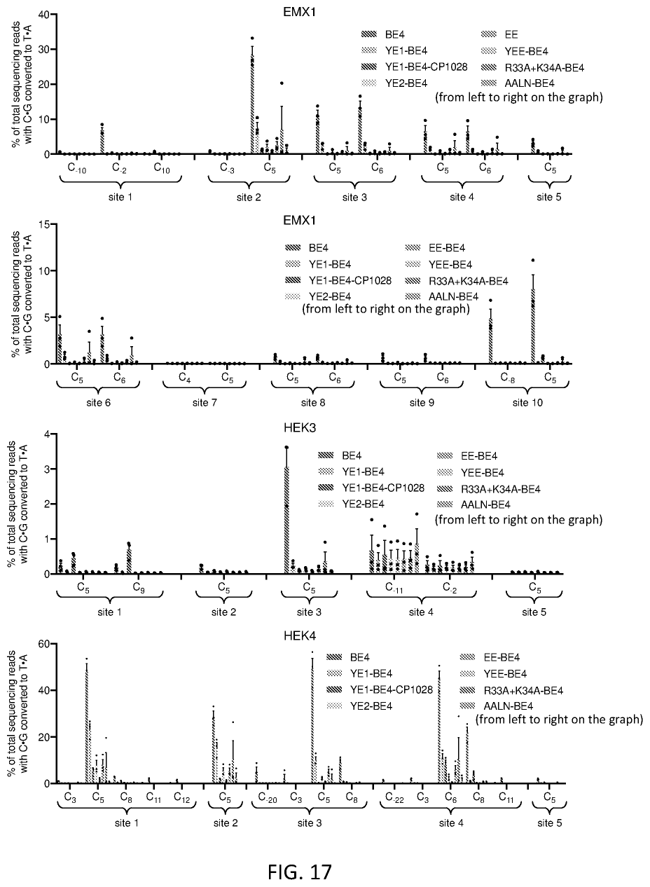

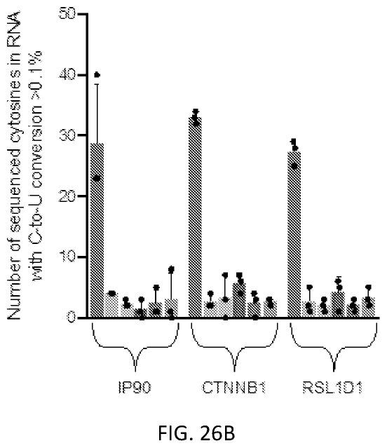

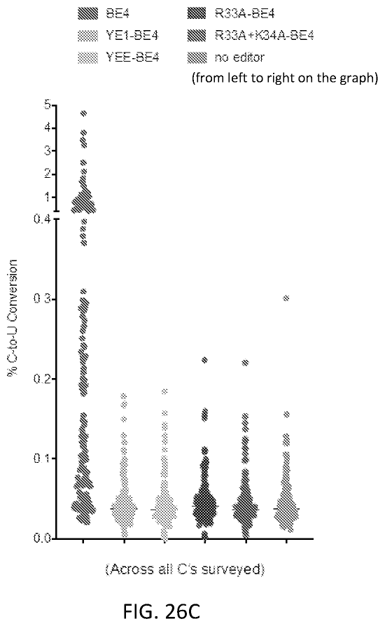

Next, it was assessed if the CBEs that exhibit minimal Cas9-independent deamination have altered propensities to generate other unwanted editing outcomes, such as indels and Cas9-dependent off-target DNA base editing. It was observed that all of these variants (YE1-BE4, YE2-BE4, YEE-BE4, EE-BE4, R33A+K34A-BE4, R33A-BE4, AALN-BE4, the CP1028 variants of the first five of these variants, and the Cas9-NG variants of the same five CBEs), induce lower or comparable levels of indels relative to BE4 across all on-target genomic sites tested (FIG. 16). Moreover, all seven CBE variants (YE1-BE4, YE1-BE4-CP1028, YE2-BE4, EE-BE4, YEE-BE4, R33A+K34A-BE4, and AALN-BE4) that were tested showed much lower levels of Cas9-dependent off-target DNA editing than BE4 when tested at 20 genomic sites previously identified by GUIDE-seq (44) to be the most highly edited off-target substrates of SpCas9 nuclease for three target loci (FIG. 17). In addition, YE1-BE3, R33A-BE3, and R33A+K34A-BE3 were recently found to exhibit substantially reduced levels of transcriptome-wide Cas9-independent RNA off-target editing compared to BE3 (32, 45) (see C. Zhou, et al., Nature 571, 275-278 (2019), herein incorporated by reference). It was confirmed that these variants exhibit decreased Cas9-independent off-target editing of three abundant RNA transcripts, and it was found that YEE also shows decreased RNA off-target editing (FIGS. 26A-26C). These results collectively indicate that the CBEs that minimize Cas9-independent off-target editing do not suffer from higher levels of other forms of unwanted editing; indeed, in general they give rise to fewer indels, less Cas9-dependent DNA off-target editing, and less RNA off-target editing.

Collectively, the expanded targeting capabilities of engineered YE1 variants (YE1-BE4, YE1-CP1028, and YE1-SpCas9-NG) enable targeting, in principle, of 65% of reported pathogenic SNPs that can be corrected by a C:G-to-T:A edit, compared to the only 19% that can be targeted by SpCas9-YE1max alone (FIG. 4C). The known pathogenic SNPs that can be targeted by these engineered CBEs include the vast majority (˜80%) of pathogenic SNPs that can be targeted with the most broadly targetable current-generation BE4max variants, and far outnumber the SNPs targetable by SpCas9-BE4max alone, the most widely-used CBE (FIG. 4C). Therefore, even if a specific target can only be edited to an acceptable level by a BE4-like CBE that uses a deaminase with a high k_{cat}/Kₘ, protein delivery may still provide a path forward to minimize Cas9-independent off target editing.

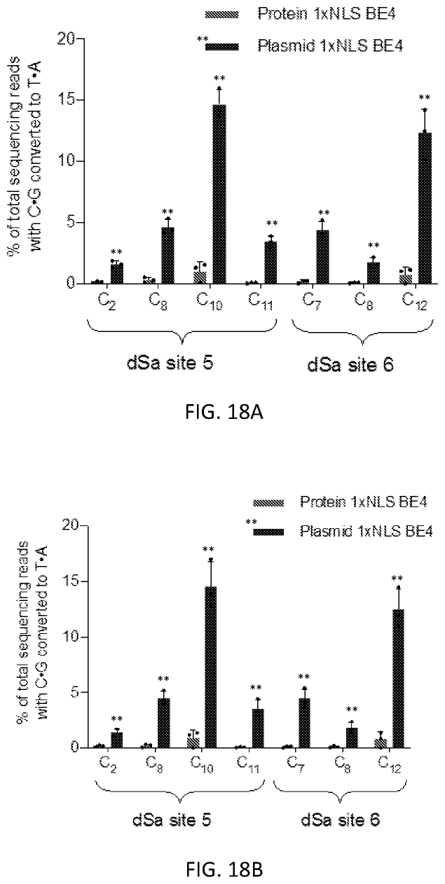

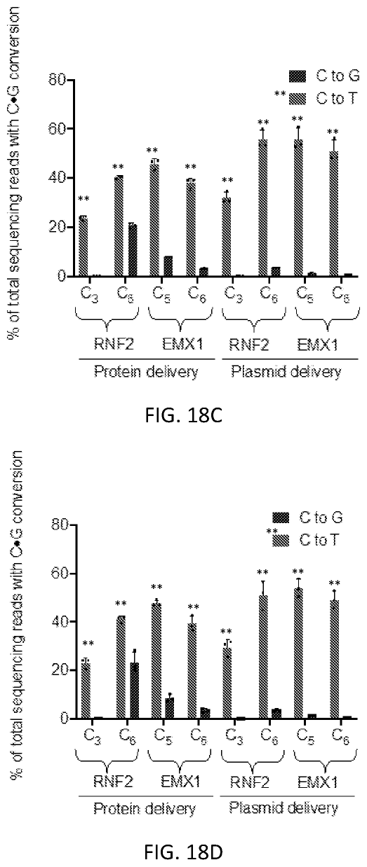

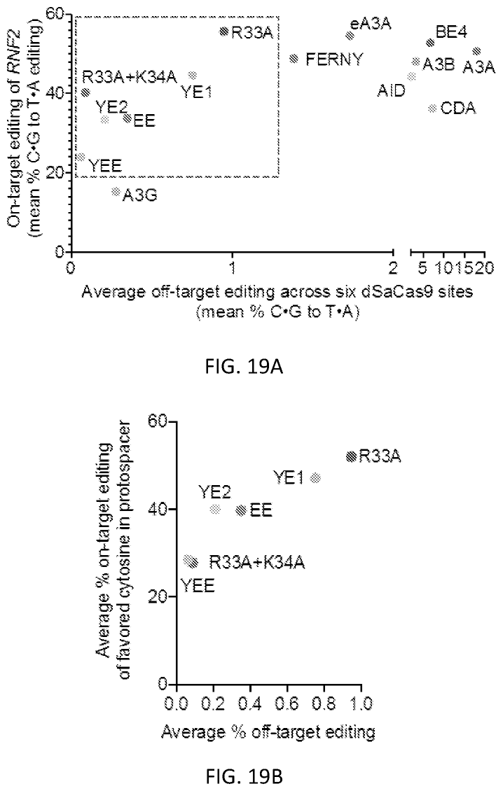

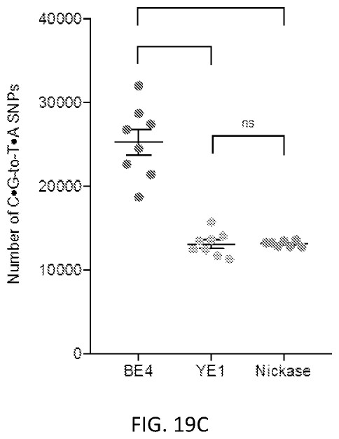

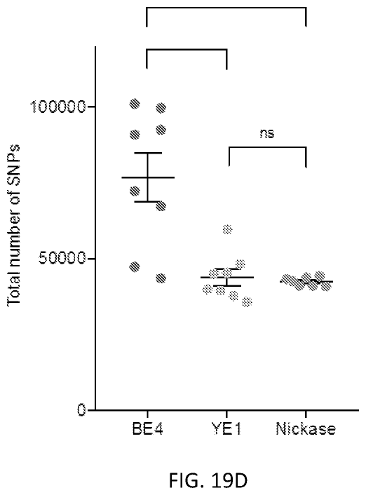

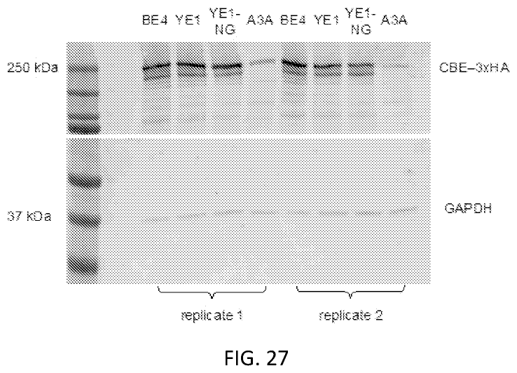

Finally, the manner in which base editor expression and exposure contribute to Cas9-independent off-target editing was explored. Western blots of BE4, YE1-BE4, YE1-SpCas9-NG, and A3A-BE4 revealed that the expression levels of YE1-BE4 and YE1-SpCas9-NG were comparable to that of BE4. However, A3A-BE4 had drastically reduced expression compared to the other three editors, in stark contrast with its higher levels of off-target editing (FIG. 27). A variant of BE4 was then transfected with only one, as opposed to two, nuclear localization signals. This construct should have lower levels of CBE trafficked to the nucleus, and therefore lower effective dosing. Indeed, decreased off-target editing in the R-loop assay was seen when only one NLS was included (FIGS. 18A-18D). This collection of experiments conveys that, while expression of a base editor influences Cas9-independent off-target editing, it cannot fully explain the propensity of an editor to perform Cas9-independent deamination (as shown by A3A-BE4's low expression level but high incidence of Cas9-independent editing). These results also suggested that limiting the time of exposure to the base editor through protein delivery might decrease Cas9-independent off-target editing. Therefore, a 1×NLS-BE4 construct was delivered into HEK293T cells as a protein:RNA complex, and levels of orthogonal R-loop deamination were measured. Average Cas9-independent deamination decreased 21-fold relative to plasmid delivery, while retaining similar on-target editing efficiencies (FIGS. 18A-18D). Therefore, even if a specific target can only be edited to an acceptable level by a BE4-like CBE that uses a deaminase with a high k_{cat}/Kₘ, protein delivery may still provide a path forward to minimize Cas9-independent off target editing.

The assays developed and applied herein enable facile profiling of base editors for Cas9-independent deamination of DNA in bacteria and mammalian cells, and complement in vivo methods such as those performed by Zuo et al. The assays described herein will provide a valuable means of evaluating many CBE variants efficiently and with much lower costs than in vivo experiments that require many whole-genome sequencing experiments (13, 14). The WGS data collected herein validates that these assays are representative of genome-wide off-target DNA mutagenesis rates, and suggests that those CBEs that show low off-target editing in these assays are indeed likely to exhibit low levels of genome-wide off targets. It is anticipated that the assays used here will provide a valuable means of evaluating many CBE variants much more efficiently and with much lower costs than in vivo experiments that require many extensive whole-genome sequencing experiments.

The many deaminases and CBEs characterized and generated herein collectively form a landscape of base editing options with different on-target and off-target editing characteristics, 15 of which are plotted in FIG. 3A. Given this landscape, and the fact that the 5×10⁻⁸/bp mutation rate attributed to Cas9-independent deamination by BE3 in mouse embryos (13) is lower than the observed rate of spontaneous mutation in many mammalian somatic cell types in vivo (46-51), the optimal choice of base editor depends strongly on a given application's on-target sequence context, on-target PAM availability, target tissue type, and the extent to which minimizing low levels of Cas9-independent deamination is critical.

For applications in which off-target editing must be strictly minimized, YE1-BE4, YE2-BE4, YEE-BE4, EE-BE4, R33A+K34A-BE4, YE1-CP1028, YE1-SpCas9-NG, and AALN-BE4 variants are recommended, each of which offer ˜10- to 100-fold lower levels of Cas9-independent off-target DNA editing (FIG. 1 and FIGS. 3A-3D 3), ˜5- to 50-fold lower levels of Cas9-dependent off-target DNA editing (FIG. 17), and lower or similar levels of indel formation (FIG. 16), while maintaining ˜50-90% of average on-target DNA editing levels (FIG. 3A, FIG. 3B, and FIG. 4D) relative to BE4max. Additionally, base editor exposure may also be limited to achieve lower off-target editing. Collectively, the diverse targeting capabilities of this suite of CBEs, especially those that utilize the YE1 deaminase domain, enable high-fidelity base editing at the vast majority of previously accessible target sites with efficiencies approaching those of BE4 (FIG. 4C, FIG. 4D).

### Materials and Methods

Cloning. All plasmids used herein were created using either USER cloning or KLD cloning as described previously (1). DNA was amplified using PhusionU Green Multiplex PCR Master Mix (Thermo Fisher Scientific). Mach1 (Invitrogen) or Turbo (New England BioLabs) chemically competent E. coli were used for plasmid construction.

Preparation and transformation of chemically competent E. coli. Commercially available chemically competent BL21 E. coli (New England BioLabs) were transformed with a plasmid harboring an inactivated chloramphenicol resistance gene. Transformed cells were plated on LB media+1.5% agar supplemented with maintenance antibiotic (kanamycin, 30 μg/mL). The following day, a single colony was picked and grown overnight in 2×YT media supplemented with maintenance antibiotic. The overnight culture was diluted 100-fold into 50 mL of 2×YT media supplemented with maintenance antibiotic and grown at 37° C. with shaking at 230 rpm to OD₆₀₀ ˜0.4-0.6. Cells were collected by centrifugation at 3,400 g for 10 minutes at 4° C. The cell pellet was resuspended by gentle stirring in 2.5 mL of cold LB media followed by 2.5 mL of 2× TSS (LB media supplemented with 5% v/v DMSO, 10% w/v PEG 3350, and 20 mM MgCl₂). After thorough resuspension, cells were aliquoted, frozen on dry ice, and stored at −80° C. until use.

To transform cells, 100 mL of competent cells thawed on ice were added to a pre-chilled mixture of plasmid (2 μL) in 98 μL KCM solution (100 mM KCl, 30 mM CaCl₂), and 50 mM MgCl₂ in H₂O). The mixture was incubated on ice for 20 minutes and heat shocked at 42° C. for 75 seconds followed by addition of 500 μL of SOC media (New England BioLabs). Cells were recovered at 37° C. with shaking at 230 rpm for 1 hour, streaked on 2×YT media+1.5% agar plates containing the appropriate antibiotics, and incubated at 37° C. for 14-16 hours.

Rifampin assay. Chemically competent E. coli harboring a plasmid encoding an inactivated chloramphenicol resistance gene were transformed with a plasmid encoding a base editor+guide RNA. Transformed cells were plated on maintenance antibiotics (30 μg/mL kanamycin, 50 μg/mL spectinomycin, with no chloramphenicol). The following day, colonies were picked and grown overnight in Davis Rich Medium (DRM) and maintenance antibiotics. Overnight cultures were diluted 1:100 into DRM and maintenance antibiotics and grown at 37° C. with shaking at 230 rpm When cells reached OD₆₀₀=0.5, 5 mM rhamnose was added to induce base editor expression. After 18 hours, 700 μL of each culture was centrifuged at 3,400 g for 10 minutes and the cell pellet was resuspended in 150 μL total DRM. Serial dilutions in H₂O of each resuspended culture were plated on three different conditions in parallel: (1) 2×YT agar+30 μg/mL kanamycin+50 μg/mL spectinomycin+20 mM glucose, (2) 2×YT agar+30 μg/mL kanamycin+50 μg/mL spectinomycin+20 mM glucose+100 μg/mL rifampin, or (3) 2×YT agar+30 μg/mL kanamycin+50 μg/mL spectinomycin+20 mM glucose+10 μg/mL chloramphenicol. Surviving colonies were counted following an incubation at 37° C. for 24 hours after plating. To obtain survival rates, the number of colonies in the chloramphenicol or rifampin conditions were divided by the number of colonies counted on the maintenance antibiotic plate.

Sanger sequencing of rpoB mutations from rifampin-resistant colonies. Rifampin-resistant colonies were picked into 10 μL of H₂O and heated at 95° C. for 10 minutes, followed by PCR using primers AB1678 (5′-AATGTCAAATCCGTGGCGTGAC (SEQ ID NO: 208)) and AB1682 (5′-TTCACCCGGATACATCTCGTCTTC (SEQ ID NO: 209)). Each fragment was sequenced twice using primers AB1680 (″-CGGAAGGCACCGTAAAAGACAT (SEQ ID NO: 210)) and AB1683 (5′-CGTGTAGAGCGTGCGGTGAAA (SEQ ID NO: 211)).

HSV thymidine kinase assay. Lambda red recombineering was performed as described previously (52) in order to chromosomally integrate a single copy of the HSV thymidine kinase gene under a constitutive promoter and β-lactamase into the tonB locus of BL21 E. coli. The resulting strain was transformed with a plasmid encoding a base editor and guide RNA. Transformed cells were plated on plasmid maintenance antibiotics (50 μg/mL carbenicillin, 50 μg/mL spectinomycin). The following day, colonies were picked and grown overnight in DRM and maintenance antibiotics. Overnight cultures were diluted 1:100 into DRM and maintenance antibiotics and grown at 37° C. with shaking at 230 rpm. When cells reached OD₆₀₀=0.5, 5 mM rhamnose was added to induce base editor expression. After 18 hours, 700 μL of each culture was centrifuged at 3,400 g for 10 minutes and the cell pellet was resuspended in 150 μL total DRM. Serial dilutions in H₂O of each resuspended culture were plated on two different conditions in parallel: (1) 2×YT agar+50 μg/mL carbenicillin+50 μg/mL spectinomycin+20 mM glucose or (2) 2×YT agar+50 μg/mL carbenicillin+50 μg/mL spectinomycin+20 mM glucose+10 μM 6-(β-D-2-Deoxyribofuranosyl)-3,4-dihydro-8H-pyrimido-[4,5-c][1,2]oxazin-7-one (dP). Surviving colonies were incubated at 37° C. for 24 hours after plating, then counted. To obtain survival rates, the number of colonies in the dP condition was divided by the number of colonies counted on the maintenance antibiotic plate.

Sanger sequencing of HSV thymidine kinase mutations from dP-resistant colonies. dP-resistant colonies were picked into 10 μL of H₂O and heated at 95° C. for 10 minutes, followed by PCR using primers AR393 (5′-AGGCAGTGGGATTGTGGTG) (SEQ ID NO: 230) and AR394 (5′-CGGTCAGCATTAATATTGAAGTGTGG) (SEQ ID NO: 231). Each fragment was sequenced three times using primers AB301 (5′-ATAAAGTTGCAGGACCACTTCT) (SEQ ID NO: 232), AR341 (5′-GCAAGCAGCCCGTAAAC) (SEQ ID NO: 233), and AR392 (5′-CGTACGTCGGTTGCTATG) (SEQ ID NO: 234).

Analysis of BE3-induced point mutations in mouse embryos reported by Zuo et al. (13). Using the genomic locations of all C•G-to-T•A SNVs reported by Zuo et al. (see Tables S6 and S7 of Zuo et al.) the flanking sequences (20 base pairs on either side) were extracted from the mouse mm10 reference genome [GCA_000001635.2]. These flanking sequences were aligned, fixing the mutant cytosine in each case at position 21, and the resulting alignment was used to produce a sequence logo using WebLogo 3.6.0 (53). The custom Python script used for this analysis is included in Supplementary Note 1 below Example 3.

Cell culture. HEK293T cells were maintained in DMEM+GlutaMAX (Life Technologies) supplemented with 10% (v/v) fetal bovine serum. Cells were cultured at 37° C. with 5% carbon dioxide and were confirmed to be negative for mycoplasma by testing with MycoAlert (Lonza Biologics).

Mammalian cell transfections. HEK293T cells were seeded in a 48-well, poly-D-lysine-coated plate (Corning) and transfected at 70% confluence. Plasmids were prepared for transfection using either a ZymoPURE II midi prep kit (Zymo Research Corporation) or a Qiagen midi prep kit (Qiagen). For on-target editing experiments, 750 ng of base editor plasmid and 250 ng of guide RNA plasmid were co-transfected into HEK293T cells using 1.5 μL of Lipofectamine 2000 (ThermoFisher Scientific) per well as directed by the manufacturer. 20 ng of pmaxGFP transfection control plasmid (Lonza Biologics) was used as a transfection control. For orthogonal R-loop assays to measure off-target editing, 200 ng of SpCas9 guide RNA plasmid, 200 ng of SaCas9 guide RNA plasmid, 300 ng of base editor plasmid, and 300 ng of dSaCas9 plasmid were co-transfected into HEK293T cells using 1.5 μL of Lipofectamine 2000. For controls involving no base editor or no sgRNA, pUC19 DNA was used to maintain the total quantity of transfected DNA at 1000 ng. For the intracellular oligonucleotide deamination experiment, 750 ng of base editor plasmid, 250 ng of guide RNA plasmid, and 1 pmol of ssDNA oligonucleotide (Integrated DNA Technologies) were co-transfected into HEK293T cells using 1.5 μL of Lipofectamine 2000.

High-throughput sequencing of genomic DNA. Genomic DNA was sequenced using methods previously described (1). Briefly, genomic DNA was isolated from HEK293T cells three days after transfection. Cells were washed with PBS and then lysed with 150 μL of lysis buffer consisting of 10 mM Tris-HCl (pH 7), 0.05% SDS, and 25 μg/mL of Proteinase K (ThermoFisher Scientific) at 37° C. for 1 hour and then heat inactivated at 80° C. for 30 minutes. Following lysis, 1 L of the genomic DNA lysate was used as input for the first of two PCR reactions. Genomic loci were amplified using a PhusionU PCR kit (Life Technologies) PCR1 primers (“HTS_fwd” and “HTS_rev”) for genomic loci are listed in Table 4. 30 cycles of PCR were performed for all loci with an annealing temperature of 61° C. and an extension time of 30 seconds. For sequencing of the co-transfected ssDNA oligonucleotide, 22 cycles of PCR1 were performed. PCR1 products were confirmed on a 2% agarose gel. 1 μL of PCR1 was used as an input for PCR2 to install Illumina barcodes. PCR2 was conducted using a Phusion HS II kit (Life Technologies). Following PCR2, samples were pooled and gel extracted in a 2% agarose gel using a Qiaquick Gel Extraction Kit (Qiagen). Library concentration was quantified using the Qubit High-Sensitivity Assay Kit (ThermoFisher Scientific). Samples were sequenced on an Illumina MiSeq instrument (paired-end read, R1: 200-280 cycles, R2: 0 cycles) using an Illumina 300 v2 Kit (Illumina).

High-throughput sequencing data analysis. Sequencing reads were demultiplexed using the MiSeq Reporter (Illumina) and fastq files were analyzed using Crispresso2 (see K. Clement et al., CRISPResso2: Accurate and Rapid Analysis of Genome Editing Data from Nucleases and Base Editors. Nature Biotechnology 37, 224-226 (2019), incorporated by reference herein) (54). Representative analysis input and usage are described in Supplementary Note 2 below Example 3. Prism 8 (GraphPad) was used to generate dot plots and bar plots of these data. Base-editing values are representative of n=3 independent biological replicates performed at different times, generally by different researchers, with the mean±SEM shown.

Protein expression and purification for in vitro assays. Base editor purification was performed as described previously (12), with a few modifications. BL21DE3* (ThermoFisher Scientific) chemically competent E. coli were transformed with a plasmid encoding N-terminally 6×His-tagged base editor under control of an IPTG-induced T7 promoter. Individual colonies were picked and grown in 1 L of 2×YT media until OD₆₀₀ ˜0.7-0.8. Cells were cold shocked on ice for 1-2 hours, then induced with 1 mM IPTG (isopropyl-β-D-thiogalactoside; Gold Biotechnology) and grown for a further 12-16 hours at 16° C. with shaking at 220 rpm Cells were collected by centrifugation at 6,000 g for 20 minutes and the resulting cell pellet was resuspended in 25 mL high-salt buffer (100 mM Tris-Cl pH 8.0, 1 M NaCl, 5 mM tris(2-carboxyethyl)phosphine (TCEP; Sigma-Aldrich), 20% glycerol) supplemented with 0.4 mM phenylmethane sulfonyl fluoride (PMSF; Sigma-Aldrich) and EDTA-free protease inhibitor pellet (Roche, 1 pellet per 50 mL lysis buffer used). Cells were lysed by sonication (6 minutes total, 3 seconds on, 3 seconds off) and the lysate was cleared by centrifugation at 22,000 g for 20 minutes. The cleared lysate was incubated with 1.5 mL of TALON Cobalt resin (Clontech) with rotation at 4° C. for 1-2 hours. The resin was washed two times with 15 mL cold high-salt buffer and bound protein was eluted in medium-salt buffer (100 mM Tris-HCl pH 8.0, 0.5 M NaCl, 20% glycerol, 5 mM TCEP) supplemented with 200 mM imidazole. The isolated protein was then buffer-exchanged with low-salt buffer and concentrated using an Amicon Ultra-15 centrifugal filter unit (100,000 molecular weight cutoff). The isolated protein was further purified on a 5 mL Hi-Trap HP SP (GE Healthcare) cation exchange column using an Akta Pure FPLC. Protein-containing fractions were pooled and concentrated using an Amicon Ultra-15 centrifugal filter unit (100,000 molecular weight cutoff). Proteins were quantified using Quick Start Bradford reagent (Bio-Rad) using BSA standards (Bio-Rad) and stored short-term at 4° C.

Protein purity was characterized by SDS-PAGE analysis. Briefly, proteins were denatured at 95° C. for 10 minutes in Laemmli sample loading buffer (Bio-Rad) supplemented with 2 mM dithiothreitol (DTT; Sigma-Aldrich) and separated by electrophoresis at 200 V for 40 minutes on a Bolt 4-12% Bis-Tris Plus (ThermoFisher Scientific) pre-cast gel in Bolt MES SDS running buffer (ThermoFisher Scientific). Gels were stained with InstantBlue reagent (Expedeon) for 1 hour and washed several times with H₂O before imaging with a G: Box Chemi XRQ (Syngene).

In vitro deamination assays. A 5′-Cy3-labeled ssDNA oligonucleotide. (5′-Cy3-ATTATTATTATTTCTATTTATTTATTTATTT (SEQ ID NO: 212)) was purchased as an HPLC-purified oligonucleotide from Integrated DNA Technologies (IDT). All reactions were performed in reaction buffer (20 mM HEPES pH 7.5, 150 mM KCl, 0.5 mM dithiothreitol (DTT), 0.1 mM EDTA, 10 mM MgCl₂) (12) with concentrations of 5′-Cy3-labeled oligonucleotide varying from 0.2-100 μM and concentrations of each purified base editor protein that were >20-fold lower than the substrate concentration assayed in each case. Base editor proteins were incubated at room temperature for 5 minutes with a non-targeting sgRNA added in a 1:1 molar ratio. Subsequently, the 5′-Cy3-labeled oligonucleotide was added to the appropriate concentration and the reactions were incubated at 37° C. for 30 minutes. Reactions were stopped by the addition of buffer PB (100 μL, Qiagen) and isopropanol (25 μL) and purified on a MinElute spin column (Qiagen), eluting in 15 μL of CutSmart buffer (New England BioLabs). USER enzyme (1.5 U, New England BioLabs) was added to the purified ssDNA and incubated at 37° C. for 1 hour. 10 μL of the resulting solution was combined with 10 μL of loading buffer (0.09 M tris(hydroxymethyl)aminomethane, 0.09 M sodium tetraborate, 10 mM EDTA pH 8.0, 10 M urea, 20% sucrose, 0.1% SDS) and loaded on a 10% TBE-urea gel (Bio-Rad) that was pre-run in 0.5× TBE buffer for 15 minutes at 180 V. The cleaved uracil-containing products were resolved from the uncleaved cytosine-containing starting material by electrophoresis for 30 minutes at 180 V, and the gel was imaged on a GE Typhoon FLA 7000 imager. The ratio of product to substrate bands was quantified by densitometry using ImageJ and used to calculate initial reaction velocities. Nonlinear regression was performed using Prism 8 (GraphPad) to fit these data to the Michaelis-Menten equation in order to determine k_{cat} and Kₘ values. Calculation of the propagated error in the k_{cat}/Kₘ ratio from the individual errors in the k_{cat} and Kₘ parameters estimated by the regression is described in Supplementary Note 3 below Example 3.

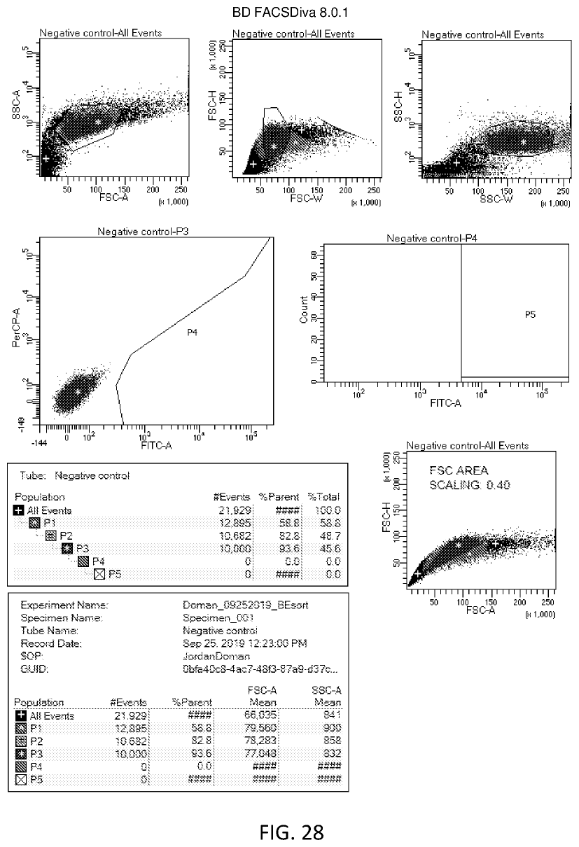

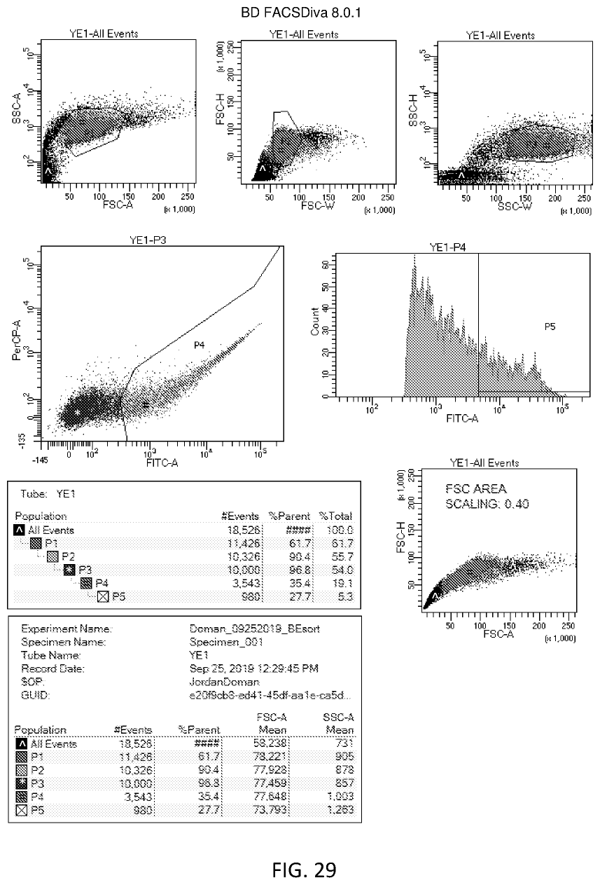

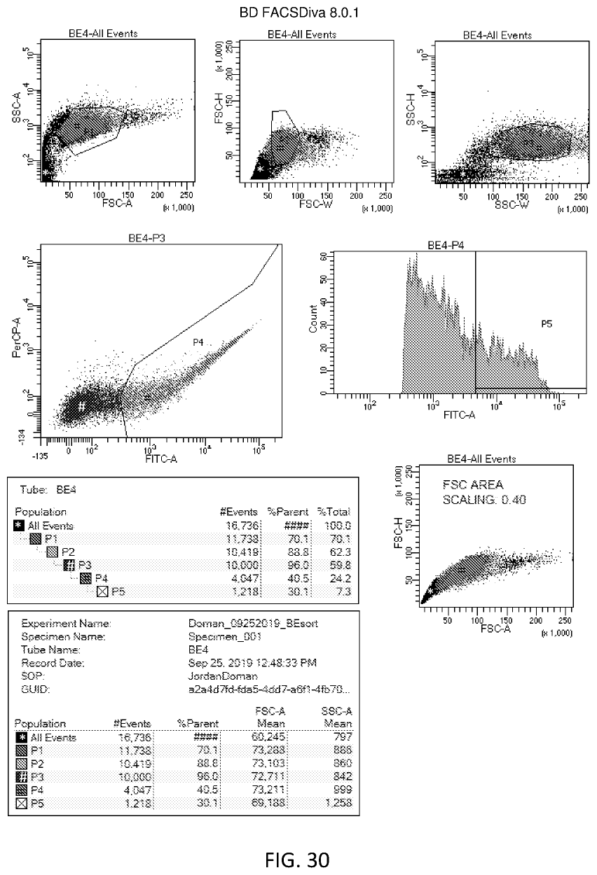

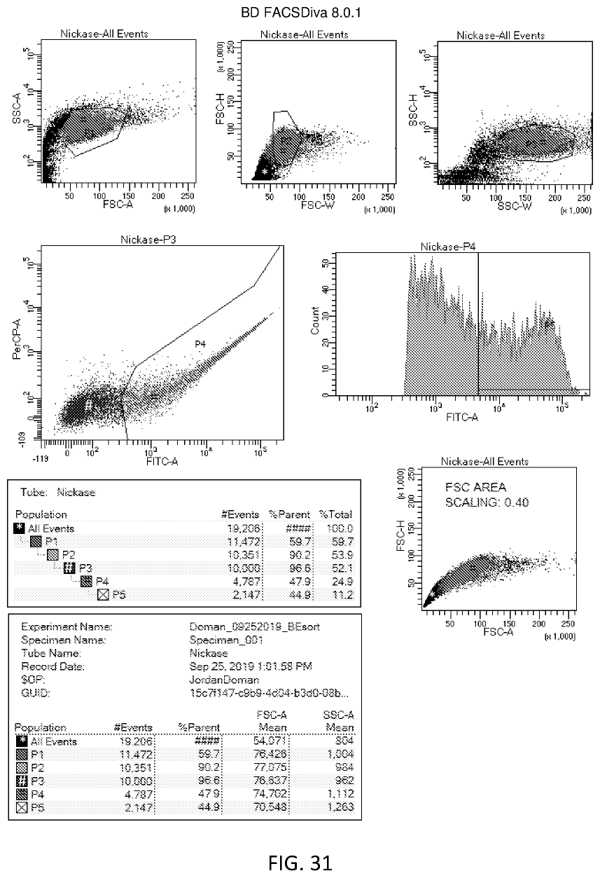

Transfection and fluorescence-activated cell sorting for WGS samples. HEK293T cells were transfected with 750 ng of CBE-P2A-GFP constructs and 250 ng of RNF2-targeting guide RNA as described above. Four wells were transfected for each tested CBE or control (Cas9 nickase instead of a CBE). Four days after transfection, cells were trypsinized with 50 μL of trypsin per well, and resuspended in 200 uL of DMEM (50% FBS (v/v), 100U/mL penicillin/streptomycin). Wells of the same editor were pooled, and cells were filtered through a cell strainer cap (VWR International). Flow sorting was performed on a FACS Aria II (BD Biosciences) sorter using BDFACS Diva software. Cells were gated on forward/side scatter and then gated for GFP signal compared to an untransfected negative control. Cells were then gated on fluorescence intensity. Intensity gates were set to contain the top 28% of GFP-positive YE1-P2A-GFP cell, which corresponded to the top 30% of GFP-positive BE4-P2A-GFP cells and the top 45% of GFP-positive Cas9 nickase-P2A-GFP positives cells (see FIGS. 28-31). Approximately 70,000 cells were collected for each sample in bulk. Of these, about 20,000 cells were sequenced for bulk on-target editing efficiency at the RNF2 locus. The remaining cells were diluted to a concentration of 6 cells/mL (equivalent to 0.9 cells/well) in DMEM (10% FBS (v/v), 100U/mL penicillin-streptomycin). 150 uL of this diluted mixture was pipetted into each well of a 96-well plate. Wells were monitored daily to ensure that each population of cells came from only a single cell. Cells were split into a 48-well, poly-D-lysine-coated plate (Corning) and grown for 16 days before harvesting.

Whole-genome sequencing sample preparation. Cells were lysed using a DNA Agencourt Advance (Beckman Coulter) according to manufacturer instruction. Briefly, 100 μL of lysis buffer (95 μL of Beckman lysis buffer, 2.5 μL of proteinase K (Thermo Fisher), and 2.5 μL of 1M DTT) were added to each well and incubated for 5 min at 37° C. Lysate was then transferred to PCR strips and incubated at 55° C. for 1 hour. 50 μL of Beckman Binding Buffer 1 (Beckman Coulter) was added, and samples were incubated for 2 minutes before the addition of magnetic beads contained in Beckman Binding Buffer 2 (Beckman Coulter). Samples were incubated for 5 minutes and then placed on a magnetic plate for 10 minutes. Supernatant was removed, and beads were washed twice with 70% ethanol. DNA was then resuspended in 50 μL of elution buffer. Samples were placed on a magnetic plate, and the supernatant containing the purified DNA was removed and transferred to fresh tubes. DNA yields were quantified with a Nanodrop. Libraries were created using a Kapa HyperPrep Plus kit according to manufacturer instruction. 800 ng of purified DNA per sample was diluted to a total volume of 35 μL in 10 mM Tris-HCl (pH 8). 5 μL of KAPA frag buffer and 10 μL of Kapa frag enzyme were added to each reaction. Samples were placed in a pre-cooled PCR block and then heated to 37° C. for 12 minutes. Immediately after 12 minutes, samples were placed on ice, and 7 μL of End Repair and A-tailing buffer and 3 μL of End Repair and A-tailing enzyme mix were added immediately to each sample. Samples were mixed and then heated at 20° C. for 30 minutes and then 65° C. for 30 minutes in a thermocycler with the lid temperature set to 85° C. Following this incubation, 10 μL of DNA ligase, 30 μL of DNA ligation buffer, and 10 μL of 15 μM KAPA Adapter primers were added. This mixture was then incubated at 20° C. for 15 minutes. A post-ligation cleanup was performed by adding 88 μL of Kapa Pure beads to the adapter mix. After a 10-minute incubation, beads were collected on a magnetic plate, and the supernatant was discarded. Samples were washed twice with 200 μL of 80% ethanol. Beads were dried for 4 minutes, and 55 μL of elution buffer was added. After incubation, 50 μL of purified DNA was removed from beads.

WGS library size selection and quality control. A size selection was performed on the purified library. A 0.5× cut was performed to remove fragments greater than 1 kb: 25 μL of Kapa Pure beads were added to the eluted library, incubated, and placed on a magnet. The supernatant was collected and saved. 10 μL of fresh Kapa Pure beads were then added to the supernatant to perform a 0.7× second cut. After incubation, libraries were placed on a magnet and the supernatant was removed and discarded. Beads were washed twice with 200 μL of 80% ethanol and then dried for 4 minutes. 40 μL of 10 mM Tris-HCl (pH 8) was added to the beads to elute the final library. Each individual genome was quantified using the Kapa Quantification kit as described previously (1). Library length was determined using an Agilent High Sensitivity DNA Kit and an Agilent 2100 Electrophoresis Bioanalyzer according to manufacturer instructions. Mean fragment length for final libraries was approximately 700 bp.

Whole-genome sequencing and data analysis. Sequencing was performed at the Broad Institute Genomics Platform on an Illumina NovaSeq 6000 using two S4 flow cells. Initial data processing and read alignment was performed by the Broad Institute Genomics Platform. Reads were demultiplexed and aligned to the hg19 (b37) reference genome using BWA-MEM (v0.7.7) (55). Aligned bams were sorted and optical duplicates were marked using Picard tools (v1.1428). Base quality recalibration was performed using GATK (v3.4). All subsequent analyses were performed using the FAS RC Cannon high-performance computing cluster (Harvard University). Sequencing coverage was calculated using mosdepth (v0.2.6) (56). Variant calling on every sample was conducted independently using three algorithms, GATK HaplotypeCaller (v4.1.3.0) (57), freebayes (v1.3.1) (58), and VarScan (v2.4.3) (59), assuming a ploidy of four and a minimum alternate allele read frequency of 0.1 to call an SNV. Bcftools (v1.9) were used to find the intersection of the variants called by all three algorithms in order to generate high-confidence variant calls. For all treated samples, bcftools were used to filter out variants in the treated sample that were present in the parent in order to retain only de novo variants that arose post treatment with base editors. Bcftools were also used to filter out variants present at allele frequencies greater than 0.5 as previously reported (40) in order to restrict analysis to variants that likely arose as a result of base editor treatment. Finally, bcftools were used to exclude variants that exhibited at least one of the following poor quality metrics based on the GATK vcf annotations: QD<2, FS>60, SOR>3, MQ<40, MQRankSum<−5. These final, high-quality variant calls for each treated sample were used for all downstream analyses.

RNA off-target editing analysis. HEK293T cells were transfected with 750 ng of plasmid encoding editors and 250 ng of guide RNA plasmid as described above. Cells were lysed 48 hours after transfection using the RNeasy kit (Qiagen) following manufacturer instructions. Briefly, media was aspirated, and cells were washed with ice cold PBS. To lyse, 350 μL of RLT buffer was added to each well. Cells were pipetted vigorously and then transferred to a DNA eliminator column. Columns were spun at 8000×g for 30 seconds, and 350 μL of 70% ethanol was added to the flow through, which was then applied to an RNeasy spin column. The mixture was centrifuged for 8000×g for 30 seconds. The column was then washed with 700 μL of RW1 buffer and then twice with 500 μL of RPE buffer. The membrane was dried by centrifuging at 8000×g for 1 minute. Purified RNA was eluted with 40 μL of RNase-free water, and 2 μL of RNase-OUT (Fisher Scientific) was added. cDNA was generated using SuperScript IV (Thermo Fisher Scientific). 2 μL of purified RNA was combined with 1 μL of dNTPs, 1 μL of a poly T primer, and 9 μL of RNase-free water. The mixture was heated to 65° C. for 5 minutes and then placed on ice for 1 minute. 4 μL of 5× superscript buffer, 1 μL of SSIV reverse transcriptase 1 μL of 0.1M DTT, and 1 μL of RNase OUT were then added. Two additional reactions were also performed, and reverse transcriptase was not added, as a control for gDNA contamination. Reverse transcription reactions were heated to 50° C. for 10 minutes, then to 80° C. for 10 minutes and then placed on ice. 1 μL of RNAse H was added, and the samples were heated to 37° C. for 20 minutes to degrade RNA. 1 uL of this reaction was used as a template for the first PCR of amplicon sequencing: the remaining protocol is identical to that used for gDNA sequencing. Primers used for each cDNA amplicon and amplicon sequences are listed in Table 8. The no-RT controls were also subjected to Miseq prep, and it was ensured that there were negligible read counts for these samples.

Western blot analysis. HEK293T cells were transfected with 750 ng of plasmid encoding C-terminal 3×HA-tagged base editors and 250 ng of guide RNA plasmid as described above. Cells were lysed 48 hours post transfection at 4° C. for 30 minutes in RIPA buffer (Thermo Fisher) supplemented with 1 mM phenylmethane sulfonyl fluoride (PMSF; Sigma-Aldrich) and EDTA-free protease inhibitor pellet (Roche, 1 pellet per 50 mL lysis buffer used). Lysates were cleared by centrifugation at 12,000 rpm for 20 minutes. Total protein concentration was quantified using Quick Start Bradford reagent (Bio-Rad) using BSA standards (Bio-Rad). Protein extracts were denatured at 95° C. for 10 minutes in Laemmli sample loading buffer (Bio-Rad) supplemented with 2 mM dithiothreitol (DTT; Sigma-Aldrich) and were separated by electrophoresis at 180 V for 40 minutes on a Bolt 4-12% Bis-Tris Plus (ThermoFisher Scientific) pre-cast gel in Bolt MES SDS running buffer (ThermoFisher Scientific). 10 μg of total protein was loaded per well. Transfer to a PVDF membrane was performed using an iBlot 2 Gel Transfer Device (ThermoFisher Scientific) according to the manufacturer's protocols. The membrane was cut in half at the 75 kDa marker and each half was blocked separately in Odyssey Blocking Buffer (LI-COR) in TBS for 1 hour at room temperature with rocking. The high molecular weight half was incubated with rabbit anti-HA (Cell Signaling Technologies 3724S; 1:1000 dilution) in SuperBlock Blocking Buffer (ThermoFisher Scientific) at 4° C. overnight with rocking. The low molecular weight half was incubated with rabbit anti-GAPDH (Cell Signaling Technologies 5174S; 1:1000 dilution) in SuperBlock Blocking Buffer (ThermoFisher Scientific) at 4° C. overnight with rocking. The membranes were washed 2× with TBST (TBS+0.5% Tween-20) for 10 minutes each at room temperature, then incubated with goat anti-rabbit 680RD (LI-COR 926-68071) diluted 1:10,000 in SuperBlock for 1 hour at room temperature. The membrane was washed as before and imaged using an Odyssey Imaging System (LI-COR).

Cell viability assay. HEK293T cells were seeded in a 96-well, clear-bottomed black plate (Corning) and transfected at 70% confluence with 200 ng of base editor plasmid, 40 ng of guide RNA plasmid, and 0.5 μL of Lipofectamine 2000 (ThermoFisher Scientific) per well. 48 or 72 hours post transfection, cell viability was measured using the CellTiter-Glo Reagent (Promega) according to the manufacturer's protocol. Luminescence was measured using an Infinite M1000 Pro microplate reader (Tecan).

Protein nucleofections. To compare on-target editing and off-target editing at orthogonal R-loops using DNA or ribonucleoprotein (RNP) delivery of base editors, cells were first lipofected as described above with the respective plasmids, supplemented to 1000 ng total with pUC19 plasmid if base editor plasmid was not included. 24 hours after lipofection, to allow time for expression of SaCas9 and formation of the R-loop, cells that were treated only with dSaCas9 and orthogonal guide RNA plasmids were trypsinized in 50 μL of TrypLE express (Life Technologies) per well for 5 minutes at 37° C. Cells were suspended and trypsin was quenched with an equal volume of fresh media. Cells were counted in a Countess II cell counter (ThermoFisher Scientific) and 200,000 cells per protein nucleofection sample were apportioned into a single tube. These cells were centrifuged for 8 minutes at 100 g, the supernatant was discarded, and cells were resuspended in 10 L per 200,000 cells of nucleofection solution supplemented as described by the manufacturer (Lonza, SF Cell Line 4D-Nucleofector X Kit S). RNP solutions were prepared by adding 100 pmol of chemically-modified sgRNA (Synthego) to 10 μL of supplemented nucleofection solution per sample. 94 pmol of BE4 protein (expressed and purified by Aldevron, and provided as a generous gift from Prof. Mark Osborn) was added to a final volume of 12 μL, and RNP complexes were formed by incubation at room temperature for 5 minutes. 12 μL of RNP solution was mixed with 200,000 cells in 10 μL of nucleofection solution per sample, and added to a Nucleocuvette (Lonza, SF Cell Line 4D-Nucleofector X Kit S). Cells were nucleofected in a Lonza 4D Nucleofector using program CM-130 according to the manufacturer's instructions. Immediately following nucleofection, cells were recovered for 5 minutes by adding 80 μL of pre-warmed media. 30 μL of recovered cells from each sample were diluted to a final volume of 250 μL in fresh media and incubated at 37° C. for 2 more days before extraction of genomic DNA from all samples, including those treated only with DNA. Three different splits of cells were used in triplicate samples for each treatment.

### Supplementary Note 1:

Custom Python script used for analysis of point mutations (SNVs) reported by Zuo et al. (13). Results of the analysis are depicted as sequence logos in FIGS. 5A-5B.

```
import pandas as pd
import Bio
from Bio import SeqIO
import glob
from Bio.Seq import Seq
from Bio.Alphabet import IUPAC
import glob
import os
from Bio.SeqRecord import SeqRecord
files = [x[5:] for x in glob.glob(‘data/*.csv’)]
chromosomes = [1,2,3,4,5,6,7,8,9,10,11,12,13,14,15,16,17,18,19,‘X’,‘Y’]
os.mkdir(‘data+flanks’)
for file in files:
  data = pd.read_csv(‘data/’ + file)
  flank_seqs = { }
  for chromosome in chromosomes:
    print file, chromosome
    dchr = data.loc[data[‘Chrom’] == ‘chr’ + str(chromosome)]
    record = SeqIO.read(‘mm10/chr’ + str(chromosome) + ‘.fa’, ‘fasta’)
    for index in dchr.index:
      flank_seqs[index] =
str(record.seq[int(dchr.loc[index][‘Pos’])−1−20:int(dchr.loc[index][‘Pos’])−
1+21])
  data[‘Flank_seqs’] = pd.Series(flank_seqs)
  data.to_csv(‘data+flanks/’ + file)
#reverse complement flanks for all G>something SNPs
#remove all nonC>T or G>A SNPs
files = [x[12:] for x in glob.glob(‘data+flanks/*.csv’)]
os.mkdir(‘data+flanks_C20_5p3p’)
for file in files:
  data = pd.read_csv(‘data+flanks/’ + file)
  data.dropna(subset=[‘Mutation’], inplace=True)
  flank_seqs = {int(index) : data.loc[index][‘Flank_seqs’] for index in
data.index}
  #find all G>something SNPs
  gdata = data[data[‘Mutation’].str.contains(‘G>’)]
  #reverse complement flanks for all G>something SNPs
  for index in gdata.index:
    flank_seqs[int(index)] = str(Seq(str(flank_seqs[index]),
IUPAC.unambiguous_dna).reverse_complement( ))
  data[‘Flank_seqs_C20_5p3p’] = pd.Series(flank_seqs)
  data = data[data[‘Mutation’].str.contains(‘C>T|G>A’)]
  data.to_csv(‘data+flanks_C20_5p3p/’ + file)
files = [x[21:] for x in glob.glob(‘data+flanks_C20_5p3p/*.csv’)]
df = pd.DataFrame( )
for file in files:
  df_new = pd.read_csv(‘data+flanks_C20_5p3p/’ + file)
  df = df.append(df_new)
df.drop(columns = [‘Unnamed: 0’, ‘Unnamed: 0.1’], inplace=True)
df.reset_index(inplace=True)
df.drop(columns = [‘index’], inplace=True)
sequences = [ ]
for index in df.index:
  record = SeqRecord(Seq(df.loc[index][‘Flank_seqs_C20_5p3p’],
IUPAC.unambiguous_dna), id=str(index))
  sequences.append(record)
SeqIO.write( sequences,‘data.fasta’,‘fasta’)
```

### Supplementary Note 2:

CRISPResso2 analysis of high-throughput sequencing data. Batch analysis mode (one batch for each unique amplicon and sgRNA combination analyzed) was used in all cases. Reads were filtered by minimum average quality score (phred33) before inclusion in analysis. The output file for each amplicon's results (Reference.NUCLEOTIDE_PERCENTAGE_SUMMARY.txt) were used to quantify C•G to T•A conversion frequencies, either at a subset of C's within the protospacer of interest (on-target editing and off-target editing of dSaCas9 R-loops) or throughout the amplicon (for quantification of deamination of ssDNA in vivo). For indel analysis, a window of 15 bases around the center of the protospacer was used to quantify indels using the output file “CRISPRessoBatch_quantification_of_editing_frequency.txt” for each amplicon.

```
#!/bin/bash
#requires following fastq file format: AMPLICON_sample-xxx.fastq.gz
#requires AR's standard amplicon naming convention
#organize into folders by amplicon
folders=(H2 H3 H4 R2 E1 FF FF1 SaH3)
for folder in ${folders[*]}; do
  mkdir −p $folder
  for file in $( ls ${folder}−* ); do
    mv $file ${folder}/${file}
  done
done
for amplicon in $( ls ); do
  cd $amplicon
  #create batch settings txt file
  echo r1′¥t′n >> ${amplicon}.txt
  #lookup sgRNA and amplicon sequence from standard files
  g=‘cat /guides+amplicons/${amplicon}_sgRNA.txt‘
  a=‘cat /guides+amplicons/${amplicon}_amplicon.txt‘
  #add r1's and names to batch settings txt file
  for sample in $( ls *.gz ); do
    echo ${sample}′¥t′${sample:0:$(expr ${#sample} − 9)} >>
${amplicon}.txt
  done
  #run crispressobatch
  docker run −v ${PWD}:/DATA −w /DATA −i pinellolab/crispresso2
CRISPRessoBatch −−batch_settings ${amplicon}.txt −g $g −a $a −w 30 −wc −10 −q 30
−p 12
  cd ..
done
```

### Supplementary Note 3:

Error propagation for kcat/Km ratio. The error in the kcat/Km ratio determined for each base editor was estimated by propagating the individual SEM values for the kcat and Km parameters estimated by the regression analysis using the following equation (60):

$$Δ ( \frac{{k}_{c a t}}{{K}_{m}} ) = \frac{{k}_{cat}}{{K}_{m}} \sqrt{{( \frac{Δ {k}_{cat}}{{k}_{c a t}} )}^{2} + {( \frac{Δ {K}_{m}}{{K}_{m}} )}^{2}}$$

### Supplementary Note 4:

Calculation of ClinVar pathogenic SNPs targetable by CBEs. The ClinVar database variant summary (updated May 2019) was used in conjunction with the NCBI dbSNP database in order to get flanking genomic contexts for each SNP correctable by a C•G-to-T•A conversion. These genomic contexts were used to define a SNP as targetable by a CBE if a PAM appropriate to that CBE exists that places the SNP in that CBE's editing window.

```
import numpy as np
import pandas as pd
import regex
import re
from Bio import SeqIO
import Bio
import os
###CHANGE PAM AND WINDOW INFO HERE###
PAMs = [‘NGG’]
windows = {‘NGG’: (4,14)}
###CHANGE PAM AND WINDOW INFO HERE###
def RC(seq):
  encoder = {‘A’:‘T’,‘T’:‘A’,‘C’:‘G’,‘G’:‘C’,‘N’:‘N’,‘R’:‘Y’,‘Y’:‘R’,
‘M’:‘K’, ‘K’:‘M’, ‘S’:‘S’, ‘W’:‘W’, ‘H’:‘D’, ‘B’:‘V’, ‘V’:‘B’, ‘D’:‘H’}
  rc = ‘’
  for n in reversed(seq):
    rc += encoder[n]
  return rc
def create_PAM(pam):
  encoder =
{‘A’:‘A’,‘T’:‘T’,‘G’:‘G’,‘C’:‘C’,‘R’:‘[A|G]’,‘Y’:‘[C|T]’,‘N’:‘[A|T|C|G]’,‘M’:‘[A
|C]’,‘K’:‘[G|T]’,‘S’:‘[C|G]’,‘’W’:‘[A|T]’,‘H’:‘[A|C|T]’,‘B’:‘[C|G|T]’,‘V’:‘[A|C|G
]’,‘D’:‘[A|G|T]’}
  enc_pam = {‘f’:‘’,‘r’:‘’}
  rc_pam = RC(pam)
  for n,m in zip(pam, rc_pam):
    enc_pam[‘f’] += encoder[n]
    enc_pam[‘r’] += encoder[m]
  return enc_pam
ClinVar=pd.read_csv(‘2019-05-06-variant_summary.csv’, encoding = “ISO-8859-1”)
Phenotypes=pd.read_csv(‘DiseaseNames.csv’)
PhenotypeDict=dict(zip(Phenotypes.CUI, Phenotypes.name))
#open flanking sequence fasta files for all Y-type pathogenic human SNPs
(includes both C>T and T>C ref>variant)
#downloaded as fasta file from:
#http://www.ncbi.nlm.nih.gov/snp/?term=((%22pathogenic %22%5BClinical+Significanc
e%5D+AND+%22snp%22%5BSNP+Class%5D+AND+homo+sapiens%5BOrganism %5D+)+AND(%22y%22%5
BAllele%5D))
handle = open(“YFasta.txt”, “rU”)
flanks={ }
#save as a dictionary keyed on rsID as an Integer with values being 25nt of
flanking sequence on each side of the SNP
for record in SeqIO.parse(handle, “fasta”) :
  flanks[int(record.id.split(“|”)[−
1].strip(‘rs’))]=regex.findall(‘.{25}[\hat{ }A,T,C,G].{25}’, record.seq.tostring( ) )
handle.close( )
# clinvar may refer to the opposite strand that was used in dbSNP;
# we want to allow clinvar reference alleles A and T with alternate alleles G
and C respectively
# we do not want to allow reference alleles G and C with alternate alleles A and
T respectively; these Y-type SNPs must be removed
ClinVar_mod=ClinVar[(ClinVar.ReferenceAllele==‘A’) |
(ClinVar.ReferenceAllele==‘T’)].drop_duplicates(‘#AlleleID’)
#merge flanking sequences to the CtoT frame on rsID
F=pd.DataFrame({‘RS# (dbSNP)’: list(flanks.keys( )), ‘Flanks’: [x for x in
flanks.values( )]})
CtoT=F.merge(ClinVar_mod, left_on=‘RS# (dbSNP)’, right_on=‘RS# (dbSNP)’,
how=‘inner’)
#open flanking sequence fasta files for all R-type pathogenic human SNPs
(includes both G>A and A>G ref>variant)
#downloaded as fasta file from:
#http://www.ncbi.nlm.nih.gov/snp/?term=((%22pathogenic%22%5BClinical+Significanc
e%5D+AND+%22snp%22%5BSNP+Class%5D+AND+homo+sapiens%5BOrganism%5D+)+AND(%22r%22%5
BAllele%5D))
handle = open(“RFasta.txt”, “rU”)
flanks={ }
#save as a dictionary keyed on rsID as an Integer with values being 25nt of
flanking sequence on each side of the SNP
for record in SeqIO.parse(handle, “fasta”) :
  flanks[int(record.id.split(“|”)[−
1].strip(‘rs’))]=regex.findall(‘.{25}[\hat{ }A,T,C,G].{25}’, record.seq.tostring( ))
handle.close( )
#merge flanking sequences to the CtoT frame on rsID
F=pd.DataFrame({‘RS# (dbSNP)’: list(flanks.keys( )), ‘Flanks’: [x for x in
flanks.values( )]})
GtoA=F.merge(ClinVar_mod, left_on=‘RS# (dbSNP)’, right_on=‘RS# (dbSNP)’,
how=‘inner’)
#empty lists to later combine data for all PAMs
hasPAM_CtoT_dfs =[ ]
hasPAM_GtoA_dfs =[ ]
singleC_CtoT_dfs = [ ]
singleC_GtoA_dfs = [ ]
os.chdir(‘output’)
for PAM in PAMs:
  #define window limits and the length of the pam including all N residues
  enc_pam = create_PAM(PAM)
  windowstart = windows[PAM][0]
  windowend = windows[PAM][1]
  windowlen=windowend−windowstart+1
  lenpam=len(PAM)
  CtoTmod = CtoT
  CtoTmod[‘gRNAs’]=None
  CtoTmod[‘gRNAall’]=None
  for i in range(len(CtoTmod)):
    print i
    if type(CtoTmod.iloc[i].Flanks)==list and
CtoTmod.iloc[i].Flanks!=[ ]:
      test=CtoTmod.iloc[i].Flanks[0]
      # define a potential gRNA spacer for each window
positioning
      gRNAoptions=[test[(26−windowstart−j):(26−windowstart−
j+lenpam+20)] for j in range(windowlen)]
      #if there is an appropriate PAM placed for a given gRNA
spacer
      #save tuple of gRNA spacer, and the position of off-target
Cs in the window
  gRNA=[(gRNAoptions[k],[x.start( )+1 for x in
re.finditer(‘C’,gRNAoptions[k]) if windowstart−1<x.start( )+1<windowend+1]) for k
in range(len(gRNAoptions)) if regex.match(enc_pam[‘f’], gRNAoptions[k][−
lenpam:])]
      gRNAsingleC=[ ]
      for g,c in gRNA:
        #if the target C is the only C in the window save
this as a single C site
        if g[windowstart−1:windowend].count (‘C’)==0:
          gRNAsingleC.append(g)
        #OPTIONAL uncomment the ELIF statement if you are
interest in filtered based upon position of off-target C
        #if the target C is expected to be editted more
efficiently than the off-target Cs, also save as a single C Site
        #elif all([p<priority[x] for x in c]):
          #gRNAsingleC.append(g)
      CtoTmod.gRNAs.iloc[i]=gRNAsingleC
      CtoTmod.gRNAall.iloc[i]=[g for g,c in gRNA]
  GtoAmod = GtoA
  GtoAmod[‘gRNAs’]=None
  GtoAmod[‘gRNAall’]=None
  for i in range(len(GtoAmod)):
    print i
    if type(GtoAmod.iloc[i].Flanks)==list and
GtoAmod.iloc[i].Flanks!=[ ]:
      test=Gt:oAmod.iloc[i].Flanks[0 ]
      gRNAoptions=[test[(25+windowstart+j−20−
lenpam):(25+windowstart+j)] for j in range(windowlen)]
      gRNA=[(gRNAoptions[k],[20+lenpam−x.start( ) for x in
re.finditer(‘G’,gRNAoptions[k]) if windowstart−1<20+lenpam−
x.start( )<windowend+1]) for k in range(len(gRNAoptions)) if
regex.match(enc_pam[‘r’], gRNAoptions[k][:lenpam])]
      gRNAsingleC=[ ]
      for g,c in gRNA:
        if g[20+lenpam−windowstart−windowlen+1:20+lenpam−
windowstart+1].count(‘G’)==0:
          gRNAsingleC.append(g)
        #elif all([p<priority[x] for x in c]):
          #gRNAsingleC.append(g)
      GtoAmod.gRNAs.iloc[i]=gRNAsingleC
      GtoAmod.gRNAall.iloc[i]=[g for g,c in gRNA]
  #merge in phenotypes based upon MedGen IDs; remove redundant columns
  CtoTmod=CtoTmod[[‘RS# (dbSNP)’,‘GeneSymbol’,‘Name’, ‘PhenotypeIDs’,
‘Origin’, ‘ReviewStatus’, ‘NumberSubmitters’, ‘LastEvaluated’, ‘gRNAs’,
‘gRNAall’]]
  ids=[re.findall(‘MedGen:C.{7}’, x) for x in CtoTmod.PhenotypeIDs.values]
  CtoTmod[‘Phenotypes']=[[PhenotypeDict[y.lstrip(‘MedGen:’)] for y in x if
y.lstrip(‘MedGen:’) in PhenotypeDict.keys( )] for x in ids]
  CtoTmod.drop(‘PhenotypeIDs', inplace=True, axis=1)
  GtoAmod=GtoAmod[[‘RS# (dbSNP)’,‘GeneSymbol’, ‘Name’, ‘PhenotypeIDs’,
‘Origin’, ‘ReviewStatus’, ‘NumberSubmitters’, ‘LastEvaluated’, ‘gRNAs’,
‘gRNAall’]]
  ids=[re.findall(‘MedGen:C.{7}’, x) for x in GtoAmod.PhenotypeIDs.values]
  GtoAmod[‘Phenotypes’]=[[PhenotypeDict[y.lstrip(‘MedGen:’)] for y in x if
y.lstrip(‘MedGen:’) in PhenotypeDict.keys( )] for x in ids]
  GtoAmod.drop(‘PhenotypeIDs’, inplace=True, axis=1)
  CtoTmod.to_csv(‘pathogenic_CtoT_all.csv’)
  GtoAmod.to_csv(‘pathogenic_GtoA_all.csv’)
  pathogenic_CtoT_hasPAM=CtoTmod[[type(x)==list and x!=[ ] for x in
CtoTmod.gRNAall]]
  pathogenic_GtoA_hasPAM=GtoAmod[[type(x)==list and x!=[ ] for x in
GtoAmod.gRNAall]]
  pathogenic_GtoA_hasPAM.to_csv(‘pathogenic_GtoA_has_’+PAM+‘_PAM.csv’)
  pathogenic_CtoT_hasPAM.to_csv(‘pathogenic_CtoT_has_’+PAM+‘_PAM.csv’)
  hasPAM_CtoT_dfs.append(pathogenic_CtoT_hasPAM)
  hasPAM_GtoA_dfs.append(pathogenic_GtoA_hasPAM)
  pathogenic_CtoT_SingleC=CtoTmod[[type(x)==list and x!=[ ] for x in
CtoTmod.gRNAs]]
  pathogenic_GtoA_SingleC=GtoAmod[[type(x)==list and x!=[ ] for x in
GtoAmod.gRNAs]]
  pathogenic_GtoA_SingleC.to_csv(‘pathogenic_GtoA_’+PAM+‘_PAM_SingleC.csv’)
  pathogenic_CtoT_SingleC.to_csv(‘pathogenic_CtoT_’+PAM+‘_PAM_SingleC.csv’)
  singleC_CtoT_dfs.append(pathogenic_CtoT_SingleC)
  singleC_GtoA_dfs.append(pathogenic_GtoA_SingleC)
  with open(‘Summary_’+PAM+‘.txt’, “w”) as text_file:
    text_file.write(“singleC %s \n” %
(len(pathogenic_CtoT_SingleC)+len(pathogenic_GtoA_SingleC)))
    text_file.write(“hasPAM %s \n” %
(len(pathogenic_CtoT_hasPAM)+len(pathogenic_GtoA_hasPAM)))
    text_file.write(“Pathogenic SNPs that can be targeted with BE %s”
% (len(CtoTmod)+len(GtoAmod)))
hasPAM_CtoT_alIPAMs = pd.concat(hasPAM_CtoT_dfs)
hasPAM_GtoA_allPAMs = pd.concat(hasPAM_GtoA_dfs)
singleC_CtoT_allPAMs = pd.concat(singleC_CtoT_dfs)
singleC_GtoA_allPAMs = pd.concat(singleC_GtoA_dfs)
#remove duplicates
hasPAM_CtoT_allPAMs =
hasPAM_CtoT_allPAMs[~hasPAM_CtoT_allPAMs.index.duplicated(keep=‘first’)]
hasPAM_GtoA_allPAMs =
hasPAM_GtoA_allPAMs[~hasPAM_GtoA_allPAMs.index.duplicated(keep=‘first’)]
singleC_CtoT_allPAMs =
singleC_CtoT_allPAMs[~singleC_CtoT_allPAMs.index.duplicated(keep=‘first’)]
singleC_GtoA_allPAMs =
singleC_GtoA_allPAMs[~singleC_GtoA_allPAMs.index.duplicated(keep=‘first’)]
with open(‘Summary_allPAMs.txt’, “w”) as text_file:
  text_file.write(“singleC %s \n” %
(len(singleC_CtoT_allPAMs)+len(singleC_GtoA_allPAMs)))
  text_file.write(“hasPAM %s \n” %
(len(hasPAM_CtoT_allPAMs)+len(hasPAM_GtoA_allPAMs)))
  text_file.write(“Pathogenic SNPs that can be targeted with BE %s” %
(len(CtoTmod)+len(GtoAmod)))
```

### REFERENCES

1. A. C. Komor et al., Programmable editing of a target base in genomic DNA without double-stranded DNA cleavage. Nature 533, 420-424 (2016).
2. A. C. Komor et al., Improved base excision repair inhibition and bacteriophage Mu Gam protein yields C:G-to-T:A base editors with higher efficiency and product purity. Science Advances 3, eaao4774 (2017).
3. A. C. Komor, A. H. Badran, D. R. Liu, CRISPR-Based Technologies for the Manipulation of Eukaryotic Genomes. Cell 168, 20-36 (2017).
4. H. A. Rees, D. R. Liu, Base editing: precision chemistry on the genome and transcriptome of living cells. Nat Rev Genet 19, 770-788 (2018).
5. M. Paz Zafra et al., Optimized base editors enable efficient editing in cells, organoids and mice. Nature Biotechnology 36, 888-893 (2018).
6. K. Kim et al., Highly efficient RNA-guided base editing in mouse embryos. Nat Biotechnol 35, 435-437 (2017).
7. Y. Zhang et al., Programmable base editing of zebrafish genome using a modified CRISPR-Cas9 system. Nat Commun 8, 118 (2017).
8. Y. Zong et al., Precise base editing in rice, wheat and maize with a Cas9-cytidine deaminase fusion. Nat Biotechnol 35, 438-440 (2017).
9. N. M. Gaudelli et al., Programmable base editing of A*T to G*C in genomic DNA without DNA cleavage. Nature 551, 464-471 (2017).
10. D. Kim et al., Genome-wide target specificities of CRISPR RNA-guided programmable deaminases. Nat Biotechnol 35, 475-480 (2017).
11. P. Liang et al., Genome-wide profiling of adenine base editor specificity by EndoV-seq. Nature Communications 10, (2019).
12. H. A. Rees et al., Improving the DNA specificity and applicability of base editing through protein engineering and protein delivery. Nat Commun 8, 15790 (2017).
13. E. S. Zuo et al., Cytosine base editor generates substantial off-target single-nucleotide variants in mouse embryos. Science 364, 289-292 (2019).
14. S. Z. Jin et al., Cytosine, but not adenine, base editors induce genome-wide off-target mutations in rice. Science 364, (2019).
15. A. H. Badran, D. R. Liu, Development of potent in vivo mutagenesis plasmids with broad mutational spectra. Nat Commun 6, 8425 (2015).
16. L. Garibyan, Use of the rpoB gene to determine the specificity of base substitution mutations on the Escherichia coli chromosome. DNA Repair 2, 593-608 (2003).
17. R. S. Harris et al., RNA Editing Enzyme APOBEC1 and Some of Its Homologs Can Act as DNA Mutators. Molecular Cell 10, 1247-1253 (2002).
18. R. M. Kohli et al., A portable hot spot recognition loop transfers sequence preferences from APOBEC family members to activation-induced cytidine deaminase. J Biol Chem 284, 22898-22904 (2009).
19. H. Lee et al., Foster, Rate and molecular spectrum of spontaneous mutations in the bacterium Escherichia coli as determined by whole-genome sequencing. Proc Natl Acad Sci USA 109, E2774-2783 (2012).
20. K. Fukui, DNA mismatch repair in eukaryotes and bacteria. J Nucleic Acids 2010, (2010).
21. G. Saraconi et al., The RNA editing enzyme APOBEC1 induces somatic mutations and a compatible mutational signature is present in esophageal adenocarcinomas. Genome Biology 15, (2014).
22. K. Nishida et al., Targeted nucleotide editing using hybrid prokaryotic and vertebrate adaptive immune systems. Science 353, aaf8729-aaf8729 (2016).
23. Y. Ma et al., Targeted AID-mediated mutagenesis (TAM) enables efficient genomic diversification in mammalian cells. Nat Methods 13, 1029-1035 (2016).
24. G. T. Hess et al., Directed evolution using dCas9-targeted somatic hypermutation in mammalian cells. Nat Methods 13, 1036-1042 (2016).
25. X. Wang et al., Efficient base editing in methylated regions with a human APOBEC3A-Cas9 fusion. Nat Biotechnol 36, 946-949 (2018).
26. M. A. Coelho et al., BE-FLARE: a fluorescent reporter of base editing activity reveals editing characteristics of APOBEC3A and APOBEC3B. BMC Biol 16, 150 (2018).
27. A. St Martin et al., A fluorescent reporter for quantification and enrichment of DNA editing by APOBEC-Cas9 or cleavage by Cas9 in living cells. Nucleic Acids Res 46, e84 (2018).
28. A. St Martin et al., A panel of eGFP reporters for single base editing by APOBEC-Cas9 editosome complexes. Sci Rep 9, 497 (2019).
29. Z. Liu et al., Highly precise base editing with CC context-specificity using engineered human APOBEC3G-nCas9 fusions. Bioarchive, (2019).
30. B. W. Thuronyi, et al., Continuous evolution of base editors with expanded target compatibility and improved activity. Nature Biotechnology 37, 1070-1079 (2019).
31. Y. B. Kim et al., Increasing the genome-targeting scope and precision of base editing with engineered Cas9-cytidine deaminase fusions. Nat Biotechnol 35, 371-376 (2017).
32. J. Grunewald et al., Transcriptome-wide off-target RNA editing induced by CRISPR-guided DNA base editors. Nature 569, 433-437 (2019).
33. J. M. Gehrke et al., An APOBEC3A-Cas9 base editor with minimized bystander and off-target activities. Nature Biotechnology 36, 977-982 (2018).
34. Y. Tashiro et al., A nucleoside kinase as a dual selector for genetic switches and circuits.

Nucleic Acids Res 39, e12 (2011).
35. L. W. Koblan et al., Improving cytidine and adenine base editors by expression optimization and ancestral reconstruction. Nature Biotechnology 36, 843-846 (2018).
36. K. Chan et al., An APOBEC3A hypermutation signature is distinguishable from the signature of background mutagenesis by APOBEC3B in human cancers. Nat Genet 47, 1067-1072 (2015).
37. T. Eto et al., RNA-editing cytidine deaminase Apobec-1 is unable to induce somatic hypermutation in mammalian cells. PNAS 100, 12895-12898 (2003).
38. M. A. Carpenter et al., Methylcytosine and normal cytosine deamination by the foreign DNA restriction enzyme APOBEC3A. J Biol Chem 287, 34801-34808 (2012).
39. L. Lei et al., APOBEC3 induces mutations during repair of CRISPR-Cas9-generated DNA breaks. Nat Struct Mol Biol 25, 45-52 (2018).
40. M. K. Akre et al., Mutation Processes in 293-Based Clones Overexpressing the DNA Cytidine deaminase APOBEC3B. PLoS One 11, e0155391 (2016).
41. H. S. Nishimasu, et al., Engineered CRISPR-Cas9 nuclease with expanded targeting space. Science 361, 1259-1262 (2018).
42. J. H. Hu et al., Evolved Cas9 variants with broad PAM compatibility and high DNA specificity. Nature 556, 57-63 (2018).
43. T. P. Huang et al., Circularly permuted and PAM-modified Cas9 variants broaden the targeting scope of base editors. Nature Biotechnology 37, 626-631 (2019).
44. S. Q. Tsai et al., GUIDE-seq enables genome-wide profiling of off-target cleavage by CRISPR-Cas nucleases. Nat Biotechnol 33, 187-197 (2015).
45. C. Zhou et al., Off-target RNA mutation induced by DNA base editing and its elimination by mutagenesis. Nature, (2019).
46. I. Martincorena, et al., High burden and pervasive positive selection of somatic mutations in normal human skin. Science 348, 880-885 (2015).
47. J. L. Hazen et al., The Complete Genome Sequences, Unique Mutational Spectra, and Developmental Potency of Adult Neurons Revealed by Cloning. Neuron 89, 1223-1236 (2016).
48. B. Milholland et al., Differences between germline and somatic mutation rates in humans and mice. Nat Commun 8, 15183 (2017).
49. X. Dong et al., Accurate identification of single-nucleotide variants in whole-genome-amplified single cells. Nat Methods 14, 491-493 (2017).
50. M. Lynch, Evolution of the mutation rate. Trends Genet 26, 345-352 (2010).
51. R. Rahbari et al., Timing, rates and spectra of human germline mutation. Nat Genet 48, 126-133 (2016).
52. L. C. Thomason et al., Recombineering: genetic engineering in bacteria using homologous recombination. Curr Protoc Mol Biol 106, 1 16 11-39 (2014).
53. G. E. Crooks et al., WebLogo: A sequence logo generator. Genome Res. 14, 1188-1190 (2004).
54. K. Clement et al., CRISPResso2: Accurate and Rapid Analysis of Genome Editing Data from Nucleases and Base Editors. Nature Biotechnology 37, 224-226 (2019).
55. H. Li, R. Durbin, Fast and accurate short read alignment with Burrows-Wheeler transform.

Bioinformatics 25, 1754-1760 (2009).
56. B. S. Pedersen, A. R. Quinlan, Mosdepth: quick coverage calculation for genomes and exomes. Bioinformatics 34, 867-868 (2018).
57. G. A. Van der Auwera et al., From FastQ data to high confidence variant calls: the Genome Analysis Toolkit best practices pipeline. Curr Protoc Bioinformatics 43, 11-33 (2013).
58. E. Garrison, G. Marth, Haplotype-based variant detection from short-read sequencing. arXiv preprint (2012).
59. D. C. Koboldt et al., VarScan 2: somatic mutation and copy number alteration discovery in cancer by exome sequencing. Genome Res 22, 568-576 (2012).
60. I. Farrance and R. Frenkel, Uncertainty of measurement: A review of the rules for calculating uncertainty components through functional relationships. Clin. Biochem. Rev. 33, 49-75 (2012).

### OTHER EMBODIMENTS

The foregoing has been a description of certain non-limiting embodiments of the disclosure. Those of ordinary skill in the art will appreciate that various changes and modifications to this description may be made without departing from the spirit or scope of the present disclosure, as defined in the following claims.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the present disclosure, the disclosure shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of determining off-target editing frequency of a base editor comprising:
(a) contacting a nucleic acid molecule comprising a target sequence, with a first complex, wherein the first complex comprises (i) a cytosine base editor comprising a Cas9 domain, and (ii) a first guide RNA that is engineered to bind to the Cas9 domain of the cytosine base editor, wherein the first guide RNA comprises a first sequence of at least 10 contiguous nucleotides that is complementary to the target sequence;
(b) contacting the nucleic acid molecule with a second complex, wherein the second complex comprises (iii) a first nuclease inactive Cas9 (dCas9) protein, and (iv) a second guide RNA that is engineered to bind to the first dCas9 protein, wherein the second guide RNA comprises a second sequence of at least 10 contiguous nucleotides that is complementary to a third sequence, whereby the first complex and second complex generate two or more R-loops in the nucleic acid molecule, and
(c) sequencing at least a portion of the target sequence and/or at least a portion of the nucleic acid molecule comprising the third sequence.

2. The method of claim 1 further comprising contacting the nucleic acid molecule with a third, fourth, fifth, and/or sixth complex, wherein each of the third, fourth, fifth, and/or sixth complexes comprises (v) a second dCas9 protein, and (vi) a third guide RNA that is engineered to bind to the second dCas9 protein, wherein the third guide RNA comprises a fourth sequence of at least 10 contiguous nucleotides that is complementary to the third sequence.

3. The method of claim 2, wherein the second guide RNA and the third guide RNA are the same.

4. The method of any one of claims 1-3, wherein the third sequence has about 60% or less sequence identity to the target sequence.

5. The method of any one of claims 1-4, wherein the target sequence and the third sequence are within about 1000 nucleotides, 500 nucleotides, about 400 nucleotides, about 300 nucleotides, about 200 nucleotides, about 150 nucleotides, about 120 nucleotides, about 100 nucleotides, about 90 nucleotides, or about 75 nucleotides of one another.

6. The method of any one of claims 1-5, wherein the Cas9 domain is a Cas9 nickase.

7. The method of any one of claims 1-6, wherein the Cas9 domain is derived from a first bacterial species, and the first dCas9 protein and the second dCas9 protein are derived from a second bacterial species.

8. The method of any one of claims 1-7, wherein the first guide RNA comprises a sequence of at least 15 or at least 20 contiguous nucleotides that is complementary to the target sequence.

9. The method of any one of claims 1-8, wherein the second guide RNA and/or third guide RNA comprises a sequence of at least 15 or at least 20 contiguous nucleotides that is complementary to the third sequence.

10. The method of any one of claims 1-9, wherein the target sequence and the third sequence are comprised within the genome of a cell.

11. The method of any one of claims 1-10, wherein the target sequence and the third sequence are comprised within the genome of a mammalian cell.

12. The method of claim 10 or 11, wherein the step of contacting comprises transfecting the cell with one or more plasmids encoding the cytosine base editor, the first guide RNA, the first dCas9 protein, and the second guide RNA.

13. The method of claim 12, wherein the step of contacting comprises further transfecting the cell with one or more plasmids encoding the second dCas9 protein and the third guide RNA.

14. The method of claim 12 or 13, wherein the step of transfecting is performed using lipofection, nucleofection, or electroporation.

15. The method of any one of claims 7-14, wherein the first bacterial species is S. pyogenes.

16. The method of any one of claims 7-15, wherein the second bacterial species is S. aureus.

17. The method of any one of claims 10-16, wherein the cell is a population of cells.

18. The method of claim 17, wherein the step of sequencing comprises performing high-throughput sequencing of one or more portions of the genomes of the cells of the population.

19. The method of any one of claims 1-18, wherein the target sequence comprises a C:G nucleobase pair.

20. A system for determining off-target editing frequency of a base editor comprising:
one or more eukaryotic cells each comprising i) a first nucleic acid molecule encoding a cytosine base editor comprising a Cas9 domain; (ii) a second nucleic acid molecule encoding a first guide RNA that is engineered to bind to the Cas9 domain of the cytosine base editor, wherein the first guide RNA comprises a first sequence of at least 10 contiguous nucleotides that is complementary to a target sequence; (iii) a third nucleic acid molecule encoding a nuclease inactive Cas9 (dCas9) protein; and (iv) a fourth nucleic acid molecule encoding a second gRNA that is engineered to bind to the dCas9 protein, wherein the second guide RNA comprises a second sequence of at least 10 contiguous nucleotides that is complementary to a third sequence, whereby the first complex and second complex generate two or more R-loops in the nucleic acid molecule, and
wherein the third sequence has about 60% or less sequence identity to the target sequence.

21. The system of claim 20, wherein the target sequence comprises a C:G nucleobase pair.

22. The system of claim 20 or 21, wherein the Cas9 domain is a Cas9 nickase.

23. The system of any one of claims 20-22, wherein the Cas9 domain is derived from a first bacterial species, and the dCas9 protein is derived from a second bacterial species.

24. The system of claim 23, wherein the first bacterial species is S. pyogenes, and the second bacterial species is S. aureus.

25. The system of any one of claims 20-24, wherein the eukaryotic cells comprise mammalian cells.

26. A base editor comprising a) a cytidine deaminase domain, b) a napDNAbp domain, c) one or more nuclear localization signals, and d) two or more uracil glycosylase inhibitor (UGI) domains, wherein the cytidine deaminase domain is selected from YE1, YE2, YEE, EE, R33A, R33A+K34A, AALN, A3A, eA3A, A3G, and variants thereof.

27. The base editor of claim 26, wherein the napDNAbp domain is selected from a Cas9, a Cpf1, a CasX, a CasY, a C2c1, a C2c2, a C2c3, a GeoCas9, a CjCas9, a Cas12a, a Cas12b, a Cas12g, a Cas12h, a Cas12i, a Cas13b, a Cas13c, a Cas13d, a Cas14, a Csn2, an xCas9, an SpCas9-NG, a circularly permuted Cas9, an Argonaute (Ago) domain, Cas9-KKH, SmacCas9, Spy-macCas9, an SpCas9-NRRH, an SpCas9-NRCH, an SpCas9-NRTH, and variants thereof.

28. The base editor of claim 26 or 27, wherein the napDNAbp domain has nuclease activity, has nickase activity, or has no nuclease or nickase activity.

29. The base editor of claim 28, wherein the napDNAbp domain is a dCas9.

30. The base editor of claim 29, wherein the dCas9 comprises the amino acid sequence set forth in SEQ ID NO: 214.

31. The base editor of claim 28, wherein the napDNAbp domain is a Cas9 nickase (nCas9).

32. The base editor of claim 30, wherein the Cas9 nickase comprises the amino acid sequence set forth in SEQ ID NO: 215.

33. The base editor of claim 26 or 27, wherein the napDNAbp domain is a circularly permuted Cas9.

34. The base editor of claim 33, wherein the circularly permuted Cas9 is CP1028.

35. The base editor of claim 26 or 27, wherein the napDNAbp domain is an SpCas9-NG.

36. The base editor of any one of claims 26-35, wherein the napDNAbp domain is selected from any one of the amino acid sequences set forth in SEQ ID NOs: 213-229 or 235-237.

37. The base editor of any one of claims 26-36, wherein the base editor comprises two UGI domains.

38. The base editor of any one of claims 26-37, wherein the two or more nuclear localization sequences (NLS) comprises the amino acid sequence KRTADGSEFESPKKKRKV (SEQ ID NO: 285) or KRTADGSEFEPKKKRKV (SEQ ID NO: 286).

39. The base editor of any one of claims 26-38, wherein the base editor comprises the structure: NH₂-[first nuclear localization sequence]-[cytidine deaminase domain]-[napDNAbp domain]-[first UGI domain]-[second UGI domain]-[second nuclear localization sequence]-COOH, wherein each instance of “]-[” indicates the presence of an optional linker sequence.

40. The base editor of claim 39, wherein the cytidine deaminase domain and the napDNAbp domain are linked via a linker comprising the amino acid sequence SGGSSGGSSGSETPGTSESATPESSGGSSGGS (SEQ ID NO: 301); the napDNAbp domain and the first UGI domain are linked via a linker comprising the amino acid sequence of SGGSGGSGGS (SEQ ID NO: 302); the first UGI domain and the second UGI domain are linked via a linker comprising the amino acid sequence of SGGSGGSGGS (SEQ ID NO: 302); and/or the second UGI domain and the second nuclear localization sequence are linked via a linker comprising the amino acid sequence of SGGS (SEQ ID NO: 303).

41. The base editor of any one of claims 26-40, wherein the cytidine deaminase domain comprises YE1.

42. The base editor of any one of claims 26-40, wherein the cytidine deaminase domain is selected from YE1, YE2, YEE, EE, R33A, or R33A+K34A and variants thereof, and the napDNAbp domain is selected from an nCas9, an xCas9, an SpCas9-NG, or a CP1028.

43. The base editor of any one of claims 26-42, wherein the cytidine deaminase domain is YE1 and the napDNAbp domain is selected from an nCas9 or a CP1028.

44. The base editor of any one of claims 26-43, wherein the base editor comprises an amino acid sequence that has at least 85% sequence identity, at least 90% sequence identity, at least 95% sequence identity, at least 97.5% sequence identity, or at least 99.5% sequence identity to any one of SEQ ID NOs: 257-282.

45. The base editor of any one of claims 26-44, wherein the base editor comprises any one of the amino acid sequences set forth in SEQ ID NOs: 257-282.

46. The base editor of any one of claims 26-45, wherein the base editor is codon-optimized for expression in human cells.

47. The base editor any one of claims 26-46, wherein the base editor provides an off-target editing frequency of less than 1.5% after being contacted with a nucleic acid molecule comprising a target sequence.

48. A method for editing a target nucleobase pair comprising:
contacting a target sequence in a nucleic acid molecule with the base editor of any one of claims 26-47, and a guide RNA (gRNA);
deaminating a cytosine in a target nucleobase pair within the target sequence, and
obtaining a frequency of off-target editing of less than 1.5%.

49. The method of claim 48, wherein the off-target editing frequency is less than 1.25%, less than 1.1%, less than 1%, less than 0.75%, less than 0.5%, less than 0.4%, less than 0.25%, less than 0.2%, less than 0.15%, less than 0.1%, less than 0.05%, or less than 0.025%.

50. The method of claim 48 or 49, wherein the off-target editing frequency is about 0.4% or less.

51. The method of any one of claims 48-50, further comprising obtaining a frequency of editing of less than 1.5% in sequences having 60% or less sequence identity to the target sequence.

52. The method of any one of claims 48-51 further comprising obtaining an on-target editing frequency of greater than 50% at the target nucleobase pair.

53. The method of any one of claims 48-52 further comprising obtaining an on-target editing frequency of greater than 65% at the target nucleobase pair.

54. The method of any one of claims 48-53 further comprising obtaining an on-target editing frequency of greater than 85% at the target nucleobase pair.

55. The method of any one of claims 48-54, wherein the step of contacting results in an indel frequency of less than 0.75%, less than 0.6%, less than 0.5%, less than 0.4%, less than 0.3%, or less than 0.2% in the nucleic acid molecule.

56. The method of any one of claims 48-55, wherein the step of contacting results in an indel frequency of 0.5% or less in the nucleic acid molecule.

57. The method of any one of claims 48-56, wherein the gRNA comprises a sequence of at least 10, at least 15, or at least 20 contiguous nucleotides that is complementary to the target sequence.

58. The method of any one of claims 48-57, wherein the step of contacting is performed in vitro.

59. The method of any one of claims 48-58, wherein the step of contacting is performed in vivo in a subject.

60. The method of claim 59, wherein the subject is a human.

61. The method of any one of claims 48-60, wherein the target sequence is in the genome of an organism.

62. The method of claim 61, wherein the organism is bacteria.

63. The method of claim 61, wherein the organism is a eukaryote.

64. The method of claim 63, wherein the organism is a plant or a fungus.

65. The method of claim 63, wherein the organism is a vertebrate.

66. The method of claim 65, wherein the vertebrate is a mammal.

67. The method of claim 66, wherein the mammal is a mouse, a rat, or a human.

68. The method of claim 67, wherein the mammal is a human.

69. The method of any one of claims 48-68, wherein the nucleic acid molecule is double-stranded DNA.

70. The method of any one of claims 48-69, wherein the target sequence comprises a sequence associated with a disease or disorder.

71. The method of any one of claims 48-70, wherein the target sequence comprises a point mutation associated with a disease or disorder.

72. The method of claim 71, wherein the point mutation is a T-to-C or an A-to-G mutation.

73. The method of claim 71 or 72, wherein the step of contacting results in a correction of the point mutation.

74. The method of claim 59, wherein the subject has a T-to-C, or an A-to-G mutation, that is associated with a disease, disorder, or condition.

75. The method of claim 74, wherein the C of the T-to-C mutation is converted to a T.

76. The method of claim 74, wherein the G of the A-to-G mutation is converted to an A.

77. The method of any one of claims 48-76, wherein the base editor and gRNA are administered as a protein:RNA complex.

78. A polynucleotide encoding the base editor of any one of claims 26-47.

79. A vector comprising the polynucleotide of claim 78.

80. The vector of claim 79, wherein the vector comprises a heterologous promoter driving expression of the polynucleotide.

81. A complex comprising the base editor of any one of claims 26-47 in association with a guide RNA.

82. The complex of claim 81, wherein the guide RNA is 15-100 nucleotides long and comprises a sequence of at least 10, at least 15, or at least 20 contiguous nucleotides that is complementary to a target sequence.

83. A cell comprising the base editor of any one of claims 26-47.

84. A cell comprising the polynucleotide of claim 78.

85. A cell comprising the vector of claim 79 or 80.

86. A cell comprising the complex of claim 81 or 82.

87. A pharmaceutical composition comprising the base editor of any one of claims 26-47 and a pharmaceutically acceptable excipient.

88. A pharmaceutical composition comprising the polynucleotide of claim 78 and a pharmaceutically acceptable excipient.

89. A pharmaceutical composition comprising the vector of claim 79 or 80, and a pharmaceutically acceptable excipient.

90. A pharmaceutical composition comprising the complex of claim 81 or 82, and a pharmaceutically acceptable excipient.

91. A kit comprising a nucleic acid construct comprising:
(i) a nucleic acid sequence encoding the base editor of any one of claims 26-47;
(ii) a nucleic acid sequence encoding a gRNA; and
(iii) one or more heterologous promoters that drive the expression of the sequence of (i) and/or the sequence of (ii).

92. The kit of claim 91 further comprising an expression construct encoding a guide RNA backbone, wherein the construct comprises a cloning site positioned to allow the cloning of a nucleic acid sequence identical or complementary to a target sequence into the guide RNA backbone.

93. A kit comprising a nucleic acid construct comprising:
(i) a nucleic acid sequence encoding a cytosine base editor comprising a Cas9 domain;
(ii) a nucleic acid sequence encoding a first gRNA that is engineered to bind to the Cas9 domain of the cytosine base editor, wherein the second guide RNA comprises a first sequence of at least 10 contiguous nucleotides that is complementary to a target sequence;
(iii) a nucleic acid sequence encoding a first nuclease inactive Cas9 (dCas9) protein; and
(iv) a nucleic acid sequence encoding a second gRNA that is engineered to bind to the dCas9 protein, wherein the second guide RNA comprises a second sequence of at least 10 contiguous nucleotides that is complementary to a third sequence, wherein the third sequence has about 60% or less sequence identity to the target sequence.

94. The kit of claim 93 further comprising a nucleic acid construct comprising:
(v) a nucleic acid sequence encoding a second dCas9 protein; and
(vi) a nucleic acid sequence encoding a third gRNA that is engineered to bind to a second dCas9 protein, wherein the third guide RNA comprises a fourth sequence of at least 10 contiguous nucleotides that is complementary to the third sequence.

95. A system for determining off-target editing frequency of a base editor, comprising one or more prokaryotic cells comprising:
(i) a nucleic acid molecule that contains a target sequence within a first inactive antibiotic resistance gene, wherein the target sequence within the first inactive antibiotic resistance gene contains a first mutant nucleotide base that yields an active antibiotic resistance gene conferring resistance to a first antibiotic when the first mutant nucleotide base is mutated to a different nucleotide base;
(ii) a second nucleic acid molecule that contains a non-target sequence within a second inactive antibiotic resistance gene, wherein the non-target sequence within the second inactive antibiotic resistance gene contains a second mutant nucleotide base that yields an active antibiotic resistance gene conferring resistance to a second antibiotic when the second mutant nucleotide base is mutated to a different base; and
(iii) a third nucleic acid molecule encoding a cytosine base editor and a guide RNA comprising a sequence of at least 10 contiguous nucleotides that is complementary to the target sequence within the first inactive antibiotic resistance gene.

96. The system of claim 95, wherein the first nucleic acid molecule of (i) is comprised within a plasmid.

97. The system of claim 95 or 96, wherein the second nucleic acid molecule of (ii) is comprised in the genome of the one or more prokaryotic cells.

98. The system of any one of claims 95-97, wherein the first mutant nucleotide is a cytosine and wherein mutating the cytosine to a thymine yields an active antibiotic resistance gene conferring resistance to the first antibiotic.

99. The system of any one of claims 95-98, wherein the second mutant nucleotide is a cytosine and wherein mutating the cytosine to a thymine yields an active antibiotic resistance gene conferring resistance to the second antibiotic.

100. The system of any one of claims 95-99, wherein the first antibiotic is chloramphenicol.

101. The system of any one of claims 95-100, wherein the second antibiotic is rifampin.

102. The system of any one of claims 95-101, wherein the cytosine base editor comprises a nuclease inactive Cas9 (dCas9) domain.

103. A method of determining off-target editing frequency of a base editor in accordance with the system of any one of claims 95-102 comprising:
contacting a prokaryotic cell that comprises the second nucleic acid molecule, with
(i) the first nucleic acid molecule and
(ii) the third nucleic acid molecule; and
further contacting the prokaryotic cell with a growth medium comprising the second antibiotic and/or the first antibiotic.

104. The method of claim 103, wherein the second nucleic acid molecule is in the genome of the prokaryotic cell.

105. Use of (a) the base editor of any one of claims 26-47, and (b) a guide RNA targeting the base editor of (a) to a target C:G nucleobase pair in DNA editing.

106. The base editor of any one of claims 26-47, a complex of claim 81 or 82, or a pharmaceutical composition of any one of claims 87-90, for use as a medicament.

107. A method of determining off-target editing frequency of a base editor comprising:
(a) contacting a nucleic acid molecule comprising a target sequence, with a first complex, wherein the first complex comprises (i) an adenine base editor comprising a Cas9 domain, and (ii) a first guide RNA that is engineered to bind to the Cas9 domain of the adenine base editor, wherein the first guide RNA comprises a first sequence of at least 10 contiguous nucleotides that is complementary to the target sequence;
(b) contacting the nucleic acid molecule with a second complex, wherein the second complex comprises (iii) a first nuclease inactive Cas9 (dCas9) protein, and (iv) a second guide RNA that is engineered to bind to the first dCas9 protein, wherein the second guide RNA comprises a second sequence of at least 10 contiguous nucleotides that is complementary to a third sequence, whereby the first complex and second complex generate two or more R-loops in the nucleic acid molecule, and
(c) sequencing at least a portion of the target sequence and/or at least a portion of the nucleic acid molecule comprising the third sequence.

108. A system for determining off-target editing frequency of a base editor comprising:
one or more eukaryotic cells each comprising i) a first nucleic acid molecule encoding an adenine base editor comprising a Cas9 domain; (ii) a second nucleic acid molecule encoding a first guide RNA that is engineered to bind to the Cas9 domain of the adenine base editor, wherein the first guide RNA comprises a first sequence of at least 10 contiguous nucleotides that is complementary to a target sequence; (iii) a third nucleic acid molecule encoding a nuclease inactive Cas9 (dCas9) protein; and (iv) a fourth nucleic acid molecule encoding a second gRNA that is engineered to bind to the dCas9 protein, wherein the second guide RNA comprises a second sequence of at least 10 contiguous nucleotides that is complementary to a third sequence, whereby the first complex and second complex generate two or more R-loops in the nucleic acid molecule, and
wherein the third sequence has about 60% or less sequence identity to the target sequence.
